(19) United States
(12) Patent Application Publication
(10) Pub. No.: US 2026/0265228 A1
(43) Pub. Date: Sep. 10, 2026

(54) HETEROCYCLIC-CARBONYL-CYCLIC COMPOUNDS AS MAGL INHIBITORS

(71) Applicant: Hoffmann-La Roche Inc., Little Falls, NJ (US)

(72) Inventors: Machoud AMOUSSA, Berlin (DE); Joerg BENZ, Rheinfelden (DE); Jason Jacques DENIZOT, Savigny-en-Sancerre (FR); Kallie FRISTON, Stockton-On-Tees (GB); Rudolf Liun Zaccaria GANZONI, Zürich (CH); Maude GIROUD, Basel (CH); Uwe Michael GRETHER, Efringen-Kirchen (DE); Benoit HORNSPERGER, Altkirch (FR); Isabelle KAUFMANN, Wettingen (CH); Carsten KROLL, Basel (CH); Bernd KUHN, Reinach BL (CH); Martin KURATLI, Birsfelden (CH); Rainer Eugen MARTIN, Basel (CH); Fionn Susannah O’HARA, Basel (CH); Bernd PUELLMANN, Aesch (CH); Martin RITTER, Mumpf (CH); Didier ROMBACH, Mulhouse (FR); Philipp Claudio SCHMID, Birsfelden (CH); Hannan SEYAL, London (GB); Matthias Beat WITTWER, Riehen (CH)

(73) Assignee: Hoffmann-La Roche Inc., Little Falls, NJ (US)

(21) Appl. No.: 18/877,440

(22) PCT Filed: Jun. 22, 2023
(86) PCT No.: PCT/EP2023/066906

§ 371 Date: Dec. 20, 2024

(30) Foreign Application Priority Data
Jun. 24, 2022 (EP) 22181024.5

(51) Int. Cl. C07D 403/14; A61K 31/4025; A61K 31/41; A61K 31/4155; A61K 31/4178; A61K 31/4192; A61K 31/4196; A61K 31/422; A61K 31/4245; A61K 31/427; A61K 31/433; A61K 31/437; A61K 31/438; A61K 31/4439; A61K 31/454; A61K 31/496; A61K 31/497; A61K 31/501; A61K 31/506; A61K 31/5377; A61K 31/55; C07D 401/14; C07D 403/04; C07D 403/06; C07D 403/10; C07D 405/14; C07D 413/06; C07D 413/14; C07D 417/14; C07D 471/04; C07D 471/10; C07D 487/10; C07D 491/107

(52) U.S. Cl. CPC C07D 403/14; A61K 31/4025; A61K 31/41; A61K 31/4155; A61K 31/4178; A61K 31/4192; A61K 31/4196; A61K 31/422; A61K 31/4245; A61K 31/427; A61K 31/433; A61K 31/437; A61K 31/438; A61K 31/4439; A61K 31/454; A61K 31/496; A61K 31/497; A61K 31/501; A61K 31/506; A61K 31/5377; A61K 31/55; C07D 401/14; C07D 403/04; C07D 403/06; C07D 403/10; C07D 405/14; C07D 413/06; C07D 413/14; C07D 417/14; C07D 471/04; C07D 471/10; C07D 487/10; C07D 491/107

(57) ABSTRACT

The present invention provides compounds of formula (I) CB(I) or pharmaceutically acceptable salts thereof, wherein R³ to R⁵, R^{4a}, B and C are as described herein, compositions including the compounds, processes of manufacturing the compounds and methods of using the compounds.

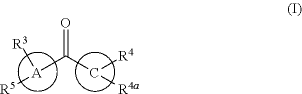

### FIELD OF THE INVENTION

The present invention relates to organic compounds useful for therapy or prophylaxis in a mammal, and in particular to monoacylglycerol lipase (MAGL) inhibitors for the treatment or prophylaxis of neuroinflammation, neurodegenerative diseases, pain, cancer, mental disorders, multiple sclerosis, Alzheimer's disease, Parkinson's disease, amyotrophic lateral sclerosis, traumatic brain injury, neurotoxicity, stroke, epilepsy, anxiety, migraine, depression, inflammatory bowel disease, abdominal pain, abdominal pain associated with irritable bowel syndrome and/or visceral pain in a mammal.

### BACKGROUND OF THE INVENTION

Endocannabinoids (ECs) are signaling lipids that exert their biological actions by interacting with cannabinoid receptors (CBRs), CB1 and CB2. They modulate multiple physiological processes including neuroinflammation, neurodegeneration and tissue regeneration (Iannotti, F. A., et al., Progress in lipid research 2016, 62, 107-28). In the brain, the main endocannabinoid, 2-arachidonoylglycerol (2-AG), is produced by diacylglycerol lipases (DAGL) and hydrolyzed by the monoacylglycerol lipase, MAGL. MAGL hydrolyses 85% of 2-AG; the remaining 15% being hydrolysed by ABHD6 and ABDH12 (Nomura, D. K., et al., Science 2011, 334, 809). MAGL is expressed throughout the brain and in most brain cell types, including neurons, astrocytes, oligodendrocytes and microglia cells (Chanda, P. K., et al., Molecular pharmacology 2010, 78, 996; Viader, A., et al., Cell reports 2015, 12, 798). 2-AG hydrolysis results in the formation of arachidonic acid (AA), the precursor of prostaglandins (PGs) and leukotrienes (LTs). Oxidative metabolism of AA is increased in inflamed tissues. There are two principal enzyme pathways of arachidonic acid oxygenation involved in inflammatory processes, the cyclo-oxygenase which produces PGs and the 5-lipoxygenase which produces LTs. Of the various cyclooxygenase products formed during inflammation, PGE2 is one of the most important. These products have been detected at sites of inflammation, e.g. in the cerebrospinal fluid of patients suffering from neurodegenerative disorders and are believed to contribute to inflammatory response and disease progression. Mice lacking MAGL (Mgll−/−) exhibit dramatically reduced 2-AG hydrolase activity and elevated 2-AG levels in the nervous system while other arachidonoyl-containing phospho- and neutral lipid species including anandamide (AEA), as well as other free fatty acids, are unaltered. Conversely, levels of AA and AA-derived prostaglandins and other eicosanoids, including prostaglandin E2 (PGE2), D2 (PGD2), F2 (PGF2), and thromboxane B2 (TXB2), are strongly decreased. Phospholipase A₂ (PLA₂) enzymes have been viewed as the principal source of AA, but cPLA₂-deficient mice have unaltered AA levels in their brain, reinforcing the key role of MAGL in the brain for AA production and regulation of the brain inflammatory process.

Neuroinflammation is a common pathological change characteristic of diseases of the brain including, but not restricted to, neurodegenerative diseases (e.g. multiple sclerosis, Alzheimer's disease, Parkinson disease, amyotrophic lateral sclerosis, traumatic brain injury, neurotoxicity, stroke, epilepsy and mental disorders such as anxiety and migraine). In the brain, production of eicosanoids and prostaglandins controls the neuroinflammation process. The pro-inflammatory agent lipopolysaccharide (LPS) produces a robust, time-dependent increase in brain eicosanoids that is markedly blunted in Mgll−/− mice. LPS treatment also induces a widespread elevation in pro-inflammatory cytokines including interleukin-1-a (IL-1-a), IL-1b, IL-6, and tumor necrosis factor-a (TNF-a) that is prevented in Mgll−/− mice.

Neuroinflammation is characterized by the activation of the innate immune cells of the central nervous system, the microglia and the astrocytes. It has been reported that anti-inflammatory drugs can suppress in preclinical models the activation of glia cells and the progression of disease including Alzheimer's disease and multiple sclerosis (Lleo A., Cell Mol Life Sci. 2007, 64, 1403). Importantly, genetic and/or pharmacological disruption of MAGL activity also blocks LPS-induced activation of microglial cells in the brain (Nomura, D. K., et al., Science 2011, 334, 809).

In addition, genetic and/or pharmacological disruption of MAGL activity was shown to be protective in several animal models of neurodegeneration including, but not restricted to, Alzheimer's disease, Parkinson's disease and multiple sclerosis. For example, an irreversible MAGL inhibitor has been widely used in preclinical models of neuroinflammation and neurodegeneration (Long, J. Z., et al., Nature chemical biology 2009, 5, 37). Systemic injection of such inhibitor recapitulates the Mgll−/− mice phenotype in the brain, including an increase in 2-AG levels, a reduction in AA levels and related eicosanoids production, as well as the prevention of cytokines production and microglia activation following LPS-induced neuroinflammation (Nomura, D. K., et al., Science 2011, 334, 809), altogether confirming that MAGL is a druggable target.

Consecutive to the genetic and/or pharmacological disruption of MAGL activity, the endogenous levels of the MAGL natural substrate in the brain, 2-AG, are increased. 2-AG has been reported to show beneficial effects on pain with, for example, anti-nociceptive effects in mice (Ignatowska-Jankowska B. et al., J. Pharmacol. Exp. Ther. 2015, 353, 424) and on mental disorders, such as depression in chronic stress models (Zhong P. et al., Neuropsychopharmacology 2014, 39, 1763).

Furthermore, oligodendrocytes (OLs), the myelinating cells of the central nervous system, and their precursors (OPCs) express the cannabinoid receptor 2 (CB2) on their membrane. 2-AG is the endogenous ligand of CB1 and CB2 receptors. It has been reported that both cannabinoids and pharmacological inhibition of MAGL attenuate OLs's and OPCs's vulnerability to excitotoxic insults and therefore may be neuroprotective (Bernal-Chico, A., et al., Glia 2015, 63, 163). Additionally, pharmacological inhibition of MAGL increases the number of myelinating OLs in the brain of mice, suggesting that MAGL inhibition may promote differentiation of OPCs in myelinating OLs in vivo (Alpar, A., et al., Nature communications 2014, 5, 4421). Inhibition of MAGL was also shown to promote remyelination and functional recovery in a mouse model of progressive multiple sclerosis (Feliu A. et al., Journal of Neuroscience 2017, 37 (35), 8385).

In addition, in recent years, metabolism is talked highly important in cancer research, especially the lipid metabolism. Researchers believe that the de novo fatty acid synthesis plays an important role in tumor development. Many studies illustrated that endocannabinoids have anti-tumorigenic actions, including anti-proliferation, apoptosis induction and anti-metastatic effects. MAGL as an important decomposing enzyme for both lipid metabolism and the endocannabinoids system, additionally as a part of a gene expression signature, contributes to different aspects of tumorigenesis, including in glioblastoma (Qin, H. et al., Cell Biochem. Biophys. 2014, 70, 33; Nomura, D. K. et al., Cell 2009, 140, 49; Nomura, D. K. et al., Chem. Biol. 2011, 18, 846; Jinlong, Y. et al., Nat. Commun. 2020, 11, 2978).

The endocannabinoid system is also involved in many gastrointestinal physiological and physiopathological actions (Marquez, L. et al., PLoS One 2009, 4, e6893). All these effects are driven mainly via cannabinoid receptors (CBRs), CB1 and CB2. CB1 receptors are present throughout the GI tract of animals and healthy humans, especially in the enteric nervous system (ENS) and the epithelial lining, as well as smooth muscle cells of blood vessels in the colonic wall (Wright, K. et al., Gastroenterology 2005, 129, 437; Duncan, M. et al., Aliment. Pharmacol. Ther. 2005, 22, 667). Activation of CB1 produces anti-emetic, anti-motility, and anti-inflammatory effect, and help to modulate pain (Perisetti, A. et al., Ann. Gastroenterol. 2020, 33, 134). CB2 receptors are expressed in immune cells such as plasma cells and macrophages, in the lamina propria of the GI tract (Wright, K. et al., Gastroenterology 2005, 129, 437), and primarily on the epithelium of human colonic tissue associated with inflammatory bowel disease (IBD). Activation of CB2 exerts anti-inflammatory effect by reducing pro-inflammatory cytokines. Expression of MAGL is increased in colonic tissue in UC patients (Marquez, L. et al., PLoS One 2009, 4, e6893) and 2-AG levels are increased in plasma of IBD patients (Grill, M. et al., Sci. Rep. 2019, 9, 2358). Several animal studies have demonstrated the potential of MAGL inhibitors for symptomatic treatment of IBD. MAGL inhibition prevents TNBS-induced mouse colitis and decreases local and circulating inflammatory markers via a CB1/CB2 MoA (Marquez, L. et al., PLoS One 2009, 4, e6893). Furthermore, MAGL inhibition improves gut wall integrity and intestinal permeability via a CB1 driven MoA (Wang, J. et al., Biochem. Biophys. Res. Commun. 2020, 525, 962).

In conclusion, suppressing the action and/or the activation of MAGL is a promising new therapeutic strategy for the treatment or prevention of neuroinflammation, neurodegenerative diseases, pain, cancer, mental disorders, inflammatory bowel disease, abdominal pain and abdominal pain associated with irritable bowel syndrome. Furthermore, suppressing the action and/or the activation of MAGL is a promising new therapeutic strategy for providing neuroprotection and myelin regeneration. Accordingly, there is a high unmet medical need for new MAGL inhibitors.

### SUMMARY OF THE INVENTION

In a first aspect, the present invention provides compounds of formula (I)

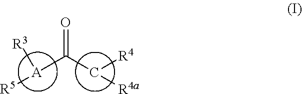

or pharmaceutically acceptable salts thereof, wherein R³ to R⁵, R^{4a}, B and C are as described herein.

In one aspect, the present invention provides a process of manufacturing a compound of formula (IA) or (IB) described herein, comprising:
(a) reacting an amine of formula 2, wherein p, q, and R⁴ are as described herein,

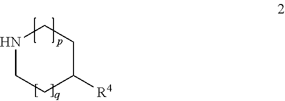

with a carboxylic acid 3a, wherein L, A, B, and R¹ to R³ are as described herein

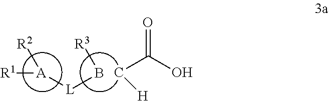

in the presence of a coupling reagent, such as CDI, DCC, HATU, HBTU, HOBT, TBTU, T₃P or Mukaiyama reagent, and optionally in the presence of a base, such as TEA, DIPEA (Huenig's base) or DMAP, to form said compound of formula (IB), wherein A, B, L, p, q, and R¹ to R⁴ are as defined herein

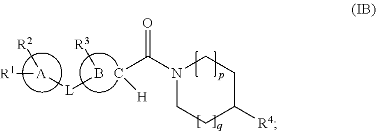

or
(b) reacting an amine of formula 2, wherein p, q, and R⁴ are as described herein,

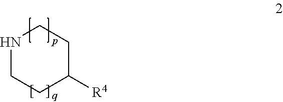

with a carboxylic acid chloride 3b, wherein L, A, B, and R¹ to R³ are as described herein

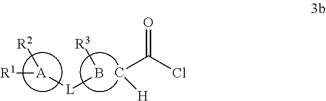

in the presence of a base, such as TEA, Huenig's base, pyridine, DMAP or lithium bis(trimethylsilyl)amide, to form said compound of formula (IB), wherein A, B, L, p, q, and R¹ to R⁴ are as defined herein; or
(c) reacting a first amine of formula 1, wherein A, B, L, and R¹ to R³ are as described herein,

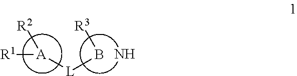

with a second amine 2, wherein X, Y, and Z are as described herein

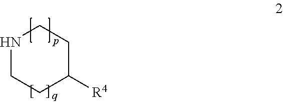

in the presence of a base, such as sodium bicarbonate, and a urea forming reagent, such as bis(trichloromethyl) carbonate, phosgene, trichloromethyl chloroformate, (4-nitrophenyl) carbonate or 1,1′-carbonyldiimidazole, to form said compound of formula (IA), wherein A, B, L, p, q, and R¹ to R⁴ are as defined herein

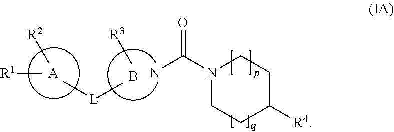

In a further aspect, the present invention provides a compound of formula (I) as described herein, when manufactured according to the processes described herein.

In a further aspect, the present invention provides a compound of formula (I) as described herein, for use as therapeutically active substance.

In a further aspect, the present invention provides a pharmaceutical composition comprising a compound of formula (I) as described herein and a therapeutically inert carrier.

In a further aspect, the present invention provides the use of a compound of formula (I) as described herein or of a pharmaceutical composition described herein for inhibiting monoacylglycerol lipase (MAGL) in a mammal.

In a further aspect, the present invention provides the use of a compound of formula (I) as described herein, or a pharmaceutically acceptable salt thereof, or of a pharmaceutical composition described herein for the treatment or prophylaxis of neuroinflammation, neurodegenerative diseases, pain, cancer, mental disorders and/or inflammatory bowel disease in a mammal.

In a further aspect, the present invention provides the use of a compound of formula (I) as described herein, or a pharmaceutically acceptable salt thereof, or of a pharmaceutical composition described herein for the treatment or prophylaxis of multiple sclerosis, Alzheimer's disease, Parkinson's disease, amyotrophic lateral sclerosis, traumatic brain injury, neurotoxicity, stroke, epilepsy, anxiety, migraine, depression, hepatocellular carcinoma, colon carcinogenesis, ovarian cancer, neuropathic pain, chemotherapy induced neuropathy, acute pain, chronic pain, spasticity associated with pain, abdominal pain, abdominal pain associated with irritable bowel syndrome and/or visceral pain in a mammal.

### DETAILED DESCRIPTION OF THE INVENTION

### Definitions

Features, integers, characteristics, compounds, chemical moieties or groups described in conjunction with a particular aspect, embodiment or example of the invention are to be understood to be applicable to any other aspect, embodiment or example described herein, unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

The term “alkyl” refers to a mono- or multivalent, e.g., a mono- or bivalent, linear or branched saturated hydrocarbon group of 1 to 12 carbon atoms. In some preferred embodiments, the alkyl group contains 1 to 6 carbon atoms (“C₁₋₆-alkyl”), e.g., 1, 2, 3, 4, 5, or 6 carbon atoms. In other embodiments, the alkyl group contains 1 to 3 carbon atoms, e.g., 1, 2 or 3 carbon atoms. Some non-limiting examples of alkyl include methyl, ethyl, propyl, 2-propyl (isopropyl), n-butyl, isobutyl, sec-butyl, tert-butyl, and 2,2-dimethylpropyl. A particularly preferred, yet non-limiting example of alkyl is methyl.

The term “alkoxy” refers to an alkyl group, as previously defined, attached to the parent molecular moiety via an oxygen atom. Unless otherwise specified, the alkoxy group contains 1 to 12 carbon atoms. In some preferred embodiments, the alkoxy group contains 1 to 6 carbon atoms (“C₁₋₆-alkoxy”). In other embodiments, the alkoxy group contains 1 to 4 carbon atoms. In still other embodiments, the alkoxy group contains 1 to 3 carbon atoms. Some non-limiting examples of alkoxy groups include methoxy, ethoxy, n-propoxy, isopropoxy, n-butoxy, isobutoxy and tert-butoxy. A particularly preferred, yet non-limiting example of alkoxy is methoxy.

The term “halogen” or “halo” refers to fluoro (F), chloro (Cl), bromo (Br), or iodo (I). Preferably, the term “halogen” or “halo” refers to fluoro (F), chloro (Cl) or bromo (Br). Particularly preferred, yet non-limiting examples of “halogen” or “halo” are fluoro (F) and chloro (Cl).

The term “cycloalkyl” as used herein refers to a saturated monocyclic or bicyclic hydrocarbon group of 3 to 10 ring carbon atoms (“C₃₋₁₀-cycloalkyl”). In some preferred embodiments, the cycloalkyl group is a monocyclic hydrocarbon group of 3 to 8 ring carbon atoms. “Bicyclic cycloalkyl” refers to cycloalkyl moieties consisting of two saturated carbocycles having two carbon atoms in common, i.e., the bridge separating the two rings is either a single bond or a chain of one or two ring atoms, and to spirocyclic moieties, i.e., the two rings are connected via one common ring atom. Preferably, the cycloalkyl group is a saturated monocyclic hydrocarbon group of 3 to 6 ring carbon atoms, e.g., of 3, 4, 5 or 6 carbon atoms. Some non-limiting examples of cycloalkyl include spiro[3.3]heptan-2-yl, cyclopropyl, cyclobutyl, cyclopentyl, cyclohexyl and cycloheptyl. Preferred, yet non-limiting examples of cycloalkyl include spiro[3.3]heptan-2-yl and cyclopropyl.

The terms “heterocyclyl” and “heterocycloalkyl” are used herein interchangeably and refer to a saturated mono- or bicyclic, preferably monocyclic ring system of 3 to 10 ring atoms, preferably 3 to 8 ring atoms, wherein 1, 2, or 3 of said ring atoms are heteroatoms selected from N, O and S, the remaining ring atoms being carbon. Preferably, 1 to 2 of said ring atoms are selected from N and O, the remaining ring atoms being carbon. “Bicyclic heterocyclyl” refers to heterocyclic moieties consisting of two cycles having two ring atoms in common, i.e., the bridge separating the two rings is either a single bond or a chain of one or two ring atoms, and to spirocyclic moieties, i.e., the two rings are connected via one common ring atom. Some non-limiting examples of heterocyclyl groups include azetidin-3-yl, azetidin-2-yl, oxetan-3-yl, oxetan-2-yl, 1-piperidyl, 2-piperidyl, 3-piperidyl, 4-piperidyl, piperazinyl, pyrrolidinyl, oxazolidinyl, dihydropyrazinyl (e.g., 1,2-dihydropyrazin-6-yl), morpholinyl, 2-azaspiro[3.3]heptan-2-yl, 7-azaspiro[3.5]nonan-7-yl, 8-azabicyclo[3.2.1]octan-8-yl, 8-oxa-3-azabicyclo[3.2.1]octan, and 3,3a,4,5,6,6a-hexahydro-1H-cyclopenta[c]pyrrol-2-yl. Preferred, yet non-limiting examples of heterocyclyl groups include azetidinyl, piperazinyl, pyrrolidinyl, oxazolidinyl, dihydropyrazinyl, 2-azaspiro[3.3]heptan-2-yl, 7-azaspiro[3.5]nonan-7-yl, 8-azabicyclo[3.2.1]octan-8-yl, and 3,3a,4,5,6,6a-hexahydro-1H-cyclopenta[c]pyrrol-2-yl.

The term “aryl” refers to a monocyclic, bicyclic, or tricyclic carbocyclic ring system having a total of 6 to 14 ring members (“C₆₋₁₄-aryl”), preferably, 6 to 12 ring members, and more preferably 6 to 10 ring members, and wherein at least one ring in the system is aromatic. Some non-limiting examples of aryl include phenyl and 9H-fluorenyl (e.g. 9H-fluoren-9-yl). A particularly preferred, yet non-limiting example of aryl is phenyl.

The term “heteroaryl” refers to a mono- or multivalent, monocyclic or bicyclic ring system having a total of 5 to 14 ring members, preferably, 5 to 12 ring members, and more preferably 5 to 10 ring members, wherein at least one ring in the system is aromatic, and at least one ring in the system contains one or more heteroatoms. Preferably, “heteroaryl” refers to a 5-10 membered heteroaryl comprising 1, 2, 3 or 4 heteroatoms independently selected from O, S and N. More preferably, “heteroaryl” refers to a 5-10 membered heteroaryl, e.g. a 5-, 6-, 7-, 8-, 9- or 10-membered heteroaryl, comprising 1 to 2 heteroatoms independently selected from O, S and N. Most preferably, “heteroaryl” refers to a 5-6 membered heteroaryl comprising 1 to 2 heteroatoms independently selected from O, S and N. Some preferred, yet non-limiting examples of heteroaryl include thiazolyl (e.g. thiazol-2-yl); oxazolyl (e.g. oxazol-2-yl); oxadiazolyl; 5,6-dihydro-4H-cyclopenta[d]thiazol-2-yl; 1,2,4-oxadiazol-5-yl; pyridyl (e.g. 2-pyridyl); pyrazolyl (e.g. pyrazol-1-yl); triazolyl; tetrazolyl; pyrazinyl; imidazolyl (e.g. imidazole-1-yl); benzoxazolyl (e.g. benzoxazol-2-yl), 2,3-dihydrobenzofuranyl; and oxazolo[5,4-c]pyridin-2-yl. Some particularly preferred, yet non-limiting examples of heteroaryl include oxadiazolyl; pyridyl; triazolyl; tetrazolyl; pyrazinyl and imidazolyl.

A heterocyclic or heteroaromatic ring can be attached to its pendant group at any heteroatom or carbon atom that results in a stable structure.

The term “hydroxy” refers to an —OH group.

The term “amino” refers to an —NH₂ group.

The term “cyano” refers to a —CN (nitrile) group.

The term “carbamoyl” refers to a group —C(O)—NH₂.

The term “oxo” refers to a group ═O.

The term “carbonyl” refers to a group C═O.

The term “haloalkyl” refers to an alkyl group, wherein at least one of the hydrogen atoms of the alkyl group has been replaced by a halogen atom, preferably fluoro. Preferably, “haloalkyl” refers to an alkyl group wherein 1, 2 or 3 hydrogen atoms of the alkyl group have been replaced by a halogen atom, most preferably fluoro. Preferred, yet non-limiting examples of haloalkyl are fluoromethyl, difluoromethyl, trifluoromethyl, and trifluoroethyl (e.g. 2,2,2-trifluoroethyl). A preferred, yet non-limiting example of haloalkyl is trifluoromethyl.

The term “haloalkoxy” refers to an alkoxy group, wherein at least one of the hydrogen atoms of the alkoxy group has been replaced by a halogen atom, preferably fluoro. Preferably, “haloalkoxy” refers to an alkoxy group wherein 1, 2 or 3 hydrogen atoms of the alkoxy group have been replaced by a halogen atom, most preferably fluoro. Preferred, yet non limiting examples of haloalkoxy are 2,2,2-trifluoro-1,1-dimethyl-ethoxy, 2,2,2-trifluoroethoxy, and trifluoromethoxy.

The term “haloaryl” refers to an aryl group, wherein at least one of the hydrogen atoms of the aryl group has been replaced by a halogen atom, preferably fluoro or chloro. Preferably, “haloaryl” refers to an aryl group wherein 1, 2 or 3 hydrogen atoms of the aryl group have been replaced by a halogen atom, most preferably fluoro. Preferred, yet non-limiting examples of haloaryl are 4-fluorophenyl, 2-chloro-4-fluorophenyl, 2,6-difluorophenyl, and 3,5-difluorophenyl.

The term “aryloxy” refers to an aryl group, as previously defined, attached to the parent molecular moiety via an oxygen atom. A preferred, yet non-limiting example of aryloxy is phenoxy.

The term “cycloalkyloxy” refers to a cycloalkyl group, as previously defined, attached to the parent molecular moiety via an oxygen atom. A preferred, yet non-limiting example of cycloalkyloxy is cyclopropoxy.

The term “heteroaryloxy” refers to a heteroaryl group, as previously defined, attached to the parent molecular moiety via an oxygen atom. A preferred, yet non-limiting example of heteroaryloxy is pyridyloxy (e.g., 2-pyridyloxy, 3-pyridyloxy or 4-pyridyloxy).

The term “heterocyclyloxy” refers to a heterocyclyl group, as previously defined, attached to the parent molecular moiety via an oxygen atom. A preferred, yet non-limiting example of heterocyclyloxy is azetidinyloxy.

The term “pharmaceutically acceptable salt” refers to those salts which retain the biological effectiveness and properties of the free bases or free acids, which are not biologically or otherwise undesirable. The salts are formed with inorganic acids such as hydrochloric acid, hydrobromic acid, sulfuric acid, nitric acid, phosphoric acid and the like, in particular hydrochloric acid, and organic acids such as acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, N-acetylcysteine and the like. In addition these salts may be prepared by addition of an inorganic base or an organic base to the free acid. Salts derived from an inorganic base include, but are not limited to, the sodium, potassium, lithium, ammonium, calcium, magnesium salts and the like. Salts derived from organic bases include, but are not limited to salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, lysine, arginine, N-ethylpiperidine, piperidine, polyimine resins and the like. Particular pharmaceutically acceptable salts of compounds of formula (I) are hydrochloride salts.

The term “protective group” (PG) denotes the group which selectively blocks a reactive site in a multifunctional compound such that a chemical reaction can be carried out selectively at another unprotected reactive site in the meaning conventionally associated with it in synthetic chemistry. Protective groups can be removed at the appropriate point. Exemplary protective groups are amino-protective groups, carboxy-protective groups or hydroxy-protective groups. Particular protective groups are the tert-butoxycarbonyl (Boc), benzyloxycarbonyl (Cbz), fluorenylmethoxycarbonyl (Fmoc) and benzyl (Bn). Further particular protective groups are the tert-butoxycarbonyl (Boc) and the fluorenylmethoxycarbonyl (Fmoc). More particular protective group is the tert-butoxycarbonyl (Boc). Exemplary protective groups and their application in organic synthesis are described, for example, in “Protective Groups in Organic Chemistry” by T. W. Greene and P. G. M. Wutts, 5th Ed., 2014, John Wiley & Sons, N. Y.

The term “urea forming reagent” refers to a chemical compound that is able to render a first amine to a species that will react with a second amine, thereby forming an urea derivative. Non-limiting examples of urea forming reagents include bis(trichloromethyl) carbonate, phosgene, trichloromethyl chloroformate, (4-nitrophenyl) carbonate and 1,1′-carbonyldiimidazole. The urea forming reagents described in G. Sartori et al., Green Chemistry 2000, 2, 140 are incorporated herein by reference.

The compounds of formula (I) can contain several asymmetric centers and can be present in the form of optically pure enantiomers, mixtures of enantiomers such as, for example, racemates, optically pure diastereoisomers, mixtures of diastereoisomers, diastereoisomeric racemates or mixtures of diastereoisomeric racemates. In a preferred embodiment, the compound of formula (I) according to the invention is a cis-enantiomer of formula (Ia) or (Ib), respectively, as described herein.

According to the Cahn-Ingold-Prelog Convention, the asymmetric carbon atom can be of the “R” or “S” configuration.

The abbreviation “MAGL” refers to the enzyme monoacylglycerol lipase. The terms “MAGL” and “monoacylglycerol lipase” are used herein interchangeably.

The term “treatment” as used herein includes: (1) inhibiting the state, disorder or condition (e.g. arresting, reducing or delaying the development of the disease, or a relapse thereof in case of maintenance treatment, of at least one clinical or subclinical symptom thereof); and/or (2) relieving the condition (i.e., causing regression of the state, disorder or condition or at least one of its clinical or subclinical symptoms). The benefit to a patient to be treated is either statistically significant or at least perceptible to the patient or to the physician. However, it will be appreciated that when a medicament is administered to a patient to treat a disease, the outcome may not always be effective treatment.

The term “prophylaxis” as used herein includes: preventing or delaying the appearance of clinical symptoms of the state, disorder or condition developing in a mammal and especially a human that may be afflicted with or predisposed to the state, disorder or condition but does not yet experience or display clinical or subclinical symptoms of the state, disorder or condition.

The term “neuroinflammation” as used herein relates to acute and chronic inflammation of the nervous tissue, which is the main tissue component of the two parts of the nervous system; the brain and spinal cord of the central nervous system (CNS), and the branching peripheral nerves of the peripheral nervous system (PNS). Chronic neuroinflammation is associated with neurodegenerative diseases such as Alzheimer's disease, Parkinson's disease and multiple sclerosis. Acute neuroinflammation usually follows injury to the central nervous system immediately, e.g., as a result of traumatic brain injury (TBI).

The term “traumatic brain injury” (“TBI”, also known as “intracranial injury”), relates to damage to the brain resulting from external mechanical force, such as rapid acceleration or deceleration, impact, blast waves, or penetration by a projectile.

The term “neurodegenerative diseases” relates to diseases that are related to the progressive loss of structure or function of neurons, including death of neurons. Examples of neurodegenerative diseases include, but are not limited to, multiple sclerosis, Alzheimer's disease, Parkinson's disease and amyotrophic lateral sclerosis.

The term “mental disorders” (also called mental illnesses or psychiatric disorders) relates to behavioral or mental patterns that may cause suffering or a poor ability to function in life. Such features may be persistent, relapsing and remitting, or occur as a single episode. Examples of mental disorders include, but are not limited to, anxiety and depression.

The term “pain” relates to an unpleasant sensory and emotional experience associated with actual or potential tissue damage. Examples of pain include, but are not limited to, nociceptive pain, chronic pain (including idiopathic pain), neuropathic pain including chemotherapy induced neuropathy, phantom pain and psychogenic pain. A particular example of pain is neuropathic pain, which is caused by damage or disease affecting any part of the nervous system involved in bodily feelings (i.e., the somatosensory system). In one embodiment, “pain” is neuropathic pain resulting from amputation or thoracotomy. In one embodiment, “pain” is chemotherapy induced neuropathy.

The term “neurotoxicity” relates to toxicity in the nervous system. It occurs when exposure to natural or artificial toxic substances (neurotoxins) alter the normal activity of the nervous system in such a way as to cause damage to nervous tissue. Examples of neurotoxicity include, but are not limited to, neurotoxicity resulting from exposure to substances used in chemotherapy, radiation treatment, drug therapies, drug abuse, and organ transplants, as well as exposure to heavy metals, certain foods and food additives, pesticides, industrial and/or cleaning solvents, cosmetics, and some naturally occurring substances.

The term “cancer” refers to a disease characterized by the presence of a neoplasm or tumor resulting from abnormal uncontrolled growth of cells (such cells being “cancer cells”). As used herein, the term cancer explicitly includes, but is not limited to, hepatocellular carcinoma, colon carcinogenesis and ovarian cancer.

The term “mammal” as used herein includes both humans and non-humans and includes but is not limited to humans, non-human primates, canines, felines, murines, bovines, equines, and porcines. In a particularly preferred embodiment, the term “mammal” refers to humans.

### Compounds of the Invention

In a first aspect, the present invention provides a compound of formula (I)

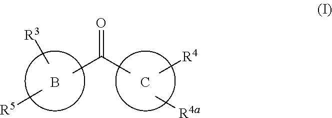

or a pharmaceutically acceptable salt thereof, wherein:
R⁵ is selected from a group

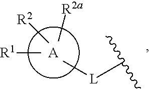

C₁₋₆-alkyl, C₁₋₆-alkoxy, halo-C₁₋₆-alkyl, halo-C₁₋₆-alkoxy, C₁₋₆-alkyl-SO₂—, C₁₋₆-alkyl-SO₂NH—;
R¹ is selected from hydrogen, halogen, SF₅, a group

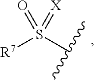

sulfamoyl, (C₁₋₆-alkyl)₂PO—C₁₋₆-alkyl-, a group

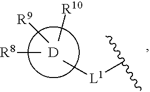

cyano, C₁₋₆-alkyl, C₁₋₆-alkoxy, halo-C₁₋₆-alkyl, halo-C₁₋₆-alkoxy, carbamoyl, oxo, C₁₋₆-alkyl-NHC(O)—, C₁₋₆-alkoxycarbonyl, and C₁₋₆-alkoxycarbonyl-C₁₋₆-alkyl-; C₃₋₁₀-cycloalkyl; and
R² is selected from hydrogen, halogen, cyano, C₁₋₆-alkyl, C₁₋₆-alkoxy, halo-C₁₋₆-alkyl, halo-C₁₋₆-alkoxy, and C₁₋₆-alkylsulfonyl; or
R¹ and R², taken together with the atom(s) to which they are attached, form a 3- to 14-membered heterocycle that is optionally substituted with one or more C₁₋₆-alkyl substituents;
R^{2a} is selected from hydrogen and halogen;
R³ is selected from hydrogen and C₁₋₆-alkyl;
R⁴ is selected from hydrogen, hydroxy, amino, a group

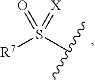

a group

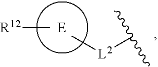

sulfamoyl, carboxy, hydroxy-C₁₋₆-alkyl, C₁₋₆-alkyl-SO₂NH—, carbamoyl, C₁₋₆-alkyl-NHC(O)—, (C₁₋₆-alkyl)₂NC(O)—, C₁₋₆-alkyl-C(O)NH—, C₁₋₆-alkoxy-C(O)NH—, and C₁₋₆-alkoxy-C(O)—;
R^{4a} is selected from hydrogen and C₁₋₆-alkyl;
R⁶ is selected from hydrogen, C₆₋₁₄-aryl and halo-C₆₋₁₄-aryl;
R⁷ is selected from C₁₋₆-alkyl, cyano-C₁₋₆-alkyl, and halo-C₁₋₆-alkyl;
R⁸ is selected from hydrogen, halogen, cyano, hydroxy, oxo, carboxy, C₁₋₆-alkoxycarbonyl, C₁₋₆-alkyl, a group

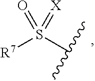

a group

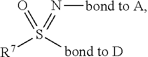

a group

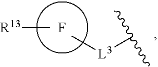

C₁₋₆-alkyl-SO—, C₁₋₆-alkyl-S—, C₁₋₆-alkyl-SO₂—C₁₋₆-alkyl-, (C₁₋₆-alkyl)₂-PO—, C₁₋₆-alkoxy, halo-C₁₋₆-alkyl, halo-C₁₋₆-alkoxy, carbamoyl, and C₁₋₆-alkyl-NHC(O)—;
R⁹ is selected from hydrogen, halogen, hydroxy, C₁₋₆-alkyl, and halo-C₁₋₆-alkyl;
R¹⁰ is selected from hydrogen and halogen;
R¹¹ is selected from hydrogen, methyl, cyclopropylmethyl, and phenyl;
R¹² is selected from hydrogen, halogen, oxo, C₁₋₆-alkyl, C₁₋₆-alkoxycarbonyl, and C₃₋₁₀-cycloalkyl;
R¹³ is selected from hydrogen, C₁₋₆-alkyl and halo-C₁₋₆-alkyl;
A is selected from C₆₋₁₄-aryl, C₃₋₁₀-cycloalkyl, 5- to 14-membered heteroaryl, and 3- to 14-membered heterocyclyl;
B is selected from azetidinyl, piperidyl, piperazinyl, 2-azaspiro[3.3]heptanyl, 2,6-diazaspiro[3.3]heptanyl, 2-azaspiro[3.4]octanyl, 6-azaspiro[3.4]octanyl, 2,7-diazaspiro[3.4]octanyl, 8-azabicyclo[3.2.1]octanyl, 2,7-diazaspiro[3.5]nonanyl, 2-azaspiro[3.5]nonanyl, 7-azaspiro[3.5]nonanyl, 1,2,3,3a,4,5,6,6a-octahydrocyclopenta[c]pyrrolyl, cyclobutyl, cubanyl, bicyclo[1.1.1]pentanyl, and spiro[3.3]heptanyl;
C is selected from phenyl, azetidinyl, pyrrolidinyl, piperidyl, piperazinyl, morpholinyl, 4,5-dihydroisoxazolyl, 2-azabicyclo[2.1.1]hexanyl, 2-azaspiro[3.3]heptanyl, and 2,8-diazaspiro[3.5]nonanyl;
D is selected from C₆₋₁₄-aryl, C₃₋₁₀-cycloalkyl, 5-14-membered heteroaryl, and 3- to 14-membered heterocyclyl;
E is selected from 5- to 14-membered heteroaryl, 3- to 14-membered heterocyclyl, and C₃₋₁₀-cycloalkyl;
F is selected from C₃₋₁₀-cycloalkyl, 3- to 14-membered heterocyclyl, and 5- to 14-membered heteroaryl;
L is selected from a covalent bond, carbonyl, —CHR⁶—, —O—, —NR¹¹—, —NHCH₂—, —CH₂NR¹¹—, —N(CH₃) CH₂—, —OCH₂—, —CH₂O—, —CH₂OCH₂—, —CH₂CH₂—, —CF₂CH₂—, —CH₂CF₂—, —CH═CH—, —C≡C—, —SO₂—, —SO₂NH—, —SO₂NHCH₂—, —C(O)NH—, —NHC(O)—, —CH₂NHC(O)—, and

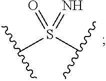 [SMILES: *S(*)(=N)=O]

L¹ is selected from a covalent bond, carbonyl, —CH₂—, —CH₂O—, —OCH₂—, —NHCH₂—, CH₂NH—, —NR¹¹—, —C(O)—NH—NH—C(O)—CH₂—, —SO₂—, and —O—;
L² is selected from a covalent bond and —SO₂—;
L³ is selected from a covalent bond, carbonyl, and —CH₂—; and
X is selected from O and NH.

In one embodiment, the present invention provides a compound of formula (I) as described herein, or a pharmaceutically acceptable salt thereof, wherein:
R⁵ is selected from a group

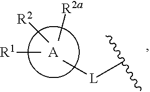

C₁₋₆-alkyl, C₁₋₆-alkoxy, halo-C₁₋₆-alkyl, halo-C₁₋₆-alkoxy, C₁₋₆-alkyl-SO₂—, C₁₋₆-alkyl-SO₂NH—;
R¹ is selected from hydrogen, halogen, SF₅, a group

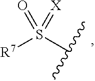

sulfamoyl, (C₁₋₆-alkyl)₂PO—C₁₋₆-alkyl-, a group

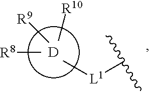

cyano, C₁₋₆-alkyl, C₁₋₆-alkoxy, halo-C₁₋₆-alkyl, halo-C₁₋₆-alkoxy, carbamoyl, oxo, C₁₋₆-alkyl-NHC(O)—, C₁₋₆-alkoxycarbonyl, and C₁₋₆-alkoxycarbonyl-C₁₋₆-alkyl-; C₃₋₁₀-cycloalkyl; and
R² is selected from hydrogen, halogen, cyano, C₁₋₆-alkyl, C₁₋₆-alkoxy, halo-C₁₋₆-alkyl, halo-C₁₋₆-alkoxy, and C₁₋₆-alkylsulfonyl; or
R¹ and R², taken together with the atom(s) to which they are attached, form a 3- to 14-membered heterocycle that is optionally substituted with one or more C₁₋₆-alkyl substituents;
R^{2a} is selected from hydrogen and halogen;
R³ is selected from hydrogen and C₁₋₆-alkyl;
R⁴ is selected from hydrogen, hydroxy, amino, a group

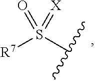

a group

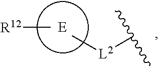

sulfamoyl, carboxy, hydroxy-C₁₋₆-alkyl, C₁₋₆-alkyl-SO₂NH—, carbamoyl, C₁₋₆-alkyl-NHC(O)—, (C₁₋₆-alkyl)₂NC(O)—, C₁₋₆-alkyl-C(O)NH—, C₁₋₆-alkoxy-C(O)NH—, and C₁₋₆-alkoxy-C(O)—;
R^{4a} is selected from hydrogen and C₁₋₆-alkyl;
R⁶ is selected from hydrogen, C₆₋₁₄-aryl and halo-C₆₋₁₄-aryl;
R⁷ is selected from C₁₋₆-alkyl, cyano-C₁₋₆-alkyl, and halo-C₁₋₆-alkyl;
R⁸ is selected from hydrogen, halogen, cyano, hydroxy, oxo, carboxy, C₁₋₆-alkoxycarbonyl, C₁₋₆-alkyl, a group

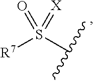

a group

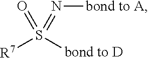

a group

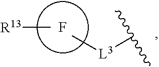

C₁₋₆-alkyl-SO—, C₁₋₆-alkyl-S—, C₁₋₆-alkyl-SO₂—C₁₋₆-alkyl-, (C₁₋₆-alkyl)₂-PO—, C₁₋₆-alkoxy, halo-C₁₋₆-alkyl, halo-C₁₋₆-alkoxy, carbamoyl, and C₁₋₆-alkyl-NHC(O)—;
R⁹ is selected from hydrogen, halogen, hydroxy, C₁₋₆-alkyl, and halo-C₁₋₆-alkyl;
R¹⁰ is selected from hydrogen and halogen;
R¹¹ is selected from hydrogen, methyl, cyclopropylmethyl, and phenyl;
R¹² is selected from hydrogen, halogen, oxo, C₁₋₆-alkyl, C₁₋₆-alkoxycarbonyl, and C₃₋₁₀-cycloalkyl;
R¹³ is selected from hydrogen, C₁₋₆-alkyl and halo-C₁₋₆-alkyl;
A is selected from C₆₋₁₄-aryl, C₃₋₁₀-cycloalkyl, 5- to 6-membered heteroaryl, and 3- to 14-membered heterocyclyl;
B is selected from azetidinyl, piperidyl, piperazinyl, 2-azaspiro[3.3]heptanyl, 2,6-diazaspiro[3.3]heptanyl, 2-azaspiro[3.4]octanyl, 6-azaspiro[3.4]octanyl, 2,7-diazaspiro[3.4]octanyl, 2,7-diazaspiro[3.5]nonanyl, 2-azaspiro[3.5]nonanyl, 7-azaspiro[3.5]nonanyl, 1,2,3,3a,4,5,6,6a-octahydrocyclopenta[c]pyrrolyl, cyclobutyl, cubanyl, spiro[3.3]heptanyl, a group

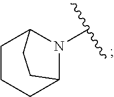 [SMILES: *N1C2CCCC1CC2]

and a group

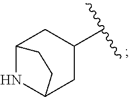 [SMILES: *C1CC2CCC(C1)N2]

C is selected from phenyl, azetidinyl, pyrrolidinyl, piperidyl, piperazinyl, morpholinyl, 4,5-dihydroisoxazolyl, 2-azabicyclo[2.1.1]hexanyl, 2-azaspiro[3.3]heptanyl, and 2,8-diazaspiro[3.5]nonanyl;
D is selected from C₆₋₁₄-aryl, C₃₋₁₀-cycloalkyl, 5-6-membered heteroaryl, and 3- to 14-membered heterocyclyl;
E is selected from 5- to 14-membered heteroaryl, 3- to 14-membered heterocyclyl, and C₃₋₁₀-cycloalkyl;
F is selected from C₃₋₁₀-cycloalkyl, 3- to 14-membered heterocyclyl, and 5- to 14-membered heteroaryl;
L is selected from a covalent bond, carbonyl, —CHR⁶—, —O—, —NR¹¹—, —NHCH₂—, —CH₂NR¹¹—, —N(CH₃) CH₂—, —OCH₂—, —CH₂O—, —CH₂OCH₂—, —CH₂CH₂—, —CF₂CH₂—, —CH₂CF₂—, —CH═CH—, —C≡C—, —SO₂—, —SO₂NH—, —SO₂NHCH₂—, —C(O)NH—, —NHC(O)—, —CH₂NHC(O)—, and

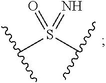 [SMILES: *S(*)(=N)=O]

L¹ is selected from a covalent bond, carbonyl, —CH₂—, —CH₂O—, —OCH₂—, —NHCH₂—, CH₂NH—, —NR¹¹—, —C(O)—NH—NH—C(O)—CH₂—, —SO₂—, and —O—;
L² is selected from a covalent bond and —SO₂—;
L³ is selected from a covalent bond, carbonyl, and —CH₂—;
X is selected from O and NH;
   provided that when B is 2-azaspiro[3.3]heptanyl or 2,6-diazaspiro[3.3]heptanyl, L is not a covalent bond.

In one embodiment, the present invention provides a compound of formula (I) as described herein, or a pharmaceutically acceptable salt thereof, wherein said compound of formula (I) is a compound of formula (I-I)

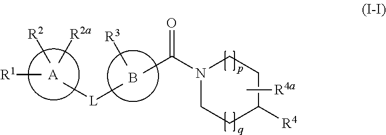

wherein R¹ to R⁴, R^{2a}, R^{4a}, A, B, and L are as defined herein; and
p and q are each independently selected from 0 and 1.

In one embodiment, the present invention provides a compound of formula (I) as described herein, or a pharmaceutically acceptable salt thereof, wherein said compound of formula (I) is a compound of formula (I-II)

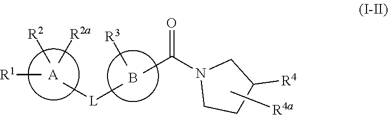

wherein R¹ to R⁴, R^{2a}, R^{4a}, A, B, and L are as defined herein.

In one embodiment, the present invention provides a compound of formula (I) as described herein, or a pharmaceutically acceptable salt thereof, wherein:
R⁵ is selected from a group

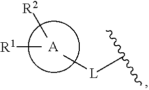

C₁₋₆-alkyl-SO₂—, C₁₋₆-alkyl-SO₂NH—, and halo-C₁₋₆-alkoxy;
R¹ is selected from hydrogen, halogen, SF₅, a group

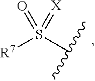

sulfamoyl, (C₁₋₆-alkyl)₂PO—C₁₋₆-alkyl-, a group

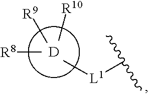

C₁₋₆-alkyl, C₁₋₆-alkoxy, halo-C₁₋₆-alkyl, halo-C₁₋₆-alkoxy, carbamoyl, oxo, C₁₋₆-alkyl-NHC(O)—, C₁₋₆-alkoxycarbonyl, and C₁₋₆-alkoxycarbonyl-C₁₋₆-alkyl-; and
R² is selected from hydrogen, halogen, cyano, C₁₋₆-alkyl, C₁₋₆-alkoxy, halo-C₁₋₆-alkyl, halo-C₁₋₆-alkoxy, and C₁₋₆-alkylsulfonyl; or
R¹ and R², taken together with the atom(s) to which they are attached, form a 3- to 14-membered heterocycle that is optionally substituted with 1-2 C₁₋₆-alkyl substituents;
R^{2a} is selected from hydrogen and halogen;
R⁶ is selected from hydrogen, C₆₋₁₄-aryl, and halo-C₆₋₁₄-aryl;
R⁷ is selected from C₁₋₆-alkyl, cyano-C₁₋₆-alkyl, and halo-C₁₋₆-alkyl;
R⁸ is selected from hydrogen, halogen, cyano, hydroxy, oxo, carboxy, C₁₋₆-alkoxycarbonyl, C₁₋₆-alkyl, a group

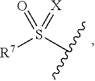

a group

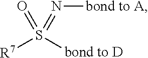

a group

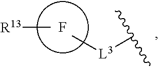

C₁₋₆-alkyl-SO—, C₁₋₆-alkyl-S—, C₁₋₆-alkyl-SO₂—C₁₋₆-alkyl-, (C₁₋₆-alkyl)₂-PO—, C₁₋₆-alkoxy, halo-C₁₋₆-alkyl, halo-C₁₋₆-alkoxy, carbamoyl, and C₁₋₆-alkyl-NHC(O)—;
R⁹ is selected from hydrogen, halogen, hydroxy, C₁₋₆-alkyl, and halo-C₁₋₆-alkyl;
R¹⁰ is selected from hydrogen and halogen;
R¹¹ is selected from hydrogen, methyl, cyclopropylmethyl, and phenyl;
R¹³ is selected from hydrogen, C₁₋₆-alkyl and halo-C₁₋₆-alkyl;
A is selected from C₆₋₁₄-aryl, C₃₋₁₀-cycloalkyl, 3- to 14-membered heterocyclyl, and 5- to 14-membered heteroaryl;
D is selected from C₆₋₁₄-aryl, C₃₋₁₀-cycloalkyl, 5-14-membered heteroaryl, and 3- to 14-membered heterocyclyl;
F is selected from C₃₋₁₀-cycloalkyl, 3- to 14-membered heterocyclyl, and 5- to 14-membered heteroaryl;
L is selected from a covalent bond, —CHR⁶—, —O—, —NR¹¹—, —NHCH₂—, —CH₂NR¹¹—, —OCH₂—, —CH₂O—, —CH₂OCH₂—, —CH₂CH₂—, —CF₂CH₂—, —CH₂CF₂—, —CH═CH—, —C≡C—, —SO₂—, —SO₂NH—, —SO₂NHCH₂—, —C(O)NH—, —NHC(O)—, —CH₂NHC(O)—, and

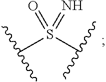 [SMILES: *S(*)(=N)=O]

L¹ is selected from a covalent bond, carbonyl, —CH₂—, —CH₂O—, —NHCH₂—, CH₂NH—, —NR¹¹—, —C(O)—NH—NH—C(O)—CH₂—, —SO₂—, and —O—;
L³ is selected from a covalent bond, carbonyl, and —CH₂—; and
X is selected from O and NH.

In one embodiment, the present invention provides a compound of formula (I) as described herein, or a pharmaceutically acceptable salt thereof, wherein:
R⁵ is selected from a group

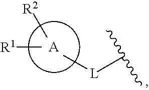

C₁₋₆-alkyl-SO₂—, C₁₋₆-alkyl-SO₂NH—, and halo-C₁₋₆-alkoxy;
R¹ is selected from hydrogen, halogen, SF₅, a group

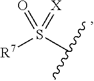

sulfamoyl, (C₁₋₆-alkyl)₂PO—C₁₋₆-alkyl-, a group

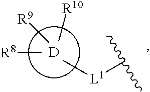

C₁₋₆-alkyl, C₁₋₆-alkoxy, halo-C₁₋₆-alkyl, halo-C₁₋₆-alkoxy, carbamoyl, oxo, C₁₋₆-alkyl-NHC(O)—, C₁₋₆-alkoxycarbonyl, and C₁₋₆-alkoxycarbonyl-C₁₋₆-alkyl-; and
R² is selected from hydrogen, halogen, cyano, C₁₋₆-alkyl, C₁₋₆-alkoxy, halo-C₁₋₆-alkyl, halo-C₁₋₆-alkoxy, and C₁₋₆-alkylsulfonyl; or
R¹ and R², taken together with the atom(s) to which they are attached, form a 3- to 14-membered heterocycle that is optionally substituted with 1-2 C₁₋₆-alkyl substituents;
R^{2a} is selected from hydrogen and halogen;
R⁶ is selected from hydrogen, C₆₋₁₄-aryl, and halo-C₆₋₁₄-aryl;
R⁷ is selected from C₁₋₆-alkyl, cyano-C₁₋₆-alkyl, and halo-C₁₋₆-alkyl;
R⁸ is selected from hydrogen, halogen, cyano, hydroxy, oxo, carboxy, C₁₋₆-alkoxycarbonyl, C₁₋₆-alkyl, a group

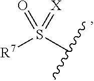

a group

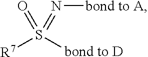

a group

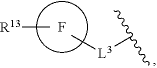

C₁₋₆-alkyl-SO—, C₁₋₆-alkyl-S—, C₁₋₆-alkyl-SO₂—C₁₋₆-alkyl-, (C₁₋₆-alkyl)₂-PO—, C₁₋₆-alkoxy, halo-C₁₋₆-alkyl, halo-C₁₋₆-alkoxy, carbamoyl, and C₁₋₆-alkyl-NHC(O)—;
R⁹ is selected from hydrogen, halogen, hydroxy, C₁₋₆-alkyl, and halo-C₁₋₆-alkyl;
R¹⁰ is selected from hydrogen and halogen;
R¹¹ is selected from hydrogen, methyl, cyclopropylmethyl, and phenyl;
R¹³ is selected from hydrogen, C₁₋₆-alkyl and halo-C₁₋₆-alkyl;
A is selected from C₆₋₁₄-aryl, C₃₋₁₀-cycloalkyl, 3- to 14-membered heterocyclyl, and 5- to 6-membered heteroaryl;
D is selected from C₆₋₁₄-aryl, C₃₋₁₀-cycloalkyl, 5-6-membered heteroaryl, and 3- to 14-membered heterocyclyl;
F is selected from C₃₋₁₀-cycloalkyl, 3- to 14-membered heterocyclyl, and 5- to 14-membered heteroaryl;
L is selected from a covalent bond, —CHR⁶—, —O—, —NR¹¹—, —NHCH₂—, —CH₂NR¹¹—, —OCH₂—, —CH₂O—, —CH₂OCH₂—, —CH₂CH₂—, —CF₂CH₂—, —CH₂CF₂—, —CH—CH—, —C≡C—, —SO₂—, —SO₂NH—, —SO₂NHCH₂—, —C(O)NH—, —NHC(O)—, —CH₂NHC(O)—, and

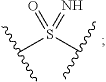 [SMILES: *S(*)(=N)=O]

L¹ is selected from a covalent bond, carbonyl, —CH₂—, —CH₂O—, —NHCH₂—, CH₂NH—, —NR¹¹—, —C(O)—NH—NH—C(O)—CH₂—, —SO₂—, and —O—;
L³ is selected from a covalent bond, carbonyl, and —CH₂—; and
X is selected from O and NH.

In a preferred embodiment, the present invention provides a compound of formula (I) as described herein, or a pharmaceutically acceptable salt thereof, wherein:
R⁵ is a group

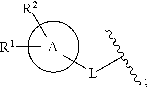

R¹ is selected from halogen, C₁₋₆-alkyl, halo-C₁₋₆-alkyl, and a group

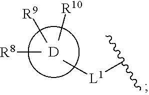

R² is selected from hydrogen, halogen, and halo-C₁₋₆-alkyl;
R^{2a} is hydrogen;
R⁸ and R′ are each independently selected from halogen and halo-C₁₋₆-alkyl;
R¹⁰ is hydrogen;
A is selected from C₆₋₁₄-aryl and C₃₋₁₀-cycloalkyl;
D is selected from C₆₋₁₄-aryl and C₃₋₁₀-cycloalkyl;
L is selected from a covalent bond, —O— and —CH₂O—; and is a covalent bond.

In a particularly preferred embodiment, the present invention provides a compound of formula (I) as described herein, or a pharmaceutically acceptable salt thereof, wherein:
R⁵ is a group

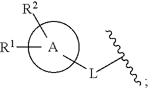

R¹ is selected from fluoro, chloro, tert-butyl, CHF₂, CF₃, and a group

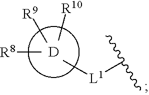

R² is selected from hydrogen, fluoro, chloro, and CF₃;
R^{2a} is hydrogen;
R⁸ and R⁹ are each independently selected from fluoro, chloro and CF₃;
R¹⁰ is hydrogen;
A is selected from phenyl and cyclopropyl;
D is selected from phenyl and cyclopropyl;
L is selected from a covalent bond, —O— and —CH₂O—; and
L¹ is a covalent bond.

In a preferred embodiment, the present invention provides a compound of formula (I) as described herein, or a pharmaceutically acceptable salt thereof, wherein:
B is selected from spiro[3.3]heptan-2-yl, azetidinyl, and 7-azaspiro[3.5]nonan-7-yl; and
R³ is hydrogen.

In a preferred embodiment, the present invention provides a compound of formula (I) as described herein, or a pharmaceutically acceptable salt thereof, wherein:
R⁴ is selected from a group

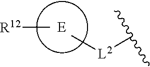

and carbamoyl;
R^{4a} is hydrogen;
R¹² is selected from hydrogen and oxo;
C is pyrrolidinyl;
E is selected from 5-membered heteroaryl and 5-membered heterocyclyl; and
L² is a covalent bond.

In a particularly preferred embodiment, the present invention provides a compound of formula (I) as described herein, or a pharmaceutically acceptable salt thereof, wherein:
R⁴ is selected from a group

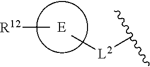

and carbamoyl;
R^{4a} is hydrogen;
R¹² is selected from hydrogen and oxo;
C is pyrrolidinyl;
E is selected from imidazolyl, pyrazolyl, triazolyl, and oxazolidinyl; and
L² is a covalent bond.

In one embodiment, the present invention provides a compound of formula (I) as described herein, or a pharmaceutically acceptable salt thereof, wherein:
R⁵ is selected from a group

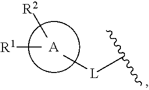

C₁₋₆-alkyl-SO₂—, C₁₋₆-alkyl-SO₂NH—, and halo-C₁₋₆-alkoxy;
R¹ is selected from hydrogen, halogen, SF₅, a group

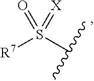

sulfamoyl, (C₁₋₆-alkyl)₂PO—C₁₋₆-alkyl-, a group

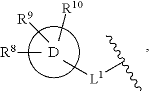

C₁₋₆-alkyl, C₁₋₆-alkoxy, halo-C₁₋₆-alkyl, halo-C₁₋₆-alkoxy, carbamoyl, oxo, C₁₋₆-alkyl-NHC(O)—, C₁₋₆-alkoxycarbonyl, and C₁₋₆-alkoxycarbonyl-C₁₋₆-alkyl-; and
R² is selected from hydrogen, halogen, cyano, C₁₋₆-alkyl, C₁₋₆-alkoxy, halo-C₁₋₆-alkyl, halo-C₁₋₆-alkoxy, and C₁₋₆-alkylsulfonyl; or
R¹ and R², taken together with the atom(s) to which they are attached, form a 3- to 14-membered heterocycle that is optionally substituted with 1-2 C₁₋₆-alkyl substituents;
R^{2a} is selected from hydrogen and halogen;
R³ is selected from hydrogen and C₁₋₆-alkyl;
R⁴ is selected from hydrogen, hydroxy, amino, a group

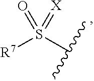

a group

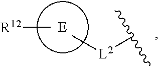

sulfamoyl, carboxy, hydroxy-C₁₋₆-alkyl, C₁₋₆-alkyl-SO₂NH—, carbamoyl, C₁₋₆-alkyl-NHC(O)—, (C₁₋₆-alkyl)₂NC(O)—, C₁₋₆-alkyl-C(O)NH—, C₁₋₆-alkoxy-C(O)NH—, and C₁₋₆-alkoxy-C(O)—;
R^{4a} is selected from hydrogen and C₁₋₆-alkyl;
R⁶ is selected from hydrogen, C₆₋₁₄-aryl, and halo-C₆₋₁₄-aryl;
R⁷ is selected from C₁₋₆-alkyl, cyano-C₁₋₆-alkyl, and halo-C₁₋₆-alkyl;
R⁸ is selected from hydrogen, halogen, cyano, hydroxy, oxo, carboxy, C₁₋₆-alkoxycarbonyl, C₁₋₆-alkyl, a group

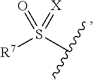

a group

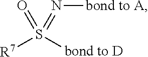

a group

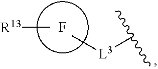

C₁₋₆-alkyl-SO—, C₁₋₆-alkyl-S—, C₁₋₆-alkyl-SO₂—C₁₋₆-alkyl-, (C₁₋₆-alkyl)₂-PO—, C₁₋₆-alkoxy, halo-C₁₋₆-alkyl, halo-C₁₋₆-alkoxy, carbamoyl, and C₁₋₆-alkyl-NHC(O)—;
R⁹ is selected from hydrogen, halogen, hydroxy, C₁₋₆-alkyl, and halo-C₁₋₆-alkyl;
R¹⁰ is selected from hydrogen and halogen;
R¹¹ is selected from hydrogen, methyl, cyclopropylmethyl, and phenyl;
R¹² is selected from hydrogen, halogen, oxo, C₁₋₆-alkyl, C₁₋₆-alkoxycarbonyl, and C₃₋₁₀-cycloalkyl;
R¹³ is selected from hydrogen, C₁₋₆-alkyl and halo-C₁₋₆-alkyl;
A is selected from C₆₋₁₄-aryl, C₃₋₁₀-cycloalkyl, 3- to 14-membered heterocyclyl, and 5- to 14-membered heteroaryl;
B is selected from azetidinyl, piperidyl, piperazinyl, 2-azaspiro[3.3]heptanyl, 2,6-diazaspiro[3.3]heptanyl, 2-azaspiro[3.4]octanyl, 6-azaspiro[3.4]octanyl, 2,7-diazaspiro[3.4]octanyl, 8-azabicyclo[3.2.1]octanyl, 2,7-diazaspiro[3.5]nonanyl, 2-azaspiro[3.5]nonanyl, 7-azaspiro[3.5]nonanyl, 1,2,3,3a,4,5,6,6a-octahydrocyclopenta[c]pyrrolyl, cyclobutyl, cubanyl, bicyclo[1.1.1]pentanyl, and spiro[3.3]heptanyl;
C is selected from phenyl, azetidinyl, pyrrolidinyl, piperidyl, piperazinyl, morpholinyl, 4,5-dihydroisoxazolyl, 2-azabicyclo[2.1.1]hexanyl, 2-azaspiro[3.3]heptanyl, and 2,8-diazaspiro[3.5]nonanyl;
D is selected from C₆₋₁₄-aryl, C₃₋₁₀-cycloalkyl, 5-14-membered heteroaryl, and 3- to 14-membered heterocyclyl;
E is selected from 5- to 14-membered heteroaryl, 3- to 14-membered heterocyclyl, and C₃₋₁₀-cycloalkyl;
F is selected from C₃₋₁₀-cycloalkyl, 3- to 14-membered heterocyclyl, and 5- to 14-membered heteroaryl;
L is selected from a covalent bond, —CHR⁶—, —O—, —NR¹¹—, —NHCH₂—, —CH₂NR¹¹—, —OCH₂—, —CH₂O—, —CH₂OCH₂—, —CH₂CH₂—, —CF₂CH₂—, —CH₂CF₂—, —CH—CH—, —C≡C—, —SO₂—, —SO₂NH—, —SO₂NHCH₂—, C(O)NH—, —NHC(O)—, —CH₂NHC(O)—, and

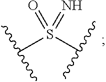 [SMILES: *S(*)(=N)=O]

L¹ is selected from a covalent bond, carbonyl, CH₂—, CH₂O—, —NHCH₂—, CH₂NH—, —NR¹¹—, —C(O)—NH—NH—C(O)—CH₂—, —SO₂—, and —O—;
L² is selected from a covalent bond and —SO₂—;
L³ is selected from a covalent bond, carbonyl, and —CH₂—; and
X is selected from O and NH.
In one embodiment, the present invention provides a compound of formula (I) as described herein, or a pharmaceutically acceptable salt thereof, wherein:
R⁵ is selected from a group

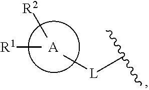

C₁₋₆-alkyl-SO₂—, C₁₋₆-alkyl-SO₂NH—, and halo-C₁₋₆-alkoxy;
R¹ is selected from hydrogen, halogen, SF₅, a group

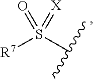

sulfamoyl, (C₁₋₆-alkyl)₂PO—C₁₋₆-alkyl-, a group

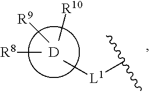

C₁₋₆-alkyl, C₁₋₆-alkoxy, halo-C₁₋₆-alkyl, halo-C₁₋₆-alkoxy, carbamoyl, oxo, C₁₋₆-alkyl-NHC(O)—, C₁₋₆-alkoxycarbonyl, and C₁₋₆-alkoxycarbonyl-C₁₋₆-alkyl-; and
R² is selected from hydrogen, halogen, cyano, C₁₋₆-alkyl, C₁₋₆-alkoxy, halo-C₁₋₆-alkyl, halo-C₁₋₆-alkoxy, and C₁₋₆-alkylsulfonyl; or
R¹ and R², taken together with the atom(s) to which they are attached, form a 3- to 14-membered heterocycle that is optionally substituted with 1-2 C₁₋₆-alkyl substituents;
R^{2a} is selected from hydrogen and halogen;
R³ is selected from hydrogen and C₁₋₆-alkyl;
R⁴ is selected from hydrogen, hydroxy, amino, a group

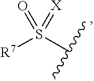

a group

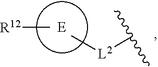

sulfamoyl, carboxy, hydroxy-C₁₋₆-alkyl, C₁₋₆-alkyl-SO₂NH—, carbamoyl, C₁₋₆-alkyl-NHC(O)—, (C₁₋₆-alkyl)₂NC(O)—, C₁₋₆-alkyl-C(O)NH—C₁₋₆-alkoxy-C(O)NH—, and C₁₋₆-alkoxy-C(O)—;
R^{4a} is selected from hydrogen and C₁₋₆-alkyl;
R⁶ is selected from hydrogen, C₆₋₁₄-aryl, and halo-C₆₋₁₄-aryl;
R⁷ is selected from C₁₋₆-alkyl, cyano-C₁₋₆-alkyl, and halo-C₁₋₆-alkyl;
R⁸ is selected from hydrogen, halogen, cyano, hydroxy, oxo, carboxy, C₁₋₆-alkoxycarbonyl, C₁₋₆-alkyl, a group

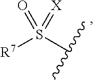

a group

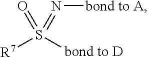

a group

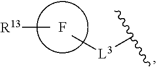

C₁₋₆-alkyl-SO—, C₁₋₆-alkyl-S—, C₁₋₆-alkyl-SO₂—C₁₋₆-alkyl-, (C₁₋₆-alkyl)₂-PO—, C₁₋₆-alkoxy, halo-C₁₋₆-alkyl, halo-C₁₋₆-alkoxy, carbamoyl, and C₁₋₆-alkyl-NHC(O)—;
R⁹ is selected from hydrogen, halogen, hydroxy, C₁₋₆-alkyl, and halo-C₁₋₆-alkyl;
R¹⁰ is selected from hydrogen and halogen;
R¹¹ is selected from hydrogen, methyl, cyclopropylmethyl, and phenyl;
R¹² is selected from hydrogen, halogen, oxo, C₁₋₆-alkyl, C₁₋₆-alkoxycarbonyl, and C₃₋₁₀-cycloalkyl;
R¹³ is selected from hydrogen, C₁₋₆-alkyl and halo-C₁₋₆-alkyl;
A is selected from C₆₋₁₄-aryl, C₃₋₁₀-cycloalkyl, 3- to 14-membered heterocyclyl, and 5- to 6-membered heteroaryl;
B is selected from azetidinyl, piperidyl, piperazinyl, 2-azaspiro[3.3]heptanyl, 2,6-diazaspiro[3.3]heptanyl, 2-azaspiro[3.4]octanyl, 6-azaspiro[3.4]octanyl, 2,7-diazaspiro[3.4]octanyl, 2,7-diazaspiro[3.5]nonanyl, 2-azaspiro[3.5]nonanyl, 7-azaspiro[3.5]nonanyl, 1,2,3,3a,4,5,6,6a-octahydrocyclopenta[c]pyrrolyl, cyclobutyl, cubanyl, spiro[3.3]heptanyl, a group

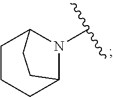 [SMILES: *N1C2CCCC1CC2]

and a group

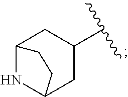 [SMILES: *C1CC2CCC(C1)N2]

C is selected from phenyl, azetidinyl, pyrrolidinyl, piperidyl, piperazinyl, morpholinyl, 4,5-dihydroisoxazolyl, 2-azabicyclo[2.1.1]hexanyl, 2-azaspiro[3.3]heptanyl, and 2,8-diazaspiro[3.5]nonanyl;
D is selected from C₆₋₁₄-aryl, C₃₋₁₀-cycloalkyl, 5-6-membered heteroaryl, and 3- to 14-membered heterocyclyl;
E is selected from 5- to 14-membered heteroaryl, 3- to 14-membered heterocyclyl, and C₃₋₁₀-cycloalkyl;
F is selected from C₃₋₁₀-cycloalkyl, 3- to 14-membered heterocyclyl, and 5- to 14-membered heteroaryl;
L is selected from a covalent bond, —CHR⁶—, —O—, —NR¹¹—, —NHCH₂—, —CH₂NR¹¹—, —OCH₂—, —CH₂O—, —CH₂OCH₂—, —CH₂CH₂—, —CF₂CH₂—, —CH₂CF₂—, —CH═CH—, —C≡C—, —SO₂—, —SO₂NH—, —SO₂NHCH₂—, —C(O)NH—, —NHC(O)—, —CH₂NHC(O)—, and

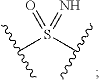 [SMILES: *S(*)(=N)=O]

L¹ is selected from a covalent bond, carbonyl, —CH₂—, —CH₂O—, —NHCH₂—, CH₂NH—, —NR¹¹—, —C(O)—NH—NH—C(O)—CH₂—, —SO₂—, and —O—;
L² is selected from a covalent bond and —SO₂—;
L³ is selected from a covalent bond, carbonyl, and —CH₂—; and
X is selected from O and NH.

In a preferred embodiment, the present invention provides a compound of formula (I) as described herein, or a pharmaceutically acceptable salt thereof, wherein:
R⁵ is a group

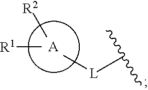

R¹ is selected from halogen, C₁₋₆-alkyl, halo-C₁₋₆-alkyl, and a group

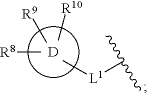

R² is selected from hydrogen, halogen, and halo-C₁₋₆-alkyl;
R^{2a} is hydrogen;
R³ is hydrogen;
R⁴ is selected from a group

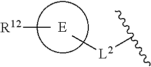

and carbamoyl;
R^{4a} is hydrogen;
R⁸ and R⁹ are each independently selected from halogen and halo-C₁₋₆-alkyl;
R¹⁰ is hydrogen;
R¹² is selected from hydrogen and oxo;
A is selected from C₆₋₁₄-aryl and C₃₋₁₀-cycloalkyl;
B is selected from spiro[3.3]heptan-2-yl, azetidinyl, and 7-azaspiro[3.5]nonan-7-yl;
C is pyrrolidinyl;
D is selected from C₆₋₁₄-aryl and C₃₋₁₀-cycloalkyl;
E is selected from 5-membered heteroaryl and 5-membered heterocyclyl;
L is selected from a covalent bond, —O— and —CH₂O—;
L¹ is a covalent bond; and
L² is a covalent bond.

In a particularly preferred embodiment, the present invention provides a compound of formula (I) as described herein, or a pharmaceutically acceptable salt thereof, wherein:
R⁵ is a group

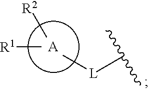

R¹ is selected from fluoro, chloro, tert-butyl, CHF₂, CF₃, and a group

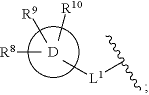

R² is selected from hydrogen, fluoro, chloro, and CF₃;
R^{2a} is hydrogen;
R³ is hydrogen;
R⁴ is selected from a group

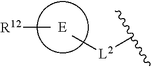

and carbamoyl;
R^{4a} is hydrogen;
R⁸ and R⁹ are each independently selected from fluoro, chloro and CF₃;
R¹⁰ is hydrogen;
R¹² is selected from hydrogen and oxo;
A is selected from phenyl and cyclopropyl;
B is selected from spiro[3.3]heptan-2-yl, azetidinyl, and 7-azaspiro[3.5]nonan-7-yl;
C is pyrrolidinyl;
D is selected from phenyl and cyclopropyl;
E is selected from imidazolyl, pyrazolyl, triazolyl, and oxazolidinyl;
L is selected from a covalent bond, —O— and —CH₂O—;
L¹ is a covalent bond; and
L² is a covalent bond.

In one embodiment, the present invention provides a compound of formula (I) as described herein, or a pharmaceutically acceptable salt thereof, wherein R⁵ is selected from a group

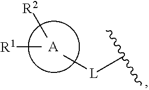

C₁₋₆-alkyl-SO₂—, C₁₋₆-alkyl-SO₂NH—, and halo-C₁₋₆-alkoxy; wherein R¹, R², A and L are as described herein.

In one embodiment, the present invention provides a compound of formula (I) as described herein, or a pharmaceutically acceptable salt thereof, wherein R¹ is selected from hydrogen, halogen, SF₅, a group

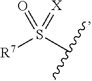

sulfamoyl, (C₁₋₆-alkyl)₂PO—C₁₋₆-alkyl-, a group

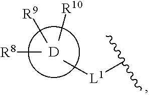

C₁₋₆-alkyl, C₁₋₆-alkoxy, halo-C₁₋₆-alkyl, halo-C₁₋₆-alkoxy, carbamoyl, oxo, C₁₋₆-alkyl-NHC(O)—, C₁₋₆-alkoxycarbonyl, and C₁₋₆-alkoxycarbonyl-C₁₋₆-alkyl-; wherein R⁷ to R¹⁰, X, D and L¹ are as defined herein.

In one embodiment, the present invention provides a compound of formula (I) as described herein, or a pharmaceutically acceptable salt thereof, wherein R¹ and R², taken together with the atom(s) to which they are attached, form a 3- to 14-membered heterocycle that is optionally substituted with one or more C₁₋₆-alkyl substituents.

In one embodiment, the present invention provides a compound of formula (I) as described herein, or a pharmaceutically acceptable salt thereof, wherein L is selected from a covalent bond, —CHR⁶—, —O—, —NR¹¹—, —NHCH₂—, —CH₂NR¹¹—, —OCH₂—, —CH₂O—, —CH₂OCH₂—, —CH₂CH₂—, —CF₂CH₂—, —CH₂CF₂—, —CH═CH—, —C≡C—, —SO₂—, —SO₂NH—, —SO₂NHCH₂—, —C(O)NH—, —NHC(O)—, —CH₂NHC(O)—, and

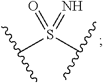

wherein R⁶ and R¹¹ are as defined herein.

In one embodiment, the present invention provides a compound of formula (I) as described herein, or a pharmaceutically acceptable salt thereof, wherein B is azetidinyl.

In one embodiment, the present invention provides a compound of formula (I) as described herein, or a pharmaceutically acceptable salt thereof, wherein B is piperidyl.

In one embodiment, the present invention provides a compound of formula (I) as described herein, or a pharmaceutically acceptable salt thereof, wherein B is piperazinyl.

In one embodiment, the present invention provides a compound of formula (I) as described herein, or a pharmaceutically acceptable salt thereof, wherein B is 2-azaspiro[3.3]heptanyl.

In one embodiment, the present invention provides a compound of formula (I) as described herein, or a pharmaceutically acceptable salt thereof, wherein B is 2,6-diazaspiro[3.3]heptanyl.

In one embodiment, the present invention provides a compound of formula (I) as described herein, or a pharmaceutically acceptable salt thereof, wherein B is 2-azaspiro[3.4]octanyl.

In one embodiment, the present invention provides a compound of formula (I) as described herein, or a pharmaceutically acceptable salt thereof, wherein B is 6-azaspiro[3.4]octanyl.

In one embodiment, the present invention provides a compound of formula (I) as described herein, or a pharmaceutically acceptable salt thereof, wherein B is 2,7-diazaspiro[3.4]octanyl.

In one embodiment, the present invention provides a compound of formula (I) as described herein, or a pharmaceutically acceptable salt thereof, wherein B is 8-azabicyclo[3.2.1]octanyl.

In one embodiment, the present invention provides a compound of formula (I) as described herein, or a pharmaceutically acceptable salt thereof, wherein B is 2,7-diazaspiro[3.5]nonanyl.

In one embodiment, the present invention provides a compound of formula (I) as described herein, or a pharmaceutically acceptable salt thereof, wherein B is 2-azaspiro[3.5]nonanyl.

In one embodiment, the present invention provides a compound of formula (I) as described herein, or a pharmaceutically acceptable salt thereof, wherein B is 7-azaspiro[3.5]nonanyl.

In one embodiment, the present invention provides a compound of formula (I) as described herein, or a pharmaceutically acceptable salt thereof, wherein B is 1,2,3,3a,4,5,6,6a-octahydrocyclopenta[c]pyrrolyl.

In one embodiment, the present invention provides a compound of formula (I) as described herein, or a pharmaceutically acceptable salt thereof, wherein B is cyclobutyl.

In one embodiment, the present invention provides a compound of formula (I) as described herein, or a pharmaceutically acceptable salt thereof, wherein B is cubanyl.

In one embodiment, the present invention provides a compound of formula (I) as described herein, or a pharmaceutically acceptable salt thereof, wherein B is bicyclo[1.1.1]pentanyl.

In one embodiment, the present invention provides a compound of formula (I) as described herein, or a pharmaceutically acceptable salt thereof, wherein B is spiro[3.3]heptanyl.

In one embodiment, the present invention provides a compound of formula (I) as described herein, or a pharmaceutically acceptable salt thereof, wherein C is phenyl.

In one embodiment, the present invention provides a compound of formula (I) as described herein, or a pharmaceutically acceptable salt thereof, wherein C is azetidinyl.

In one embodiment, the present invention provides a compound of formula (I) as described herein, or a pharmaceutically acceptable salt thereof, wherein C is pyrrolidinyl.

In one embodiment, the present invention provides a compound of formula (I) as described herein, or a pharmaceutically acceptable salt thereof, wherein C is piperidyl.

In one embodiment, the present invention provides a compound of formula (I) as described herein, or a pharmaceutically acceptable salt thereof, wherein C is piperazinyl.

In one embodiment, the present invention provides a compound of formula (I) as described herein, or a pharmaceutically acceptable salt thereof, wherein C is morpholinyl.

In one embodiment, the present invention provides a compound of formula (I) as described herein, or a pharmaceutically acceptable salt thereof, wherein C is 4,5-dihydroisoxazolyl.

In one embodiment, the present invention provides a compound of formula (I) as described herein, or a pharmaceutically acceptable salt thereof, wherein C is 2-azabicyclo[2.1.1]hexanyl.

In one embodiment, the present invention provides a compound of formula (I) as described herein, or a pharmaceutically acceptable salt thereof, wherein C is 2-azaspiro[3.3]heptanyl.

In one embodiment, the present invention provides a compound of formula (I) as described herein, or a pharmaceutically acceptable salt thereof, wherein C is 2,8-diazaspiro[3.5]nonanyl.

In one embodiment, the present invention provides a compound of formula (I) as described herein, or a pharmaceutically acceptable salt thereof, wherein B is azetidinyl and C is pyrrolidinyl.

In one embodiment, the present invention provides a compound of formula (I) as described herein, or a pharmaceutically acceptable salt thereof, wherein B is 2-azaspiro[3.3]heptanyl and C is pyrrolidinyl.

In one embodiment, the present invention provides a compound of formula (I) as described herein, or a pharmaceutically acceptable salt thereof, wherein B is 2,6-diazaspiro[3.3]heptanyl and C is pyrrolidinyl.

In one embodiment, the present invention provides a compound of formula (I) as described herein, or a pharmaceutically acceptable salt thereof, wherein B is 2,7-diazaspiro[3.5]nonanyl and C is pyrrolidinyl.

In one embodiment, the present invention provides a compound of formula (I) as described herein, or a pharmaceutically acceptable salt thereof, wherein B is 2-azaspiro[3.5]nonanyl and C is pyrrolidinyl.

In one embodiment, the present invention provides a compound of formula (I) as described herein, or a pharmaceutically acceptable salt thereof, wherein B is 7-azaspiro[3.5]nonanyl and C is pyrrolidinyl.

In one embodiment, the present invention provides a compound of formula (I) as described herein, or a pharmaceutically acceptable salt thereof, wherein B is 2-azaspiro[3.4]octanyl and C is pyrrolidinyl.

In one embodiment, the present invention provides a compound of formula (I) as described herein, or a pharmaceutically acceptable salt thereof, wherein B is 2,7-diazaspiro[3.4]octanyl and C is pyrrolidinyl.

In one embodiment, the present invention provides a compound of formula (I) as described herein, or a pharmaceutically acceptable salt thereof, wherein B is piperidyl and C is pyrrolidinyl.

In one embodiment, the present invention provides a compound of formula (I) as described herein, or a pharmaceutically acceptable salt thereof, wherein B and C are both azetidinyl.

In one embodiment, the present invention provides a compound of formula (I) as described herein, or a pharmaceutically acceptable salt thereof, wherein B is 2,7-diazaspiro[3.5]nonanyl and C is azetidinyl.

In one embodiment, the present invention provides a compound of formula (I) as described herein, or a pharmaceutically acceptable salt thereof, wherein B is azetidinyl and C is morpholinyl.

In one embodiment, the present invention provides a compound of formula (I) as described herein, or a pharmaceutically acceptable salt thereof, wherein B is azetidinyl and C is piperidyl.

In one embodiment, the present invention provides a compound of formula (I) as described herein, or a pharmaceutically acceptable salt thereof, wherein B is azetidinyl and C is piperazinyl.

In one embodiment, the present invention provides a compound of formula (I) as described herein, or a pharmaceutically acceptable salt thereof, wherein B is 2-azaspiro[3.3]heptanyl and C is piperidyl.

In one embodiment, the present invention provides a compound of formula (I) as described herein, or a pharmaceutically acceptable salt thereof, wherein B is azetidinyl and C is 4,5-dihydroisoxazolyl.

In one embodiment, the present invention provides a compound of formula (I) as described herein, or a pharmaceutically acceptable salt thereof, wherein B is 2,7-diazaspiro[3.5]nonanyl and C is piperidyl.

In one embodiment, the present invention provides a compound of formula (I) as described herein, or a pharmaceutically acceptable salt thereof, wherein B is 6-azaspiro[3.4]octanyl and C is pyrrolidinyl.

In one embodiment, the present invention provides a compound of formula (I) as described herein, or a pharmaceutically acceptable salt thereof, wherein B is cubanyl and C is pyrrolidinyl.

In one embodiment, the present invention provides a compound of formula (I) as described herein, or a pharmaceutically acceptable salt thereof, wherein B is piperazinyl and C is pyrrolidinyl.

In one embodiment, the present invention provides a compound of formula (I) as described herein, or a pharmaceutically acceptable salt thereof, wherein B is bicyclo[1.1.1]pentanyl and C is pyrrolidinyl.

In one embodiment, the present invention provides a compound of formula (I) as described herein, or a pharmaceutically acceptable salt thereof, wherein B is 8-azabicyclo[3.2.1]octanyl and C is pyrrolidinyl.

In one embodiment, the present invention provides a compound of formula (I) as described herein, or a pharmaceutically acceptable salt thereof, wherein B is 1,2,3,3a,4,5,6,6a-octahydrocyclopenta[c]pyrrolyl and C is pyrrolidinyl.

In one embodiment, the present invention provides a compound of formula (I) as described herein, or a pharmaceutically acceptable salt thereof, wherein B is cyclobutyl and C is pyrrolidinyl.

In one embodiment, the present invention provides a compound of formula (I) as described herein, or a pharmaceutically acceptable salt thereof, wherein B is bicyclo[1.1.1]pentanyl and C is pyrrolidinyl.

In one embodiment, the present invention provides a compound of formula (I) as described herein, or a pharmaceutically acceptable salt thereof, wherein B is spiro[3.3]heptanyl and C is pyrrolidinyl.

In one embodiment, the present invention provides a compound of formula (I) as described herein, or a pharmaceutically acceptable salt thereof, wherein L¹ is selected from a covalent bond, carbonyl, —CH₂—, —CH₂O—, —NHCH₂—, CH₂NH—, —NR¹¹—, —C(O)—NH—NH—C(O)—CH₂—, —SO₂—, and —O—.

In a preferred embodiment, the present invention provides a compound of formula (I) as described herein, or a pharmaceutically acceptable salt thereof, wherein R⁵ is a group

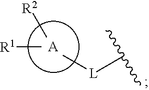

wherein R¹, R², A and L are as defined herein.

In a preferred embodiment, the present invention provides a compound of formula (I) as described herein, or a pharmaceutically acceptable salt thereof, wherein R¹ is selected from halogen, C₁₋₆-alkyl, halo-C₁₋₆-alkyl, and a group

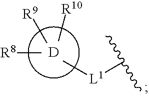

wherein R⁸ to R¹⁰, D, and L¹ are as defined herein.

In a preferred embodiment, the present invention provides a compound of formula (I) as described herein, or a pharmaceutically acceptable salt thereof, wherein R² is selected from hydrogen, halogen, and halo-C₁₋₆-alkyl.

In a preferred embodiment, the present invention provides a compound of formula (I) as described herein, or a pharmaceutically acceptable salt thereof, wherein R^{2a} is hydrogen.

In a preferred embodiment, the present invention provides a compound of formula (I) as described herein, or a pharmaceutically acceptable salt thereof, wherein R³ is hydrogen.

In a preferred embodiment, the present invention provides a compound of formula (I) as described herein, or a pharmaceutically acceptable salt thereof, wherein R⁴ is selected from a group

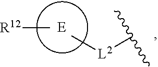

and carbamoyl.

In a preferred embodiment, the present invention provides a compound of formula (I) as described herein, or a pharmaceutically acceptable salt thereof, wherein R^{4a} is hydrogen.

In a preferred embodiment, the present invention provides a compound of formula (I) as described herein, or a pharmaceutically acceptable salt thereof, wherein R⁸ and R⁹ are each independently selected from halogen and halo-C₁₋₆-alkyl.

In a preferred embodiment, the present invention provides a compound of formula (I) as described herein, or a pharmaceutically acceptable salt thereof, wherein R¹⁰ is hydrogen.

In a preferred embodiment, the present invention provides a compound of formula (I) as described herein, or a pharmaceutically acceptable salt thereof, wherein R¹² is selected from hydrogen and oxo.

In a preferred embodiment, the present invention provides a compound of formula (I) as described herein, or a pharmaceutically acceptable salt thereof, wherein A is selected from C₆₋₁₄-aryl and C₃₋₁₀-cycloalkyl.

In a preferred embodiment, the present invention provides a compound of formula (I) as described herein, or a pharmaceutically acceptable salt thereof, wherein B is selected from spiro[3.3]heptan-2-yl, azetidinyl, and 7-azaspiro[3.5]nonan-7-yl.

In a preferred embodiment, the present invention provides a compound of formula (I) as described herein, or a pharmaceutically acceptable salt thereof, wherein D is selected from C₆₋₁₄-aryl and C₃₋₁₀-cycloalkyl.

In a preferred embodiment, the present invention provides a compound of formula (I) as described herein, or a pharmaceutically acceptable salt thereof, wherein E is selected from 5-membered heteroaryl and 5-membered heterocyclyl.

In a preferred embodiment, the present invention provides a compound of formula (I) as described herein, or a pharmaceutically acceptable salt thereof, wherein L is selected from a covalent bond, —O— and —CH₂O—.

In a preferred embodiment, the present invention provides a compound of formula (I) as described herein, or a pharmaceutically acceptable salt thereof, wherein L¹ is a covalent bond.

In a preferred embodiment, the present invention provides a compound of formula (I) as described herein, or a pharmaceutically acceptable salt thereof, wherein L² is a covalent bond.

In a particularly preferred embodiment, the present invention provides a compound of formula (I) as described herein, or a pharmaceutically acceptable salt thereof, wherein R¹ is selected from fluoro, chloro, tert-butyl, CHF₂, CF₃, and a group

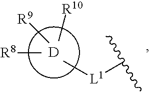

In a particularly preferred embodiment, the present invention provides a compound of formula (I) as described herein, or a pharmaceutically acceptable salt thereof, wherein R² is selected from hydrogen, fluoro, chloro, and CF₃.

In a particularly preferred embodiment, the present invention provides a compound of formula (I) as described herein, or a pharmaceutically acceptable salt thereof, wherein R⁸ and R⁹ are each independently selected from fluoro, chloro and CF₃.

In a particularly preferred embodiment, the present invention provides a compound of formula (I) as described herein, or a pharmaceutically acceptable salt thereof, wherein A is selected from phenyl and cyclopropyl.

In a particularly preferred embodiment, the present invention provides a compound of formula (I) as described herein, or a pharmaceutically acceptable salt thereof, wherein A is phenyl.

In a particularly preferred embodiment, the present invention provides a compound of formula (I) as described herein, or a pharmaceutically acceptable salt thereof, wherein A is cyclopropyl.

In a particularly preferred embodiment, the present invention provides a compound of formula (I) as described herein, or a pharmaceutically acceptable salt thereof, wherein D is selected from phenyl and cyclopropyl.

In a particularly preferred embodiment, the present invention provides a compound of formula (I) as described herein, or a pharmaceutically acceptable salt thereof, wherein D is phenyl.

In a particularly preferred embodiment, the present invention provides a compound of formula (I) as described herein, or a pharmaceutically acceptable salt thereof, wherein D is cyclopropyl.

In a particularly preferred embodiment, the present invention provides a compound of formula (I) as described herein, or a pharmaceutically acceptable salt thereof, wherein E is selected from imidazolyl, pyrazolyl, triazolyl, and oxazolidinyl.

In a particularly preferred embodiment, the present invention provides a compound of formula (I) as described herein, or a pharmaceutically acceptable salt thereof, wherein E is imidazolyl.

In a particularly preferred embodiment, the present invention provides a compound of formula (I) as described herein, or a pharmaceutically acceptable salt thereof, wherein E is pyrazolyl.

In a particularly preferred embodiment, the present invention provides a compound of formula (I) as described herein, or a pharmaceutically acceptable salt thereof, wherein E is triazolyl.

In a particularly preferred embodiment, the present invention provides a compound of formula (I) as described herein, or a pharmaceutically acceptable salt thereof, wherein E is oxazolidinyl.

In one embodiment, the present invention provides a compound of formula (I) as described herein, or a pharmaceutically acceptable salt thereof, selected from:
[3-[[2-Fluoro-6-(trifluoromethyl)phenyl]methoxy]azetidin-1-yl]-[3-(1H-pyrazol-5-yl)pyrrolidin-1-yl]methanone;
[3-[[2-Fluoro-4-(trifluoromethyl)phenyl]methoxy]azetidin-1-yl]-[3-(1H-pyrazol-5-yl)pyrrolidin-1-yl]methanone;
4-[1-[3-[[2-Fluoro-4-(trifluoromethyl)phenyl]methoxy]azetidine-1-carbonyl]pyrrolidin-3-yl]oxazolidin-2-one;
(+)- or (−)-[3-[[2-Fluoro-4-(trifluoromethyl)phenyl]methoxy]azetidin-1-yl]-[3-(1H-pyrazol-5-yl)pyrrolidin-1-yl]methanone;
(−)- or (+)-[3-[[2-Fluoro-4-(trifluoromethyl)phenyl]methoxy]azetidin-1-yl]-[3-(1H-pyrazol-5-yl)pyrrolidin-1-yl]methanone;
[3-[[2-Fluoro-4-(trifluoromethyl)phenyl]methoxy]azetidin-1-yl]-[3-(4-methyl-1H-pyrazol-5-yl)azetidin-1-yl]methanone;
[3-[[2-Fluoro-4-(trifluoromethyl)phenyl]methoxy]azetidin-1-yl]-[3-(1H-pyrazol-5-yl)azetidin-1-yl]methanone;
[4-[(2-Chloro-4-fluoro-phenyl)methoxy]-1-piperidyl]-[3-(4-methyl-1H-pyrazol-5-yl)azetidin-1-yl]methanone;
[4-[(4-Chloro-2-fluoro-phenoxy)methyl]-1-piperidyl]-[3-(1H-pyrazol-5-yl)pyrrolidin-1-yl]methanone;
[3-[[2-Fluoro-4-(trifluoromethyl)phenyl]methoxy]azetidin-1-yl]-[3-(1H-pyrazol-5-yl)-1-piperidyl]methanone;
[6-(2-Chloro-4-fluoro-phenoxy)-2-azaspiro[3.3]heptan-2-yl]-[3-(1H-pyrazol-5-yl)pyrrolidin-1-yl]methanone;
[3-(1H-Pyrazol-5-yl)pyrrolidin-1-yl]-[4-[[4-(trifluoromethyl)phenyl]methyl]-1-piperidyl]methanone;
[3-[[2-Fluoro-4-(trifluoromethyl)phenyl]methoxy]azetidin-1-yl]-[3-(2-methylpyrazol-3-yl)pyrrolidin-1-yl]methanone;
[3-[[2-Fluoro-4-(trifluoromethyl)phenyl]methoxy]azetidin-1-yl]-[3-(1H-pyrazol-5-yl)phenyl]methanone;
[3-[[2-Fluoro-4-(pentafluoro-26-sulfanyl)phenyl]methoxy]azetidin-1-yl]-[3-(1H-pyrazol-5-yl)pyrrolidin-1-yl]methanone;
(+)- or (−)-[3-[[2-Fluoro-6-(trifluoromethyl)phenyl]methoxy]azetidin-1-yl]-[3-(1H-pyrazol-5-yl)pyrrolidin-1-yl]methanone;
(−)- or (+)-[3-[[2-Fluoro-6-(trifluoromethyl)phenyl]methoxy]azetidin-1-yl]-[3-(1H-pyrazol-5-yl)pyrrolidin-1-yl]methanone;
[3-[4-(2,4-Dichlorophenyl)phenyl]azetidin-1-yl]-[3-(1H-pyrazol-5-yl)pyrrolidin-1-yl]methanone;
[3-[[2-Fluoro-4-(trifluoromethyl)phenyl]methoxy]azetidin-1-yl]-[3-(1H-1,2,4-triazol-5-yl)pyrrolidin-1-yl]methanone;
6-[1-[3-[[2-Fluoro-4-(trifluoromethyl)phenyl]methoxy]azetidine-1-carbonyl]pyrrolidin-3-yl]-1H-pyridin-2-one;
6-[1-[3-[[2-Fluoro-4-(trifluoromethyl)phenyl]methoxy]azetidine-1-carbonyl]pyrrolidin-3-yl]-1H-pyrazin-2-one;
[3-[[2-Fluoro-4-(trifluoromethyl)phenyl]methoxy]azetidin-1-yl]-[3-(4-isopropyl-1H-pyrazol-3-yl)pyrrolidin-1-yl]methanone;
4-[1-[3-[[2-Fluoro-4-(trifluoromethyl)phenyl]methoxy]azetidine-1-carbonyl]pyrrolidin-3-yl]oxazolidin-2-one;
Ethyl 5-[1-[3-[[2-fluoro-4-(trifluoromethyl)phenyl]methoxy]azetidine-1-carbonyl]pyrrolidin-3-yl]-1H-pyrazole-4-carboxylate;
5-[1-[3-[[2-Fluoro-4-(trifluoromethyl)phenyl]methoxy]azetidine-1-carbonyl]pyrrolidin-3-yl]pyrrolidin-2-one;
[3-[[2-Fluoro-4-(trifluoromethyl)phenyl]methoxy]azetidin-1-yl]-[(3R)-3-(1H-1,2,4-triazol-5-yl)pyrrolidin-1-yl]methanone;
[3-[[2-Fluoro-4-(trifluoromethyl)phenyl]methoxy]azetidin-1-yl]-[(3S)-3-(1H-1,2,4-triazol-5-yl)pyrrolidin-1-yl]methanone;
1-[3-[[2-Fluoro-4-(trifluoromethyl)phenyl]methoxy]azetidine-1-carbonyl]pyrrolidine-3-carboxamide;
[3-[[2-Fluoro-4-(trifluoromethyl)phenyl]methoxy]azetidin-1-yl]-[3-(1H-triazol-5-yl)pyrrolidin-1-yl]methanone;
[3-(1H-1,2,4-Triazol-5-yl)pyrrolidin-1-yl]-[3-[4-[1-(trifluoromethyl)cyclopropyl]phenyl]azetidin-1-yl]methanone;
[3-(4-tert-Butylphenyl)azetidin-1-yl]-[3-(1H-1,2,4-triazol-5-yl)pyrrolidin-1-yl]methanone;
[3-[[2-Fluoro-4-(trifluoromethyl)phenyl]methoxy]azetidin-1-yl]-[3-(1,2,4-triazol-1-yl)pyrrolidin-1-yl]methanone;
[3-[[2-Fluoro-4-(trifluoromethyl)phenyl]methoxy]azetidin-1-yl]-(3-imidazol-1-ylpyrrolidin-1-yl)methanone;
[3-[[2,4-bis(Trifluoromethyl)phenyl]methoxy]azetidin-1-yl]-[3-(1H-1,2,4-triazol-5-yl)pyrrolidin-1-yl]methanone;
(+)- or (−)-[2-(2-Chloro-4-fluoro-phenoxy)-7-azaspiro[3.5]nonan-7-yl]-[(3R)-3-(1H-1,2,4-triazol-5-yl)pyrrolidin-1-yl]methanone;
(−)- or (+)-[2-(2-Chloro-4-fluoro-phenoxy)-7-azaspiro[3.5]nonan-7-yl]-[(3S)-3-(1H-1,2,4-triazol-5-yl)pyrrolidin-1-yl]methanone;
(+)- or (−)-[3-(1H-Pyrazol-5-yl)pyrrolidin-1-yl]-[3-(4-tetrahydropyran-4-ylphenyl)azetidin-1-yl]methanone;
(−)- or (+)-[3-(1H-Pyrazol-5-yl)pyrrolidin-1-yl]-[3-(4-tetrahydropyran-4-ylphenyl)azetidin-1-yl]methanone;
(+)- or (−)-[3-(4-Isopropoxyphenyl)azetidin-1-yl]-[3-(1H-pyrazol-5-yl)pyrrolidin-1-yl]methanone;
(−)- or (+)-[3-(4-Isopropoxyphenyl)azetidin-1-yl]-[(3S)-3-(1H-pyrazol-5-yl)pyrrolidin-1-yl]methanone;
(+)- or (−)-[6-[(2,4-Difluorophenyl)methyl]-2-azaspiro[3.3]heptan-2-yl]-[3-(1H-1,2,4-triazol-5-yl)pyrrolidin-1-yl]methanone;
(−)- or (+)-[6-[(2,4-Difluorophenyl)methyl]-2-azaspiro[3.3]heptan-2-yl]-[3-(1H-1,2,4-triazol-5-yl)pyrrolidin-1-yl]methanone;
[3-[[2-Fluoro-4-(trifluoromethyl)phenyl]methoxy]azetidin-1-yl]-[3-(1H-imidazol-5-yl)pyrrolidin-1-yl]methanone;
(+)- or (−)-[2-[2-Fluoro-4-(trifluoromethyl) phenoxy]-7-azaspiro[3.5]nonan-7-yl]-[3-(1H-1,2,4-triazol-5-yl)pyrrolidin-1-yl]methanone;
(−)- or (+)-[2-[2-Fluoro-4-(trifluoromethyl) phenoxy]-7-azaspiro[3.5]nonan-7-yl]-[3-(1H-1,2,4-triazol-5-yl)pyrrolidin-1-yl]methanone;
[3-[[2-Fluoro-4-(trifluoromethyl)phenyl]methoxy]azetidin-1-yl]-[3-(1H-imidazol-2-yl)pyrrolidin-1-yl]methanone;
[3-[[2-Fluoro-4-(trifluoromethyl)phenyl]methoxy]azetidin-1-yl]-[(3S)-3-(1H-triazol-5-yl)pyrrolidin-1-yl]methanone;
Sodium [3-[[2-fluoro-4-(trifluoromethyl)phenyl]methoxy]azetidin-1-yl]-[rac-(3S)-3-(1H-triazol-5-yl)pyrrolidin-1-yl]methanone;
[3-[[2-Fluoro-4-(trifluoromethyl)phenyl]methoxy]azetidin-1-yl]-[(3R)-3-(1H-triazol-5-yl)pyrrolidin-1-yl]methanone;
(+)- or (−)-[3-[[2-Fluoro-4-(trifluoromethyl)phenyl]methoxy]azetidin-1-yl]-[3-imidazol-1-ylpyrrolidin-1-yl]methanone;
(−)- or (+)-[3-[[2-Fluoro-4-(trifluoromethyl)phenyl]methoxy]azetidin-1-yl]-[3-imidazol-1-ylpyrrolidin-1-yl]methanone;
[3-[[3-Chloro-4-(trifluoromethyl)phenyl]methoxy]azetidin-1-yl]-[3-(1H-1,2,4-triazol-5-yl)pyrrolidin-1-yl]methanone;
[3-[[2-Chloro-4-(trifluoromethyl)phenyl]methoxy]azetidin-1-yl]-[3-(1H-1,2,4-triazol-5-yl)pyrrolidin-1-yl]methanone;
(+)- or (−)-[3-[2-[2-Fluoro-4-(trifluoromethyl)phenyl]ethyl]azetidin-1-yl]-[3-(1H-1,2,4-triazol-5-yl)pyrrolidin-1-yl]methanone;
(−)- or (+)-[3-[2-[2-Fluoro-4-(trifluoromethyl)phenyl]ethyl]azetidin-1-yl]-[3-(1H-1,2,4-triazol-5-yl)pyrrolidin-1-yl]methanone;
(+)- or (−)-[3-(4-Propoxyphenyl)azetidin-1-yl]-[3-(1H-pyrazol-5-yl)pyrrolidin-1-yl]methanone;
(−)- or (+)-[3-(4-Propoxyphenyl)azetidin-1-yl]-[3-(1H-pyrazol-5-yl)pyrrolidin-1-yl]methanone;
(+)- or (−)-[3-[[2,4-bis(Trifluoromethyl)phenyl]methoxy]azetidin-1-yl]-[3-(1H-1,2,4-triazol-5-yl)pyrrolidin-1-yl]methanone;
(−)- or (+)-[3-[[2,4-bis(Trifluoromethyl)phenyl]methoxy]azetidin-1-yl]-[3-(1H-1,2,4-triazol-5-yl)pyrrolidin-1-yl]methanone;
[3-[[2-Fluoro-4-(trifluoromethyl)phenyl]methoxy]azetidin-1-yl]-[3-(1H-tetrazol-5-yl)pyrrolidin-1-yl]methanone;
(+)- or (−)-1-[3-[[2-Fluoro-4-(trifluoromethyl)phenyl]methoxy]azetidine-1-carbonyl]-N-methyl-pyrrolidine-3-carboxamide;
(−)- or (+)-1-[3-[[2-Fluoro-4-(trifluoromethyl)phenyl]methoxy]azetidine-1-carbonyl]-N-methyl-pyrrolidine-3-carboxamide;
(+)- or (−)-1-[3-[[2-Fluoro-4-(trifluoromethyl)phenyl]methoxy]azetidine-1-carbonyl]-N,N-dimethyl-pyrrolidine-3-carboxamide;
(−)- or (+)-1-[3-[[2-Fluoro-4-(trifluoromethyl)phenyl]methoxy]azetidine-1-carbonyl]-N,N-dimethyl-pyrrolidine-3-carboxamide;
(+)- or (−)-[3-[[2-Fluoro-4-(trifluoromethyl)phenyl]methoxy]azetidin-1-yl]-[3-pyrazol-1-ylpyrrolidin-1-yl]methanone;
(−)- or (+)-[3-[[2-Fluoro-4-(trifluoromethyl)phenyl]methoxy]azetidin-1-yl]-[3-pyrazol-1-ylpyrrolidin-1-yl]methanone;
(+)- or (−)-1-[3-[4-(2,4-Difluorophenyl)phenyl]azetidine-1-carbonyl]pyrrolidine-3-carboxamide;
(−)- or (+)-1-[3-[4-(2,4-Difluorophenyl)phenyl]azetidine-1-carbonyl]pyrrolidine-3-carboxamide;
[3-(1H-Pyrazol-5-yl)pyrrolidin-1-yl]-[3-(4-THF-3-ylphenyl)azetidin-1-yl]methanone;
[3-(4-tert-Butylphenyl)azetidin-1-yl]-[3-(1H-pyrazol-5-yl)pyrrolidin-1-yl]methanone;
[3-[2-[2-fluoro-4-(trifluoromethyl)phenyl]ethyl]azetidin-1-yl]-[(3S)-3-(1H-pyrazol-5-yl)pyrrolidin-1-yl]methanone;
[3-[[2-Fluoro-4-(trifluoromethyl)phenyl]methoxy]azetidin-1-yl]-[3-(triazol-1-yl)pyrrolidin-1-yl]methanone;
[6-[(2,4-difluorophenyl)methyl]-2-azaspiro[3.3]heptan-2-yl]-[(3S)-3-(1H-pyrazol-5-yl)pyrrolidin-1-yl]methanone;
[3-(1H-Pyrazol-5-yl)pyrrolidin-1-yl]-[3-(4-tetrahydropyran-3-ylphenyl)azetidin-1-yl]methanone;
[3-[[2-Fluoro-4-(trifluoromethyl)phenyl]methoxy]azetidin-1-yl]-(3-pyrrol-1-ylpyrrolidin-1-yl)methanone;
trans-[3-[[2-Fluoro-4-(trifluoromethyl)phenyl]methoxy]cyclobutyl]-[3-(1H-triazol-5-yl)pyrrolidin-1-yl]methanone;
(+)- or (−)-[3-[4-[(1R,5S)-8-Oxa-3-azabicyclo[3.2.1]octan-3-yl]phenyl]azetidin-1-yl]-[3-(1H-pyrazol-5-yl)pyrrolidin-1-yl]methanone;
(−)- or (+)-[3-[4-[(1R,5S)-8-Oxa-3-azabicyclo[3.2.1]octan-3-yl]phenyl]azetidin-1-yl]-[3-(1H-pyrazol-5-yl)pyrrolidin-1-yl]methanone;
(−)- or (+)-[3-[4-(2,4-Difluorophenyl)phenyl]azetidin-1-yl]-[3-(1H-1,2,4-triazol-5-yl)pyrrolidin-1-yl]methanone;
(+)- or (−)-[3-[4-(2,4-Difluorophenyl)phenyl]azetidin-1-yl]-[3-(1H-1,2,4-triazol-5-yl)pyrrolidin-1-yl]methanone;
[3-[4-(2,4-Difluorophenyl)phenyl]azetidin-1-yl]-[(3S)-3-(1H-pyrazol-5-yl)pyrrolidin-1-yl]methanone;
[4-[bis(4-Fluorophenyl)methyl]-1-piperidyl]-[3-(1H-1,2,4-triazol-5-yl)pyrrolidin-1-yl]methanone;
4-[1-[3-[3-Chloro-4-(trifluoromethoxy)phenyl]azetidine-1-carbonyl]pyrrolidin-3-yl]oxazolidin-2-one;
[3-[4-(2-Methylazetidin-1-yl)phenyl]azetidin-1-yl]-[(3S or 3R)-3-(1H-pyrazol-5-yl)pyrrolidin-1-yl]methanone;
[3-[4-(2-Methylazetidin-1-yl)phenyl]azetidin-1-yl]-[(3R or 3S)-3-(1H-pyrazol-5-yl)pyrrolidin-1-yl]methanone;
[3-[4-(3-Fluoropyrrolidin-1-yl)phenyl]azetidin-1-yl]-[(3R or 3S)-3-(1H-pyrazol-5-yl)pyrrolidin-1-yl]methanone;
(+)- or (−)-[3-(1H-1,2,4-Triazol-5-yl)pyrrolidin-1-yl]-[3-[4-[1-(trifluoromethyl)cyclopropyl]phenyl]azetidin-1-yl]methanone;
(−)- or (+)-[3-(1H-1,2,4-Triazol-5-yl)pyrrolidin-1-yl]-[3-[4-[1-(trifluoromethyl)cyclopropyl]phenyl]azetidin-1-yl]methanone;
(+)- or (−)-[3-[[2-Chloro-4-(trifluoromethyl)phenyl]methoxy]azetidin-1-yl]-[3-(1H-1,2,4-triazol-5-yl)pyrrolidin-1-yl]methanone;
(−)- or (+)-[3-[[2-Chloro-4-(trifluoromethyl)phenyl]methoxy]azetidin-1-yl]-[3-(1H-1,2,4-triazol-5-yl)pyrrolidin-1-yl]methanone;
[3-[4-(3-Fluoropyrrolidin-1-yl)phenyl]azetidin-1-yl]-[(3S or 3R)-3-(1H-pyrazol-5-yl)pyrrolidin-1-yl]methanone;
4-[1-[3-[4-(2,2,2-Trifluoroethyl)phenyl]azetidine-1-carbonyl]pyrrolidin-3-yl]oxazolidin-2-one;
[3-[4-(Oxetan-3-yl)phenyl]azetidin-1-yl]-[3-(1H-pyrazol-5-yl)pyrrolidin-1-yl]methanone;
(+)- or (−)-[3-[4-(Oxetan-3-yl)phenyl]azetidin-1-yl]-[3-(1H-pyrazol-5-yl)pyrrolidin-1-yl]methanone;
(−)- or (+)-[3-[4-(Oxetan-3-yl)phenyl]azetidin-1-yl]-[3-(1H-pyrazol-5-yl)pyrrolidin-1-yl]methanone;
4-[1-[3-[4-(2,4-Difluorophenyl)phenyl]azetidine-1-carbonyl]pyrrolidin-3-yl]oxazolidin-2-one;
[3-(1H-Triazol-5-yl)pyrrolidin-1-yl]-[3-[4-[1-(trifluoromethyl)cyclopropyl]phenyl]azetidin-1-yl]methanone;
[4-[bis(4-Fluorophenyl)methyl]-1-piperidyl]-[3-(1H-triazol-5-yl)pyrrolidin-1-yl]methanone;
4-[1-[3-[4-[1-(Trifluoromethyl)cyclopropyl]phenyl]azetidine-1-carbonyl]pyrrolidin-3-yl]oxazolidin-2-one;
(+)- or (−)-[3-(1H-Triazol-5-yl)pyrrolidin-1-yl]-[3-[4-[1-(trifluoromethyl)cyclopropyl]phenyl]azetidin-1-yl]methanone;
(−)- or (+)-[3-(1H-Triazol-5-yl)pyrrolidin-1-yl]-[3-[4-[1-(trifluoromethyl)cyclopropyl]phenyl]azetidin-1-yl]methanone;
(+)- or (−)-[3-(3,3-Dimethyl-2H-benzofuran-6-yl)azetidin-1-yl]-[3-(1H-pyrazol-5-yl)pyrrolidin-1-yl]methanone;
(−)- or (+)-[3-(3,3-Dimethyl-2H-benzofuran-6-yl)azetidin-1-yl]-[3-(1H-pyrazol-5-yl)pyrrolidin-1-yl]methanone;
(+)- or (−)-[4-[bis(4-Fluorophenyl)methyl]-1-piperidyl]-[3-(1H-triazol-5-yl)pyrrolidin-1-yl]methanone;
(−)- or (+)-[4-[bis(4-Fluorophenyl)methyl]-1-piperidyl]-[3-(1H-triazol-5-yl)pyrrolidin-1-yl]methanone;
[3-[[2-Chloro-4-(trifluoromethyl)phenyl]methoxy]azetidin-1-yl]-[(3S)-3-(1H-triazol-5-yl)pyrrolidin-1-yl]methanone;
[(3S)-3-(1H-Triazol-5-yl)pyrrolidin-1-yl]-[3-[4-(2,2,2-trifluoro-1,1-dimethyl-ethoxy)phenyl]azetidin-1-yl]methanone;
[3-[[2-Fluoro-4-(pentafluoro-26-sulfanyl)phenyl]methoxy]azetidin-1-yl]-[(3S)-3-(1H-triazol-5-yl)pyrrolidin-1-yl]methanone;
[6-[(2,4-Difluorophenyl)methyl]-2-azaspiro[3.3]heptan-2-yl]-[(3S)-3-(1H-triazol-5-yl)pyrrolidin-1-yl]methanone;
(+)-[4-[(2-Chloro-4-fluoro-phenoxy)methyl]-1-piperidyl]-[(3R)-3-(1H-triazol-5-yl)pyrrolidin-1-yl]methanone;
(−)-[4-[(2-Chloro-4-fluoro-phenoxy)methyl]-1-piperidyl]-[(3S)-3-(1H-triazol-5-yl)pyrrolidin-1-yl]methanone;
(+)- or (−)-[3-(1H-1,2,4-Triazol-5-yl)pyrrolidin-1-yl]-[3-[4-(2,2,2-trifluoro-1,1-dimethyl-ethoxy)phenyl]azetidin-1-yl]methanone;
(−)- or (+)-[3-(1H-1,2,4-Triazol-5-yl)pyrrolidin-1-yl]-[3-[4-(2,2,2-trifluoro-1,1-dimethyl-ethoxy)phenyl]azetidin-1-yl]methanone;
(+)- or (−)-[4-[[2-Chloro-4-(trifluoromethyl)phenyl]methoxy]-1-piperidyl]-[(3R)-3-(1H-triazol-5-yl)pyrrolidin-1-yl]methanone;
(−)- or (+)-[4-[[2-Chloro-4-(trifluoromethyl)phenyl]methoxy]-1-piperidyl]-[(3S)-3-(1H-triazol-5-yl)pyrrolidin-1-yl]methanone;
[(3R or 3S)-3-(1H-Triazol-5-yl)pyrrolidin-1-yl]-[3-[[4-(trifluoromethyl)phenyl]methyl]pyrrolidin-1-yl]methanone;
[3-[[2-Fluoro-4-(trifluoromethyl)phenyl]methoxy]-8-azabicyclo[3.2.1]octan-8-yl]-[(3R or 3S)-3-(1H-triazol-5-yl)pyrrolidin-1-yl]methanone;
[3-[[2-Fluoro-4-(trifluoromethyl)phenyl]methoxy]-8-azabicyclo[3.2.1]octan-8-yl]-[(3S or 3R)-3-(1H-triazol-5-yl)pyrrolidin-1-yl]methanone;
[(3aS,6aR)-5-[2-Fluoro-4-(trifluoromethyl) phenoxy]-3,3a,4,5,6,6a-hexahydro-1H-cyclopenta[c]pyrrol-2-yl]-[(3R or 3S)-3-(1H-triazol-5-yl)pyrrolidin-1-yl]methanone;
[(3aS,6aR)-5-[2-Fluoro-4-(trifluoromethyl) phenoxy]-3,3a,4,5,6,6a-hexahydro-1H-cyclopenta[c]pyrrol-2-yl]-[(3S or 3R)-3-(1H-triazol-5-yl)pyrrolidin-1-yl]methanone;
(−)-[3-[[2-Fluoro-4-(trifluoromethyl)phenyl]methoxy]-2-methyl-azetidin-1-yl]-[(3R or 3S)-3-(1H-triazol-5-yl)pyrrolidin-1-yl]methanone;
(+)-[3-[[2-Fluoro-4-(trifluoromethyl)phenyl]methoxy]-2-methyl-azetidin-1-yl]-[(3R or 3S)-3-(1H-triazol-5-yl)pyrrolidin-1-yl]methanone;
(−)-[3-[[2-Fluoro-4-(trifluoromethyl)phenyl]methoxy]-2-methyl-azetidin-1-yl]-[(3R or 3S)-3-(1H-triazol-5-yl)pyrrolidin-1-yl]methanone;
(+)-[3-[[2-Fluoro-4-(trifluoromethyl)phenyl]methoxy]-2-methyl-azetidin-1-yl]-[(3S or 3R)-3-(1H-triazol-5-yl)pyrrolidin-1-yl]methanone;
(−)-[3-[[2-Fluoro-4-(trifluoromethyl)phenyl]methoxy]-2-methyl-azetidin-1-yl]-[(3S or 3R)-3-(1H-triazol-5-yl)pyrrolidin-1-yl]methanone;
(+)-[3-[[2-Fluoro-4-(trifluoromethyl)phenyl]methoxy]-2-methyl-azetidin-1-yl]-[(3S or 3R)-3-(1H-triazol-5-yl)pyrrolidin-1-yl]methanone;
[3-[6-(2-Chlorophenoxy)-3-pyridyl]azetidin-1-yl]-[3-(tetrazol-1-yl)pyrrolidin-1-yl]methanone;
[3-[[2-Fluoro-4-(trifluoromethyl)phenyl]methoxy]azetidin-1-yl]-[(3S)-3-(1,2,4-triazol-4-yl)pyrrolidin-1-yl]methanone;
[3-[[2-Fluoro-4-(trifluoromethyl)phenyl]methoxy]azetidin-1-yl]-[(3R)-3-(1,2,4-triazol-4-yl)pyrrolidin-1-yl]methanone;
[3-(4H-1,2,4-Triazol-3-yl)pyrrolidin-1-yl]-[3-[4-[3-(2,2,2-trifluoroethoxy)azetidin-1-yl]phenyl]azetidin-1-yl]methanone;
[6-(2-Chloro-4-fluoro-phenoxy)-2-azaspiro[3.3]heptan-2-yl]-[3-(4H-1,2,4-triazol-3-yl)pyrrolidin-1-yl]methanone;
[3-(4H-1,2,4-Triazol-3-yl)pyrrolidin-1-yl]-[3-[6-[4-(trifluoromethoxy) phenoxy]-3-pyridyl]azetidin-1-yl]methanone;
[(3S)-3-(1,2,4-Triazol-4-yl)pyrrolidin-1-yl]-[3-[5-[1-(trifluoromethyl)cyclopropyl]-1,2,4-oxadiazol-3-yl]azetidin-1-yl]methanone;
[(3S)-3-(1,2,4-Triazol-4-yl)pyrrolidin-1-yl]-[3-[4-[3-(2,2,2-trifluoroethoxy)azetidin-1-yl]phenyl]azetidin-1-yl]methanone;
[6-(2-Chloro-4-fluoro-phenoxy)-2-azaspiro[3.3]heptan-2-yl]-[(3S)-3-(1,2,4-triazol-4-yl)pyrrolidin-1-yl]methanone;
[(3R)-3-(1,2,4-Triazol-4-yl)pyrrolidin-1-yl]-[6-[6-(trifluoromethyl)pyrazin-2-yl]oxy-2-azaspiro[3.3]heptan-2-yl]methanone;
[(3R)-3-(1,2,4-Triazol-4-yl)pyrrolidin-1-yl]-[3-[6-[4-(trifluoromethoxy) phenoxy]-3-pyridyl]azetidin-1-yl]methanone;
[3-[4-(4-Fluorophenoxy)phenyl]azetidin-1-yl]-[3-(tetrazol-1-yl)pyrrolidin-1-yl]methanone;
[3-(Tetrazol-1-yl)pyrrolidin-1-yl]-[6-[6-(trifluoromethyl)pyrazin-2-yl]oxy-2-azaspiro[3.3]heptan-2-yl]methanone;
[3-(Tetrazol-1-yl)pyrrolidin-1-yl]-[3-[6-[4-(trifluoromethoxy) phenoxy]-3-pyridyl]azetidin-1-yl]methanone;
(+)- or (−)-[3-(4H-1,2,4-Triazol-3-yl)pyrrolidin-1-yl]-[3-[4-[3-(2,2,2-trifluoroethoxy)azetidin-1-yl]phenyl]azetidin-1-yl]methanone;
(−)- or (+)-[3-(4H-1,2,4-Triazol-3-yl)pyrrolidin-1-yl]-[3-[4-[3-(2,2,2-trifluoroethoxy)azetidin-1-yl]phenyl]azetidin-1-yl]methanone;
(+)- or (−)-[6-(2-Chloro-4-fluoro-phenoxy)-2-azaspiro[3.3]heptan-2-yl]-[3-(4H-1,2,4-triazol-3-yl)pyrrolidin-1-yl]methanone;
(−)- or (+)-[6-(2-Chloro-4-fluoro-phenoxy)-2-azaspiro[3.3]heptan-2-yl]-[3-(4H-1,2,4-triazol-3-yl)pyrrolidin-1-yl]methanone;
(+)- or (−)-[3-(4H-1,2,4-Triazol-3-yl)pyrrolidin-1-yl]-[3-[6-[4-(trifluoromethoxy) phenoxy]-3-pyridyl]azetidin-1-yl]methanone;
(−)- or (+)-[3-(4H-1,2,4-Triazol-3-yl)pyrrolidin-1-yl]-[3-[6-[4-(trifluoromethoxy) phenoxy]-3-pyridyl]azetidin-1-yl]methanone;
(+)- or (−)-[3-[4-(4-Fluorophenoxy)phenyl]azetidin-1-yl]-[3-(tetrazol-1-yl)pyrrolidin-1-yl]methanone;
(−)- or (+)-[3-[4-(4-Fluorophenoxy)phenyl]azetidin-1-yl]-[3-(tetrazol-1-yl)pyrrolidin-1-yl]methanone;
(+)- or (−)-[3-(Tetrazol-1-yl)pyrrolidin-1-yl]-[6-[6-(trifluoromethyl)pyrazin-2-yl]oxy-2-azaspiro[3.3]heptan-2-yl]methanone;
(−)- or (+)-[3-(Tetrazol-1-yl)pyrrolidin-1-yl]-[6-[6-(trifluoromethyl)pyrazin-2-yl]oxy-2-azaspiro[3.3]heptan-2-yl]methanone;
(+)- or (−)-[3-(Tetrazol-1-yl)pyrrolidin-1-yl]-[3-[6-[4-(trifluoromethoxy) phenoxy]-3-pyridyl]azetidin-1-yl]methanone;
(−)- or (+)-[3-(Tetrazol-1-yl)pyrrolidin-1-yl]-[3-[6-[4-(trifluoromethoxy) phenoxy]-3-pyridyl]azetidin-1-yl]methanone;
[(3R)-3-(1H-Triazol-5-yl)pyrrolidin-1-yl]-[2-(3,3,3-trifluoropropoxy)spiro[3.3]heptan-6-yl]methanone;
[(3R)-3-(1H-Triazol-5-yl)pyrrolidin-1-yl]-[3-[5-[1-(trifluoromethyl)cyclopropyl]-1,2,4-oxadiazol-3-yl]azetidin-1-yl]methanone;
[3-[4-(4-Fluorophenoxy)phenyl]azetidin-1-yl]-[(3R)-3-(1H-triazol-5-yl)pyrrolidin-1-yl]methanone;
[(3R)-3-(1H-Triazol-5-yl)pyrrolidin-1-yl]-[3-[4-[3-(2,2,2-trifluoroethoxy)azetidin-1-yl]phenyl]azetidin-1-yl]methanone;
[6-(2-Chloro-4-fluoro-phenoxy)-2-azaspiro[3.3]heptan-2-yl]-[(3R)-3-(1H-triazol-5-yl)pyrrolidin-1-yl]methanone;
[(3R)-3-(1H-Triazol-5-yl)pyrrolidin-1-yl]-[3-[6-[4-(trifluoromethoxy) phenoxy]-3-pyridyl]azetidin-1-yl]methanone;
[(3R)-3-(1H-Triazol-5-yl)pyrrolidin-1-yl]-[3-[6-[3-(trifluoromethyl)pyrrolidin-1-yl]-3-pyridyl]azetidin-1-yl]methanone;
[3-[2-(3-Chlorophenyl) ethynyl]azetidin-1-yl]-[(3R)-3-(1H-triazol-5-yl)pyrrolidin-1-yl]methanone;
[3-[2-[2-(Difluoromethyl)phenyl]ethynyl]azetidin-1-yl]-[(3R)-3-(1H-triazol-5-yl)pyrrolidin-1-yl]methanone;
[4-(3-Cyclopropyl-1,2,4-oxadiazol-5-yl) piperazin-1-yl]-[rac-(3S)-3-(1H-triazol-5-yl)pyrrolidin-1-yl]methanone;
[(3S)-3-[3-Chloro-5-(2,2,2-trifluoroethoxy) phenoxy]pyrrolidin-1-yl]-[(3S or 3R)-3-(1H-triazol-5-yl)pyrrolidin-1-yl]methanone;
[(3S)-3-[3-chloro-5-(2,2,2-trifluoroethoxy) phenoxy]pyrrolidin-1-yl]-[(3R or 3S)-3-(1H-triazol-5-yl)pyrrolidin-1-yl]methanone;
[(3R)-3-[3-Chloro-5-(2,2,2-trifluoroethoxy) phenoxy]pyrrolidin-1-yl]-[(3S or 3R)-3-(1H-triazol-5-yl)pyrrolidin-1-yl]methanone;
[(3R)-3-[3-Chloro-5-(2,2,2-trifluoroethoxy) phenoxy]pyrrolidin-1-yl]-[(3R or 3S)-3-(1H-triazol-5-yl)pyrrolidin-1-yl]methanone;
[(3S)-3-(1H-Triazol-5-yl)pyrrolidin-1-yl]-[2-(3,3,3-trifluoropropoxy)spiro[3.3]heptan-6-yl]methanone;
[2-[[1-(Difluoromethyl)cyclopropyl]methoxy]spiro[3.3]heptan-6-yl]-[(3S)-3-(1H-triazol-5-yl)pyrrolidin-1-yl]methanone;
[8-(3-Cyclopropyl-1,2,4-oxadiazol-5-yl)-8-azabicyclo[3.2.1]octan-3-yl]-[(3S)-3-(1H-triazol-5-yl)pyrrolidin-1-yl]methanone;
[3-[4-(4-Chloro-2-methylsulfonyl-phenyl)phenyl]azetidin-1-yl]-[3-(triazol-1-yl)pyrrolidin-1-yl]methanone;
[3-[4-[3-(2,2-Dimethylpropyl)triazol-4-yl]phenyl]azetidin-1-yl]-[3-(triazol-1-yl)pyrrolidin-1-yl]methanone;
[3-(Triazol-1-yl)pyrrolidin-1-yl]-[3-[4-[1-(trifluoromethyl)cyclopropyl]phenyl]azetidin-1-yl]methanone;
[3-(2-Chloro-4-isopropylsulfonyl-phenyl)azetidin-1-yl]-[(3S)-3-(1H-triazol-5-yl)pyrrolidin-1-yl]methanone;
[3-(4-tert-Butylsulfonyl-2-chloro-phenyl)azetidin-1-yl]-[(3S)-3-(1H-triazol-5-yl)pyrrolidin-1-yl]methanone;
[3-[5-(2-Chlorophenoxy) pyrazin-2-yl]azetidin-1-yl]-[(3S)-3-(1H-triazol-5-yl)pyrrolidin-1-yl]methanone;
[(3S)-3-(1H-Triazol-5-yl)pyrrolidin-1-yl]-[6-[6-(trifluoromethyl)pyrazin-2-yl]oxy-2-azaspiro[3.3]heptan-2-yl]methanone;
[3-[4-(4-Chloro-2-methylsulfonyl-phenyl)phenyl]azetidin-1-yl]-[(3S)-3-(1H-triazol-5-yl)pyrrolidin-1-yl]methanone;
[(3S)-3-(1H-Triazol-5-yl)pyrrolidin-1-yl]-[3-[5-[1-(trifluoromethyl)cyclopropyl]-1,2,4-oxadiazol-3-yl]azetidin-1-yl]methanone;
[3-[6-(2,4-Dichlorophenoxy)-3-pyridyl]azetidin-1-yl]-[(3S)-3-(1H-triazol-5-yl)pyrrolidin-1-yl]methanone;
[3-[4-(4-Fluorophenoxy)phenyl]azetidin-1-yl]-[(3S)-3-(1H-triazol-5-yl)pyrrolidin-1-yl]methanone;
[3-(2-Chloro-4-isobutylsulfonyl-phenyl)azetidin-1-yl]-[(3S)-3-(1H-triazol-5-yl)pyrrolidin-1-yl]methanone;
[3-[4-(Benzenesulfonyl)phenyl]azetidin-1-yl]-[(3S)-3-(1H-triazol-5-yl)pyrrolidin-1-yl]methanone;
[3-(2-Chloro-4-propylsulfonyl-phenyl)azetidin-1-yl]-[(3S)-3-(1H-triazol-5-yl)pyrrolidin-1-yl]methanone;
[3-[4-(2-Chloro-4-methylsulfonyl-phenyl)phenyl]azetidin-1-yl]-[(3S)-3-(1H-triazol-5-yl)pyrrolidin-1-yl]methanone;
[3-(4-tert-Butylphenyl)azetidin-1-yl]-[(3S)-3-(1H-triazol-5-yl)pyrrolidin-1-yl]methanone;
1-[3-[4-(2-Chloro-4-methylsulfonyl-phenyl)phenyl]azetidine-1-carbonyl]pyrrolidine-3-carboxamide;
[3-(5-tert-Butyl-2-pyridyl)azetidin-1-yl]-[(3S)-3-(1H-triazol-5-yl)pyrrolidin-1-yl]methanone;
1-[3-[4-(2-Chloro-4-methylsulfonyl-phenyl)phenyl]azetidine-1-carbonyl]pyrrolidine-3-carboxylic acid;
1-[3-[4-(2,4-Difluorophenyl)phenyl]azetidine-1-carbonyl]pyrrolidine-3-carboxylic acid;
2-[4-[1-[(3S)-3-(1H-Triazol-5-yl)pyrrolidine-1-carbonyl]azetidin-3-yl]phenyl]sulfonylacetonitrile;
[3-[4-(N-Methylanilino)phenyl]azetidin-1-yl]-[(3S)-3-(1H-triazol-5-yl)pyrrolidin-1-yl]methanone;
[(3S)-3-(1H-Triazol-5-yl)pyrrolidin-1-yl]-[3-[4-(trifluoromethylsulfonyl)phenyl]azetidin-1-yl]methanone;
[3-(4-Cyclohexylsulfonylphenyl)azetidin-1-yl]-[(3S)-3-(1H-triazol-5-yl)pyrrolidin-1-yl]methanone;
1-[5-[1-[(3S)-3-(1H-Triazol-5-yl)pyrrolidine-1-carbonyl]azetidin-3-yl]-2-pyridyl]cyclobutanecarbonitrile;
[6-[(2,4-Difluorophenyl)methyl]-2,6-diazaspiro[3.3]heptan-2-yl]-[(3S)-3-(1H-triazol-5-yl)pyrrolidin-1-yl]methanone;
[6-[[4-Fluoro-2-(trifluoromethyl)phenyl]methyl]-2,6-diazaspiro[3.3]heptan-2-yl]-[(3S)-3-(1H-triazol-5-yl)pyrrolidin-1-yl]methanone;
[3-[4-[N-(Cyclopropylmethyl) anilino]phenyl]azetidin-1-yl]-[(3S)-3-(1H-triazol-5-yl)pyrrolidin-1-yl]methanone;
1-[3-[4-(4-Chloro-2-methylsulfonyl-phenyl)phenyl]azetidine-1-carbonyl]pyrrolidine-3-carboxamide;
[3-[4-(N-Phenylanilino)phenyl]azetidin-1-yl]-[(3S)-3-(1H-triazol-5-yl)pyrrolidin-1-yl]methanone;
[3-(6-tert-Butyl-3-pyridyl)azetidin-1-yl]-[(3S)-3-(1H-triazol-5-yl)pyrrolidin-1-yl]methanone;
[3-[6-[(5-Methoxy-2-pyridyl)-methyl-amino]-3-pyridyl]azetidin-1-yl]-[(3S)-3-(1H-triazol-5-yl)pyrrolidin-1-yl]methanone;
[3-[6-[(5-Ethyl-2-pyridyl)-methyl-amino]-3-pyridyl]azetidin-1-yl]-[(3S)-3-(1H-triazol-5-yl)pyrrolidin-1-yl]methanone;
[3-[6-[(5-Cyclopropyl-2-pyridyl)-methyl-amino]-3-pyridyl]azetidin-1-yl]-[(3S)-3-(1H-triazol-5-yl)pyrrolidin-1-yl]methanone;
[3-[4-(N-Methylanilino)phenyl]azetidin-1-yl]-[3-(1H-1,2,4-triazol-5-yl)pyrrolidin-1-yl]methanone;
[3-[6-(N-Methylanilino)-3-pyridyl]azetidin-1-yl]-[(3S)-3-(1H-triazol-5-yl)pyrrolidin-1-yl]methanone;
[3-[6-(4-Isopropyl-N-methyl-anilino)-3-pyridyl]azetidin-1-yl]-[(3S)-3-(1H-triazol-5-yl)pyrrolidin-1-yl]methanone;
[3-[6-[(3-Ethyl-2-pyridyl)-methyl-amino]-3-pyridyl]azetidin-1-yl]-[(3S)-3-(1H-triazol-5-yl)pyrrolidin-1-yl]methanone;
[3-[6-[N-(Cyclopropylmethyl) anilino]-3-pyridyl]azetidin-1-yl]-[(3S)-3-(1H-triazol-5-yl)pyrrolidin-1-yl]methanone;
(3S)-1-[3-[4-(4-Chloro-2-methylsulfonyl-phenyl)phenyl]azetidine-1-carbonyl]pyrrolidine-3-carboxamide;
(3R)-1-[3-[4-(4-Chloro-2-methylsulfonyl-phenyl)phenyl]azetidine-1-carbonyl]pyrrolidine-3-carboxamide;
[3-[6-[N-(Cyclopropylmethyl) anilino]-3-pyridyl]azetidin-1-yl]-[3-(1H-tetrazol-5-yl)pyrrolidin-1-yl]methanone;
[3-(1H-Tetrazol-5-yl)pyrrolidin-1-yl]-[3-[4-(2,2,2-trifluoro-1,1-dimethyl-ethoxy)phenyl]azetidin-1-yl]methanone;
[2-(2-Methoxyphenyl) sulfonyl-2,6-diazaspiro[3.3]heptan-6-yl]-[(3S)-3-(1H-triazol-5-yl)pyrrolidin-1-yl]methanone;
[2-[4-Fluoro-2-(trifluoromethyl)phenyl]sulfonyl-2,6-diazaspiro[3.3]heptan-6-yl]-[(3S)-3-(1H-triazol-5-yl)pyrrolidin-1-yl]methanone;
[2-(2,2-Dimethylpropylsulfonyl)-2,6-diazaspiro[3.3]heptan-6-yl]-[(3S)-3-(1H-triazol-5-yl)pyrrolidin-1-yl]methanone;
[3-[[2-Fluoro-4-(trifluoromethyl)phenyl]methoxy]azetidin-1-yl]-[(3S)-3-(4H-1,2,4-triazol-3-yl)pyrrolidin-1-yl]methanone;
[2-[4-Fluoro-2-(trifluoromethyl)phenyl]sulfonyl-2,6-diazaspiro[3.3]heptan-6-yl]-[(3R)-3-(4H-1,2,4-triazol-3-yl)pyrrolidin-1-yl]methanone;
[2-[4-Fluoro-2-(trifluoromethyl)phenyl]sulfonyl-2,6-diazaspiro[3.3]heptan-6-yl]-[(3S)-3-(4H-1,2,4-triazol-3-yl)pyrrolidin-1-yl]methanone;
[3-[6-[N-(Cyclopropylmethyl) anilino]-3-pyridyl]azetidin-1-yl]-[(3S)-3-(1H-tetrazol-5-yl)pyrrolidin-1-yl]methanone;
[3-[6-[N-(Cyclopropylmethyl) anilino]-3-pyridyl]azetidin-1-yl]-[(3R)-3-(1H-tetrazol-5-yl)pyrrolidin-1-yl]methanone;
[3-[4-(4-Fluorophenoxy)phenyl]azetidin-1-yl]-[3-(1H-tetrazol-5-yl)pyrrolidin-1-yl]methanone;
1-[3-[6-[N-(Cyclopropylmethyl) anilino]-3-pyridyl]azetidine-1-carbonyl]pyrrolidine-3-carboxamide;
[3-[4-(4-Fluorophenoxy)phenyl]azetidin-1-yl]-[(3R)-3-(1H-tetrazol-5-yl)pyrrolidin-1-yl]methanone;
[3-[4-(4-Fluorophenoxy)phenyl]azetidin-1-yl]-[(3S)-3-(1H-tetrazol-5-yl)pyrrolidin-1-yl]methanone;
[(3S)-3-(1,2,4-Triazol-4-yl)pyrrolidin-1-yl]-[3-[4-[4-(trifluoromethyl)pyrimidin-2-yl]oxyphenyl]azetidin-1-yl]methanone;
[3-[4-(4-Chloro-2-methylsulfonyl-phenyl)phenyl]azetidin-1-yl]-[3-(1H-tetrazol-5-yl)pyrrolidin-1-yl]methanone;
[(3S)-3-(1,2,4-Triazol-4-yl)pyrrolidin-1-yl]-[6-[6-(trifluoromethyl)pyrazin-2-yl]oxy-2-azaspiro[3.3]heptan-2-yl]methanone;
[(3S)-3-(1,2,4-Triazol-4-yl)pyrrolidin-1-yl]-[6-[[1-(trifluoromethyl)cyclopropyl]methoxy]-2-azaspiro[3.3]heptan-2-yl]methanone;
(3S)-1-[3-[6-[N-(Cyclopropylmethyl) anilino]-3-pyridyl]azetidine-1-carbonyl]pyrrolidine-3-carboxamide;
(3R)-1-[3-[6-[N-(Cyclopropylmethyl) anilino]-3-pyridyl]azetidine-1-carbonyl]pyrrolidine-3-carboxamide;
[6-[[4-Fluoro-2-(trifluoromethyl)phenyl]methyl]-2,6-diazaspiro[3.3]heptan-2-yl]-[(3S)-3-(4H-1,2,4-triazol-3-yl)pyrrolidin-1-yl]methanone;
[(3S)-3-(4H-1,2,4-Triazol-3-yl)pyrrolidin-1-yl]-[3-[4-[3-(trifluoromethyl)azetidin-1-yl]phenyl]azetidin-1-yl]methanone;
1-[3-[4-(Benzenesulfonyl)phenyl]azetidine-1-carbonyl]pyrrolidine-3-carboxamide;
[2-[[2-Fluoro-4-(trifluoromethyl)phenyl]methyl]-2,7-diazaspiro[3.5]nonan-7-yl]-[3-(4H-1,2,4-triazol-3-yl)pyrrolidin-1-yl]methanone;
1-[3-[4-[3-(2,2,2-Trifluoroethoxy)azetidin-1-yl]phenyl]azetidine-1-carbonyl]pyrrolidine-3-carboxamide;
[2-[(2-Chloro-4-fluoro-phenyl)methyl]-2,7-diazaspiro[3.5]nonan-7-yl]-[3-(4H-1,2,4-triazol-3-yl)pyrrolidin-1-yl]methanone;
[3-[(2-Fluoro-4-methylsulfonyl-phenyl)methoxy]azetidin-1-yl]-[3-(4H-1,2,4-triazol-3-yl)pyrrolidin-1-yl]methanone;
1-[3-[4-[1-(Trifluoromethyl)cyclopropyl]phenyl]azetidine-1-carbonyl]pyrrolidine-3-carboxamide;
1-[3-[4-[3-(Trifluoromethyl)azetidin-1-yl]phenyl]azetidine-1-carbonyl]pyrrolidine-3-carboxamide;
[3-[4-(4-Chloro-2-methylsulfonyl-phenyl)phenyl]azetidin-1-yl]-[(3R)-3-(1H-tetrazol-5-yl)pyrrolidin-1-yl]methanone;
[3-[4-(4-Chloro-2-methylsulfonyl-phenyl)phenyl]azetidin-1-yl]-[(3S)-3-(1H-tetrazol-5-yl)pyrrolidin-1-yl]methanone;
[3-[4-(4-Chloro-2-methylsulfonyl-phenyl)phenyl]azetidin-1-yl]-[(3S)-3-(1,2,4-triazol-4-yl)pyrrolidin-1-yl]methanone;
[6-[[2-Fluoro-4-(trifluoromethyl)phenyl]methyl]-2,6-diazaspiro[3.3]heptan-2-yl]-[3-(4H-1,2,4-triazol-3-yl)pyrrolidin-1-yl]methanone;
[3-(4H-1,2,4-Triazol-3-yl)pyrrolidin-1-yl]-[[3-[6-(trifluoromethyl)-3-pyridyl]methoxy]azetidin-1-yl]methanone;
[3-(4H-1,2,4-Triazol-3-yl)pyrrolidin-1-yl]-[3-[[5-(trifluoromethyl)-3-pyridyl]methoxy]azetidin-1-yl]methanone;
[3-(4H-1,2,4-Triazol-3-yl)pyrrolidin-1-yl]-[6-[[5-(trifluoromethyl)-3-pyridyl]oxy]-2-azaspiro[3.3]heptan-2-yl]methanone;
[6-[(5-Chloro-3-pyridyl)oxy]-2-azaspiro[3.3]heptan-2-yl]-[3-(4H-1,2,4-triazol-3-yl)pyrrolidin-1-yl]methanone;
[6-[(6-Chloro-3-pyridyl)oxy]-2-azaspiro[3.3]heptan-2-yl]-[3-(4H-1,2,4-triazol-3-yl)pyrrolidin-1-yl]methanone;
(3S)-1-[3-[4-[3-(2,2,2-Trifluoroethoxy)azetidin-1-yl]phenyl]azetidine-1-carbonyl]pyrrolidine-3-carboxamide;
(3R)-1-[3-[4-[3-(2,2,2-Trifluoroethoxy)azetidin-1-yl]phenyl]azetidine-1-carbonyl]pyrrolidine-3-carboxamide;
(3S)-1-[3-[4-(Benzenesulfonyl)phenyl]azetidine-1-carbonyl]pyrrolidine-3-carboxamide;
(3R)-1-[3-[4-(Benzenesulfonyl)phenyl]azetidine-1-carbonyl]pyrrolidine-3-carboxamide;
[3-[(2-Fluoro-4-methylsulfonyl-phenyl)methoxy]azetidin-1-yl]-[(3S)-3-(1H-triazol-5-yl)pyrrolidin-1-yl]methanone;
2-Cyclopropyl-6-[1-[(3S)-3-(1,2,4-triazol-4-yl)pyrrolidine-1-carbonyl]azetidin-3-yl]oxy-benzonitrile;
(3S)-1-[3-[[2-Fluoro-4-(trifluoromethyl)phenyl]methoxy]azetidine-1-carbonyl]pyrrolidine-3-carboxamide;
(3S)-1-[2-[2-(Trifluoromethyl)phenyl]sulfonyl-2,6-diazaspiro[3.3]heptane-6-carbonyl]pyrrolidine-3-carboxamide;
(3S)-1-[3-[[3-(Trifluoromethyl)phenyl]sulfonylamino]methyl]azetidine-1-carbonyl]pyrrolidine-3-carboxamide;
(3S)-1-[[3-[[4-(Trifluoromethyl)phenyl]sulfonylamino]methyl]azetidine-1-carbonyl]pyrrolidine-3-carboxamide;
(3S)-1-[6-[[1-(Trifluoromethyl)cyclopropyl]methoxy]-2-azaspiro[3.3]heptane-2-carbonyl]pyrrolidine-3-carboxamide;
(3S)-1-[6-[[6-(Trifluoromethyl)-3-pyridyl]oxy]-2-azaspiro[3.3]heptane-2-carbonyl]pyrrolidine-3-carboxamide;
(3S)-1-[6-[[5-(Trifluoromethyl)-2-pyridyl]methyl]-2,6-diazaspiro[3.3]heptane-2-carbonyl]pyrrolidine-3-carboxamide;
(3S)-1-[3-[[6-(Trifluoromethyl)-3-pyridyl]methoxy]azetidine-1-carbonyl]pyrrolidine-3-carboxamide;
(3S)-1-[[3-[5-(Trifluoromethyl)-3-pyridyl]methoxy]azetidine-1-carbonyl]pyrrolidine-3-carboxamide;
(3S)-1-[[6-[[1-(Trifluoromethyl)cyclopropyl]amino]methyl]-2-azaspiro[3.3]heptane-2-carbonyl]pyrrolidine-3-carboxamide;
[3-[4-(3-Chloropyridazin-4-yl)oxyphenyl]azetidin-1-yl]-[(3R)-3-(tetrazol-1-yl)pyrrolidin-1-yl]methanone;
[3-[[2-Fluoro-4-(trifluoromethyl)phenyl]methylamino]azetidin-1-yl]-[(3S)-3-(1H-triazol-5-yl)pyrrolidin-1-yl]methanone;
[3-[[2-Fluoro-4-(trifluoromethyl)phenyl]methylamino]azetidin-1-yl]-[3-(4H-1,2,4-triazol-3-yl)pyrrolidin-1-yl]methanone;
[3-[(2-Fluoro-4-methylsulfonyl-phenyl)methoxy]azetidin-1-yl]-[(3S)-3-(1,2,4-triazol-4-yl)pyrrolidin-1-yl]methanone;
[(3S)-3-(1,2,4-Triazol-4-yl)pyrrolidin-1-yl]-[2-[2-(trifluoromethyl)phenyl]sulfonyl-2,6-diazaspiro[3.3]heptan-6-yl]methanone;
1-[4-[1-[(3S)-3-(1,2,4-Triazol-4-yl)pyrrolidine-1-carbonyl]azetidin-3-yl]phenyl]cyclopropanecarbonitrile;
[3-[[2-Fluoro-4-(trifluoromethyl)phenyl]methylamino]azetidin-1-yl]-[(3S)-3-(1,2,4-triazol-4-yl)pyrrolidin-1-yl]methanone;
N-[2-[(3S)-3-(1,2,4-Triazol-4-yl)pyrrolidine-1-carbonyl]-2-azaspiro[3.3]heptan-6-yl]-1-(trifluoromethyl)cyclopropanecarboxamide;
[(3S)-3-(1,2,4-Triazol-4-yl)pyrrolidin-1-yl]-[3-[[4-[1-(trifluoromethyl)cyclopropyl]methylamino]phenyl]azetidin-1-yl]methanone;
(3S)-1-[3-[2-[1-(Trifluoromethyl)cyclopropyl]pyrimidin-5-yl]azetidine-1-carbonyl]pyrrolidine-3-carboxamide;
(3S)-1-[3-[6-[1-(Trifluoromethyl)cyclopropyl]-3-pyridyl]azetidine-1-carbonyl]pyrrolidine-3-carboxamide;
[3-(4H-1,2,4-Triazol-3-yl)pyrrolidin-1-yl]-[[3-[2-(trifluoromethyl)pyrimidin-5-yl]methoxy]azetidin-1-yl]methanone;
1-[3-[4-(4-Chloro-2-methylsulfonyl-phenyl)phenyl]azetidine-1-carbonyl]-N-methyl-pyrrolidine-3-carboxamide;
1-[3-[4-(4-Chloro-2-methylsulfonyl-phenyl)phenyl]azetidine-1-carbonyl]-N-ethyl-pyrrolidine-3-carboxamide;
(3S)-1-[[3-[4-[1-(Trifluoromethyl)cyclopropyl]amino]phenyl]azetidine-1-carbonyl]pyrrolidine-3-carboxamide;
(3S)-1-[3-[4-[[1-(Trifluoromethyl)cyclopropyl]methylamino]phenyl]azetidine-1-carbonyl]pyrrolidine-3-carboxamide;
(3S)-1-[3-[(2-Fluoro-4-methylsulfonyl-phenyl)methoxy]azetidine-1-carbonyl]pyrrolidine-3-carboxamide;
(3S)-1-[2-(2,4-Difluorophenyl) sulfonyl-2,6-diazaspiro[3.3]heptane-6-carbonyl]pyrrolidine-3-carboxamide;
(3S)-1-[2-[3-(Trifluoromethyl)phenyl]sulfonyl-2,6-diazaspiro[3.3]heptane-6-carbonyl]pyrrolidine-3-carboxamide;
(3S)-1-[2-[3-(Trifluoromethoxy)phenyl]sulfonyl-2,6-diazaspiro[3.3]heptane-6-carbonyl]pyrrolidine-3-carboxamide;
rac-(3S)-1-[3-[5-[3-(Trifluoromethyl)pyrrolidin-1-yl]-2-pyridyl]azetidine-1-carbonyl]pyrrolidine-3-carboxamide;
(3S)-1-[3-[4-(4-Chloro-2-methylsulfonyl-phenyl)phenyl]azetidine-1-carbonyl]-N-methyl-pyrrolidine-3-carboxamide;
(3R)-1-[3-[4-(4-Chloro-2-methylsulfonyl-phenyl)phenyl]azetidine-1-carbonyl]-N-methyl-pyrrolidine-3-carboxamide;
(3S)-1-[3-[4-(4-Chloro-2-methylsulfonyl-phenyl)phenyl]azetidine-1-carbonyl]-N-ethyl-pyrrolidine-3-carboxamide;
(3R)-1-[3-[4-(4-Chloro-2-methylsulfonyl-phenyl)phenyl]azetidine-1-carbonyl]-N-ethyl-pyrrolidine-3-carboxamide;
[3-[6-(2-Chlorophenoxy)-3-pyridyl]azetidin-1-yl]-[(3R)-3-(tetrazol-1-yl)pyrrolidin-1-yl]methanone;
[3-[4-(4-Fluorophenoxy)phenyl]azetidin-1-yl]-[(3S)-3-(tetrazol-1-yl)pyrrolidin-1-yl]methanone;
[3-[5-(2-Chlorophenoxy) pyrazin-2-yl]azetidin-1-yl]-[(3S)-3-(tetrazol-1-yl)pyrrolidin-1-yl]methanone;
[(3R)-3-(Tetrazol-1-yl)pyrrolidin-1-yl]-[6-[6-(trifluoromethyl)pyrazin-2-yl]oxy-2-azaspiro[3.3]heptan-2-yl]methanone;
2-Cyclopropyl-6-[1-[(3R)-3-(tetrazol-1-yl)pyrrolidine-1-carbonyl]azetidin-3-yl]oxy-benzonitrile;
[3-[5-(2-Chlorophenoxy) pyrazin-2-yl]azetidin-1-yl]-[(3R)-3-(tetrazol-1-yl)pyrrolidin-1-yl]methanone;
[(3S)-3-(Tetrazol-1-yl)pyrrolidin-1-yl]-[6-[6-(trifluoromethyl)pyrazin-2-yl]oxy-2-azaspiro[3.3]heptan-2-yl]methanone;
[3-[4-[3-(2,2-Dimethylpropyl)triazol-4-yl]phenyl]azetidin-1-yl]-[(3S)-3-(tetrazol-1-yl)pyrrolidin-1-yl]methanone;
[3-[4-(4-Fluorophenoxy)phenyl]azetidin-1-yl]-[(3R)-3-(tetrazol-1-yl)pyrrolidin-1-yl]methanone;
[3-[5-(2,4-Dichlorophenoxy) pyrazin-2-yl]azetidin-1-yl]-[(3R)-3-(tetrazol-1-yl)pyrrolidin-1-yl]methanone;
2-Cyclopropyl-6-[1-[(3S)-3-(tetrazol-1-yl)pyrrolidine-1-carbonyl]azetidin-3-yl]oxy-benzonitrile;
Methyl 2,2-dimethyl-3-[3-[1-[(3R)-3-(tetrazol-1-yl)pyrrolidine-1-carbonyl]azetidin-3-yl]oxyphenyl]propanoate;
[3-[6-(2-Chlorophenoxy)-3-pyridyl]azetidin-1-yl]-[(3S)-3-(tetrazol-1-yl)pyrrolidin-1-yl]methanone;
[3-[4-(4-Cyclopropylpyrimidin-2-yl)oxyphenyl]azetidin-1-yl]-[(3S)-3-(tetrazol-1-yl)pyrrolidin-1-yl]methanone;
[3-[5-(2,4-Dichlorophenoxy) pyrazin-2-yl]azetidin-1-yl]-[(3S)-3-(tetrazol-1-yl)pyrrolidin-1-yl]methanone;
1-[4-[1-[(3R)-3-(Tetrazol-1-yl)pyrrolidine-1-carbonyl]azetidin-3-yl]phenyl]cyclopropanecarbonitrile;
[3-[4-(4-Cyclopropylpyrimidin-2-yl)oxyphenyl]azetidin-1-yl]-[(3R)-3-(tetrazol-1-yl)pyrrolidin-1-yl]methanone;
1-[4-[1-[(3S)-3-(Tetrazol-1-yl)pyrrolidine-1-carbonyl]azetidin-3-yl]phenyl]cyclopropanecarbonitrile;
Methyl 2,2-dimethyl-3-[3-[1-[(3S)-3-(tetrazol-1-yl)pyrrolidine-1-carbonyl]azetidin-3-yl]oxyphenyl]propanoate;
[3-[4-[3-(2,2-Dimethylpropyl)triazol-4-yl]phenyl]azetidin-1-yl]-[(3R)-3-(tetrazol-1-yl)pyrrolidin-1-yl]methanone;
[3-[4-(4-Chloro-2-methylsulfonyl-phenyl)phenyl]azetidin-1-yl]-[(3R)-3-hydroxypyrrolidin-1-yl]methanone;
[3-[4-(4-Chloro-2-methylsulfonyl-phenyl)phenyl]azetidin-1-yl]-[(3R)-3-(hydroxymethyl)pyrrolidin-1-yl]methanone;
[3-[4-(4-Chloro-2-methylsulfonyl-phenyl)phenyl]azetidin-1-yl]-[(3S)-3-(hydroxymethyl)pyrrolidin-1-yl]methanone;
(3S)-1-[6-[[5-(Trifluoromethyl)pyrazin-2-yl]methyl]-2-azaspiro[3.3]heptane-2-carbonyl]pyrrolidine-3-carboxamide;
[7-[[2-Fluoro-4-(trifluoromethyl)phenyl]methyl]-2,7-diazaspiro[3.5]nonan-2-yl]-[3-(4H-1,2,4-triazol-3-yl)pyrrolidin-1-yl]methanone;
[7-[2-Fluoro-4-(trifluoromethyl) phenoxy]-2-azaspiro[3.5]nonan-2-yl]-[3-(4H-1,2,4-triazol-3-yl)pyrrolidin-1-yl]methanone;
[6-[2-Fluoro-4-(trifluoromethyl) anilino]-2-azaspiro[3.3]heptan-2-yl]-[3-(4H-1,2,4-triazol-3-yl)pyrrolidin-1-yl]methanone;
[6-[[2-Fluoro-4-(trifluoromethyl)phenyl]methylamino]-2-azaspiro[3.3]heptan-2-yl]-[3-(4H-1,2,4-triazol-3-yl)pyrrolidin-1-yl]methanone;
(3S)-1-[3-[4-[3-(Methylsulfonylmethyl)azetidin-1-yl]phenyl]azetidine-1-carbonyl]pyrrolidine-3-carboxamide;
rac-(3S)-1-[3-[5-[3-(Trifluoromethyl)pyrrolidin-1-yl]pyrazin-2-yl]azetidine-1-carbonyl]pyrrolidine-3-carboxamide;
[(3S)-3-(1,2,4-Triazol-4-yl)pyrrolidin-1-yl]-[3-[4-[3-(trifluoromethyl)azetidin-1-yl]phenyl]azetidin-1-yl]methanone;
[6-[[2-Fluoro-4-(trifluoromethyl)phenyl]methylamino]-2-azaspiro[3.3]heptan-2-yl]-[(3S)-3-(1,2,4-triazol-4-yl)pyrrolidin-1-yl]methanone;
[6-[(4,4-Difluoro-1-piperidyl)methyl]-2-azaspiro[3.3]heptan-2-yl]-[3-(4H-1,2,4-triazol-3-yl)pyrrolidin-1-yl]methanone;
[3-[(4,4-Difluorocyclohexyl)methoxy]azetidin-1-yl]-[3-(4H-1,2,4-triazol-3-yl)pyrrolidin-1-yl]methanone;
N-[2-[(3S)-3-(1H-1,2,4-Triazol-5-yl)pyrrolidine-1-carbonyl]-2-azaspiro[3.3]heptan-6-yl]-1-(trifluoromethyl)cyclopropanecarboxamide;
[(3S)-3-(1H-1,2,4-Triazol-5-yl)pyrrolidin-1-yl]-[3-[4-[[1-(trifluoromethyl)cyclopropyl]amino]phenyl]azetidin-1-yl]methanone;
1-[4-[1-[(3S)-3-(1H-1,2,4-Triazol-5-yl)pyrrolidine-1-carbonyl]azetidin-3-yl]phenyl]cyclopropanecarbonitrile;
[(3S)-3-(1H-1,2,4-Triazol-5-yl)pyrrolidin-1-yl]-[3-[6-[1-(trifluoromethyl)cyclopropyl]-3-pyridyl]azetidin-1-yl]methanone;
[(3S)-3-(1H-1,2,4-Triazol-5-yl)pyrrolidin-1-yl]-[3-[2-[1-(trifluoromethyl)cyclopropyl]pyrimidin-5-yl]azetidin-1-yl]methanone;
[3-[(2-Fluoro-4-methylsulfonyl-phenyl)methoxy]azetidin-1-yl]-[(3S)-3-(1H-1,2,4-triazol-5-yl)pyrrolidin-1-yl]methanone;
[(3S)-3-(1H-1,2,4-Triazol-5-yl)pyrrolidin-1-yl]-[6-[[6-(trifluoromethyl)-3-pyridyl]oxy]-2-azaspiro[3.3]heptan-2-yl]methanone;
[(3S)-3-(1H-1,2,4-Triazol-5-yl)pyrrolidin-1-yl]-[[6-[1-(trifluoromethyl)cyclopropyl]methoxy]-2-azaspiro[3.3]heptan-2-yl]methanone;
[(3S)-3-(1H-1,2,4-Triazol-5-yl)pyrrolidin-1-yl]-[2-[2-(trifluoromethyl)phenyl]sulfonyl-2,6-diazaspiro[3.3]heptan-6-yl]methanone;
[(3S)-3-(1H-1,2,4-Triazol-5-yl)pyrrolidin-1-yl]-[2-[3-(trifluoromethoxy)phenyl]sulfonyl-2,6-diazaspiro[3.3]heptan-6-yl]methanone;
N-Methyl-2-[[2-[(3S)-3-(1H-1,2,4-triazol-5-yl)pyrrolidine-1-carbonyl]-2,6-diazaspiro[3.3]heptan-6-yl]methyl]benzamide;
[(3S)-3-(1H-1,2,4-Triazol-5-yl)pyrrolidin-1-yl]-[2-[3-(trifluoromethyl)phenyl]sulfonyl-2,6-diazaspiro[3.3]heptan-6-yl]methanone;
[(3S)-3-(1,2,4-Triazol-4-yl)pyrrolidin-1-yl]-[3-[2-[1-(trifluoromethyl)cyclopropyl]pyrimidin-5-yl]azetidin-1-yl]methanone;
N-[[1-[(3S)-3-(1,2,4-Triazol-4-yl)pyrrolidine-1-carbonyl]azetidin-3-yl]methyl]-3-(trifluoromethyl)benzenesulfonamide;
[3-[4-(3-Dimethylphosphorylphenyl)phenyl]azetidin-1-yl]-[(3S)-3-(1,2,4-triazol-4-yl)pyrrolidin-1-yl]methanone;
[(3S)-3-(1H-Triazol-5-yl)pyrrolidin-1-yl]-[3-[6-(trifluoromethyl)pyridazin-3-yl]oxyazetidin-1-yl]methanone;
1-[3-[4-(4-Chloro-2-methylsulfonyl-phenyl)phenyl]azetidine-1-carbonyl]pyrrolidine-3-sulfonamide;
tert-Butyl N-[(3S)-1-[3-[4-(4-chloro-2-methylsulfonyl-phenyl)phenyl]azetidine-1-carbonyl]pyrrolidin-3-yl]carbamate;
(3S)-1-[3-[5-(2-Chloro-4-methylsulfonyl-phenyl)-2-pyridyl]azetidine-1-carbonyl]pyrrolidine-3-carboxamide;
N-[1-[[(3S)-3-(1H-1,2,4-Triazol-5-yl)pyrrolidine-1-carbonyl]azetidin-3-yl]methyl]-3-(trifluoromethyl)benzenesulfonamide;
N-[[1-[(3S)-3-(1H-1,2,4-Triazol-5-yl)pyrrolidine-1-carbonyl]azetidin-3-yl]methyl]-4-(trifluoromethyl)benzenesulfonamide;
[(3S)-3-(1H-1,2,4-Triazol-5-yl)pyrrolidin-1-yl]-[2-[4-(trifluoromethoxy)phenyl]sulfonyl-2,6-diazaspiro[3.3]heptan-6-yl]methanone;
[3-[5-(2,4-Difluorophenyl)-4H-1,2,4-triazol-3-yl]azetidin-1-yl]-[(3S)-3-(1H-1,2,4-triazol-5-yl)pyrrolidin-1-yl]methanone;
(3S)-1-[3-[5-(4-Chloro-2-methylsulfonyl-phenyl)-2-pyridyl]azetidine-1-carbonyl]pyrrolidine-3-carboxamide;
(3S)-1-[3-[5-(2-Chloro-4-methylsulfonyl-phenyl)pyrazin-2-yl]azetidine-1-carbonyl]pyrrolidine-3-carboxamide;
(3R)-1-[3-[[2-Fluoro-4-(trifluoromethyl)phenyl]methoxy]azetidine-1-carbonyl]pyrrolidine-3-carboxamide;
(3R)-1-[6-[[6-(Trifluoromethyl)-3-pyridyl]oxy]-2-azaspiro[3.3]heptane-2-carbonyl]pyrrolidine-3-carboxamide;
(3S)-1-[3-[[2-Fluoro-4-(trifluoromethyl)phenyl]methylamino]azetidine-1-carbonyl]pyrrolidine-3-carboxamide;
[3-[[2-Fluoro-4-(trifluoromethyl)phenyl]methylamino]azetidin-1-yl]-[(3S)-3-(1H-1,2,4-triazol-5-yl)pyrrolidin-1-yl]methanone;
[3-[[2-Fluoro-4-(trifluoromethyl)phenyl]methylamino]azetidin-1-yl]-[(3R)-3-(1H-1,2,4-triazol-5-yl)pyrrolidin-1-yl]methanone;
2-[[(3S)-3-(1H-1,2,4-Triazol-5-yl)pyrrolidine-1-carbonyl]-N-[1-(trifluoromethyl)cyclopropyl]methyl]-2-azaspiro[3.3]heptane-6-carboxamide;
[(3S)-3-(1,2,4-Triazol-4-yl)pyrrolidin-1-yl]-[[6-[[1-(trifluoromethyl)cyclopropyl]amino]methyl]-2-azaspiro[3.3]heptan-2-yl]methanone;
[3-[[2-Fluoro-4-(trifluoromethyl)phenyl]methylamino]azetidin-1-yl]-[(3R)-3-(tetrazol-1-yl)pyrrolidin-1-yl]methanone;
[3-[4-(Benzenesulfonyl)phenyl]azetidin-1-yl]-[(3S)-3-(tetrazol-1-yl)pyrrolidin-1-yl]methanone;
[(3S)-3-(Tetrazol-1-yl)pyrrolidin-1-yl]-[3-[4-[4-(trifluoromethyl)pyrimidin-2-yl]oxyphenyl]azetidin-1-yl]methanone;
[3-[6-(2-Chlorophenoxy)pyridazin-3-yl]azetidin-1-yl]-[(3S)-3-(tetrazol-1-yl)pyrrolidin-1-yl]methanone;
[3-[[2-Fluoro-4-(trifluoromethyl)phenyl]methylamino]azetidin-1-yl]-[(3S)-3-(tetrazol-1-yl)pyrrolidin-1-yl]methanone;
Methyl 2-methyl-2-[3-[1-[(3S)-3-(tetrazol-1-yl)pyrrolidine-1-carbonyl]azetidin-3-yl]oxyphenyl]propanoate;
[2-[4-Fluoro-2-(trifluoromethyl)phenyl]sulfonyl-2,6-diazaspiro[3.3]heptan-6-yl]-[(3S)-3-(tetrazol-1-yl)pyrrolidin-1-yl]methanone;
Methyl 2-methyl-2-[3-[1-[(3R)-3-(tetrazol-1-yl)pyrrolidine-1-carbonyl]azetidin-3-yl]oxyphenyl]propanoate;
[(3R)-3-(Tetrazol-1-yl)pyrrolidin-1-yl]-[3-[4-[4-(trifluoromethyl)pyrimidin-2-yl]oxyphenyl]azetidin-1-yl]methanone;
[2-[4-Fluoro-3-(trifluoromethyl)phenyl]sulfonyl-2,6-diazaspiro[3.3]heptan-6-yl]-[(3S)-3-(tetrazol-1-yl)pyrrolidin-1-yl]methanone;
[3-[(2-Fluoro-4-methylsulfonyl-phenyl)methoxy]azetidin-1-yl]-[(3S)-3-(tetrazol-1-yl)pyrrolidin-1-yl]methanone;
[2-[4-Fluoro-2-(trifluoromethyl)phenyl]sulfonyl-2,6-diazaspiro[3.3]heptan-6-yl]-[(3R)-3-(tetrazol-1-yl)pyrrolidin-1-yl]methanone;
[3-[4-(4-Chloro-2-methylsulfonyl-phenyl)phenyl]azetidin-1-yl]-[(3S)-3-(tetrazol-1-yl)pyrrolidin-1-yl]methanone;
[3-[4-(Dimethylphosphorylmethyl)phenyl]azetidin-1-yl]-[(3S)-3-(1H-triazol-5-yl)pyrrolidin-1-yl]methanone;
[7-[2-Fluoro-4-(trifluoromethyl) phenoxy]-2-azaspiro[3.5]nonan-2-yl]-[(3S)-3-(1,2,4-triazol-4-yl)pyrrolidin-1-yl]methanone;
(3S)-1-[3-[4-(2-Chloro-4-methylsulfonyl-phenyl)phenyl]azetidine-1-carbonyl]pyrrolidine-3-carboxamide;
[3-[4-(2-Chloro-4-methylsulfonyl-phenyl)phenyl]azetidin-1-yl]-[(3S)-3-hydroxypyrrolidin-1-yl]methanone;
[3-[4-(4-Chloro-2-methylsulfonyl-phenyl)phenyl]azetidin-1-yl]-[(3S)-3-hydroxypyrrolidin-1-yl]methanone;
(3S)-1-[3-[4-(6-Chloro-4-methylsulfonyl-3-pyridyl)phenyl]azetidine-1-carbonyl]pyrrolidine-3-carboxamide;
[3-[4-(4-Chloro-2-methylsulfonyl-phenyl)phenyl]azetidin-1-yl]-[(3S)-3-(1H-1,2,4-triazol-5-yl)pyrrolidin-1-yl]methanone;
(3-Pyrrolidin-1-ylsulfonylpyrrolidin-1-yl)-[3-[4-[1-(trifluoromethyl)cyclopropyl]phenyl]azetidin-1-yl]methanone;
Morpholino-[3-[4-[1-(trifluoromethyl)cyclopropyl]phenyl]azetidin-1-yl]methanone;
[3-[5-(4-Chloro-2-fluoro-phenoxy) pyrazin-2-yl]azetidin-1-yl]-morpholino-methanone;
[3-[[rac-(1S,5R)-6,6-difluoro-3-bicyclo[3.1.0]hexanyl]methoxy]azetidin-1-yl]-[3-(4H-1,2,4-triazol-3-yl)pyrrolidin-1-yl]methanone;
[6-[[2-Fluoro-4-(trifluoromethyl)phenyl]methylamino]-2-azaspiro[3.3]heptan-2-yl]-[(3S)-3-(4H-1,2,4-triazol-3-yl)pyrrolidin-1-yl]methanone;
[7-[[2-Fluoro-4-(trifluoromethyl)phenyl]methyl]-2,7-diazaspiro[3.5]nonan-2-yl]-[(3S)-3-(4H-1,2,4-triazol-3-yl)pyrrolidin-1-yl]methanone;
[7-[[2-Fluoro-4-(trifluoromethyl)phenyl]methyl]-2,7-diazaspiro[3.5]nonan-2-yl]-[(3R)-3-(4H-1,2,4-triazol-3-yl)pyrrolidin-1-yl]methanone;
[7-[2-Fluoro-4-(trifluoromethyl) phenoxy]-2-azaspiro[3.5]nonan-2-yl]-[(3S)-3-(4H-1,2,4-triazol-3-yl)pyrrolidin-1-yl]methanone;
[7-[2-Fluoro-4-(trifluoromethyl) phenoxy]-2-azaspiro[3.5]nonan-2-yl]-[(3R)-3-(4H-1,2,4-triazol-3-yl)pyrrolidin-1-yl]methanone;
(3S)-1-[3-[5-(4-Chloro-2-fluoro-phenyl)pyrimidin-2-yl]azetidine-1-carbonyl]pyrrolidine-3-carboxamide;
(3S)-1-[3-[5-(4-Chloro-2-fluoro-phenyl)-2-pyridyl]azetidine-1-carbonyl]pyrrolidine-3-carboxamide;
(3S)-1-[3-[5-(2,4-Dichlorophenyl)-2-pyridyl]azetidine-1-carbonyl]pyrrolidine-3-carboxamide;
(3S)-1-[3-[4-(5-Chloro-3-methylsulfonyl-2-pyridyl)phenyl]azetidine-1-carbonyl]pyrrolidine-3-carboxamide;
(3S)-1-[3-[6-(2-Chloro-4-methylsulfonyl-phenyl)-3-pyridyl]azetidine-1-carbonyl]pyrrolidine-3-carboxamide;
(3S)-1-[3-[6-(4-Chloro-2-methylsulfonyl-phenyl)-3-pyridyl]azetidine-1-carbonyl]pyrrolidine-3-carboxamide;
[3-[4-(6-Chloro-4-methylsulfonyl-3-pyridyl)phenyl]azetidin-1-yl]-[(3S)-3-hydroxypyrrolidin-1-yl]methanone;
(3S)-1-[3-[4-[5-(Trifluoromethyl)pyrazin-2-yl]phenyl]azetidine-1-carbonyl]pyrrolidine-3-carboxamide;
[(3S)-3-Aminopyrrolidin-1-yl]-[3-[4-(4-chloro-2-methylsulfonyl-phenyl)phenyl]azetidin-1-yl]methanone;
[2-[[2-Fluoro-4-(trifluoromethyl)phenyl]methyl]-2,7-diazaspiro[3.5]nonan-7-yl]-[(3S)-3-(4H-1,2,4-triazol-3-yl)pyrrolidin-1-yl]methanone;
[2-[[2-Fluoro-4-(trifluoromethyl)phenyl]methyl]-2,7-diazaspiro[3.5]nonan-7-yl]-[(3R)-3-(4H-1,2,4-triazol-3-yl)pyrrolidin-1-yl]methanone;
(3S)-1-[7-[[2-Fluoro-4-(trifluoromethyl)phenyl]methyl]-2,7-diazaspiro[3.5]nonane-2-carbonyl]pyrrolidine-3-carboxamide;
(3R)-1-[7-[[2-Fluoro-4-(trifluoromethyl)phenyl]methyl]-2,7-diazaspiro[3.5]nonane-2-carbonyl]pyrrolidine-3-carboxamide;
(3S)-1-[7-[2-Fluoro-4-(trifluoromethyl) phenoxy]-2-azaspiro[3.5]nonane-2-carbonyl]pyrrolidine-3-carboxamide;
(3R)-1-[7-[2-Fluoro-4-(trifluoromethyl) phenoxy]-2-azaspiro[3.5]nonane-2-carbonyl]pyrrolidine-3-carboxamide;
(3S)-1-[2-[[2-Fluoro-4-(trifluoromethyl)phenyl]methyl]-2,7-diazaspiro[3.5]nonane-7-carbonyl]pyrrolidine-3-carboxamide;
(3R)-1-[2-[[2-Fluoro-4-(trifluoromethyl)phenyl]methyl]-2,7-diazaspiro[3.5]nonane-7-carbonyl]pyrrolidine-3-carboxamide;
[7-[2-Fluoro-4-(trifluoromethyl)phenyl]sulfonyl-2,7-diazaspiro[3.5]nonan-2-yl]-[(3S)-3-(1H-1,2,4-triazol-5-yl)pyrrolidin-1-yl]methanone;
(3S)-1-[7-[2-Fluoro-4-(trifluoromethyl)phenyl]sulfonyl-2,7-diazaspiro[3.5]nonane-2-carbonyl]pyrrolidine-3-carboxamide;
[6-[(3,5-Difluoro-2-pyridyl)methyl]-2-azaspiro[3.3]heptan-2-yl]-[(3S)-3-(tetrazol-1-yl)pyrrolidin-1-yl]methanone;
[(3S)-3-(Tetrazol-1-yl)pyrrolidin-1-yl]-[6-[[6-(trifluoromethyl)-3-pyridyl]methyl]-2-azaspiro[3.3]heptan-2-yl]methanone;
[6-[(5-Fluoro-3-pyridyl)methyl]-2-azaspiro[3.3]heptan-2-yl]-[(3S)-3-(tetrazol-1-yl)pyrrolidin-1-yl]methanone;
[6-[(3,5-Difluoro-2-pyridyl)methyl]-2-azaspiro[3.3]heptan-2-yl]-[(3R)-3-(tetrazol-1-yl)pyrrolidin-1-yl]methanone;
[(3R)-3-(Tetrazol-1-yl)pyrrolidin-1-yl]-[6-[[6-(trifluoromethyl)-3-pyridyl]methyl]-2-azaspiro[3.3]heptan-2-yl]methanone;
[6-[(5-Fluoro-3-pyridyl)methyl]-2-azaspiro[3.3]heptan-2-yl]-[(3R)-3-(tetrazol-1-yl)pyrrolidin-1-yl]methanone;
[3-(4H-1,2,4-Triazol-3-yl)pyrrolidin-1-yl]-[7-[2-(trifluoromethyl)pyrimidin-4-yl]oxy-2-azaspiro[3.5]nonan-2-yl]methanone;
N-[2-[3-(4H-1,2,4-Triazol-3-yl)pyrrolidine-1-carbonyl]-2-azaspiro[3.5]nonan-7-yl]-3-(trifluoromethoxy)benzenesulfonamide;
(3S)-1-[3-[4-(4-Chloro-2-fluoro-phenyl)-3-methylsulfonyl-phenyl]azetidine-1-carbonyl]pyrrolidine-3-carboxamide;
N-[2-[(3S)-3-(1H-1,2,4-Triazol-5-yl)pyrrolidine-1-carbonyl]-2-azaspiro[3.3]heptan-6-yl]-2-(trifluoromethyl)benzenesulfonamide;
N-[2-[(3S)-3-(1H-1,2,4-Triazol-5-yl)pyrrolidine-1-carbonyl]-2-azaspiro[3.3]heptan-6-yl]-3-(trifluoromethyl)benzenesulfonamide;
N-[2-[(3S)-3-(1H-1,2,4-Triazol-5-yl)pyrrolidine-1-carbonyl]-2-azaspiro[3.3]heptan-6-yl]-4-(trifluoromethyl)benzenesulfonamide;
[(3S)-3-(1H-1,2,4-Triazol-5-yl)pyrrolidin-1-yl]-[6-[[5-(trifluoromethyl)-2-pyridyl]methyl]-2-azaspiro[3.3]heptan-2-yl]methanone;
[(3S)-3-(1H-1,2,4-Triazol-5-yl)pyrrolidin-1-yl]-[6-[[6-(trifluoromethyl)-3-pyridyl]methyl]-2-azaspiro[3.3]heptan-2-yl]methanone;
[(3S)-3-(1H-1,2,4-Triazol-5-yl)pyrrolidin-1-yl]-[6-[[5-(trifluoromethyl)pyrazin-2-yl]methyl]-2-azaspiro[3.3]heptan-2-yl]methanone;
(3S)-1-[6-[[2-(Trifluoromethyl)phenyl]sulfonylamino]-2-azaspiro[3.3]heptane-2-carbonyl]pyrrolidine-3-carboxamide;
(3S)-1-[6-[[3-(Trifluoromethyl)phenyl]sulfonylamino]-2-azaspiro[3.3]heptane-2-carbonyl]pyrrolidine-3-carboxamide;
(3S)-1-[6-[[4-(Trifluoromethyl)phenyl]sulfonylamino]-2-azaspiro[3.3]heptane-2-carbonyl]pyrrolidine-3-carboxamide;
(3S)-1-[6-(2,2-Dimethylpropylsulfonylamino)-2-azaspiro[3.3]heptane-2-carbonyl]pyrrolidine-3-carboxamide;
(3S)-1-[3-[5-(2-Chloro-4-methylsulfonyl-phenyl)pyrimidin-2-yl]azetidine-1-carbonyl]pyrrolidine-3-carboxamide;
(3S)-1-[3-[2-(2-Chloro-4-methylsulfonyl-phenyl)pyrimidin-5-yl]azetidine-1-carbonyl]pyrrolidine-3-carboxamide;
[3-[6-(1,1-Dioxo-1,4-thiazinan-4-yl)-3-pyridyl]azetidin-1-yl]-[3-(1H-1,2,4-triazol-5-yl)pyrrolidin-1-yl]methanone;
[3-[5-(4-Chloro-2-methylsulfonyl-phenyl)-2-pyridyl]azetidin-1-yl]-[(3R)-3-hydroxypyrrolidin-1-yl]methanone;
[3-(4H-1,2,4-Triazol-3-yl)pyrrolidin-1-yl]-[6-[[6-(trifluoromethyl)-3-pyridyl]methyl]-2-azaspiro[3.3]heptan-2-yl]methanone;
1-[6-[[6-(Trifluoromethyl)-3-pyridyl]methyl]-2-azaspiro[3.3]heptane-2-carbonyl]pyrrolidine-3-sulfonamide;
1-[6-[[6-(Trifluoromethyl)-3-pyridyl]methyl]-2-azaspiro[3.3]heptane-2-carbonyl]piperidine-3-carboxamide;
[3-[5-(4-Chlorophenoxy) pyrazin-2-yl]azetidin-1-yl]-[(3S)-3-(tetrazol-1-yl)pyrrolidin-1-yl]methanone;
[(3S)-3-(Tetrazol-1-yl)pyrrolidin-1-yl]-[6-[4-(trifluoromethyl)phenyl]-2-azaspiro[3.3]heptan-2-yl]methanone;
[(3S)-3-(Tetrazol-1-yl)pyrrolidin-1-yl]-[6-[2-(trifluoromethyl)pyrimidin-5-yl]-2-azaspiro[3.3]heptan-2-yl]methanone;
[3-(4H-1,2,4-Triazol-3-yl)pyrrolidin-1-yl]-[7-[6-(trifluoromethyl)pyrazin-2-yl]oxy-2-azaspiro[3.5]nonan-2-yl]methanone;
[7-(4-Fluoro-2-methylsulfonyl-phenoxy)-2-azaspiro[3.5]nonan-2-yl]-[3-(4H-1,2,4-triazol-3-yl)pyrrolidin-1-yl]methanone;
[(3S)-3-(1,2,4-Triazol-4-yl)pyrrolidin-1-yl]-[3-[[6-[1-(trifluoromethyl)cyclopropyl]methylamino]-3-pyridyl]azetidin-1-yl]methanone;
[3-(4H-1,2,4-Triazol-3-yl)pyrrolidin-1-yl]-[7-[6-(trifluoromethyl)pyridazin-3-yl]oxy-2-azaspiro[3.5]nonan-2-yl]methanone;
[3-(4H-1,2,4-Triazol-3-yl)pyrrolidin-1-yl]-[7-[5-(trifluoromethyl)pyrazin-2-yl]oxy-2-azaspiro[3.5]nonan-2-yl]methanone;
[(3S)-3-(1,2,4-Triazol-4-yl)pyrrolidin-1-yl]-[7-[5-(trifluoromethyl)pyrazin-2-yl]oxy-2-azaspiro[3.5]nonan-2-yl]methanone;
(3R)-1-[3-[4-(4-Chloro-2-methylsulfonyl-phenyl)phenyl]azetidine-1-carbonyl]pyrrolidine-3-sulfonamide;
(3S)-1-[3-[4-(4-Chloro-2-methylsulfonyl-phenyl)phenyl]azetidine-1-carbonyl]pyrrolidine-3-sulfonamide;
3-[3-[4-(4-Chloro-2-methylsulfonyl-phenyl)phenyl]azetidine-1-carbonyl]-4,5-dihydroisoxazole-5-carboxamide;
1-[7-[[2-Fluoro-4-(trifluoromethyl)phenyl]methyl]-2,7-diazaspiro[3.5]nonane-2-carbonyl]piperidine-3-carboxamide;
1-[7-[[2-Fluoro-4-(trifluoromethyl)phenyl]methyl]-2,7-diazaspiro[3.5]nonane-2-carbonyl]pyrrolidine-3-sulfonamide;
[6-[(3-Methylsulfonylphenyl)methyl]-2-azaspiro[3.3]heptan-2-yl]-[(3S)-3-(1H-1,2,4-triazol-5-yl)pyrrolidin-1-yl]methanone;
[6-[(4-Methylsulfonylphenyl)methyl]-2-azaspiro[3.3]heptan-2-yl]-[(3S)-3-(1H-1,2,4-triazol-5-yl)pyrrolidin-1-yl]methanone;
(3S)-1-[6-[(3-Methylsulfonylphenyl)methyl]-2-azaspiro[3.3]heptane-2-carbonyl]pyrrolidine-3-carboxamide;
(3S)-1-[6-[(4-Methylsulfonylphenyl)methyl]-2-azaspiro[3.3]heptane-2-carbonyl]pyrrolidine-3-carboxamide;
[(3S)-3-(1H-1,2,4-Triazol-5-yl)pyrrolidin-1-yl]-[3-[[6-[1-(trifluoromethyl)cyclopropyl]methylamino]-3-pyridyl]azetidin-1-yl]methanone;
(3S)-1-[3-[[6-[1-(Trifluoromethyl)cyclopropyl]methylamino]-3-pyridyl]azetidine-1-carbonyl]pyrrolidine-3-carboxamide;
4-Fluoro-N-[1-[(3S)-3-(1H-1,2,4-triazol-5-yl)pyrrolidine-1-carbonyl]-4-piperidyl]benzenesulfonamide;
N-[1-[(3S)-3-(1H-1,2,4-Triazol-5-yl)pyrrolidine-1-carbonyl]-4-piperidyl]-4-(trifluoromethyl)benzenesulfonamide;
(3S)-1-[4-[[4-(Trifluoromethyl)phenyl]sulfonylamino]piperidine-1-carbonyl]pyrrolidine-3-carboxamide;
4-Chloro-N-[1-[[(3S)-3-(1H-1,2,4-triazol-5-yl)pyrrolidine-1-carbonyl]-4-piperidyl]methyl]benzenesulfonamide;
(3S)-1-[7-(4-Fluoro-2-methylsulfonyl-phenoxy)-2-azaspiro[3.5]nonane-2-carbonyl]pyrrolidine-3-carboxamide;
(3S)-1-[7-[6-(Trifluoromethyl)pyridazin-3-yl]oxy-2-azaspiro[3.5]nonane-2-carbonyl]pyrrolidine-3-carboxamide;
(3S)-1-[7-[2-(Trifluoromethyl)pyrimidin-4-yl]oxy-2-azaspiro[3.5]nonane-2-carbonyl]pyrrolidine-3-carboxamide;
(3S)-1-[7-[[3-(Trifluoromethoxy)phenyl]sulfonylamino]-2-azaspiro[3.5]nonane-2-carbonyl]pyrrolidine-3-carboxamide;
N-[2-[(3S)-3-(1H-1,2,4-Triazol-5-yl)pyrrolidine-1-carbonyl]-2-azaspiro[3.5]nonan-7-yl]-3-(trifluoromethoxy)benzenesulfonamide;
(5S)-3-[3-[4-(4-Chloro-2-methylsulfonyl-phenyl)phenyl]azetidine-1-carbonyl]-4,5-dihydroisoxazole-5-carboxamide;
(5R)-3-[3-[4-(4-Chloro-2-methylsulfonyl-phenyl)phenyl]azetidine-1-carbonyl]-4,5-dihydroisoxazole-5-carboxamide;
1-[3-[4-(4-Chloro-2-methylsulfonyl-phenyl)phenyl]azetidine-1-carbonyl]azetidine-3-sulfonamide;
1-[3-[4-(4-Chloro-2-methylsulfonyl-phenyl)phenyl]azetidine-1-carbonyl]piperidine-4-sulfonamide;
N-[1-[[(3S)-3-(1H-1,2,4-Triazol-5-yl)pyrrolidine-1-carbonyl]-4-piperidyl]methyl]-4-(trifluoromethyl)benzenesulfonamide;
(3S)-1-[4-[[[4-(Trifluoromethyl)phenyl]sulfonylamino]methyl]piperidine-1-carbonyl]pyrrolidine-3-carboxamide;
N-[1-[[(3S)-3-(1H-1,2,4-Triazol-5-yl)pyrrolidine-1-carbonyl]-4-piperidyl]methyl]-4-(trifluoromethoxy)benzenesulfonamide;
(3S)-1-[4-[[[4-(Trifluoromethoxy)phenyl]sulfonylamino]methyl]piperidine-1-carbonyl]pyrrolidine-3-carboxamide;
(3S)-1-[3-[4-(6,6-Difluoro-2-azaspiro[3.3]heptan-2-yl)phenyl]azetidine-1-carbonyl]pyrrolidine-3-carboxamide;
(3S)-1-[3-[4-[3-(Trifluoromethyl)cyclobutyl]phenyl]azetidine-1-carbonyl]pyrrolidine-3-carboxamide;
(3S)-1-[3-[4-(2-Azaspiro[3.4]octan-2-yl)phenyl]azetidine-1-carbonyl]pyrrolidine-3-carboxamide;
[(3S)-3-(1H-1,2,4-Triazol-5-yl)pyrrolidin-1-yl]-[6-[[1-(trifluoromethyl)cyclopropyl]methylamino]-2-azaspiro[3.3]heptan-2-yl]methanone;
[6-[(4-Fluoro-2-methylsulfonyl-phenyl)methyl]-2-azaspiro[3.3]heptan-2-yl]-[(3S)-3-(1H-1,2,4-triazol-5-yl)pyrrolidin-1-yl]methanone;
[3-[6-(3-Hydroxy-3-methyl-azetidin-1-yl)-3-pyridyl]azetidin-1-yl]-[(3S)-3-(1H-1,2,4-triazol-5-yl)pyrrolidin-1-yl]methanone;
[3-[6-[3-Hydroxy-3-(trifluoromethyl)azetidin-1-yl]-3-pyridyl]azetidin-1-yl]-[(3S)-3-(1H-1,2,4-triazol-5-yl)pyrrolidin-1-yl]methanone;
[3-[6-[3-Hydroxy-3-(trifluoromethyl)pyrrolidin-1-yl]-3-pyridyl]azetidin-1-yl]-[rac-(3S)-3-(1H-1,2,4-triazol-5-yl)pyrrolidin-1-yl]methanone;
[3-[4-[1-(1H-Tetrazol-5-yl)cyclopropyl]phenyl]azetidin-1-yl]-[(3S)-3-(1H-1,2,4-triazol-5-yl)pyrrolidin-1-yl]methanone;
[3-[4-[1-(2-tert-Butyltetrazol-5-yl)cyclopropyl]phenyl]azetidin-1-yl]-[(3S)-3-(1H-1,2,4-triazol-5-yl)pyrrolidin-1-yl]methanone;
(3S)-1-[6-[[1-(Trifluoromethyl)cyclopropyl]methylamino]-2-azaspiro[3.3]heptane-2-carbonyl]pyrrolidine-3-carboxamide;
(3S)-1-[6-[(4-Fluoro-2-methylsulfonyl-phenyl)methyl]-2-azaspiro[3.3]heptane-2-carbonyl]pyrrolidine-3-carboxamide;
(3S)-1-[3-[6-[3-Hydroxy-3-(trifluoromethyl)azetidin-1-yl]-3-pyridyl]azetidine-1-carbonyl]pyrrolidine-3-carboxamide;
rac-(3S)-1-[3-[6-[3-Hydroxy-3-(trifluoromethyl)pyrrolidin-1-yl]-3-pyridyl]azetidine-1-carbonyl]pyrrolidine-3-carboxamide;
(3S)-1-[3-[4-[1-(2-tert-Butyltetrazol-5-yl)cyclopropyl]phenyl]azetidine-1-carbonyl]pyrrolidine-3-carboxamide;
(3S)-1-[6-[[1-(Trifluoromethyl)cyclopropyl]amino]-2-azaspiro[3.3]heptane-2-carbonyl]pyrrolidine-3-carboxamide;
[(3S)-3-(1H-1,2,4-Triazol-5-yl)pyrrolidin-1-yl]-[6-[[1-(trifluoromethyl)cyclopropyl]amino]-2-azaspiro[3.3]heptan-2-yl]methanone;
(3S)-1-[3-[6-[(3R)-3-Hydroxy-3-(trifluoromethyl)pyrrolidin-1-yl]-3-pyridyl]azetidine-1-carbonyl]pyrrolidine-3-carboxamide;
(3S)-1-[3-[6-[(3S)-3-Hydroxy-3-(trifluoromethyl)pyrrolidin-1-yl]-3-pyridyl]azetidine-1-carbonyl]pyrrolidine-3-carboxamide;
[3-[4-(4-Fluorophenoxy)phenyl]azetidin-1-yl]-[(3R)-3-(tetrazol-2-yl)pyrrolidin-1-yl]methanone;
[(3R)-3-(Tetrazol-2-yl)pyrrolidin-1-yl]-[3-[4-[1-(trifluoromethyl)cyclopropyl]phenyl]azetidin-1-yl]methanone;
[3-[6-[(3R)-3-Hydroxy-3-(trifluoromethyl)pyrrolidin-1-yl]-3-pyridyl]azetidin-1-yl]-[(3S)-3-(1H-1,2,4-triazol-5-yl)pyrrolidin-1-yl]methanone;
[3-[6-[(3S)-3-Hydroxy-3-(trifluoromethyl)pyrrolidin-1-yl]-3-pyridyl]azetidin-1-yl]-[(3S)-3-(1H-1,2,4-triazol-5-yl)pyrrolidin-1-yl]methanone;
[3-[6-(4-Chloro-2-methylsulfonyl-phenoxy)-3-pyridyl]azetidin-1-yl]-[(3S)-3-(1,2,4-triazol-4-yl)pyrrolidin-1-yl]methanone;
1-[3-[4-[1-(Trifluoromethyl)cyclopropyl]phenyl]azetidine-1-carbonyl]pyrrolidine-3-sulfonamide;
1-[3-[[2-Fluoro-4-(trifluoromethyl)phenyl]methoxy]azetidine-1-carbonyl]pyrrolidine-3-sulfonamide;
[(3S)-3-(1H-1,2,4-Triazol-5-yl)pyrrolidin-1-yl]-[3-[[4-(trifluoromethylsulfonyl)phenyl]methoxy]azetidin-1-yl]methanone;
[(3S)-3-(1H-1,2,4-Triazol-5-yl)pyrrolidin-1-yl]-[3-[[3-(trifluoromethylsulfonyl)phenyl]methoxy]azetidin-1-yl]methanone;
Methyl 2-[4-[1-[(3S)-3-(1H-1,2,4-triazol-5-yl)pyrrolidine-1-carbonyl]azetidin-3-yl]phenyl]benzoate;
1-[4-[1-[rac-(3S)-3-(1H-1,2,4-Triazol-5-yl)pyrrolidine-1-carbonyl]azetidin-3-yl]phenyl]piperidine-2-carboxamide;
[3-[[2-Fluoro-5-(trifluoromethyl)phenyl]methylamino]azetidin-1-yl]-[(3S)-3-(1H-1,2,4-triazol-5-yl)pyrrolidin-1-yl]methanone;
[(3S)-3-(1H-1,2,4-Triazol-5-yl)pyrrolidin-1-yl]-[3-[[4-(trifluoromethyl)phenyl]methylamino]azetidin-1-yl]methanone;
[(3S)-3-(Tetrazol-1-yl)pyrrolidin-1-yl]-[3-[2-[1-(trifluoromethyl)cyclopropyl]pyrimidin-5-yl]azetidin-1-yl]methanone;
(3S)-1-[7-[[5-(Trifluoromethyl)pyrazin-2-yl]amino]-2-azaspiro[3.5]nonane-2-carbonyl]pyrrolidine-3-carboxamide;
(3R)-1-[7-[[5-(Trifluoromethyl)pyrazin-2-yl]amino]-2-azaspiro[3.5]nonane-2-carbonyl]pyrrolidine-3-carboxamide;
(3S)-1-[7-[5-(Trifluoromethyl)pyrimidin-2-yl]oxy-2-azaspiro[3.5]nonane-2-carbonyl]pyrrolidine-3-carboxamide;
[3-[4-(4-Fluorophenoxy)phenyl]azetidin-1-yl]-[(3S)-3-(tetrazol-2-yl)pyrrolidin-1-yl]methanone;
[3-[4-(4-Chloro-2-methylsulfonyl-phenyl)phenyl]azetidin-1-yl]-[(3S)-3-(tetrazol-2-yl)pyrrolidin-1-yl]methanone;
[3-[4-(4-Chloro-2-methylsulfonyl-phenyl)phenyl]azetidin-1-yl]-[(3R)-3-(tetrazol-2-yl)pyrrolidin-1-yl]methanone;
[(3S)-3-(Tetrazol-1-yl)pyrrolidin-1-yl]-[3-[4-[[1-(trifluoromethyl)cyclopropyl]methoxy]phenyl]azetidin-1-yl]methanone;
[(3S)-3-(Tetrazol-1-yl)pyrrolidin-1-yl]-[3-[4-[5-(trifluoromethyl)pyrazin-2-yl]oxyphenyl]azetidin-1-yl]methanone;
[(3S)-3-(1H-1,2,4-Triazol-5-yl)pyrrolidin-1-yl]-[3-[[6-[1-(trifluoromethyl)cyclopropyl]amino]-3-pyridyl]azetidin-1-yl]methanone;
4,4-Difluoro-1-[4-[1-[rac-(3S)-3-(1H-1,2,4-triazol-5-yl)pyrrolidine-1-carbonyl]azetidin-3-yl]phenyl]piperidine-2-carboxamide;
(3S)-1-[7-[5-(Trifluoromethyl)pyrazin-2-yl]oxy-2-azaspiro[3.5]nonane-2-carbonyl]pyrrolidine-3-carboxamide;
[3-[[5-Chloro-2-(trifluoromethyl)phenyl]methylamino]azetidin-1-yl]-[(3S)-3-(1H-1,2,4-triazol-5-yl)pyrrolidin-1-yl]methanone;
[3-[[3-Chloro-5-(trifluoromethyl)phenyl]methylamino]azetidin-1-yl]-[(3S)-3-(1H-1,2,4-triazol-5-yl)pyrrolidin-1-yl]methanone;
[3-[[2-Fluoro-4-(trifluoromethoxy)phenyl]methylamino]azetidin-1-yl]-[(3S)-3-(1H-1,2,4-triazol-5-yl)pyrrolidin-1-yl]methanone;
[3-[[3-Fluoro-4-(trifluoromethyl)phenyl]methylamino]azetidin-1-yl]-[(3S)-3-(1H-1,2,4-triazol-5-yl)pyrrolidin-1-yl]methanone;
[3-[[2-Chloro-4-(trifluoromethyl)phenyl]methylamino]azetidin-1-yl]-[(3S)-3-(1H-1,2,4-triazol-5-yl)pyrrolidin-1-yl]methanone;
2-[4-[1-[(3S)-3-(1H-1,2,4-Triazol-5-yl)pyrrolidine-1-carbonyl]azetidin-3-yl]phenyl]benzoic acid;
(3S)-1-[3-[4-(3,5-Dimethylpyrazol-1-yl)phenyl]azetidine-1-carbonyl]pyrrolidine-3-carboxamide;
[6-[(3-Methylsulfonylphenyl)methyl]-2-azaspiro[3.3]heptan-2-yl]-[(3S)-3-(tetrazol-1-yl)pyrrolidin-1-yl]methanone;
[3-[[2-Chloro-4-(trifluoromethyl)phenyl]methylamino]azetidin-1-yl]-[(3S)-3-(tetrazol-1-yl)pyrrolidin-1-yl]methanone;
[3-[[3-Fluoro-4-(trifluoromethyl)phenyl]methylamino]azetidin-1-yl]-[(3S)-3-(tetrazol-1-yl)pyrrolidin-1-yl]methanone;
[3-[[2-Fluoro-4-(trifluoromethoxy)phenyl]methylamino]azetidin-1-yl]-[(3S)-3-(tetrazol-1-yl)pyrrolidin-1-yl]methanone;
[3-[[5-Chloro-2-(trifluoromethyl)phenyl]methylamino]azetidin-1-yl]-[(3S)-3-(tetrazol-1-yl)pyrrolidin-1-yl]methanone;
[3-[[3-Chloro-5-(trifluoromethyl)phenyl]methylamino]azetidin-1-yl]-[(3S)-3-(tetrazol-1-yl)pyrrolidin-1-yl]methanone;
[(3S)-3-(Tetrazol-1-yl)pyrrolidin-1-yl]-[3-[5-[1-(trifluoromethyl)cyclopropyl]-2-pyridyl]azetidin-1-yl]methanone;
(3S)-1-[3-[6-(2-Azaspiro[3.4]octan-2-yl)-3-pyridyl]azetidine-1-carbonyl]pyrrolidine-3-carboxamide;
1-[3-[6-(2-Azaspiro[3.4]octan-2-yl)-3-pyridyl]azetidine-1-carbonyl]pyrrolidine-3-sulfonamide;
1-[4-[1-[(3S)-3-(1H-1,2,4-Triazol-5-yl)pyrrolidine-1-carbonyl]azetidin-3-yl]phenyl]piperidine-2-carboxamide;
(3S)-1-[7-[[6-(Trifluoromethyl)-3-pyridyl]methyl]-2-azaspiro[3.5]nonane-2-carbonyl]pyrrolidine-3-carboxamide;
2-[4-[1-[(3S)-3-(1H-1,2,4-Triazol-5-yl)pyrrolidine-1-carbonyl]azetidin-3-yl]phenyl]benzamide;
(3S)-1-[3-[[2-Fluoro-4-(trifluoromethyl)phenyl]methyl-methyl-amino]azetidine-1-carbonyl]pyrrolidine-3-carboxamide;
(3S)-1-[3-[[4-(Trifluoromethylsulfonyl)phenyl]methoxy]azetidine-1-carbonyl]pyrrolidine-3-carboxamide;
(3S)-1-[3-[[3-(Trifluoromethylsulfonyl)phenyl]methoxy]azetidine-1-carbonyl]pyrrolidine-3-carboxamide;
(3S)-1-[3-[5-[6-(Trifluoromethyl)-3-pyridyl]-1,2,4-oxadiazol-3-yl]azetidine-1-carbonyl]pyrrolidine-3-carboxamide;
(3S)-1-[3-[5-[3-(Trifluoromethyl)-1-bicyclo[1.1.1]pentanyl]-1,2,4-oxadiazol-3-yl]azetidine-1-carbonyl]pyrrolidine-3-carboxamide;
rac-(3S)-1-[3-[6-[3-(Trifluoromethyl)pyrrolidin-1-yl]-3-pyridyl]azetidine-1-carbonyl]pyrrolidine-3-carboxamide;
N-Ethyl-2-[4-[1-[(3S)-3-(1H-1,2,4-triazol-5-yl)pyrrolidine-1-carbonyl]azetidin-3-yl]phenyl]benzamide;
[3-[Cyclopropylmethyl-[[2-fluoro-4-(trifluoromethyl)phenyl]methyl]amino]azetidin-1-yl]-[(3S)-3-(tetrazol-1-yl)pyrrolidin-1-yl]methanone;
[3-[4-(4-Chloro-2-methylsulfonyl-phenyl)phenyl]azetidin-1-yl]-(3-hydroxyazetidin-1-yl)methanone;
[3-[4-(4-Chloro-2-methylsulfonyl-phenyl)phenyl]azetidin-1-yl]-(3-hydroxy-3-methyl-azetidin-1-yl)methanone;
1-[3-[4-(4-Chloro-2-methylsulfonyl-phenyl)phenyl]azetidine-1-carbonyl]azetidine-3-carboxamide;
1-[3-[4-[5-(Trifluoromethyl)pyrimidin-2-yl]oxyphenyl]azetidine-1-carbonyl]azetidine-3-carboxamide;
[3-[[2-Fluoro-4-(trifluoromethyl)phenyl]methyl-methyl-amino]azetidin-1-yl]-[(3S)-3-(tetrazol-1-yl)pyrrolidin-1-yl]methanone;
(3S)-1-[3-(5-Spiro[3.3]heptan-2-ylpyrazin-2-yl)azetidine-1-carbonyl]pyrrolidine-3-carboxamide;
(3S)-1-[3-[4-[4-Methyl-2-(pyrrolidine-1-carbonyl)phenyl]phenyl]azetidine-1-carbonyl]pyrrolidine-3-carboxamide;
(3S)-1-[6-[[6-(Trifluoromethyl)-3-pyridyl]methyl]-2-azaspiro[3.3]heptane-2-carbonyl]pyrrolidine-3-carboxamide;
[3-[4-(4-Chloro-2-methylsulfonyl-phenyl)phenyl]azetidin-1-yl]-[3-(methylsulfonimidoyl)pyrrolidin-1-yl]methanone;
[3-[[2-Fluoro-4-(trifluoromethyl)phenyl]methyl-methyl-amino]azetidin-1-yl]-[(3S)-3-(1,2,4-triazol-4-yl)pyrrolidin-1-yl]methanone;
N-[1-[3-[4-[1-(Trifluoromethyl)cyclopropyl]phenyl]azetidine-1-carbonyl]azetidin-3-yl]methanesulfonamide;
N-[1-[3-[[4-[1-(Trifluoromethyl)cyclopropyl]methoxy]phenyl]azetidine-1-carbonyl]azetidin-3-yl]methanesulfonamide;
N-[1-[3-[4-[5-(Trifluoromethyl)pyrazin-2-yl]oxyphenyl]azetidine-1-carbonyl]azetidin-3-yl]methanesulfonamide;
N-[1-[3-[6-[3-(Trifluoromethyl)azetidin-1-yl]-3-pyridyl]azetidine-1-carbonyl]azetidin-3-yl]methanesulfonamide;
(3S)-1-[3-[3-[5-(2,2-Dimethylpropyl)-1,3,4-oxadiazol-2-yl]-1-bicyclo[1.1.1]pentanyl]azetidine-1-carbonyl]pyrrolidine-3-carboxamide;
[3-[3-[5-(2,2-Dimethylpropyl)-1,3,4-oxadiazol-2-yl]-1-bicyclo[1.1.1]pentanyl]azetidin-1-yl]-[(3S)-3-(1H-1,2,4-triazol-5-yl)pyrrolidin-1-yl]methanone;
(3S)-1-[3-[4-[3-(2,2-Dimethylpropyl)triazol-4-yl]phenyl]azetidine-1-carbonyl]pyrrolidine-3-carboxamide;
rac-(3S)-1-[3-[6-(2,2-Difluoro-5-azaspiro[2.4]heptan-5-yl)-3-pyridyl]azetidine-1-carbonyl]pyrrolidine-3-carboxamide;
[(3S)-3-(Tetrazol-1-yl)pyrrolidin-1-yl]-[6-[[6-(trifluoromethyl)pyridazin-3-yl]methyl]-2,6-diazaspiro[3.3]heptan-2-yl]methanone;
tert-Butyl 2-[3-[[2-fluoro-4-(trifluoromethyl)phenyl]methyl-methyl-amino]azetidine-1-carbonyl]-2,8-diazaspiro[3.5]nonane-8-carboxylate;
N-[1-[2-[[2-Fluoro-4-(trifluoromethyl)phenyl]methyl]-2,7-diazaspiro[3.5]nonane-7-carbonyl]azetidin-3-yl]methanesulfonamide;
(3S)-1-[3-[6-(6-Oxa-2-azaspiro[3.4]octan-2-yl)-3-pyridyl]azetidine-1-carbonyl]pyrrolidine-3-carboxamide;
(3S)-1-[3-[6-(5-Oxa-2-azaspiro[3.4]octan-2-yl)-3-pyridyl]azetidine-1-carbonyl]pyrrolidine-3-carboxamide;
[7-[[6-(Difluoromethoxy)-3-pyridyl]methyl]-2-azaspiro[3.5]nonan-2-yl]-[(3S)-3-(tetrazol-1-yl)pyrrolidin-1-yl]methanone;
[6-[(2,4-Difluorophenyl)methyl]-2-azaspiro[3.3]heptan-2-yl]-[(3S)-3-(tetrazol-1-yl)pyrrolidin-1-yl]methanone;
[6-[[2-Fluoro-4-(trifluoromethyl)phenyl]methyl]-2-azaspiro[3.3]heptan-2-yl]-[(3S)-3-(tetrazol-1-yl)pyrrolidin-1-yl]methanone;
[6-[[4-Fluoro-2-(trifluoromethyl)phenyl]methyl]-2-azaspiro[3.3]heptan-2-yl]-[(3S)-3-(tetrazol-1-yl)pyrrolidin-1-yl]methanone;
N-[1-[3-[4-[1-(Trifluoromethyl)cyclopropyl]phenyl]azetidine-1-carbonyl]azetidin-3-yl]acetamide;
N-[1-[3-[[2-Fluoro-4-(trifluoromethyl)phenyl]methoxy]azetidine-1-carbonyl]azetidin-3-yl]acetamide;
N-[1-[7-[[1-(Trifluoromethyl)cyclopropyl]methoxy]-2-azaspiro[3.5]nonane-2-carbonyl]azetidin-3-yl]acetamide;
N-[1-[3-[4-[5-(Trifluoromethyl)pyrazin-2-yl]oxyphenyl]azetidine-1-carbonyl]azetidin-3-yl]acetamide;
(3S)-1-[3-[6-[(3R)-3-(Trifluoromethyl)pyrrolidin-1-yl]-3-pyridyl]azetidine-1-carbonyl]pyrrolidine-3-carboxamide;
(3S)-1-[3-[6-[(3S)-3-(Trifluoromethyl)pyrrolidin-1-yl]-3-pyridyl]azetidine-1-carbonyl]pyrrolidine-3-carboxamide;
(3S)-1-[3-[4-[5-(Trifluoromethyl)pyrazin-2-yl]oxyphenyl]azetidine-1-carbonyl]pyrrolidine-3-carboxamide;
(3S)-1-[3-[5-[[1-(Trifluoromethyl)cyclopropyl]methylamino]-2-pyridyl]azetidine-1-carbonyl]pyrrolidine-3-carboxamide;
(3S)-1-[3-[5-[[1-(Trifluoromethyl)cyclopropyl]methylamino]pyrazin-2-yl]azetidine-1-carbonyl]pyrrolidine-3-carboxamide;
(3S)-1-[3-[6-[3-(Trifluoromethyl)azetidin-1-yl]-3-pyridyl]azetidine-1-carbonyl]pyrrolidine-3-carboxamide;
(3S)-1-[3-[2-[3-(Trifluoromethyl)azetidin-1-yl]pyrimidin-5-yl]azetidine-1-carbonyl]pyrrolidine-3-carboxamide;
(3S)-1-[7-[(3,5-Difluoro-2-pyridyl)methyl]-2-azaspiro[3.5]nonane-2-carbonyl]pyrrolidine-3-carboxamide;
(3S)-1-[7-[(5-Chloro-2-pyridyl)methyl]-2-azaspiro[3.5]nonane-2-carbonyl]pyrrolidine-3-carboxamide;
(3S)-1-[7-[[5-(Trifluoromethyl)pyrazin-2-yl]methyl]-2-azaspiro[3.5]nonane-2-carbonyl]pyrrolidine-3-carboxamide;
(3S)-1-[7-[[5-(Trifluoromethyl)-2-pyridyl]methyl]-2-azaspiro[3.5]nonane-2-carbonyl]pyrrolidine-3-carboxamide;
(3S)-1-[7-[[6-(Difluoromethoxy)-3-pyridyl]methyl]-2-azaspiro[3.5]nonane-2-carbonyl]pyrrolidine-3-carboxamide;
(3S)-1-[6-[[6-(Trifluoromethyl)pyridazin-3-yl]methyl]-2-azaspiro[3.3]heptane-2-carbonyl]pyrrolidine-3-carboxamide;
(3S)-1-[6-[[2-(Trifluoromethyl)pyrimidin-5-yl]methyl]-2-azaspiro[3.3]heptane-2-carbonyl]pyrrolidine-3-carboxamide;
(3S)-1-[(6S)-6-[5-(Trifluoromethyl)pyrazin-2-yl]oxy-2-azaspiro[3.4]octane-2-carbonyl]pyrrolidine-3-carboxamide;
[6-(4-Fluorophenyl)-2-azaspiro[3.3]heptan-2-yl]-[(3S)-3-(tetrazol-1-yl)pyrrolidin-1-yl]methanone;
[(3S)-3-(Tetrazol-1-yl)pyrrolidin-1-yl]-[6-[[4-(trifluoromethylsulfonyl)phenyl]methyl]-2,6-diazaspiro[3.3]heptan-2-yl]methanone;
(3S)-1-[3-[4-(5-Cyclobutyl-4H-1,2,4-triazol-3-yl)phenyl]azetidine-1-carbonyl]pyrrolidine-3-carboxamide;
(3S)-1-[6-(3-Cyclopropyl-1,2,4-triazol-1-yl)-2-azaspiro[3.3]heptane-2-carbonyl]pyrrolidine-3-carboxamide;
[6-[(2,4-Difluorophenyl)methyl]-2-azaspiro[3.3]heptan-2-yl]-[(3S)-3-(triazol-1-yl)pyrrolidin-1-yl]methanone;
[3-[6-(2-Chlorophenoxy)-3-pyridyl]azetidin-1-yl]-[(3R)-3-(triazol-1-yl)pyrrolidin-1-yl]methanone;
[3-[4-(4-Chloro-2-methylsulfonyl-phenyl)phenyl]azetidin-1-yl]-[(3R)-3-(triazol-1-yl)pyrrolidin-1-yl]methanone;
[6-[(2,4-Difluorophenyl)methyl]-2-azaspiro[3.3]heptan-2-yl]-[(3R)-3-(triazol-1-yl)pyrrolidin-1-yl]methanone;
[3-[4-(4-Chloro-2-methylsulfonyl-phenyl)phenyl]azetidin-1-yl]-[(3R)-3-(triazol-2-yl)pyrrolidin-1-yl]methanone;
[6-[(2,4-Difluorophenyl)methyl]-2-azaspiro[3.3]heptan-2-yl]-[(3R)-3-(triazol-2-yl)pyrrolidin-1-yl]methanone;
[3-[[2-Fluoro-4-(trifluoromethyl)phenyl]methyl-methyl-amino]azetidin-1-yl]-[(3R)-3-(triazol-2-yl)pyrrolidin-1-yl]methanone;
[3-[4-(4-Chloro-2-methylsulfonyl-phenyl)phenyl]azetidin-1-yl]-[(3S)-3-(triazol-1-yl)pyrrolidin-1-yl]methanone;
[7-[[6-(Difluoromethoxy)-3-pyridyl]methyl]-2-azaspiro[3.5]nonan-2-yl]-[(3R)-3-(triazol-1-yl)pyrrolidin-1-yl]methanone;
[3-[4-(4-Chloro-2-methylsulfonyl-phenyl)phenyl]azetidin-1-yl]-[(3S)-3-(triazol-2-yl)pyrrolidin-1-yl]methanone;
[3-[4-(4-Chloro-2-methylsulfonyl-phenyl)phenyl]azetidin-1-yl]-[6-(triazol-2-yl)-2-azaspiro[3.3]heptan-2-yl]methanone;
[3-[4-(4-Chloro-2-methylsulfonyl-phenyl)phenyl]azetidin-1-yl]-[6-(triazol-1-yl)-2-azaspiro[3.3]heptan-2-yl]methanone;
(3S)-1-[3-[4-(3-Cyclopropyl-5-methyl-pyrazol-1-yl)phenyl]azetidine-1-carbonyl]pyrrolidine-3-carboxamide;
(3S)-1-[3-[4-[2-Methylsulfonyl-4-(trifluoromethyl)phenyl]phenyl]azetidine-1-carbonyl]pyrrolidine-3-carboxamide;
(3S)-1-[7-(4-Chloro-2-methylsulfonyl-phenyl)-2-azaspiro[3.5]nonane-2-carbonyl]pyrrolidine-3-carboxamide;
[3-[[2-Fluoro-4-(trifluoromethyl)phenyl]methoxy]azetidin-1-yl]-[(3S)-3-(1H-1,2,4-triazol-5-yl)pyrrolidin-1-yl]methanone;
(3S)-1-[3-[4-(5-Cyclopropyl-4H-1,2,4-triazol-3-yl)phenyl]azetidine-1-carbonyl]pyrrolidine-3-carboxamide;
(3S)-1-[3-[4-[5-[1-(Trifluoromethyl)cyclopropyl]-4H-1,2,4-triazol-3-yl]phenyl]azetidine-1-carbonyl]pyrrolidine-3-carboxamide;
(3S)-1-[6-[[5-(Trifluoromethyl)pyrimidin-2-yl]methyl]-2-azaspiro[3.3]heptane-2-carbonyl]pyrrolidine-3-carboxamide;
[(3S)-3-(1H-1,2,4-Triazol-5-yl)pyrrolidin-1-yl]-[6-[[5-(trifluoromethyl)pyrimidin-2-yl]methyl]-2-azaspiro[3.3]heptan-2-yl]methanone;
(3S)-1-[6-[[5-(Trifluoromethoxy)-2-pyridyl]methyl]-2-azaspiro[3.3]heptane-2-carbonyl]pyrrolidine-3-carboxamide;
[(3S)-3-(1H-1,2,4-Triazol-5-yl)pyrrolidin-1-yl]-[6-[[5-(trifluoromethoxy)-2-pyridyl]methyl]-2-azaspiro[3.3]heptan-2-yl]methanone;
rac-(3S)-1-[6-[[6-(Trifluoromethyl)-3-pyridyl]methyl]-2-azaspiro[3.4]octane-2-carbonyl]pyrrolidine-3-carboxamide;
[rac-(3S)-3-(1H-1,2,4-Triazol-5-yl)pyrrolidin-1-yl]-[6-[[6-(trifluoromethyl)-3-pyridyl]methyl]-2-azaspiro[3.4]octan-2-yl]methanone;
(3S)-1-[7-(4-Chloro-2-fluoro-phenyl)-2,7-diazaspiro[3.5]nonane-2-carbonyl]pyrrolidine-3-carboxamide;
(3S)-1-[7-(5-Chloro-3-fluoro-2-pyridyl)-2,7-diazaspiro[3.5]nonane-2-carbonyl]pyrrolidine-3-carboxamide;
1-[4-[1-[(3S)-3-(1H-1,2,4-Triazol-5-yl)pyrrolidine-1-carbonyl]azetidin-3-yl]phenyl]-4-(trifluoromethyl)pyridin-2-one;
[3-[4-(3-Cyclopropyl-5-methyl-pyrazol-1-yl)phenyl]azetidin-1-yl]-[(3S)-3-(1H-1,2,4-triazol-5-yl)pyrrolidin-1-yl]methanone;
[(3S)-3-(1H-1,2,4-Triazol-5-yl)pyrrolidin-1-yl]-[3-[6-[(3R)-3-(trifluoromethyl)pyrrolidin-1-yl]-3-pyridyl]azetidin-1-yl]methanone;
[(3S)-3-(1H-1,2,4-Triazol-5-yl)pyrrolidin-1-yl]-[3-[6-[(3S)-3-(trifluoromethyl)pyrrolidin-1-yl]-3-pyridyl]azetidin-1-yl]methanone;
[(3S)-3-(1H-1,2,4-Triazol-5-yl)pyrrolidin-1-yl]-[3-[6-[3-(trifluoromethyl)azetidin-1-yl]-3-pyridyl]azetidin-1-yl]methanone;
[(3S)-3-(1H-1,2,4-Triazol-5-yl)pyrrolidin-1-yl]-[3-[2-[3-(trifluoromethyl)azetidin-1-yl]pyrimidin-5-yl]azetidin-1-yl]methanone;
[3-[4-(2-Chloro-4-methylsulfonyl-phenyl)phenyl]azetidin-1-yl]-[(3S)-3-(1H-1,2,4-triazol-5-yl)pyrrolidin-1-yl]methanone;
[3-[5-(4-Chloro-2-methylsulfonyl-phenyl)-2-pyridyl]azetidin-1-yl]-[(3S)-3-(1H-1,2,4-triazol-5-yl)pyrrolidin-1-yl]methanone;
[7-[(5-Fluoro-2-pyridyl)methyl]-2-azaspiro[3.5]nonan-2-yl]-[(3S)-3-(1H-1,2,4-triazol-5-yl)pyrrolidin-1-yl]methanone;
[(3S)-3-(1H-1,2,4-Triazol-5-yl)pyrrolidin-1-yl]-[3-[5-[[1-(trifluoromethyl)cyclopropyl]methylamino]pyrazin-2-yl]azetidin-1-yl]methanone;
[7-[(5-Chloro-2-pyridyl)methyl]-2-azaspiro[3.5]nonan-2-yl]-[(3S)-3-(1H-1,2,4-triazol-5-yl)pyrrolidin-1-yl]methanone;
[(3S)-3-(1H-1,2,4-Triazol-5-yl)pyrrolidin-1-yl]-[7-[[5-(trifluoromethyl)pyrazin-2-yl]methyl]-2-azaspiro[3.5]nonan-2-yl]methanone;
[7-[[6-(Difluoromethoxy)-3-pyridyl]methyl]-2-azaspiro[3.5]nonan-2-yl]-[(3S)-3-(1H-1,2,4-triazol-5-yl)pyrrolidin-1-yl]methanone;
[(3S)-3-(1H-1,2,4-Triazol-5-yl)pyrrolidin-1-yl]-[7-[[1-(trifluoromethyl)cyclopropyl]methoxy]-2-azaspiro[3.5]nonan-2-yl]methanone;
[(3S)-3-(1H-1,2,4-Triazol-5-yl)pyrrolidin-1-yl]-[7-[[4-(trifluoromethylsulfonyl)phenyl]methyl]-2,7-diazaspiro[3.5]nonan-2-yl]methanone;
[(3S)-3-(1H-1,2,4-Triazol-5-yl)pyrrolidin-1-yl]-[7-[[3-(trifluoromethylsulfonyl)phenyl]methyl]-2,7-diazaspiro[3.5]nonan-2-yl]methanone;
[6-[[3-Fluoro-5-(trifluoromethyl)-2-pyridyl]methyl]-2-azaspiro[3.3]heptan-2-yl]-[(3S)-3-(1H-1,2,4-triazol-5-yl)pyrrolidin-1-yl]methanone;
[(3S)-3-(1H-1,2,4-Triazol-5-yl)pyrrolidin-1-yl]-[6-[[6-(trifluoromethyl)pyridazin-3-yl]methyl]-2-azaspiro[3.3]heptan-2-yl]methanone;
[(3S)-3-(1H-1,2,4-Triazol-5-yl)pyrrolidin-1-yl]-[6-[[2-(trifluoromethyl)pyrimidin-5-yl]methyl]-2-azaspiro[3.3]heptan-2-yl]methanone;
[6-[[4-Fluoro-2-(trifluoromethyl)phenyl]methyl]-2-azaspiro[3.3]heptan-2-yl]-[(3S)-3-(1H-1,2,4-triazol-5-yl)pyrrolidin-1-yl]methanone;
[6-[[2-Fluoro-4-(trifluoromethyl)phenyl]methyl]-2-azaspiro[3.3]heptan-2-yl]-[(3S)-3-(1H-1,2,4-triazol-5-yl)pyrrolidin-1-yl]methanone;
[6-[[3-Fluoro-5-(trifluoromethyl)phenyl]methyl]-2-azaspiro[3.3]heptan-2-yl]-[(3S)-3-(1H-1,2,4-triazol-5-yl)pyrrolidin-1-yl]methanone;
[(3S)-3-(1H-1,2,4-Triazol-5-yl)pyrrolidin-1-yl]-[(6S)-6-[[5-(trifluoromethyl)pyrazin-2-yl]methyl]-2-azaspiro[3.4]octan-2-yl]methanone;
[(3S)-3-(1H-1,2,4-Triazol-5-yl)pyrrolidin-1-yl]-[(6R)-6-[[5-(trifluoromethyl)pyrazin-2-yl]methyl]-2-azaspiro[3.4]octan-2-yl]methanone;
[6-[(4-Methylsulfonylphenyl)methyl]-2-azaspiro[3.4]octan-2-yl]-[rac-(3S)-3-(1H-1,2,4-triazol-5-yl)pyrrolidin-1-yl]methanone;
[(3S)-3-(1H-1,2,4-Triazol-5-yl)pyrrolidin-1-yl]-[(6S)-6-[5-(trifluoromethyl)pyrazin-2-yl]oxy-2-azaspiro[3.4]octan-2-yl]methanone;
[(3S)-3-(1H-1,2,4-Triazol-5-yl)pyrrolidin-1-yl]-[3-[1-[[4-(trifluoromethyl)phenyl]methyl]azetidin-3-yl]azetidin-1-yl]methanone;
[(3S)-3-(1H-1,2,4-Triazol-5-yl)pyrrolidin-1-yl]-[6-[[5-(trifluoromethyl)-3-pyridyl]methyl]-2-azaspiro[3.3]heptan-2-yl]methanone;
[(3S)-3-(1H-1,2,4-Triazol-5-yl)pyrrolidin-1-yl]-[6-[[4-(trifluoromethyl)-2-pyridyl]methyl]-2-azaspiro[3.3]heptan-2-yl]methanone;
[(3S)-3-(1H-1,2,4-Triazol-5-yl)pyrrolidin-1-yl]-[7-[5-(trifluoromethyl)-2-pyridyl]methyl]-2,7-diazaspiro[3.4]octan-2-yl]methanone;
[(3S)-3-(1H-1,2,4-Triazol-5-yl)pyrrolidin-1-yl]-[3-[3-[1-(trifluoromethyl)cyclopropyl]methylamino]-1-bicyclo[1.1.1]pentanyl]azetidin-1-yl]methanone;
[(3S)-3-(1H-1,2,4-Triazol-5-yl)pyrrolidin-1-yl]-[3-[3-[1-(trifluoromethyl)cyclopropyl]amino]methyl]-1-bicyclo[1.1.1]pentanyl]azetidin-1-yl]methanone;
[3-[3-(5-Cyclopropyl-1,3,4-oxadiazol-2-yl)-1-bicyclo[1.1.1]pentanyl]azetidin-1-yl]-[(3S)-3-(1H-1,2,4-triazol-5-yl)pyrrolidin-1-yl]methanone;
[(3S)-3-(1H-1,2,4-Triazol-5-yl)pyrrolidin-1-yl]-[3-[3-[5-(2,2,2-trifluoroethyl)-1,3,4-oxadiazol-2-yl]-1-bicyclo[1.1.1]pentanyl]azetidin-1-yl]methanone;
(4S)-1-[4-[1-[(3S)-3-(1H-1,2,4-Triazol-5-yl)pyrrolidine-1-carbonyl]azetidin-3-yl]phenyl]-4-(trifluoromethyl) piperidin-2-one;
(4R)-1-[4-[1-[(3S)-3-(1H-1,2,4-Triazol-5-yl)pyrrolidine-1-carbonyl]azetidin-3-yl]phenyl]-4-(trifluoromethyl) piperidin-2-one;
[3-[4-[3-(Difluoromethyl)-5-methyl-pyrazol-1-yl]phenyl]azetidin-1-yl]-[(3S)-3-(1H-1,2,4-triazol-5-yl)pyrrolidin-1-yl]methanone;
[3-[4-[5-Methyl-3-(trifluoromethyl)pyrazol-1-yl]phenyl]azetidin-1-yl]-[(3S)-3-(1H-1,2,4-triazol-5-yl)pyrrolidin-1-yl]methanone;
[3-[4-(3-tert-Butyl-5-methyl-pyrazol-1-yl)phenyl]azetidin-1-yl]-[(3S)-3-(1H-1,2,4-triazol-5-yl)pyrrolidin-1-yl]methanone;
(3S)-1-[6-[[3-Fluoro-5-(trifluoromethyl)-2-pyridyl]methyl]-2-azaspiro[3.3]heptane-2-carbonyl]pyrrolidine-3-carboxamide;
(3S)-1-[7-[5-(Trifluoromethyl)-2-pyridyl]methyl]-2,7-diazaspiro[3.4]octane-2-carbonyl]pyrrolidine-3-carboxamide;
(3S)-1-[3-[3-[5-(2,2,2-Trifluoroethyl)-1,3,4-oxadiazol-2-yl]-1-bicyclo[1.1.1]pentanyl]azetidine-1-carbonyl]pyrrolidine-3-carboxamide;
[6-[[4-Methylsulfonyl-3-(trifluoromethyl)phenyl]methyl]-2-azaspiro[3.3]heptan-2-yl]-[(3S)-3-(1H-1,2,4-triazol-5-yl)pyrrolidin-1-yl]methanone;
[6-[2-Methylsulfonyl-4-(trifluoromethyl)phenyl]methyl]-2-azaspiro[3.3]heptan-2-yl]-[(3S)-3-(1H-1,2,4-triazol-5-yl)pyrrolidin-1-yl]methanone;
[6-[3-Methylsulfonyl-4-(trifluoromethyl)phenyl]methyl]-2-azaspiro[3.3]heptan-2-yl]-[(3S)-3-(1H-1,2,4-triazol-5-yl)pyrrolidin-1-yl]methanone;
[rac-(3S)-3-(1H-1,2,4-Triazol-5-yl)pyrrolidin-1-yl]-[6-[3-(trifluoromethylsulfonimidoyl)phenyl]methyl]-2-azaspiro[3.3]heptan-2-yl]methanone;
[rac-(3S)-3-(1H-1,2,4-Triazol-5-yl)pyrrolidin-1-yl]-[6-[[4-(trifluoromethylsulfonimidoyl)phenyl]methyl]-2-azaspiro[3.3]heptan-2-yl]methanone;
[(3S)-3-(1H-1,2,4-Triazol-5-yl)pyrrolidin-1-yl]-[6-[4-(trifluoromethyl)pyrazol-1-yl]methyl]-2-azaspiro[3.3]heptan-2-yl]methanone;
[(3S)-3-(1H-1,2,4-Triazol-5-yl)pyrrolidin-1-yl]-[3-[4-[5-[1-(trifluoromethyl)cyclopropyl]-4H-1,2,4-triazol-3-yl]phenyl]azetidin-1-yl]methanone;
(3S)-1-[3-[3-[[1-(Trifluoromethyl)cyclopropyl]methylamino]-1-bicyclo[1.1.1]pentanyl]azetidine-1-carbonyl]pyrrolidine-3-carboxamide;
(3S)-1-[3-[3-[[1-(Trifluoromethyl)cyclopropyl]amino]methyl]-1-bicyclo[1.1.1]pentanyl]azetidine-1-carbonyl]pyrrolidine-3-carboxamide;
rac-(1S,4R,5R)-2-[3-[4-(4-Chloro-2-methylsulfonyl-phenyl)phenyl]azetidine-1-carbonyl]-2-azabicyclo[2.1.1]hexane-5-carboxamide;
(3S)-1-[3-[3-(5-Cyclopropyl-1,3,4-oxadiazol-2-yl)-1-bicyclo[1.1.1]pentanyl]azetidine-1-carbonyl]pyrrolidine-3-carboxamide;
(3S)-1-[7-(4-Chloro-2-methylsulfonyl-phenyl)-2,7-diazaspiro[3.5]nonane-2-carbonyl]pyrrolidine-3-carboxamide;
[6-[(5-Methylsulfonyl-2-pyridyl)methyl]-2-azaspiro[3.3]heptan-2-yl]-[(3S)-3-(1H-1,2,4-triazol-5-yl)pyrrolidin-1-yl]methanone;
[3-[4-[3-(2,2-Dimethylpropyl)triazol-4-yl]phenyl]azetidin-1-yl]-[(3S)-3-(1H-1,2,4-triazol-5-yl)pyrrolidin-1-yl]methanone;
[(3S)-3-(1H-1,2,4-Triazol-5-yl)pyrrolidin-1-yl]-[6-[[3-(trifluoromethyl)-1,2,4-triazol-1-yl]methyl]-2-azaspiro[3.3]heptan-2-yl]methanone;
[(3S)-3-(1H-1,2,4-Triazol-5-yl)pyrrolidin-1-yl]-[6-[4-(trifluoromethyl) oxazol-2-yl]methyl]-2-azaspiro[3.3]heptan-2-yl]methanone;
[(3S)-3-(1H-1,2,4-Triazol-5-yl)pyrrolidin-1-yl]-[6-[3-(trifluoromethyl)-1,2,4-thiadiazol-5-yl]methyl]-2-azaspiro[3.3]heptan-2-yl]methanone;
[(3S)-3-(1H-1,2,4-Triazol-5-yl)pyrrolidin-1-yl]-[3-[3-[5-(trifluoromethyl)pyrazin-2-yl]oxy-1-bicyclo[1.1.1]pentanyl]azetidin-1-yl]methanone;
[(3S)-3-(1H-1,2,4-Triazol-5-yl)pyrrolidin-1-yl]-[6-[[4-(trifluoromethyl) imidazol-1-yl]methyl]-2-azaspiro[3.3]heptan-2-yl]methanone;
[3-[4-(3,5-Diisopropylpyrazol-1-yl)phenyl]azetidin-1-yl]-[(3S)-3-(1H-1,2,4-triazol-5-yl)pyrrolidin-1-yl]methanone;
[3-[4-(3,5-Dimethyl-1,2,4-triazol-1-yl)phenyl]azetidin-1-yl]-[(3S)-3-(1H-1,2,4-triazol-5-yl)pyrrolidin-1-yl]methanone;
[(3S)-3-(4H-1,2,4-Triazol-3-yl)pyrrolidin-1-yl]-[7-[5-(trifluoromethyl)-2-pyridyl]amino]-2-azaspiro[3.5]nonan-2-yl]methanone;
[6-[(3-Fluoro-5-methylsulfonyl-phenyl)methyl]-2-azaspiro[3.3]heptan-2-yl]-[(3S)-3-(4H-1,2,4-triazol-3-yl)pyrrolidin-1-yl]methanone;
[6-[[4-Methylsulfonyl-3-(trifluoromethyl)phenyl]methyl]-2-azaspiro[3.4]octan-2-yl]-[rac-(3S)-3-(4H-1,2,4-triazol-3-yl)pyrrolidin-1-yl]methanone;
[3-[[2-Fluoro-4-(trifluoromethylsulfonyl)phenyl]methoxy]azetidin-1-yl]-[(3S)-3-(4H-1,2,4-triazol-3-yl)pyrrolidin-1-yl]methanone;
[(3S)-3-(4H-1,2,4-Triazol-3-yl)pyrrolidin-1-yl]-[7-[6-(trifluoromethyl)-3-pyridyl]methyl]-2-azaspiro[3.5]nonan-2-yl]methanone;
[3-[6-(2-Chlorophenoxy)-3-pyridyl]azetidin-1-yl]-[(3S)-3-(4H-1,2,4-triazol-3-yl)pyrrolidin-1-yl]methanone;
[6-[(3,5-Difluoro-2-pyridyl)methyl]-2-azaspiro[3.3]heptan-2-yl]-[(3S)-3-(4H-1,2,4-triazol-3-yl)pyrrolidin-1-yl]methanone;
[7-[Methyl-[5-(trifluoromethyl)pyrazin-2-yl]amino]-2-azaspiro[3.5]nonan-2-yl]-[(3S)-3-(4H-1,2,4-triazol-3-yl)pyrrolidin-1-yl]methanone;
[6-[2-Methylsulfonyl-4-(trifluoromethyl)phenyl]-2-azaspiro[3.3]heptan-2-yl]-[(3S)-3-(4H-1,2,4-triazol-3-yl)pyrrolidin-1-yl]methanone;
[(3S)-3-(4H-1,2,4-Triazol-3-yl)pyrrolidin-1-yl]-[3-[[4-(trifluoromethylsulfonyl)phenyl]methoxy]azetidin-1-yl]methanone;
[6-(4-Methylsulfonylphenyl)-2-azaspiro[3.3]heptan-2-yl]-[(3S)-3-(4H-1,2,4-triazol-3-yl)pyrrolidin-1-yl]methanone;
[(3S)-3-(4H-1,2,4-Triazol-3-yl)pyrrolidin-1-yl]-[6-[4-(trifluoromethylsulfonyl)phenyl]-2-azaspiro[3.3]heptan-2-yl]methanone;
[(3S)-3-(4H-1,2,4-Triazol-3-yl)pyrrolidin-1-yl]-[3-[4-[2-(trifluoromethyl)pyrimidin-5-yl]oxyphenyl]azetidin-1-yl]methanone;
[(3S)-3-(4H-1,2,4-Triazol-3-yl)pyrrolidin-1-yl]-[7-[[5-(trifluoromethyl)pyrazin-2-yl]amino]-2-azaspiro[3.5]nonan-2-yl]methanone;
[7-(5-Chloro-2-methylsulfonyl-phenoxy)-2-azaspiro[3.5]nonan-2-yl]-[(3S)-3-(4H-1,2,4-triazol-3-yl)pyrrolidin-1-yl]methanone;
[3-[4-(3,5-Dimethylpyrazol-1-yl)phenyl]azetidin-1-yl]-[(3S)-3-(4H-1,2,4-triazol-3-yl)pyrrolidin-1-yl]methanone;
[3-[6-(N-Methylanilino)-3-pyridyl]azetidin-1-yl]-[(3S)-3-(4H-1,2,4-triazol-3-yl)pyrrolidin-1-yl]methanone;
2-Methoxy-4-[[2-[(3S)-3-(4H-1,2,4-triazol-3-yl)pyrrolidine-1-carbonyl]-2-azaspiro[3.3]heptan-6-yl]methyl]benzonitrile;
[3-[4-[1-Methyl-3-(trifluoromethyl)pyrazol-4-yl]phenyl]azetidin-1-yl]-[(3S)-3-(4H-1,2,4-triazol-3-yl)pyrrolidin-1-yl]methanone;
[3-[6-(4-Fluorophenoxy)-3-pyridyl]azetidin-1-yl]-[(3S)-3-(4H-1,2,4-triazol-3-yl)pyrrolidin-1-yl]methanone;
[3-(4-Cyclopropyl-2-fluoro-phenoxy)azetidin-1-yl]-[(3S)-3-(4H-1,2,4-triazol-3-yl)pyrrolidin-1-yl]methanone;
[3-(4-Chloro-3-cyclopropyl-phenoxy)azetidin-1-yl]-[(3S)-3-(4H-1,2,4-triazol-3-yl)pyrrolidin-1-yl]methanone;
[3-[4-[5-[(1-Methylcyclopropyl)methyl]-4H-1,2,4-triazol-3-yl]phenyl]azetidin-1-yl]-[(3S)-3-(4H-1,2,4-triazol-3-yl)pyrrolidin-1-yl]methanone;
[(3S)-3-(1H-1,2,4-Triazol-5-yl)pyrrolidin-1-yl]-[6-[4-(trifluoromethyl) thiazol-2-yl]methyl]-2-azaspiro[3.3]heptan-2-yl]methanone;
(3S)-1-[6-[4-(Trifluoromethyl)pyrazol-1-yl]methyl]-2-azaspiro[3.3]heptane-2-carbonyl]pyrrolidine-3-carboxamide;
(3S)-1-[6-[[4-(Trifluoromethyl) imidazol-1-yl]methyl]-2-azaspiro[3.3]heptane-2-carbonyl]pyrrolidine-3-carboxamide;
(3S)-1-[6-[3-(Trifluoromethyl)-1,2,4-triazol-1-yl]methyl]-2-azaspiro[3.3]heptane-2-carbonyl]pyrrolidine-3-carboxamide;
(3S)-1-[6-[4-(Trifluoromethyl) oxazol-2-yl]methyl]-2-azaspiro[3.3]heptane-2-carbonyl]pyrrolidine-3-carboxamide;
(3S)-1-[6-[3-(Trifluoromethyl)-1,2,4-thiadiazol-5-yl]methyl]-2-azaspiro[3.3]heptane-2-carbonyl]pyrrolidine-3-carboxamide;
(3S)-1-[6-[4-(Trifluoromethyl) thiazol-2-yl]methyl]-2-azaspiro[3.3]heptane-2-carbonyl]pyrrolidine-3-carboxamide;
[6-[(5-Fluoro-3-pyridyl)methyl]-2-azaspiro[3.3]heptan-2-yl]-[(3S)-3-(4H-1,2,4-triazol-3-yl)pyrrolidin-1-yl]methanone;
[6-[(2-Fluoro-4-methylsulfonyl-phenyl)methyl]-2-azaspiro[3.3]heptan-2-yl]-[(3S)-3-(4H-1,2,4-triazol-3-yl)pyrrolidin-1-yl]methanone;
[6-([1,2,4]Triazolo[1,5-a]pyridin-7-ylmethyl)-2-azaspiro[3.3]heptan-2-yl]-[(3S)-3-(1H-1,2,4-triazol-5-yl)pyrrolidin-1-yl]methanone;
(3S)-1-[6-([1,2,4]Triazolo[1,5-a]pyridin-6-ylmethyl)-2-azaspiro[3.3]heptane-2-carbonyl]pyrrolidine-3-carboxamide;
[6-(1H-Pyrazolo[4,3-b]pyridin-5-ylmethyl)-2-azaspiro[3.3]heptan-2-yl]-[(3S)-3-(1H-1,2,4-triazol-5-yl)pyrrolidin-1-yl]methanone;
[(3S)-3-(1H-1,2,4-Triazol-5-yl)pyrrolidin-1-yl]-[6-[1-(trifluoromethyl)pyrazol-4-yl]methyl]-2-azaspiro[3.3]heptan-2-yl]methanone;
3-[[2-[(3S)-3-(1H-1,2,4-Triazol-5-yl)pyrrolidine-1-carbonyl]-2-azaspiro[3.3]heptan-6-yl]methyl]-5-(trifluoromethyl)benzonitrile;
4-[2-[(3S)-3-(1H-1,2,4-Triazol-5-yl)pyrrolidine-1-carbonyl]-2-azaspiro[3.3]heptan-6-yl]methyl]-2-(trifluoromethyl)benzonitrile;
[(3S)-3-(1H-1,2,4-Triazol-5-yl)pyrrolidin-1-yl]-[6-[6-(trifluoromethoxy)-3-pyridyl]methyl]-2-azaspiro[3.3]heptan-2-yl]methanone;
[(3S)-3-(1H-1,2,4-Triazol-5-yl)pyrrolidin-1-yl]-[6-[3-(trifluoromethylsulfonyl)phenyl]methyl]-2-azaspiro[3.3]heptan-2-yl]methanone;
[6-[(4-Cyclopropylsulfonylphenyl)methyl]-2-azaspiro[3.3]heptan-2-yl]-[(3S)-3-(1H-1,2,4-triazol-5-yl)pyrrolidin-1-yl]methanone;
[(3S)-3-(1H-1,2,4-Triazol-5-yl)pyrrolidin-1-yl]-[3-[3-[6-(trifluoromethyl)-3-pyridyl]methyl]-1-bicyclo[1.1.1]pentanyl]azetidin-1-yl]methanone;
[6-[[2-Fluoro-4-(trifluoromethyl)phenyl]methyl]-2-azaspiro[3.3]heptan-2-yl]-[3-(1H-1,2,4-triazol-5-yl)azetidin-1-yl]methanone;
[6-[(2,4-Difluorophenyl)methyl]-2-azaspiro[3.3]heptan-2-yl]-[3-(1H-1,2,4-triazol-5-yl)azetidin-1-yl]methanone;
[6-[4-Fluoro-2-(trifluoromethyl)phenyl]methyl]-2-azaspiro[3.3]heptan-2-yl]-[3-(1H-1,2,4-triazol-5-yl)azetidin-1-yl]methanone;
[6-[[3-Fluoro-5-(trifluoromethyl)phenyl]methyl]-2-azaspiro[3.3]heptan-2-yl]-[3-(1H-1,2,4-triazol-5-yl)azetidin-1-yl]methanone;
[6-[(3-Chloro-5-fluoro-2-pyridyl)methyl]-2-azaspiro[3.3]heptan-2-yl]-[3-(1H-1,2,4-triazol-5-yl)azetidin-1-yl]methanone;
[3-(1H-1,2,4-Triazol-5-yl)azetidin-1-yl]-[6-[[5-(trifluoromethoxy)-2-pyridyl]methyl]-2-azaspiro[3.3]heptan-2-yl]methanone;
[6-[[3-Fluoro-5-(trifluoromethyl)-2-pyridyl]methyl]-2-azaspiro[3.3]heptan-2-yl]-[3-(1H-1,2,4-triazol-5-yl)azetidin-1-yl]methanone;
[3-(1H-1,2,4-Triazol-5-yl)azetidin-1-yl]-[7-[5-(trifluoromethyl)-2-pyridyl]methyl]-2,7-diazaspiro[3.4]octan-2-yl]methanone;
[7-[(5-Chloro-2-pyridyl)methyl]-2-azaspiro[3.5]nonan-2-yl]-[3-(1H-1,2,4-triazol-5-yl)azetidin-1-yl]methanone;
[7-[6-(Difluoromethoxy)-3-pyridyl]methyl]-2-azaspiro[3.5]nonan-2-yl]-[3-(1H-1,2,4-triazol-5-yl)azetidin-1-yl]methanone;
[3-(1H-1,2,4-Triazol-5-yl)azetidin-1-yl]-[3-[4-[1-(trifluoromethyl)cyclopropyl]phenyl]azetidin-1-yl]methanone;
[3-(1H-1,2,4-Triazol-5-yl)azetidin-1-yl]-[3-[4-[3-(trifluoromethyl)azetidin-1-yl]phenyl]azetidin-1-yl]methanone;
[3-[4-(4-Chloro-2-methylsulfonyl-phenyl)phenyl]azetidin-1-yl]-[3-(1H-1,2,4-triazol-5-yl)azetidin-1-yl]methanone;
[3-[4-[5-[(1-Methylcyclopropyl)methyl]-4H-1,2,4-triazol-3-yl]phenyl]azetidin-1-yl]-[(3S)-3-(1H-1,2,4-triazol-5-yl)pyrrolidin-1-yl]methanone;
[3-[4-(4-Chloro-2-methylsulfonyl-phenyl)phenyl]azetidin-1-yl]-[3-(5-cyclopropyl-1H-1,2,4-triazol-3-yl)pyrrolidin-1-yl]methanone;
(3S)-1-[3-[4-[5-Methyl-3-(trifluoromethyl)pyrazol-1-yl]phenyl]azetidine-1-carbonyl]pyrrolidine-3-carboxamide;
[4-[(2-Chloro-4-methylsulfonyl-phenyl)methoxy]-1-piperidyl]-[(3S)-3-(1H-1,2,4-triazol-5-yl)pyrrolidin-1-yl]methanone;
(3S)-1-[4-[(2-Chloro-4-methylsulfonyl-phenyl)methoxy]piperidine-1-carbonyl]pyrrolidine-3-carboxamide;
[4-[4-Methylsulfonyl-3-(trifluoromethyl) phenoxy]methyl]-1-piperidyl]-[(3S)-3-(1H-1,2,4-triazol-5-yl)pyrrolidin-1-yl]methanone;
(3S)-1-[4-[[4-Methylsulfonyl-3-(trifluoromethyl) phenoxy]methyl]piperidine-1-carbonyl]pyrrolidine-3-carboxamide;
[3-[3-(5-Cyclopropyl-3-methyl-pyrazol-1-yl)-1-bicyclo[1.1.1]pentanyl]azetidin-1-yl]-[(3S)-3-(1H-1,2,4-triazol-5-yl)pyrrolidin-1-yl]methanone;
[3-[3-(3,5-Dimethylpyrazol-1-yl)-1-bicyclo[1.1.1]pentanyl]azetidin-1-yl]-[(3S)-3-(1H-1,2,4-triazol-5-yl)pyrrolidin-1-yl]methanone;
[4-[[2-Fluoro-4-(trifluoromethoxy)phenyl]methoxy]-1-piperidyl]-[(3S)-3-(1H-1,2,4-triazol-5-yl)pyrrolidin-1-yl]methanone;
[4-[[2-Fluoro-4-(trifluoromethyl)phenyl]methoxy]-1-piperidyl]-[(3S)-3-(1H-1,2,4-triazol-5-yl)pyrrolidin-1-yl]methanone;
(3S)-1-[4-[[2-Fluoro-4-(trifluoromethoxy)phenyl]methoxy]piperidine-1-carbonyl]pyrrolidine-3-carboxamide;
(3S)-1-[4-[2-Fluoro-4-(trifluoromethyl)phenyl]methoxy]piperidine-1-carbonyl]pyrrolidine-3-carboxamide;
Methyl 5-[2-[(3S)-3-(1H-1,2,4-triazol-5-yl)pyrrolidine-1-carbonyl]-2-azaspiro[3.3]heptan-6-yl]methyl]-2-(trifluoromethoxy)benzoate;
[4-[2-Methylsulfonyl-4-(trifluoromethyl) phenoxy]methyl]-1-piperidyl]-[(3S)-3-(1H-1,2,4-triazol-5-yl)pyrrolidin-1-yl]methanone;
[3-[4-(4-Chloro-2-methylsulfonyl-phenyl)phenyl]azetidin-1-yl]-[4-(5-cyclopropyl-1H-1,2,4-triazol-3-yl) piperazin-1-yl]methanone;
[(3S)-3-(1H-1,2,4-Triazol-5-yl)pyrrolidin-1-yl]-[6-[5-(trifluoromethylsulfonyl)-2-pyridyl]methyl]-2-azaspiro[3.3]heptan-2-yl]methanone;
[4-(5-Chlorooxazolo[4,5-b]pyridin-2-yl) piperazin-1-yl]-[3-[[2-fluoro-4-(trifluoromethoxy)phenyl]methoxy]azetidin-1-yl]methanone;
4-[[2-[(3S)-3-(1H-1,2,4-Triazol-5-yl)pyrrolidine-1-carbonyl]-2-azaspiro[3.3]heptan-6-yl]methyl]benzenesulfonamide;
[(3S)-3-(1H-1,2,4-Triazol-5-yl)pyrrolidin-1-yl]-[6-[5-[3-(trifluoromethyl)azetidin-1-yl]pyrazin-2-yl]methyl]-2-azaspiro[3.3]heptan-2-yl]methanone;
[(3S)-3-(4H-1,2,4-Triazol-3-yl)pyrrolidin-1-yl]-[6-[5-[1-(trifluoromethyl)cyclopropyl]methylamino]pyrazin-2-yl]methyl]-2-azaspiro[3.3]heptan-2-yl]methanone;
[(3S)-3-(4H-1,2,4-Triazol-3-yl)pyrrolidin-1-yl]-[6-[4-[3-(trifluoromethyl)azetidin-1-yl]sulfonylphenyl]methyl]-2-azaspiro[3.3]heptan-2-yl]methanone;
[(3S)-3-(1H-1,2,4-Triazol-5-yl)pyrrolidin-1-yl]-[(6S)-6-[[4-(trifluoromethylsulfonyl)phenyl]methyl]-2-azaspiro[3.4]octan-2-yl]methanone;
[(3S)-3-(1H-1,2,4-Triazol-5-yl)pyrrolidin-1-yl]-[(6R)-6-[[4-(trifluoromethylsulfonyl)phenyl]methyl]-2-azaspiro[3.4]octan-2-yl]methanone;
[(3S)-3-(1H-1,2,4-Triazol-5-yl)pyrrolidin-1-yl]-[(6S)-6-[3-(trifluoromethylsulfonyl)phenyl]methyl]-2-azaspiro[3.4]octan-2-yl]methanone;
[(3S)-3-(1H-1,2,4-Triazol-5-yl)pyrrolidin-1-yl]-[(6R)-6-[3-(trifluoromethylsulfonyl)phenyl]methyl]-2-azaspiro[3.4]octan-2-yl]methanone;
[6-[1-Methyl-3-(trifluoromethyl)pyrazol-4-yl]methyl]-2-azaspiro[3.3]heptan-2-yl]-[(3S)-3-(1H-1,2,4-triazol-5-yl)pyrrolidin-1-yl]methanone;
[6-[2-Methyl-5-(trifluoromethyl)pyrazol-3-yl]methyl]-2-azaspiro[3.3]heptan-2-yl]-[(3S)-3-(1H-1,2,4-triazol-5-yl)pyrrolidin-1-yl]methanone;
[6-[1-Methyl-5-(trifluoromethyl)pyrazol-4-yl]methyl]-2-azaspiro[3.3]heptan-2-yl]-[(3S)-3-(1H-1,2,4-triazol-5-yl)pyrrolidin-1-yl]methanone;
[6-[2-Methyl-4-(trifluoromethyl)pyrazol-3-yl]methyl]-2-azaspiro[3.3]heptan-2-yl]-[(3S)-3-(1H-1,2,4-triazol-5-yl)pyrrolidin-1-yl]methanone;
[(3S)-3-(1H-1,2,4-Triazol-5-yl)pyrrolidin-1-yl]-[6-[[1-(2,2,2-trifluoroethyl)pyrazol-4-yl]methyl]-2-azaspiro[3.3]heptan-2-yl]methanone;
4-[[2-[(3S)-3-(1H-1,2,4-Triazol-5-yl)pyrrolidine-1-carbonyl]-2-azaspiro[3.3]heptan-6-yl]methyl]-2-(trifluoromethoxy)benzamide;
[(3S)-3-(4H-1,2,4-Triazol-3-yl)pyrrolidin-1-yl]-[3-[5-[1-(trifluoromethyl)cyclopropyl]-2-pyridyl]azetidin-1-yl]methanone;
[(3S)-3-(4H-1,2,4-Triazol-3-yl)pyrrolidin-1-yl]-[3-[[6-(trifluoromethyl)-3-pyridyl]methoxymethyl]azetidin-1-yl]methanone;
[3-[4-(1-Methylsulfonylcyclopropyl)phenyl]azetidin-1-yl]-[(3S)-3-(1H-1,2,4-triazol-5-yl)pyrrolidin-1-yl]methanone;
[4-[(3-Chloro-5-methylsulfonyl-phenoxy)methyl]-1-piperidyl]-[(3S)-3-(1H-1,2,4-triazol-5-yl)pyrrolidin-1-yl]methanone;
5-[2-[(3S)-3-(1H-1,2,4-Triazol-5-yl)pyrrolidine-1-carbonyl]-2-azaspiro[3.3]heptan-6-yl]methyl]-2-(trifluoromethoxy)benzamide;
[rac-(3S)-3-(1H-1,2,4-Triazol-5-yl)pyrrolidin-1-yl]-[3-[4-(trifluoromethylsulfonimidoyl)phenyl]azetidin-1-yl]methanone;
[(3S)-3-(1H-1,2,4-Triazol-5-yl)pyrrolidin-1-yl]-[3-[3-[[5-(trifluoromethyl)pyrazin-2-yl]amino]-1-bicyclo[1.1.1]pentanyl]azetidin-1-yl]methanone;
Methyl 4-[[2-[(3S)-3-(1H-1,2,4-triazol-5-yl)pyrrolidine-1-carbonyl]-2-azaspiro[3.3]heptan-6-yl]methyl]-2-(trifluoromethoxy)benzoate;
[4-[2-Methylsulfonyl-4-(trifluoromethoxy) phenoxy]methyl]-1-piperidyl]-[(3S)-3-(4H-1,2,4-triazol-3-yl)pyrrolidin-1-yl]methanone;
[(3S)-3-(4H-1,2,4-Triazol-3-yl)pyrrolidin-1-yl]-[4-[6-(trifluoromethyl)-3-pyridyl]methoxymethyl]-1-piperidyl]methanone;
[(3S)-3-(4H-1,2,4-Triazol-3-yl)pyrrolidin-1-yl]-[4-[6-(trifluoromethyl)-3-pyridyl]oxymethyl]-1-piperidyl]methanone;
[(3S)-3-(4H-1,2,4-Triazol-3-yl)pyrrolidin-1-yl]-[4-[6-(trifluoromethyl)-3-pyridyl]methoxy]-1-piperidyl]methanone;
[4-[[4-Fluoro-3-(trifluoromethyl) phenoxy]methyl]-1-piperidyl]-[(3S)-3-(4H-1,2,4-triazol-3-yl)pyrrolidin-1-yl]methanone;
[4-[[3-Fluoro-5-(trifluoromethyl) phenoxy]methyl]-1-piperidyl]-[(3S)-3-(4H-1,2,4-triazol-3-yl)pyrrolidin-1-yl]methanone;
[3-(3-Isobutyl-1H-pyrazol-5-yl)pyrrolidin-1-yl]-[6-[5-(trifluoromethyl)pyrazin-2-yl]methyl]-2-azaspiro[3.3]heptan-2-yl]methanone;
[(3S)-3-(4H-1,2,4-Triazol-3-yl)pyrrolidin-1-yl]-[6-[5-(trifluoromethylsulfonyl)-3-pyridyl]methyl]-2-azaspiro[3.3]heptan-2-yl]methanone;
[6-[[3-Fluoro-5-(trifluoromethylsulfonyl)phenyl]methyl]-2-azaspiro[3.3]heptan-2-yl]-[(3S)-3-(4H-1,2,4-triazol-3-yl)pyrrolidin-1-yl]methanone;
N′-[2-[3-[1-[(3S)-3-(4H-1,2,4-Triazol-3-yl)pyrrolidine-1-carbonyl]azetidin-3-yl]-1-bicyclo[1.1.1]pentanyl]acetyl]cyclopropanecarbohydrazide;
[4-[2-Methylsulfonyl-4-(trifluoromethoxy)phenyl]methoxy]-1-piperidyl]-[(3S)-3-(1H-1,2,4-triazol-5-yl)pyrrolidin-1-yl]methanone;
(3S)-1-[4-[2-Methylsulfonyl-4-(trifluoromethoxy)phenyl]methoxy]piperidine-1-carbonyl]pyrrolidine-3-carboxamide;
(3S)-1-[3-[3-(4-Chloro-2-methylsulfonyl-phenyl)-1-bicyclo[1.1.1]pentanyl]azetidine-1-carbonyl]pyrrolidine-3-carboxamide;
2-Methoxy-4-[1-[(3S)-3-(1H-1,2,4-triazol-5-yl)pyrrolidine-1-carbonyl]-4-piperidyl]oxymethyl]benzonitrile;
(3S)-1-[2-(4-Chloro-2-methylsulfonyl-phenyl)-6-azaspiro[3.4]octane-6-carbonyl]pyrrolidine-3-carboxamide;
2-Methoxy-4-[1-[(3S)-3-(1H-1,2,4-triazol-5-yl)pyrrolidine-1-carbonyl]-4-piperidyl]oxymethyl]benzamide;
(3S)-1-[3-[3-[2-Methylsulfonyl-4-(trifluoromethyl)phenyl]-1-bicyclo[1.1.1]pentanyl]azetidine-1-carbonyl]pyrrolidine-3-carboxamide;
(3S)-1-[2-[(4-Chloro-2-methylsulfonyl-phenyl)methyl]-6-azaspiro[3.4]octane-6-carbonyl]pyrrolidine-3-carboxamide;
[(3S)-3-(1H-1,2,4-Triazol-5-yl)pyrrolidin-1-yl]-[6-[3-(trifluoromethoxy)phenyl]sulfonyl-2-azaspiro[3.3]heptan-2-yl]methanone;
[(3S)-3-(1H-1,2,4-Triazol-5-yl)pyrrolidin-1-yl]-[7-[3-(trifluoromethyl)phenyl]sulfonyl-2-azaspiro[3.5]nonan-2-yl]methanone;
[(3S)-3-(1H-1,2,4-Triazol-5-yl)pyrrolidin-1-yl]-[7-[4-(trifluoromethyl)phenyl]sulfonyl-2-azaspiro[3.5]nonan-2-yl]methanone;
[3-[3-(4-Fluorophenyl)-1-bicyclo[1.1.1]pentanyl]azetidin-1-yl]-[(3S)-3-(1H-1,2,4-triazol-5-yl)pyrrolidin-1-yl]methanone;
[3-[3-(4-Methylsulfonylphenyl)-1-bicyclo[1.1.1]pentanyl]azetidin-1-yl]-[(3S)-3-(1H-1,2,4-triazol-5-yl)pyrrolidin-1-yl]methanone;
[(3S)-3-(1H-1,2,4-Triazol-5-yl)pyrrolidin-1-yl]-[6-[[4-(trifluoromethylsulfonimidoyl)phenyl]methyl]-2-azaspiro[3.3]heptan-2-yl]methanone;
[(3S)-3-(1H-1,2,4-Triazol-5-yl)pyrrolidin-1-yl]-[6-[[3-(trifluoromethylsulfonimidoyl)phenyl]methyl]-2-azaspiro[3.3]heptan-2-yl]methanone;
[6-[3-(Methylsulfonimidoyl)-5-(trifluoromethyl)phenyl]methyl]-2-azaspiro[3.3]heptan-2-yl]-[rac-(3S)-3-(1H-1,2,4-triazol-5-yl)pyrrolidin-1-yl]methanone;
[3-[[2-Fluoro-4-(trifluoromethylsulfonimidoyl)phenyl]methoxy]azetidin-1-yl]-[rac-(3S)-3-(1H-1,2,4-triazol-5-yl)pyrrolidin-1-yl]methanone;
[(3S)-3-(1H-1,2,4-Triazol-5-yl)pyrrolidin-1-yl]-[4-[5-(trifluoromethyl)pyrazin-2-yl]amino]cuban-1-yl]methanone;
[(3S)-3-(1H-1,2,4-Triazol-5-yl)pyrrolidin-1-yl]-[6-[4-(trifluoromethylsulfonyl)phenyl]methyl]-2-azaspiro[3.3]heptan-2-yl]methanone;
[rac-(3S)-3-(1H-1,2,4-Triazol-5-yl)pyrrolidin-1-yl]-[6-[[3-(trifluoromethoxy)phenyl]sulfonimidoyl]-2-azaspiro[3.3]heptan-2-yl]methanone;
[6-[[3-Fluoro-5-(trifluoromethylsulfonimidoyl)phenyl]methyl]-2-azaspiro[3.3]heptan-2-yl]-[rac-(3S)-3-(1H-1,2,4-triazol-5-yl)pyrrolidin-1-yl]methanone;
[rac-(3S)-3-(1H-1,2,4-Triazol-5-yl)pyrrolidin-1-yl]-[7-[[4-(trifluoromethyl)phenyl]sulfonimidoyl]-2-azaspiro[3.5]nonan-2-yl]methanone;
[rac-(3S)-3-(1H-1,2,4-Triazol-5-yl)pyrrolidin-1-yl]-[7-[3-(trifluoromethyl)phenyl]sulfonimidoyl]-2-azaspiro[3.5]nonan-2-yl]methanone;
[3-[4-[2-(Methylsulfonimidoyl)phenyl]phenyl]azetidin-1-yl]-[rac-(3S)-3-(1H-1,2,4-triazol-5-yl)pyrrolidin-1-yl]methanone;
[3-[(8R)-8-Methyl-8-oxo-8lambda6-thia-9-azatricyclo[8.4.0.02,7]tetradeca-1(10),2 (7),3,5,8,11,13-heptaen-12-yl]azetidin-1-yl]-[(3S)-3-(1H-1,2,4-triazol-5-yl)pyrrolidin-1-yl]methanone;
[3-[(8S)-8-Methyl-8-oxo-8lambda6-thia-9-azatricyclo[8.4.0.02,7]tetradeca-1(10),2(7),3,5,8,11,13-heptaen-12-yl]azetidin-1-yl]-[(3S)-3-(1H-1,2,4-triazol-5-yl)pyrrolidin-1-yl]methanone;
[3-[3-(4-Fluoro-2-methylsulfonyl-phenyl)-1-bicyclo[1.1.1]pentanyl]azetidin-1-yl]-[(3S)-3-(1H-1,2,4-triazol-5-yl)pyrrolidin-1-yl]methanone;
[(3S)-3-(1H-1,2,4-Triazol-5-yl)pyrrolidin-1-yl]-[3-[3-[3-(trifluoromethoxy)phenyl]-1-bicyclo[1.1.1]pentanyl]azetidin-1-yl]methanone;
[(3S)-3-(4H-1,2,4-Triazol-3-yl)pyrrolidin-1-yl]-[4-[5-(trifluoromethyl)-2-pyridyl]methyl]piperazin-1-yl]methanone;
[4-[(2-Methyloxazol-4-yl)methyl]piperazin-1-yl]-[(3S)-3-(1H-1,2,4-triazol-5-yl)pyrrolidin-1-yl]methanone;
[4-[(4-Methylthiazol-5-yl)methyl]piperazin-1-yl]-[(3S)-3-(1H-1,2,4-triazol-5-yl)pyrrolidin-1-yl]methanone;
[4-[(5-Chloro-1,3-dimethyl-pyrazol-4-yl)methyl]piperazin-1-yl]-[(3S)-3-(1H-1,2,4-triazol-5-yl)pyrrolidin-1-yl]methanone;
4-[2-[(3S)-3-(1H-1,2,4-Triazol-5-yl)pyrrolidine-1-carbonyl]-2-azaspiro[3.3]heptan-6-yl]oxy]-2-(trifluoromethyl)benzonitrile;
5-Fluoro-2-[3-[1-[(3S)-3-(1H-1,2,4-triazol-5-yl)pyrrolidine-1-carbonyl]azetidin-3-yl]-1-bicyclo[1.1.1]pentanyl]benzonitrile;
[3-[3-(3-Methyl-1H-pyrazol-5-yl)-1-bicyclo[1.1.1]pentanyl]azetidin-1-yl]-[(3S)-3-(1H-1,2,4-triazol-5-yl)pyrrolidin-1-yl]methanone;
[6-[1-Methyl-4-(trifluoromethyl)pyrazol-3-yl]methyl]-2-azaspiro[3.3]heptan-2-yl]-[(3S)-3-(4H-1,2,4-triazol-3-yl)pyrrolidin-1-yl]methanone;
[(3S)-3-(1H-1,2,4-Triazol-5-yl)pyrrolidin-1-yl]-[3-[3-[4-(trifluoromethylsulfonyl)phenyl]-1-bicyclo[1.1.1]pentanyl]azetidin-1-yl]methanone;
[3-[3-(4-Chloro-2-methylsulfonyl-phenyl)-1-bicyclo[1.1.1]pentanyl]azetidin-1-yl]-[(3S)-3-(1H-1,2,4-triazol-5-yl)pyrrolidin-1-yl]methanone;
[6-[(5-Chloro-1,3-dimethyl-pyrazol-4-yl)methyl]-2,6-diazaspiro[3.3]heptan-2-yl]-[(3S)-3-(1H-1,2,4-triazol-5-yl)pyrrolidin-1-yl]methanone;
5-[2-[(3S)-3-(1H-1,2,4-Triazol-5-yl)pyrrolidine-1-carbonyl]-2-azaspiro[3.3]heptan-6-yl]oxy]-2-(trifluoromethyl)benzonitrile;
[6-[(5-Fluoro-2-pyridyl)oxy]-2-azaspiro[3.3]heptan-2-yl]-[(3S)-3-(1H-1,2,4-triazol-5-yl)pyrrolidin-1-yl]methanone;
[6-[1-Methyl-3-(trifluoromethyl)pyrazol-4-yl]oxy-2-azaspiro[3.3]heptan-2-yl]-[(3S)-3-(1H-1,2,4-triazol-5-yl)pyrrolidin-1-yl]methanone;
[(3S)-3-(1H-1,2,4-Triazol-5-yl)pyrrolidin-1-yl]-[3-[[5-(trifluoromethyl)pyrazin-2-yl]amino]-1-bicyclo[1.1.1]pentanyl]methanone;
(3S)-3-(1H-1,2,4-triazol-5-yl)pyrrolidin-1-yl]-[6-[4-(trifluoromethylsulfonimidoyl) phenoxy]-2-azaspiro[3.3]heptan-2-yl]methanone;
[(3S)-3-(1H-triazol-5-yl)pyrrolidin-1-yl]-[6-[3-(trifluoromethylsulfonimidoyl)phenyl]methyl]-2-azaspiro[3.3]heptan-2-yl]methanone;
[(3S)-3-(1H-triazol-5-yl)pyrrolidin-1-yl]-[6-[3-(trifluoromethylsulfonimidoyl)phenyl]methyl]-2-azaspiro[3.3]heptan-2-yl]methanone;
[(3S)-3-(1H-triazol-5-yl)pyrrolidin-1-yl]-[6-[3-(trifluoromethylsulfonyl)phenyl]methyl]-2-azaspiro[3.3]heptan-2-yl]methanone;
5-[2-[(3S)-3-(1H-triazol-5-yl)pyrrolidine-1-carbonyl]-2-azaspiro[3.3]heptan-6-yl]methyl]-2-(trifluoromethyl)benzonitrile;
(3S)-1-[6-[1-methyl-5-(trifluoromethyl)pyrazol-3-yl]methyl]-2-azaspiro[3.3]heptane-2-carbonyl]pyrrolidine-3-carboxamide;
(3S)-1-[6-[1-methyl-5-(trifluoromethyl)pyrazol-4-yl]methyl]-2-azaspiro[3.3]heptane-2-carbonyl]pyrrolidine-3-carboxamide;
(3S)-1-[6-[[3-(trifluoromethylsulfonyl)phenyl]methyl]-2-azaspiro[3.3]heptane-2-carbonyl]pyrrolidine-3-carboxamide;
(3S)-1-[6-[[4-cyano-3-(trifluoromethyl)phenyl]methyl]-2-azaspiro[3.3]heptane-2-carbonyl]pyrrolidine-3-carboxamide;
(3S)-1-[6-[6-(trifluoromethoxy)-3-pyridyl]methyl]-2-azaspiro[3.3]heptane-2-carbonyl]pyrrolidine-3-carboxamide;
5-[2-[(3S)-3-(1H-1,2,4-triazol-5-yl)pyrrolidine-1-carbonyl]-2-azaspiro[3.3]heptan-6-yl]methyl]-2-(trifluoromethyl)benzonitrile;
[(3S)-3-(1H-triazol-5-yl)pyrrolidin-1-yl]-[6-[4-(trifluoromethylsulfonimidoyl)phenyl]methyl]-2-azaspiro[3.3]heptan-2-yl]methanone;
[(3S)-3-(1H-triazol-5-yl)pyrrolidin-1-yl]-[6-[4-(trifluoromethylsulfonimidoyl)phenyl]methyl]-2-azaspiro[3.3]heptan-2-yl]methanone;
rac-(3S)-1-[6-[[2-fluoro-4-(trifluoromethylsulfonimidoyl)phenyl]methyl]-2-azaspiro[3.3]heptane-2-carbonyl]pyrrolidine-3-carboxamide;
[6-[2-fluoro-4-(trifluoromethylsulfonimidoyl)phenyl]methyl]-2-azaspiro[3.3]heptan-2-yl]-[rac-(3S)-3-(1H-triazol-4-yl)pyrrolidin-1-yl]methanone;
[(3S)-3-(1H-triazol-4-yl)pyrrolidin-1-yl]-[6-[4-(trifluoromethylsulfonyl)phenyl]methyl]-2-azaspiro[3.3]heptan-2-yl]methanone;
[6-[[3-fluoro-5-(trifluoromethylsulfonyl)phenyl]methyl]-2-azaspiro[3.3]heptan-2-yl]-[(3S)-3-(1H-triazol-4-yl)pyrrolidin-1-yl]methanone;
[(3S)-3-(1H-triazol-4-yl)pyrrolidin-1-yl]-[6-[5-(trifluoromethyl)-1H-pyrazol-3-yl]methyl]-2-azaspiro[3.3]heptan-2-yl]methanone; and
[6-[[2-fluoro-4-(trifluoromethylsulfonimidoyl)phenyl]methyl]-2-azaspiro[3.3]heptan-2-yl]-[rac-(3S)-3-(1H-1,2,4-triazol-3-yl)pyrrolidin-1-yl]methanone.
   In a preferred embodiment, the present invention provides a compound of formula (I) as described herein, or a pharmaceutically acceptable salt thereof, selected from:
[3-[[2-Fluoro-4-(trifluoromethyl)phenyl]methoxy]azetidin-1-yl]-[3-(1H-1,2,4-triazol-5-yl)pyrrolidin-1-yl]methanone;
[3-[[2-Fluoro-4-(trifluoromethyl)phenyl]methoxy]azetidin-1-yl]-[(3S)-3-(1H-1,2,4-triazol-5-yl)pyrrolidin-1-yl]methanone;
1-[3-[[2-Fluoro-4-(trifluoromethyl)phenyl]methoxy]azetidine-1-carbonyl]pyrrolidine-3-carboxamide;
[3-[[2-Fluoro-4-(trifluoromethyl)phenyl]methoxy]azetidin-1-yl]-[3-(1H-triazol-5-yl)pyrrolidin-1-yl]methanone;
(+)- or (−)-[2-(2-Chloro-4-fluoro-phenoxy)-7-azaspiro[3.5]nonan-7-yl]-[(3R)-3-(1H-1,2,4-triazol-5-yl)pyrrolidin-1-yl]methanone;
[3-[[2-Fluoro-4-(trifluoromethyl)phenyl]methoxy]azetidin-1-yl]-[3-(1H-imidazol-5-yl)pyrrolidin-1-yl]methanone;
[3-[[2-Fluoro-4-(trifluoromethyl)phenyl]methoxy]azetidin-1-yl]-[(3S)-3-(1H-triazol-5-yl)pyrrolidin-1-yl]methanone;
[3-[[2-Fluoro-4-(trifluoromethyl)phenyl]methoxy]azetidin-1-yl]-[(3R)-3-(1H-triazol-5-yl)pyrrolidin-1-yl]methanone;
[3-[[3-Chloro-4-(trifluoromethyl)phenyl]methoxy]azetidin-1-yl]-[3-(1H-1,2,4-triazol-5-yl)pyrrolidin-1-yl]methanone;
[3-[[2-Chloro-4-(trifluoromethyl)phenyl]methoxy]azetidin-1-yl]-[3-(1H-1,2,4-triazol-5-yl)pyrrolidin-1-yl]methanone;
(−)- or (+)-[3-[2,4-bis(Trifluoromethyl)phenyl]methoxy]azetidin-1-yl]-[3-(1H-1,2,4-triazol-5-yl)pyrrolidin-1-yl]methanone;
(+)- or (−)-1-[3-[4-(2,4-Difluorophenyl)phenyl]azetidine-1-carbonyl]pyrrolidine-3-carboxamide;
(+)- or (−)-[3-(1H-Triazol-5-yl)pyrrolidin-1-yl]-[3-[4-[1-(trifluoromethyl)cyclopropyl]phenyl]azetidin-1-yl]methanone;
(−)- or (+)-[3-(1H-Triazol-5-yl)pyrrolidin-1-yl]-[3-[4-[1-(trifluoromethyl)cyclopropyl]phenyl]azetidin-1-yl]methanone; and
4-[1-[3-[4-[1-(Trifluoromethyl)cyclopropyl]phenyl]azetidine-1-carbonyl]pyrrolidin-3-yl]oxazolidin-2-one.

In a particular embodiment, the present invention provides pharmaceutically acceptable salts of the compounds according to formula (I) as described herein, especially hydrochloride salts. In a further particular embodiment, the present invention provides compounds according to formula (I) as described herein as free bases.

In some embodiments, the compounds of formula (I) are isotopically-labeled by having one or more atoms therein replaced by an atom having a different atomic mass or mass number. Such isotopically-labeled (i.e., radiolabeled) compounds of formula (I) are considered to be within the scope of this disclosure. Examples of isotopes that can be incorporated into the compounds of formula (I) include isotopes of hydrogen, carbon, nitrogen, oxygen, phosphorous, sulfur, fluorine, chlorine, and iodine, such as, but not limited to, ²H, ³H, ¹¹C, ¹³C, ¹⁴C, ¹³N, ¹⁵N, ¹⁵O, ¹⁷O, ¹⁸O, ³¹P, ³²P, ³⁵S, ¹⁸F, ³⁶Cl, ¹²³I, and ¹²⁵I, respectively. Certain isotopically-labeled compounds of formula (I), for example, those incorporating a radioactive isotope, are useful in drug and/or substrate tissue distribution studies. The radioactive isotopes tritium, i.e. ³H, and carbon-14, i.e., ¹⁴C, are particularly useful for this purpose in view of their ease of incorporation and ready means of detection. For example, a compound of formula (I) can be enriched with 1, 2, 5, 10, 25, 50, 75, 90, 95, or 99 percent of a given isotope.

Substitution with heavier isotopes such as deuterium, i.e. ²H, may afford certain therapeutic advantages resulting from greater metabolic stability, for example, increased in vivo half-life or reduced dosage requirements.

Substitution with positron emitting isotopes, such as ¹¹C, ¹⁸F, ¹⁵O and ¹³N, can be useful in Positron Emission Topography (PET) studies for examining substrate receptor occupancy. Isotopically-labeled compounds of formula (I) can generally be prepared by conventional techniques known to those skilled in the art or by processes analogous to those described in the Examples as set out below using an appropriate isotopically-labeled reagent in place of the non-labeled reagent previously employed.

### Processes of Manufacturing

The preparation of compounds of formula (I) of the present invention may be carried out in sequential or convergent synthetic routes. Syntheses of the invention are shown in the following general schemes. The skills required for carrying out the reaction and purification of the resulting products are known to those persons skilled in the art. The substituents and indices used in the following description of the processes have the significance given herein, unless indicated to the contrary.

If one of the starting materials, intermediates or compounds of formula (I) contain one or more functional groups which are not stable or are reactive under the reaction conditions of one or more reaction steps, appropriate protective groups (as described e.g., in “Protective Groups in Organic Chemistry” by T. W. Greene and P. G. M. Wutts, 5th Ed., 2014, John Wiley & Sons, N.Y.) can be introduced before the critical step applying methods well known in the art. Such protective groups can be removed at a later stage of the synthesis using standard methods described in the literature.

If starting materials or intermediates contain stereogenic centers, compounds of formula (I) can be obtained as mixtures of diastereomers or enantiomers, which can be separated by methods well known in the art e.g., chiral HPLC, chiral SFC or chiral crystallization. Racemic compounds can e.g., be separated into their antipodes via diastereomeric salts by crystallization with optically pure acids or by separation of the antipodes by specific chromatographic methods using either a chiral adsorbent or a chiral eluent. It is equally possible to separate starting materials and intermediates containing stereogenic centers to afford diastereomerically/enantiomerically enriched starting materials and intermediates. Using such diastereomerically/enantiomerically enriched starting materials and intermediates in the synthesis of compounds of formula (I) will typically lead to the respective diastereomerically/enantiomerically enriched compounds of formula (I).

A person skilled in the art will acknowledge that in the synthesis of compounds of formula (I)—insofar not desired otherwise—an “orthogonal protection group strategy” will be applied, allowing the cleavage of several protective groups one at a time each without affecting other protective groups in the molecule. The principle of orthogonal protection is well known in the art and has also been described in literature (e.g. Barany and R. B. Merrifield, J. Am. Chem. Soc. 1977, 99, 7363; H. Waldmann et al., Angew. Chem. Int. Ed. Engl. 1996, 35, 2056).

A person skilled in the art will acknowledge that the sequence of reactions may be varied depending on reactivity and nature of the intermediates.

In more detail, the compounds of formula (I) can be manufactured by the methods given below, by the methods given in the examples or by analogous methods. Appropriate reaction conditions for the individual reaction steps are known to a person skilled in the art. Also, for reaction conditions described in literature affecting the described reactions see for example: Comprehensive Organic Transformations: A Guide to Functional Group Preparations, 2nd Edition, Richard C. Larock. John Wiley & Sons, New York, NY. 1999). It was found convenient to carry out the reactions in the presence or absence of a solvent. There is no particular restriction on the nature of the solvent to be employed, provided that it has no adverse effect on the reaction or the reagents involved and that it can dissolve the reagents, at least to some extent. The described reactions can take place over a wide range of temperatures, and the precise reaction temperature is not critical to the invention. It is convenient to carry out the described reactions in a temperature range between −78° C. to reflux. The time required for the reaction may also vary widely, depending on many factors, notably the reaction temperature and the nature of the reagents. However, a period of from 0.5 hours to several days will usually suffice to yield the described intermediates and compounds. The reaction sequence is not limited to the one displayed in the schemes, however, depending on the starting materials and their respective reactivity, the sequence of reaction steps can be freely altered.

If starting materials or intermediates are not commercially available or their synthesis not described in literature, they can be prepared in analogy to existing procedures for close analogues or as outlined in the experimental section.

### The Following Abbreviations are Used in the Present Text:

AcOH=acetic acid, ACN=acetonitrile, Bn=benzyl, Boc=tert-butyloxycarbonyl, CAS RN=chemical abstracts registration number, Cbz=benzyloxycarbonyl, CPME=cyclopentyl methyl ether, Cs₂CO₃=cesium carbonate, CO=carbon monoxide, CuCl=copper (I) chloride, CuCN=copper (I) cyanide, CuI=copper (I) iodide, DAST=(diethylamino) sulfur trifluoride, DCM=DCM, DBU=1,8-diazabicyclo[5,4,0]undec-7-ene, DEA=diethyl amine, DEAD=diethyl azodicarboxylate, DIAD=diisopropyl azodicarboxylate, DMAP=4-dimethylaminopyridine, DME=dimethoxyethane, DMEDA=N,N′-Dimethyl-1,2-ethanediamine, DMF=N,N-Dimethylformamide, DIPEA=N,N-diisopropylethylamine, dppf=1,1 bis(diphenyl phosphino)ferrocene, EDC·HCl=N-(3-dimethylaminopropyl)-N′-ethylcarbodiimide hydrochloride, EI=electron impact, ESI=electrospray ionization, EtOAc=ethyl acetate, EtOH=ethanol, h=hour(s), FA=formic acid, H₂O=water, H₂SO₄=sulfuric acid, HATU=1-[bis(dimethylamino)methylene]-1H-1,2,3-triazolo[4,5-b]pyridinium-3-oxide hexafluorophosphate, HBTU=O-benzotriazole-N,N,N′,N′-tetramethyl-uronium-hexafluorophosphate, HCl=hydrogen chloride, HOBt=1-hydroxy-1H-benzotriazole; HPLC=high performance liquid chromatography, iPrMgCl=isopropylmagnesium chloride, I₂=iodine, IPA=2-propanol, ISP=ion spray positive (mode), ISN=ion spray negative (mode), K₂CO₃=potassium carbonate, KHCO₃=potassium bicarbonate, KI=potassium iodide, KOtBu=potassium tert-butoxide, KOH=potassium hydroxide, K₃PO₄=potassium phosphate tribasic, LiAlH₄ or LAH=lithium aluminium hydride, LiHMDS=lithium bis(trimethylsilyl)amide, LiOH=lithium hydroxide, mCPBA=meta-chloroperoxybenzoic acid, MgSO₄=magnesium sulfate, min=minute(s), mL=milliliter, MPLC=medium pressure liquid chromatography, MS=mass spectrum, MTBE-methyl tert-butyl ether, nBuLi=n-butyllithium, NaBH₃CN=sodium cyanoborohydride, NaH=sodium hydride, NaHCO₃=sodium hydrogen carbonate, NaNO₂=sodium nitrite, NaBH(OAc)₃=sodium triacetoxyborohydride, NaOH=sodium hydroxide, Na₂CO₃=sodium carbonate, Na₂SO₄=sodium sulfate, Na₂S₂O₃=sodium thiosulfate, NBS=N-bromosuccinimide, nBuLi=n-butyllithium, NEt₃=triethylamine (TEA), NH₄Cl=ammonium chloride, NMP=N-methyl-2-pyrrolidone, OAc=Acetoxy, T₃P=propylphosphonic anhydride, PE=petroleum ether, PG=protective group, Pd—C=palladium on activated carbon, PdCl₂(dppf)-DCM=1,1′-bis(diphenylphosphino)ferrocene-palladium(II) dichloride DCM complex, Pd₂(dba)₃=tris(dibenzylideneacetone)dipalladium(0), Pd(OAc)₂=palladium(II) acetate, Pd(OH)₂=palladium hydroxide, Pd(PPh₃)₄=tetrakis(triphenylphosphine)palladium(0), PTSA=p-toluenesulfonic acid, R=any group, RT=room temperature, SFC=Supercritical Fluid Chromatography, S—PHOS=2-dicyclohexylphosphino-2′,6′-dimethoxybiphenyl, TBAI=tetra butyl ammonium iodine, TBME=tert-butyl methyl ether, TEA=triethylamine, TFA=trifluoracetic acid, THF=tetrahydrofuran, TMEDA=N,N,N′,N′-tetramethylethylenediamine, ZnCl₂=zinc chloride, Hal=halogen.

Compounds of formula IA wherein A, B, L, R¹, R², R³, R⁴, p and q are as described herein can be synthesized in analogy to literature procedures and/or as depicted for example in Scheme 1.

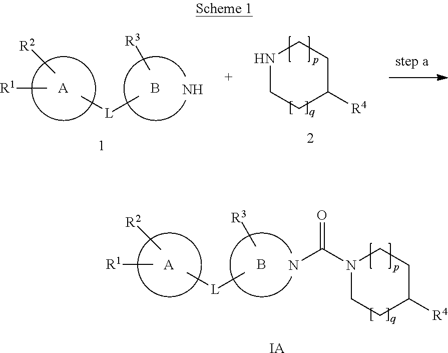

Accordingly, compounds 1 are reacted with intermediates 2 in the presence of an urea forming reagent such as bis(trichloromethyl) carbonate using a suitable base and solvent such as, e.g. sodium bicarbonate in DCM, to give compounds of formula IA (step a). Further urea forming reagents include but are not limited to phosgene, trichloromethyl chloroformate, (4-nitrophenyl) carbonate, 1,1′-carbonyl-di-(1,2,4-triazole) or 1,1′-carbonyldiimidazole. Reactions of this type and the use of these reagents are widely described in literature (e.g. G. Sartori et al., Green Chemistry 2000, 2, 140). A person skilled in the art will acknowledge that the order of the addition of the reagents can be important in this type of reactions due to the reactivity and stability of the intermediary formed carbamoyl chlorides, as well as for avoiding formation of undesired symmetrical urea by-products.

Compounds of formula IA can be obtained as mixtures of diastereomers or enantiomers, which can be separated by methods well known in the art e.g., chiral HPLC, chiral SFC or chiral crystallization. Racemic compounds can e.g., be separated into their antipodes via diastereomeric salts by crystallization with optically pure acids or by separation of the antipodes by specific chromatographic methods using either a chiral adsorbent or a chiral eluent.

In case R⁴ is a 5-membered heteroaromatic system containing a Nitrogen atom that can be alkylated, compound IA can be reacted using a base such as Na₂CO₃, K₂CO₃, NaH or KotBu with a suitable alkylating agent such as iodomethane in a suitable solvent such as THF or DMF to yield compounds IA.

Compounds of formula IB wherein A, B, L, R¹, R², R³, R⁴, p and q are as described herein can be synthesized in analogy to literature procedures and/or as depicted for example in Scheme 2.

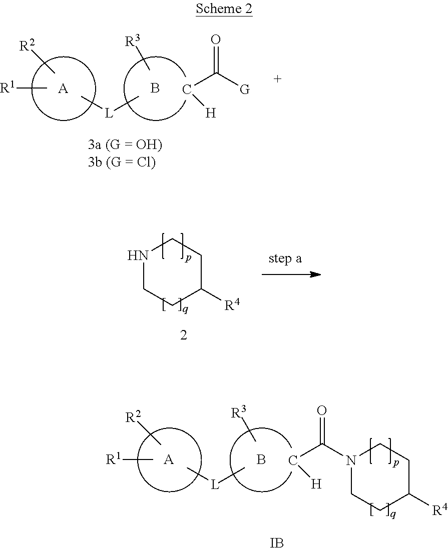

Accordingly, intermediates 2 can be coupled with an activated form of a carboxylic acid 3a (G═OH) or alternatively with carboxylic acid chlorides 3b (G═Cl) to provide compounds IB(step a). Amide couplings of this type with carboxylic acids are widely described in the literature and can be accomplished by the usage of coupling reagents such as CDI, DCC, HATU, HBTU, HOBT, TBTU, T3P or Mukaiyama reagent (Mukaiyama T. Angew. Chem., Int. Ed. Engl. 1979, 18, 707) in a suitable solvent e.g., DMF, DMA, DCM or dioxane, optionally in the presence of a base (e.g., TEA, DIPEA (Huenig's base) or DMAP).

Alternatively, the carboxylic acids 3a can be converted into their acid chlorides 3b by treatment with, e.g. thionyl chloride or oxalyl chloride, neat or optionally in a solvent such as DCM. Subsequent reaction of the acid chloride with intermediates 1 in an appropriate solvent such as DCM or DMF and a base, e.g. TEA, Huenig's base, pyridine, DMAP or lithium bis(trimethylsilyl)amide at temperatures ranging from 0° C. to the reflux temperature of the solvent or solvent mixture yields compounds IB(step a).

Compounds of formula IB can be obtained as mixtures of diastereomers or enantiomers, which can be separated by methods well known in the art e.g., chiral HPLC, chiral SFC or chiral crystallization. Racemic compounds can e.g., be separated into their antipodes via diastereomeric salts by crystallization with optically pure acids or by separation of the antipodes by specific chromatographic methods using either a chiral adsorbent or a chiral eluent.

In case R⁴ is a 5-membered heteroaromatic system containing a Nitrogen atom that can be alkylated compound IB using a base such as Na₂CO₃, K₂CO₃, NaH or KotBu was reacted with a suitable alkylating agent such as iodomethane to yield compounds IB.

Compounds of formula IC wherein A, B, R¹, R², R³, R⁴, p and q are as described herein can be synthesized in analogy to literature procedures and/or as depicted for example in Scheme 3.

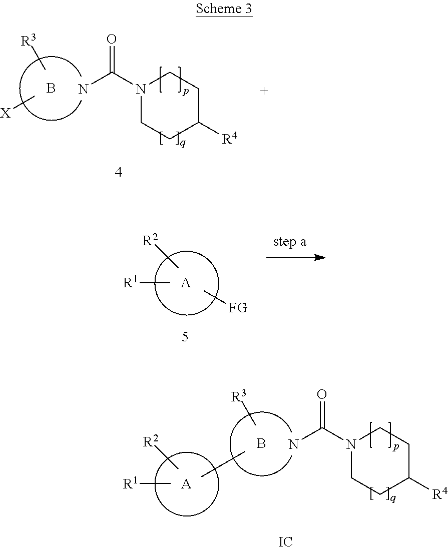

Accordingly, intermediates 4, either commercially available or prepared by methods known in the art, in which X is bromide or iodide can be subjected to cross-coupling reactions such as Suzuki, Buchwald-Hartwig, Negishi, Heck, Stille, or Sonogashira coupling reactions with intermediates 5, either commercially available or prepared by methods known in the art.

For example, intermediates 4 can be reacted with intermediates 5 in which FG signifies with aryl or heteroaryl boronic acids (FG═B(OH)₂) or boronic esters (FG=e.g. 4,4,5,5-tetramethyl-2-phenyl-1,3,2-dioxaborolane (pinacol) ester) either commercially available or prepared using literature procedures as described for example in “Boronic Acids-Preparation and Applications in Organic Synthesis and Medicine” by Dennis G. Hall (ed.) 1st Ed., 2005, John Wiley & Sons, New York) using a suitable catalyst (e.g. dichloro[1,1′-bis(diphenylphosphino)-ferrocene]palladium(II) DCM adduct, tetrakis(triphenylphosphine)palladium(0) or palladium(II) acetate with triphenylphosphine) in an appropriate solvent (e.g. dioxane, dimethoxyethane, water, toluene, DMF or mixtures thereof) and a suitable base (e.g. Na2CO3, NaHCO3, KF, K2CO3 or TEA) at temperatures between room temperature and the boiling point of the solvent or solvent mixture, to yield compounds IC (step a). Suzuki reactions of this type are broadly described in literature (e.g. A. Suzuki, Pure Appl. Chem. 1991, 63, 419-422; A. Suzuki, N. Miyaura, Chem. Rev. 1995, 95, 2457-2483; A. Suzuki, J. Organomet. Chem. 1999, 576, 147-168; V. Polshettiwar et al., Chem. Sus. Chem. 2010, 3, 502-522) and are well known to those skilled in the art. Alternatively, aryl- or heteroaryl-trifluoroborates 19c (FG=BF3) can be used in the cross-coupling reaction applying a palladium catalyst such as, e.g. tetrakis(triphenylphosphine)-palladium(0), palladium(II) acetate or dichloro[1,1′-bis(diphenylphosphino)ferrocene]-palladium(II) DCM adduct in the presence of a suitable base such as cesium carbonate or potassium phosphate in solvents such as toluene, THF, dioxane, water or mixtures thereof, at temperatures between room temperature and the boiling point of the solvent or solvent mixture (step a).

For example, intermediates 4 can be reacted with intermediates 5 in which FG is Sn(alkyl) 3 and alkyl is preferable n-butyl or methyl, using a suitable catalyst and solvent such as, e.g. tetrakis(triphenylphosphine)-palladium(0) in DMF at temperatures between room temperature and the boiling point of the solvent or solvent mixture to provide intermediates 10 (step a). Stille reactions of that type are well known in the art and described in literature, e.g. Org. React. 1997, 50, 1-652, ACS Catal. 2015, 5, 3040-3053.

For example, intermediates 4 can be reacted with intermediates 5 in which FG is ZnHal and Hal preferably bromide or iodide, either commercially available or prepared by literature methods, using an appropriate catalyst and solvent system such as, e.g. [1,1′-bis(diphenylphosphino)ferrocene]dichloropalladium(II) and copper (I) iodide in DMA, or tetrakis(triphenylphosphine)palladium(0) in THF or DMF at temperatures between room temperature and the boiling point of the solvent to provide intermediates 10 (step a). Negishi reactions of that type are well known in the art and also described in literature, e.g. Org. Lett., 2005, 7, 4871, ACS Catal. 2016, 6, 1540-1552. Acc. Chem. Res. 1982, 15, pp 340-348. Alternatively, intermediates 10 may be prepared by first converting intermediates 8 in which X is for example iodide into the corresponding zinc species by applying literature methods (e.g. reaction of 8 with Zn powder in the presence of chlorotrimethylsilane and 1,2-dibromoethane in a suitable solvent such as DMA) and subsequent coupling of the zinc species with aryl- or heteroarylbromides- or iodides under the conditions mentioned before.

Alternatively, intermediates 4 in which X is preferably bromide can be subjected to a cross-electrophile coupling with aryl- or heteroarylbromides 5 in which FG signifies bromide under irradiation with a 420 nm blue light lamp using an appropriate photo catalyst such as [Ir{dF(CF₃)ppy}₂(dtbpy)]PF₆ ([4,4′-bis(1,1-dimethylethyl)-2,2′-bipyridine-N1,N1′]bis[3,5-difluoro-2-[5-(trifluoromethyl)-2-pyridinyl-N]phenyl-C]Iridium (III) hexafluorophosphate), a Nickel catalyst like NiCl₂ glyme (dichloro(dimethoxyethane) nickel), 4,4′-di-tert-butyl-2,2′-dipyridyl and tris(trimethylsilyl) silane, in the presence of a suitable base such as anhydrous sodium carbonate in a solvent like DME. Reactions of this type are described in literature, e.g. J. Am. Chem. Soc. 2016, 138, 8084 (step a).

Alternatively, intermediates 4 in which X is iodide, bromide, chloride or OTf can be reacted with intermediates 5, in which FG signifies a functional group, such as R—C≡C—H or R—C≡C—H applying one of the cross-coupling methods described before to provide compounds IC (step a).

Compounds of formula IC can be obtained as mixtures of diastereomers or enantiomers, which can be separated by methods well known in the art e.g., chiral HPLC, chiral SFC or chiral crystallization. Racemic compounds can e.g., be separated into their antipodes via diastereomeric salts by crystallization with optically pure acids or by separation of the antipodes by specific chromatographic methods using either a chiral adsorbent or a chiral eluent.

In case R⁴ is a 5-membered heteroaromatic system containing a Nitrogen atom that can be alkylated compound IC using a base such as Na₂CO₃, K₂CO₃, NaH or KotBu was reacted with a suitable alkylating agent such as iodomethane to yield compounds IC.

In some embodiments, intermediates 1 are intermediates of type 1a. Intermediates 1a in which R¹, R², R³, A and B are as described herein can be prepared by a methods known in the art and as exemplified by the general synthetic procedure outlined in Scheme 4.

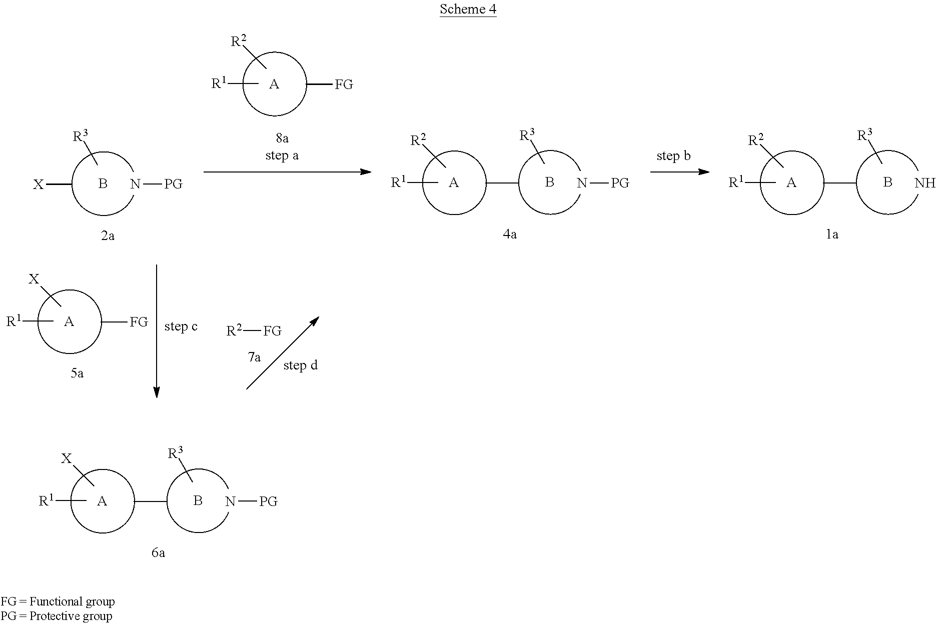

Intermediates 2a, either commercially available or prepared by methods known in the art, in which PG signifies a suitable protecting group and X is bromide or iodide can be subjected to cross-coupling reactions such as Negishi, Heck, Stille, Suzuki, Sonogashira or Buchwald-Hartwig coupling reactions with compounds 8a, either commercially available or prepared by methods known in the art, in which FG signifies a suitable functional group such as, e.g. chloro, bromo, iodo, —OSO₂ alkyl (e.g. mesylate (methanesulfonic acid)), —OSO₂ fluoroalkyl (e.g. triflate (trifluoromethanesulfonic acid)) or —OSO₂ aryl (e.g. tosylate (p-toluenesulfonic acid)). Reactions of this type are broadly described in literature and well known to persons skilled in the art (step a).

For example, intermediates 2a in which X is preferably bromide or iodide can be reacted with intermediates 8a, in which FG signifies a functional group, such as boronic acids (FG=B(OH)₂) or boronic esters (FG=e.g. 4,4,5,5-tetramethyl-2-phenyl-1,3,2-dioxaborolane (pinacol) ester) either commercially available or prepared using literature procedures as described for example in “Boronic Acids-Preparation and Applications in Organic Synthesis and Medicine” by Dennis G. Hall (ed.) 1st Ed., 2005, John Wiley & Sons, New York) using a suitable catalyst (e.g. dichloro[1,1′-bis(diphenylphosphino)-ferrocene]palladium(II) DCM adduct, tetrakis(triphenylphosphine)palladium(0) or palladium(II) acetate with triphenylphosphine) in an appropriate solvent (e.g. dioxane, dimethoxyethane, water, toluene, DMF or mixtures thereof) and a suitable base (e.g. Na₂CO₃, NaHCO₃, KF, K₂CO₃ or TEA) at temperatures between room temperature and the boiling point of the solvent or solvent mixture, to yield intermediates 4a (step a). Suzuki reactions of this type are broadly described in literature (e.g. A. Suzuki, Pure Appl. Chem. 1991, 63, 419-422; A. Suzuki, N. Miyaura, Chem. Rev. 1995, 95, 2457-2483; A. Suzuki, J. Organomet. Chem. 1999, 576, 147-168; V. Polshettiwar et al., Chem. Sus. Chem. 2010, 3, 502-522) and are well known to those skilled in the art. Alternatively, intermediates 8a, in which FG signifies a functional group, such as trifluoroborate (FG=BF3) can be used in the cross-coupling reaction applying a palladium catalyst such as, e.g. tetrakis(triphenylphosphine)-palladium(0), palladium(II) acetate or dichloro[1,1′-bis(diphenylphosphino)ferrocene]-palladium(II) DCM adduct in the presence of a suitable base such as cesium carbonate or potassium phosphate in solvents such as toluene, THF, dioxane, water or mixtures thereof, at temperatures between room temperature and the boiling point of the solvent or solvent mixture

### (Step a)

Alternatively, intermediates 2a can be reacted with intermediates 8a, in which FG signifies a functional group, such as stannane in which FG is Sn(alkyl)₃ and alkyl is preferable n-butyl or methyl, using a suitable catalyst and solvent such as, e.g. tetrakis(triphenylphosphine)-palladium(0) in DMF at temperatures between room temperature and the boiling point of the solvent or solvent mixture to provide intermediates 4a (step a). Stille reactions of that type are well known in the art and described in literature, e.g. Org. React. 1997, 50, 1-652, ACS Catal. 2015, 5, 3040-3053.

Furthermore, intermediates 2a can be reacted with intermediates 8a, in which FG signifies a functional group, such as zinc halides (FG=ZnHal and Hal preferably Br or I), either commercially available or prepared by literature methods, using an appropriate catalyst and solvent system such as, e.g. [1,1′-bis(diphenylphosphino)ferrocene]dichloropalladium(II) and copper (I) iodide in DMA, or tetrakis(triphenylphosphine)palladium(0) in THF or DMF at temperatures between room temperature and the boiling point of the solvent to provide intermediates 4a (step a). Negishi reactions of that type are well known in the art and also described in literature, e.g. Org. Lett., 2005, 7, 4871, ACS Catal. 2016, 6, 1540-1552. Acc. Chem. Res. 1982, 15, pp 340-348. Alternatively, intermediates 10 may be prepared by first converting intermediates 2a in which X is for example iodide into the corresponding zinc species by applying literature methods (e.g. reaction of 2a with Zn powder in the presence of chlorotrimethylsilane and 1,2-dibromoethane in a suitable solvent such as DMA) and subsequent coupling of the zinc species with aryl- or heteroarylbromides- or iodides under the conditions mentioned before.

Alternatively, intermediates 2a in which X is preferably bromide can be subjected to a cross-electrophile coupling with intermediates 8a, in which FG signifies a functional group, such as bromide (FG=Br) under irradiation with a 420 nm blue light lamp using an appropriate photo catalyst such as [Ir{dF(CF₃)ppy}₂(dtbpy)]PF₆ ([4,4′-bis(1,1-dimethylethyl)-2,2′-bipyridine-N1,N1′]bis[3,5-difluoro-2-[5-(trifluoromethyl)-2-pyridinyl-N]phenyl-C]Iridium (III) hexafluorophosphate), a Nickel catalyst like NiCl₂ glyme (dichloro(dimethoxyethane) nickel), 4,4′-di-tert-butyl-2,2′-dipyridyl and tris(trimethylsilyl) silane, in the presence of a suitable base such as anhydrous sodium carbonate in a solvent like DME. Reactions of this type are described in literature, e.g. J. Am. Chem. Soc. 2016, 138, 8084 (step a).

Furthermore, intermediates 2a in which X is preferably iodide can be subjected to Suzuki-Miyaura cross coupling reactions with arylboronic acids 8a (FG=B(OH)₂) using a suitable Nickel catalyst such as nickel (II) iodide in the presence of rac-(1R,2R)-2-aminocyclohexan-1-ol and a suitable base such as sodium bis(trimethylsilyl)amide in an appropriate solvent like iPrOH, dioxane, THF or DME, preferably iPrOH, at temperatures between room temperature and the boiling point of the solvent, optionally applying microwave heating, to yield intermediates 4a. Reactions of this type are described in literature, e.g. ChemistrySelect. 2017, 2, 8841 (step a).

Alternatively, intermediates 2a can be reacted with intermediates 8a, in which FG signifies a functional group, such as an amine, using a suitable catalyst and solvent such as, e.g. tris(dibenzylideneacetone) dipalladium(0) chloroform adduct and X-Phos or bis(diphenylphosphino)-1,1′-binaphthalene and palladium(II) acetate in tert-butanol or 1,4-dioxane and a suitable base such as Cs2CO3 or KOtBu at temperatures between room temperature and the boiling point of the solvent or solvent mixture to provide intermediates 4a (step a). Buchwald Hartwig reactions of that type are well known in the art and described in literature, e.g. Angewandte Chemie. 2019, 58, 17118-17129.

For example, intermediates 2a in which X is preferably bromide or iodide can be reacted with intermediates 5a, in which FG signifies a functional group, such as boronic acids (FG=B(OH)₂) or boronic esters (FG=e.g. 4,4,5,5-tetramethyl-2-phenyl-1,3,2-dioxaborolane (pinacol) ester) and X═F either commercially available or prepared using literature procedures as described for example in “Boronic Acids-Preparation and Applications in Organic Synthesis and Medicine” by Dennis G. Hall (ed.) 1st Ed., 2005, John Wiley & Sons, New York) using a suitable catalyst (e.g. dichloro[1,1′-bis(diphenylphosphino)-ferrocene]palladium(II) DCM adduct, tetrakis(triphenylphosphine)palladium(0) or palladium(II) acetate with triphenylphosphine) in an appropriate solvent (e.g. dioxane, dimethoxyethane, water, toluene, DMF or mixtures thereof) and a suitable base (e.g. Na₂CO₃, NaHCO₃, KF, K₂CO₃ or TEA) at temperatures between room temperature and the boiling point of the solvent or solvent mixture, to yield intermediates 6a (step c). Suzuki reactions of this type are broadly described in literature (e.g. A. Suzuki, Pure Appl. Chem. 1991, 63, 419-422; A. Suzuki, N. Miyaura, Chem. Rev. 1995, 95, 2457-2483; A. Suzuki, J. Organomet. Chem. 1999, 576, 147-168; V. Polshettiwar et al., Chem. Sus. Chem. 2010, 3, 502-522) and are well known to those skilled in the art.

Intermediates 6a can be reacted under nucleophilic substitution conditions with intermediates 7a in which FG=S⁻Na⁺ and R2=Me in an appropriate solvent like DMSO at a temperature between 0° C. and 100° C. to get compounds 4a (step d).

Alternatively, intermediates of 4 a obtained in such a manner can be oxidized using reactants such as m-Chloroperbenzoic acid in appropriate solvents such as DCM at a temperature between 0° C. and 25° C. to get compounds 4a.

Removal of the protective group from intermediates 4a in which PG signifies a protective groups such as tert-butyloxycarbonyl (BOC) can be performed using methods well known in the art and as described (see “Protective Groups in Organic Chemistry” by T. W. Greene and P. G. M. Wutts, 5th Ed., 2014, John Wiley & Sons, New York), e.g., using TFA or hydrochloric acid in a solvent like DCM, 1,4-dioxane or THF preferable at room temperature or 4-methylbenzenesulfonic acid hydrate in a solvent like EtOAc preferably at elevated temperatures, furnishes intermediates 1a (step b).

In some embodiments, intermediates 1 are intermediates of type 1b. Intermediates 1b in which R¹, R², R³, R⁴, A and B are as described herein can be prepared by a variety of conditions, which may be exemplified by the general synthetic procedure outlined in Scheme 5.

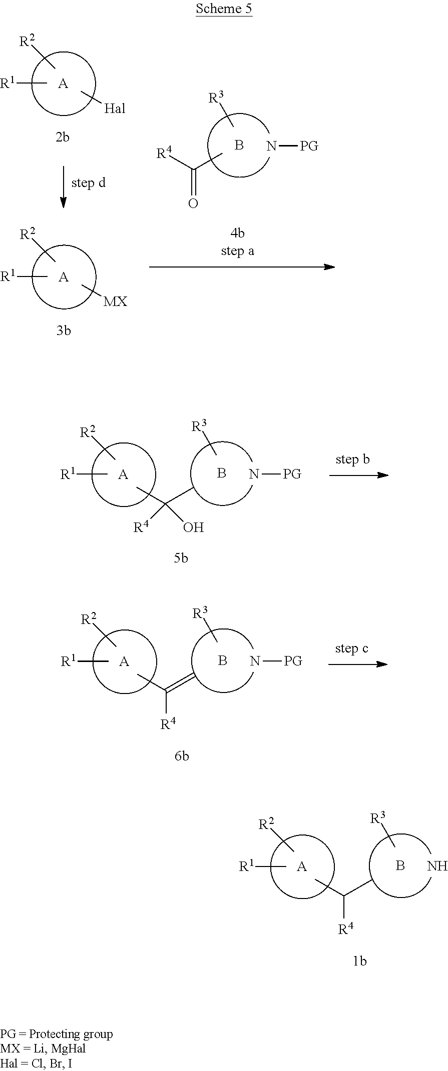

Addition of an organometallic compound of type 3b in which MX is for example Li or MgCl, MgBr or MgI to intermediates 4b in which PG is a suitable protective group, e.g. a Boc group, provides intermediates 5b (step a). Reactions of this type are well known in the art and described in literature (J. Med. Chem. 1989, 32(1), 105, J. Med. Chem. 2014, 57 (4), 1543, Bioorg. Med. Chem Lett. 2015, 25 (13), 2720). In case compounds 3b in which MX=MgHal with Hal being Cl, Br or I (Grignard reagents) are commercially not available they may be prepared for example by reaction of the corresponding aryl or heteroaryl halides 2b with magnesium in a suitable solvent such as THF, optionally in the presence of catalytic amounts of iodine at temperatures ranging from 0° C. to the boiling point of the solvent (step d). Alternatively, a lithium halogen exchange reaction can be performed with aryl or heteroaryl halides 2b using a solution of LiHMDS or n-BuLi, preferably n-BuLi in a solvent like THF, diethyl ether, n-pentane, n-hexane or mixtures thereof, preferably THF and in a temperature range between −20° C. and −78° C., preferably at −78° C., to generate the corresponding lithiated aryl or heteroaryl intermediate (M=Li). Nucleophilic addition of the in situ prepared lithiated aryl or heteroaryl intermediate to ketones of type 4b in which PG is a suitable protecting group such as a Boc group in a solvent such as THF and preferably at a temperature of −78° C. gives the corresponding tertiary alcohols 5b (step a).

Subsequent elimination of the tertiary hydroxy group in intermediates 5b, optionally with concomitant removal of an acid labile protective group (e.g. a Boc protective group) using acidic conditions such as 4 M HCl in dioxane in a solvent like MeOH, or, preferably, TFA in DCM, yields the corresponding olefinic intermediates 6b (step b).

Heterogeneous catalytic hydrogenation of olefins 6b using a catalyst such as Pd(OH)₂ or Pd/C in a solvent like THF, MeOH, EtOH, EtOAc or a mixture thereof, preferably Pd/C in THF under e.g., atmospheric pressure of hydrogen, affords intermediates of type 1b (step c).

Intermediates 4b are commercially available and/or can be prepared in analogy to methods described in literature, e.g. Bioorg. Med. Chem. Lett. 2011, 21 (18), 5191, WO2012/155199, WO2016/180536, Bioorg. Med. Chem. Lett. 2008, 18 (18), 5087, WO2007/117557, J. Am. Chem. Soc. 2017, 139 (33), 11353, J. Med. Chem. 2017, 60 (13), 5507.

In some embodiments, intermediates 1 are intermediates of type 1c. Intermediates 1c in which R¹, R², R³, A and B are as described herein can be prepared by a methods well known in the art and as exemplified by the general synthetic procedure outlined in Scheme 6.

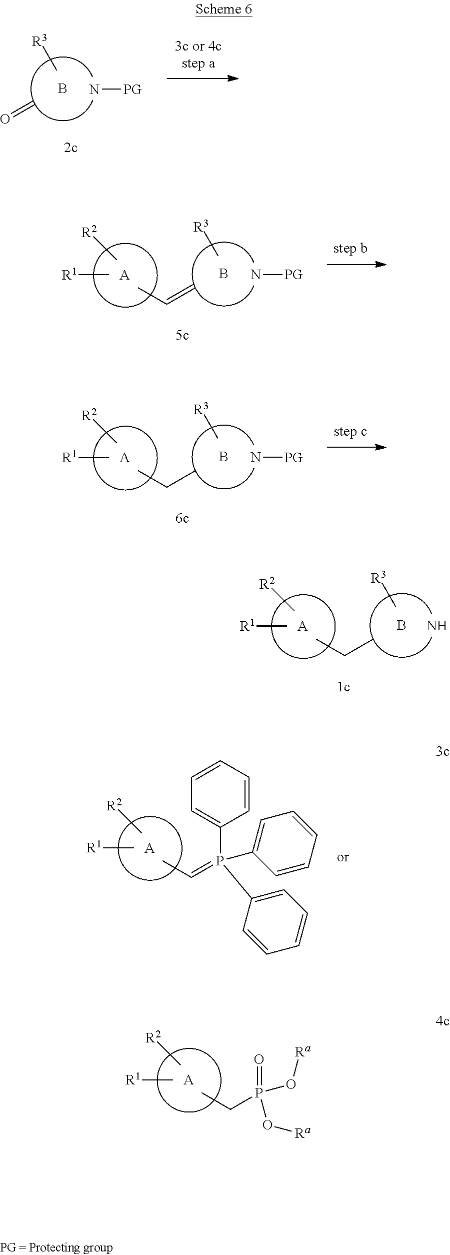

Ketones 2c in which PG is a suitable protective group, either commercially available or prepared by methods known in the art, can be subjected for example to a Wittig reaction with alkylidene triphenylphosphoranes of type 3c in a suitable solvent such as, e.g. THF, Methyl-THF or DMSO to give intermediates 5c (step a). Phosphoranes 3c can be formed by treating the corresponding phosphonium salts with a suitable base such as BuLi, NaH, or KOtBu in a suitable solvent such as THF, dioxane or Methyl-THF and may be isolated or used in situ. Phosphonium salts in turn are readily available from an aryl/heteroaryl/heterocyclic-substituted alkylhalide (with halide being Cl, Br and iodo) and triphenylphosphine in a suitable solvent such as toluene. Heating may be applied to accelerate the reaction or drive the reaction to completion (e.g. H. J. Cristau, F. Plénat in PATAI'S Chemistry of Functional Groups, Editor(s): Frank R. Hartley, August 2006, Series Editor(s): Prof. Saul Patai).

Alternatively, intermediates 5c can be obtained using a Horner-Wadsworth-Emmons (HWE) reaction using ketones 2c and phosphonates 4c, wherein R^{a} is alkyl, for example methyl or ethyl. Phosphonates 4c are in situ α-metalated using a suitable base and solvent such as NaH, nBuLi or KOtBu in THF (step a). Phosphonates 4c are readily prepared using for example the Arbuzov reaction by alkylation of an aryl/heteroaryl/heterocyclic halide (with halide being Cl, Br and iodo) with commercially available trialkyl phosphite (e.g. Chem. Rev. 1984, 84, 577).

Olefination reactions of both types are broadly described in literature (e.g. Current Org. Chem. 2015, 19 (9), page 744; Chem. Rev. 1989, 89 (4), 863; Org. React. 1977, 25, 73; Liebigs Ann./Recueil 1997, 1283; Acc. Chem. Res. 1983, 16, 411).

Reduction of the double bond in intermediates 5c using, e.g. hydrogen in the presence of a suitable catalyst such as palladium on charcoal in an appropriate solvent or solvent mixture such as EtOAc, MeOH or AcOH yields compounds 6c (step b).

Removal of the protective group from intermediates 6c applying methods known in the art (e.g., a Boc group using TFA in DCM or 4 M HCl in dioxane at temperatures between 0° C. and room temperature, a Cbz group using hydrogen in the presence of a suitable catalyst such as Pd or Pd(OH)₂ on charcoal in a suitable solvent such as MeOH, EtOH, EtOAc or mixtures thereof and as described for example in “Protective Groups in Organic Chemistry” by T. W. Greene and P. G. M. Wuts, 4th Ed., 2006, Wiley N.Y.), furnishes intermediates 1c (step c).

In some embodiments, intermediates 1 are intermediates of type 1d. Intermediates 1d in which R¹, R², R³, A and B are as described herein can be prepared by a methods well known in the art and as exemplified by the general synthetic procedure outlined in Scheme 7.

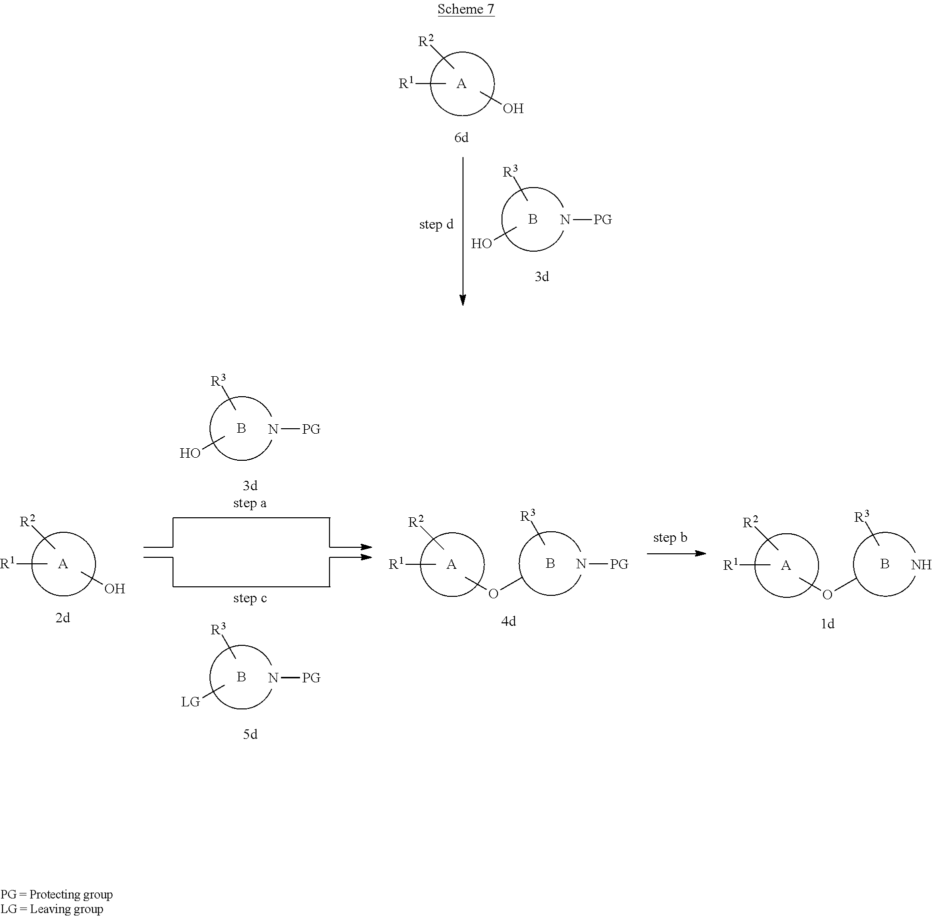

Alcohols of type 2d can be subjected to a Mitsunobu reaction with intermediates 3d in which PG is a suitable protective group such as a Cbz, Boc or Bn, using an appropriate phosphine such as triphenylphosphine and a dialkyl azodicarboxylate such as DEAD or DIAD in a suitable solvent such as THF to give intermediates 4d (step a). Mitsunobu reactions of that type are broadly described in literature (e.g. Org. Chem. Front. 2015, 2, 739; Chem. Rev. 2009, 109 (6), 2551).

Alternatively, intermediates 4d may be prepared from alcohols 2d that can be alkylated with compounds 5d in which LG is a suitable leaving group such as chlorine, bromine, iodine, OSO₂ alkyl (e.g. methanesulfonic acid), OSO₂ fluoroalkyl (e.g. trifluoromethanesulfonic acid) or OSO₂ aryl (e.g. p-toluenesulfonic acid using a suitable base such as NaH or Cs₂CO₃ in an appropriate solvent such as DMF or ACN at temperatures between 0° C. and the boiling temperature of the solvent (step c).

Furthermore, intermediates 4d may be synthesized via alkylation of alcohols of type 3d with compounds 6d under the conditions described under step c (step d).

Removal of the protective group from intermediates 4d applying literature methods and as described for example under Scheme 4, step b, furnishes intermediates 1d (step b).

In some embodiments, intermediates 1 are intermediates of type 1e. Intermediates 1e in which R¹, R², R³, A and B are as described herein can be prepared by a variety of conditions, which may be exemplified by the general synthetic procedure outlined in Scheme 8.

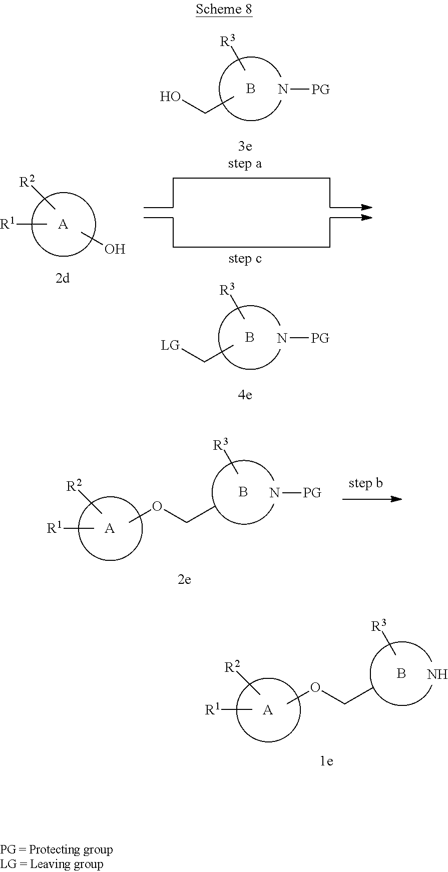

Alcohols of type 2d can be subjected to a Mitsunobu reaction with intermediates 3e in which PG is a suitable protective group such as a Cbz, Boc or Bn, using an appropriate phosphine such as triphenylphosphine and a dialkyl azodicarboxylate such as DEAD or DIAD in a suitable solvent such as THF, DCM or ACN to give intermediates 2e (step a). Mitsunobu reactions of that type are broadly described in literature (e.g. Org. Chem. Front. 2015, 2, 739; Chem. Rev. 2009, 109 (6), 2551).

Removal of the protective group from intermediates 2e applying literature methods and as described for example under Scheme 4, step b, furnishes intermediates 1e (step b).

Alternatively, intermediates 2e may be prepared from alcohols 2d that can be alkylated with compounds 4e in which LG is a suitable leaving group such as chlorine, bromine, iodine, OSO₂ alkyl (e.g. methanesulfonic acid), OSO₂ fluoroalkyl (e.g. trifluoromethanesulfonic acid) or OSO₂ aryl (e.g. p-toluenesulfonic acid) using a suitable base such as Cs₂CO₃, Huenig's base or NaH, in an appropriate solvent, such as DMF at temperatures between 0° C. and the boiling temperature of the solvent (step c).

In some embodiments, intermediates 1 are intermediates of type 1f. Intermediates 1f in which R¹, R², R³, A and B are as described herein can be prepared by a methods known in the art and as exemplified by the general synthetic procedure outlined in Scheme 9.

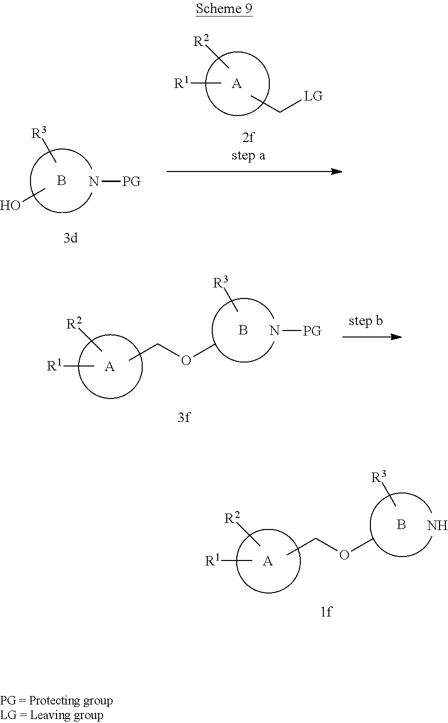

Intermediates 3f may be prepared from alcohols 3d, either commercially available or prepared by methods known by a person skilled in the art and in which PG is a suitable protective group such as a Cbz, Boc or Bn, by alkylation with compounds 2f in which LG is a suitable leaving group such as chlorine, bromine, iodine, OSO₂ alkyl (e.g. methanesulfonic acid), OSO₂ fluoroalkyl (e.g. trifluoromethanesulfonic acid) or OSO₂ aryl (e.g. p-toluenesulfonic acid) using a suitable base, such as sodium hydride, Huenig's base or potassium tert-butoxide, in an appropriate solvent (e.g. in DMF or THF) at temperatures between 0° C. and the boiling temperature of the solvent (step a).

Removal of the protective group from intermediates 3f applying methods known in the art (e.g., a Boc group using TFA in DCM at temperatures between 0° C. and room temperature, a Cbz group using hydrogen in the presence of a suitable catalyst such as Pd or Pd(OH)₂ on charcoal in a suitable solvent such as MeOH, EtOH, EtOAc or mixtures therefore and as described for example in “Protective Groups in Organic Chemistry” by T. W. Greene and P. G. M. Wuts, 4th Ed., 2006, Wiley N.Y.), furnishes intermediates 1f (step b).

In some embodiments, intermediates 1 are intermediates of type 1g. Intermediates of type 1g in which R¹, R², R³, A and B are as described herein can be prepared by methods well known by a person skilled in the art and as exemplified by the general synthetic procedures outlined in

### Scheme 9.

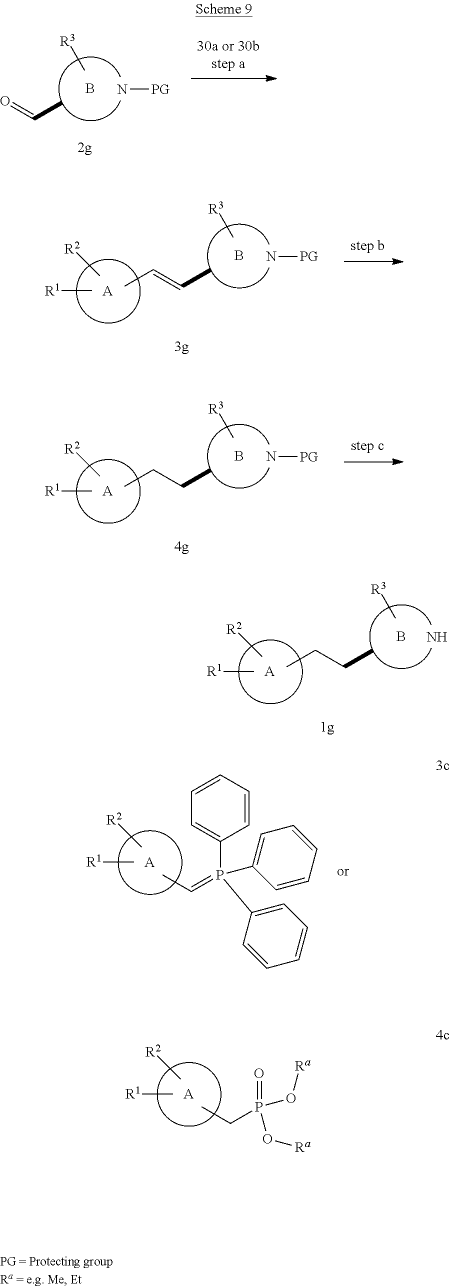

Aldehydes 2g, either commercially available or prepared by methods known in the art, can be subjected to a Wittig reaction or Horner-Wadsworth-Emmons (HWE) reaction using alkylidene triphenylphosphoranes of type 3c and phosphonates 4c, respectively, using for example the conditions described under Scheme 6, step a, to give intermediates 3g (step a).

Reduction of the double bond in intermediates 3g applying literature conditions or the conditions described under Scheme 5, step c or Scheme 6, step b, yields compounds 4g (step b).

Removal of the protective group from intermediates 4g applying methods known in the art and as outlined under Scheme 4, step b, furnishes intermediates 1g (step c).

In another embodiment, intermediates 1 are intermediates of type 1h. Intermediates of type 1h in which R¹, R², R³, A and B are as described herein, can be prepared by methods well known in the art and as exemplified by the general synthetic procedures outlined in Scheme 10

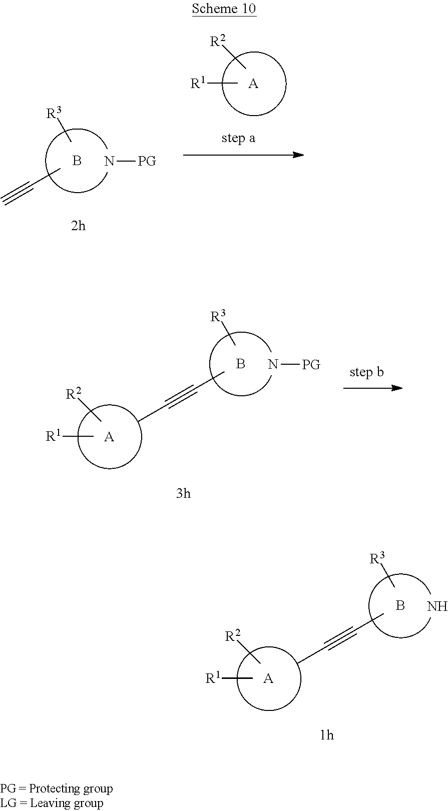

Alkynes 2h, either commercially available or prepared by methods known in the art and in which PG signifies a suitable protecting group such as, e.g. a Boc, Cbz or Bn protecting group, can be subjected to a Sonogashira reaction (e.g. Chem. Soc. Rev. 2011, 40, 5084) with aryl or heteroaryl halides 3h, wherein LG is preferably Br, I or OTf and using a suitable catalyst, such as, Pd(OAc)₂/PPh₃, Pd(PPh₃)₄, preferably PdCh(PPh₃)₄ in presence of Cui and an appropriate base such as, e.g. K₂CO₃, CS₂CO₃, DIPEA or preferably TEA and suitable solvent such as, e.g. THE, DMSO, DMF, NMP, CH₃CN or dioxane, preferably THF and in a temperature range between room temperature and 100° C., preferably around 65° C. to give intermediates 9 (step a).

Removal of the protective group from intermediates 3h, applying methods known in the art, e.g., a Boc group using TFA in DCM or 4 M HCl in dioxane at temperatures between 0° C. and room temperature, a Cbz group using hydrogen in the presence of a suitable catalyst such as Pd or Pd(OH)₂ on charcoal in a suitable solvent such as MeOH, EtOH, EtOAc or mixtures thereof and as described for example in “Protective Groups in Organic Chemistry” by T. W. Greene and P. G. M. Wuts, 4th Ed., 2006, Wiley N.Y.), furnishes intermediates 1h (step b).

In some embodiments, intermediates 3 are intermediates of type 3i. Intermediates 3i in which R¹, R², R³, A and B are as described herein can be prepared by a methods known in the art and as exemplified by the general synthetic procedure outlined in Scheme 11.

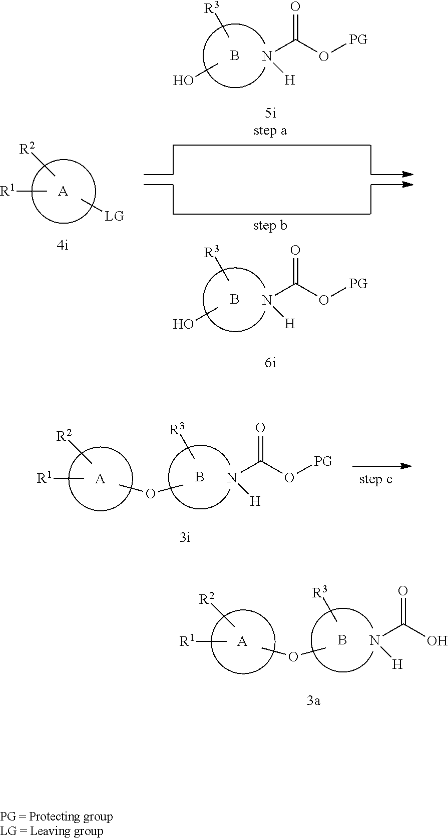

Intermediates 3i may be prepared from alcohols 5i, either commercially available or prepared by methods known by a person skilled in the art and in which PG is a suitable protective group such as a Methyl, Ethyl or tert-Butyl, by alkylation with compounds 4i in which FG is a suitable leaving group such as chlorine, bromine, iodine, OSO₂ alkyl (e.g. methanesulfonic acid), OSO₂ fluoroalkyl (e.g. trifluoromethanesulfonic acid) or OSO₂ aryl (e.g. p-toluenesulfonic acid) using a suitable base, such as sodium hydride, Huenig's base or potassium tert-butoxide, in an appropriate solvent (e.g. in DMF or THF) at temperatures between 0° C. and the boiling temperature of the solvent (step a).

Intermediates 3i may be prepared from aldehydes or ketones 6i, either commercially available or prepared by methods known by a person skilled in the art and in which PG is a suitable protective group such as a Methyl, Ethyl or tert-Butyl, by reductive etherification with compounds 4i in which FG is a Hydroxy group using suitable reagents, such as e.g. Et3 SiH and Bismuth (III) chloride, in an appropriate solvent (e.g. in DCM) at temperatures between 0° C. and the boiling temperature of the solvent or using conditions as described in literature (e.g. by S. Hatakeyama in Tetrahedron Letters, Vol. 35, No. 25, pp. 4367-4370, 1994)(step b).

Removal of the protective group from intermediates 3i applying methods known in the art (e.g., a Methyl group using LiOH or NaOH in mixtures of water and e.g. THF or 1,4-Dioxane at temperatures between 0° C. and room temperature, a tert-Butyl group using suitable acids such as HCl or TFA in a suitable solvent such as AcOH, DCM or mixtures therefore and as described for example in “Protective Groups in Organic Chemistry” by T. W. Greene and P. G. M. Wuts, 4th Ed., 2006, Wiley N.Y.), furnishes intermediates 3a (step c).

In one aspect, the present invention provides a process of manufacturing a compound of formula (IA) or (IB) described herein, comprising:
(a) reacting an amine of formula 2, wherein p, q, and R⁴ are as described herein,

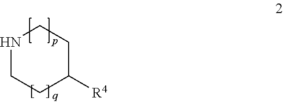

with a carboxylic acid 3a, wherein L, A, B, and R¹ to R³ are as described herein

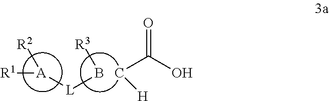

in the presence of a coupling reagent, such as CDI, DCC, HATU, HBTU, HOBT, TBTU, T₃P or Mukaiyama reagent, and optionally in the presence of a base, such as TEA, DIPEA (Huenig's base) or DMAP, to form said compound of formula (IB), wherein A, B, L, p, q, and R¹ to R⁴ are as defined herein

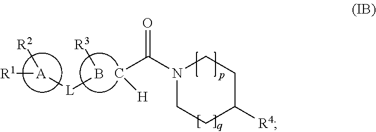

or
(b) reacting an amine of formula 2, wherein p, q, and R⁴ are as described herein,

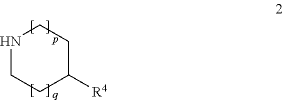

with a carboxylic acid chloride 3b, wherein L, A, B, and R¹ to R³ are as described herein

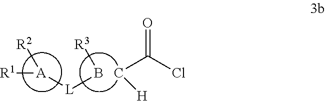

in the presence of a base, such as TEA, Huenig's base, pyridine, DMAP or lithium bis(trimethylsilyl)amide, to form said compound of formula (IB), wherein A, B, L, p, q, and R¹ to R⁴ are as defined herein; or
(c) reacting a first amine of formula 1, wherein A, B, L, and R¹ to R³ are as described herein,

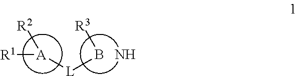

with a second amine 2, wherein X, Y, and Z are as described herein

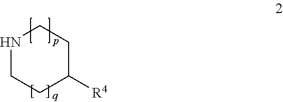

in the presence of a base, such as sodium bicarbonate, and a urea forming reagent, such as bis(trichloromethyl) carbonate, phosgene, trichloromethyl chloroformate, (4-nitrophenyl) carbonate or 1,1′-carbonyldiimidazole, to form said compound of formula (IA), wherein A, B, L, p, q, and R¹ to R⁴ are as defined herein

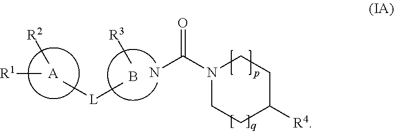

In one aspect, the present invention provides a compound of formula (I) as described herein, when manufactured according to any one of the processes described herein.

### MAGL Inhibitory Activity

Compounds of the present invention are MAGL inhibitors. Thus, in one aspect, the present invention provides the use of compounds of formula (I) as described herein for inhibiting MAGL in a mammal.

In a further aspect, the present invention provides compounds of formula (I) as described herein for use in a method of inhibiting MAGL in a mammal.

In a further aspect, the present invention provides the use of compounds of formula (I) as described herein for the preparation of a medicament for inhibiting MAGL in a mammal.

In a further aspect, the present invention provides a method for inhibiting MAGL in a mammal, which method comprises administering an effective amount of a compound of formula (I) as described herein to the mammal.

Compounds were profiled for MAGL inhibitory activity by determining the enzymatic activity by following the hydrolysis of the natural substrate 2-arachidonoylglycerol resulting in arachidonic acid, which can be followed by mass spectrometry. This assay is hereinafter abbreviated “2-AG assay”.

The 2-AG assay was carried out in 384 well assay plates (PP, Greiner Cat #784201) in a total volume of 20 μL. Compound dilutions were made in 100% DMSO (VWR Chemicals 23500.297) in a polypropylene plate in 3-fold dilution steps to give a final concentration range in the assay from 12.5 μM to 0.8 μM. 0.25 μL compound dilutions (100% DMSO) were added to 9 μL MAGL in assay buffer (50 mM TRIS (GIBCO, 15567-027), 1 mM EDTA (Fluka, 03690-100 ml), 0.01% (v/v) Tween. After shaking, the plate was incubated for 15 min at RT. To start the reaction, 10 μL 2-arachidonoylglycerol in assay buffer was added. The final concentrations in the assay was 50 μM MAGL and 8 μM 2-arachidonoylglycerol. After shaking and 30 min incubation at RT, the reaction was quenched by the addition of 40 μL of acetonitrile containing 4 μM of d8-arachidonic acid. The amount of arachidonic acid was traced by an online SPE system (Agilent Rapidfire) coupled to a triple quadrupole mass spectrometer (Agilent 6460). A C18 SPE cartridge (G9205A) was used in an acetonitrile/water liquid setup. The mass spectrometer was operated in negative electrospray mode following the mass transitions 303.1→259.1 for arachidonic acid and 311.1→267.0 for d8-arachidonic acid. The activity of the compounds was calculated based on the ratio of intensities [arachidonic acid/d8-arachidonic acid].

TABLE 1

| | IC₅₀ MAGL |
|---|---|
| Example | [nM] |
| 1 | 4 |
| 2 | 4 |
| 3 | 146 |
| 4 | 278 |
| 5 | 3 |
| 6 | 1922 |
| 7 | 2966 |
| 8 | 1199 |
| 9 | 37 |
| 10 | 98 |
| 11 | 28 |
| 12 | 22 |
| 13 | 2959 |
| 14 | 1383 |
| 15 | 3 |
| 16 | 28 |
| 17 | 1 |
| 18 | 19 |
| 19 | 506 |
| 20 | 334 |
| 21 | 1204 |
| 22 | 524 |
| 23 | 909 |
| 24 | 208 |
| 25 | 62 |
| 26 | 596 |
| 27 | 3353 |
| 28 | 89 |
| 29 | 212 |
| 30 | 9 |
| 31 | 1220 |
| 32 | 1282 |
| 33 | 939 |
| 34 | 66 |
| 35 | 104 |
| 36 | 1 |
| 37 | 8 |
| 38 | 9 |
| 39 | 984 |
| 40 | 58 |
| 41 | 7 |
| 42 | 1 |
| 43 | 14 |
| 44 | 2010 |
| 45 | 21 |
| 46 | 1885 |
| 47 | 22 |
| 48 | 24 |
| 49 | 5 |
| 50 | 7 |
| 51 | 7 |
| 52 | 31 |
| 53 | 76 |
| 54 | 1 |
| 55 | 5 |
| 56 | 32 |
| 57 | 326 |
| 58 | 6 |
| 59 | 5 |
| 60 | 101 |
| 61 | 2 |
| 62 | 20 |
| 63 | 1126 |
| 64 | 114 |
| 65 | 4 |
| 66 | 203 |
| 67 | 986 |
| 68 | 430 |
| 69 | 1532 |
| 70 | 852 |
| 71 | 656 |
| 72 | 3319 |
| 73 | 4 |
| 74 | 75 |
| 75 | 3786 |
| 76 | 63 |
| 77 | 176 |
| 78 | 1 |
| 79 | 4 |
| 80 | 74 |
| 81 | 32 |
| 82 | 2556 |
| 83 | 1453 |
| 84 | 683 |
| 85 | 18 |
| 86 | 18 |
| 87 | 176 |
| 88 | 2 |
| 89 | 5 |
| 90 | 1247 |
| 91 | 701 |
| 92 | 17 |
| 93 | 494 |
| 94 | 750 |
| 95 | 98 |
| 96 | 4 |
| 97 | 31 |
| 98 | 5 |
| 99 | 36 |
| 100 | 189 |
| 101 | 609 |
| 102 | 215 |
| 103 | 4902 |
| 104 | 238 |
| 105 | 35 |
| 106 | 64 |
| 107 | <1 |
| 108 | 2 |
| 109 | 126 |
| 110 | 15 |
| 111 | 102 |
| 112 | <1 |
| 113 | 1 |
| 114 | 571 |
| 115 | 9 |
| 116 | 5 |
| 117 | 1 |
| 118 | 40 |
| 119 | 1 |
| 120 | <1 |
| 121 | <1 |
| 122 | <1 |
| 123 | 5 |
| 124 | 16 |
| 125 | 118 |
| 126 | 8 |
| 127 | 3 |
| 128 | 2 |
| 129 | 22 |
| 130 | 41 |
| 131 | 21 |
| 132 | 3 |
| 133 | 2 |
| 134 | 274 |
| 135 | 1825 |
| 136 | 4 |
| 137 | 11 |
| 138 | 28 |
| 139 | 1 |
| 140 | 30 |
| 141 | 3 |
| 142 | 85 |
| 143 | 139 |
| 144 | 538 |
| 145 | 79 |
| 146 | 22 |
| 147 | 40 |
| 148 | 2381 |
| 149 | 240 |
| 150 | 49 |
| 151 | 1270 |
| 152 | 222 |
| 153 | 5 |
| 154 | 522 |
| 155 | 83 |
| 156 | 506 |
| 157 | 50 |
| 158 | 231 |
| 159 | 13 |
| 160 | 559 |
| 161 | 24 |
| 162 | 6 |
| 163 | 4 |
| 164 | 924 |
| 165 | 525 |
| 166 | 84 |
| 167 | 85 |
| 168 | 109 |
| 169 | 1272 |
| 170 | 11 |
| 171 | 158 |
| 172 | 56 |
| 173 | 18 |
| 174 | 10 |
| 175 | 36 |
| 176 | 20 |
| 177 | 36 |
| 178 | 98 |
| 179 | 79 |
| 180 | 130 |
| 181 | 4351 |
| 182 | 1 |
| 183 | 2 |
| 184 | 154 |
| 185 | 1659 |
| 186 | 86 |
| 187 | 42 |
| 188 | 3175 |
| 189 | 77 |
| 190 | 117 |
| 191 | 165 |
| 192 | 9 |
| 193 | 4 |
| 194 | 2 |
| 195 | 18 |
| 196 | 0 |
| 197 | 78 |
| 198 | 0 |
| 199 | 0 |
| 200 | 7 |
| 201 | 1 |
| 202 | 35 |
| 203 | 0 |
| 204 | 0 |
| 205 | 4 |
| 206 | 1 |
| 207 | 4964 |
| 208 | 3813 |
| 209 | 38 |
| 210 | 0 |
| 211 | 15 |
| 212 | 5 |
| 213 | 107 |
| 214 | 71 |
| 215 | 19 |
| 216 | 0 |
| 217 | 3 |
| 218 | 0 |
| 219 | 7 |
| 220 | 41 |
| 221 | 14 |
| 222 | 10 |
| 223 | 2 |
| 224 | 1 |
| 225 | 2 |
| 226 | 1 |
| 227 | 0 |
| 228 | 1 |
| 229 | 10 |
| 230 | 18 |
| 231 | 63 |
| 232 | 853 |
| 233 | 53 |
| 234 | 1126 |
| 235 | 17 |
| 236 | 32 |
| 237 | 56 |
| 238 | 55 |
| 239 | 19 |
| 240 | 19 |
| 241 | 2 |
| 242 | 30 |
| 243 | 9 |
| 244 | 268 |
| 245 | 6 |
| 246 | 3410 |
| 247 | 3118 |
| 248 | 1 |
| 249 | 24 |
| 250 | 160 |
| 251 | 32 |
| 252 | 98 |
| 253 | 42 |
| 254 | 45 |
| 255 | 100 |
| 256 | 2222 |
| 257 | 15 |
| 258 | 89 |
| 259 | 6 |
| 260 | 4 |
| 261 | 1 |
| 262 | 243 |
| 263 | 586 |
| 264 | 358 |
| 265 | 1224 |
| 266 | 2352 |
| 267 | 3675 |
| 268 | 33 |
| 269 | 554 |
| 270 | 72 |
| 271 | 2858 |
| 272 | 90 |
| 273 | 1806 |
| 274 | 50 |
| 275 | 923 |
| 276 | 1791 |
| 277 | 1030 |
| 278 | 787 |
| 279 | 234 |
| 280 | 569 |
| 281 | 1399 |
| 282 | 699 |
| 283 | 192 |
| 284 | 810 |
| 285 | 245 |
| 286 | 23 |
| 287 | 2724 |
| 288 | 564 |
| 289 | 1437 |
| 290 | 1268 |
| 291 | 3536 |
| 292 | 44 |
| 293 | 809 |
| 294 | 303 |
| 295 | 1943 |
| 296 | 33 |
| 297 | 382 |
| 298 | 13 |
| 299 | 9 |
| 300 | 2303 |
| 301 | 2812 |
| 302 | 2986 |
| 303 | 2044 |
| 304 | 344 |
| 305 | 13 |
| 306 | 87 |
| 307 | 351 |
| 308 | 769 |
| 309 | 112 |
| 310 | 3 |
| 311 | 209 |
| 312 | 1563 |
| 313 | 1329 |
| 314 | 186 |
| 315 | 1778 |
| 316 | 19 |
| 317 | 3 |
| 318 | 49 |
| 319 | 1141 |
| 320 | 511 |
| 321 | 68 |
| 322 | 93 |
| 323 | 70 |
| 324 | 632 |
| 325 | 125 |
| 326 | 759 |
| 327 | 329 |
| 328 | 16 |
| 329 | 0 |
| 330 | 2 |
| 331 | 1 |
| 332 | 209 |
| 333 | 183 |
| 334 | 0 |
| 335 | 65 |
| 336 | 451 |
| 337 | 1248 |
| 338 | 375 |
| 339 | 211 |
| 340 | 3505 |
| 341 | 2759 |
| 342 | 201 |
| 343 | 1463 |
| 344 | 7 |
| 345 | 278 |
| 346 | 96 |
| 347 | 251 |
| 348 | 713 |
| 349 | 519 |
| 350 | 803 |
| 351 | 1429 |
| 352 | 2200 |
| 353 | 2573 |
| 354 | 4408 |
| 355 | 4757 |
| 356 | 701 |
| 357 | 2482 |
| 358 | 889 |
| 359 | 18 |
| 360 | 407 |
| 361 | 132 |
| 362 | 173 |
| 363 | 441 |
| 364 | 2544 |
| 365 | 3460 |
| 366 | 11 |
| 367 | 414 |
| 368 | 505 |
| 369 | 4415 |
| 370 | 509 |
| 371 | 151 |
| 372 | 4129 |
| 373 | 4430 |
| 374 | 1239 |
| 375 | 1952 |
| 376 | 100 |
| 377 | 100 |
| 378 | 362 |
| 379 | 1260 |
| 380 | 560 |
| 381 | 2375 |
| 382 | 1088 |
| 383 | 122 |
| 384 | 3101 |
| 385 | 4830 |
| 386 | 1780 |
| 387 | 0 |
| 388 | 1010 |
| 389 | 1 |
| 390 | 12 |
| 391 | 424 |
| 392 | 36 |
| 393 | 16 |
| 394 | 0 |
| 395 | 701 |
| 396 | 387 |
| 397 | 2473 |
| 398 | 189 |
| 399 | 369 |
| 400 | 96 |
| 401 | 458 |
| 402 | 0 |
| 403 | 2 |
| 404 | 216 |
| 405 | 16 |
| 406 | 5 |
| 407 | 18 |
| 408 | 170 |
| 409 | 26 |
| 410 | 1334 |
| 411 | 113 |
| 412 | 238 |
| 413 | 22 |
| 414 | 532 |
| 415 | 45 |
| 416 | 962 |
| 417 | 0 |
| 418 | 3 |
| 419 | 135 |
| 420 | 1084 |
| 421 | 571 |
| 422 | 336 |
| 423 | 69 |
| 424 | 37 |
| 425 | 891 |
| 426 | 34 |
| 427 | 29 |
| 428 | 518 |
| 429 | 641 |
| 430 | 123 |
| 431 | 120 |
| 432 | 79 |
| 433 | 258 |
| 434 | 66 |
| 435 | 32 |
| 436 | 49 |
| 437 | 270 |
| 438 | 79 |
| 439 | 205 |
| 440 | 116 |
| 441 | 391 |
| 442 | 2728 |
| 443 | 3169 |
| 444 | 571 |
| 445 | 51 |
| 446 | 119 |
| 447 | 814 |
| 448 | 543 |
| 449 | 310 |
| 450 | 274 |
| 451 | 4906 |
| 452 | 108 |
| 453 | 1030 |
| 454 | 99 |
| 455 | 156 |
| 456 | 227 |
| 457 | 497 |
| 458 | 14 |
| 459 | 19 |
| 460 | 338 |
| 461 | 1940 |
| 462 | 1348 |
| 463 | 355 |
| 464 | 278 |
| 465 | 156 |
| 466 | 105 |
| 467 | 20 |
| 468 | 13 |
| 469 | 578 |
| 470 | 150 |
| 471 | 1785 |
| 472 | 847 |
| 473 | 1919 |
| 474 | 64 |
| 475 | 424 |
| 476 | 47 |
| 477 | 94 |
| 478 | 215 |
| 479 | 1515 |
| 480 | 817 |
| 481 | 892 |
| 482 | 612 |
| 483 | 1661 |
| 484 | 242 |
| 485 | 999 |
| 486 | 41 |
| 487 | 12 |
| 488 | 8 |
| 489 | 2640 |
| 490 | 232 |
| 491 | 2351 |
| 492 | 783 |
| 493 | 520 |
| 494 | 841 |
| 495 | 10 |
| 496 | 1064 |
| 497 | 102 |
| 498 | 677 |
| 499 | 435 |
| 500 | 14 |
| 501 | 1465 |
| 502 | 650 |
| 503 | 486 |
| 504 | 394 |
| 505 | 379 |
| 506 | 2335 |
| 507 | 712 |
| 508 | 540 |
| 509 | 1601 |
| 510 | 281 |
| 511 | 1869 |
| 512 | 86 |
| 513 | 45 |
| 514 | 1 |
| 515 | 169 |
| 516 | 494 |
| 517 | 283 |
| 518 | 4913 |
| 519 | 190 |
| 520 | 4414 |
| 521 | 133 |
| 522 | 1176 |
| 523 | 314 |
| 524 | 113 |
| 525 | 27 |
| 526 | 27 |
| 527 | 79 |
| 528 | 129 |
| 529 | 178 |
| 530 | 216 |
| 531 | 195 |
| 532 | 127 |
| 533 | 180 |
| 534 | 27 |
| 535 | 156 |
| 536 | 158 |
| 537 | 349 |
| 538 | 358 |
| 539 | 503 |
| 540 | 1507 |
| 541 | 399 |
| 542 | 935 |
| 543 | 190 |
| 544 | 70 |
| 545 | 1260 |
| 546 | 255 |
| 547 | 156 |
| 548 | 143 |
| 549 | 26 |
| 550 | 188 |
| 551 | 114 |
| 552 | 36 |
| 553 | 3540 |
| 554 | 2908 |
| 555 | 40 |
| 556 | 28 |
| 557 | 1569 |
| 558 | 13 |
| 559 | 1 |
| 560 | 815 |
| 561 | 2490 |
| 562 | 368 |
| 563 | 42 |
| 564 | 2 |
| 565 | 32 |
| 566 | 3833 |
| 567 | 1696 |
| 568 | 979 |
| 569 | 1500 |
| 570 | 2037 |
| 571 | 4073 |
| 572 | 135 |
| 573 | 140 |
| 574 | 9 |
| 575 | 114 |
| 576 | 630 |
| 577 | 2134 |
| 578 | 3513 |
| 579 | 4981 |
| 580 | 2633 |
| 581 | 102 |
| 582 | 8 |
| 583 | 1 |
| 584 | 4 |
| 585 | 350 |
| 586 | 3927 |
| 587 | 2343 |
| 588 | 3953 |
| 589 | 47 |
| 590 | 102 |
| 591 | 41 |
| 592 | 51 |
| 593 | 44 |
| 594 | 273 |
| 595 | 1026 |
| 596 | 274 |
| 597 | 327 |
| 598 | 869 |
| 599 | 120 |
| 600 | 108 |
| 601 | 1297 |
| 602 | 1165 |
| 603 | 1726 |
| 604 | 374 |
| 605 | 1546 |
| 606 | 376 |
| 607 | 981 |
| 608 | 898 |
| 609 | 1706 |
| 610 | 49 |
| 611 | 107 |
| 612 | 488 |
| 613 | 3980 |
| 614 | 1657 |
| 615 | 281 |
| 616 | 3802 |
| 617 | 1164 |
| 618 | 454 |
| 619 | 330 |
| 620 | 48 |
| 621 | 0 |
| 622 | 681 |
| 623 | 10 |
| 624 | 937 |
| 625 | 555 |
| 626 | 181 |
| 627 | 465 |
| 628 | 9 |
| 629 | 27 |
| 630 | 170 |
| 631 | 312 |
| 632 | 303 |
| 633 | 149 |
| 634 | 257 |
| 635 | 55 |
| 636 | 64 |
| 637 | 79 |
| 638 | 184 |
| 639 | 725 |
| 640 | 11 |
| 641 | 42 |
| 642 | 443 |
| 643 | 37 |
| 644 | 150 |
| 645 | 489 |
| 646 | 122 |
| 647 | 44 |
| 648 | 406 |
| 649 | 88 |
| 650 | 17 |
| 651 | 1847 |
| 652 | 1728 |
| 653 | 2 |
| 654 | 1 |
| 655 | 10 |
| 656 | 941 |
| 657 | 843 |
| 658 | 774 |
| 659 | 2150 |
| 660 | 2998 |
| 661 | 95 |
| 662 | 120 |
| 663 | 647 |
| 664 | 19 |
| 665 | 9 |
| 666 | 761 |
| 667 | 810 |
| 668 | 375 |
| 669 | 346 |
| 670 | 42 |
| 671 | 50 |
| 672 | 29 |
| 673 | 6 |
| 674 | 4634 |
| 675 | 1756 |
| 676 | 280 |
| 677 | 86 |
| 678 | 107 |
| 679 | 26 |
| 680 | 144 |
| 681 | 191 |
| 682 | 406 |
| 683 | 34 |
| 684 | 14 |
| 685 | 4436 |
| 686 | 1776 |
| 687 | 375 |
| 688 | 4126 |
| 689 | 15 |
| 690 | 1801 |
| 691 | 407 |
| 692 | 385 |
| 693 | 97 |
| 694 | 777 |
| 695 | 1 |
| 696 | 2613 |
| 697 | 123 |
| 698 | 349 |
| 699 | 572 |
| 700 | 29 |
| 701 | 199 |
| 702 | 81 |
| 703 | 60 |
| 704 | 428 |
| 705 | 610 |
| 706 | 59 |
| 707 | 2841 |
| 708 | 138 |
| 709 | 203 |
| 710 | 356 |
| 711 | 1081 |
| 712 | 130 |
| 713 | 44 |
| 714 | 59 |
| 715 | 7 |
| 716 | 168 |
| 717 | 263 |
| 718 | 1 |
| 719 | 660 |
| 720 | 332 |
| 721 | 162 |
| 722 | 3047 |
| 723 | 1136 |
| 724 | 445 |
| 725 | 448 |
| 726 | 168 |
| 727 | 681 |
| 728 | 287 |
| 729 | 3852 |
| 730 | 1626 |
| 731 | 1224 |
| 732 | 311 |
| 733 | 85 |
| 734 | 43 |
| 735 | 40 |
| 736 | 13 |
| 737 | 170 |
| 738 | 109 |
| 739 | 48 |
| 740 | 441 |
| 741 | 282 |
| 742 | 320 |
| 743 | 944 |
| 744 | 1148 |
| 745 | 962 |
| 746 | 832 |
| 747 | 4263 |
| 748 | 4256 |
| 749 | 1729 |
| 750 | 1746 |
| 751 | 838 |
| 752 | 492 |
| 753 | 1845 |
| 754 | 47 |
| 755 | 949 |
| 756 | 4001 |
| 757 | 774 |
| 758 | 2956 |
| 759 | 135 |
| 760 | 175 |
| 761 | 74 |
| 762 | 158 |
| 763 | 230 |
| 764 | 310 |
| 765 | 3 |
| 766 | 956 |
| 767 | 266 |
| 768 | 155 |
| 769 | 233 |
| 770 | 1949 |
| 771 | 175 |
| 772 | 118 |
| 773 | 106 |
| 774 | 96 |
| 775 | 48 |
| 776 | 20 |
| 777 | 40 |
| 778 | 139 |
| 779 | 417 |
| 780 | 43 |
| 781 | 61 |
| 782 | 1027 |
| 783 | 489 |
| 784 | 37 |
| 785 | 3186 |
| 786 | 95 |
| 787 | 471 |
| 788 | 59 |
| 789 | 106 |
| 790 | 11 |
| 791 | 20 |
| 792 | 471 |
| 793 | 2199 |
| 794 | 444 |
| 795 | 4230 |
| 796 | 39 |
| 797 | 22 |
| 798 | 10 |
| 799 | 205 |
| 800 | 40 |
| 801 | 693 |
| 802 | 638 |
| 803 | 12 |
| 804 | 13 |
| 805 | 1632 |
| 806 | 4211 |
| 807 | 295 |
| 808 | 4 |
| 809 | 2656 |
| 810 | 2988 |
| 811 | 895 |
| 812 | 413 |
| 813 | 9 |
| 814 | 99 |
| 815 | 44 |
| 816 | 107 |
| 817 | 16 |
| 818 | 39 |
| 819 | 251 |
| 820 | 185 |
| 821 | 10 |
| 822 | 9 |
| 823 | 2207 |
| 824 | 42 |
| 825 | 1116 |
| 826 | 801 |
| 827 | 46 |
| 828 | 80 |
| 829 | 150 |
| 830 | 15 |
| 831 | 1 |
| 832 | 4630 |
| 833 | 211 |
| 834 | 2780 |
| 835 | 437 |
| 836 | 277 |
| 837 | 11 |
| 838 | 220 |
| 839 | 72 |
| 840 | 2 |
| 841 | 4 |
| 842 | 1349 |
| 843 | 137 |
| 844 | 671 |
| 845 | 659 |
| 846 | 194 |
| 847 | 3663 |
| 848 | 1 |
| 849 | 1 |
| 850 | 0 |
| 851 | 0 |
| 852 | 28 |
| 853 | 11 |
| 854 | 6 |
| 855 | 11 |
| 856 | 19 |
| 857 | 20 |
| 858 | 4 |
| 859 | 8 |
| 860 | 4 |
| 861 | 1 |
| 862 | 0 |
| 863 | 0 |
| 864 | 5 |
| 865 | 34 |

In one aspect, the present invention provides compounds of formula (I) and their pharmaceutically acceptable salts or esters as described herein, wherein said compounds of formula (I) and their pharmaceutically acceptable salts or esters have IC₅₀'s for MAGL inhibition below 25 μM, preferably below 10 μM, more preferably below 5 μM as measured in the MAGL assay described herein.

In one embodiment, compounds of formula (I) and their pharmaceutically acceptable salts or esters as described herein have IC₅₀ (MAGL inhibition) values between 0.000001 μM and 25 μM, particular compounds have IC₅₀ values between 0.000005 μM and 10 μM, further particular compounds have IC₅₀ values between 0.00005 μM and 5 μM, as measured in the MAGL assay described herein.

### MAGL NanoBRET™ Target Engagement Assay

Compounds were profiled by a novel cellular MAGL NanoBRET™ Target Engagement assay, which measures the apparent affinity of test compounds by competitive displacement of a Roche-developed Tracer, reversibly bound to a NanoLuc® luciferase MAGL fusion protein in cells.

The Roche-developed Tracer is described in WO2021058443 (Example 21) and has the following structure:

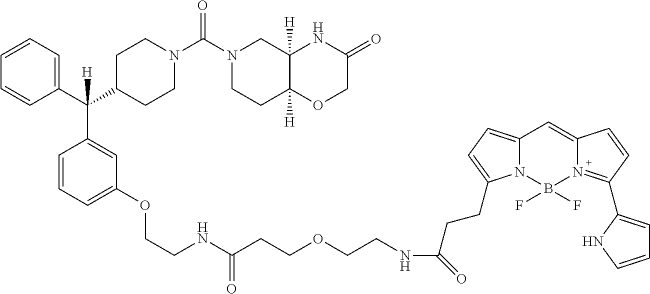

A HEK293A stable cell line expressing NanoLuc_MAGL was established at Roche and assigned as HEK293A_NanoLuc_MAGL clone #43. A fixed concentration of tracer is added to cells expressing the NanoLuc®_MAGL fusion protein to generate a BRET reporter complex. Introduction of competing compounds results in a dose-dependent decrease in NanoBRET™ energy transfer, which allows quantification of the intracellular affinity of the target protein for the test compound.

HEK293A NanoLuc_MAGL clone #43 cells were cultured in DMEM (Gibco 31966) with 10% FBS and 800 μg/ml G418. The NanoBRET assay were performed by seeding 5000 cells/well in Costar low-volume white 384-well plate in assay buffer (1% FBS in HBSS), followed by adding 50 nM final concentration of tracer and serial dilutions of test compounds. After 2 hours incubation, Nanoluciferase substrates and extracellular inhibitors were added following the manufacturer's protocol (Promega, #N2520) and the NanoBRET signaling were quantified with the Paradigm (Molecular Devices).

| Example | IC₅₀ [nM] |
|---|---|
| 95 | 2923 |
| 96 | 455 |
| 128 | 12 |
| 198 | 5 |
| 206 | 590 |
| 22 | 57 |
| 222 | 47 |
| 227 | 6 |
| 403 | 11 |
| 466 | 1152 |
| 475 | 2003 |
| 479 | 3435 |
| 500 | 171 |
| 527 | 4464 |
| 556 | 340 |
| 591 | 35 |
| 672 | 43 |
| 714 | 133 |
| 740 | 447 |
| 744 | 90 |
| 800 | 21 |
| 812 | 233 |

In one aspect, the present invention provides compounds of formula (I) and their pharmaceutically acceptable salts or esters as described herein, wherein said compounds of formula (I) and their pharmaceutically acceptable salts or esters have IC₅₀'s for MAGL NanoBRET target engagement below 25 μM, preferably below 10 μM, more preferably below 5 μM as measured in the MAGL assay described herein.

In one embodiment, compounds of formula (I) and their pharmaceutically acceptable salts or esters as described herein have IC₅₀ (MAGL inhibition) values between 0.0001 μM and 25 μM, particular compounds have IC₅₀ values between 0.0005 μM and 10 μM, further particular compounds have IC₅₀ values between 0.001 μM and 5 μM, as measured in the MAGL assay described herein.

### Using the Compounds of the Invention

In one aspect, the present invention provides compounds of formula (I), or pharmaceutically acceptable salts thereof, as described herein for use as therapeutically active substance.

In a further aspect, the present invention provides the use of compounds of formula (I), or pharmaceutically acceptable salts thereof, as described herein for the treatment or prophylaxis of neuroinflammation, neurodegenerative diseases, pain, cancer, mental disorders and/or inflammatory bowel disease in a mammal.

In one embodiment, the present invention provides the use of compounds of formula (I), or pharmaceutically acceptable salts thereof, as described herein for the treatment or prophylaxis of neuroinflammation and/or neurodegenerative diseases in a mammal.

In one embodiment, the present invention provides the use of compounds of formula (I), or pharmaceutically acceptable salts thereof, as described herein for the treatment or prophylaxis of neurodegenerative diseases in a mammal.

In one embodiment, the present invention provides the use of compounds of formula (I), or pharmaceutically acceptable salts thereof, as described herein for the treatment or prophylaxis of cancer in a mammal.

In one embodiment, the present invention provides the use of compounds of formula (I), or pharmaceutically acceptable salts thereof, as described herein for the treatment or prophylaxis of inflammatory bowel disease in a mammal.

In one embodiment, the present invention provides the use of compounds of formula (I), or pharmaceutically acceptable salts thereof, as described herein for the treatment or prophylaxis of pain in a mammal.

In one aspect, the present invention provides the use of compounds of formula (I), or pharmaceutically acceptable salts thereof, as described herein for the treatment or prophylaxis of multiple sclerosis, Alzheimer's disease, Parkinson's disease, amyotrophic lateral sclerosis, traumatic brain injury, neurotoxicity, stroke, epilepsy, anxiety, migraine, depression, hepatocellular carcinoma, colon carcinogenesis, ovarian cancer, neuropathic pain, chemotherapy induced neuropathy, acute pain, chronic pain, spasticity associated with pain, abdominal pain, abdominal pain associated with irritable bowel syndrome and/or visceral pain in a mammal.

In a preferred embodiment, the present invention provides the use of compounds of formula (I), or pharmaceutically acceptable salts thereof, as described herein for the treatment or prophylaxis of multiple sclerosis, Alzheimer's disease and/or Parkinson's disease in a mammal.

In a particularly preferred embodiment, the present invention provides the use of compounds of formula (I), or pharmaceutically acceptable salts thereof, as described herein for the treatment or prophylaxis of multiple sclerosis in a mammal.

In one aspect, the present invention provides compounds of formula (I), or pharmaceutically acceptable salts thereof, as described herein for use in the treatment or prophylaxis of neuroinflammation, neurodegenerative diseases, pain, cancer, mental disorders and/or inflammatory bowel disease in a mammal.

In one embodiment, the present invention provides compounds of formula (I), or pharmaceutically acceptable salts thereof, as described herein for use in the treatment or prophylaxis of neuroinflammation and/or neurodegenerative diseases in a mammal.

In one embodiment, the present invention provides compounds of formula (I), or pharmaceutically acceptable salts thereof, as described herein for use in the treatment or prophylaxis of cancer in a mammal.

In one embodiment, the present invention provides compounds of formula (I), or pharmaceutically acceptable salts thereof, as described herein for use in the treatment or prophylaxis of neurodegenerative diseases in a mammal.

In one embodiment, the present invention provides compounds of formula (I), or pharmaceutically acceptable salts thereof, as described herein for use in the treatment or prophylaxis of inflammatory bowel disease in a mammal.

In one embodiment, the present invention provides compounds of formula (I), or pharmaceutically acceptable salts thereof, as described herein for use in the treatment or prophylaxis of pain in a mammal.

In one aspect, the present invention provides compounds of formula (I), or pharmaceutically acceptable salts thereof, as described herein for use in the treatment or prophylaxis of multiple sclerosis, Alzheimer's disease, Parkinson's disease, amyotrophic lateral sclerosis, traumatic brain injury, neurotoxicity, stroke, epilepsy, anxiety, migraine, depression, hepatocellular carcinoma, colon carcinogenesis, ovarian cancer, neuropathic pain, chemotherapy induced neuropathy, acute pain, chronic pain, spasticity associated with pain, abdominal pain, abdominal pain associated with irritable bowel syndrome and/or visceral pain in a mammal.

In a preferred embodiment, the present invention provides compounds of formula (I), or pharmaceutically acceptable salts thereof, as described herein for use in the treatment or prophylaxis of multiple sclerosis, Alzheimer's disease and/or Parkinson's disease in a mammal.

In a particularly preferred embodiment, the present invention provides compounds of formula (I), or pharmaceutically acceptable salts thereof, as described herein for use in the treatment or prophylaxis of multiple sclerosis in a mammal.

In one aspect, the present invention provides the use of compounds of formula (I), or pharmaceutically acceptable salts thereof, as described herein for the preparation of a medicament for the treatment or prophylaxis of neuroinflammation, neurodegenerative diseases, pain, cancer, mental disorders and/or inflammatory bowel disease in a mammal.

In one embodiment, the present invention provides the use of compounds of formula (I), or pharmaceutically acceptable salts thereof, as described herein for the preparation of a medicament for the treatment or prophylaxis of neuroinflammation and/or neurodegenerative diseases in a mammal.

In one embodiment, the present invention provides the use of compounds of formula (I), or pharmaceutically acceptable salts thereof, as described herein for the preparation of a medicament for the treatment or prophylaxis of neurodegenerative diseases in a mammal.

In one embodiment, the present invention provides the use of compounds of formula (I), or pharmaceutically acceptable salts thereof, as described herein for the preparation of a medicament for the treatment or prophylaxis of cancer in a mammal.

In one embodiment, the present invention provides the use of compounds of formula (I), or pharmaceutically acceptable salts thereof, as described herein for the preparation of a medicament for the treatment or prophylaxis of inflammatory bowel disease in a mammal.

In one embodiment, the present invention provides the use of compounds of formula (I), or pharmaceutically acceptable salts thereof, as described herein for the preparation of a medicament for the treatment or prophylaxis of pain in a mammal.

In a further aspect, the present invention provides the use of compounds of formula (I), or pharmaceutically acceptable salts thereof, as described herein for the preparation of a medicament for the treatment or prophylaxis of multiple sclerosis, Alzheimer's disease, Parkinson's disease, amyotrophic lateral sclerosis, traumatic brain injury, neurotoxicity, stroke, epilepsy, anxiety, migraine, depression, hepatocellular carcinoma, colon carcinogenesis, ovarian cancer, neuropathic pain, chemotherapy induced neuropathy, acute pain, chronic pain, spasticity associated with pain, abdominal pain, abdominal pain associated with irritable bowel syndrome and/or visceral pain in a mammal.

In a preferred embodiment, the present invention provides the use of compounds of formula (I), or pharmaceutically acceptable salts thereof, as described herein for the preparation of a medicament for the treatment or prophylaxis of multiple sclerosis, Alzheimer's disease and/or Parkinson's disease in a mammal.

In a particularly preferred embodiment, the present invention provides the use of compounds of formula (I), or pharmaceutically acceptable salts thereof, as described herein for the preparation of a medicament for the treatment or prophylaxis of multiple sclerosis in a mammal.

In one aspect, the present invention provides a method for the treatment or prophylaxis of neuroinflammation, neurodegenerative diseases, pain, cancer, mental disorders and/or inflammatory bowel disease in a mammal, which method comprises administering an effective amount of a compound of formula (I), or a pharmaceutically acceptable salt thereof, as described herein to the mammal.

In one embodiment, the present invention provides a method for the treatment or prophylaxis of neuroinflammation and/or neurodegenerative diseases in a mammal, which method comprises administering an effective amount of a compound of formula (I), or a pharmaceutically acceptable salt thereof, as described herein to the mammal.

In one embodiment, the present invention provides a method for the treatment or prophylaxis of neurodegenerative diseases in a mammal, which method comprises administering an effective amount of a compound of formula (I), or a pharmaceutically acceptable salt thereof, as described herein to the mammal.

In one embodiment, the present invention provides a method for the treatment or prophylaxis of cancer in a mammal, which method comprises administering an effective amount of a compound of formula (I), or a pharmaceutically acceptable salt thereof, as described herein to the mammal.

In one embodiment, the present invention provides a method for the treatment or prophylaxis of inflammatory bowel disease in a mammal, which method comprises administering an effective amount of a compound of formula (I), or a pharmaceutically acceptable salt thereof, as described herein to the mammal.

In one embodiment, the present invention provides a method for the treatment or prophylaxis of pain in a mammal, which method comprises administering an effective amount of a compound of formula (I), or a pharmaceutically acceptable salt thereof, as described herein to the mammal.

In a further aspect, the present invention provides a method for the treatment or prophylaxis of multiple sclerosis, Alzheimer's disease, Parkinson's disease, amyotrophic lateral sclerosis, traumatic brain injury, neurotoxicity, stroke, epilepsy, anxiety, migraine, depression, hepatocellular carcinoma, colon carcinogenesis, ovarian cancer, neuropathic pain, chemotherapy induced neuropathy, acute pain, chronic pain, spasticity associated with pain, abdominal pain, abdominal pain associated with irritable bowel syndrome and/or visceral pain in a mammal, which method comprises administering an effective amount of a compound of formula (I), or a pharmaceutically acceptable salt thereof, as described herein to the mammal.

In a preferred embodiment, the present invention provides a method for the treatment or prophylaxis of multiple sclerosis, Alzheimer's disease and/or Parkinson's disease in a mammal, which method comprises administering an effective amount of a compound of formula (I), or a pharmaceutically acceptable salt thereof, as described herein to the mammal.

In a particularly preferred embodiment, the present invention provides a method for the treatment or prophylaxis of multiple sclerosis in a mammal, which method comprises administering an effective amount of a compound of formula (I), or a pharmaceutically acceptable salt thereof, as described herein to the mammal.

### Pharmaceutical Compositions and Administration

In one aspect, the present invention provides a pharmaceutical composition comprising a compound of formula (I) as described herein and a therapeutically inert carrier.

In one embodiment, the present invention provides the pharmaceutical compositions disclosed in Examples 866 and 867.

The compounds of formula (I) and their pharmaceutically acceptable salts and esters can be used as medicaments (e.g. in the form of pharmaceutical preparations). The pharmaceutical preparations can be administered internally, such as orally (e.g. in the form of tablets, coated tablets, dragées, hard and soft gelatin capsules, solutions, emulsions or suspensions), nasally (e.g. in the form of nasal sprays) or rectally (e.g. in the form of suppositories). However, the administration can also be effected parentally, such as intramuscularly or intravenously (e.g. in the form of injection solutions).

The compounds of formula (I) and their pharmaceutically acceptable salts and esters can be processed with pharmaceutically inert, inorganic or organic adjuvants for the production of tablets, coated tablets, dragées and hard gelatin capsules. Lactose, corn starch or derivatives thereof, talc, stearic acid or its salts etc. can be used, for example, as such adjuvants for tablets, dragées and hard gelatin capsules.

Suitable adjuvants for soft gelatin capsules are, for example, vegetable oils, waxes, fats, semi-solid substances and liquid polyols, etc.

Suitable adjuvants for the production of solutions and syrups are, for example, water, polyols, saccharose, invert sugar, glucose, etc.

Suitable adjuvants for injection solutions are, for example, water, alcohols, polyols, glycerol, vegetable oils, etc.

Suitable adjuvants for suppositories are, for example, natural or hardened oils, waxes, fats, semi-solid or liquid polyols, etc.

Moreover, the pharmaceutical preparations can contain preservatives, solubilizers, viscosity-increasing substances, stabilizers, wetting agents, emulsifiers, sweeteners, colorants, flavorants, salts for varying the osmotic pressure, buffers, masking agents or antioxidants. They can also contain still other therapeutically valuable substances.

The dosage can vary in wide limits and will, of course, be fitted to the individual requirements in each particular case. In general, in the case of oral administration a daily dosage of about 0.1 mg to 20 mg per kg body weight, preferably about 0.5 mg to 4 mg per kg body weight (e.g. about 300 mg per person), divided into preferably 1-3 individual doses, which can consist, for example, of the same amounts, should be appropriate. It will, however, be clear that the upper limit given herein can be exceeded when this is shown to be indicated.

### EXAMPLES

The invention will be more fully understood by reference to the following examples. The claims should not, however, be construed as limited to the scope of the examples.

In case the preparative examples are obtained as a mixture of enantiomers, the pure enantiomers can be separated by methods described herein or by methods known to the man skilled in the art, such as e.g., chiral chromatography (e.g., chiral SFC) or crystallization.

All reaction examples and intermediates were prepared under an argon atmosphere if not specified otherwise.

### Separation Methods

### HPLC Method GN

Gemini NX, 12 nm, 5 μm, 100×30 mm, 15 min gradient, ACN/Water+0.1% FA

### HPLC Method YMC

YMC-Triart C18, 12 nm, 5 μm, 100×30 mm, gradient, ACN/Water+0.1% FA

### Chiral Separation Methods

### HPLC Method OD1

Chiralcel OD, Normal Phase (isocratic): 80% Heptane; 20% Ethanol+NH₄Oac

### HPLC Method OD2

Chiralcel OD, Normal Phase (isocratic): 60% Heptane; 40% Ethanol+NH4Oac

### HPLC Method AD

Chiralpak-AD, Normal Phase (isocratic): 60% Heptane; 40% Ethanol+NH4Oac

### HPLC Method AD1

Chiralpak-AD, Normal Phase (isocratic): 70% Heptane; 30% Ethanol+NH4Oac

### HPLC Method NR

Reprosil Chiral NR, Normal Phase (isocratic): 60% Heptane; 40% Ethanol+NH4Oac

### SFC Method AY

(AY-H, 12 nm, 5 μm, 250×4.6 mm column), eluent: 30% MeOH in supercritical CO₂

### SFC Method ADH

(AD-H, 12 nm, 5 μm, 250×4.6 mm), eluent: 40% MeOH in supercritical CO₂

### SFC Method ADH1

(AD-H, 12 nm, 5 μm, 250×4.6 mm), eluent: 20% MeOH in supercritical CO₂

### SFC Method ADH2

(AD-H, 12 nm, 5 μm, 250×4.6 mm), eluent: 30% IPA (0.1% NH₃·H₂O) in supercritical CO2

SFC Method ADH3 (AD-H, 12 nm, 5 μm, 250×4.6 mm), eluent: 40% MeOH (0.1% NH3·H2O) in supercritical CO2

### SFC Method IC

(IC, 12 nm, 5 μm, 250×20 mm), eluent: 25% EtOH in supercritical CO2

### SFC Method IC1

(IC, 12 nm, 5 μm, 250×20 mm), eluent: 35% EtOH in supercritical CO2

### SFC Method IA

IA, 12 nm, 5 μm, 250×20 mm, 20% EtOH in supercritical CO2

### SFC Method IG

(IG, 12 nm, 5 μm, 250×20 mm), eluent: 40% MeOH (0.1% NH₃.H₂O) in supercritical CO2

### SFC Method IH

(IH, 5 μm, 250×20 mm), eluent: 35% MeOH in supercritical CO2

### SFC Method OJH

OJ-H, 12 nm, 5 μm, 250×20 mm. 15% EtOH in supercritical CO2

### SFC Method OJH1

OJ-H, 12 nm, 5 μm, 250×20 mm. 20% EtOH in supercritical CO2

### SFC Method OJH2

OJ-H, 12 nm, 5 μm, 250×20 mm. 25% IPA in supercritical CO2

### SFC Method ODH

OD-H, 12 nm, 5 μm, 250×4.6 mm. 20-40% EtOH in supercritical CO2

### SFC Method ODH1

OD-H, 5 nm, 5 μm, 250×20 mm. 30% MeOH in supercritical CO2

### SFC Method OD

Chiralcel OD, 250 mm*30 mm, 10 μm, 40% IPA (0.1% NH₃.H₂O) in supercritical CO2

### SFC Method OD1

Chiralcel OD, 250 mm*30 mm, 10 μm, 55% IPA (0.1% NH₃·H₂O) in supercritical CO2

### SFC Method OD3

Chiralcel OD, 250 mm*30 mm, 10 μm, 55% EtOH (0.1% NH₃.H₂O) in supercritical CO2

### SFC Method OD4

Chiralcel OD, 250 mm*30 mm, 10 μm, 50% IPA (0.1% NH₃·H₂O) in supercritical CO2

### SFC Method OD5

Chiralcel OD, 250 mm*30 mm, 10 μm, 45% MeOH (0.1% NH3·H2O) in supercritical CO2

### SFC Method AD

Chiralpak AD, 250 mm*30 mm, 10 μm, 40% (IPA/ACN 4/1)(0.1% NH3·H2O) in supercritical CO2

### SFC Method AD1

Chiralpak AD, 250 mm*30 mm, 10 μm, 50% (IPA/ACN 4/1)(0.1% NH3·H2O) in supercritical CO2

### SFC Method AD2

Chiralpak AD, 250 mm*30 mm, 10 μm, 55% EtOH (0.1% NH3·H2O) in supercritical CO2

### SFC Method AD3

Chiralpak AD, 250 mm*30 mm, 10 μm, 45% (IPA/ACN 4/1)(0.1% NH3·H2O) in supercritical CO2

### SFC Method AD4

Chiralpak AD, 250 mm*30 mm, 10 μm, 30% EtOH in supercritical CO2

### SFC Method AD5

Chiralpak AD, 250 mm*30 mm, 10 μm, 60% (MeOH/ACN 3/1)(0.1% NH3·H2O) in supercritical CO2

### SFC Method AD6

Chiralpak AD, 250 mm*30 mm, 10 μm, 30% MeOH (0.1% NH₃·H2O) in supercritical CO2

### SFC Method AD7

Chiralpak AD, 250 mm*30 mm, 10 μm, 50% MeOH (0.1% NH₃·H₂O) in supercritical CO2

### SFC Method CE

Cellulose-4 20×350 mm 5 μm, 35% MeOH (0.2% DEA) in supercritical CO2

### Example 1

### [3-[[2-Fluoro-6-(trifluoromethyl)phenyl]methoxy]azetidin-1-yl]-[3-(1H-pyrazol-5-yl)pyrrolidin-1-yl]methanone

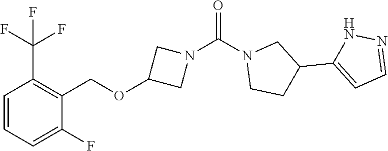 [SMILES: O=C(N1CC(OCc2c(F)cccc2C(F)(F)F)C1)N1CCC(c2ccn[nH]2)C1]

To an ice-cold suspension of bis(trichloromethyl) carbonate (80 mg, 270 μmol, CAS RN 32315 October 9) and NaHCO₃ (118 mg, 1.4 mmol) in DCM (1.5 mL) was added a solution of 3-((2-fluoro-6-(trifluoromethyl)benzyl)oxy)azetidine hydrochloride (100 mg, 350 μmol, CAS RN 1121613 July 7) in DCM (1.5 mL) and the mixture was stirred at RT overnight. To the suspension was added 5-(pyrrolidin-3-yl)-1H-pyrazole dihydrochloride (48 mg, 228 μmol, CAS RN 2173135-18-5) and DIPEA (313 μL, 1.79 mmol). The suspension was stirred at RT for 3.5 hours. The reaction mixture was poured on water and DCM and the layers were separated. The aqueous layer was extracted three times with DCM. The organic layers were dried over MgSO₄, filtered, and evaporated. The compound was purified using HPLC Method YMC to get the desired compound as a colorless gum (25.8 mg; 17.9%). MS (ESI): m/z=413.3 [M+H]⁺.

If not indicated otherwise the following examples were synthesized in analogy to the synthesis described for Example 1 using suitable building blocks, respectively.

| Ex. | Systematic Name | Structure | Building blocks | MS m/z |
|---|---|---|---|---|
| 2 | [3-[[2-Fluoro-4- (trifluoromethyl) phenyl]methoxy] azetidin-1-yl]-[3- (1H-pyrazol-5- yl)pyrrolidin-1- yl]methanone | 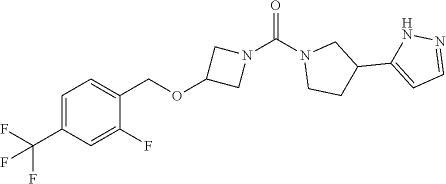 [SMILES: O=C(N1CC(OCc2ccc(C(F)(F)F)cc2F)C1)N1CCC(c2ccn[nH]2)C1] | 3-((2-Fluoro-4- (trifluoromethyl) benzyl)oxy)azetidine 4- methylbenzenesulfonic acid (CAS RN 2135785-94-1) and 5-(Pyrrolidin-3-yl)- 1H-pyrazole dihydrochloride (CAS RN 2173135-18-5) | 413.3 [M + H]⁺ |
| 3 | 4-[1-[3-[[2-Fluoro- 4-(trifluoromethyl) phenyl]methoxy] azetidine-1- carbonyl]pyrrolidin- 3-yl]oxazolidin-2- one | 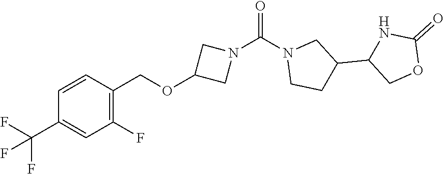 [SMILES: O=C1NC(C2CCN(C(=O)N3CC(OCc4ccc(C(F)(F)F)cc4F)C3)C2)CO1] | 3-((2-Fluoro-4- (trifluoromethyl) benzyl)oxy)azetidine 4-methylbenzenesulfonic acid (CAS RN 2135785-94-1) and 4-Pyrrolidin-3- yloxazolidin-2-one (CAS RN1888977-61-4) | 432.3 [M + H]⁺ |
| 9 | [4-[(4-Chloro-2- fluoro- phenoxy)methyl]- 1-piperidyl]-[3- (1H-pyrazol-5- yl)pyrrolidin-1- yl]methanone | 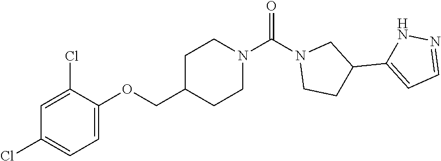 [SMILES: O=C(N1CCC(COc2ccc(Cl)cc2Cl)CC1)N1CCC(c2ccn[nH]2)C1] | 4-[(2-Chloro-4- fluorophenyl) methoxy]piperidine (CAS RN 1098356-25-2) and 5-(Pyrrolidin- 3-yl)-1H-pyrazole dihydrochloride (CAS RN 2173135-18-5) | 407.2 [M + H]⁺ |
| 10 | [3-[[2-Fluoro-4- (trifluoromethyl)phen yl]methoxy]azetidin- 1-yl]-[3-(1H-pyrazol- 5-yl)-1- piperidyl]methanone | 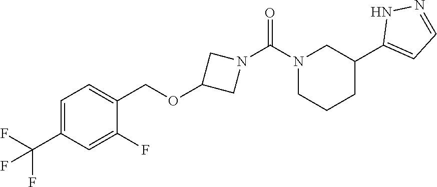 [SMILES: O=C(N1CC(OCc2ccc(C(F)(F)F)cc2F)C1)N1CCCC(c2ccn[nH]2)C1] | 3-((2-Fluoro-4- (trifluoromethyl) benzyl)oxy) azetidine4- methylbenzenesulfonic aci (CAS RN 2135785-94-1) and 3-(1H-Pyrazol-5- yl)piperidine (CAS RN 774479-26-4) | 427.2 [M + H]⁺ |
| 11 | [6-(2-Chloro-4- fluoro-phenoxy)-2- azaspiro[3.3]heptan- 2-yl]-[3-(1H-pyrazol- 5-yl)pyrrolidin-1- yl]methanone | 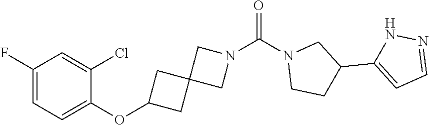 [SMILES: O=C(N1CCC(c2ccn[nH]2)C1)N1CC2(CC(Oc3ccc(F)cc3Cl)C2)C1] | 6-(2-Chloro-4- fluoro-phenoxy)-2- azaspiro[3.3]heptane 2,2,2- trifluoroacetic acid (BB1) and 5- (Pyrrolidin-3-yl)- 1H-pyrazole dihydrochloride (CAS RN 2173135-18-5) | 405.2 [M + H]⁺ |
| 12 | [3-(1H-Pyrazol-5- yl)pyrrolidin-1-yl]- [4-[[4- (trifluoromethyl) phenyl]methyl]-1- piperidyl]methanone | 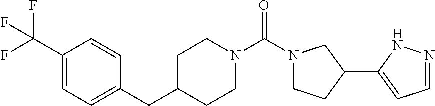 [SMILES: O=C(N1CCC(Cc2ccc(C(F)(F)F)cc2)CC1)N1CCC(c2ccn[nH]2)C1] | 4-[[4-(Trifluoromethyl) phenyl]methyl] piperidine hydrochloride (CAS RN 193357-81-2) and 5-(Pyrrolidin-3-yl)- 1H-pyrazole dihydrochloride (CAS RN 2173135-18-5) | 407.3 [M + H]⁺ |
| 14 | [3-[[2-Fluoro-4- (pentafluoro-26- sulfanyl)phenyl] methoxy]azetidin- 1-yl]-[3-(1H-pyrazol- 5-yl)pyrrolidin-1- yl]methanone | 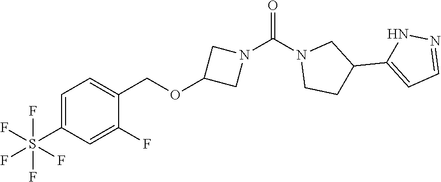 [SMILES: O=C(N1CC(OCc2ccc(S(F)(F)(F)(F)F)cc2F)C1)N1CCC(c2ccn[nH]2)C1] | 3-((2-Fluoro-4- (pentafluoro- 26- sulfaneyl)benzyl) oxy)azetidine 2,2,2- trifluoroacetic acid (BB2) and 5- (Pyrrolidin-3-yl)- 1H-pyrazole dihydrochloride (CAS RN 2173135-18-5) | 471.2 [M + H]⁺ |
| 18 | [3-[[2-Fluoro-4- (trifluoromethyl) phenyl]methoxy] azetidin- 1-yl]-[3-(1H-1,2,4- triazol-5- yl)pyrrolidin-1- yl]methanone | 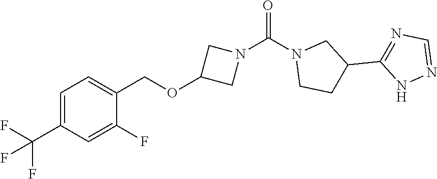 [SMILES: O=C(N1CC(OCc2ccc(C(F)(F)F)cc2F)C1)N1CCC(c2ncn[nH]2)C1] | 3-((2-Fluoro-4- (trifluoromethyl)be nzyl)oxy)azetidine 4- methylbenzenesulfonic acid (CAS RN 2135785-94-1) and 3-Pyrrolidin-3-yl- 4H-1,2,4-triazole hydrochloride (CAS RN 1909337-56-9) | 414.2 [M + H]⁺ |
| 19 | 6-[1-[3-[[2-Fluoro-4- (trifluoromethyl) phenyl]methoxy] azetidine-1- carbonyl]pyrrolidin- 3-yl]-1H-pyridin-2- one | 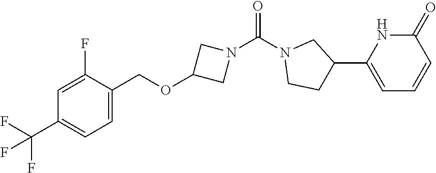 [SMILES: O=C(N1CC(OCc2ccc(C(F)(F)F)cc2F)C1)N1CCC(c2cccc(=O)[nH]2)C1] | 3-((2-Fluoro-4- (trifluoromethyl)be nzyl)oxy)azetidine 4- methylbenzenesulfonic acid (CAS RN 2135785-94-1) and 6-Pyrrolidin-3-yl- 1H-pyridin-2-one (CAS RN 1260817-38-6) | 440.2 [M + H]⁺ |
| 20 | 6-[1-[3-[[2-Fluoro-4- (trifluoromethyl) phenyl]methoxy] azetidine-1- carbonyl]pyrrolidin- 3-yl]-1H-pyrazin-2- one | 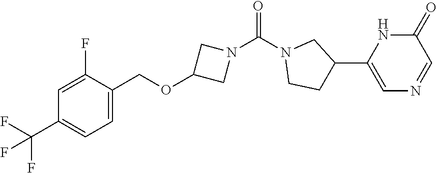 [SMILES: O=C(N1CC(OCc2ccc(C(F)(F)F)cc2F)C1)N1CCC(c2cncc(=O)[nH]2)C1] | 3-((2-Fluoro-4- (trifluoromethyl)be nzyl)oxy)azetidine 4- methylbenzenesulfonic acid (CAS RN 2135785-94-1) and 6-Pyrrolidin-3-yl- 1H-pyrazin-2-one hydrobromide (BB4) | 441.2 [M + H]⁺ |
| 21 | [3-[[2-Fluoro-4- (trifluoromethyl) phenyl] methoxy]azetidin- 1-yl]-[3-(4-isopropyl- 1H-pyrazol-3- yl)pyrrolidin-1- yl]methanone | 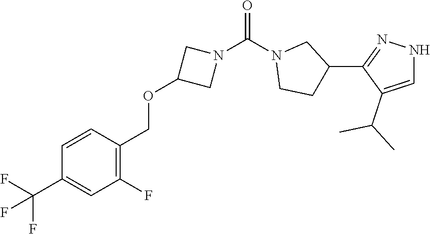 [SMILES: CC(C)c1c[nH]nc1C1CCN(C(=O)N2CC(OCc3ccc(C(F)(F)F)cc3F)C2)C1] | 3-((2-Fluoro-4- (trifluoromethyl)be nzyl)oxy)azetidine 4- methylbenzenesulfonic acid (CAS RN 2135785-94-1) and 4-Isopropyl-3- pyrrolidin-3-yl-1H- pyrazole (CAS RN 1369158-51-9) | 455.2 [M + H]⁺ |
| 26 | Ethyl 5-[1-[3-[[2- fluoro-4- (trifluoromethyl) phenyl]methoxy] azetidine-1- carbonyl]pyrrolidin- 3-yl]-1H-pyrazole-4- carboxylate | 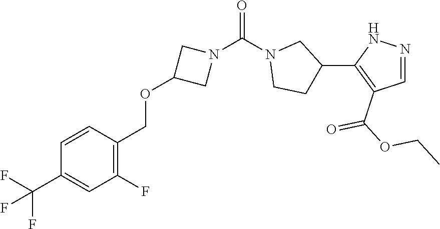 [SMILES: CCOC(=O)c1cn[nH]c1C1CCN(C(=O)N2CC(OCc3ccc(C(F)(F)F)cc3F)C2)C1] | 3-((2-Fluoro-4- (trifluoromethyl) benzyl)oxy)azetidine 4- methylbenzenesulfonic acid (CAS RN 2135785-94-1) and Ethyl 5-pyrrolidin- 3-yl-1H-pyrazole- 4-carboxylate dihydrochloride (supplier: Enamine, MDL # MFCD30181333) | 485.2 [M + H]⁺ |
| 27 | 5-[1-[3-[[2-Fluoro-4- (trifluoromethyl) phenyl]methoxy] azetidine-1- carbonyl]pyrrolidin- 3-yl]pyrrolidin-2-one | 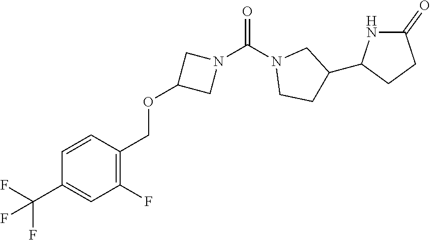 [SMILES: O=C1CCC(C2CCN(C(=O)N3CC(OCc4ccc(C(F)(F)F)cc4F)C3)C2)N1] | 3-((2-Fluoro-4- (trifluoromethyl) benzyl)oxy)azetidine 4- methylbenzenesulfonic acid (CAS RN 2135785-94-1) and 5-Pyrrolidin-3- ylpyrrolidin-2-one 4- methylbenzenesulfonic acid (BB7) [ENTITY A] | 430.2 [M + H]⁺ |
| 28 | 5-[1-[3-[[2-Fluoro-4- (trifluoromethyl) phenyl]methoxy] azetidine-1- carbonyl]pyrrolidin- 3-yl]pyrrolidin-2-one | 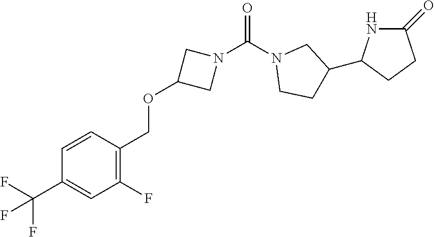 [SMILES: O=C1CCC(C2CCN(C(=O)N3CC(OCc4ccc(C(F)(F)F)cc4F)C3)C2)N1] | 3-((2-Fluoro-4- (trifluoromethyl) benzyl)oxy)azetidine 4- methylbenzenesulfonic acid (CAS RN 2135785-94-1) and 5-Pyrrolidin-3- ylpyrrolidin-2-one 4- methylbenzenesulfonic acid (BB8) [ENTITY B] | 430.2 [M + H]⁺ |
| 35 | 1-[3-[[2-Fluoro-4- (trifluoromethyl) phenyl]methoxy] azetidine-1- carbonyl]pyrrolidine- 3-carboxamide | 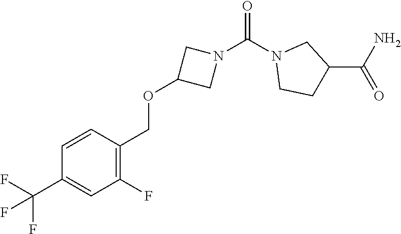 [SMILES: NC(=O)C1CCN(C(=O)N2CC(OCc3ccc(C(F)(F)F)cc3F)C2)C1] | 3-((2-Fluoro-4- (trifluoromethyl) benzyl)oxy)azetidine 4- methylbenzenesulfonic acid (CAS RN 2135785-94-1) and Pyrrolidine-3- carboxamide hydrochloride (CAS RN 644972-57-6) | 390.2 [M + H]⁺ |
| 37 | [3-(1H-1,2,4-Triazol- 5-yl)pyrrolidin-1-yl]- [3-[4-[1- (trifluoromethyl) cyclopropyl]phenyl] azetidin-1-yl] methanone | 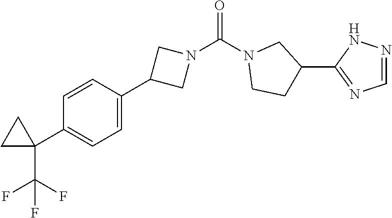 [SMILES: O=C(N1CC(c2ccc(C3(C(F)(F)F)CC3)cc2)C1)N1CCC(c2ncn[nH]2)C1] | 3-[4-[1- (Trifluoromethyl) cyclopropyl]phenyl] azetidine 4- methylbenzenesulfonic acid (BB9) and 3-Pyrrolidin-3- yl-4H-1,2,4-triazole hydrochloride (CAS RN 1909337-56-9) | 406.3 [M + H]⁺ |
| 38 | [3-(4-tert- Butylphenyl) azetidin- 1-yl]-[3-(1H-1,2,4- triazol-5- yl)pyrrolidin-1- yl]methanone | 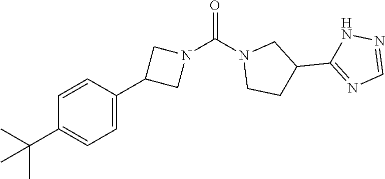 [SMILES: CC(C)(C)c1ccc(C2CN(C(=O)N3CCC(c4ncn[nH]4)C3)C2)cc1] | 3-(4-(tert- Butyl)phenyl)azetidine 4-methylbenzenesulfonic acid (CAS RN 2377009-52-2) and 3-Pyrrolidin-3-yl- 4H-1,2,4-triazole hydrochloride (CAS RN 1909337-56-9) | 354.3 [M + H]⁺ |
| 89 | [4-[bis(4- Fluorophenyl) methyl]- 1-piperidyl]-[3- (1H-1,2,4-triazol-5- yl)pyrrolidin-1- yl]methanone | 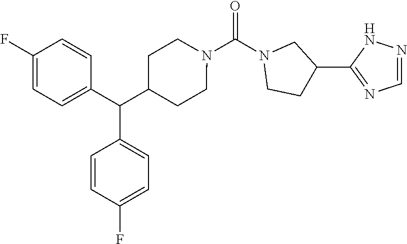 [SMILES: O=C(N1CCC(C(c2ccc(F)cc2)c2ccc(F)cc2)CC1)N1CCC(c2ncn[nH]2)C1] | 4-(bis(4- Fluorophenyl) methyl)piperidine (CAS RN 60285-00-9) and 3-Pyrrolidin-3- yl-4H-1,2,4-triazole hydrochloride (CAS RN 1909337-56-9) | 452.2 [M + H]⁺ |

### Example 4

### (+)- or (−)-[3-[[2-Fluoro-4-(trifluoromethyl)phenyl]methoxy]azetidin-1-yl]-[3-(1H-pyrazol-5-yl)pyrrolidin-1-yl]methanone and Example 5 (−)- or (+)-[3-[[2-Fluoro-4-(trifluoromethyl)phenyl]methoxy]azetidin-1-yl]-[3-(1H-pyrazol-5-yl)pyrrolidin-1-yl]methanone

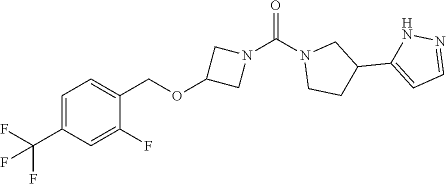 [SMILES: O=C(N1CC(OCc2ccc(C(F)(F)F)cc2F)C1)N1CCC(c2ccn[nH]2)C1]

The racemate of [3-[2-fluoro-4-(trifluoromethyl)phenyl]methoxy]azetidin-1-yl]-[3-(1H-pyrazol-5-yl)pyrrolidin-1-yl]methanone (136 mg, 0.33 mmol, example 1) was separated by HPLC Method OD1 to give the desired product 4 (first eluting) as a yellow oil (47 mg, 15.4%). MS (ESI): m/z=413.3 [M+H]⁺ and the desired product 5 (second eluting) as a yellow oil (37 mg, 14.6%). MS (ESI): m/z=413.3 [M+H]⁺.

If not indicated otherwise the following examples were separated in analogy to the synthesis described for Examples 4 and 5 using suitable starting material, respectively.

| | | | Starting | |
|---|---|---|---|---|
| | | | material | |
| | | | Chroma- | |
| | | | tography | |
| | | | parameters | |
| | Systematic | | Order | MS |
| Ex. | Name | Structure | of elution | m/z |
| 15 | (+)- or (−)-[3- [[2-Fluoro- 6-(trifluoro- methyl)phenyl] methoxy]aze- tidin-1-yl]-[3- (1H-pyrazol-5- yl)pyrrolidin-1- yl]methanone | 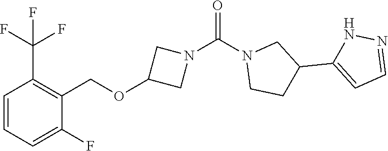 [SMILES: O=C(N1CC(OCc2c(F)cccc2C(F)(F)F)C1)N1CCC(c2ccn[nH]2)C1] | Example 1 SFC Method AY first eluting | 413.2 [M + H]⁺ |
| 16 | (−) or (+)-[3- [[2-Fluoro- 6-(tri- fluoromethyl) phenyl]meth- oxy]azeti- din-1-yl]-[3- (1H-pyrazol-5- yl)pyrrolidin-1- yl]methanone | 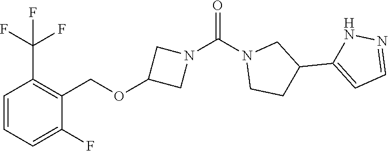 [SMILES: O=C(N1CC(OCc2c(F)cccc2C(F)(F)F)C1)N1CCC(c2ccn[nH]2)C1] | Example 1 SFC Method AY second eluting | 413.2 [M + H]⁺ |
| 22 | 4-[1-[3-[[2- Fluoro-4-(tri- fluoromet- hyl)phenyl] methoxy]azeti- dine-1-carbo- nyl]pyrrolidin- 3-yl]oxazoli- din-2-one | 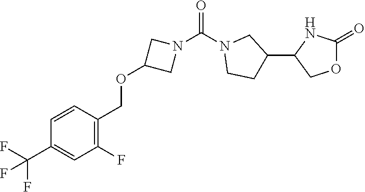 [SMILES: O=C1NC(C2CCN(C(=O)N3CC(OCc4ccc(C(F)(F)F)cc4F)C3)C2)CO1] | BB5 SFC Method ADH first eluting | 432.2 [M + H]⁺ |
| 23 | 4-[1-[3-[[2- Fluoro-4-(tri- fluoromethl) phenyl] methoxy]azeti- dine-1-carbo- nyl]pyrrolidin- 3-yl]oxazoli- din-2-one | 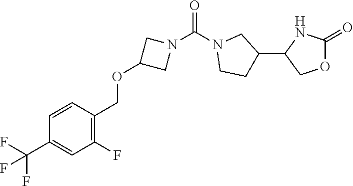 [SMILES: O=C1NC(C2CCN(C(=O)N3CC(OCc4ccc(C(F)(F)F)cc4F)C3)C2)CO1] | BB5 SFC Method ADH second eluting | 432.2 [M + H]⁺ |
| 24 | 4-[1-[3-[[2- Fluoro-4-(tri- fluorometh- yl)phenyl] methoxy]azeti- dine-1-carbo- nyl]pyrrolidin- 3-yl]oxazoli- din-2-one | 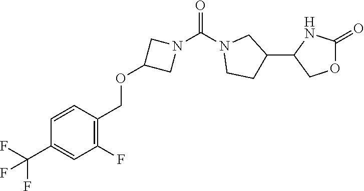 [SMILES: O=C1NC(C2CCN(C(=O)N3CC(OCc4ccc(C(F)(F)F)cc4F)C3)C2)CO1] | BB6 HPLC Method OD2 first eluting | 432.1 [M + H]⁺ |
| 25 | 4-[1-[3-[[2- Fluoro-4- (trifluorometh- yl)phenyl] methoxy]azeti- dine-1-carbo- nyl]pyrrolidin- 3-yl]oxazoli- din-2-one | 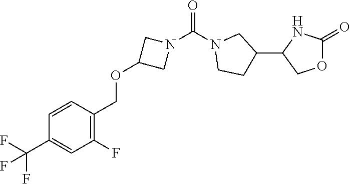 [SMILES: O=C1NC(C2CCN(C(=O)N3CC(OCc4ccc(C(F)(F)F)cc4F)C3)C2)CO1] | BB6 HPLC Method OD2 second eluting | 432.1 [M + H]⁺ |
| 29 | [3-[[2-Fluoro- 4-(trifluoro- methyl)phe- nyl]meth- oxy]azetidin- 1-yl]-[(3R)-3- (1H-1,2,4- triazol-5-yl) pyrrolidin-1- | 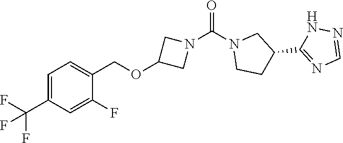 [SMILES: O=C(N1CC(OCc2ccc(C(F)(F)F)cc2F)C1)N1CC[C@@H](c2ncn[nH]2)C1] | Example 18 HPLC Method AD first eluting | 414.2 [M + H]⁺ |
| | yl]methanone | | | |
| 30 | [3-[[2-Fluoro- 4-(trifluoro- methyl)phenyl] methoxy]azeti- din-1-yl]-[(3S)- 3-(1H-1,2,4- triazol-5-yl) pyrrolidin-1- yl]methanone | 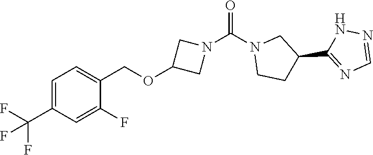 [SMILES: O=C(N1CC(OCc2ccc(C(F)(F)F)cc2F)C1)N1CC[C@H](c2ncn[nH]2)C1] | Example 18 HPLC Method AD second eluting | 414.2 [M + H]⁺ |
| 31 | 5-[1-[3-[[2- Fluoro-4-(tri- fluorometh- yl)phenyl] methoxy]aze- tidine-1-carbo- nyl]pyrrolidin- 3-yl]pyrrolidin- 2-one | 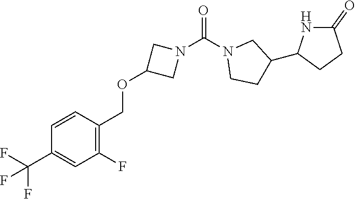 [SMILES: O=C1CCC(C2CCN(C(=O)N3CC(OCc4ccc(C(F)(F)F)cc4F)C3)C2)N1] | example 27 HPLC Method AD first eluting | 430.3 [M + H]⁺ |
| 32 | 5-[1-[3-[[2- Fluoro-4-(tri- fluorometh- yl)phenyl] methoxy]aze- tidine-1-carbo- nyl]pyrrolidin- 3-yl]pyrrolidin- 2-one | 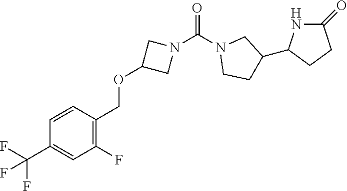 [SMILES: O=C1CCC(C2CCN(C(=O)N3CC(OCc4ccc(C(F)(F)F)cc4F)C3)C2)N1] | example 27 HPLC Method AD second eluting | 430.3 [M + H]⁺ |
| 33 | 5-[1-[3-[[2- Fluoro-4-(tri- fluorometh- yl)phenyl] methoxy]azeti- dine-1-carbo- nyl]pyrrolidin- 3-yl]pyrrolidin- 2-one | 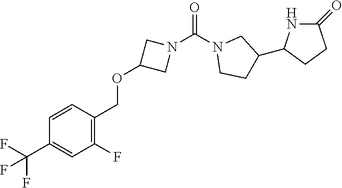 [SMILES: O=C1CCC(C2CCN(C(=O)N3CC(OCc4ccc(C(F)(F)F)cc4F)C3)C2)N1] | example 28 HPLC Method OD2 first eluting | 430.3 [M + H]⁺ |
| 34 | 5-[1-[3-[[2- Fluoro-4-(tri- fluorometh- yl)phenyl] methoxy]azeti- dine-1-carbo- nyl]pyrrolidin- 3-yl]pyrrolidin- 2-one | 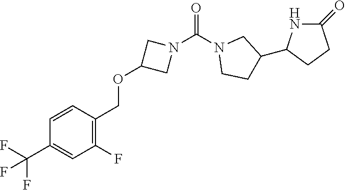 [SMILES: O=C1CCC(C2CCN(C(=O)N3CC(OCc4ccc(C(F)(F)F)cc4F)C3)C2)N1] | example 28 HPLC Method OD2 second eluting | 430.3 [M + H]⁺ |
| 44 | (+)- or (−)-[3- (1H-Pyrazol-5- yl)pyrrolidin-1- yl]-[3-(4-tetra- hydropyran-4- ylphenyl)aze tidin-1- yl]methanone | 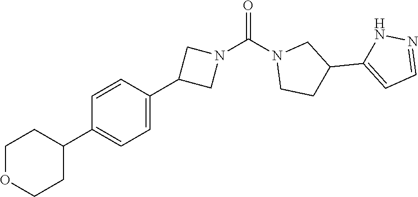 [SMILES: O=C(N1CC(c2ccc(C3CCOCC3)cc2)C1)N1CCC(c2ccn[nH]2)C1] | BB14 SFC Method AD1 first eluting | 381.2 [M + H]⁺ |
| 45 | (−)- or (+)- [3- (1H-Pyrazol-5- yl)pyrrolidin-1- yl]-[3-(4-tetra- hydropyran-4- ylphenyl)aze- tidin- 1-yl]methanone | 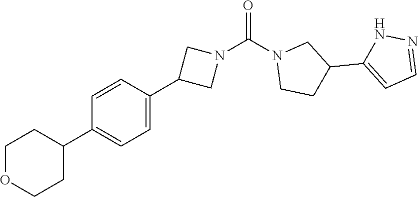 [SMILES: O=C(N1CC(c2ccc(C3CCOCC3)cc2)C1)N1CCC(c2ccn[nH]2)C1] | BB14 SFC Method AD1 second eluting | 381.2 [M + H]⁺ |
| 46 | (+)- or (−)-[3- (4-Isopropoxy- phenyl)aze- tidin-1-y1]-[3- (1H-pyrazol-5- yl)pyrrolidin-1- yl]methanone | 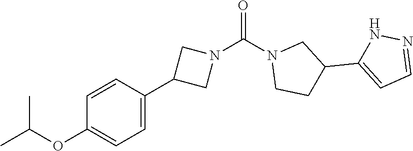 [SMILES: CC(C)Oc1ccc(C2CN(C(=O)N3CCC(c4ccn[nH]4)C3)C2)cc1] | BB15 SFC Method AD3 first eluting | 355.2 [M + H]⁺ |
| 47 | (−)- or (+)-[3- (4-Isopro- poxyphenyl) azetidin-1-yl]- [(3S)-3-(1H- pyrazol-5- yl)pyrrolidin-1- yl]methanone | 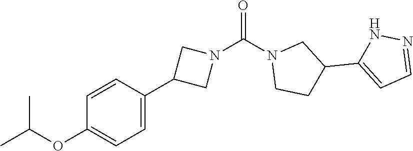 [SMILES: CC(C)Oc1ccc(C2CN(C(=O)N3CCC(c4ccn[nH]4)C3)C2)cc1] | BB15 SFC Method AD3 second eluting | 355.2 [M + H]⁺ |
| 54 | [3-[[2-Fluoro- 4-(trifluoro- methyl)phenyl] methoxy]azeti- din-1-yl]-[(3S)- 3-(1H-triazol- 5-yl)pyrrolidin- 1-yl]methanone | 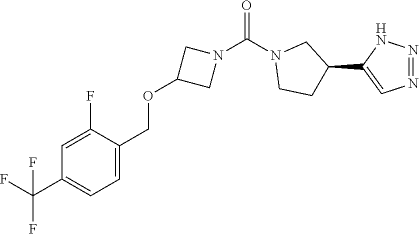 [SMILES: O=C(N1CC(OCc2ccc(C(F)(F)F)cc2F)C1)N1CC[C@H](c2cnn[nH]2)C1] | example 36, HPLC Method AD first eluting | 414.2 [M + H]⁺ |
| 55 | [3-[[2-Fluoro- 4-(trifluoro- methyl) phenyl] methoxy]azeti- din-1-yl]- [(3R)-3-(1H- triazol-5-yl) pyrrolidin-1- yl]methanone | 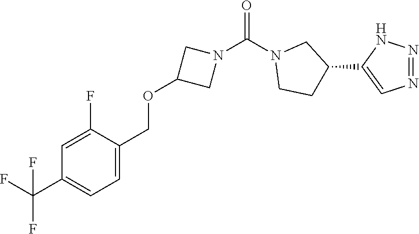 [SMILES: O=C(N1CC(OCc2ccc(C(F)(F)F)cc2F)C1)N1CC[C@@H](c2cnn[nH]2)C1] | example 36, HPLC Method AD second eluting | 414.3 [M + H]⁺ |
| 56 | (+)- or (−)-[3- [[2-Fluoro-4- (trifluorometh- yl)phenyl] methoxy]azeti- din-1-yl]-[3- imidazol-1- ylpyrrolidin-1- yl]methanone | 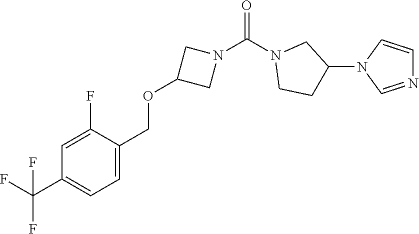 [SMILES: O=C(N1CC(OCc2ccc(C(F)(F)F)cc2F)C1)N1CCC(n2ccnc2)C1] | example 40, HPLC Method AD first eluting | 413.2 [M + H]⁺ |
| 57 | (−)- or (+)-[3- [[2-Fluoro-4- (trifluorometh- yl)phenyl] methoxy]azeti- din-1-yl]-[3- imidazol-1- ylpyrrolidin-1- yl]methanone | 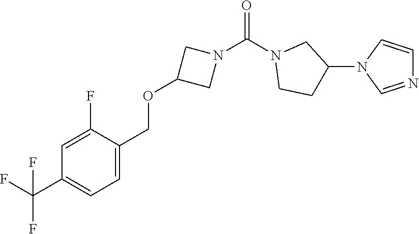 [SMILES: O=C(N1CC(OCc2ccc(C(F)(F)F)cc2F)C1)N1CCC(n2ccnc2)C1] | example 40, HPLC Method AD second eluting | 413.3 [M + H]⁺ |
| 62 | (+)- or (−)-[3- (4-Propoxy- phenyl)aze- tidin-1-yl]-[3- (1H-pyrazol-5- yl)pyrrolidin-1- yl]methanone | 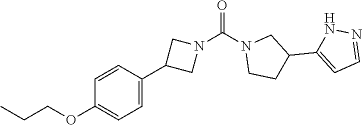 [SMILES: CCCOc1ccc(C2CN(C(=O)N3CCC(c4ccn[nH]4)C3)C2)cc1] | BB16 SFC Method AD2 first eluting | 355.1 [M + H]⁺ |
| 63 | (−)- or (+)-[3- (4-Propoxy- phenyl)aze- tidin-1-yl]-[3- (1H-pyrazol-5- yl)pyrrolidin-1- yl]methanone | 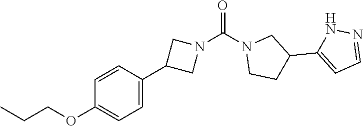 [SMILES: CCCOc1ccc(C2CN(C(=O)N3CCC(c4ccn[nH]4)C3)C2)cc1] | BB16 SFC Method AD2 second eluting | 355.1 [M + H]⁺ |
| 64 | (+)- or (−)-[3- [[2,4-bis(Tri- fluoromethyl) phenyl]metho- xy]azetidin- 1-yl]-[3-(1H- 1,2,4-triazol- 5-yl)pyrrolidin- 1-yl]methanone | 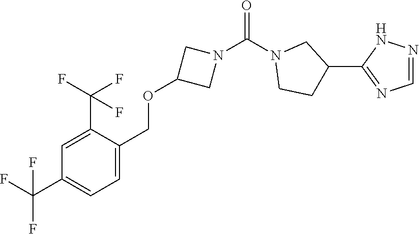 [SMILES: O=C(N1CC(OCc2ccc(C(F)(F)F)cc2C(F)(F)F)C1)N1CCC(c2ncn[nH]2)C1] | Example 41 HPLC Method AD1 first eluting | 464.3 [M + H]⁺ |
| 65 | (−)- or (+)-[3- [[2,4-bis(Tri- fluorometh- yl)phenyl] methoxy]aze- tidin-1-yl]-[3- (1H-1,2,4- triazol-5-yl) pyrrolidin-1- yl]methanone | 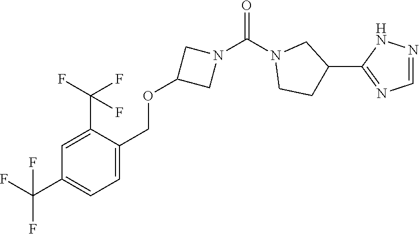 [SMILES: O=C(N1CC(OCc2ccc(C(F)(F)F)cc2C(F)(F)F)C1)N1CCC(c2ncn[nH]2)C1] | Example 41 HPLC Method AD1 second eluting | 464.3 [M + H]⁺ |
| 75 | [3-(1H- Pyrazol-5-yl) pyrrolidin- 1-yl]-[3- (4-THF-3-yl- phenyl)aze- tidin-1- yl]methanone | 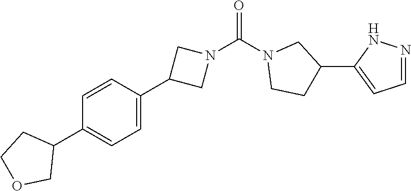 [SMILES: O=C(N1CC(c2ccc(C3CCOC3)cc2)C1)N1CCC(c2ccn[nH]2)C1] | BB17 SFC Method OD5 first eluting, mixture with inactive isomer followed by SFC Method AD1 first eluting | 367.2 [M + H]⁺ |
| 76 | [3-(1H-Pyra- zol-5- yl)pyrroli- din-1-yl]-[3- (4-THF-3-yl- phenyl)aze- tidin-1- yl]methanone | 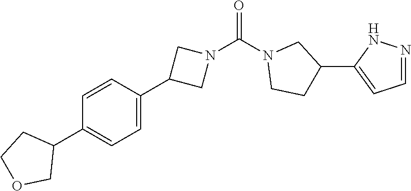 [SMILES: O=C(N1CC(c2ccc(C3CCOC3)cc2)C1)N1CCC(c2ccn[nH]2)C1] | BB17 SFC Method OD5 second eluting, mixture with example 77 followed by SFC Method AD1 first eluting | 367.2 [M + H]⁺ |
| 77 | [3-(1H- pyrazol- 5-yl)pyrro- lidin-1-yl]- [3-(4-THF-3- ylphenyl)aze- tidin-1-yl] methanone | 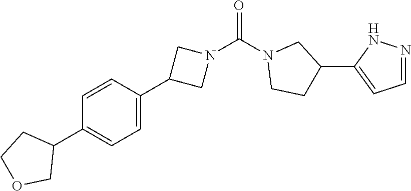 [SMILES: O=C(N1CC(c2ccc(C3CCOC3)cc2)C1)N1CCC(c2ccn[nH]2)C1] | BB17 SFC Method OD5 second eluting, mixture with example 76 followed by SFC Method AD1 second eluting | 367.2 [M + H]⁺ |
| 80 | [3-(1H- Pyrazol-5- yl)pyrrolidin- 1-yl]-[3-(4- tetrahydropy- ran-3-ylphe- nyl)azetidin- 1-yl]metha- none | 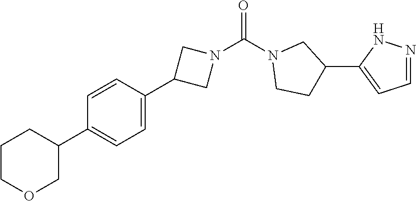 [SMILES: O=C(N1CC(c2ccc(C3CCCOC3)cc2)C1)N1CCC(c2ccn[nH]2)C1] | BB18 SFC Method OD4 second eluting, mixture with example 81 followed by SFC Method AD first eluting | 381.2 [M + H]⁺ |
| 81 | [3-(1H- Pyrazol-5- yl)pyrrolidin- 1-yl]-[3-(4- tetrahydro- pyran-3-ylphe- nyl)azetidin- 1-yl]metha- none | 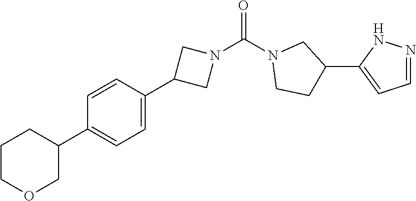 [SMILES: O=C(N1CC(c2ccc(C3CCCOC3)cc2)C1)N1CCC(c2ccn[nH]2)C1] | BB18 SFC Method OD4 second eluting, mixture with example 80 followed by SFC Method AD second eluting | 381.2 [M + H]⁺ |
| 84 | (+)- or (−)-[3- [4-[(1R,5S)- 8-Oxa-3-aza- bicyclo[3.2.1] octan-3-yl] phenyl]aze- tidin-1-yl]- [3-(1H-pyra- zol-5-yl) pyrrolidin-1- | 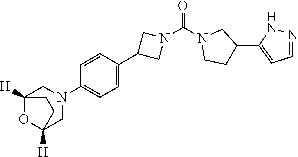 [SMILES: O=C(N1CC(c2ccc(N3C[C@H]4CC[C@@H](C3)O4)cc2)C1)N1CCC(c2ccn[nH]2)C1] | BB19 SFC Method OD3 first eluting | 408.2 [M + H]⁺ |
| | yl]methanone | | | |
| 85 | (−)- or (+)-[3- [4-[(1R,5S)- 8-Oxa- 3-azabicyclo [3.2.1]octan-3- yl]phenyl]azeti- din-1-yl]-[3- (1H-pyrazol-5- yl)pyrrolidin-1- yl]methanone | 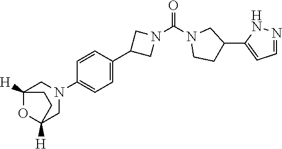 [SMILES: O=C(N1CC(c2ccc(N3C[C@H]4CC[C@@H](C3)O4)cc2)C1)N1CCC(c2ccn[nH]2)C1] | BB19 SFC Method OD3 second eluting | 408.2 [M + H]⁺ |
| 95 | (+)- or (−)-[3- (1H-1,2,4- Triazol-5- yl)pyrrolidin-1- yl]-[3-[4-[1- (trifluoro- methyl)cyclo- propyl]phe- nyl]azetidin-1- yl]methanone | 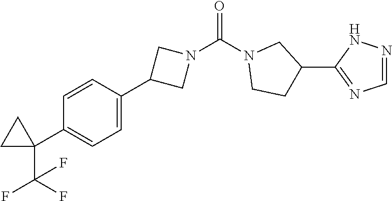 [SMILES: O=C(N1CC(c2ccc(C3(C(F)(F)F)CC3)cc2)C1)N1CCC(c2ncn[nH]2)C1] | Example 37 HPLC Method AD1 first eluting | 406.2 [M + H]⁺ |
| 96 | (−)- or (+)-[3- (1H-1,2,4- Triazol-5- yl)pyrrolidin-1- yl]-[3-[4-[1-(tri- fluorometh- yl)cyclo- propyl]phe- nyl]azetidin-1- yl]methanone | 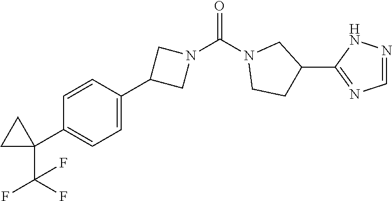 [SMILES: O=C(N1CC(c2ccc(C3(C(F)(F)F)CC3)cc2)C1)N1CCC(c2ncn[nH]2)C1] | Example 37 HPLC Method AD1 second eluting | 406.2 [M + H]⁺ |
| 97 | (+)- or (-)-[3- [[2-Chloro-4- (trifluorometh- yl)phenyl] methoxy]azeti- din-1-yl]-[3- (1H-1,2,4-tria- zol-5-yl)pyrro- lidin-1- yl]methanone | 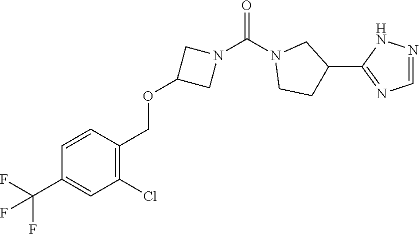 [SMILES: O=C(N1CC(OCc2ccc(C(F)(F)F)cc2Cl)C1)N1CCC(c2ncn[nH]2)C1] | Example 59 SFC Method AD4 first eluting | 430.2 [M + H]⁺ |
| 98 | (−)- or (+)-[3- [[2-Chloro- 4-(trifluoro- methyl)phe- nyl]meth- oxy]azeti- din-1-yl]-[3- (1H-1,2,4- triazol-5-yl) pyrrolidin-1- yl]methanone | 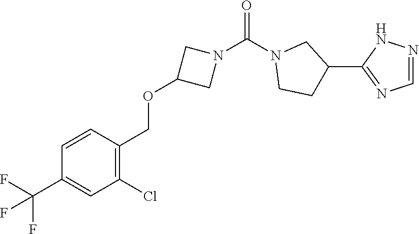 [SMILES: O=C(N1CC(OCc2ccc(C(F)(F)F)cc2Cl)C1)N1CCC(c2ncn[nH]2)C1] | Example 59 SFC Method AD4 second eluting | 430.2 [M + H]⁺ |
| 103 | (+)- or (−)-[3- [4-(Oxetan- 3-yl)phe- nyl]azetidin- 1-yl]-[3-(1H- pyrazol-5-yl) pyrrolidin-1- yl]methanone | 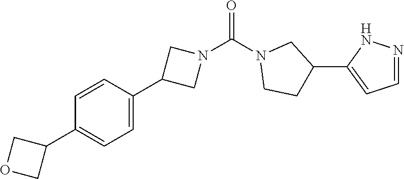 [SMILES: O=C(N1CC(c2ccc(C3COC3)cc2)C1)N1CCC(c2ccn[nH]2)C1] | Example 102 SFC Method OD1 first eluting | 353.1 [M + H]⁺ |
| 104 | (−)- or (+)-[3- [4-(Oxe- tan-3-yl)phe- nyl]azetidin- 1-yl]-[3-(1H- pyrazol-5-yl) pyrrolidin-1- yl]methanone | 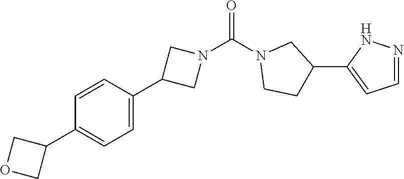 [SMILES: O=C(N1CC(c2ccc(C3COC3)cc2)C1)N1CCC(c2ccn[nH]2)C1] | Example 102 SFC Method OD1 second eluting | 353.1 [M + H]⁺ |
| 112 | (+)- or (−)-[3- (1H-Triazol-5- yl)pyrrolidin-1- yl]-[3-[4-[1- (trifluorometh- yl)cyclopro- pyl]phe- nyl]azetidin-1- yl]methanone | 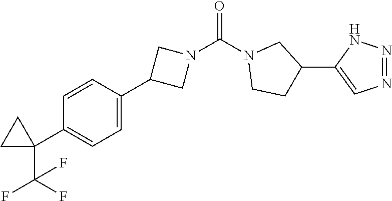 [SMILES: O=C(N1CC(c2ccc(C3(C(F)(F)F)CC3)cc2)C1)N1CCC(c2cnn[nH]2)C1] | Example 107 HPLC Method AD first eluting | 406.3 [M + H]⁺ |
| 113 | (−)- or (+)-[3- (1H-Triazol-5- yl)pyrro- lidin-1-yl]- [3-[4-[1-(tri- fluorometh- yl)cyclo- propyl]phe- nyl]azetidin-1- yl]methanone | 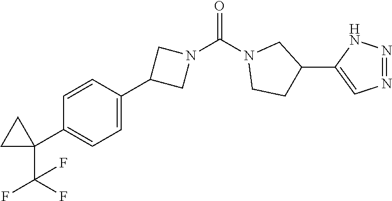 [SMILES: O=C(N1CC(c2ccc(C3(C(F)(F)F)CC3)cc2)C1)N1CCC(c2cnn[nH]2)C1] | Example 107 HPLC Method AD second eluting | 406.3 [M + H]⁺ |
| 114 | (+)- or (−)-[3- (3,3- Dimethyl-2H- benzofuran-6- yl)azetidin-1- yl]-[3-(1H- pyrazol- 5-yl)pyrrolidin- 1-yl]methanone | 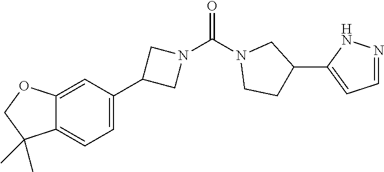 [SMILES: CC1(C)COc2cc(C3CN(C(=O)N4CCC(c5ccn[nH]5)C4)C3)ccc21] | BB23 SFC Method OD first eluting | 367.2 [M + H]⁺ |
| 115 | (−)- or (+)-[3- (3,3-Dimeth- yl-2H-benzo- furan-6-yl) azetidin-1-yl]- [3-(1H-pyrazol- 5-yl)pyrrolidin- 1-yl]meth- anone | 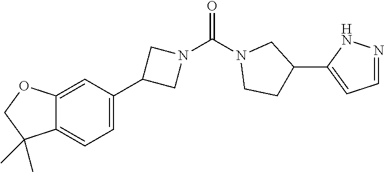 [SMILES: CC1(C)COc2cc(C3CN(C(=O)N4CCC(c5ccn[nH]5)C4)C3)ccc21] | BB23 SFC Method OD second eluting | 367.2 [M + H]⁺ |
| 116 | (+)- or (−)-[4- [bis(4-Fluoro- phenyl)meth- yl]-1-piperi- dyl]-[3-(1H- triazol-5-yl) pyrrolidin-1- yl]methanone | 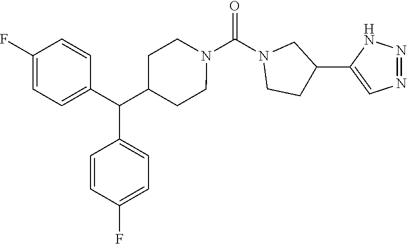 [SMILES: O=C(N1CCC(C(c2ccc(F)cc2)c2ccc(F)cc2)CC1)N1CCC(c2cnn[nH]2)C1] | Example 108 HPLC Method AD first eluting | [M + H]⁺ |
| 117 | (−)- or (+)-[4- [bis(4-Fluoro- phenyl)meth- yl]-1-piperi- dyl]-[3-(1H- triazol-5-yl) pyrrolidin-1- yl]methanone | 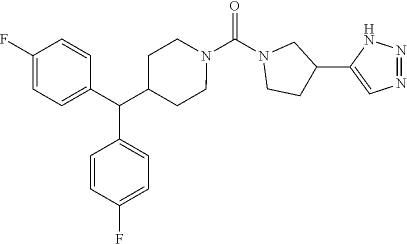 [SMILES: O=C(N1CCC(C(c2ccc(F)cc2)c2ccc(F)cc2)CC1)N1CCC(c2cnn[nH]2)C1] | Example 108 HPLC Method AD second eluting | 452.4 [M + H]⁺ |
| 156 | (+)- or (−)-[3- (4H-1,2,4- Triazol-3-yl) pyrrolidin- 1-yl]-[3- [4-[3-(2,2,2- trifluoroeth- oxy)aze- tidin-1-yl] phenyl]aze- tidin-1- yl]methanone | 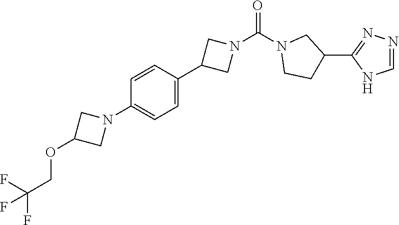 [SMILES: O=C(N1CC(c2ccc(N3CC(OCC(F)(F)F)C3)cc2)C1)N1CCC(c2nnc[nH]2)C1] | Example 145 SFC Method CE first eluting | 451.2 [M + H]⁺ |
| 157 | (−)- or (+)-[3- (4H-1,2,4-Tria- zol-3-yl)pyrro- lidin-1-yl]-[3- [4-[3-(2,2,2- trifluoro- ethoxy)aze- tidin-1-yl] phenyl]aze- tidin-1- yl]methanone | 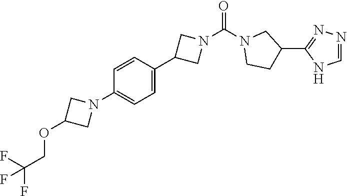 [SMILES: O=C(N1CC(c2ccc(N3CC(OCC(F)(F)F)C3)cc2)C1)N1CCC(c2nnc[nH]2)C1] | Example 145 SFC Method CE second eluting | 451.3 [M + H]⁺ |
| 158 | (+)- or (−)-[6- (2-Chloro- 4-fluorophe- noxy)-2-aza- spiro[3.3]hep- tan-2- yl]-[3-(4H- 1,2,4-triazol-3- yl)pyrrolidin-1- yl]methanone | 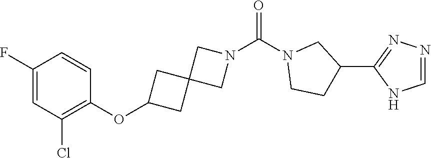 [SMILES: O=C(N1CCC(c2nnc[nH]2)C1)N1CC2(CC(Oc3ccc(F)cc3Cl)C2)C1] | Example 146 SFC Method CE first eluting | 406.2 [M + H]⁺ |
| 159 | (−)- or (+)-[6- (2-Chloro- 4-fluorophe- noxy)-2-aza- spiro[3.3]hep- tan-2-yl]- [3-(4H- 1,2,4-triazol-3- yl)pyrrolidin-1- yl]methanone | 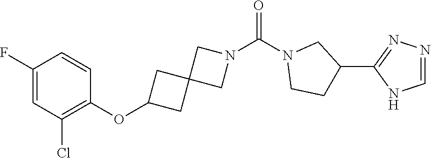 [SMILES: O=C(N1CCC(c2nnc[nH]2)C1)N1CC2(CC(Oc3ccc(F)cc3Cl)C2)C1] | Example 146 SFC Method CE second eluting | 406.2 [M + H]⁺ |
| 160 | (+)- or (−)-[3- (4H-1,2,4-Tri- azol-3-yl) pyrrolidin-1- yl]-[3-[6-[4- (trifluoro- methoxy)phe- noxy]-3-py- | 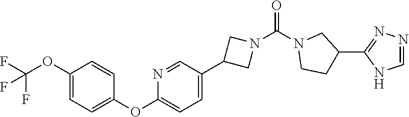 [SMILES: O=C(N1CC(c2ccc(Oc3ccc(OC(F)(F)F)cc3)nc2)C1)N1CCC(c2nnc[nH]2)C1] | Example 147 SFC Method CE first eluting | 475.2 [M + H]⁺ |
| | ridyl]azetidin- | | | |
| | 1-yl]methanone | | | |
| 161 | (−)- or (+)-[3- (4H-1,2,4- Triazol-3- yl)pyrrolidin-1- yl]-[3-[6-[4-(tri- fluoromethoxy) phenoxy]-3- pyridyl]azeti- | 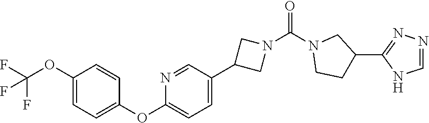 [SMILES: O=C(N1CC(c2ccc(Oc3ccc(OC(F)(F)F)cc3)nc2)C1)N1CCC(c2nnc[nH]2)C1] | Example 147 SFC Method CE second eluting | 475.2 [M + H]⁺ |
| | din-1-yl] | | | |
| | methanone | | | |
| 162 | (+)- or (−)-[3- [4-(4-Fluoro- phenoxy)phe- nyl]azetidin-1- yl]-[3-(tetrazol- 1-yl)pyrrolidin- 1-yl]methanone | 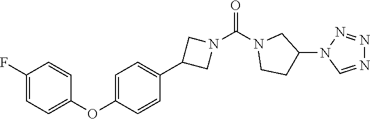 [SMILES: O=C(N1CC(c2ccc(Oc3ccc(F)cc3)cc2)C1)N1CCC(n2cnnn2)C1] | Example 153 SFC Method OJH2 first eluting | 409.2 [M + H]⁺ |
| 163 | (−)- or (+)-[3- [4-(4-Fluoro- phenoxy)phe- nyl]azetidin-1- yl]-[3-(tetra- zol-1- yl)pyrrolidin-1- yl]methanone | 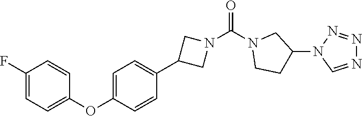 [SMILES: O=C(N1CC(c2ccc(Oc3ccc(F)cc3)cc2)C1)N1CCC(n2cnnn2)C1] | Example 153 SFC Method OJH2 second eluting | 409.2 [M + H]⁺ |
| 164 | (+)- or (−)-[3- (Tetrazol-1-yl) pyrrolidin-1- yl]-[6-[6- (trifluoro- methyl)pyra- zin-2-yl]oxy-2- azaspiro[3.3] | 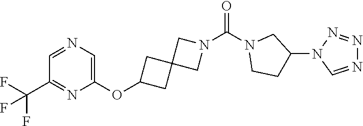 [SMILES: O=C(N1CCC(n2cnnn2)C1)N1CC2(CC(Oc3cncc(C(F)(F)F)n3)C2)C1] | Example 154 SFC Method OJH2 first eluting | 425.2 [M + H]⁺ |
| | heptan-2-yl] | | | |
| | methanone | | | |
| 165 | (−)- or (+)-[3- (Tetrazol-1- yl)pyrrolidin-1- yl-[6-[6-(tri- fluorometh- yl)pyrazin- 2-yl]oxy-2- azaspiro[3.3] heptan-2-yl] methanone | 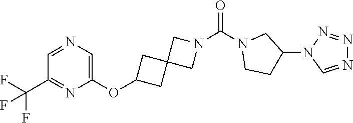 [SMILES: O=C(N1CCC(n2cnnn2)C1)N1CC2(CC(Oc3cncc(C(F)(F)F)n3)C2)C1] | Example 154 SFC Method OJH2 second eluting | 425.2 [M + H]⁺ |
| 166 | (+)- or (−)-[3- (Tetrazol-1-yl) pyrrolidin-1- yl]-[3-[6-[4- (trifluorometh- oxy)phenoxy]- 3-pyridyl]azeti- din-1-yl]metha- | 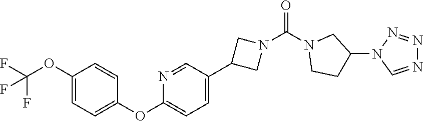 [SMILES: O=C(N1CC(c2ccc(Oc3ccc(OC(F)(F)F)cc3)nc2)C1)N1CCC(n2cnnn2)C1] | Example 155 SFC Method CE first eluting | 476.2 [M + H]⁺ |
| | none | | | |
| 167 | (−)- or (+)-[3- (Tetrazol-1-yl) pyrrolidin-1- yl]-[3-[6-[4- (trifluoro- methoxy)phe- noxy]-3-py- ridyl]azetidin- | 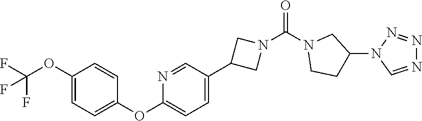 [SMILES: O=C(N1CC(c2ccc(Oc3ccc(OC(F)(F)F)cc3)nc2)C1)N1CCC(n2cnnn2)C1] | Example 155 SFC Method CE second eluting | 476.2 [M + H]⁺ |
| | 1-yl]metha- | | | |
| | none | | | |
| 228 | (3S)-1-[3-[4- (4-Chloro-2- methylsulfo- nylphenyl) phenyl]aze- tidine-1-car- bonyl]pyrroli- dine- 3-carboxamide | 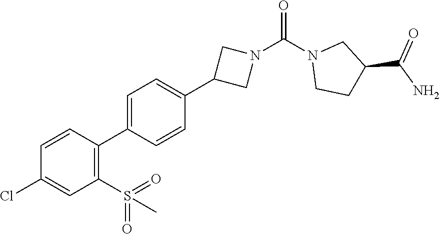 [SMILES: CS(=O)(=O)c1cc(Cl)ccc1-c1ccc(C2CN(C(=O)N3CC[C@H](C(N)=O)C3)C2)cc1] | Example 217 SFC Method IH first eluting | 462.2 [M + H]⁺ |
| 229 | (3R)-1-[3-[4- (4-Chloro-2- methylsulfonyl- phenyl)phenyl] azetidine-1-car- bonyl]pyrroli- dine- 3-carboxamide | 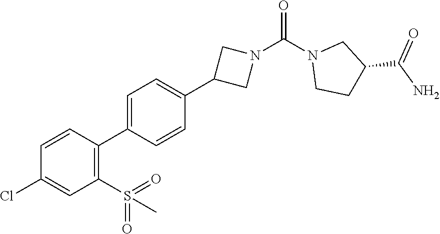 [SMILES: CS(=O)(=O)c1cc(Cl)ccc1-c1ccc(C2CN(C(=O)N3CC[C@@H](C(N)=O)C3)C2)cc1] | Example 217 SFC Method IH second eluting | 462.2 [M + H]⁺ |
| 268 | (3S)-1-[3-[4-[3- (2,2,2-Tri- fluoroethoxy) azetidin-1-yl] phenyl]azeti- dine-1- carbonyl] pyrrolidine- 3-carboxamide | 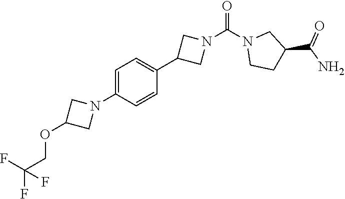 [SMILES: NC(=O)[C@H]1CCN(C(=O)N2CC(c3ccc(N4CC(OCC(F)(F)F)C4)cc3)C2)C1] | Example 254 SFC Method OJH1 30% MeOH first eluting | 427.3 [M + H]⁺ |
| 269 | (3R)-1-[3-[4- [3-(2,2,2-Tri- fluoroethoxy) azetidin-1-yl] phenyl]aze- tidine- 1-carbonyl] pyrrolidine- 3-carboxamide | 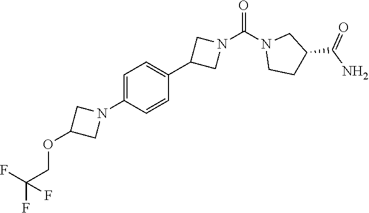 [SMILES: NC(=O)[C@@H]1CCN(C(=O)N2CC(c3ccc(N4CC(OCC(F)(F)F)C4)cc3)C2)C1] | Example 254 SFC Method OJH1 30% MeOH second eluting | 427.3 [M + H]⁺ |
| 305 | (3S)-1-[3-[4-(4- Chloro-2- methylsulfonyl- phenyl)phenyl] azetidine-1-car- bonyl]-N- methyl- pyrrolidine-3- carboxamide | 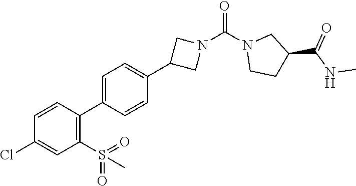 [SMILES: CNC(=O)[C@H]1CCN(C(=O)N2CC(c3ccc(-c4ccc(Cl)cc4S(C)(=O)=O)cc3)C2)C1] | Example 296 SFC Method ADH 50% MeOH first eluting | 476.2 [M + H]⁺ |
| 306 | (3R)-1-[3-[4- (4-Chloro-2- methylsulfonyl- phenyl)phenyl] azetidine-1-car- bonyl]-N-meth yl-pyrrolidine- 3-carbox- amide | 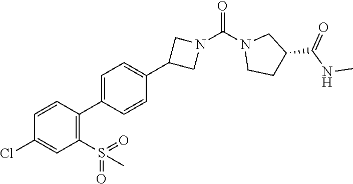 [SMILES: CNC(=O)[C@@H]1CCN(C(=O)N2CC(c3ccc(-c4ccc(Cl)cc4S(C)(=O)=O)cc3)C2)C1] | Example 296 SFC Method ADH 50% MeOH second eluting | 476.2 [M + H]⁺ |
| 329 | (−)- or (+)-[3- [4-(4-Chloro-2- methylsulfonyl- phenyl)phenyl] azetidin-1-yl]- [3-hydroxy- pyrrolidin- 1-yl] methanone | 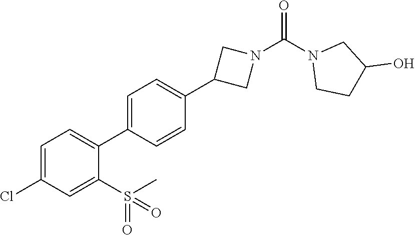 [SMILES: CS(=O)(=O)c1cc(Cl)ccc1-c1ccc(C2CN(C(=O)N3CCC(O)C3)C2)cc1] | [3-[4-(4- chloro-2- methylsulfo- nyl-phenyl) phenyl]aze- tidin-1-yl]- (3-hydroxy- pyrrolidin-1- yl)metha none (BB60) SFC Method ODH1 second eluting | 435.1 [M + H]⁺ |
| 392 | (−)- or (+)-[3- [4-(4-Chloro-2- methylsulfonyl- phenyl)phenyl] azetidin-1-yl]- [3-hydroxy- pyrrolidin- 1-yl]metha- none | 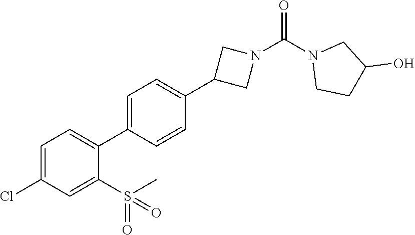 [SMILES: CS(=O)(=O)c1cc(Cl)ccc1-c1ccc(C2CN(C(=O)N3CCC(O)C3)C2)cc1] | [3-[4-(4- chloro-2- methylsul- fonylphenyl) phenyl]aze- tidin-1-yl]- (3-hydroxy- pyrrolidin-1- yl)metha- none (BB60) SFC Method ODH1 first eluting | 435.1 [M + H]⁺ |
| 330 | (−)- or (+)- [3-[4-(4- Chloro-2- methylsulfonyl- phenyl)phenyl] azetidin-1-yl]- [3-(hydroxy- methyl) pyrrolidin-1- yl]methanone | 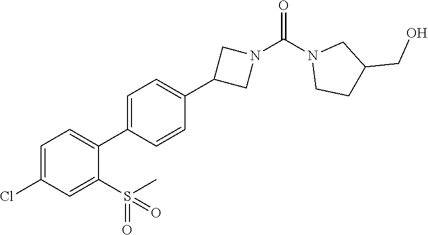 [SMILES: CS(=O)(=O)c1cc(Cl)ccc1-c1ccc(C2CN(C(=O)N3CCC(CO)C3)C2)cc1] | [3-[4-(4- Chloro- 2-methylsul- fonylphenyl) phenyl]aze- tidin-1-yl]- [3-(hydroxy- methyl)py- rrolidin-1- yl]metha- none (BB61) SFC Method IA 40% MeOH | 449.2 [M + H]⁺ |
| | | | first eluting | |
| 331 | (−)- or (+)-[3- [4-(4-Chloro-2- methylsulfonyl- phenyl)phe- nyl]azetidin- 1-yl]-[(3S)-3- (hydroxy- methyl) pyrrolidin-1- yl]methanone | 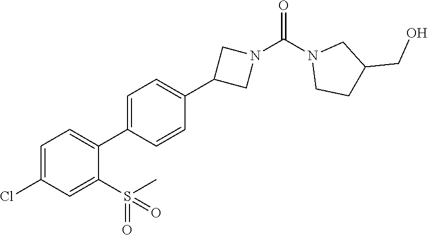 [SMILES: CS(=O)(=O)c1cc(Cl)ccc1-c1ccc(C2CN(C(=O)N3CCC(CO)C3)C2)cc1] | [3-[4-(4- Chloro- 2-methyl sulfonyl- phenyl) phenyl]aze- tidin-1-yl]- [3-(hy- droxy- methyl) pyrrolidin-1- yl]metha- none (BB61) SFC | 449.2 [M + H]⁺ |
| | | | Method IA | |
| | | | 40% MeOH | |
| | | | second | |
| | | | eluting | |
| 458 | (3R)-1-[3-[4-(4- Chloro-2- methylsulfonyl- phenyl)phenyl] azetidine-1- carbonyl]pyrro- lidine-3- sulfonamide | 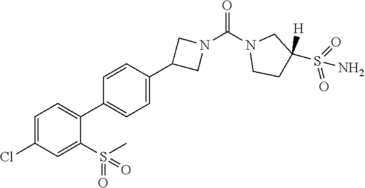 [SMILES: CS(=O)(=O)c1cc(Cl)ccc1-c1ccc(C2CN(C(=O)N3CC[C@@H](S(N)(=O)=O)C3)C2)cc1] | Example 359 SFC Method IH 25% MeOH First eluting | 498.1 [M + H]⁺ |
| 459 | (3S)-1-[3-[4-(4- Chloro-2- methylsulfonyl- phenyl)phenyl] azetidine-1-car- bonyl]pyrroli- dine-3- sulfonamide | 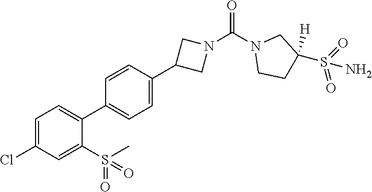 [SMILES: CS(=O)(=O)c1cc(Cl)ccc1-c1ccc(C2CN(C(=O)N3CC[C@H](S(N)(=O)=O)C3)C2)cc1] | Example 359 SFC Method IH 25% MeOH Second eluting | 498.1 [M + H]⁺ |
| 478 | (5S)-3-[3-[4-(4- Chloro-2- methylsulfonyl- phenyl)phenyl] azetidine-1- carbonyl]-4,5- dihydro- isoxazole-5- carboxamide | 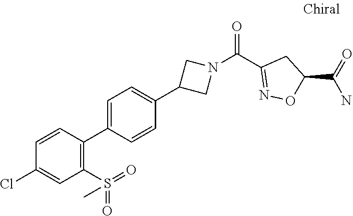 [SMILES: CS(=O)(=O)c1cc(Cl)ccc1-c1ccc(C2CN(C(=O)C3=NO[C@H](C(N)=O)C3)C2)cc1] | Example 460 SFC Method IH 40% MeOH First eluting | 462.1 [M + H]⁺ |

### Example 6

### [3-[[2-Fluoro-4-(trifluoromethyl)phenyl]methoxy]azetidin-1-yl]-[3-(4-methyl-1H-pyrazol-5-yl)azetidin-1-yl]methanone

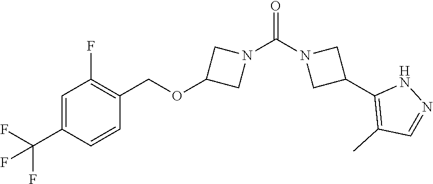 [SMILES: Cc1cn[nH]c1C1CN(C(=O)N2CC(OCc3ccc(C(F)(F)F)cc3F)C2)C1]

To a solution of 5-(azetidin-3-yl)-4-methyl-1H-pyrazole 2,2,2-trifluoroacetic acid (38 mg, 151 μmol, CAS RN 1361115-19-6) in DCM (1 mL) was added DIPEA (79 μL, 151 μmol) followed by addition of di(1H-1,2,4-triazol-1-yl)methanone (23.6 mg, 144 μmol) in one portion. The mixture was stirred at RT for 1 hour. After addition of 3-((2-Fluoro-4-(trifluoromethyl)benzyl)oxy)azetidine 4-methylbenzenesulfonic acid (CAS RN 2135785-94-1) in one portion, the clear solution was stirred at 50° C. overnight. The reaction mixture was completely evaporated. The product was purified by HPLC Method GN (except otherwise stated) to get the desired compound as a colorless amorphous (0.007 g; 11.2%). MS (ESI): m/z=413.2 [M+H]⁺.

If not indicated otherwise the following examples were synthesized in analogy to the synthesis described for Example 6 using suitable building blocks, respectively. The reactions were carried out in DCM, DMF or MeCN as a solvent between 50° C. and 80° C.

| | | | | MS |
|---|---|---|---|---|
| Ex. | Systematic Name | Structure | Building blocks | m/z |
| 7 | [3-[[2-Fluoro-4- (trifluoromethyl) phenyl]methoxy] azetidin-1-yl]- [3-(1H- pyrazol-5- yl)azetidin-1- yl]methanone | 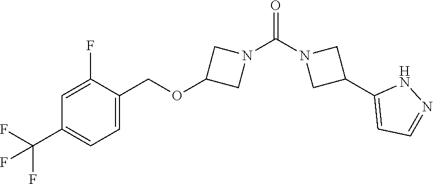 [SMILES: O=C(N1CC(OCc2ccc(C(F)(F)F)cc2F)C1)N1CC(c2ccn[nH]2)C1] | 3-((2-Fluoro-4- (trifluoromethyl) benzyl)oxy) azetidine 4- methylbenzene- sulfonic acid (CAS RN 2135785-94-1) and 5-(Azetidin- 3-yl)-1H-pyrazole 2,2,2-trifluoroacetic acid (CAS RN 2225154-19-6) | 399.1 [M + H]⁺ |
| 36 | [3-[[2-Fluoro-4- (trifluoromethyl) phenyl]methoxy] azetidin-1-yl]- [3-(1H-triazol-5- yl)pyrrolidin-1- yl]methanone | 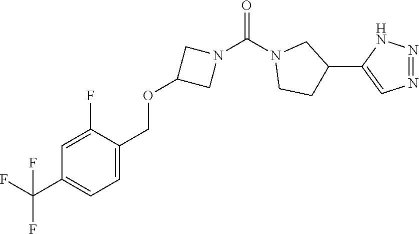 [SMILES: O=C(N1CC(OCc2ccc(C(F)(F)F)cc2F)C1)N1CCC(c2cnn[nH]2)C1] | 3-((2-Fluoro-4- (trifluoromethyl) benzyl)oxy) azetidine 4- methylbenzene- sulfonic acid (CAS RN 2135785-94-1) and 5-Pyrrolidin- 3-yl-1H-triazole (CAS RN 1517602- 91-3) | 414.2 [M + H]⁺ |
| 39 | [3-[[2-Fluoro-4- (trifluoromethyl) phenyl]methoxy] azetidin-1-yl]-[3- (1,2,4-triazol-1- yl)pyrrolidin-1- yl]methanone | 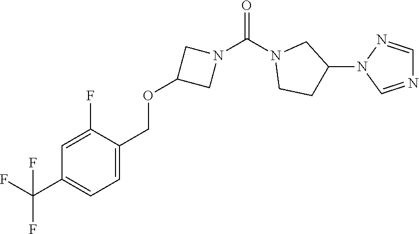 [SMILES: O=C(N1CC(OCc2ccc(C(F)(F)F)cc2F)C1)N1CCC(n2cncn2)C1] | 3-((2-Fluoro-4- (trifluoromethyl) benzyl)oxy) azetidine 4- methylbenzene- sulfonic acid (CAS RN 2135785-94-1) and 1-Pyrrolidin-3- yl-1,2,4-triazole (CAS RN 708204-49-3) | 414.2 [M + H]⁺ |
| 40 | [3-[[2-Fluoro-4- (trifluoromethyl) phenyl]methoxy] azetidin-1-yl]-(3- imidazol-1- ylpyrrolidin-1- yl)methanone | 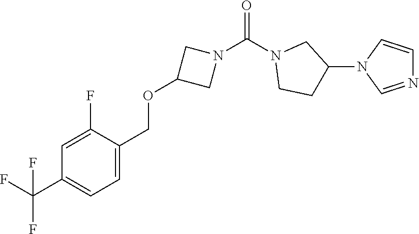 [SMILES: O=C(N1CC(OCc2ccc(C(F)(F)F)cc2F)C1)N1CCC(n2ccnc2)C1] | 3-((2-Fluoro-4- (trifluoromethyl) benzyl)oxy) azetidine 4- methylbenzene- sulfonic acid (CAS RN 2135785-94-1) and 1-Pyrrolidin-3- ylimidazole (CAS RN 64074-20-0) | 413.2 [M + H]⁺ |
| 41 | [3-[[2,4-bis (Trifluoromethyl) phenyl]methoxy] azetidin-1-yl]-[3- (1H-1,2,4-triazol-5- yl)pyrrolidin-1- yl]methanone | 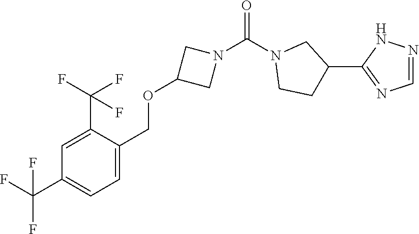 [SMILES: O=C(N1CC(OCc2ccc(C(F)(F)F)cc2C(F)(F)F)C1)N1CCC(c2ncn[nH]2)C1] | 3-[[2,4-bis (Trifluoromethyl) phenyl]methoxy] azetidine (CAS RN 1121598-55-7) and 3-Pyrrolidin-3-yl- 4H-1,2,4-triazole hydrochloride (CAS RN 1909337- 56-9) | 464.3 [M + H]⁺ |
| 50 | [3-[[2-Fluoro-4- (trifluoromethyl) phenyl]methoxy] azetidin-1-yl]-[3- (1H-imidazol-5- yl)pyrrolidin-1- yl]methanone | 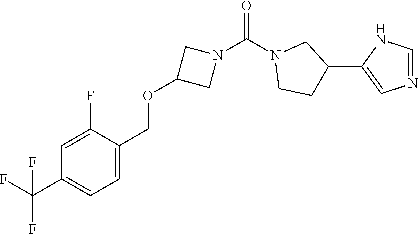 [SMILES: O=C(N1CC(OCc2ccc(C(F)(F)F)cc2F)C1)N1CCC(c2cnc[nH]2)C1] | 3-((2-Fluoro-4- (trifluoromethyl) benzyl)oxy) azetidine 4-methylbenzene- sulfonic acid (CAS RN 2135785-94-1) and 5-(Pyrrolidin-3- yl)-1H-imidazole (CAS RN 173590-38-0) | 413.2 [M + H]⁺ |
| 53 | [3-[[2-Fluoro-4- (trifluoromethyl) phenyl]methoxy] azetidin-1-yl]-[3- (1H-imidazol-2- yl)pyrrolidin-1- yl]methanone | 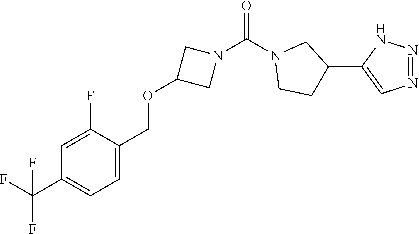 [SMILES: O=C(N1CC(OCc2ccc(C(F)(F)F)cc2F)C1)N1CCC(c2cnn[nH]2)C1] | 3-((2-Fluoro-4- (trifluoromethyl) benzyl)oxy) azetidine 4-methylbenzene- sulfonic acid (CAS RN 2135785-94-1) and 2-(Pyrrolidin-3- yl)-1H-imidazole dihydrochloride (CAS RN 1955555- 00-6) | 413.2 [M + H]⁺ |
| 58 | [3-[[3-Chloro-4- (trifluoromethyl) phenyl]methoxy] azetidin-1-yl]-[3- (1H-1,2,4-triazol- 5-yl)pyrrolidin-1- yl]methanone | 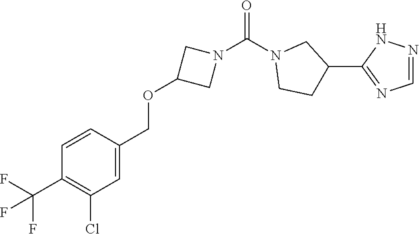 [SMILES: O=C(N1CC(OCc2ccc(C(F)(F)F)c(Cl)c2)C1)N1CCC(c2ncn[nH]2)C1] | 3-[[3-Chloro-4- (trifluoromethyl) phenyl]methoxy] azetidine 2,2,2- trifluoroacetic acid (CAS RN 2411574- 02-0) and 3- Pyrrolidin-3-yl-4H- 1,2,4-triazole hydrochloride (CAS RN 1909337- 56-9) | 430.2 [M + H]⁺ |
| 59 | [3-[[2-Chloro-4- (trifluoromethyl) phenyl]methoxy] azetidin-1-yl]-[3- (1H-1,2,4-triazol- 5-yl)pyrrolidin-1- yl]methanone | 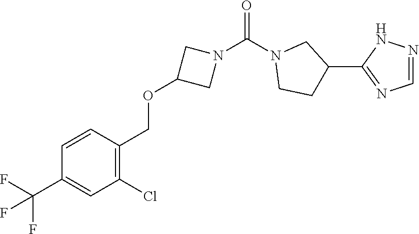 [SMILES: O=C(N1CC(OCc2ccc(C(F)(F)F)cc2Cl)C1)N1CCC(c2ncn[nH]2)C1] | 3-[[2-Chloro-4- (trifluoromethyl) phenyl]methoxy] azetidine 2,2,2- trifluoroacetic acid (CAS RN 2411573- 97-0) and 3- Pyrrolidin-3-yl-4H- 1,2,4-triazole hydrochloride (CAS RN 1909337- 56-9) | 430.2 [M + H]⁺ |
| 66 | [3-[[2-Fluoro-4- (trifluoromethyl) phenyl]methoxy] azetidin-1-yl]-[3- (1H-tetrazol-5- yl)pyrrolidin-1- yl]methanone | 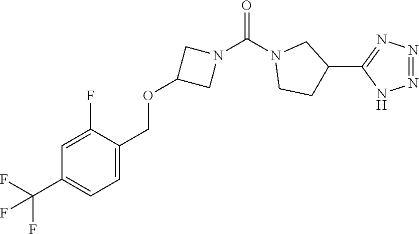 [SMILES: O=C(N1CC(OCc2ccc(C(F)(F)F)cc2F)C1)N1CCC(c2nnn[nH]2)C1] | 3-((2-Fluoro-4- (trifluoromethyl) benzyl)oxy) azetidine 4-methylbenzene- sulfonic acid (CAS RN 2135785-94-1) and 5-Pyrrolidin-3-yl- 1H-tetrazole (CAS RN 1228583-52-5) | 415.3 [M + H]⁺ |
| 78 | [3-[2-[2-fluoro-4- (trifluoromethyl) phenyl]ethyl] azetidin- 1-yl]-[(3S)-3-(1H- pyrazol-5- yl)pyrrolidin-1- yl]methanone | 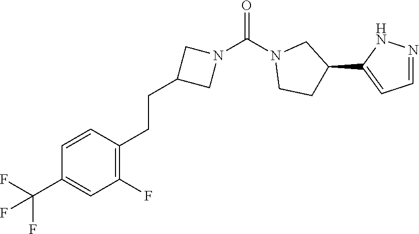 [SMILES: O=C(N1CC(CCc2ccc(C(F)(F)F)cc2F)C1)N1CC[C@H](c2ccn[nH]2)C1] | 3-[2-[2-Fluoro-4- (trifluoromethyl) phenyl]ethyl] azetidine 4- methylbenzene- sulfonic acid (CAS RN 2411577-40-5) and 5-[(3S)-Pyrrolidin- 3-yl]-1H-pyrazole (CAS RN 2165593- 33-7) | 411.3 [M + H]⁺ |
| 79 | [6-[(2,4- difluorophenyl) methyl]-2- azaspiro[3.3] heptan-2-yl]- [(3S)-3-(1H- pyrazol-5- yl)pyrrolidin-1- yl]methanone | 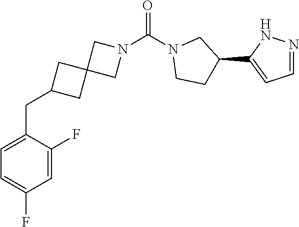 [SMILES: O=C(N1CC[C@H](c2ccn[nH]2)C1)N1CC2(CC(Cc3ccc(F)cc3F)C2)C1] | 6-[(2,4- Difluorophenyl) methyl]-2- azaspiro[3.3] heptane 2,2,2- trifluoroacetic acid (BB10) and 5- [(3S)-Pyrrolidin- 3-yl]-1H-pyrazole (CAS RN 2165593-33-7) | 387.3 [M + H]⁺ |
| 82 | [3-[[2-Fluoro-4- (trifluoromethyl) phenyl]methoxy] azetidin-1-yl]-(3- pyrrol-1- ylpyrrolidin-1- yl)methanone | 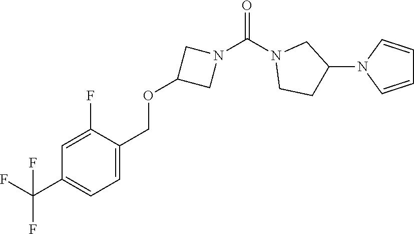 [SMILES: O=C(N1CC(OCc2ccc(C(F)(F)F)cc2F)C1)N1CCC(n2cccc2)C1] | 3-((2-Fluoro-4- (trifluoromethyl) benzyl)oxy) azetidine 4- methylbenzene- sulfonic acid (CAS RN 2135785-94-1) and 1-Pyrrolidin-3- ylpyrrole hydrochloride (CAS RN 1909326- 58-4) | 411.2 [M + H]⁺ |
| 88 | [3-[4-(2,4- Difluorophenyl) phenyl]azetidin- 1-yl]-[(3S)-3-(1H- pyrazol-5- yl)pyrrolidin-1- yl]methanone | 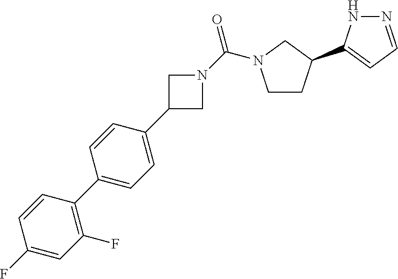 [SMILES: O=C(N1CC(c2ccc(-c3ccc(F)cc3F)cc2)C1)N1CC[C@H](c2ccn[nH]2)C1] | 3-[4-(2,4- Difluorophenyl) phenyl]azetidine 4-methylbenzene- sulfonic acid (BB12) and 5-[(3S)- Pyrrolidin-3-yl]- 1H-pyrazole (CAS RN 2165593-33-7) | 409.4 [M + H]⁺ |
| 107 | [3-(1H-Triazol-5- yl)pyrrolidin-1-yl]- [3-[4-[1- (trifluoromethyl) cyclopropyl] phenyl] azetidin-1- yl]methanone | 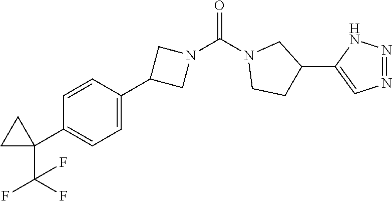 [SMILES: O=C(N1CC(c2ccc(C3(C(F)(F)F)CC3)cc2)C1)N1CCC(c2cnn[nH]2)C1] | 3-[4-[1- (Trifluoromethyl) cyclopropyl] phenyl] azetidine 4- methylbenzene- sulfonic acid (BB9) and 5-Pyrrolidin-3- yl-1H-triazole (CAS RN 1517602- 91-3) | 406.3 [M + H]⁺ |
| 108 | [4-[bis(4- Fluorophenyl) methyl]-1- piperidyl]-[3- (1H-triazol-5- yl)pyrrolidin-1- yl]methanone | 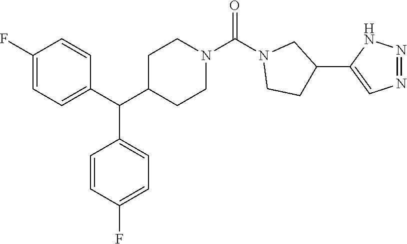 [SMILES: O=C(N1CCC(C(c2ccc(F)cc2)c2ccc(F)cc2)CC1)N1CCC(c2cnn[nH]2)C1] | 4-(bis(4- Fluorophenyl) methyl)piperidine (CAS RN 60285-00-9) and 5-Pyrrolidin- 3-yl-1H-triazole (CAS RN 1517602- 91-3) | 452.2 [M + H]⁺ |
| 119 | [3-[[2-Chloro-4- (trifluoromethyl) phenyl]methoxy] azetidin-1-yl]- [(3S)-3- (1H-triazol-5- yl)pyrrolidin-1- yl]methanone | 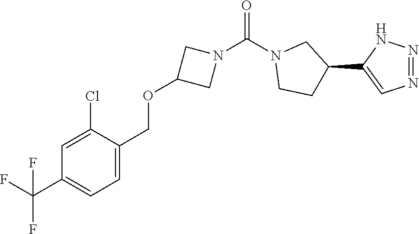 [SMILES: O=C(N1CC(OCc2ccc(C(F)(F)F)cc2Cl)C1)N1CC[C@H](c2cnn[nH]2)C1] | 3-[[2-Chloro-4- (trifluoromethyl) phenyl]methoxy] azetidine 2,2,2- trifluoroacetic acid (CAS RN 2411573- 97-0) and 5-[(3S)- Pyrrolidin-3-yl]- 1H-triazole (CAS RN 2166205-35-0) | 430.2 [M + H]⁺ |
| 120 | [(3S)-3-(1H- Triazol-5- yl)pyrrolidin- 1-yl]- [3-[4-(2,2,2- trifluoro-1,1- dimethyl- ethoxy)phenyl] azetidin-1-yl] methanone | 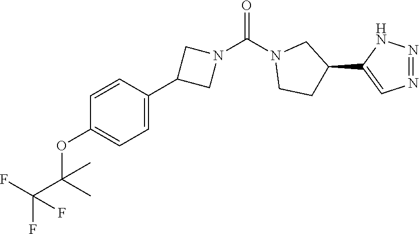 [SMILES: CC(C)(Oc1ccc(C2CN(C(=O)N3CC[C@H](c4cnn[nH]4)C3)C2)cc1)C(F)(F)F] | 3-(4-((1,1,1- Trifluoro-2- methylpropan-2- yl)oxy)phenyl) azetidine 4- methylbenzenesulf onic acid (BB24) and 5-[(3S)- Pyrrolidin-3-yl]- 1H-triazole (CAS RN 2166205-35-0) | 424.3 [M + H]⁺ |
| 121 | [3-[[2-Fluoro-4- (pentafluoro-26- sulfanyl)phenyl] methoxy] azetidin-1- yl]-[(3S)-3-(1H- triazol-5- yl)pyrrolidin-1- yl]methanone | 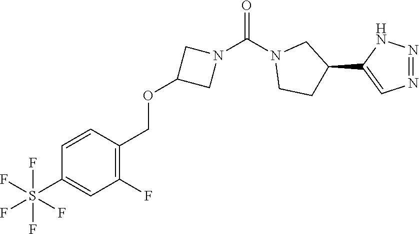 [SMILES: O=C(N1CC(OCc2ccc(S(F)(F)(F)(F)F)cc2F)C1)N1CC[C@H](c2cnn[nH]2)C1] | 3-((2-Fluoro-4- (pentafluoro-16- sulfaneyl)benzyl) oxy)azetidine 2,2,2- trifluoroacetic acid (BB2) and 5-[(3S)- Pyrrolidin-3-yl]- 1H-triazole (CAS RN 2166205-35-0) | 472.2 [M + H]⁺ |
| 122 | [6-[(2,4- Difluorophenyl) methyl]-2- azaspiro[3.3] heptan- 2-yl]-[(3S)-3- (1H-triazol-5- yl)pyrrolidin-1- yl]methanone | 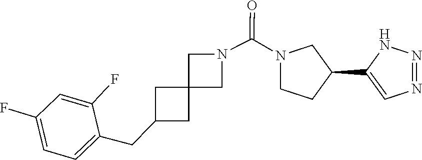 [SMILES: O=C(N1CC[C@H](c2cnn[nH]2)C1)N1CC2(CC(Cc3ccc(F)cc3F)C2)C1] | 6-[(2,4- Difluorophenyl) methyl]-2- azaspiro[3.3] heptane 2,2,2- trifluoroacetic acid (BB10) and 5- [(3S)-Pyrrolidin-3- yl]-1H-triazole (CAS RN 2166205- 35-0) | 388.2 [M + H]⁺ |
| 142 | [3-[6-(2- Chlorophenoxy)- 3-pyridyl] azetidin-1-yl]- [3-(tetrazol-1- yl)pyrrolidin-1- yl]methanone | 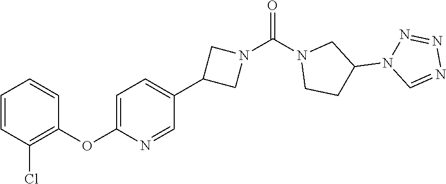 [SMILES: O=C(N1CC(c2ccc(Oc3ccccc3Cl)nc2)C1)N1CCC(n2cnnn2)C1] | 5-(Azetidin-3-yl)- 2-(2- chlorophenoxy) pyridine 4- methylbenzene- sulfonic acid (BB33) and 1-Pyrrolidin-3- yltetrazole (CAS RN 687622-87-3) | 426.2 [M + H]⁺ |
| 143 | [3-[[2-Fluoro-4- (trifluoromethyl) phenyl]methoxy] azetidin-1-yl]- [(3S)-3- (1,2,4-triazol-4- yl)pyrrolidin-1- yl]methanone | 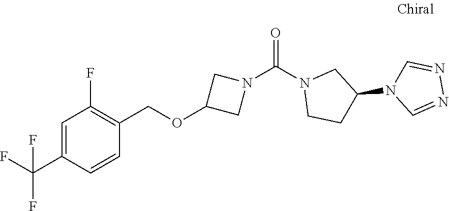 [SMILES: O=C(N1CC(OCc2ccc(C(F)(F)F)cc2F)C1)N1CC[C@H](n2cnnc2)C1] | 3-[[2-Fluoro-4- (trifluoromethyl) phenyl]methoxy] azetidine (CAS RN 1121597-80-5) and 4-[(3S)-Pyrrolidin- 3-yl]-1,2,4-triazole (CAS RN 1234191- 68-4) | 414.2 [M + H]⁺ |
| 144 | [3-[[2-Fluoro-4- (trifluoromethyl) phenyl]methoxy] azetidin-1-yl]- [(3R)-3- (1,2,4-triazol-4- yl)pyrrolidin-1- yl]methanone | 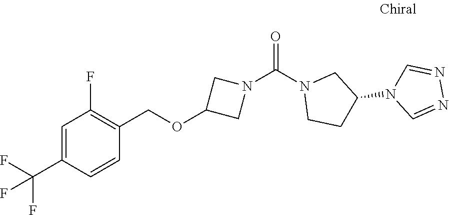 [SMILES: O=C(N1CC(OCc2ccc(C(F)(F)F)cc2F)C1)N1CC[C@@H](n2cnnc2)C1] | 3-[[2-Fluoro-4- (trifluoromethyl) phenyl]methoxy] azetidine (CAS RN 1121597-80-5) and 4-[(3R)-Pyrrolidin- 3-yl]-1,2,4-triazole (CAS RN 1234191- 65-1) | 414.1 [M + H]⁺ |
| 145 | [3-(4H-1,2,4- Triazol-3- yl)pyrrolidin-1- yl]-[3-[4- [3-(2,2,2- trifluoroethoxy) azetidin-1- yl]phenyl] azetidin- 1-yl]methanone | 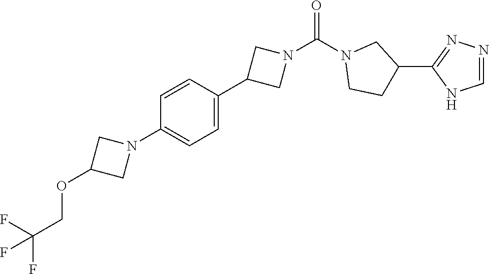 [SMILES: O=C(N1CC(c2ccc(N3CC(OCC(F)(F)F)C3)cc2)C1)N1CCC(c2nnc[nH]2)C1] | 1-[4-(Azetidin-3- yl)phenyl]-3-(2,2,2- trifluoroethoxy) azetidine 4- methylbenzene- sulfonic acid (BB34) and 3-Pyrrolidin-3- yl-4H-1,2,4-triazole (CAS RN 1339674- 44-0) | 451.2 [M + H]⁺ |
| 146 | [6-(2-Chloro-4- fluoro-phenoxy)- 2-azaspiro[3.3] heptan-2-yl]-[3- (4H-1,2,4- triazol-3- yl)pyrrolidin-1- yl]methanone | 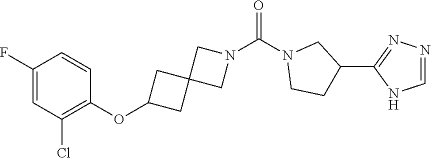 [SMILES: O=C(N1CCC(c2nnc[nH]2)C1)N1CC2(CC(Oc3ccc(F)cc3Cl)C2)C1] | 6-(2-Chloro-4- fluoro-phenoxy)- 2-azaspiro[3.3] heptane 2,2,2- trifluoroacetic acid (BB 1) and 3- Pyrrolidin-3-yl-4H- 1,2,4-triazole (CAS RN 1339674-44-0) | 406.1 [M + H]⁺ |
| 147 | [3-(4H-1,2,4- Triazol-3- yl)pyrrolidin-1- yl]-[3-[6-[4- (trifluoromethoxy) phenoxy]-3- pyridyl]azetidin- 1-yl]methanone | 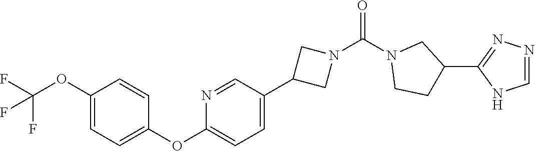 [SMILES: O=C(N1CC(c2ccc(Oc3ccc(OC(F)(F)F)cc3)nc2)C1)N1CCC(c2nnc[nH]2)C1] | 5-(Azetidin-3-yl)- 2-[4- (trifluoromethoxy) phenoxy]pyridine 4-methylbenzene- sulfonic acid (BB35) and 3-Pyrrolidin-3- yl-4H-1,2,4-triazole (CAS RN 1339674- 44-0) | 475.2 [M + H]⁺ |
| 148 | [(3S)-3-(1,2,4- Triazol-4- yl)pyrrolidin-1- yl]-[3-[5-[1- (trifluoromethyl) cyclopropyl]- 1,2,4-oxadiazol- 3-yl]azetidin-1- yl]methanone | 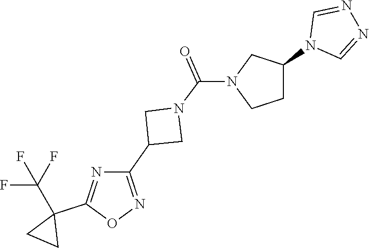 [SMILES: O=C(N1CC(c2noc(C3(C(F)(F)F)CC3)n2)C1)N1CC[C@H](n2cnnc2)C1] | 3-(Azetidin-3-yl)- 5-[1- (trifluoromethyl) cyclopropyl]-1,2,4- oxadiazole 4- methylbenzene- sulfonic acid (BB36) and 4-[(3S)- Pyrrolidin-3-yl]- 1,2,4-triazole (CAS RN 1234191-68-4) | 398.1 [M + H]⁺ |
| 149 | [(3S)-3-(1,2,4- Triazol-4- yl)pyrrolidin-1- yl]-[3-[4-[3- (2,2,2- trifluoroethoxy) azetidin-1- yl]phenyl] azetidin-1- yl]methanone | 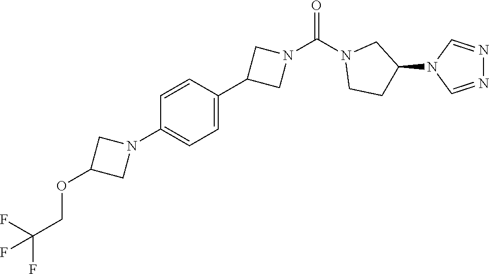 [SMILES: O=C(N1CC(c2ccc(N3CC(OCC(F)(F)F)C3)cc2)C1)N1CC[C@H](n2cnnc2)C1] | 1-[4-(Azetidin-3- yl)phenyl]-3-(2,2,2- trifluoroethoxy) azetidine 4- methylbenzene- sulfonic acid (BB34) and 4-[(3S)- Pyrrolidin-3-yl]- 1,2,4-triazole (CAS RN 1234191-68-4) | 451.2 [M + H]⁺ |
| 150 | [6-(2-Chloro-4- fluoro-phenoxy)- 2-azaspiro[3.3] heptan-2-yl]- [(3S)-3- (1,2,4-triazol-4- yl)pyrrolidin-1- yl]methanone | 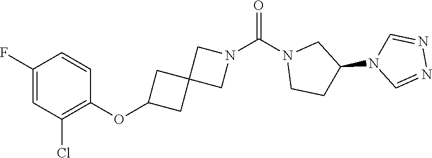 [SMILES: O=C(N1CC[C@H](n2cnnc2)C1)N1CC2(CC(Oc3ccc(F)cc3Cl)C2)C1] | 6-(2-Chloro-4- fluoro-phenoxy)-2- azaspiro[3.3] heptane 2,2,2- trifluoroacetic acid (BB 1) and 4-[(3S)- Pyrrolidin-3-yl]- 1,2,4-triazole (CAS RN 1234191-68-4) | 406.1 [M + H]⁺ |
| 151 | [(3R)-3-(1,2,4- Triazol-4- yl)pyrrolidin-1- yl]-[6-[6- (trifluoromethyl) pyrazin-2-yl]oxy- 2-azaspiro[3.3] heptan-2- yl]methanone | 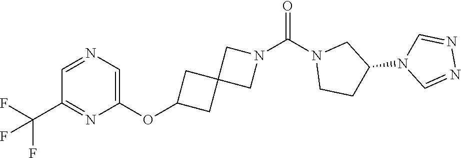 [SMILES: O=C(N1CC[C@@H](n2cnnc2)C1)N1CC2(CC(Oc3cncc(C(F)(F)F)n3)C2)C1] | 4-Methylbenzene- sulfonic acid 6-[6- (trifluoromethyl) pyrazin-2-yl]oxy-2- azaspiro[3.3]heptane (BB37) and 4- [(3R)-Pyrrolidin-3- yl]-1,2,4-triazole (CAS RN 1234191- 65-1) | 424.2 [M + H]⁺ |
| 152 | [(3R)-3-(1,2,4- Triazol-4- yl)pyrrolidin-1- yl]-[3-[6-[4- (trifluoromethoxy) phenoxy]-3- pyridyl]azetidin- 1-yl]methanone | 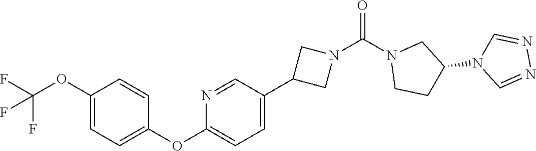 [SMILES: O=C(N1CC(c2ccc(Oc3ccc(OC(F)(F)F)cc3)nc2)C1)N1CC[C@@H](n2cnnc2)C1] | 5-(Azetidin-3-yl)- 2-[4- (trifluoromethoxy) phenoxy]pyridine 4- methylbenzenesulf onic acid (BB35) and 4-[(3R)- Pyrrolidin-3-yl]- 1,2,4-triazole (CAS RN 1234191-65-1) | 475.2 [M + H]⁺ |
| 153 | [3-[4-(4- Fluorophenoxy) phenyl]azetidin- 1-yl]-[3-(tetrazol- 1-yl)pyrrolidin-1- yl]methanone | 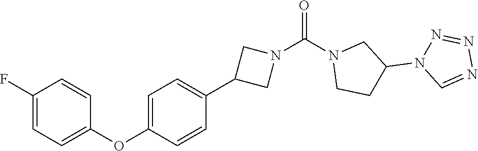 [SMILES: O=C(N1CC(c2ccc(Oc3ccc(F)cc3)cc2)C1)N1CCC(n2cnnn2)C1] | 3-[4-(4- Fluorophenoxy) phenyl]azetidine and (CAS RN 2358505- 83-4) and 1- Pyrrolidin-3- yltetrazole (CAS RN 687622-87-3) | 409.2 [M + H]⁺ |
| 154 | [3-(Tetrazol-1- yl)pyrrolidin-1- yl]-[6-[6- (trifluoromethyl) pyrazin-2-yl]oxy- 2-azaspiro[3.3] heptan-2- yl]methanone | 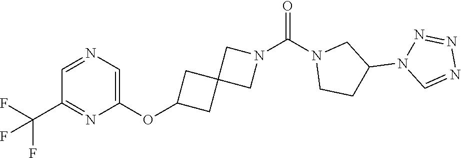 [SMILES: O=C(N1CCC(n2cnnn2)C1)N1CC2(CC(Oc3cncc(C(F)(F)F)n3)C2)C1] | 4- Methylbenzene- sulfonic acid 6-[6- (trifluoromethyl) pyrazin-2-yl]oxy-2- azaspiro[3.3]heptane (BB37) and 1- Pyrrolidin-3- yltetrazole (CAS RN 687622-87-3) | 425.2 [M + H]⁺ |
| 155 | [3-(Tetrazol-1- yl)pyrrolidin-1- yl]-[3-[6-[4- (trifluoromethoxy) phenoxy]-3- pyridyl]azetidin- 1-yl]methanone | 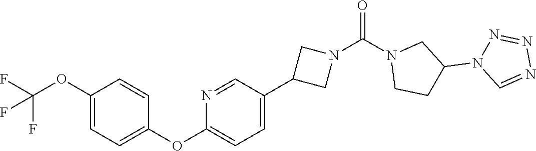 [SMILES: O=C(N1CC(c2ccc(Oc3ccc(OC(F)(F)F)cc3)nc2)C1)N1CCC(n2cnnn2)C1] | 5-(Azetidin-3-yl)- 2-[4- (trifluoromethoxy) phenoxy]pyridine 4- methylbenzenesulf onic acid (BB35) and 1-Pyrrolidin-3- yltetrazole (CAS RN 687622-87-3) | 476.2 [M + H]⁺ |
| 169 | [(3R)-3-(1H- Triazol-5- yl)pyrrolidin-1- yl]-[3-[5-[1- (trifluoromethyl) cyclopropyl]- 1,2,4-oxadiazol- 3-yl]azetidin-1- yl]methanone | 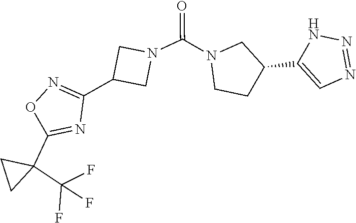 [SMILES: O=C(N1CC(c2noc(C3(C(F)(F)F)CC3)n2)C1)N1CC[C@@H](c2cnn[nH]2)C1] | 3-(Azetidin-3-yl)- 5-[1- (trifluoromethyl) cyclopropyl]-1,2,4- oxadiazole 4- methylbenzene- sulfonic acid (BB36) and 5-[(3R)- Pyrrolidin-3-yl]- 1H-triazole (CAS RN 2166044-57-9) | 398.1 [M + H]⁺ |
| 170 | [3-[4-(4- Fluorophenoxy) phenyl]azetidin- 1-yl]-[(3R)-3- (1H-triazol-5- yl)pyrrolidin-1- yl]methanone | 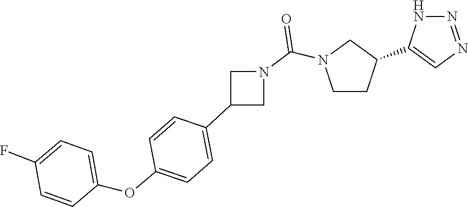 [SMILES: O=C(N1CC(c2ccc(Oc3ccc(F)cc3)cc2)C1)N1CC[C@@H](c2cnn[nH]2)C1] | 3-[4-(4- Fluorophenoxy) phenyl]azetidine and(CAS RN 2358505- 83-4) and 5-[(3R)- Pyrrolidin-3-yl]- 1H-triazole (CAS RN 2166044-57-9) | 408.2 [M + H]⁺ |
| 171 | [(3R)-3-(1H- Triazol-5- yl)pyrrolidin-1- yl]-[3-[4- [3-(2,2,2- trifluoroethoxy) azetidin-1- yl]phenyl] azetidin-1- yl]methanone | 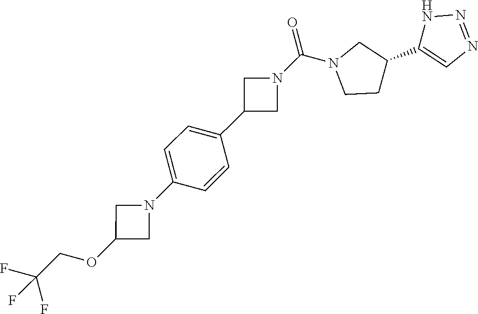 [SMILES: O=C(N1CC(c2ccc(N3CC(OCC(F)(F)F)C3)cc2)C1)N1CC[C@@H](c2cnn[nH]2)C1] | 1-[4-(Azetidin-3- yl)phenyl]-3-(2,2,2- trifluoroethoxy) azetidine 4- methylbenzene- sulfonic acid (BB34) and 5-[(3R)- Pyrrolidin-3-yl]- 1H-triazole (CAS RN 2166044-57-9) | 451.2 [M + H]⁺ |
| 172 | [6-(2-Chloro-4- fluoro-phenoxy)- 2-azaspiro[3.3] heptan-2-yl]- [(3R)-3-(1H- triazol-5- yl)pyrrolidin-1- yl]methanone | 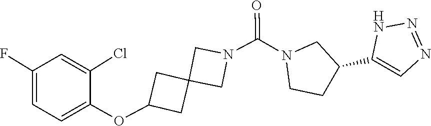 [SMILES: O=C(N1CC[C@@H](c2cnn[nH]2)C1)N1CC2(CC(Oc3ccc(F)cc3Cl)C2)C1] | 6-(2-Chloro-4- fluoro-phenoxy)- 2-azaspiro[3.3] heptane 2,2,2- trifluoroacetic acid (BB 1) and 5-[(3R)- Pyrrolidin-3-yl]- 1H-triazole (CAS RN 2166044-57-9) | 406.1 [M + H]⁺ |
| 173 | [(3R)-3-(1H- Triazol-5- yl)pyrrolidin-1- yl]-[3-[6-[4- (trifluoromethoxy) phenoxy]-3- pyridyl]azetidin- 1-yl]methanone | 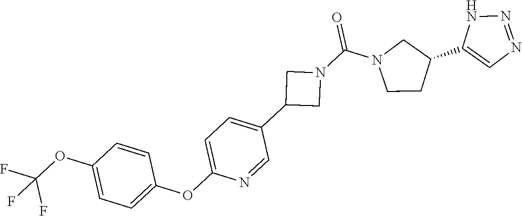 [SMILES: O=C(N1CC(c2ccc(Oc3ccc(OC(F)(F)F)cc3)nc2)C1)N1CC[C@@H](c2cnn[nH]2)C1] | 5-(Azetidin-3-yl)- 2-[4- (trifluoromethoxy) phenoxy]pyridine 4-methylbenzene- sulfonic acid (BB35) and 5-[(3R)- Pyrrolidin-3-yl]- 1H-triazole (CAS RN 2166044-57-9) | 475.1 [M + H]⁺ |
| 174 | [(3R)-3-(1H- Triazol-5- yl)pyrrolidin-1- yl]-[3-[6-[3- (trifluoromethyl) pyrrolidin-1-yl]-3- pyridyl]azetidin-1- yl]methanone | 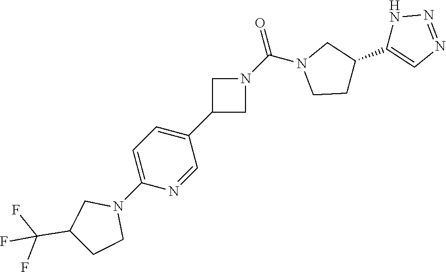 [SMILES: O=C(N1CC(c2ccc(N3CCC(C(F)(F)F)C3)nc2)C1)N1CC[C@@H](c2cnn[nH]2)C1] | 5-(Azetidin-3-yl)- 2-[3- (trifluoromethyl) pyrrolidin-1- yl]pyridine (BB39) and 5-[(3R)- Pyrrolidin-3-yl]- 1H-triazole (CAS RN 2166044-57-9) | 436.2 [M + H]⁺ |
| 175 | [3-[2-(3- Chlorophenyl) ethynyl]azetidin- 1-yl]- [(3R)-3-(1H- triazol-5- yl)pyrrolidin-1- yl]methanone | 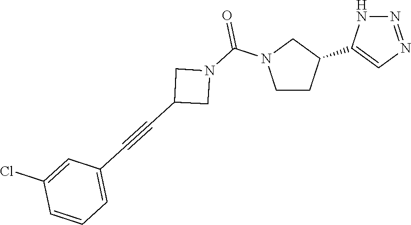 [SMILES: O=C(N1CC(C#Cc2cccc(Cl)c2)C1)N1CC[C@@H](c2cnn[nH]2)C1] | 3-[2-(3- Chlorophenyl) ethynyl]azetidine (CAS RN 2411575-81-8) and 5-[(3R)- Pyrrolidin-3-yl]- 1H-triazole (CAS RN 2166044-57-9) | 356.1 [M + H]⁺ |
| 176 | [3-[2-[2- (Difluoromethyl) phenyl]ethynyl] azetidin-1-yl]- [(3R)-3- (1H-triazol-5- yl)pyrrolidin-1- yl]methanone | 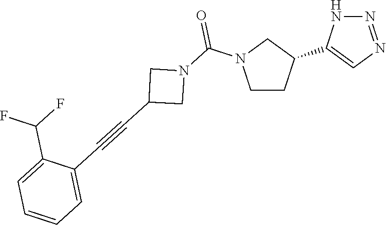 [SMILES: O=C(N1CC(C#Cc2ccccc2C(F)F)C1)N1CC[C@@H](c2cnn[nH]2)C1] | 3-[2-[2- (Difluoromethyl) phenyl]ethynyl] azetidine (CAS RN 2411575-99-8) and 5-[(3R)-Pyrrolidin- 3-yl]-1H-triazole (CAS RN 2166044- 57-9) | 372.1 [M + H]⁺ |
| 183 | [3-(4-tert- Butylphenyl) azetidin-1-yl]- [3-(1H- pyrazol-5- yl)pyrrolidin-1- yl]methanone | 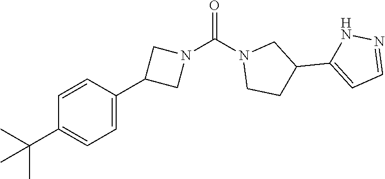 [SMILES: CC(C)(C)c1ccc(C2CN(C(=O)N3CCC(c4ccn[nH]4)C3)C2)cc1] | 3-(4-(tert- Butyl)phenyl) azetidine 4- methylbenzene- sulfonate (CAS RN 2377009-52-2) and 5-(pyrrolidin-3-yl)- 1H-pyrazole dihydrochloride (CAS RN 2173135-18-5) | 353.3 [M + H]⁺ |
| 184 | [3-[[2-Fluoro-4- (trifluoromethyl) phenyl]methoxy] azetidin-1-yl]-[3- (triazol-1- yl)pyrrolidin-1- yl]methanone | 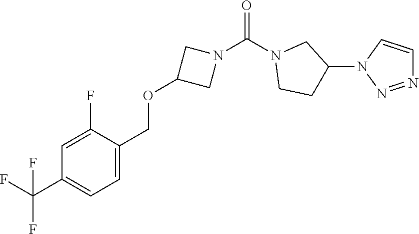 [SMILES: O=C(N1CC(OCc2ccc(C(F)(F)F)cc2F)C1)N1CCC(n2ccnn2)C1] | 3-((2-Fluoro-4- (trifluoromethyl) benzyl)oxy) azetidine 4- methylbenzene- sulfonate (CAS RN 2135785-94-1) and 1-(Pyrrolidin-3-yl)- 1H-1,2,3-triazole (CAS RN 169750- 38-3) | 414.2 [M + H]⁺ |
| 85 | [4-(3- Cyclopropyl- 1,2,4-oxadiazol- 5-yl)piperazin- 1-yl]- [rac-(3S)-3- (1H-triazol-5- yl)pyrrolidin-1- yl]methanone | 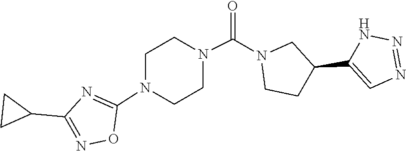 [SMILES: O=C(N1CCN(c2nc(C3CC3)no2)CC1)N1CC[C@H](c2cnn[nH]2)C1] | 4-(3-Cyclopropyl- 1,2,4-oxadiazol-5- yl)piperazin-1-ium chloride (CAS RN 2448340-71-2) and 5-[(3S)-Pyrrolidin- 3-yl]-1H-triazole (CAS RN 2166205- 35-0) | 359.1 [M + H]⁺ |
| 189 | [3-[4-(4-Chloro-2- methylsulfonyl- phenyl)phenyl] azetidin-1-yl]-[3- (triazol-1- yl)pyrrolidin-1- yl]methanone | 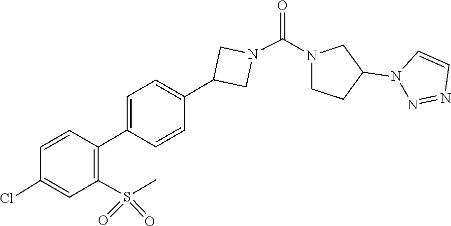 [SMILES: CS(=O)(=O)c1cc(Cl)ccc1-c1ccc(C2CN(C(=O)N3CCC(n4ccnn4)C3)C2)cc1] | 3-[4-(4-Chloro-2- methylsulfonyl- phenyl)phenyl] azetidine;4- methylbenzene- sulfonic acid (BB44) and 1-(Pyrrolidin- 3-yl)-1H-1,2,3- triazole (CAS RN 169750-38-3) | 486.2 [M + H]⁺ |
| 190 | [3-[4-[3-(2,2- Dimethylpropyl) triazol-4- yl]phenyl]azetidin- 1-yl]-[3-(triazol-1- yl)pyrrolidin-1- yl]methanone | 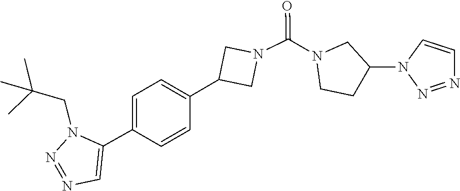 | 5-[4-(Azetidin-3- yl)phenyl]-1-(2,2- dimethylpropyl) triazole 4- methylbenzene- sulfonic acid (BB45) and 1-(Pyrrolidin- 3-yl)-1H-1,2,3- triazole (CAS RN 169750-38-3) | 479.4 [M + H]⁺ |
| 191 | [3-(Triazol-1- yl)pyrrolidin-1- yl]-[3-[4-[1- (trifluoromethyl) cyclopropyl] phenyl]azetidin- 1-yl]methanone | 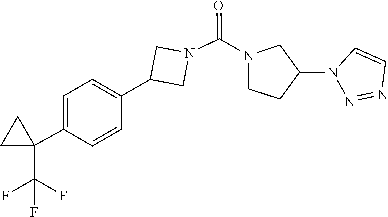 [SMILES: O=C(N1CC(c2ccc(C3(C(F)(F)F)CC3)cc2)C1)N1CCC(n2ccnn2)C1] | 3-[4-[1- (Trifluoromethyl) cyclopropyl]phenyl] azetidine 4- methylbenzene- sulfonic acid (BB9) and 1-(Pyrrolidin- 3-yl)-1H-1,2,3- triazole (CAS RN 169750-38-3) | 406.2 [M + H]⁺ |
| 192 | [3-(2-Chloro-4- isopropylsulfonyl- phenyl)azetidin- 1-yl]-[(3S)-3- (1H-triazol-5- yl)pyrrolidin-1- yl]methanone | 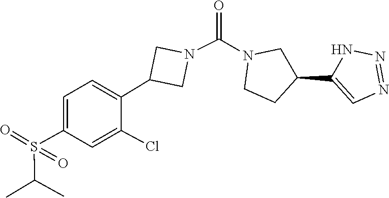 [SMILES: CC(C)S(=O)(=O)c1ccc(C2CN(C(=O)N3CC[C@H](c4cnn[nH]4)C3)C2)c(Cl)c1] | 3-(2-Chloro-4- isopropylsulfonyl- phenyl)azetidine; 4- methylbenzenesulf onic acid (BB46) and 5-[(3S)- Pyrrolidin-3-yl]- 1H-triazole (CAS RN 2166205-35-0) HPLC YMC | 438.2 [M + H]⁺ |
| 193 | [3-(4-tert- Butylsulfonyl-2- chloro- phenyl)azetidin- 1-yl]-[(3S)- 3-(1H- triazol-5- yl)pyrrolidin-1- yl]methanone | 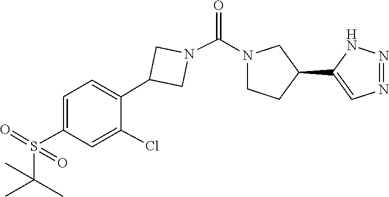 [SMILES: CC(C)(C)S(=O)(=O)c1ccc(C2CN(C(=O)N3CC[C@H](c4cnn[nH]4)C3)C2)c(Cl)c1] | 3-(4-tert- Butylsulfonyl-2- chloro- phenyl)azetidine;4- methylbenzene- sulfonic acid (BB47) and 5-[(3S)- Pyrrolidin-3-yl]- 1H-triazole (CAS RN 2166205-35-0) HPLC YMC | 452.2 [M + H]⁺ |
| 195 | [(3S)-3-(1H- Triazol-5- yl)pyrrolidin- 1-yl]-[6-[6- (trifluoromethyl) pyrazin-2-yl]oxy- 2-azaspiro[3.3] heptan-2-yl] methanone | 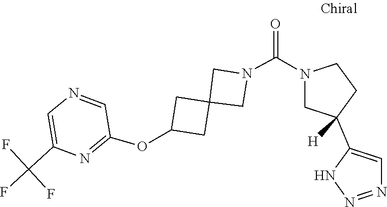 [SMILES: O=C(N1CC[C@H](c2cnn[nH]2)C1)N1CC2(CC(Oc3cncc(C(F)(F)F)n3)C2)C1] | 6-[6- (trifluoromethyl) pyrazin-2-yl]oxy-2- azaspiro[3.3] heptane; 4- methylbenzene- sulfonic acid (BB37) and 5-[(3S)- Pyrrolidin-3-yl]- 1H-triazole (CAS RN 2166205-35-0) | 424.2 [M + H]⁺ |
| 196 | [3-[4-(4- Chloro-2- methylsulfonyl- phenyl)phenyl] azetidin-1-yl]- [(3S)-3- (1H-triazol-5- yl)pyrrolidin-1- yl]methanone | 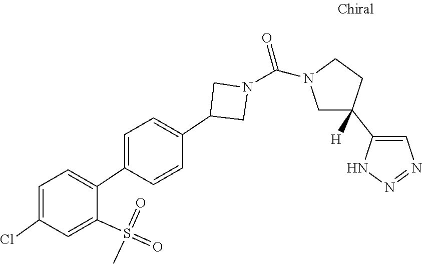 [SMILES: CS(=O)(=O)c1cc(Cl)ccc1-c1ccc(C2CN(C(=O)N3CC[C@H](c4cnn[nH]4)C3)C2)cc1] | 3-[4-(4-Chloro-2- methylsulfonyl- phenyl)phenyl] azetidine; 4- methylbenzene- sulfonic acid (BB44) and 5-[(3S)- Pyrrolidin-3-yl]- 1H-triazole (CAS RN 2166205-35-0) | 486.2 [M + H]⁺ |
| 199 | [3-[4-(4- Fluorophenoxy) phenyl]azetidin- 1-yl]-[(3S)-3- (1H-triazol- 5-yl)pyrrolidin- 1-yl]methanone | 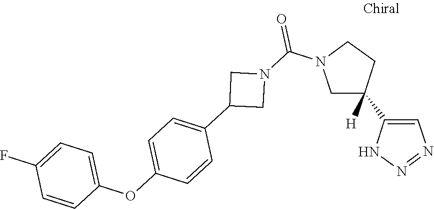 [SMILES: O=C(N1CC(c2ccc(Oc3ccc(F)cc3)cc2)C1)N1CC[C@H](c2cnn[nH]2)C1] | 3-[4-(4- Fluorophenoxy) phenyl]azetidine and (CAS RN 2358505- 83-4) and 5-[(3S)- Pyrrolidin-3-yl]- 1H-triazole (CAS RN 2166205-35-0) | 408.2 [M + H]⁺ |
| 200 | [3-(2-Chloro-4- isobutylsulfonyl- phenyl)azetidin- 1-yl]-[(3S)-3- (1H-triazol-5- yl)pyrrolidin-1- yl]methanone | 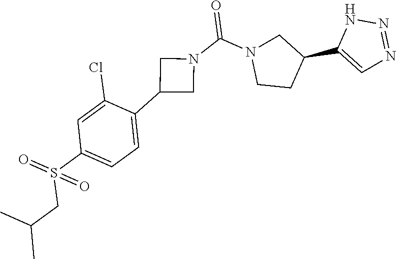 [SMILES: CC(C)CS(=O)(=O)c1ccc(C2CN(C(=O)N3CC[C@H](c4cnn[nH]4)C3)C2)c(Cl)c1] | 3-(2-Chloro-4- isobutylsulfonyl- phenyl)azetidine;4- methylbenzene- sulfonic acid (BB48) and 5-[(3S)- Pyrrolidin-3-yl]- 1H-triazole (CAS RN 2166205-35-0) HPLC YMC | 452.2 [M + H]⁺ |
| 203 | [3-[4- (Benzenesulfonyl) phenyl]azetidin-1- yl]-[(3S)-3-(1H- triazol-5- yl)pyrrolidin-1- yl]methanone | 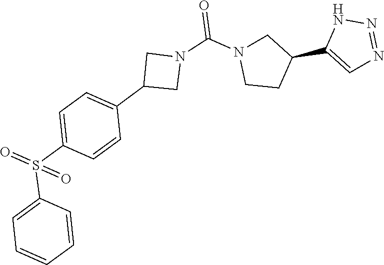 [SMILES: O=C(N1CC(c2ccc(S(=O)(=O)c3ccccc3)cc2)C1)N1CC[C@H](c2cnn[nH]2)C1] | 3-[4- (Benzenesulfonyl) phenyl]azetidine;4- methylbenzene- sulfonic acid (BB49) and 5-[(3S)- Pyrrolidin-3-yl]- 1H-triazole (CAS RN 2166205-35-0) HPLC YMC | 438.3 [M + H]⁺ |
| 203 | [3-[4-(2-Chloro- 4-methylsulfonyl- phenyl)phenyl] azetidin-1-yl]- [(3S)-3- (1H-triazol-5- yl)pyrrolidin-1- yl]methanone | 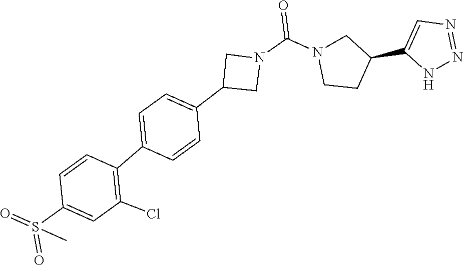 [SMILES: CS(=O)(=O)c1ccc(-c2ccc(C3CN(C(=O)N4CC[C@H](c5cnn[nH]5)C4)C3)cc2)c(Cl)c1] | 3-[4-(2-Chloro-4- methylsulfonyl- phenyl)phenyl] azetidine;4- methylbenzene- sulfonic acid (BB50) and 5-[(3S)- Pyrrolidin-3-yl]- 1H-triazole (CAS RN 2166205-35-0) HPLC YMC | 486.3 [M + H]⁺ |
| 204 | [3-(4-tert- Butylphenyl) azetidin-1-yl]- [(3S)-3- (1H-triazol-5- yl)pyrrolidin- 1-yl]methanone | 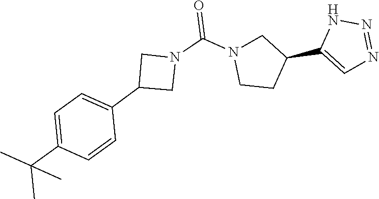 [SMILES: CC(C)(C)c1ccc(C2CN(C(=O)N3CC[C@H](c4cnn[nH]4)C3)C2)cc1] | 3-(4-tert- Butylphenyl) azetidine;4- methylbenzene- sulfonic acid (BB51) and 5-[(3S)- Pyrrolidin-3-yl]- 1H-triazole (CAS RN 2166205-35-0) HPLC YMC | 354.3 [M + H]⁺ |
| 210 | [3-[4-(N- Methylanilino) phenyl]azetidin- 1-yl]-[(3S)-3- (1H-triazol- 5-yl)pyrrolidin- 1-yl]methanone | 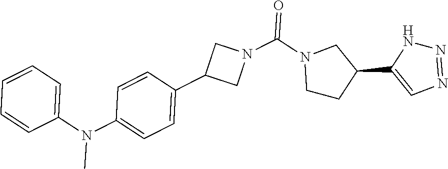 [SMILES: CN(c1ccccc1)c1ccc(C2CN(C(=O)N3CC[C@H](c4cnn[nH]4)C3)C2)cc1] | 4-(Azetidin-3-yl)- N-methyl-N- phenyl-aniline;4- methylbenzene- sulfonic acid (BB52) and 5-[(3S)- Pyrrolidin-3-yl]- 1H-triazole (CAS RN 2166205-35-0) HPLC YMC | |
| 211 | [(3S)-3-(1H- Triazol-5- yl)pyrrolidin- 1-yl]-[3-[4- (trifluoromethyl- sulfonyl)phenyl] azetidin-1- yl]methanone | 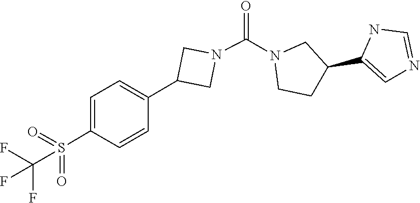 | 3-[4- (Trifluoromethyl- sulfonyl)phenyl] azetidine; 4- methylbenzene- sulfonic acid (BB53) and 5-[(3S)- Pyrrolidin-3-yl]- 1H-triazole (CAS RN 2166205-35-0) HPLC YMC | 430.2 [M + H]⁺ |
| 215 | [6-[[4-Fluoro-2- (trifluoromethyl) phenyl]methyl]- 2,6-diazaspiro [3.3]heptan-2- yl]-[(3S)-3- (1H-triazol-5- yl)pyrrolidin-1- yl]methanone | 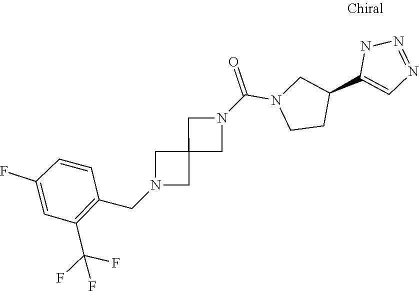 [SMILES: O=C(N1CC[C@H](c2cnn[nH]2)C1)N1CC2(CN(Cc3ccc(F)cc3C(F)(F)F)C2)C1] | 2-[[4-Fluoro-2- (trifluoromethyl) phenyl]methyl]-2,6- diazaspiro[3.3] heptane; trifluoroacetic acid (BB103) and 5-[(3S)-Pyrrolidin- 3-yl]-1H-triazole (CAS RN 2166205- 35-0) | 439.3 [M + H]⁺ |
| 216 | [3-[4-[N- (Cyclopropyl- methyl)anilino] phenyl]azetidin- 1-yl]-[(3S)- 3-(1H-triazol-5- yl)pyrrolidin-1- yl]methanone | 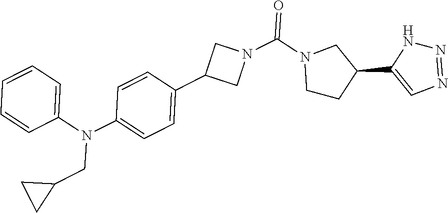 [SMILES: O=C(N1CC(c2ccc(N(CC3CC3)c3ccccc3)cc2)C1)N1CC[C@H](c2cnn[nH]2)C1] | 4-(Azetidin-3-yl)- N-(cyclopropyl- methyl)-N- phenyl-aniline (BB54) and 5- [(3S)-Pyrrolidin-3- yl]-1H-triazole (CAS RN 2166205- 35-0) HPLC YMC | 443.4 [M + H]⁺ |
| 217 | 1-[3-[4-(4-Chloro- 2-methylsulfonyl- phenyl)phenyl] azetidine-1- carbonyl] pyrrolidine-3- carboxamide | 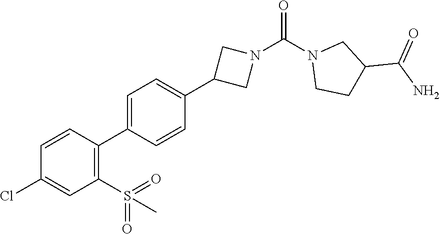 [SMILES: CS(=O)(=O)c1cc(Cl)ccc1-c1ccc(C2CN(C(=O)N3CCC(C(N)=O)C3)C2)cc1] | 3-[4-(4-Chloro-2- methylsulfonyl- phenyl)phenyl] azetidine;4- methylbenzene- sulfonic acid (BB44) and Pyrrolidine-3- carboxamide;hydro chloride ( CAS RN 644972-57-6) HPLC YMC | 462.2 [M + H]⁺ |
| 219 | [3-(6-tert-Butyl-3- pyridyl)azetidin- 1-yl]-[(3S)-3-(1H- triazol-5- yl)pyrrolidin-1- yl]methanone | 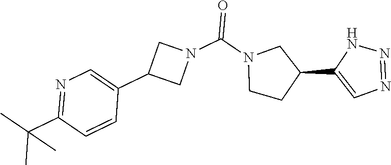 [SMILES: CC(C)(C)c1ccc(C2CN(C(=O)N3CC[C@H](c4cnn[nH]4)C3)C2)cn1] | 5-(Azetidin-3-yl)- 2-tert-butyl- pyridine (BB55) and 5-[(3S)- Pyrrolidin-3-yl]- 1H-triazole (CAS RN 2166205-35-0) | 355.3 [M + H]⁺ |
| 226 | [3-[6-[(3-Ethyl-2- pyridyl)-methyl- amino]-3- pyridyl]azetidin- 1-yl]-[(3S)-3- (1H-triazol-5- yl)pyrrolidin-1- yl]methanone | 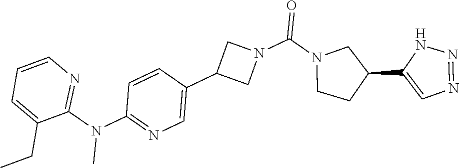 [SMILES: CCc1cccnc1N(C)c1ccc(C2CN(C(=O)N3CC[C@H](c4cnn[nH]4)C3)C2)cn1] | N-[5-(Azetidin-3- yl)-2-pyridyl]-3- ethyl-N-methyl- pyridin-2-amine (BB56) and 5- [(3S)-Pyrrolidin-3- yl]-1H-triazole (CAS RN 2166205- 35-0) | 433.3 [M + H]⁺ |
| 231 | [3-(1H-Tetrazol-5- yl)pyrrolidin-1- yl]-[3-[4-(2,2,2- trifluoro-1,1- dimethyl- ethoxy)phenyl] azetidin-1- yl]methanone | 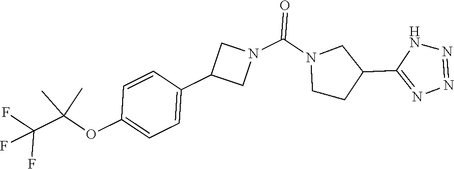 [SMILES: CC(C)(Oc1ccc(C2CN(C(=O)N3CCC(c4nnn[nH]4)C3)C2)cc1)C(F)(F)F] | 3-[4-(2,2,2- Trifluoro-1,1- dimethyl- ethoxy)phenyl] azetidine; 4- methylbenzene- sulfonic acid (BB57) and 5-pyrrolidin-3- yl-1H-tetrazole; hydrochloride (CAS RN | 425.3 [M + H]⁺ |
| | | | 1228583-52-5) | |
| | | | HPLC YMC | |
| 233 | [2-[4-Fluoro-2- (trifluoromethyl) phenyl]sulfonyl- 2,6-diazaspiro [3.3]heptan-6- yl]-[(3S)-3- (1H-triazol-5- yl)pyrrolidin-1- yl]methanone | 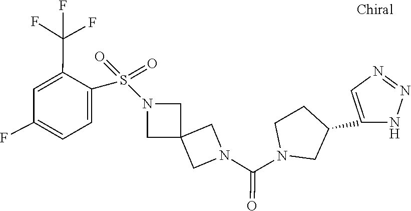 [SMILES: O=C(N1CC[C@H](c2cnn[nH]2)C1)N1CC2(C1)CN(S(=O)(=O)c1ccc(F)cc1C(F)(F)F)C2] | 2-[4-Fluoro-2- (trifluoromethyl) phenyl]sulfonyl-2,6- diazaspiro[3.3] heptane;2,2,2- trifluoroacetic acid (CAS RN 2428639- 75-0) and 5-[(3S)- Pyrrolidin-3-yl]- 1H-triazole (CAS RN 2166205-35-0) HPLC YMC | 489.3 [M + H]⁺ |
| 235 | [3-[[2-Fluoro-4- (trifluoromethyl) phenyl]methoxy] azetidin-1-yl]- [(3S)-3-(4H- 1,2,4-triazol-3- yl)pyrrolidin-1- yl]methanone | 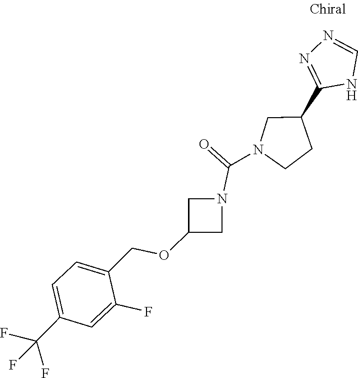 [SMILES: O=C(N1CC(OCc2ccc(C(F)(F)F)cc2F)C1)N1CC[C@H](c2nnc[nH]2)C1] | 3-((2-Fluoro-4- (trifluoromethyl) benzyl)oxy) azetidine; 4- methylbenzene- sulfonic acid (CAS RN 2135785-94-1) and 3-[(3 S)-Pyrrolidin- 3-yl]-4H-1,2,4- triazole (CAS RN 2165724-79-6) | 414.3 |
| 254 | 1-[3-[4-[3-(2,2,2- Trifluoroethoxy) azetidin-1- yl]phenyl] azetidine-1- carbonyl] pyrrolidine-3- carboxamide | 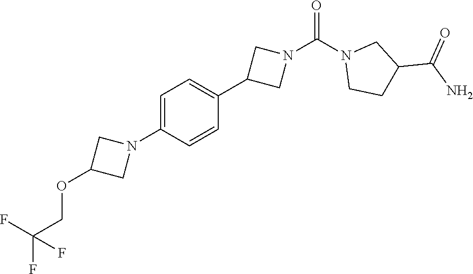 [SMILES: NC(=O)C1CCN(C(=O)N2CC(c3ccc(N4CC(OCC(F)(F)F)C4)cc3)C2)C1] | 1-[4-(Azetidin-3- yl)phenyl]-3-(2,2,2- trifluoroethoxy) azetidine; 4- methylbenzene- sulfonic acid (BB58) and Pyrrolidine-3- carboxamide;hydro chloride ( CAS RN 644972-57-6) HPLC YMC | 427.3 [M + H]⁺ |
| 261 | [3-[4-(4-Chloro- 2-methylsulfonyl- phenyl)phenyl] azetidin-1-yl]- [(3S)-3-(1,2,4- triazol-4- yl)pyrrolidin-1- yl]methanone | 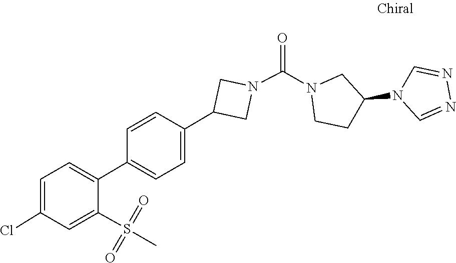 [SMILES: CS(=O)(=O)c1cc(Cl)ccc1-c1ccc(C2CN(C(=O)N3CC[C@H](n4cnnc4)C3)C2)cc1] | 3-[4-(4-Chloro-2- methylsulfonyl- phenyl)phenyl] azetidine;4- methylbenzene- sulfonic acid (BB44) and 4-(3S)-3- Pyrrolidinyl-4H- 1,2,4-triazole (CAS RN1234191-68-4) | 486.3 [M + H]⁺ |
| 274 | (3S)-1-[3-[[2- Fluoro-4- (trifluoromethyl) phenyl]methoxy] azetidine-1- carbonyl] pyrrolidine-3- carboxamide | 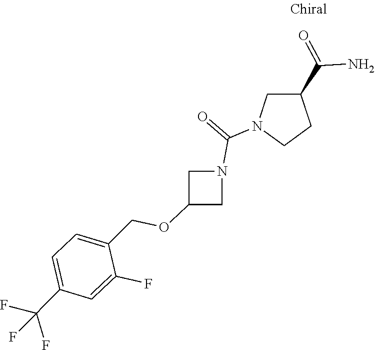 [SMILES: NC(=O)[C@H]1CCN(C(=O)N2CC(OCc3ccc(C(F)(F)F)cc3F)C2)C1] | 3-((2-Fluoro-4- (trifluoromethyl) benzyl)oxy) azetidine; 4- methylbenzene- sulfonic acid (CAS RN 2135785-94-1) and (3S)-Pyrrolidine-3- carboxamide ( CAS RN 573704-64-0) | 390.3 [M + H]⁺ |
| 286 | [3-[[2-Fluoro-4- (trifluoromethyl) phenyl] methylamino] azetidin-1-yl]-[3- (4H-1,2,4-triazol- 3-yl)pyrrolidin-1- yl]methanone | 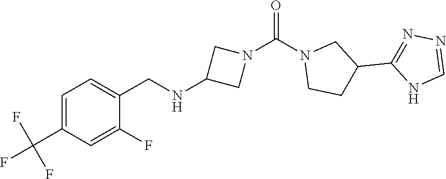 [SMILES: O=C(N1CC(NCc2ccc(C(F)(F)F)cc2F)C1)N1CCC(c2nnc[nH]2)C1] | N-[[2-Fluoro-4- (trifluoromethyl) phenyl]methyl] azetidin-3-amine;4- methylbenzene- sulfonic acid (BB99) and 3-Pyrrolidin-3- yl-4H-1,2,4- triazole;dihydro- chloride (CAS RN 1909319-47-6) | 413.3 [M + H]⁺ |
| 292 | [(3S)-3-(1,2,4- Triazol-4- yl)pyrrolidin-1- yl]-[3-[4-[[1- (trifluoromethyl) cyclopropyl] methylamino] phenyl]azetidin- 1-yl]methanone | 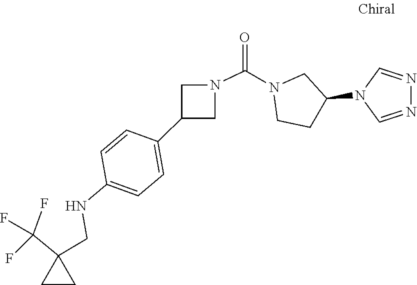 [SMILES: O=C(N1CC(c2ccc(NCC3(C(F)(F)F)CC3)cc2)C1)N1CC[C@H](n2cnnc2)C1] | 4-(Azetidin-3-yl)- N-[[1- (trifluoromethyl) cyclopropyl] methyl] aniline;4- methylbenzene- sulfonic acid (BB80) and 4-(3S)-3- Pyrrolidinyl-4H- 1,2,4-triazole (CAS RN1234191-68-4) | 435.4 [M + H]⁺ |
| 296 | 1-[3-[4-(4-Chloro- 2-methylsulfonyl- phenyl)phenyl] azetidine-1- carbonyl]- N-methyl- pyrrolidine-3- carboxamide | 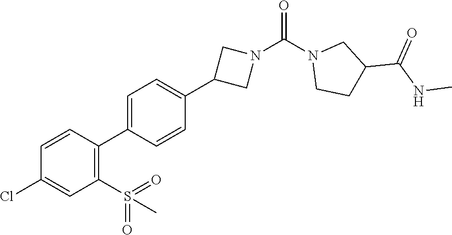 [SMILES: CNC(=O)C1CCN(C(=O)N2CC(c3ccc(-c4ccc(Cl)cc4S(C)(=O)=O)cc3)C2)C1] | 3-[4-(4-Chloro-2- methylsulfonyl- phenyl)phenyl] azetidine;4- methylbenzene- sulfonic acid (BB44) and N- Methylpyrrolidine- 3-carboxamide (CAS RN 1007870- 02-1) HPLC YMC | 476.2 [M + H]⁺ |
| 298 | (3S)-1-[3-[4-[[1- (Trifluoromethyl) cyclopropyl]amino] phenyl]azetidine-1- carbonyl] pyrrolidine-3- carboxamide | 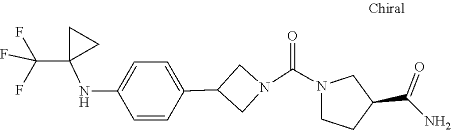 [SMILES: NC(=O)[C@H]1CCN(C(=O)N2CC(c3ccc(NC4(C(F)(F)F)CC4)cc3)C2)C1] | 4-(Azetidin-3-yl)- N-[1- (trifluoromethyl) cyclopropyl]aniline; 4-methylbenzene- sulfonic acid (BB104) and (3S)- Pyrrolidine-3- | 397.3 [M + H]⁺ |
| | | | carboxamide ( CAS | |
| | | | RN 573704-64-0) | |
| 299 | (3S)-1-[3-[4-[[1- (Trifluoromethyl) cyclopropyl] methylamino] phenyl]azetidine- 1-carbonyl] pyrrolidine-3- carboxamide | 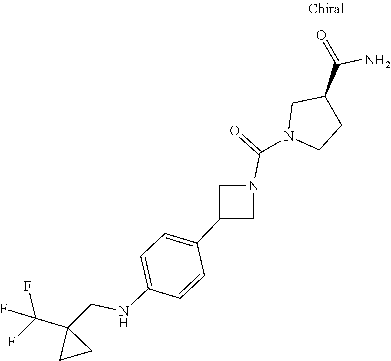 [SMILES: NC(=O)[C@H]1CCN(C(=O)N2CC(c3ccc(NCC4(C(F)(F)F)CC4)cc3)C2)C1] | 4-(Azetidin-3-yl)- N-[[1- (trifluoromethyl) cyclopropyl]methyl] aniline;4- methylbenzene- sulfonic acid (BB80) and (3S)- Pyrrolidine-3- carboxamide ( CAS RN 573704-64-0) | 411.3 [M + H]⁺ |

[M + H]⁺

| | | | | |
|---|---|---|---|---|
| 316 | [3-[4-[3-(2,2- Dimethylpropyl) triazol-4- yl]phenyl]azetidin- 1-yl]-[(3S)-3- (tetrazol-1- yl)pyrrolidin-1- yl]methanone | 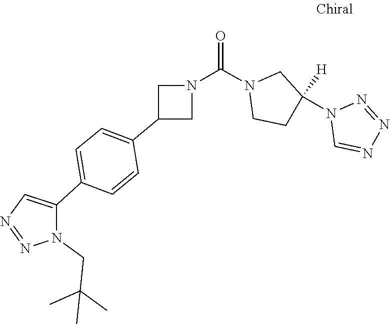 [SMILES: CC(C)(C)Cn1nncc1-c1ccc(C2CN(C(=O)N3CC[C@H](n4cnnn4)C3)C2)cc1] | 5-[4-(Azetidin-3- yl)phenyl]-1-(2,2- dimethylpropyl) triazole, 4- methylbenzene- sulfonic acid (BB45) and 1-[(3S)- Pyrrolidin-3- yl]tetrazole (BB86) | 436.3 [M + H]⁺ |
| 318 | [3-[5-(2,4- Dichlorophenoxy) pyrazin-2- yl]azetidin-1-yl]- [(3R)-3-(tetrazol- 1-yl)pyrrolidin-1- yl]methanone | 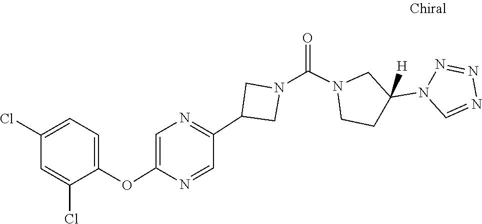 [SMILES: O=C(N1CC(c2cnc(Oc3ccc(Cl)cc3Cl)cn2)C1)N1CC[C@@H](n2cnnn2)C1] | 2-(Azetidin-3-yl)- 5-(2,4- dichlorophenoxy) pyrazine, 4- methylbenzenesulf onic acid (BB88) and 1-[(3R)- Pyrrolidin-3- yl]tetrazole (BB87) | 461.3 [M + H]⁺ |
| 322 | [3-[4-(4- Cyclopropyl- pyrimidin-2- yl)oxyphenyl] azetidin-1-yl]- [(3S)-3- (tetrazol-1- yl)pyrrolidin-1- yl]methanone | 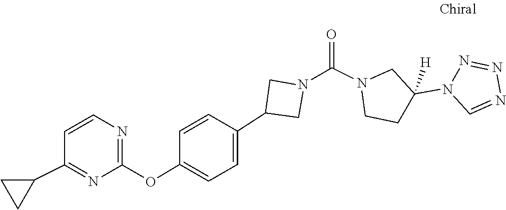 [SMILES: O=C(N1CC(c2ccc(Oc3nccc(C4CC4)n3)cc2)C1)N1CC[C@H](n2cnnn2)C1] | 2-[4-(Azetidin-3- yl)phenoxy]-4- cyclopropyl- pyrimidine;4- methylbenzene- sulfonic acid (BB91) and 1-[(3S)- Pyrrolidin-3- yl]tetrazole (BB86) | 433.2 [M + H]⁺ |
| 323 | [3-[5-(2,4- Dichlorophenoxy) pyrazin-2- yl]azetidin-1-yl]- [(3S)-3-(tetrazol- 1-yl)pyrrolidin-1- yl]methanone | 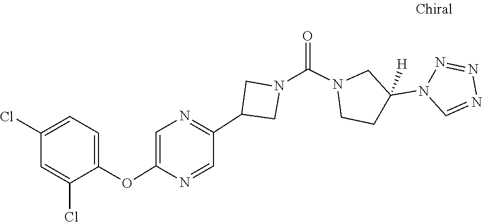 [SMILES: O=C(N1CC(c2cnc(Oc3ccc(Cl)cc3Cl)cn2)C1)N1CC[C@H](n2cnnn2)C1] | 2-(Azetidin-3-yl)- 5-(2,4- dichlorophenoxy) pyrazine;4- methylbenzene- sulfonic acid (BB88) and 1-[(3S)- Pyrrolidin-3- yl]tetrazole (BB86) | 461.3 [M + H]⁺ |
| 332 | (3S)-1-[6-[[5- (Trifluoromethyl) pyrazin-2- yl]methyl]-2- azaspiro[3.3] heptane-2- carbonyl] pyrrolidine-3- carboxamide | 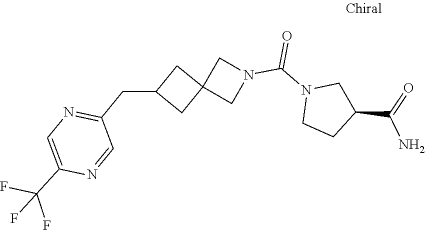 [SMILES: NC(=O)[C@H]1CCN(C(=O)N2CC3(CC(Cc4cnc(C(F)(F)F)cn4)C3)C2)C1] | 6-[[5- (Trifluoromethyl) pyrazin-2- yl]methyl]-2- azaspiro[3.3] heptane ;4- methylbenzene- sulfonic acid (BB89) and (3S)- Pyrrolidine-3- carboxamide ( CAS RN 573704-64-0) HPLC YMC | 398.2 [M + H]⁺ |
| 333 | [7-[[2-Fluoro-4- (trifluoromethyl) phenyl]methyl]- 2,7- diazaspiro[3.5] nonan-2-yl]-[3- (4H-1,2,4-triazol- 3-yl)pyrrolidin-1- yl]methanone | 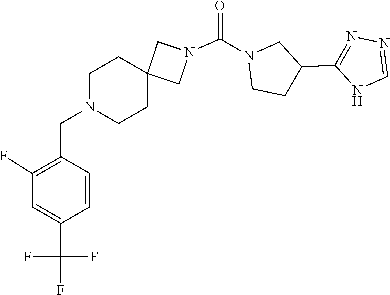 [SMILES: O=C(N1CCC(c2nnc[nH]2)C1)N1CC2(CCN(Cc3ccc(C(F)(F)F)cc3F)CC2)C1] | 7-[[2-Fluoro-4- (trifluoromethyl) phenyl]methyl]-2,7- diazaspiro[3.5] nonane; 4- methylbenzene- sulfonic acid (BB93) and 3-Pyrrolidin-3- yl-4H-1,2,4- triazole;dihydro- chloride (CAS RN 1909319-47-6) HPLC YMC | 467.4 [M + H]⁺ |
| 334 | [7-[2-Fluoro-4- (trifluoromethyl) phenoxy]-2- azaspiro[3.5] nonan-2-yl]-[3- (4H-1,2,4- triazol-3- yl)pyrrolidin-1- yl]methanone | 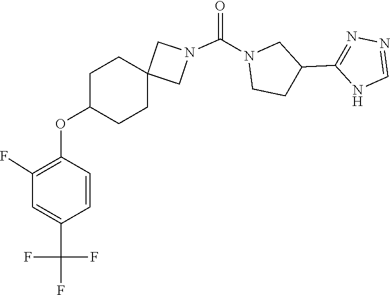 [SMILES: O=C(N1CCC(c2nnc[nH]2)C1)N1CC2(CCC(Oc3ccc(C(F)(F)F)cc3F)CC2)C1] | 7-[2-Fluoro-4- (trifluoromethyl) phenoxy]-2- azaspiro[3.5] nonane;4- methylbenzene- sulfonic acid (BB81) and 3-Pyrrolidin-3- yl-4H-1,2,4- triazole;dihydro- chloride (CAS RN 1909319-47-6) HPLC YMC | 468.3 [M + H]⁺ |
| 335 | [6-[2-Fluoro-4- (trifluoromethyl) anilino]-2- azaspiro[3.3] heptan-2-yl]-[3- (4H-1,2,4- triazol-3- yl)pyrrolidin-1- yl]methanone | 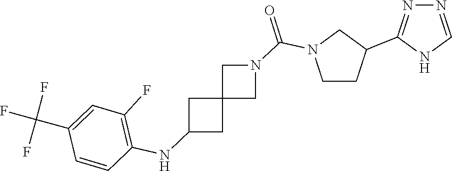 [SMILES: O=C(N1CCC(c2nnc[nH]2)C1)N1CC2(CC(Nc3ccc(C(F)(F)F)cc3F)C2)C1] | N-[2-Fluoro-4- (trifluoromethyl) phenyl]-2- azaspiro[3.3]heptan- 6-amine; 4- methylbenzene- sulfonic acid (BB100) and 3-Pyrrolidin-3- yl-4H-1,2,4- triazole;dihydro- chloride (CAS RN | 439.3 [M + H]⁺ |
| | | | 1909319-47-6) | |
| | | | HPLC YMC | |
| 344 | [(3S)-3-(1H-1,2,4- Triazol-5- yl)pyrrolidin- 1-yl]-[3-[4-[[1- (trifluoromethyl) cyclopropyl] amino]phenyl] azetidin-1- yl]methanone | 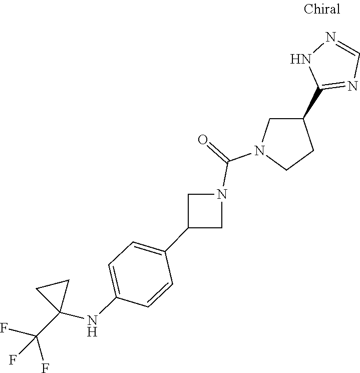 [SMILES: O=C(N1CC(c2ccc(NC3(C(F)(F)F)CC3)cc2)C1)N1CC[C@H](c2ncn[nH]2)C1] | 4-(Azetidin-3-yl)- N-[1- (trifluoromethyl) cyclopropyl] aniline;4- methylbenzene- sulfonic acid (BB104) and 5-[(3S)- Pyrrolidin-3-yl]- 1H-1,2,4-triazole (CAS RN 2165724- 79-6) HPLC YMC | 421.3 [M + H]⁺ |
| 359 | 1-[3-[4-(4-Chloro- 2-methylsulfonyl- phenyl)phenyl] azetidine-1- carbonyl] pyrrolidine-3- sulfonamide | 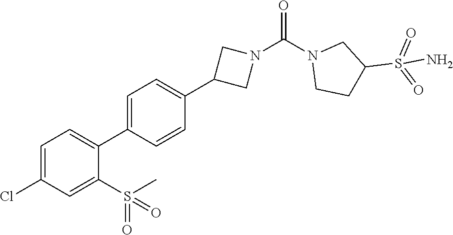 [SMILES: CS(=O)(=O)c1cc(Cl)ccc1-c1ccc(C2CN(C(=O)N3CCC(S(N)(=O)=O)C3)C2)cc1] | 3-[4-(4-Chloro-2- methylsulfonyl- phenyl)phenyl] azetidine;4- methylbenzene- sulfonic acid (BB44) and Pyrrolidine-3- sulfonamide ( CAS RN 1208507-46-3) HPLC YMC | 498.2 [M + H]⁺ |
| 361 | (3S)-1-[3-[5-(2- Chloro-4- methylsulfonyl- phenyl)-2- pyridyl]azetidine- 1-carbonyl] pyrrolidine- 3-carboxamide | 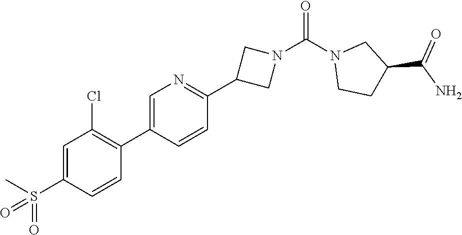 [SMILES: CS(=O)(=O)c1ccc(-c2ccc(C3CN(C(=O)N4CC[C@H](C(N)=O)C4)C3)nc2)c(Cl)c1] | 2-(Azetidin-3-yl)- 5-(2-chloro-4- methylsulfonyl- phenyl)pyridine;4- methylbenzene- sulfonic acid (BB62) and (3S)- Pyrrolidine-3- carboxamide ( CAS RN 573704-64-0) HPLC YMC | 463.2 [M + H]⁺ |
| 366 | (3S)-1-[3-[5-(4- Chloro-2- methylsulfonyl- phenyl)-2- pyridyl]azetidine- 1-carbonyl] pyrrolidine-3- carboxamide | 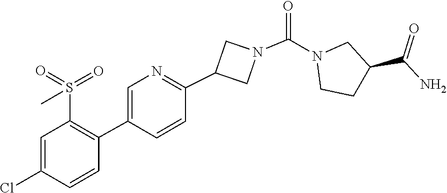 [SMILES: CS(=O)(=O)c1cc(Cl)ccc1-c1ccc(C2CN(C(=O)N3CC[C@H](C(N)=O)C3)C2)nc1] | 2-(Azetidin-3-yl)- 5-(4-chloro-2- methylsulfonyl- phenyl)pyridine; 4- methylbenzene- sulfonic acid (BB63) and (3S)- Pyrrolidine-3- carboxamide ( CAS RN 573704-64-0) HPLC YMC | 463.1 [M + H]⁺ |
| 371 | [3-[[2-Fluoro-4- (trifluoromethyl) phenyl] methylamino] azetidin-1-yl]- [(3S)-3-(1H- 1,2,4-triazol-5- yl)pyrrolidin-1- yl]methanone | 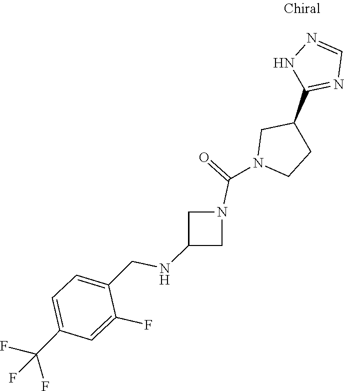 [SMILES: O=C(N1CC(NCc2ccc(C(F)(F)F)cc2F)C1)N1CC[C@H](c2ncn[nH]2)C1] | N-[[2-Fluoro-4- (trifluoromethyl) phenyl]methyl] azetidin-3-amine; 4- methylbenzene- sulfonic acid (BB99) and 5-[(3S)- Pyrrolidin-3-yl]- 1H-1,2,4-triazole (CAS RN 2165724- 79-6) HPLC YMC | 413.2 [M + H]⁺ |
| 377 | [(3S)-3-(Tetrazol- 1-yl)pyrrolidin-1- yl]-[3-[4-[4- (trifluoromethyl) pyrimidin-2- yl]oxyphenyl] azetidin-1- yl]methanone | 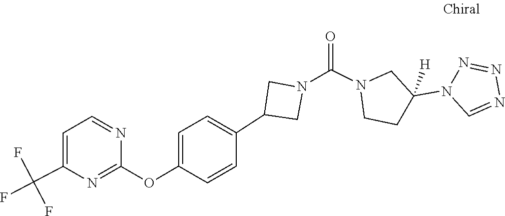 [SMILES: O=C(N1CC(c2ccc(Oc3nccc(C(F)(F)F)n3)cc2)C1)N1CC[C@H](n2cnnn2)C1] | 2-[4-(Azetidin-3- yl)phenoxy]-5- (trifluoromethyl) pyrazine;4- methylbenzene- sulfonic acid (BB90) and 1-[(3S)- pyrrolidin-3- yl]tetrazole (BB86) HPLC YMC | 461.2 [M + H]⁺ |
| 387 | [3-[4-(4-Chloro- 2-methylsulfonyl- phenyl)phenyl] azetidin-1-yl]- [(3S)-3- (tetrazol-1- yl)pyrrolidin-1- yl]methanone | 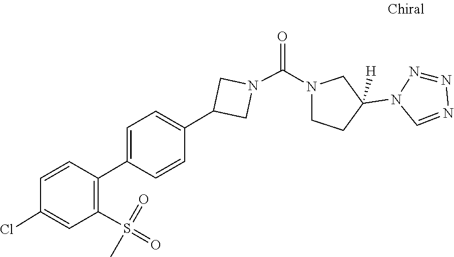 [SMILES: CS(=O)(=O)c1cc(Cl)ccc1-c1ccc(C2CN(C(=O)N3CC[C@H](n4cnnn4)C3)C2)cc1] | 3-[4-(4-Chloro-2- methylsulfonyl- phenyl)phenyl] azetidine;4- methylbenzene- sulfonic acid (BB44) and 1-[(3S)- pyrrolidin-3- yl]tetrazole (BB86) | 487.2 [M + H]⁺ |
| 389 | [7-[2-Fluoro-4- (trifluoromethyl) phenoxy]-2- azaspiro[3.5] nonan- 2-yl]-[(3S)-3- (1,2,4-triazol-4- yl)pyrrolidin-1- yl]methanone | 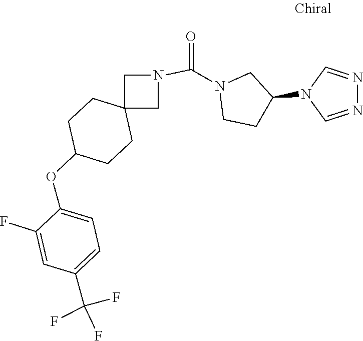 [SMILES: O=C(N1CC[C@H](n2cnnc2)C1)N1CC2(CCC(Oc3ccc(C(F)(F)F)cc3F)CC2)C1] | 7-[2-Fluoro-4- (trifluoromethyl) phenoxy]-2- azaspiro[3.5] nonane;4- methylbenzene- sulfonic acid (BB81) and 4-[(3S)- Pyrrolidin-3-yl]- 1,2,4-triazole (CAS RN 1234191-68-4) | 468.4 [M + H]⁺ |
| 390 | (3S)-1-[3-[4-(2- Chloro-4- methylsulfonyl- phenyl)phenyl] azetidine-1- carbonyl] pyrrolidine-3- carboxamide | 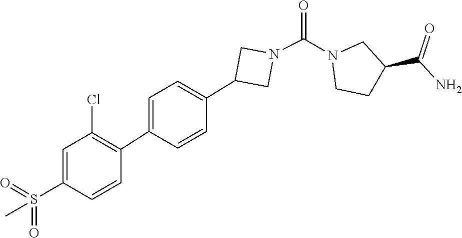 [SMILES: CS(=O)(=O)c1ccc(-c2ccc(C3CN(C(=O)N4CC[C@H](C(N)=O)C4)C3)cc2)c(Cl)c1] | 3-[4-(2-Chloro-4- methylsulfonyl- phenyl)phenyl] azetidine; 4- methylbenzene- sulfonic acid (BB50) and (3S)- Pyrrolidine-3- carboxamide ( CAS RN 573704-64-0) Precipitation, filtered | 462.2 [M + H]⁺ |
| 393 | (3S)-1-[3-[4-(6- Chloro-4- methylsulfonyl- 3-pyridyl)phenyl] azetidine-1- carbonyl] pyrrolidine- 3-carboxamide | 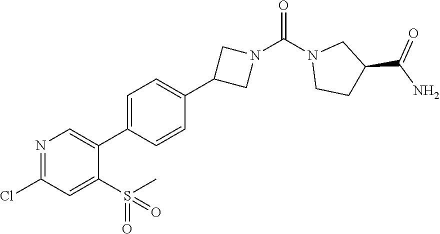 [SMILES: CS(=O)(=O)c1cc(Cl)ncc1-c1ccc(C2CN(C(=O)N3CC[C@H](C(N)=O)C3)C2)cc1] | 5-[4-(Azetidin-3- yl)phenyl]-2- chloro-4- methylsulfonyl- pyridine;4- methylbenzene- sulfonic acid (BB82) and (3S)- Pyrrolidine-3- carboxamide ( CAS RN 573704-64-0) | 463.2 [M + H]⁺ |
| 394 | [3-[4-(4-Chloro-2- methylsulfonyl- phenyl)phenyl] azetidin-1- yl]-[(3S)-3- (1H-1,2,4-triazol- 5-yl)pyrrolidin-1- yl]methanone | 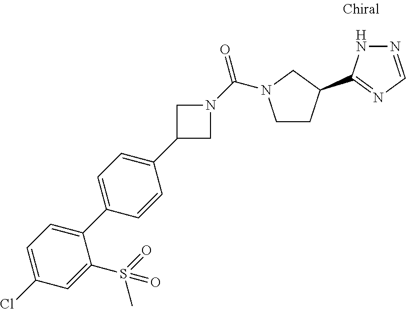 [SMILES: CS(=O)(=O)c1cc(Cl)ccc1-c1ccc(C2CN(C(=O)N3CC[C@H](c4ncn[nH]4)C3)C2)cc1] | 3-[4-(4-Chloro-2- methylsulfonyl- phenyl)phenyl] azetidine;4- methylbenzene- sulfonic acid (BB44) and 5-[(3S)- Pyrrolidin-3-yl]- 1H-1,2,4-triazole (CAS RN 2165724- 79-6) HPLC YMC | 486.2 [M + H]⁺ |
| 400 | [7-[[2-Fluoro-4- (trifluoromethyl) phenyl]methyl]- 2,7- diazaspiro[3.5] nonan-2-yl]- [(3S)-3-(4H- 1,2,4-triazol-3- yl)pyrrolidin-1- yl]methanone | 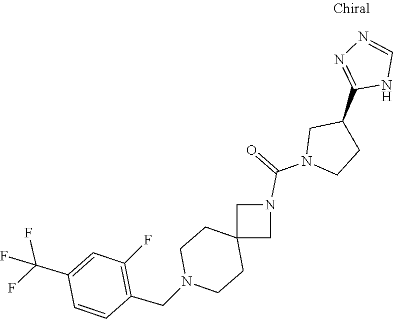 [SMILES: O=C(N1CC[C@H](c2nnc[nH]2)C1)N1CC2(CCN(Cc3ccc(C(F)(F)F)cc3F)CC2)C1] | 7-[[2-Fluoro-4- (trifluoromethyl) phenyl]methyl]-2,7- diazaspiro[3.5] nonane; 4- methylbenzene- sulfonic acid (BB93) and 3-[(3S)- Pyrrolidin-3-yl]- 4H-1,2,4-triazole (CAS RN 2165724- 79-6) | 467.3 [M + H]⁺ |
| 402 | [7-[2-Fluoro-4- (trifluoromethyl) phenoxy]-2- azaspiro[3.5] nonan-2-yl]- [(3S)-3-(4H- 1,2,4-triazol-3- yl)pyrrolidin-1- yl]methanone | 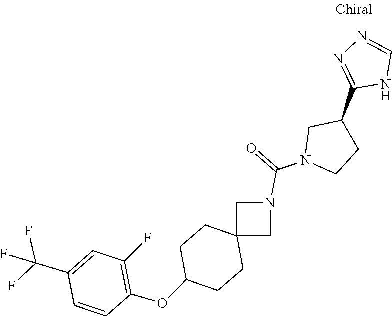 [SMILES: O=C(N1CC[C@H](c2nnc[nH]2)C1)N1CC2(CCC(Oc3ccc(C(F)(F)F)cc3F)CC2)C1] | 7-[2-Fluoro-4- (trifluoromethyl) phenoxy]-2- azaspiro[3.5] nonane;4- methylbenzene- sulfonic acid (BB81) and 3-[(3S)- Pyrrolidin-3-yl]- 4H-1,2,4-triazole (CAS RN 2165724- 79-6) HPLC YMC | 468.2 [M + H]⁺ |
| 405 | (3S)-1-[3-[5-(4- Chloro-2-fluoro- phenyl)-2- pyridyl]azetidine- 1-carbonyl] pyrrolidine-3- carboxamide | 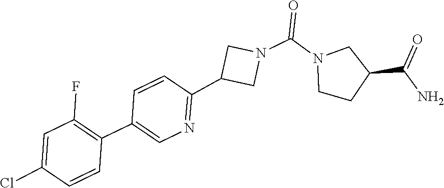 [SMILES: NC(=O)[C@H]1CCN(C(=O)N2CC(c3ccc(-c4ccc(Cl)cc4F)cn3)C2)C1] | 2-(Azetidin-3-yl)- 5-(4-chloro-2- fluoro- phenyl)pyridine;4- methylbenzene- sulfonic acid (BB66) and (3S)- Pyrrolidine-3- carboxamide ( CAS RN 573704-64-0) Precipitation, filtered | 403.1 [M + H]⁺ |
| 406 | (3S)-1-[3-[5-(2,4- Dichlorophenyl)-2- pyridyl]azetidine- 1-carbonyl] pyrrolidine-3- carboxamide | 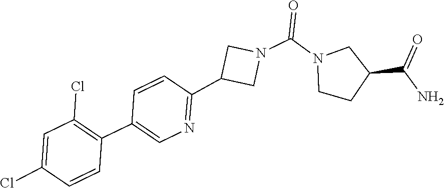 [SMILES: NC(=O)[C@H]1CCN(C(=O)N2CC(c3ccc(-c4ccc(Cl)cc4Cl)cn3)C2)C1] | 2-(Azetidin-3-yl)- 5-(2,4- dichlorophenyl) pyridine; 4- methylbenzene- sulfonic acid (BB67) and (3S)- Pyrrolidine-3- carboxamide ( CAS RN 573704-64-0) | 419.1 [M + H]⁺ |
| 407 | (3S)-1-[3-[4-(5- Chloro-3- methylsulfonyl-2- pyridyl)phenyl] azetidine-1- carbonyl] pyrrolidine-3- carboxamide | 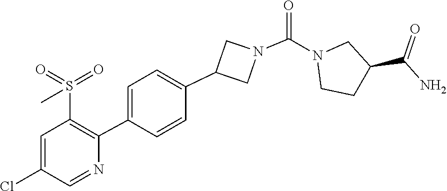 [SMILES: CS(=O)(=O)c1cc(Cl)cnc1-c1ccc(C2CN(C(=O)N3CC[C@H](C(N)=O)C3)C2)cc1] | 2-[4-(Azetidin-3- yl)phenyl]-5- chloro-3- methylsulfonyl- pyridine; 4- methylbenzene- sulfonic acid (BB65) and (3S)- Pyrrolidine-3- carboxamide (CAS RN 573704-64-0) | 463.1 [M + H]⁺ |
| 411 | (3S)-1-[3-[4-[5- (Trifluoromethyl) pyrazin-2- yl]phenyl] azetidine-1- carbonyl] pyrrolidine-3- carboxamide | 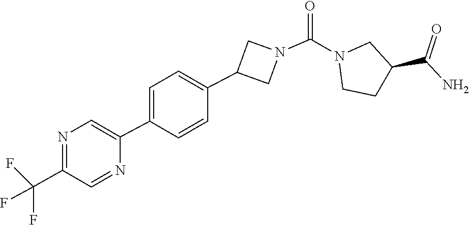 [SMILES: NC(=O)[C@H]1CCN(C(=O)N2CC(c3ccc(-c4cnc(C(F)(F)F)cn4)cc3)C2)C1] | 2-[4-(Azetidin-3- yl)phenyl]-5- (trifluoromethyl) pyrazine; 4- methylbenzene- sulfonic acid (BB69) and (3S)- Pyrrolidine-3- carboxamide ( CAS RN 573704-64-0) | 420.1 [M + H]⁺ |
| 413 | (4H-1,2,4-triazol-3- [2-[[2-Fluoro-4- (trifluoromethyl) phenyl]methyl]- 2,7-diazaspiro [3.5]nonan-7- yl]-[(3S)-3- yl)pyrrolidin-1- yl]methanone | 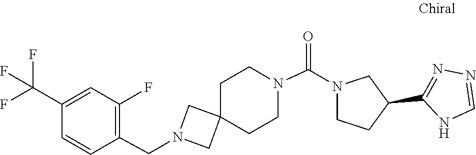 [SMILES: O=C(N1CCC2(CC1)CN(Cc1ccc(C(F)(F)F)cc1F)C2)N1CC[C@H](c2nnc[nH]2)C1] | 2-[[2-Fluoro-4- (trifluoromethyl) phenyl]methyl]-2,7- diazaspiro[3.5] nonane (CAS RN 2428638-82-6) and 3-[(3S)-Pyrrolidin- 3-yl]-4H-1,2,4- triazole (CAS RN 2165724-79-6) | 467.3 [M + H]⁺ |
| 415 | (3S)-1-[7-[[2- Fluoro-4- (trifluoromethyl) phenyl]methyl]- 2,7-diazaspiro [3.5]nonane-2- carbonyl] pyrrolidine-3- carboxamide | 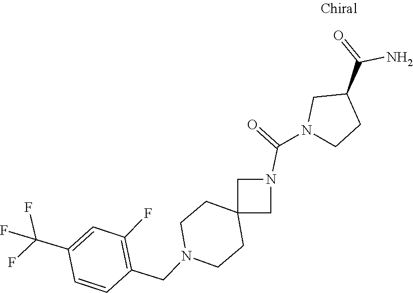 [SMILES: NC(=O)[C@H]1CCN(C(=O)N2CC3(CCN(Cc4ccc(C(F)(F)F)cc4F)CC3)C2)C1] | 7-[[2-Fluoro-4- (trifluoromethyl) phenyl]methyl]- 2,7-diazaspiro [3.5]nonane; 4- methylbenzene- sulfonic acid (BB93) and (3S)- Pyrrolidine-3- carboxamide ( CAS RN 573704-64-0) | 443.3 [M + H]⁺ |
| 417 | (3S)-1-[7-[2- Fluoro-4- (trifluoromethyl) phenoxy]-2- azaspiro[3.5] nonane-2- carbonyl] pyrrolidine-3- carboxamide | 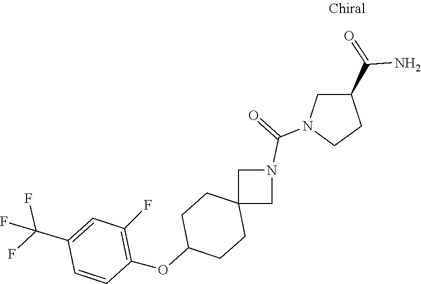 [SMILES: NC(=O)[C@H]1CCN(C(=O)N2CC3(CCC(Oc4ccc(C(F)(F)F)cc4F)CC3)C2)C1] | 7-[2-Fluoro-4- (trifluoromethyl) phenoxy]-2- azaspiro[3.5] nonane;4- methylbenzene- sulfonic acid (BB81) and (3S)- Pyrrolidine-3- carboxamide ( CAS RN 573704-64-0) HPLC YMC | 444.2 [M + H]⁺ |
| 424 | [(3S)-3-(Tetrazol- 1-yl)pyrrolidin-1- yl]-[6-[[6- (trifluoromethyl)- 3-pyridyl] methyl]-2- azaspiro[3.3] heptan-2- yl]methanone | 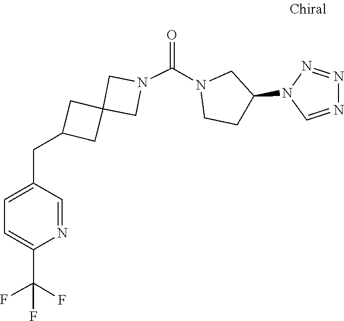 [SMILES: O=C(N1CC[C@H](n2cnnn2)C1)N1CC2(CC(Cc3ccc(C(F)(F)F)nc3)C2)C1] | 6-[[6- (trifluoromethyl)-3- pyridyl]methyl]-2- azaspiro[3.3] heptane; 4- methylbenzene- sulfonic acid (BB73) and 1-[(3S)- pyrrolidin-3- yl]tetrazole (BB86) HPLC YMC | 422.2 [M + H]⁺ |
| 427 | [(3R)-3-(Tetrazol- 1-yl)pyrrolidin-1- yl]-[6-[6- (trifluoromethyl)-3- pyridyl]methyl]-2- azaspiro[3.3] heptan-2- yl]methanone | 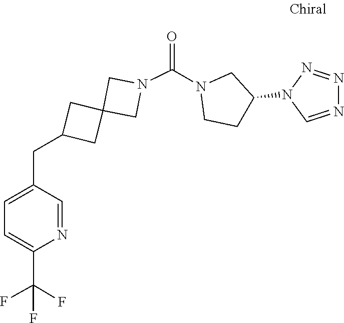 [SMILES: O=C(N1CC[C@@H](n2cnnn2)C1)N1CC2(CC(Cc3ccc(C(F)(F)F)nc3)C2)C1] | 6-[[6- (trifluoromethyl)-3- pyridyl]methyl]-2- azaspiro[3.3] heptane; 4- methylbenzene- sulfonic acid (BB73) and 1-[(3R)- pyrrolidin-3- yl]tetrazole (BB87) HPLC YMC | 422.2 [M + H]⁺ |
| 435 | [(3S)-3-(1H-1,2,4- Triazol-5- yl)pyrrolidin-1-yl]- [6-[[5- (trifluoromethyl)-2- pyridyl]methyl]-2- azaspiro[3.3] heptan-2- yl]methanone | 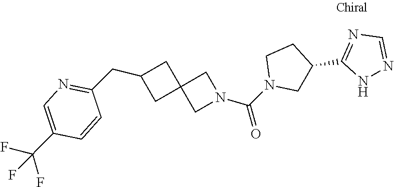 [SMILES: O=C(N1CC[C@H](c2ncn[nH]2)C1)N1CC2(CC(Cc3ccc(C(F)(F)F)cn3)C2)C1] | 4-Methylbenzene- sulfonic acid; 6-[[5- (trifluoromethyl)-2- pyridyl]methyl]-2- azaspiro[3.3] heptane (CAS RN 2855958-37-9) and 5-[(3S)-Pyrrolidin- 3-yl]-1H-1,2,4- triazole (CAS RN 2165724-79-6) HPLC YMC | 421.2 [M + H]⁺ |
| 445 | [3-[5-(4-Chloro-2- methylsulfonyl- phenyl)-2- pyridyl]azetidin-1- yl]-[(3R)-3- hydroxypyrrolidin- 1-yl]methanone | 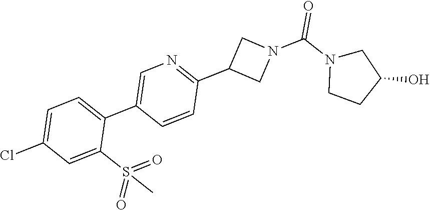 [SMILES: CS(=O)(=O)c1cc(Cl)ccc1-c1ccc(C2CN(C(=O)N3CC[C@@H](O)C3)C2)nc1] | 2-(Azetidin-3-yl)- 5-(4-chloro-2- methylsulfonyl- phenyl)pyridine; 4- methylbenzene- sulfonic acid (BB63) and R-3- pyrrolidinol ( CAS RN 573704-64-0) HPLC YMC | 436.2 [M + H]⁺ |
| 454 | [(3S)-3-(1,2,4- Triazol-4- yl)pyrrolidin-1-yl]- [3-[6-[[1- (trifluoromethyl) cyclopropyl] methylamino]-3- pyridyl]azetidin-1- yl]methanone | 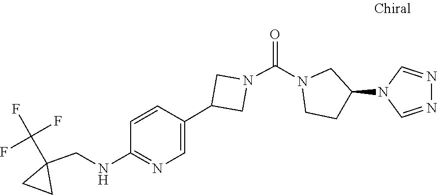 [SMILES: O=C(N1CC(c2ccc(NCC3(C(F)(F)F)CC3)nc2)C1)N1CC[C@H](n2cnnc2)C1] | 5-(Azetidin-3-yl)- N-[[1- (trifluoromethyl) cyclopropyl]methyl] pyridin-2-amine;4- methylbenzene- sulfonic acid (BB83) and 4-[(3S)- Pyrrolidin-3-yl]- 1,2,4-triazole (CAS RN 1234191-68-4) | 436.4 [M + H]⁺ |
| 468 | (3S)-1-[3-[6-[[1- (Trifluoromethyl) cyclopropyl] methylamino]-3- pyridyl]azetidine-1- carbonyl] pyrrolidine-3- carboxamide | 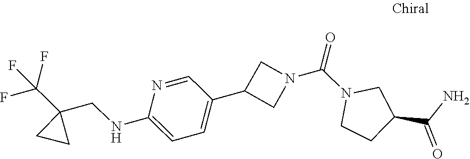 [SMILES: NC(=O)[C@H]1CCN(C(=O)N2CC(c3ccc(NCC4(C(F)(F)F)CC4)nc3)C2)C1] | 5-(Azetidin-3-yl)- N-[[1- (trifluoromethyl) cyclopropyl]methyl] pyridin-2-amine;4- methylbenzene- sulfonic acid (BB83) and (3S)- Pyrrolidine-3- carboxamide ( CAS RN 573704-64-0) | 412.3 [M + H]⁺ |
| 474 | (3S)-1-[7-[6- (Trifluoromethyl) pyridazin-3- yl]oxy-2- azaspiro[3.5] nonane-2- carbonyl] pyrrolidine-3- carboxamide | 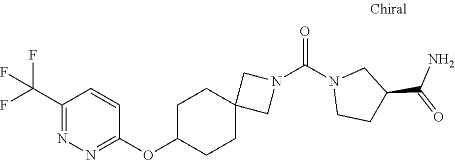 [SMILES: NC(=O)[C@H]1CCN(C(=O)N2CC3(CCC(Oc4ccc(C(F)(F)F)nn4)CC3)C2)C1] | 7-[6- (Trifluoromethyl) pyridazin-3-yl]oxy- 2-azaspiro[3.5] nonane; 4- methylbenzene- sulfonic acid (BB105) and (3S)- Pyrrolidine-3- carboxamide ( CAS RN 573704-64-0) HPLC YMC | 428.4 [M + H]⁺ |
| 521 | (3S)-1-[7-[5- (Trifluoromethyl) pyrimidin-2-yl] oxy-2- azaspiro[3.5] nonane-2- carbonyl] pyrrolidine-3- carboxamide | 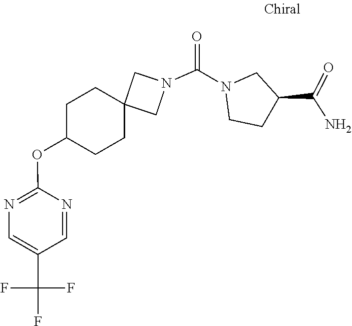 [SMILES: NC(=O)[C@H]1CCN(C(=O)N2CC3(CCC(Oc4ncc(C(F)(F)F)cn4)CC3)C2)C1] | 7-[5- (Trifluoromethyl) pyrimidin-2-yl] oxy-2-zaspiro[3.5] nonane; 4- methylbenzene- sulfonic acid (BB101) and (3S)- Pyrrolidine-3- carboxamide ( CAS RN 573704-64-0) HPLC YMC | 428.3 [M + H]⁺ |
| 529 | (3S)-1-[7-[5- (Trifluoromethyl) pyrazin-2-yl]oxy- 2-azaspiro[3.5] nonane-2- carbonyl] pyrrolidine-3- carboxamide | 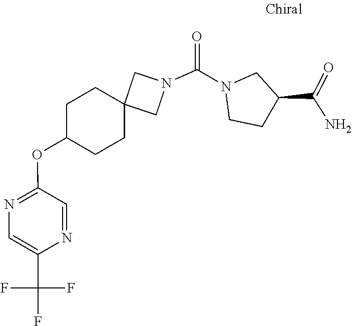 [SMILES: NC(=O)[C@H]1CCN(C(=O)N2CC3(CCC(Oc4cnc(C(F)(F)F)cn4)CC3)C2)C1] | 7-[5- (Trifluoromethyl) pyrazin-2-yl]oxy-2- azaspiro[3.5] nonane; 4- methylbenzene- sulfonic acid (BB102) and (3S)- Pyrrolidine-3- carboxamide ( CAS RN 573704-64-0) HPLC YMC | 428.3 [M + H]⁺ |
| 534 | [3-[[2-Chloro-4- (trifluoromethyl) phenyl] methylamino] azetidin-1-yl]- [(3S)-3-(1H-1,2,4- triazol-5- yl)pyrrolidin-1- yl]methanone | 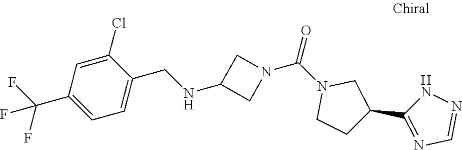 [SMILES: O=C(N1CC(NCc2ccc(C(F)(F)F)cc2Cl)C1)N1CC[C@H](c2ncn[nH]2)C1] | N-[[2-Chloro-4- (trifluoromethyl) phenyl]methyl] azetidin-3-amine;4- methylbenzene- sulfonic acid (CAS RN 2855958-94-8) and 5-[(3S)-Pyrrolidin- 3-yl]-1H-1,2,4- | 429.2 [M + H]⁺ |
| | | | triazole (CAS RN | |
| | | | 2165724-79-6) | |
| | | | HPLC YMC | |
| 544 | (3S)-1-[3-[6-(2- Azaspiro[3.4] octan-2-yl)-3- pyridyl]azetidine- 1-carbonyl] pyrrolidine-3- carboxamide | 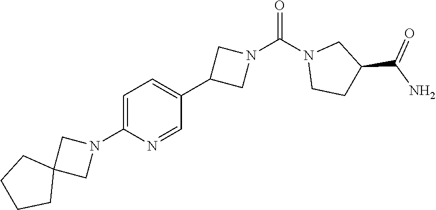 [SMILES: NC(=O)[C@H]1CCN(C(=O)N2CC(c3ccc(N4CC5(CCCC5)C4)nc3)C2)C1] | 2-[5-(Azetidin-3- yl)-2-pyridyl]-2- azaspiro[3.4] octane;4- methylbenzene- sulfonic acid (BB71) and (3S)- Pyrrolidine-3- carboxamide ( CAS RN 573704-64-0) HPLC YMC | 384.2 [M + H]⁺ |
| 548 | (3S)-1-[7-[6- (Trifluoromethyl)- 3-pyridyl]methyl]- 2-azaspiro[3.5] nonane-2- carbonyl] pyrrolidine-3- carboxamide | 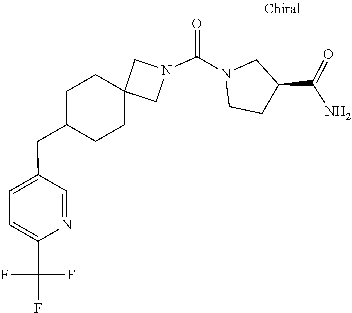 [SMILES: NC(=O)[C@H]1CCN(C(=O)N2CC3(CCC(Cc4ccc(C(F)(F)F)nc4)CC3)C2)C1] | 7-[[6- (Trifluoromethyl)- 3-pyridyl]methyl]- 2-azaspiro[3.5] nonane; 4- methylbenzene- sulfonic acid (BB97) and (3S)- Pyrrolidine-3- carboxamide ( CAS RN 573704-64-0) HPLC YMC | 425.4 [M + H]⁺ |
| 550 | (3S)-1-[3-[2- Fluoro-4- (trifluoromethyl) phenyl]methyl- methyl- amino]azetidine- 1-carbonyl] pyrrolidine-3- carboxamide | 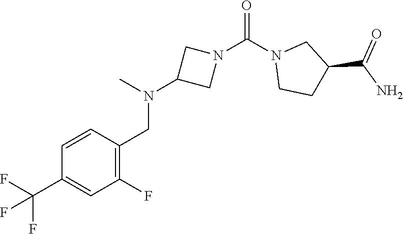 [SMILES: CN(Cc1ccc(C(F)(F)F)cc1F)C1CN(C(=O)N2CC[C@H](C(N)=O)C2)C1] | N-[[2-Fluoro-4- (trifluoromethyl) phenyl]methyl]-N- methyl-azetidin-3- amine;4- methylbenzene- sulfonic acid (BB72) and (3S)- Pyrrolidine-3- carboxamide ( CAS RN 573704-64-0) HPLC YMC | 403.2 [M + H]⁺ |
| 558 | [3-[4-(4-Chloro-2- methylsulfonyl- phenyl)phenyl] azetidin-1-yl]-(3- hydroxyazetidin-1- yl)methanone | 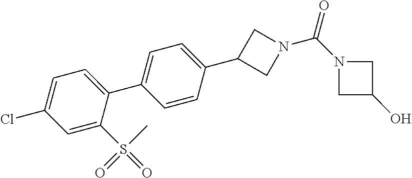 [SMILES: CS(=O)(=O)c1cc(Cl)ccc1-c1ccc(C2CN(C(=O)N3CC(O)C3)C2)cc1] | 3-[4-(4-chloro-2- methylsulfonyl- phenyl)phenyl] azetidine;4- methylbenzene- sulfonic acid (BB44) and Azetidin-3-ol (CAS RN 45347- 82-8) | 421.1 [M + H]⁺ |
| 559 | [3-[4-(4-Chloro-2- methylsulfonyl- phenyl)phenyl] azetidin-1-yl]-(3- hydroxy-3-methyl- azetidin-1- yl)methanone | 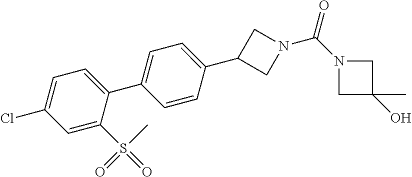 [SMILES: CC1(O)CN(C(=O)N2CC(c3ccc(-c4ccc(Cl)cc4S(C)(=O)=O)cc3)C2)C1] | 3-[4-(4-chloro-2- methylsulfonyl- phenyl)phenyl] azetidine;4- methylbenzene- sulfonic acid (BB44) and 3- Methylazetidin-3- ol; 2,2,2- trifluoroacetic acid (CAS RN 256931- 54-1) HPLC YMC | 435.1 [M + H]⁺ |
| 565 | (3S)-1-[6-[[6- (Trifluoromethyl)- 3-pyridyl]methyl]- 2-azaspiro[3.3] heptane-2- carbonyl] pyrrolidine-3- carboxamide | 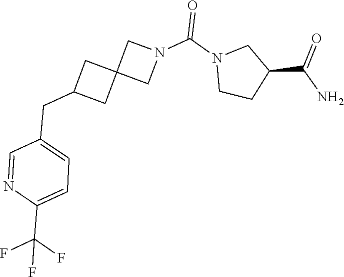 [SMILES: NC(=O)[C@H]1CCN(C(=O)N2CC3(CC(Cc4ccc(C(F)(F)F)nc4)C3)C2)C1] | 6-[[6- (Trifluoromethyl)- 3-pyridyl]methyl]- 2-azaspiro[3.3] heptane; 4- methylbenzene- sulfonic acid (BB73) and (3S)- Pyrrolidine-3- carboxamide ( CAS RN 573704-64-0) HPLC YMC | 397.2 [M + H]⁺ |
| 583 | [6-[[2-Fluoro-4- (trifluoromethyl) phenyl]methyl]-2- azaspiro[3.3] heptan-2-yl]- [(3S)-3- (tetrazol-1- yl)pyrrolidin-1- yl]methanone | 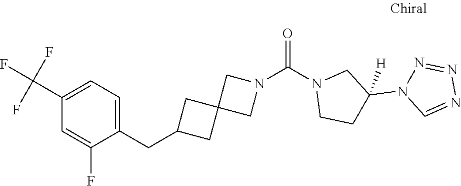 [SMILES: O=C(N1CC[C@H](n2cnnn2)C1)N1CC2(CC(Cc3ccc(C(F)(F)F)cc3F)C2)C1] | 6-[[2-Fluoro-4- (trifluoromethyl) phenyl]methyl]-2- azaspiro[3.3] heptane; 4- methylbenzene- sulfonic acid (BB94) and 1-[(3S)- Pyrrolidin-3- yl]tetrazole (BB86) | 439.2 [M + H]⁺ |
| 610 | [3-[4-(4-Chloro-2- methylsulfonyl- phenyl)phenyl] azetidin-1- yl]-[(3R)-3- (triazol-1- yl)pyrrolidin-1- yl]methanone | 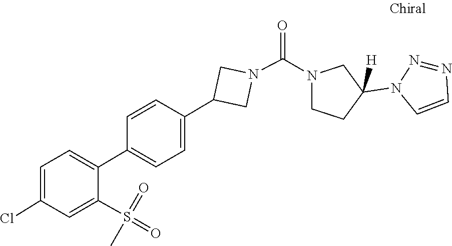 [SMILES: CS(=O)(=O)c1cc(Cl)ccc1-c1ccc(C2CN(C(=O)N3CC[C@@H](n4ccnn4)C3)C2)cc1] | 3-[4-(4-Chloro-2- methylsulfonyl- phenyl)phenyl] azetidine;4- methylbenzene- sulfonic acid (BB44) and 5-[(3R)- Pyrrolidin-3-yl]- 1H-triazole (CAS RN 2166044-57-9) | 486.2 |
| 621 | (3S)-1-[3-[4-[2- Methylsulfonyl-4- (trifluoromethyl) phenyl]phenyl] azetidine-1- carbonyl] pyrrolidine-3- carboxamide | 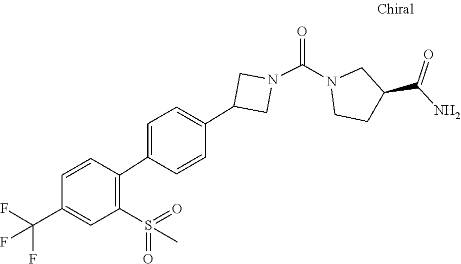 [SMILES: CS(=O)(=O)c1cc(C(F)(F)F)ccc1-c1ccc(C2CN(C(=O)N3CC[C@H](C(N)=O)C3)C2)cc1] | 3-[4-[2- Methylsulfonyl-4- (trifluoromethyl) phenyl]phenyl] azetidine; 4- methylbenzene- sulfonic acid (BB74) and (3S)- Pyrrolidine-3- carboxamide ( CAS RN 573704-64-0) Purification on SiO2 column | 496.2 [M + H]⁺ |
| 623 | [3-[[2-Fluoro-4- (trifluoromethyl) phenyl]methoxy] azetidin-1-yl]- [(3S)-3-(1H- 1,2,4-triazol-5- yl)pyrrolidin-1- yl]methanone | 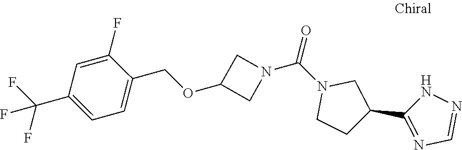 [SMILES: O=C(N1CC(OCc2ccc(C(F)(F)F)cc2F)C1)N1CC[C@H](c2ncn[nH]2)C1] | 3-((2-Fluoro-4- (trifluoromethyl) benzyl)oxy) azetidine;4- methylbenzene- sulfonic acid (CAS RN 2135785-94-1) and 5-[(3S)-Pyrrolidin- 3-yl]-1H-1,2,4- | 414.2 [M + H]⁺ |
| | | | triazole (CAS RN | |
| | | | 2165724-79-6) | |
| | | | HPLC YMC | |
| 628 | (3S)-1-[6-[[5- (Trifluoromethoxy)- 2-pyridyl]methyl]- 2-azaspiro[3.3] heptane-2- carbonyl] pyrrolidine-3- carboxamide | 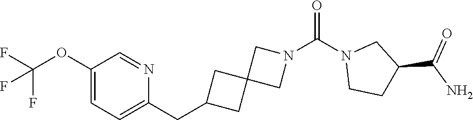 [SMILES: NC(=O)[C@H]1CCN(C(=O)N2CC3(CC(Cc4ccc(OC(F)(F)F)cn4)C3)C2)C1] | 4- Methylbenzene- sulfonic acid; 6-[[5- (trifluoromethoxy)- 2-pyridyl]methyl]- 2-azaspiro[3.3] heptane (CAS 2855958-60-8) and | 413.2 [M + H]⁺ |
| | | | (3S)-Pyrrolidine-3- | |
| | | | carboxamide ( CAS | |
| | | | RN 573704-64-0) | |
| 629 | [(3S)-3-(1H-1,2,4- Triazol-5- yl)pyrrolidin-1-yl]- [6-[[5- (trifluoromethoxy)- 2-pyridyl]methyl]- 2-azaspiro[3.3] heptan-2- yl]methanone | 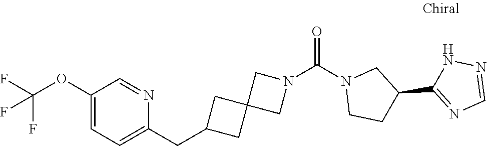 [SMILES: O=C(N1CC[C@H](c2ncn[nH]2)C1)N1CC2(CC(Cc3ccc(OC(F)(F)F)cn3)C2)C1] | 4- Methylbenzene- sulfonic acid; 6-[[5- (trifluoromethoxy)- 2-pyridyl]methyl]- 2-azaspiro[3.3] heptane (CAS 2855958-60-8) and 5-[(3 S)-Pyrrolidin- | 437.2 [M + H]⁺ |
| | | | 3-yl]-1H-1,2,4- | |
| | | | triazole (CAS RN | |
| | | | 2165724-79-6) | |
| | | | HPLC YMC | |
| 650 | [6-[[3-Fluoro-5- (trifluoromethyl)- 2-pyridyl] methyl]-2- azaspiro[3.3] heptan-2-yl]- [(3S)-3-(1H- 1,2,4-triazol-5- yl)pyrrolidin-1- yl]methanone | 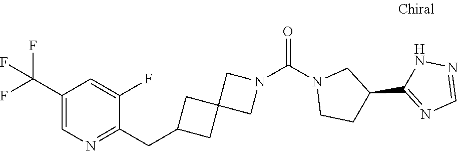 [SMILES: O=C(N1CC[C@H](c2ncn[nH]2)C1)N1CC2(CC(Cc3ncc(C(F)(F)F)cc3F)C2)C1] | 6-[[3-Fluoro-5- (trifluoromethyl)-2- pyridyl]methyl]-2- azaspiro[3.3] heptane;4. methylbenzene- sulfonic acid (BB106) and 5-[(3S)- Pyrrolidin-3-yl]- | 439.3 [M + H]⁺ |
| | | | 1H-1,2,4-triazole | |
| | | | (CAS RN 2165724- | |
| | | | 79-6) | |
| | | | HPLC YMC | |
| 653 | [6-[[4-Fluoro-2- (trifluoromethyl) phenyl]methyl]- 2-azaspiro[3.3] heptan-2-yl]- [(3S)-3-(1H- 1,2,4-triazol-5- yl)pyrrolidin-1- yl]methanone | 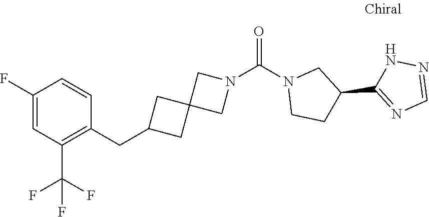 [SMILES: O=C(N1CC[C@H](c2ncn[nH]2)C1)N1CC2(CC(Cc3ccc(F)cc3C(F)(F)F)C2)C1] | 6-[[4-Fluoro-2- (trifluoromethyl) phenyl]methyl]-2- azaspiro[3.3] heptane;4- methylbenzene- sulfonic acid (BB107) and 5-[(3S)- Pyrrolidin-3-yl]- 1H-1,2,4-triazole (CAS RN 2165724- 79-6) HPLC YMC | 438.3 [M + H]⁺ |
| 654 | [6-[[2-Fluoro-4- (trifluoromethyl) phenyl]methyl]- 2-azaspiro[3.3] heptan-2-yl]- [(3S)-3-(1H- 1,2,4-triazol-5- yl)pyrrolidin-1- yl]methanone | 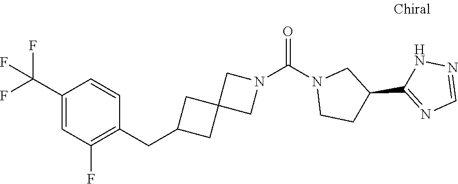 [SMILES: O=C(N1CC[C@H](c2ncn[nH]2)C1)N1CC2(CC(Cc3ccc(C(F)(F)F)cc3F)C2)C1] | 6-[[2-Fluoro-4- (trifluoromethyl) phenyl]methyl]-2- azaspiro[3.3] heptane; 4- methylbenzene- sulfonic acid (BB94) and 5-[(3S)- Pyrrolidin-3-yl]- 1H-1,2,4-triazole (CAS RN 2165724- | 438.3 [M + H]⁺ |
| | | | 79-6) | |
| | | | HPLC YMC | |
| 670 | [3-[4-[3- (Difluoromethyl)- 5-methyl-pyrazol- 1-yl]phenyl] azetidin- 1-yl]-[(3S)-3-(1H- 1,2,4-triazol-5- yl)pyrrolidin-1- yl]methanone | 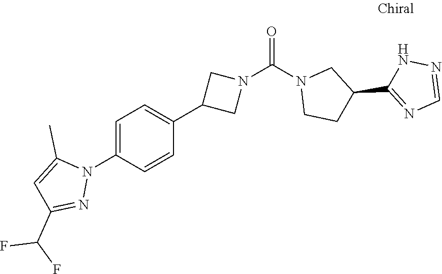 [SMILES: Cc1cc(C(F)F)nn1-c1ccc(C2CN(C(=O)N3CC[C@H](c4ncn[nH]4)C3)C2)cc1] | 1-[4-(Azetidin-3- yl)phenyl]-3- (difluoromethyl)-5- methyl-pyrazole;4- methylbenzene- sulfonic acid (BB75) and 5-[(3S)- Pyrrolidin-3-yl]- 1H-1,2,4-triazole (CAS RN 2165724- 79-6) HPLC YMC | 428.3 [M + H]⁺ |
| 671 | [3-[4-[5-Methyl-3- (trifluoromethyl) pyrazol-1- yl]phenyl]azetidin- 1-yl]-[(3S)-3-(1H- 1,2,4-triazol-5- yl)pyrrolidin-1- yl]methanone | 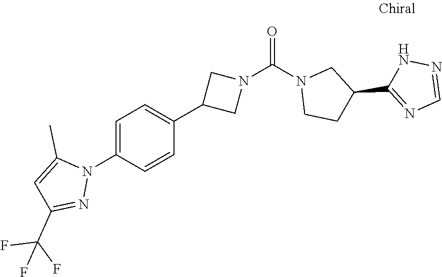 [SMILES: Cc1cc(C(F)(F)F)nn1-c1ccc(C2CN(C(=O)N3CC[C@H](c4ncn[nH]4)C3)C2)cc1] | 1-[4-(Azetidin-3- yl)phenyl]-5- methyl-3- (trifluoromethyl) pyrazole;4- methylbenzene- sulfonic acid (BB76) and 5-[(3S)- Pyrrolidin-3-yl]- 1H-1,2,4-triazole (CAS RN 2165724- 79-6) HPLC YMC | 446.3 [M + H]⁺ |
| 673 \| | (3S)-1-[6-[[3- Fluoro-5- (trifluoromethyl)-2- pyridyl]methyl]-2- azaspiro[3.3] heptane-2- carbonyl] pyrrolidine-3- carboxamide | 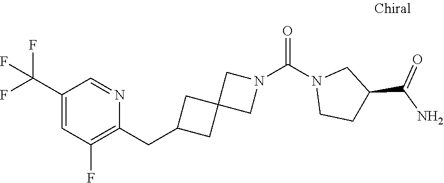 [SMILES: NC(=O)[C@H]1CCN(C(=O)N2CC3(CC(Cc4ncc(C(F)(F)F)cc4F)C3)C2)C1] | 6-[[3-Fluoro-5- (trifluoromethyl)-2- pyridyl]methyl]-2- azaspiro[3.3] heptane;4- methylbenzene- sulfonic acid (BB106) and (3S)- Pyrrolidine-3- carboxamide ( CAS RN 573704-64-0) HPLC YMC | 415.3 [M + H]⁺ |
| 679 | [rac-(3S)-3-(1H- 1,2,4-Triazol-5- yl)pyrrolidin-1-yl]- [6-[3- (trifluoromethyl- sulfonimidoyl) phenyl]methyl]-2- azaspiro[3.3] heptan-2- yl]methanone | 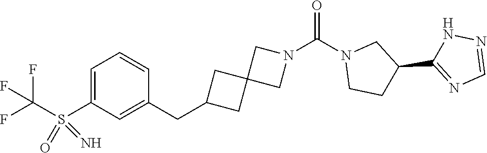 [SMILES: N=S(=O)(c1cccc(CC2CC3(C2)CN(C(=O)N2CC[C@H](c4ncn[nH]4)C2)C3)c1)C(F)(F)F] | [3-(2- Azaspiro[3.3] heptan-6- ylmethyl)phenyl]- imino-oxo- (trifluoromethyl)- 26-sulfane; 4- methylbenzene- sulfonic acid (BB108) and 5-[(3S)- | 483.4 [M + H]⁺ |
| | | | Pyrrolidin-3-yl]- | |
| | | | 1H-1,2,4-triazole | |
| | | | (CAS RN 2165724- | |
| | | | 79-6) | |
| | | | HPLC YMC | |
| 689 | [3-[4-[3-(2,2- Dimethylpropyl) triazol-4- yl]phenyl]azetidin- 1-yl]-[(3S)-3-(1H- 1,2,4-triazol-5- yl)pyrrolidin-1- yl]methanone | 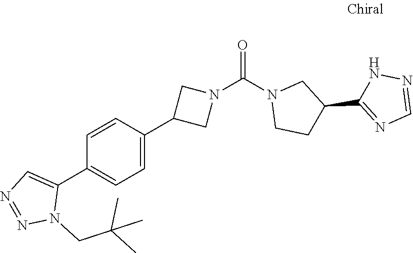 [SMILES: CC(C)(C)Cn1nncc1-c1ccc(C2CN(C(=O)N3CC[C@H](c4ncn[nH]4)C3)C2)cc1] | 5-[4-(Azetidin-3- yl)phenyl]-1-(2,2- dimethylpropyl) triazole 4- methylbenzene- sulfonic acid (BB45) and 5-[(3S)- Pyrrolidin-3-yl]- 1H-1,2,4-triazole (CAS RN 2165724- 79-6) HPLC YMC | 435.5 [M + H]⁺ |
| 693 | [(3S)-3-(1H-1,2,4- Triazol-5- yl)pyrrolidin-1-yl]- [3-[3-[5- (trifluoromethyl) pyrazin-2-yl]oxy- 1-bicyclo[1.1.1] pentanyl]azetidin- 1-yl]methanone | 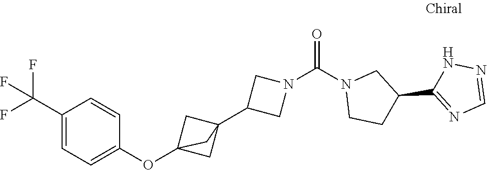 [SMILES: O=C(N1CC(C23CC(Oc4ccc(C(F)(F)F)cc4)(C2)C3)C1)N1CC[C@H](c2ncn[nH]2)C1] | 2-[[3-(Azetidin-3- yl)-1-bicyclo [1.1.1]pentanyl] oxy]-5- (trifluoromethyl) pyrazine;4- methylbenzene- sulfonic acid (BB109) and 5-[(3S)- Pyrrolidin-3-yl]- 1H-1,2,4-triazole | 450.4 [M + H]⁺ |
| | | | (CAS RN 2165724- | |
| | | | 79-6) | |
| | | | HPLC YMC | |
| 695 | [3-[4-(3,5- Diisopropyl- pyrazol-1- yl)phenyl]azetidin- 1-yl]-[(3S)-3-(1H- 1,2,4-triazol-5- yl)pyrrolidin-1- yl]methanone | 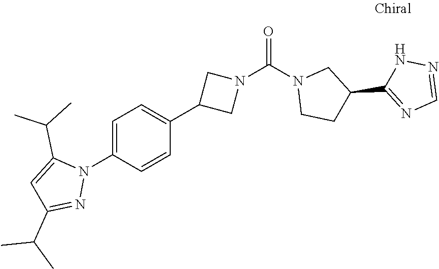 [SMILES: CC(C)c1cc(C(C)C)n(-c2ccc(C3CN(C(=O)N4CC[C@H](c5ncn[nH]5)C4)C3)cc2)n1] | 1-[4-(Azetidin-3- yl)phenyl]-3,5- diisopropyl- pyrazole;4- methylbenzene- sulfonic acid (BB77) and 5-[(3S)- Pyrrolidin-3-yl]- 1H-1,2,4-triazole (CAS RN 2165724-79-6) HPLC YMC | 446.5 [M + H]⁺ |
| 697 | [(3S)-3-(4H-1,2,4- Triazol-3- yl)pyrrolidin-1-yl]- [7-[5- (trifluoromethyl)- 2-pyridyl] amino]-2- azaspiro[3.5] nonan- 2-yl]methanone | 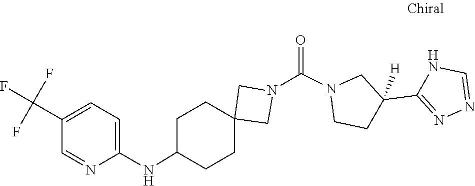 [SMILES: O=C(N1CC[C@H](c2nnc[nH]2)C1)N1CC2(CCC(Nc3ccc(C(F)(F)F)cn3)CC2)C1] | N-[6- (Trifluoromethyl)- 3-pyridyl]-2- azaspiro[3.5]nonan- 7-amine; 4- methylbenzene- sulfonic acid (BB95) and 3-[(3S)- Pyrrolidin-3-yl]- 4H-1,2,4-triazole (CAS RN 2165724- 79-6) | 450.3 [M + H]⁺ |
| 700 | [3-[[2-Fluoro-4- (trifluoromethyl- sulfonyl)phenyl] methoxy]azetidin- 1-yl]-[(3S)-3- (4H-1,2,4- triazol-3- yl)pyrrolidin-1- yl]methanone | 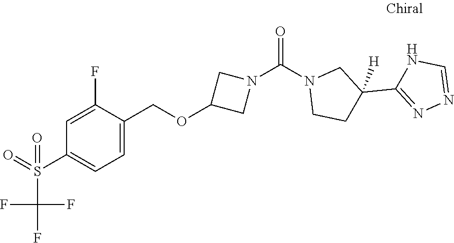 [SMILES: O=C(N1CC(OCc2ccc(S(=O)(=O)C(F)(F)F)cc2F)C1)N1CC[C@H](c2nnc[nH]2)C1] | 3-[[2-Fluoro-4- (trifluoromethyl- sulfonyl)phenyl] methoxy]azetidine; 4-methylbenzene- sulfonic acid (BB96) and 3-[(3S)- Pyrrolidin-3-yl]- 4H-1,2,4-triazole (CAS RN 2165724-79-6) | 478.2 [M + H]⁺ |
| 701 | [(3S)-3-(4H-1,2,4- Triazol-3- yl)pyrrolidin-1-yl]- [7-[[6- (trifluoromethyl)- 3-pyridyl]methyl]- 2-azaspiro [3.5]nonan- 2-yl]methanone | 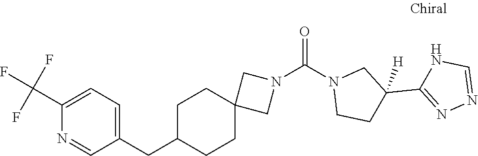 [SMILES: O=C(N1CC[C@H](c2nnc[nH]2)C1)N1CC2(CCC(Cc3ccc(C(F)(F)F)nc3)CC2)C1] | 7-[6- (Trifluoromethyl)- 3-pyridyl]methyl]- 2-azaspiro[3.5] nonane; 4- methylbenzene- sulfonic acid (BB97) and 3-[(3S)- Pyrrolidin-3-yl]- 4H-1,2,4-triazole | 449.3 [M + H]⁺ |
| | | | (CAS RN 2165724- | |
| | | | 79-6) | |
| 715 | [3-[4-[1-Methyl-3- (trifluoromethyl) pyrazol-4- yl]phenyl]azetidin- 1-yl]-[(3S)-3-(4H- 1,2,4-triazol-3- yl)pyrrolidin-1- yl]methanone | 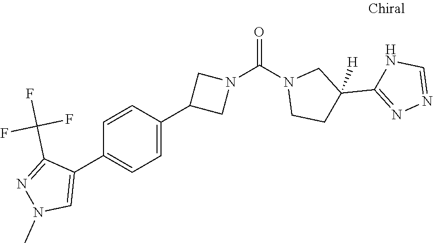 [SMILES: Cn1cc(-c2ccc(C3CN(C(=O)N4CC[C@H](c5nnc[nH]5)C4)C3)cc2)c(C(F)(F)F)n1] | 4-[4-(Azetidin-3- yl)phenyl]-1- methyl-3- (trifluoromethyl) pyrazole;4- methylbenzene- sulfonic acid (BB98) and 5-[(3S)- Pyrrolidin-3-yl]- 1H-1,2,4-triazole (CAS RN 2165724- 79-6) | 446.3 [M + H]⁺ |
| 734 | 4-[[2-[(3S)-3-(1H- 1,2,4-Triazol-5- yl)pyrrolidine-1- carbonyl]-2- azaspiro[3.3] heptan-6- yl]methyl]-2- (trifluoromethyl) benzonitrile | 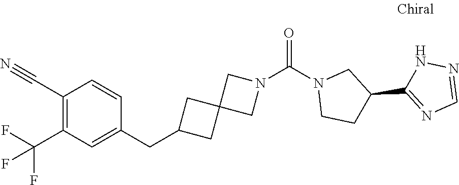 [SMILES: N#Cc1ccc(CC2CC3(C2)CN(C(=O)N2CC[C@H](c4ncn[nH]4)C2)C3)cc1C(F)(F)F] | 4-(2- Azaspiro[3.3] heptan- 6-ylmethyl)-2- (trifluoromethyl) benzonitrile;4- methylbenzene- sulfonic acid (CAS RN 2855958- 25-5) and 5-[(3S)- Pyrrolidin-3-yl]- 1H-1,2,4-triazole | 445.3 [M + H]⁺ |
| | | | (CAS RN 2165724- | |
| | | | 79-6) | |
| | | | HPLC YMC | |
| 735 | [(3S)-3-(1H-1,2,4- Triazol-5- yl)pyrrolidin-1-yl]- [6-[[6- (trifluoromethoxy)- 3-pyridyl]methyl]- 2-azaspiro[3.3] heptan-2- yl]methanone | 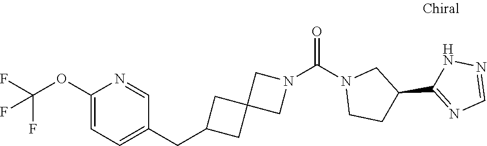 [SMILES: O=C(N1CC[C@H](c2ncn[nH]2)C1)N1CC2(CC(Cc3ccc(OC(F)(F)F)nc3)C2)C1] | 4-Methylbenzene- sulfonic acid; 6-[[6- (trifluoromethoxy)- 3-pyridyl]methyl]- 2-azaspiro[3.3] heptane (CAS RN 2855958- 60-8) and 5-[(3S)- Pyrrolidin-3-yl]- | 437.3 [M + H]⁺ |
| | | | 1H-1,2,4-triazole | |
| | | | (CAS RN 2165724- | |
| | | | 79-6) | |
| | | | HPLC YMC | |
| 736 | [(3S)-3-(1H-1,2,4- Triazol-5- yl)pyrrolidin-1-yl]- [6-[[3- (trifluoromethyl- sulfonyl)phenyl] methyl]-2- azaspiro[3.3] heptan-2- yl]methanone | 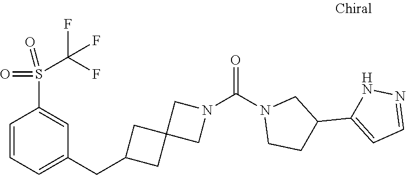 [SMILES: O=C(N1CCC(c2ccn[nH]2)C1)N1CC2(CC(Cc3cccc(S(=O)(=O)C(F)(F)F)c3)C2)C1] | 4-Methylbenzene- sulfonic acid; 6-[[3- (trifluoromethyl- sulfonyl)phenyl] methyl]-2- azaspiro[3.3] heptane (CAS RN 2763646-33-7) and 5-[(3S)-Pyrrolidin- 3-yl]-1H-1,2,4- triazole (CAS RN | 484.3 [M + H]⁺ |
| | | | 2165724-79-6) | |
| | | | HPLC YMC | |
| 754 | (3S)-1-[3-[4-[5- Methyl-3- (trifluoromethyl) pyrazol-1- yl]phenyl]azetidine- 1-carbonyl] pyrrolidine-3- carboxamide | 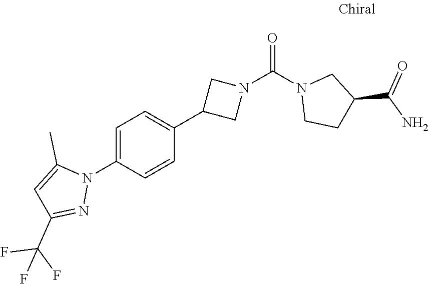 [SMILES: Cc1cc(C(F)(F)F)nn1-c1ccc(C2CN(C(=O)N3CC[C@H](C(N)=O)C3)C2)cc1] | 1-[4-(Azetidin-3- yl)phenyl]-5- methyl-3- (trifluoromethyl) pyrazole;4- methylbenzene- sulfonic acid (BB76) and (3S)- Pyrrolidine-3- carboxamide (CAS RN 573704-64-0) HPLC YMC | 422.2 [M + H]⁺ |
| 761 | [4-[[2-Fluoro-4- (trifluoromethoxy) phenyl]methoxy]-1- piperidyl]-[(3S)-3- (1H-1,2,4-triazol-5- yl)pyrrolidin-1- yl]methanone | 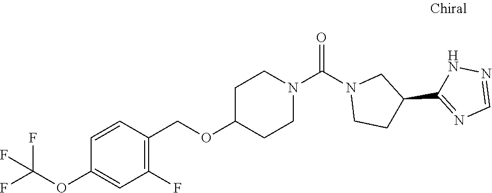 [SMILES: O=C(N1CCC(OCc2ccc(OC(F)(F)F)cc2F)CC1)N1CC[C@H](c2ncn[nH]2)C1] | 4-[[2-Fluoro-4- (trifluoromethoxy) phenyl]methoxy] piperidine (BB 85) and 5-[(3S)- Pyrrolidin- 3-yl]-1H-1,2,4- triazole (CAS RN 2165724-79-6) HPLC YMC | 458.2 [M + H]⁺ |
| 765 | Methyl 5-[[2-[(3S)- 3-(1H-1,2,4-triazol- 5-yl)pyrrolidine-1- carbonyl]-2- azaspiro[3.3] heptan-6- yl]methyl]-2- (trifluoromethoxy) benzoate | 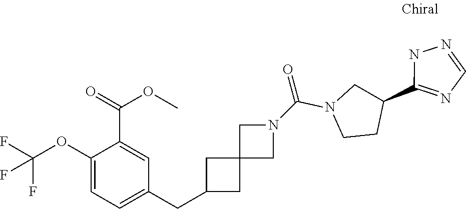 [SMILES: COC(=O)c1cc(CC2CC3(C2)CN(C(=O)N2CC[C@H](c4ncn[nH]4)C2)C3)ccc1OC(F)(F)F] | 5-(2- Azaspiro[3.3] heptan- 6-ylmethyl)-2- (trifluoromethoxy) benzoic acid methyl ester;4- methylbenzene- sulfonic acid (BB92) and 5-[(3S)- pyrrolidin-3-yl]- 1H-1,2,4-triazole (CAS RN 2165724- 79-6) | 494.2 [M + H]⁺ |
| | | | HPLC YMC | |
| 768 | [(3S)-3-(1H-1,2,4- Triazol-5- yl)pyrrolidin-1-yl]- [6-[[5- (trifluoromethyl- sulfonyl)-2- pyridyl]methyl]-2- azaspiro[3.3] heptan-2- yl]methanone | 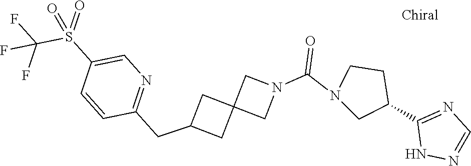 [SMILES: O=C(N1CC[C@H](c2ncn[nH]2)C1)N1CC2(CC(Cc3ccc(S(=O)(=O)C(F)(F)F)cn3)C2)C1] | 4-Methylbenzene- sulfonic acid; 6-[[5- (trifluoromethyl- sulfonyl)-2- pyridyl]methyl]-2- azaspiro[3.3] heptane (BB117) and 5-[(3S)- Pyrrolidin-3-yl]- 1H-1,2,4-triazole | 485.2 [M + H]⁺ |
| | | | (CAS RN 2165724- | |
| | | | 79-6) | |
| 771 | [(3S)-3-(1H-1,2,4- Triazol-5- yl)pyrrolidin- 1-yl]-[6-[[5-[3- (trifluoromethyl) azetidin-1- yl]pyrazin- 2-yl]methyl]-2- azaspiro[3.3] heptan-2- yl]methanone | 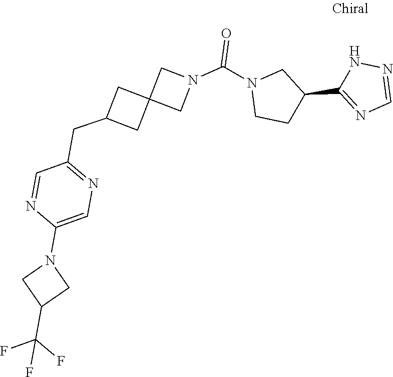 [SMILES: O=C(N1CC[C@H](c2ncn[nH]2)C1)N1CC2(CC(Cc3cnc(N4CC(C(F)(F)F)C4)cn3)C2)C1] | 2,2,2- Trifluoroacetic acid; 6-[5-[3- (trifluoromethyl) azetidin-1- yl]pyrazin- 2-yl]methyl]-2- azaspiro[3.3] heptane (BB118) and 5-[(3S)- Pyrrolidin-3-yl]- 1H-1,2,4-triazole (CAS RN 2165724-79-6) | 475.3 [M − H]⁻ |
| 772 | [(3S)-3-(4H-1,2,4- Triazol-3- yl)pyrrolidin-1-yl]- [6-[[5-[[1- (trifluoromethyl) cyclopropyl] methylamino] pyrazin-2- yl]methyl]-2- azaspiro [3.3]heptan- 2-yl]methanone | 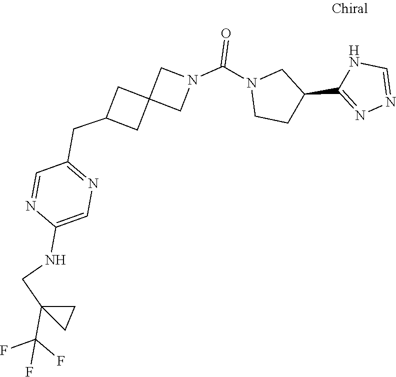 [SMILES: O=C(N1CC[C@H](c2nnc[nH]2)C1)N1CC2(CC(Cc3cnc(NCC4(C(F)(F)F)CC4)cn3)C2)C1] | 5-(2- Azaspiro[3.3] heptan-6- ylmethyl)- N-[[1- (trifluoromethyl) cyclopropyl] methyl] pyrazin-2- amine;2,2,2- trifluoroacetic acid (BB119) and 5- [(3S)-Pyrrolidin-3- yl]-1H-1,2,4- triazole (CAS RN 2165724-79-6) | 489.4 [M − H]⁻ |
| 776 | [(3S)-3-(1H-1,2,4- Triazol-5- yl)pyrrolidin-1-yl]- [(6S or 6R)-6-[[3- (trifluoromethyl- sulfonyl)phenyl] methyl1-2- azaspiro[3.4]octan- 2-yl]methanone | 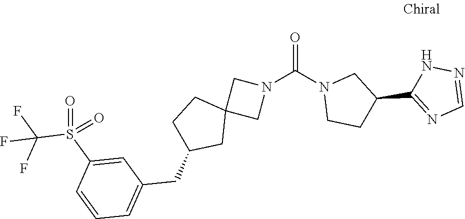 [SMILES: O=C(N1CC[C@H](c2ncn[nH]2)C1)N1CC2(CC[C@@H](Cc3cccc(S(=O)(=O)C(F)(F)F)c3)C2)C1] | 4-Methylbenzene- sulfonic acid;(6S or 6R)-6-[[3- (trifluoromethyl- sulfonyl)phenyl] methyl]-2- azaspiro[3.4]octane (BB110) and 5- [(3S)-Pyrrolidin-3- yl]-1H-1,2,4- triazole (CAS RN 2165724-79-6) HPLC YMC | 498.3 [M + H]⁺ |
| 780 | [6-[1-Methyl-5- (trifluoromethyl) pyrazol-4-yl] methyl]-2- azaspiro[3.3]heptan- 2-yl]-[(3S)-3-(1H- 1,2,4-triazol-5- yl)pyrrolidin-1- yl]methanone | 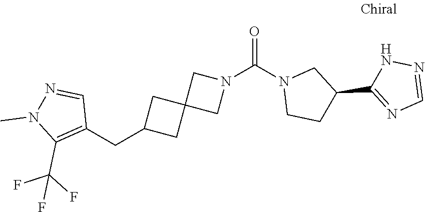 [SMILES: Cn1ncc(CC2CC3(C2)CN(C(=O)N2CC[C@H](c4ncn[nH]4)C2)C3)c1C(F)(F)F] | 4-Methylbenzene- sulfonic acid;(6S)- 6-[[3- (trifluoromethyl- sulfonyl)phenyl] methyl 1-2- azaspiro[3.4]octane (BB111) and 5- [(3S)-Pyrrolidin-3- yl]-1H-1,2,4- triazole (CAS RN 2165724-79-6) | 424.3 [M + H]⁺ |
| 790 | [(3S)-3-(1H-1,2,4- Triazol-5- yl)pyrrolidin-1-yl]- [3-[3-[[5- (trifluoromethyl) pyrazin-2- yl]amino]-1- bicyclo[1.1.1] pentanyl]azetidin- 1-yl]methanone | 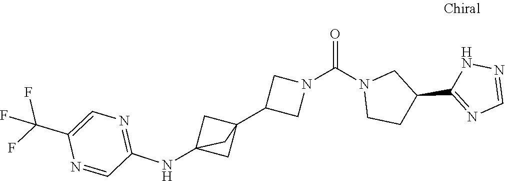 [SMILES: O=C(N1CC(C23CC(Nc4cnc(C(F)(F)F)cn4)(C2)C3)C1)N1CC[C@H](c2ncn[nH]2)C1] | N-[3-(Azetidin-3- yl)-1-bicyclo[1.1.1] pentanyl]-5- (trifluoromethyl) pyrazin-2- amine;2,2,2- trifluoroacetic acid (BB116) and 5- [(3S)-P yrrolidin-3-yl]-1H- 1,2,4-triazole (CAS RN 2165724-79-6) | 449.2 [M + H]⁺ |
| 804 | (3S)-1-[3-[3-(4- Chloro-2- methylsulfonyl- phenyl)-1- bicyclo[1.1.1] pentanyl]azetidine- 1-carbonyl] pyrrolidine-3- carboxamide | 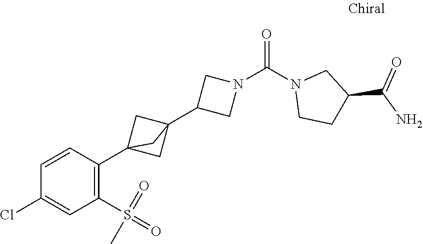 [SMILES: CS(=O)(=O)c1cc(Cl)ccc1C12CC(C3CN(C(=O)N4CC[C@H](C(N)=O)C4)C3)(C1)C2] | 3-[3-(4-Chloro-2- methylsulfonyl- phenyl)-1- bicyclo[1.1.1] pentanyl]azetidine; 4-methylbenzene- sulfonic acid (BB78) and (3S)- Pyrrolidine-3- carboxamide ( CAS RN 573704-64-0) Purification on SiO2 column | 452.1 [M + H]⁺ |
| 808 | (3S)-1-[3-[3-[2- Methylsulfonyl-4- (trifluoromethyl) phenyl]-1- bicyclo[1.1.1] pentanyl]azetidine- 1-carbonyl] pyrrolidine-3- carboxamide | 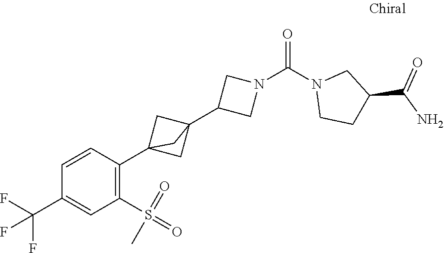 [SMILES: CS(=O)(=O)c1cc(C(F)(F)F)ccc1C12CC(C3CN(C(=O)N4CC[C@H](C(N)=O)C4)C3)(C1)C2] | 3-[3-[2- Methylsulfonyl-4- (trifluoromethyl) phenyl]-1- bicyclo[1.1.1] pentanyl]azetidine; 4-methylbenzene- sulfonic acid (BB79) and (3S)- Pyrrolidine-3- carboxamide ( CAS RN 573704-64-0) Purification on SiO2 column | 486.1 [M + H]⁺ |
| 813 | [3-[3-(4- Fluorophenyl)-1- bicyclo[1.1.1] pentanyl]azetidin- 1-yl]-[(3S)-3- (1H-1,2,4-triazol- 5-yl)pyrrolidin-1- yl]methanone | 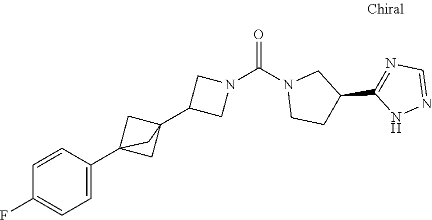 [SMILES: O=C(N1CC(C23CC(c4ccc(F)cc4)(C2)C3)C1)N1CC[C@H](c2ncn[nH]2)C1] | 3-[3-(4- Fluorophenyl)-1- bicyclo[1.1.1] pentanyl]azetidine; 2,2,2- trifluoroacetic acid (BB112) and 5- [(3S)-Pyrrolidin-3- yl]-1H-1,2,4- triazole (CAS RN 2165724-79-6) HPLC YMC | 382.2 [M + H]⁺ |
| 822 | [(3S)-3-(1H-1,2,4- Triazol-5- yl)pyrrolidin-1-yl]- [6-[[4- (trifluoromethyl- sulfonyl)phenyl] methyl 1-2- azaspiro[3.3]heptan- 2-yl]methanone | 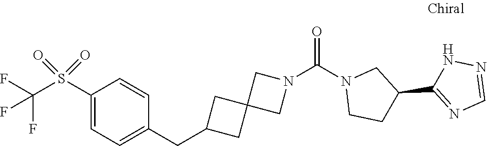 [SMILES: O=C(N1CC[C@H](c2ncn[nH]2)C1)N1CC2(CC(Cc3ccc(S(=O)(=O)C(F)(F)F)cc3)C2)C1] | 2,2,2- Trifluoroacetic acid; 6-[[4- (trifluoromethyl- sulfonyl)phenyl] methyl1-2- azaspiro[3.3] heptane (BB113) and 5- | 484.2 [M + H]⁺ |
| | | | [(3S)-Pyrrolidin-3- | |
| | | | yl]-1H-1,2,4- | |
| | | | triazole (CAS RN | |
| | | | 2165724-79-6) | |
| | | | HPLC YMC | |
| 831 | [(3S)-3-(1H-1,2,4- Triazol-5- yl)pyrrolidin-1-yl]- [3-[3-[3- (trifluoromethoxy) phenyl]-1- bicyclo[1.1.1] pentanyl]azetidin- 1-yl]methanone | 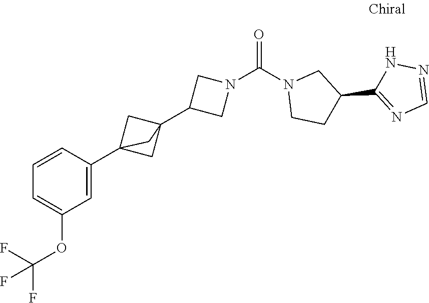 [SMILES: O=C(N1CC(C23CC(c4cccc(OC(F)(F)F)c4)(C2)C3)C1)N1CC[C@H](c2ncn[nH]2)C1] | 2,2,2- Trifluoroacetic acid; 3-[3-[3- (trifluoromethoxy) phenyl]-1- bicyclo[1.1.1] pentanyl]azetidine (BB114) and 5- [(3S)-Pyrrolidin-3- yl]-1H-1,2,4- triazole (CAS RN 2165724-79-6) | 448.3 [M + H]⁺ |
| 840 | [(3S)-3-(1H-1,2,4- Triazol-5- yl)pyrrolidin-1-yl]- [3-[3-[4- (trifluoromethyl- sulfonyl)phenyl]-1- bicyclo[1.1.1] pentanyl]azetidin- 1-yl]methanone | 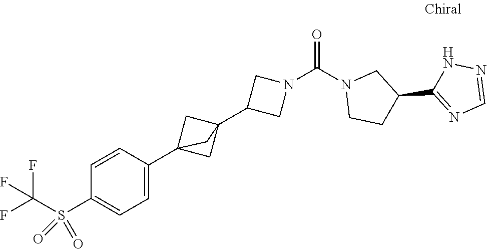 [SMILES: O=C(N1CC(C23CC(c4ccc(S(=O)(=O)C(F)(F)F)cc4)(C2)C3)C1)N1CC[C@H](c2ncn[nH]2)C1] | 2,2,2- Trifluoroacetic acid;3-[3-[4- (trifluoromethyl- sulfonyl)phenyl]- 1-bicyclo[1.1.1] pentanyl]azetidine (BB115) and 5- [(3S)-Pyrrolidin-3- yl]-1H-1,2,4- triazole (CAS RN 2165724-79-6) HPLC YMC | 496.2 [M + H]⁺ |
| 841 | [3-[3-(4-Chloro-2- methylsulfonyl- phenyl)-1- bicyclo[1.1.1] pentanyl]azetidin- 1-yl]-[(3S)-3- (1H-1,2,4- triazol-5- yl)pyrrolidin-1- yl]methanone | 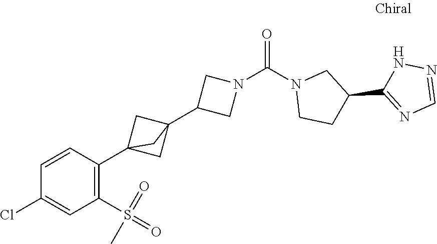 [SMILES: CS(=O)(=O)c1cc(Cl)ccc1C12CC(C3CN(C(=O)N4CC[C@H](c5ncn[nH]5)C4)C3)(C1)C2] | 3-[3-(4-Chloro-2- methylsulfonyl- phenyl)-1- bicyclo[1.1.1] pentanyl]azetidine; 4-methylbenzene- sulfonic acid (BB78) and 5 -[(3S)- Pyrrolidin-3-yl]- 1H-1,2,4-triazole (CAS RN 2165724-79-6) | 476.1 [M + H]⁺ |

### Example 8

### [4-[(2-Chloro-4-fluoro-phenyl)methoxy]-1-piperidyl]-[3-(4-methyl-1H-pyrazol-5-yl)azetidin-1-yl]methanone

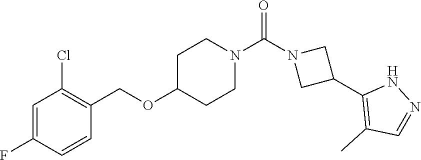 [SMILES: Cc1cn[nH]c1C1CN(C(=O)N2CCC(OCc3ccc(F)cc3Cl)CC2)C1]

To a solution of 5-(azetidin-3-yl)-4-methyl-1H-pyrazole 2,2,2-trifluoroacetic acid (20 mg, 80 μmol, CAS RN 1361115-19-6) in DCM (1 mL) was added DIPEA (42 μL, 239 μmol) followed by addition of 4-nitrophenyl carbonochloridate (15.2 mg, 75.6 μmol, CAS RN 7693-46-1) in one portion. The mixture was stirred at RT for 2 hours. The reaction mixture was evaporated to dryness. The residue was taken up in dioxane (1 mL). After addition of 4-[(2-Chloro-4-fluorophenyl)methoxy]piperidine (28 mg, 80 μmol, CAS RN 1098356-25-2) in one portion, the mixture was stirred at RT for 2 hours. An aqueous 2 M NaOH (796 μl, 1.59 mmol) solution was added and the RM heated to 80° C. overnight. After cooling down the mixture was extracted three times with EtOAc. The organic layers were dried over MgSO₄, filtered and evaporated. The product was purified using HPLC Method YMC to get the desired compound as a yellow amorphous (0.002 g; 6.72%). MS (ESI): m/z=407.2 [M+H]⁺.

### Example 13

### [3-[[2-Fluoro-4-(trifluoromethyl)phenyl]methoxy]azetidin-1-yl]-[3-(2-methylpyrazol-3-yl)pyrrolidin-1-yl]methanone

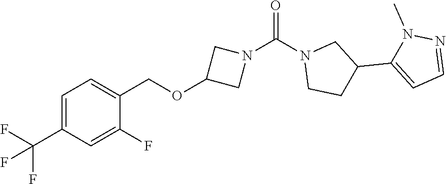 [SMILES: Cn1nccc1C1CCN(C(=O)N2CC(OCc3ccc(C(F)(F)F)cc3F)C2)C1]

To an ice-cold solution of (3-(1H-pyrazol-5-yl)pyrrolidin-1-yl)(3-((2-fluoro-4-(trifluoromethyl)benzyl)oxy)azetidin-1-yl)methanone (73 mg, 177 μmol, example 1) in THF (1 mL), potassium tert-butoxide (23.8 mg, 212 μmol) was added. To the RM, iodomethane (12.2 μl, 195 μmol) was added dropwise and stirred at OC for 5 min. RM was warmed to RT and continued stirring for 30 min. The RM was quenched with water and poured over EtOAc. The aqeuous layer was extracted by EtOAc three times and combined organic layers dried over MgSO₄. Solvent was removed in vacuo. Crude product was purified by HPLC Method GN. Two samples were collected of the same mass. It was assumed this was due to methylation to desired product and tautomer. 2D NMR confirmed first eluting compound was desired product, obtained as a colorless oil (10 mg; 12.6%). MS (ESI): m/z=427.3 [M+H]⁺.

### Example 17

### [3-[4-(2,4-Dichlorophenyl)phenyl]azetidin-1-yl]-[3-(1H-pyrazol-5-yl)pyrrolidin-1-yl]methanone

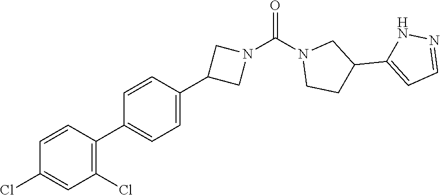 [SMILES: O=C(N1CC(c2ccc(-c3ccc(Cl)cc3Cl)cc2)C1)N1CCC(c2ccn[nH]2)C1]

A suspension of (2,4-dichlorophenyl) boronic acid (104 mg, 547 μmol, CAS RN 68716-47-2), (3-(1H-pyrazol-5-yl)pyrrolidin-1-yl)(3-(4-bromophenyl)azetidin-1-yl)methanone (205.2 mg, 547 μmol, BB3), potassium carbonate (378 mg, 2.73 mmol), Water (257 μL) and tetrakis(triphenylphosphine)palladium(0)(31.6 mg, 27.3 μmol) in THF (2.57 mL) was stirred at 80° C. for 3 hours. The reaction mixture was poured on water and EtOAc and the layers were separated. The aqueous layer was extracted twice with EtOAc. The organic layers were dried over MgSO₄, filtered, treated with silica gel and evaporated. The compound was purified by silica gel chromatography on a 25 g column using an MPLC system eluting with a gradient of n-heptane:EtOAc/ethanol (3/1)(70:30 to 10:90) to get the desired compound as a colorless solid. The product was purified using HPLC Method YMC affording desired product as a colourless foam (47.3 mg; 19.6%). MS (ESI): m/z=441.2 [M+H]⁺.

### Example 42

### (+)- or (−)-[2-(2-Chloro-4-fluoro-phenoxy)-7-azaspiro[3.5]nonan-7-yl]-[(3R)-3-(1H-1,2,4-triazol-5-yl)pyrrolidin-1-yl]methanone and Example 43 (−)- or (+)-[2-(2-Chloro-4-fluoro-phenoxy)-7-azaspiro[3.5]nonan-7-yl]-[(3S)-3-(1H-1,2,4-triazol-5-yl)pyrrolidin-1-yl]methanone

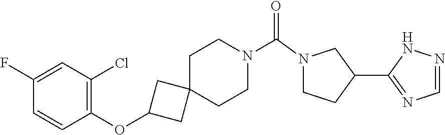 [SMILES: O=C(N1CCC2(CC1)CC(Oc1ccc(F)cc1Cl)C2)N1CCC(c2ncn[nH]2)C1]

To a solution of 2-(2-chloro-4-fluorophenoxy)-7-azaspiro[3.5]nonane (38 mg, 143 μmol, CAS RN 2135785-78-1) in ACN (1.5 mL) was added TEA (160 μL, 1.5 mmol) followed by addition of di(1H-1,2,4-triazol-1-yl)methanone (23.5 mg, 143 μmol) in one portion. The mixture was stirred at RT for 30 minutes. After addition of 5-(pyrrolidin-3-yl)-1H-1,2,4-triazole dihydrochloride (30.3 mg, 143 μmol, CAS RN 1909337-56-9) in one portion, the clear solution was stirred at 65° C. overnight. The reaction mixture was poured on water and EtOAc and the layers were separated. The aqueous layer was extracted with EtOAc. The organic layers were washed with aqueous 5% NaHCO₃ solution, aqueous 0.5 M HCl solution and brine, dried over MgSO₄, filtered and evaporated. The racemate was separated by SFC Method IC1 to get the desired compound 42 (first eluting) as a light yellow lyoph solid (0.022 g; 31.8%). MS (ESI): m/z=434.3 [M+H]⁺ and compound 43 (second eluting) as a light yellow lyoph solid (0.025 g; 36.2%). MS (ESI): m/z=434.3 [M+H]⁺.

If not indicated otherwise the following examples were synthesized in analogy to the synthesis described for Examples 42 and 43 using suitable building blocks, respectively.

| | | | Building blocks | |
|---|---|---|---|---|
| | | | Chromatography | |
| | Systematic | | parameters | MS |
| Ex. | Name | Structure | Order of elution | m/z |
| 48 | (+)- or (−)-[6- [(2,4- Difluorophenyl) methyl]-2- azaspiro[3.3]heptan- 2-yl]-[3-(1H- 1,2,4-triazol-5- yl)pyrrolidin-1- yl]methanone | 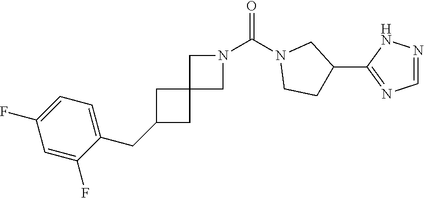 [SMILES: O=C(N1CCC(c2ncn[nH]2)C1)N1CC2(CC(Cc3ccc(F)cc3F)C2)C1] | 6-[(2,4- Difluorophenyl) methyl]-2- azaspiro[3.3]heptane 2,2,2-trifluoroacetic acid (BB10) and 5- Pyrrolidin-3-yl-4H- 1,2,4-triazole 1909319-47-6) HPLC Method AD first eluting | 388.3 [M + H]⁺ |
| 49 | (−)- or (+)-[6- [(2,4- Difluorophenyl) methyl]-2- azaspiro[3.3] heptan-2-yl]-[3- (1H-1,2,4-triazol-5- yl)pyrrolidin-1- yl]methanone | 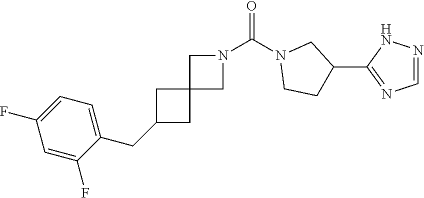 [SMILES: O=C(N1CCC(c2ncn[nH]2)C1)N1CC2(CC(Cc3ccc(F)cc3F)C2)C1] | 6-[(2,4- Difluorophenyl) methyl]-2- azaspiro[3.3]heptane 2,2,2-trifluoroacetic acid (BB10) and 5- Pyrrolidin-3-yl-4H- 1,2,4-triazole 1909319-47-6) HPLC Method AD second eluting | 388.3 [M + H]⁺ |
| 51 | (+)- or (−)-[2-[2- Fluoro-4- (trifluoromethyl) phenoxy]-7- azaspiro [3.5] nonan-7-yl]-[3- (1H-1,2,4-triazol- 5-yl)pyrrolidin-1- yl]methanone | 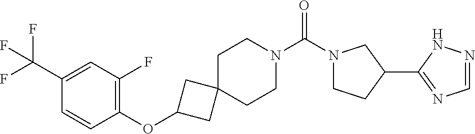 [SMILES: O=C(N1CCC2(CC1)CC(Oc1ccc(C(F)(F)F)cc1F)C2)N1CCC(c2ncn[nH]2)C1] | 2-[2-Fluoro-4- (trifluoromethyl) phenoxy]-7- azaspiro[3.5]nonane 2,2,2-trifluoroacetic acid (BB11) and 5- Pyrrolidin-3-yl-4H- 1,2,4-triazole 1909319-47-6) HPLC Method AD first eluting | 468.3 [M + H]⁺ |
| 52 | (−)- or (+)-[2-[2- Fluoro-4- (trifluoromethyl) phenoxy]-7- azaspiro[3.5] nonan-7-yl]-[3- (1H- 1,2,4-triazol-5- yl)pyrrolidin-1- yl]methanone | 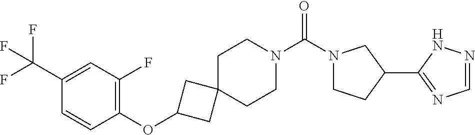 [SMILES: O=C(N1CCC2(CC1)CC(Oc1ccc(C(F)(F)F)cc1F)C2)N1CCC(c2ncn[nH]2)C1] | 2-[2-Fluoro-4- (trifluoromethyl) phenoxy]-7- azaspiro[3.5]nonane 2,2,2-trifluoroacetic acid (BB11) and 5- Pyrrolidin-3-yl-4H- 1,2,4-triazole 1909319-47-6) HPLC Method AD second eluting | 468.3 [M + H]⁺ |
| 60 | (+)- or (−)-[3-[2- [2-Fluoro-4- (trifluoromethyl) phenyl]ethyl] azetidin-1-yl]-[3- (1H-1,2,4-triazol- 5-yl)pyrrolidin-1- yl]methanone | 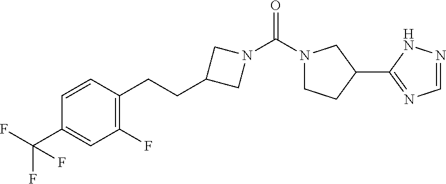 [SMILES: O=C(N1CC(CCc2ccc(C(F)(F)F)cc2F)C1)N1CCC(c2ncn[nH]2)C1] | 3-[2-[2-Fluoro-4- (trifluoromethyl) phenyl]ethyl] azetidine 4- methylbenzenesulfonic acid (CAS RN 2411577-40-5) and 5- Pyrrolidin-3-yl-4H- 1,2,4-triazole hydrochloride (CAS RN 1909337-56-9) HPLC Method AD first eluting | 412.3 [M + H]⁺ |
| 61 | (−)- or (+)-[3-[2- [2-Fluoro-4- (trifluoromethyl) phenyl]ethyl] azetidin-1-yl]-[3- (1H-1,2,4-triazol- 5-yl)pyrrolidin-1- yl]methanone | 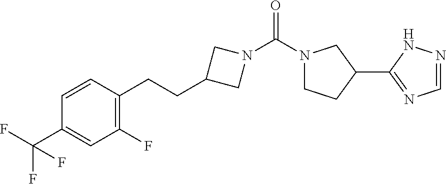 [SMILES: O=C(N1CC(CCc2ccc(C(F)(F)F)cc2F)C1)N1CCC(c2ncn[nH]2)C1] | 3-[2-[2-Fluoro-4- (trifluoromethyl) phenyl]ethyl] azetidine 4- methylbenzenesulfonic acid (CAS RN 2411577-40-5) and 5- Pyrrolidin-3-yl-4H- 1,2,4-triazole hydrochloride (CAS RN 1909337-56-9) HPLC Method AD second eluting | 412.3 [M + H]⁺ |
| 67 | (+)- or (−)-1-[3- [[2-Fluoro-4- (trifluoromethyl) phenyl]methoxy] azetidine-1- carbonyl]-N- methyl- pyrrolidine-3- carboxamide | 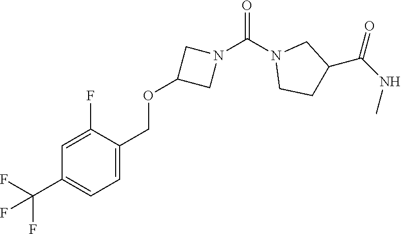 [SMILES: CNC(=O)C1CCN(C(=O)N2CC(OCc3ccc(C(F)(F)F)cc3F)C2)C1] | 3-((2-Fluoro-4- (trifluoromethyl) benzyl)oxy) azetidine 4- methylbenzenesulfonic acid (CAS RN 2135785-94-1) and N-methylpyrrolidine- 3-carboxamide hydrochloride (CAS RN 1007870-02-1) Chiral SFC Method IC first eluting | 404.2 [M + H]⁺ |
| 68 | (−)- or (+)-(3R)- 1-[3-[[2-Fluoro- 4- (trifluoromethyl) phenyl]methoxy] azetidine-1- carbonyl]-N- methyl- pyrrolidine-3- carboxamide | 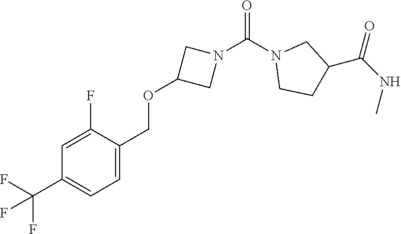 [SMILES: CNC(=O)C1CCN(C(=O)N2CC(OCc3ccc(C(F)(F)F)cc3F)C2)C1] | 3-((2-Fluoro-4- (trifluoromethyl) benzyl)oxy)azetidine 4- methylbenzenesulfonic acid (CAS RN 2135785-94-1) and N-methylpyrrolidine- 3-carboxamide hydrochloride (CAS RN 1007870-02-1) SFC Method IC second eluting | 404.2 [M + H]⁺ |
| 69 | (+)- or (−)-1-[3- [[2-Fluoro-4- (trifluoromethyl) phenyl]methoxy] azetidine-1- carbonyl]-N,N- dimethyl- pyrrolidine-3- carboxamide | 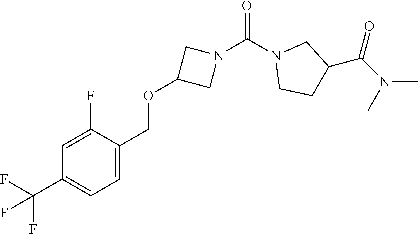 [SMILES: CN(C)C(=O)C1CCN(C(=O)N2CC(OCc3ccc(C(F)(F)F)cc3F)C2)C1] | 3-((2-Fluoro-4- (trifluoromethyl) benzyl)oxy)azetidine 4- methylbenzenesulfonic acid (CAS RN 2135785-94-1) and N,N- dimethylpyrrolidine- 3-carboxamide hydrochloride (CAS RN 1211573-00-0) SFC Method ADH1 first eluting | 418.4 [M + H]⁺ |
| 70 | (−)- or (+)-1-[3- [[2-Fluoro-4- (trifluoromethyl) phenyl]methoxy] azetidine-1- carbonyl]-N,N- dimethyl- pyrrolidine-3- carboxamide | 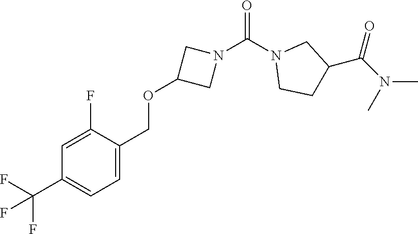 [SMILES: CN(C)C(=O)C1CCN(C(=O)N2CC(OCc3ccc(C(F)(F)F)cc3F)C2)C1] | 3-((2-Fluoro-4- (trifluoromethyl) benzyl)oxy)azetidine 4- methylbenzenesulfonic acid (CAS RN 2135785-94-1) and N,N- dimethylpyrrolidine- 3-carboxamide hydrochloride (CAS RN 1211573-00-0) SFC Method ADH1 second eluting | 418.4 [M + H]⁺ |
| 71 | (+)- or (−)-[3-[[2- Fluoro-4- (trifluoromethyl) phenyl]methoxy] azetidin-1-yl]-[3- pyrazol-1- ylpyrrolidin-1- yl]methanone | 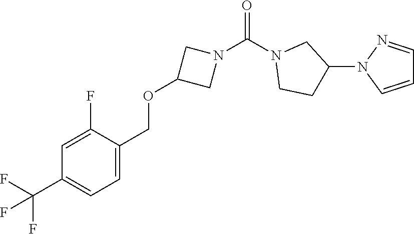 [SMILES: O=C(N1CC(OCc2ccc(C(F)(F)F)cc2F)C1)N1CCC(n2cccn2)C1] | 3-((2-Fluoro-4- (trifluoromethyl) benzyl)oxy)azetidine 4- methylbenzenesulfonic acid (CAS RN 2135785-94-1) and 1- Pyrrolidin-3- ylpyrazole dihydrochloride (CAS RN 1242339- 08-7) SFC Method IA first eluting | 413.3 [M + H]⁺ |
| 72 | (−)- or (+)-[3-[[2- Fluoro-4- (trifluoromethyl) phenyl]methoxy] azetidin-1-yl]-[3- pyrazol-1- ylpyrrolidin-1- yl]methanone | 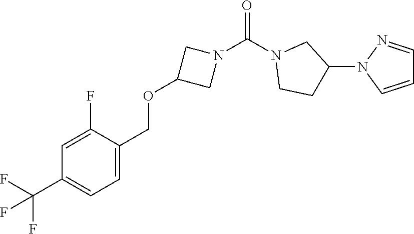 [SMILES: O=C(N1CC(OCc2ccc(C(F)(F)F)cc2F)C1)N1CCC(n2cccn2)C1] | 3-((2-Fluoro-4- (trifluoromethyl) benzyl)oxy)azetidine 4- methylbenzenesulfonic acid (CAS RN 2135785-94-1) and 1- Pyrrolidin-3- ylpyrazole dihydrochloride (CAS RN 1242339- 08-7) SFC Method IA second eluting | 413.4 [M + H]⁺ |
| 73 | (+)- or (−)-1-[3- [4-(2,4- Difluorophenyl) phenyl]azetidine- 1-carbonyl] pyrrolidine-3- carboxamide | 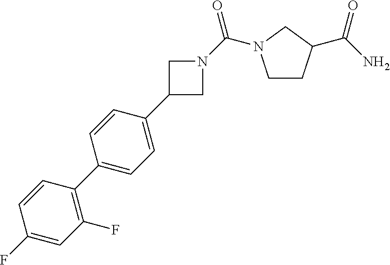 [SMILES: NC(=O)C1CCN(C(=O)N2CC(c3ccc(-c4ccc(F)cc4F)cc3)C2)C1] | 3-[4-(2,4- Difluorophenyl) phenyl]azetidine 4- methylbenzenesulfonic acid (BB12) and Pyrrolidine-3- carboxamide 2,2,2- trifluoroacetic acid (CAS RN1673553- 81-5) SFC Method OJH first eluting | 386.3 [M + H]⁺ |
| 74 | (−)- or (+)-1-[3- [4-(2,4- Difluorophenyl) phenyl]azetidine- 1-carbonyl] pyrrolidine-3- carboxamide | 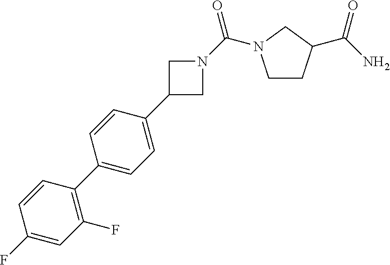 [SMILES: NC(=O)C1CCN(C(=O)N2CC(c3ccc(-c4ccc(F)cc4F)cc3)C2)C1] | 3-[4-(2,4- Difluorophenyl) phenyl]azetidine 4- methylbenzenesulfonic acid (BB12) and Pyrrolidine-3- carboxamide 2,2,2- trifluoroacetic acid (CAS RN1673553- 81-5) SFC Method OJH second eluting | 386.3 [M + H]⁺ |
| 86 | (−)- or (+)-[3-[4- (2,4- Difluorophenyl) phenyl]azetidin-1- yl]-[3-(1H-1,2,4- triazol-5- yl)pyrrolidin-1- yl]methanone | 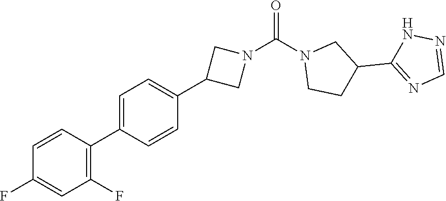 [SMILES: O=C(N1CC(c2ccc(-c3ccc(F)cc3F)cc2)C1)N1CCC(c2ncn[nH]2)C1] | 3-[4-(2,4- Difluorophenyl) phenyl]azetidine 4- methylbenzenesulfonic acid (BB12) and 5-Pyrrolidin-3-yl-4H- 1,2,4-triazole dihydrochloride (CAS RN 1909319- 47-6) HPLC Method NR second eluting | 410.3 [M + H]⁺ |
| 87 | (+)- or (−)-[3-[4- (2,4- Difluorophenyl) phenyl]azetidin-1- yl]-[3-(1H-1,2,4- triazol-5- yl)pyrrolidin-1- yl]methanone | 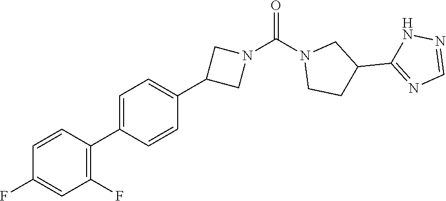 [SMILES: O=C(N1CC(c2ccc(-c3ccc(F)cc3F)cc2)C1)N1CCC(c2ncn[nH]2)C1] | 3-[4-(2,4- Difluorophenyl) phenyl]azetidine 4- methylbenzenesulfonic acid (BB12) and 5-Pyrrolidin-3-yl-4H- 1,2,4-triazole dihydrochloride (CAS RN 1909319- 47-6) HPLC Method NR first eluting | 410.3 [M + H]⁺ |
| 90 | 4-[ 1-[3-[3- Chloro-4- (trifluoromethoxy) phenyl]azetidine- 1- carbonyl] pyrrolidin-3- yl]oxazolidin-2- one | 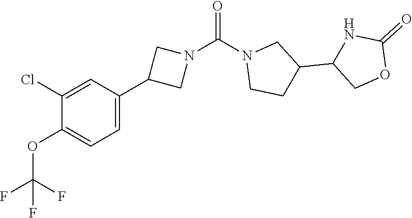 [SMILES: O=C1NC(C2CCN(C(=O)N3CC(c4ccc(OC(F)(F)F)c(Cl)c4)C3)C2)CO1] | 3-[3-Chloro-4- (trifluoromethoxy) phenyl]azetidine (CAS RN 1260891-17-5) and 4-Pyrrolidin-3- yloxazolidin-2-one (CAS RN 1888977- 61-4) HPLC Method OD2 first eluting, mixture of 2 isomers | 434.3 [M + H]⁺ |
| 91 | 4-[ 1-[3-[3- Chloro-4- (trifluoromethoxy) phenyl]azetidine- 1-carbonyl] pyrrolidin-3- yl]oxazolidin-2- one | 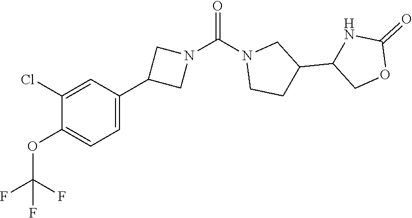 [SMILES: O=C1NC(C2CCN(C(=O)N3CC(c4ccc(OC(F)(F)F)c(Cl)c4)C3)C2)CO1] | 3-[3-Chloro-4- (trifluoromethoxy) phenyl]azetidine (CAS RN 1260891-17-5) and 4-Pyrrolidin-3- yloxazolidin-2-one (CAS RN 1888977- 61-4) HPLC Method OD2 second eluting, mixture of 2 isomers | 434.3 [M + H]⁺ |
| 100 | 4-[ 1-[3-[4- (2,2,2- Trifluoroethyl) phenyl]azetidine-1- carbonyl] pyrrolidin-3- yl]oxazolidin-2- one | 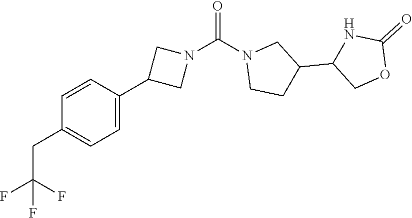 [SMILES: O=C1NC(C2CCN(C(=O)N3CC(c4ccc(CC(F)(F)F)cc4)C3)C2)CO1] | 3-[4-(2,2,2- Trifluoroethyl)phenyl Jazetidine (CAS RN 2377009-78-2) and 4- Pyrrolidin-3- yloxazolidin-2-one (CAS RN 1888977- 61-4) HPLC Method GN second eluting, mixture of 2 isomers | 398.3 [M + H]⁺ |
| 101 | 4-[ 1-[3-[4- (2,2,2- Trifluoroethyl) phenyl]azetidine-1- carbonyl] pyrrolidin-3- yl]oxazolidin-2- one | 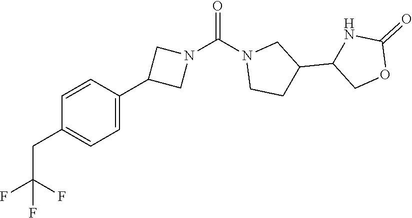 [SMILES: O=C1NC(C2CCN(C(=O)N3CC(c4ccc(CC(F)(F)F)cc4)C3)C2)CO1] | 3-[4-(2,2,2- Trifluoroethyl)phenyl Jazetidine (CAS RN 2377009-78-2) and 4- Pyrrolidin-3- yloxazolidin-2-one (CAS RN 1888977- 61-4) HPLC Method GN first eluting, mixture of 2 isomers | 398.3 [M + H]⁺ |
| 105 | 4-[1-[3-[4-(2,4- Difluorophenyl) phenyl]azetidine- 1-carbonyl] pyrrolidin-3- yl]oxazolidin-2- one | 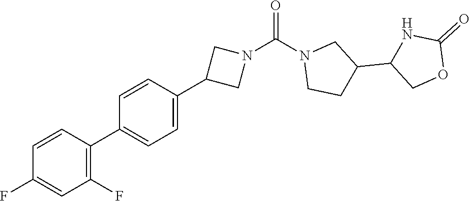 [SMILES: O=C1NC(C2CCN(C(=O)N3CC(c4ccc(-c5ccc(F)cc5F)cc4)C3)C2)CO1] | 3-[4-(2,4- Difluorophenyl) phenyl]azetidine 4- methylbenzenesulfonic acid (BB12) and 4-Pyrrolidin-3- yloxazolidin-2-one (CAS RN 1888977- 61-4) SFC Method OJH1 first eluting, mixture of 2 isomers | 428.3 [M + H]⁺ |
| 106 | 4-[1-[3-[4-(2,4- Difluorophenyl) phenyl]azetidine- 1-carbonyl] pyrrolidin-3- yl]oxazolidin-2- one | 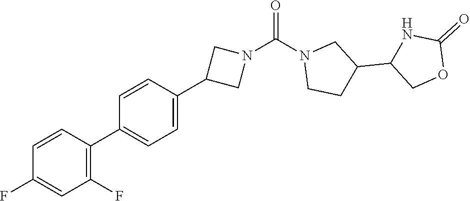 [SMILES: O=C1NC(C2CCN(C(=O)N3CC(c4ccc(-c5ccc(F)cc5F)cc4)C3)C2)CO1] | 3-[4-(2,4- Difluorophenyl)phen yl]azetidine 4- methylbenzenesulfonic acid (BB12) and 4-Pyrrolidin-3- yloxazolidin-2-one (CAS RN 1888977- 61-4) SFC Method OJH1 second eluting, mixture of 2 isomers | 428.3 [M + H]⁺ |
| 109 | 4-[1-[3-[4-[1- (Trifluoromethyl) cyclopropyl] phenyl]azetidine- 1-carbonyl] pyrrolidin-3- yl]oxazolidin-2- one | 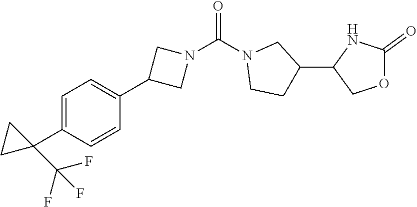 [SMILES: O=C1NC(C2CCN(C(=O)N3CC(c4ccc(C5(C(F)(F)F)CC5)cc4)C3)C2)CO1] | 3-[4-[1- (Trifluoromethyl) cyclopropyl]phenyl] azetidine 4- methylbenzenesulfonic acid (BB9) and 4- Pyrrolidin-3- yloxazolidin-2-one (CAS RN 1888977- 61-4) 1) HPLC Method NR second eluting together with 110 + 111 2) SFC Method ODH first eluting | 424.3 [M + H]⁺ |
| 110 | 4-[1-[3-[4-[1- (Trifluoromethyl) cyclopropyl] phenyl]azetidine-1- carbonyl] pyrrolidin-3- yl]oxazolidin-2- one | 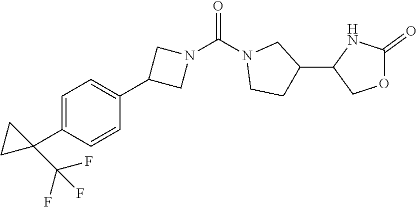 [SMILES: O=C1NC(C2CCN(C(=O)N3CC(c4ccc(C5(C(F)(F)F)CC5)cc4)C3)C2)CO1] | 3-[4-[1- (Trifluoromethyl) cyclopropyl]phenyl] azetidine 4- methylbenzenesulfonic acid (BB9) and 4- Pyrrolidin-3- yloxazolidin-2-one (CAS RN 1888977- 61-4) 1)HPLC Method NR second eluting together with 109 + 111 2)SFC Method ODH second eluting | 424.3 [M + H]⁺ |
| 111 | 4-[1-[3-[4-[1- (Trifluoromethyl) cyclopropyl] phenyl]azetidine-1- carbonyl] pyrrolidin-3- yl]oxazolidin-2- one | 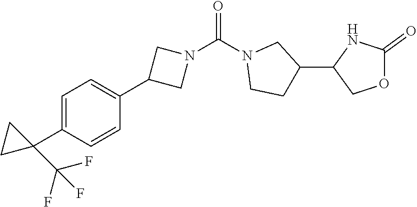 [SMILES: O=C1NC(C2CCN(C(=O)N3CC(c4ccc(C5(C(F)(F)F)CC5)cc4)C3)C2)CO1] | 3-[4-[1- (Trifluoromethyl) cyclopropyl]phenyl] azetidine 4- methylbenzenesulfonic acid (BB9) and 4- Pyrrolidin-3- yloxazolidin-2-one (CAS RN 1888977- 61-4) 1)HPLC Method NR second eluting together with 109 + 110 2)SFC Method ODH third eluting | 424.3 [M + H]⁺ |
| 118 | 4-[1-[3-[4-[1- (Trifluoromethyl) cyclopropyl] phenyl]azetidine-1- carbonyl] pyrrolidin-3- yl]oxazolidin-2- one | 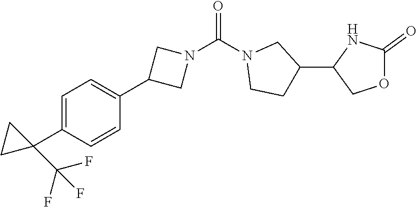 [SMILES: O=C1NC(C2CCN(C(=O)N3CC(c4ccc(C5(C(F)(F)F)CC5)cc4)C3)C2)CO1] | 3-[4-[1- (Trifluoromethyl) cyclopropyl]phenyl] azetidine 4- methylbenzenesulfonic acid (BB9) and 4- Pyrrolidin-3- yloxazolidin-2-one (CAS RN 1888977- 61-4) HPLC Method NR first eluting | 424.3 [M + H]⁺ |
| 125 | (+)- or (−)-[3- (1H-1,2,4- Triazol-5- yl)pyrrolidin-1- yl]-[3-[4-(2,2,2- trifluoro-1,1- dimethyl- ethoxy)phenyl] azetidin-1- yl]methanone | 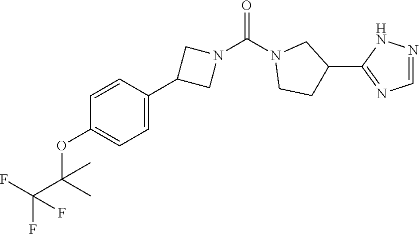 [SMILES: CC(C)(Oc1ccc(C2CN(C(=O)N3CCC(c4ncn[nH]4)C3)C2)cc1)C(F)(F)F] | 3-(4-((1,1,1- Trifluoro-2- methylpropan-2- yl)oxy)phenyl) azetidine 4- methylbenzenesulfonic acid (BB24) and 5- Pyrrolidin-3-yl-4H- 1,2,4-triazole dihydrochloride (CAS RN 1909319- 47-6) HPLC Method AD first eluting | 424.3 [M + H]⁺ |
| 126 | (−)- or (+)-[3- (1H-1,2,4- Triazol-5- yl)pyrrolidin-1- yl]-[3-[4-(2,2,2- trifluoro-1,1- dimethyl- ethoxy)phenyl] azetidin-1- yl]methanone | 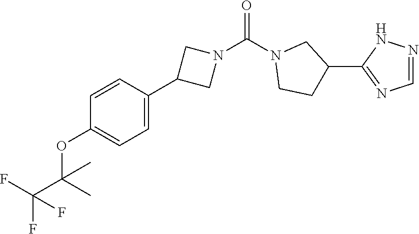 [SMILES: CC(C)(Oc1ccc(C2CN(C(=O)N3CCC(c4ncn[nH]4)C3)C2)cc1)C(F)(F)F] | 3-(4-((1,1,1- Trifluoro-2- methylpropan-2- yl)oxy)phenyl) azetidine 4- methylbenzenesulfonic acid (BB24) and 5- Pyrrolidin-3-yl-4H- 1,2,4-triazole dihydrochloride (CAS RN 1909319- 47-6) HPLC Method AD first eluting | 424.3 [M + H]⁺ |

### Example 83

### trans-[3-[[2-Fluoro-4-(trifluoromethyl)phenyl]methoxy]cyclobutyl]-[3-(1H-triazol-5-yl)pyrrolidin-1-yl]methanone

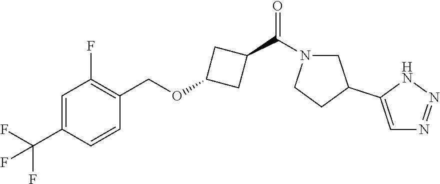 [SMILES: O=C([C@H]1C[C@H](OCc2ccc(C(F)(F)F)cc2F)C1)N1CCC(c2cnn[nH]2)C1]

To a solution of trans-3-[2-Fluoro-4-(trifluoromethyl)phenyl]methoxy]cyclobutanecarboxylic acid (50 mg, 132 μmol, BB13) and 5-(pyrrolidin-3-yl)-1H-1,2,3-triazole (18.2 mg, 132 μmol, CAS RN 1517602-91-3) in DMF (0.34 mL) were added HATU (55 mg, 145 μmol) and DIPEA (92 μL, 0.53 mmol) and the mixture was stirred at RT overnight. The product was purified by HPLC Method GN to get the desired compound as a light yellow oil (0.036 g; 50.5%). MS (ESI): m/z=412.2 [M+H]⁺.

### Example 821

### [(3S)-3-(1H-1,2,4-Triazol-5-yl)pyrrolidin-1-yl]-[4-[[5-(trifluoromethyl)pyrazin-2-yl]amino]cuban-1-yl]methanone

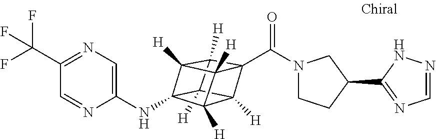 [SMILES: O=C(N1CC[C@H](c2ncn[nH]2)C1)C12[C@H]3[C@H]4[C@@H]1[C@H]1[C@@H]2[C@@H]3[C@@]41Nc1cnc(C(F)(F)F)cn1]

The product was obtained in analogy to example 1 starting from 5-[(3S)-pyrrolidin-3-yl]-1H-1,2,4-triazole (CAS RN 2165724-79-6) and 4-[5-(trifluoromethyl)pyrazin-2-yl]amino]cubane-1-carboxylic acid (BB84) to get the title compound as a colorless solid. MS (ESI): m/z=430.1 [M+H]⁺.

If not indicated otherwise the following examples were synthesized in analogy to the synthesis described for Example 83 using suitable building blocks, respectively.

| | | | Building blocks | |
|---|---|---|---|---|
| | | | Chromatography | |
| | | | parameters | MS |
| Ex. | Systematic Name | Structure | Order of elution | m/z |
| 168 | [(3R)-3-(1H- Triazol-5- yl)pyrrolidin-1- yl]-[2-(3,3,3- trifluoropropoxy) spiro[3.3]heptan- 6-yl]methanone | 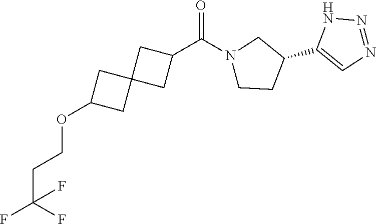 [SMILES: O=C(C1CC2(CC(OCCC(F)(F)F)C2)C1)N1CC[C@@H](c2cnn[nH]2)C1] | Lithium 2-(3,3,3- trifluoropropoxy)spiro [3.3]heptane-6- carboxylate (BB38) and 5-[(3R)- Pyrrolidin-3-yl]-1H- triazole (CAS RN 2166044-57-9) | 371.2 [M − H]⁻ |
| 181 | [3-[[2-Fluoro-4- (trifluoromethyl) phenyl]methoxy] azetidin-1-yl]-[3- (1H-pyrazol-5- yl)phenyl] methanone | 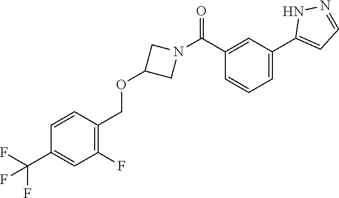 [SMILES: O=C(c1cccc(-c2ccn[nH]2)c1)N1CC(OCc2ccc(C(F)(F)F)cc2F)C1] | 3-(1H-Pyrazol-5- yl)benzoic acid (CAS RN 850375-11-0) and 3-((2-Fluoro-4- (trifluoromethyl) benzyl)oxy)azetidine (CAS RN 1121597- 80-5) HPLC Method YMC | 420.2 [M + H]⁺ |
| 186 | [(3S)-3-(1H- Triazol-5- yl)pyrrolidin-1- yl]-[2-(3,3,3- trifluoropropoxy) spiro[3.3]heptan- 6-yl]methanone | 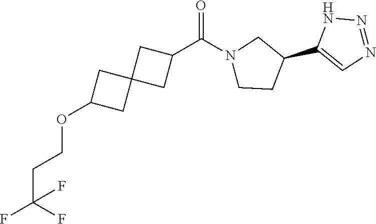 [SMILES: O=C(C1CC2(CC(OCCC(F)(F)F)C2)C1)N1CC[C@H](c2cnn[nH]2)C1] | Lithium 2-(3,3,3- trifluoropropoxy) spiro[3.3]heptane-6- carboxylate (BB38) and 5-[(3S)- Pyrrolidin-3-yl]-1H- triazole (CAS RN 2166205-35-0) HPLC Method GN | 373.1 [M + H]⁺ |
| 187 | [2-[[1- (Difluoromethyl) cyclopropyl] methoxy] spiro[3.3]heptan- 6-yl]-[(3S)-3- (1H-triazol-5- yl)pyrrolidin-1- | 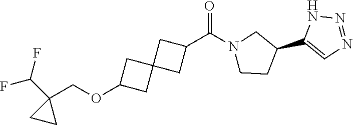 [SMILES: O=C(C1CC2(CC(OCC3(C(F)F)CC3)C2)C1)N1CC[C@H](c2cnn[nH]2)C1] | 2-[[1- (Difluoromethyl)cyclo propyl]methoxy]spiro [3.3]heptane-6- carboxylic acid (BB43) and 5-[(3S)- Pyrrolidin-3-yl]-1H- triazole (CAS RN | 384.2 [M + H]⁺ |
| | yl]methanone | | 2166205-35-0) | |
| 188 | [8-(3- Cyclopropyl- 1,2,4-oxadiazol- 5-yl)-8- azabicyclo[3.2.1] octan-3-yl]- [(3S)-3-(1H- triazol-5- yl)pyrrolidin-1- yl]methanone | 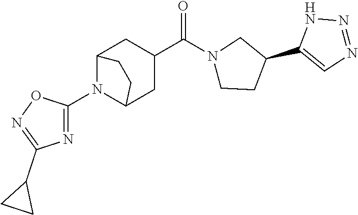 [SMILES: O=C(C1CC2CCC(C1)N2c1nc(C2CC2)no1)N1CC[C@H](c2cnn[nH]2)C1] | 8-(3-Cyclopropyl- 1,2,4-oxadiazol-5-yl)- 8- azabicyclo[3.2.1] octane-3-carboxylic acid (BB42) and 5-[(3S)- Pyrrolidin-3-yl]-1H- triazole (CAS RN 2166205-35-0) HPLC Method YMC | 384.2 [M + H]⁺ |
| 460 | 3-[3-[4-(4- Chloro-2- methylsulfonyl- phenyl)phenyl] azetidine-1- carbonyl]-4,5- dihydroisoxazole- 5-carboxamide | 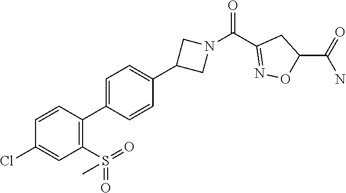 [SMILES: CS(=O)(=O)c1cc(Cl)ccc1-c1ccc(C2CN(C(=O)C3=NOC(C(N)=O)C3)C2)cc1] | 3-[4-(4-Chloro-2- methylsulfonyl- phenyl)phenyl] azetidine;4- methylbenzenesulfonic acid (BB44) and 5- Carbamoyl-4,5- dihydroisoxazole-3- carboxylic acid (BB70) HPLC Method GN | 461.2 [M + H]⁺ |

### Example 92

### [3-[4-(2-Methylazetidin-1-yl)phenyl]azetidin-1-yl]-[(3S or 3R)-3-(1H-pyrazol-5-yl)pyrrolidin-1-yl]methanone

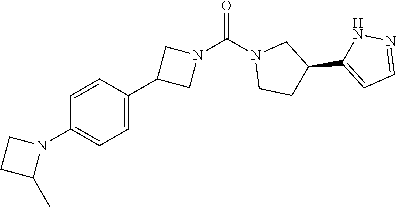 [SMILES: CC1CCN1c1ccc(C2CN(C(=O)N3CC[C@H](c4ccn[nH]4)C3)C2)cc1]

A solution of 5-[(3S or 3R)-pyrrolidin-3-yl]-1H-pyrazole (38.0 mg, 0.280 mmol, BB25), (4-nitrophenyl) 3-[4-(2-methylazetidin-1-yl)phenyl]azetidine-1-carboxylate (50.0 mg, 0.140 mmol, BB20) and DIPEA (0.06 mL, 0.340 mmol) in ACN (3 mL) was stirred at 25° C. for 16 hours. The solution was concentrated in vacuo to give a residue, which was purified by on a preparative HPLC (Gemini NX column) using a gradient of acetonitrile:water (containing 0.1% formic acid)(18:82 to 100:0). The product was purified on a preparative HPLC (Xtimate C₁₈ column) using a gradient of acetonitrile:water (containing 0.05% NH3·H2O)(27:73 to 57:43) to give the desired product as a colorless oil (9 mg, 18.1%). MS (ESI): m/z=366.2 [M+H]⁺. If not indicated otherwise the following examples were synthesized in analogy to the synthesis described for Example 92 using suitable building blocks, respectively.

| | | | | MS |
|---|---|---|---|---|
| Ex. | Systematic Name | Structure | Building blocks | m/z |
| 93 | [3-[4-(2- Methylazetidin- 1- yl)phenyl]azetidin- 1-yl]-[(3R or 3S)-3-(1H- pyrazol-5- yl)pyrrolidin-1- yl]methanone | 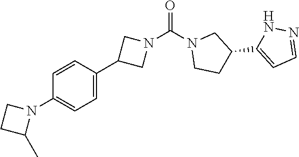 [SMILES: CC1CCN1c1ccc(C2CN(C(=O)N3CC[C@@H](c4ccn[nH]4)C3)C2)cc1] | (4-Nitrophenyl) 3-[4- (2-methylazetidin-1- yl)phenyl]azetidine- 1-carboxylate (BB20) and 5-[(3R or 3S)- Pyrrolidin-3-yl]-1H- pyrazole (BB26) | 366.2 [M + H]⁺ |
| 94 | [3-[4-(3- Fluoropyrrolidin- 1- yl)phenyl]azetidin- 1-yl]-[(3R or 3S)-3-(1H- pyrazol-5- yl)pyrrolidin-1- yl]methanone | 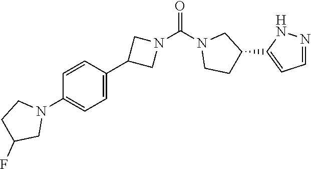 [SMILES: O=C(N1CC(c2ccc(N3CCC(F)C3)cc2)C1)N1CC[C@@H](c2ccn[nH]2)C1] | (4-Nitrophenyl) 3-[4- (3-fluoropyrrolidin-1- yl)phenyl]azetidine- 1-carboxylate (BB21) and 5-[(3R or 3S)- Pyrrolidin-3-yl]-1H- pyrazole (BB26) | 384.1 [M + H]⁺ |
| 99 | [3-[4-(3- Fluoropyrrolidin- 1- yl)phenyl]azetidin- 1-yl]-[(3S or 3R)-3-(1H- pyrazol-5- yl)pyrrolidin-1- yl]methanone | 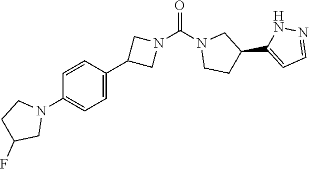 [SMILES: O=C(N1CC(c2ccc(N3CCC(F)C3)cc2)C1)N1CC[C@H](c2ccn[nH]2)C1] | (4-Nitrophenyl) 3-[4- (3-fluoropyrrolidin-1- yl)phenyl]azetidine- 1-carboxylate (BB21) and 5-[(3S or 3R)- Pyrrolidin-3-yl]-1H- pyrazole (BB25) | 384.1 [M + H]⁺ |
| 102 | [3-[4-(Oxetan-3- yl)phenyl]azetidin- 1-yl]-[3-(1H- pyrazol-5- yl)pyrrolidin-1- yl]methanone | 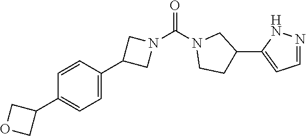 [SMILES: O=C(N1CC(c2ccc(C3COC3)cc2)C1)N1CCC(c2ccn[nH]2)C1] | (4-Nitrophenyl) 3-[4- (oxetan-3- yl)phenyl]azetidine- 1-carboxylate (BB22) and 5-(Pyrrolidin-3- yl)-1H-pyrazole hydrochloride (CAS RN 1956389-94-8) | 353.2 [M + H]⁺ |
| 127 | (+)- or (−)-[4-[[2- Chloro-4- (trifluoromethyl) phenyl]methoxy]- 1-piperidyl]-[(3S or 3R)-3-(1H- triazol-5- yl)pyrrolidin-1- yl]methanone | 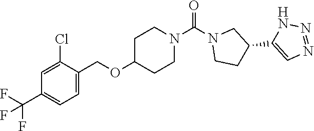 [SMILES: O=C(N1CCC(OCc2ccc(C(F)(F)F)cc2Cl)CC1)N1CC[C@@H](c2cnn[nH]2)C1] | (4-Nitrophenyl) (3S or 3R)-3-(1H-triazol- 5-yl)pyrrolidine-1- carboxylate (BB28) and 4-[[2-Chloro-4- (trifluoromethyl)phenyl] methoxy]piperidine 2,2,2-trifluoroacetic acid (BB30) | 458.1 [M + H]⁺ |
| 128 | (−)- or (+)-[4-[[2- Chloro-4- (trifluoromethyl) phenyl]methoxy]- 1-piperidyl]- [(3R or 3S)-3- (1H-triazol-5- yl)pyrrolidin-1- yl]methanone | 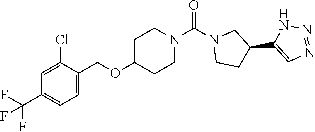 [SMILES: O=C(N1CCC(OCc2ccc(C(F)(F)F)cc2Cl)CC1)N1CC[C@H](c2cnn[nH]2)C1] | (4-Nitrophenyl) (3R or 3S)-3-(1H-triazol- 5-yl)pyrrolidine-1- carboxylate (BB29) and 4-[[2-Chloro-4- (trifluoromethyl)phenyl] methoxy]piperidine 2,2,2-trifluoroacetic acid (BB30) | 458.1 [M + H]⁺ |
| 129 | [(3S or 3R)-3- (1H-Triazol-5- yl)pyrrolidin-1- yl]-[3-[[4- (trifluoromethyl) phenyl]methyl] pyrrolidin-1- yl]methanone | 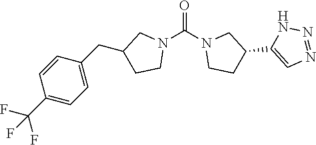 [SMILES: O=C(N1CCC(Cc2ccc(C(F)(F)F)cc2)C1)N1CC[C@@H](c2cnn[nH]2)C1] | (4-Nitrophenyl) (3S or 3R)-3-(1H-triazol- 5-yl)pyrrolidine-1- carboxylate (BB28) and 3-[[4- (Trifluoromethyl)phenyl] methyl]pyrrolidine (CAS RN 957998- 84-4) | 394.2 [M + H]⁺ |
| 130 | [3-[[2-Fluoro-4- (trifluoromethyl) phenyl]methoxy]- 8- azabicyclo[3.2.1] octan-8-yl]-[(3S or 3R)-3-(1H- triazol-5- yl)pyrrolidin-1- | 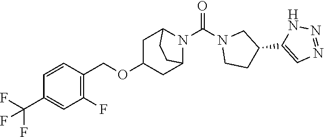 [SMILES: O=C(N1CC[C@@H](c2cnn[nH]2)C1)N1C2CCC1CC(OCc1ccc(C(F)(F)F)cc1F)C2] | (4-Nitrophenyl) (3S or 3R)-3-(1H-triazol- 5-yl)pyrrolidine-1- carboxylate (BB28) and 3-[[2-fluoro-4- (trifluoromethyl)phenyl] methoxy]-8- azabicyclo[3.2.1]octane 2,2,2- | 468.2 [M + H]⁺ |
| | yl]methanone | | trifluoroacetic acid | |
| | | | (BB31) | |
| 131 | [3-[[2-Fluoro-4- (trifluoromethyl) phenyl]methoxy]- 8- azabicyclo[3.2.1] octan-8-yl]-[(3R or 3S)-3-(1H- triazol-5- yl)pyrrolidin-1- | 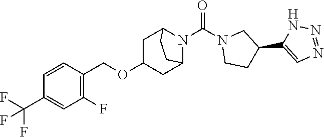 [SMILES: O=C(N1CC[C@H](c2cnn[nH]2)C1)N1C2CCC1CC(OCc1ccc(C(F)(F)F)cc1F)C2] | (4-Nitrophenyl) (3R or 3S)-3-(1H-triazol- 5-yl)pyrrolidine-1- carboxylate (BB29) and 3-[[2-fluoro-4- (trifluoromethyl)phenyl] methoxy]-8- azabicyclo[3.2.1]octane 2,2,2- | 468.3 [M + H]⁺ |
| | yl]methanone | | trifluoroacetic acid | |
| | | | (BB31) | |
| 132 | [(3aS,6aR)-5-[2- Fluoro-4- (trifluoromethyl) phenoxy]- 3,3a,4,5,6,6a- hexahydro-1H- cyclopenta[c]pyrrol- 2-yl]-[(3S or 3R)-3-(1H- triazol-5- | 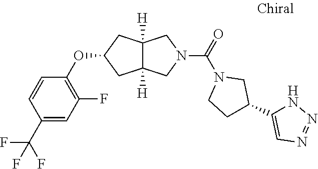 [SMILES: O=C(N1CC[C@@H](c2cnn[nH]2)C1)N1C[C@H]2C[C@H](Oc3ccc(C(F)(F)F)cc3F)C[C@H]2C1] | (4-Nitrophenyl) (3S or 3R)-3-(1H-triazol- 5-yl)pyrrolidine-1- carboxylate (BB28) and rac-(3aS,6aR)-5- [2-Fluoro-4- (trifluoromethyl) phenoxy]- 1,2,3,3a,4,5,6,6a- octahydrocyclopenta[c] | 454.2 [M + H]⁺ |
| | yl)pyrrolidin-1- | | pyrrole;2,2,2- | |
| | yl]methanone | | trifluoroacetic acid | |
| | | | (BB32) | |
| 133 | [(3aS,6aR)-5-[2- Fluoro-4- (trifluoromethyl) phenoxy]- 3,3a,4,5,6,6a- hexahydro-1H- cyclopenta[c]pyrrol- 2-yl]-[(3R or 3S)-3-(1H- triazol-5- | 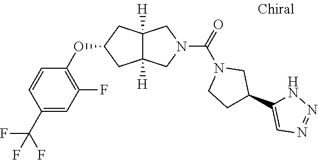 [SMILES: O=C(N1CC[C@H](c2cnn[nH]2)C1)N1C[C@H]2C[C@H](Oc3ccc(C(F)(F)F)cc3F)C[C@H]2C1] | (4-Nitrophenyl) (3R or 3S)-3-(1H-triazol- 5-yl)pyrrolidine-1- carboxylate (BB29) and rac-(3aS,6aR)-5- [2-Fluoro-4- (trifluoromethyl) phenoxy]- 1,2,3,3a,4,5,6,6a- octahydrocyclopenta[c] | 454.2 [M + H]⁺ |
| | yl)pyrrolidin-1- | | pyrrole;2,2,2- | |
| | yl]methanone | | trifluoroacetic acid | |
| | | | (BB32) | |
| 177 | [(3S)-3-[3- Chloro-5-(2,2,2- trifluoroethoxy) phenoxy]pyrrolidin- 1-yl]-[(3S or 3R)-3-(1H- triazol-5- yl)pyrrolidin-1- yl]methanone | 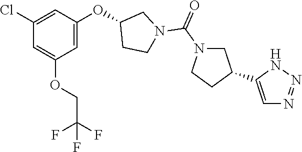 [SMILES: O=C(N1CC[C@H](Oc2cc(Cl)cc(OCC(F)(F)F)c2)C1)N1CC[C@@H](c2cnn[nH]2)C1] | (4-Nitrophenyl) (3S or 3R)-3-(1H-triazol- 5-yl)pyrrolidine-1- carboxylate (BB28) and (3S)-3-[3-chloro- 5-(2,2,2- trifluoroethoxy)phenoxy] pyrrolidine 2,2,2- trifluoroacetic acid | 460.1 [M + H]⁺ |
| | | | (BB40) | |
| 178 | [(3S)-3-[3- chloro-5-(2,2,2- trifluoroethoxy) phenoxy]pyrrolidin- 1-yl]-[(3R or 3S)-3-(1H- triazol-5- yl)pyrrolidin-1- yl]methanone | 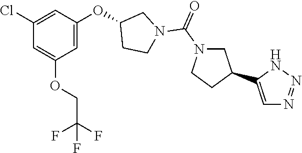 [SMILES: O=C(N1CC[C@H](Oc2cc(Cl)cc(OCC(F)(F)F)c2)C1)N1CC[C@H](c2cnn[nH]2)C1] | (4-Nitrophenyl) (3R or 3S)-3-(1H-triazol- 5-yl)pyrrolidine-1- carboxylate (BB29) and (3S)-3-[3-chloro- 5-(2,2,2- trifluoroethoxy)phenoxy] pyrrolidine 2,2,2- trifluoroacetic acid | 460.1 [M + H]⁺ |
| | | | (BB40) | |
| 179 | [(3R)-3-[3- Chloro-5-(2,2,2- trifluoroethoxy) phenoxy]pyrrolidin- 1-yl]-[(3S or 3R)-3-(1H- triazol-5- yl)pyrrolidin-1- yl]methanone | 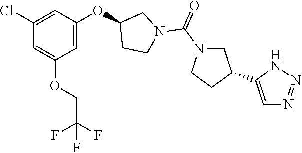 [SMILES: O=C(N1CC[C@@H](Oc2cc(Cl)cc(OCC(F)(F)F)c2)C1)N1CC[C@@H](c2cnn[nH]2)C1] | (4-Nitrophenyl) (3S or 3R)-3-(1H-triazol- 5-yl)pyrrolidine-1- carboxylate (BB28) and (3R)-3-[3-chloro- 5-(2,2,2- trifluoroethoxy)phenoxy] pyrrolidine 2,2,2- trifluoroacetic acid | 460.1 [M + H]⁺ |
| | | | (BB41) | |
| 180 | [(3R)-3-[3- Chloro-5-(2,2,2- trifluoroethoxy) phenoxy]pyrrolidin- 1-yl]-[(3R or 3S)-3-(1H- triazol-5- yl)pyrrolidin-1- yl]methanone | 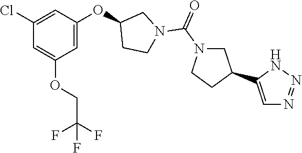 [SMILES: O=C(N1CC[C@@H](Oc2cc(Cl)cc(OCC(F)(F)F)c2)C1)N1CC[C@H](c2cnn[nH]2)C1] | (4-Nitrophenyl) (3R or 3S)-3-(1H-triazol- 5-yl)pyrrolidine-1- carboxylate (BB29) and (3R)-3-[3-chloro- 5-(2,2,2- trifluoroethoxy)phenoxy] pyrrolidine 2,2,2- trifluoroacetic acid | 460.1 [M + H]⁺ |
| | | | (BB41) | |

### Example 123

### (+)-[4-[(2-Chloro-4-fluoro-phenoxy)methyl]-1-piperidyl]-[(3R)-3-(1H-triazol-5-yl)pyrrolidin-1-yl]methanone and Example 124 (−)-[4-[(2-Chloro-4-fluoro-phenoxy)methyl]-1-piperidyl]-[(3S)-3-(1H-triazol-5-yl)pyrrolidin-1-yl]methanone

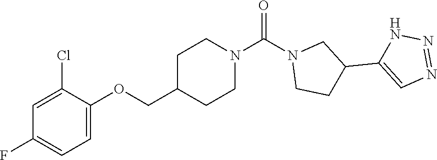 [SMILES: O=C(N1CCC(COc2ccc(F)cc2Cl)CC1)N1CCC(c2cnn[nH]2)C1]

A mixture of (4-nitrophenyl) 3-(1H-triazol-5-yl)pyrrolidine-1-carboxylate (305.19 mg, 1.01 mmol, BB27), DIPEA (649.85 mg, 5.03 mmol) and 4-[(2-chloro-4-fluoro-phenoxy)methyl]piperidine (204.0 mg, 0.840 mmol, CAS RN 883527-36-4) in ACN (6 mL) was stirred at 80° C. for 12 h. The reaction mixture was evaporated under reduced pressure to give the residue, which was then purified on a preparative HPLC (Gemini Luna C18 column) using a gradient of A:water (containing 0.225% FA) (20:80 to 98:2) and prep-TLC (DCM/MeOH=10/1) to give the racemate. The racemate was then separated by SFC Method AD5 to get the desired product example 123 (first eluting) as white solid (22.6 mg, 6.6% yield) MS (ESI): m/z=408.0 [M+H]⁺, and product example 124 (second eluting) as white solid (19.6 mg, 5.7% yield). MS (ESI): m/z=408.0 [M+H]⁺.

### Example 123. Specific Rotation: +21.10°

### Example 124. Specific Rotation: −17.03°

### Example 134

### (−)-[3-[[2-Fluoro-4-(trifluoromethyl)phenyl]methoxy]-2-methyl-azetidin-1-yl]-[(3S or 3R)-3-(1H-triazol-5-yl)pyrrolidin-1-yl]methanone and Example 135 (+)-[3-[[2-Fluoro-4-(trifluoromethyl)phenyl]methoxy]-2-methyl-azetidin-1-yl]-[(3S or 3R)-3-(1H-triazol-5-yl)pyrrolidin-1-yl]methanone

### Example 136

### (−)-[3-[[2-Fluoro-4-(trifluoromethyl)phenyl]methoxy]-2-methyl-azetidin-1-yl]-[(3S or 3R)-3-(1H-triazol-5-yl)pyrrolidin-1-yl]methanone and Example 137 (+)-[3-[[2-Fluoro-4-(trifluoromethyl)phenyl]methoxy]-2-methyl-azetidin-1-yl]-[(3S or 3R)-3-(1H-triazol-5-yl)pyrrolidin-1-yl]methanone

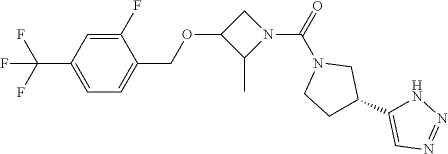 [SMILES: CC1C(OCc2ccc(C(F)(F)F)cc2F)CN1C(=O)N1CC[C@@H](c2cnn[nH]2)C1]

To a solution of 3-[[2-fluoro-4-(trifluoromethyl)phenyl]methoxy]-2-methyl-azetidine 2,2,2-trifluoroacetic acid (200.0 mg, 0.530 mmol, CAS RN2411576-99-1) and DIPEA (1.0 mL, 2.12 mmol) in ACN (15 mL) was added (4-nitrophenyl) (3S or 3R)-3-(1H-triazol-5-yl)pyrrolidine-1-carboxylate (176.86 mg, 0.580 mmol, BB28) and the mixture was stirred at 80° C. for 12 h. The reaction mixture was poured into H₂O (5 mL), extracted with EtOAc (5 mL*3), the organic layer was evaporated. The crude product was purified with Prep-HPLC (0.225% v/vFA) and lyophilized to give the racemate. The racemate was separated using SFC Method AD7 to get the desired product 136 as a white solid (18 mg, 7.7%) MS (ESI): m/z=428.1 [M+H]⁺ and the desired product 137 as a white solid (13 mg, 5.7%) MS (ESI): m/z=428.1 [M+H]⁺ along with an impure mixture of products 134 and 135.

This impure mixture was separated using SFC Method ADH3 to get the desired product 134 as a white solid (10.2 mg, 4.5%) MS (ESI): m/z=428.1 [M+H]⁺ and the desired product 135 as a white solid (10.6 mg, 4.7%) MS (ESI): m/z=428.1 [M+H]⁺.

### Example 134. Specific Rotation: −56.53°

### Example 135. Specific Rotation: +20.36°

### Example 136. Specific Rotation: −38.24°

### Example 137. Specific Rotation: +20.12°

### Example 138

### (+)-[3-[[2-Fluoro-4-(trifluoromethyl)phenyl]methoxy]-2-methyl-azetidin-1-yl]-[(3R or 3S)-3-(1H-triazol-5-yl)pyrrolidin-1-yl]methanone and Example 139 (−)-[3-[2-Fluoro-4-(trifluoromethyl)phenyl]methoxy]-2-methyl-azetidin-1-yl]-[(3R or 3S)-3-(1H-triazol-5-yl)pyrrolidin-1-yl]methanone

### Example 140

### (−)-[3-[[2-Fluoro-4-(trifluoromethyl)phenyl]methoxy]-2-methyl-azetidin-1-yl]-[(3R or 3S)-3-(1H-triazol-5-yl)pyrrolidin-1-yl]methanone and Example 141 (+)-[3-[[2-Fluoro-4-(trifluoromethyl)phenyl]methoxy]-2-methyl-azetidin-1-yl]-[(3R or 3S)-3-(1H-triazol-5-yl)pyrrolidin-1-yl]methanone

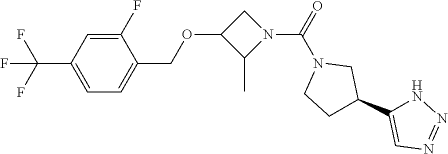 [SMILES: CC1C(OCc2ccc(C(F)(F)F)cc2F)CN1C(=O)N1CC[C@H](c2cnn[nH]2)C1]

To a solution of 3-[[2-fluoro-4-(trifluoromethyl)phenyl]methoxy]-2-methyl-azetidine 2,2,2-trifluoroacetic acid (200.0 mg, 0.530 mmol, CAS RN2411576-99-1) and DIPEA (1.0 mL, 2.12 mmol) in ACN (15 mL) was added (4-nitrophenyl) (3R or 3S)-3-(1H-triazol-5-yl)pyrrolidine-1-carboxylate (176.86 mg, 0.580 mmol, BB29) and the mixture was stirred at 80° C. for 12 h. The reaction mixture was poured into H₂O (5 mL), extracted with EtOAc (5 mL*3), the organic layer was evaporated. The crude product was purified with Prep-HPLC (0.225% v/vFA) and lyophilized to give the racemate. The racemate was separated using SFC Method IG to get the desired product 140 as a white solid (26.2 mg, 8%) MS (ESI): m/z=428.1 [M+H]⁺ and the desired product 141 as a white solid (17.2 mg, 4.9%) MS (ESI): m/z=428.1 [M+H]⁺ along with an impure mixture of products 140 and 141.

This impure mixture was separated using SFC Method IG to get the desired product 138 as a white solid (6.5 mg, 2%) MS (ESI): m/z=428.1 [M+H]⁺ and the desired product 139 as a white solid (5.6 mg, 1.7%) MS (ESI): m/z=428.1 [M+H]⁺.

### Example 138. Specific Rotation: +48.08°

### Example 139. Specific Rotation: −45.84°

### Example 140. Specific Rotation: −13.67°

### Example 141. Specific Rotation: +55.40°

### Example 182

### Sodium [3-[[2-fluoro-4-(trifluoromethyl)phenyl]methoxy]azetidin-1-yl]-[rac-(3S)-3-(1H-triazol-5-yl)pyrrolidin-1-yl]methanone

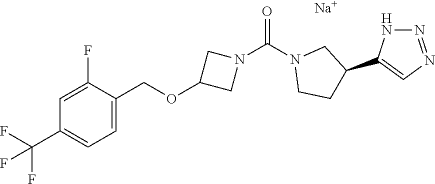 [SMILES: [Na+] ; O=C(N1CC(OCc2ccc(C(F)(F)F)cc2F)C1)N1CC[C@H](c2cnn[nH]2)C1]

To a solution of(S)-(3-(1H-1,2,3-triazol-5-yl)pyrrolidin-1-yl) (3-((2-fluoro-4-(trifluoromethyl)benzyl)oxy)azetidin-1-yl)methanone (50 mg, 121 μmol, example 36) in MeOH (0.2 mL) and THF (0.1 mL) was added sodium methoxide 30% in MeOH (22.5 μL, 121 μmol) and the clear and colorless solution was stirred at RT for 2.5 hours. TBME (2 mL) was added dropwise. This led to a precipitation. The suspension was stirred at RT for 4 hours, then filtered. The filter cake was washed with TBME and dried at high vacuum to get to get the desired compound as a colorless solid (0.035 g; 66.5%). MS (ESI): m/z=414.2 [M+H]⁺.

The following examples were prepared in analogy to examples 1 to 841 using building blocks which are either commercial or synthesized by person skilled in the art in analogy to the building blocks described in the this application from BB1 to BB119.

| | | | MS |
|---|---|---|---|
| Ex. | Systematic Name | Structure | m/z |
| 194 | [3-[5-(2- Chlorophenoxy)pyrazin-2- yl]azetidin-1-yl]-[(3S)-3-(1H- triazol-5-yl)pyrrolidin-1- yl]methanone | 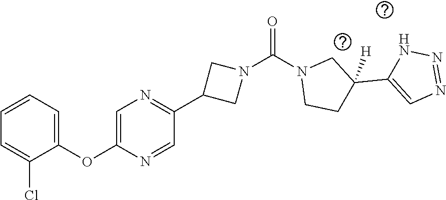 [SMILES: O=C(N1CC(c2cnc(Oc3ccccc3Cl)cn2)C1)N1CC[C@H](c2cnn[nH]2)C1] | 426.2 [M + H]⁺ |
| 197 | [(3S)-3-(1H-Triazol-5- yl)pyrrolidin-1-yl]-[3-[5-[1- (trifluoromethyl)cyclopropyl]- 1,2,4-oxadiazol-3-yl]azetidin-1- yl]methanone | 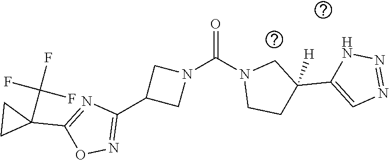 [SMILES: O=C(N1CC(c2noc(C3(C(F)(F)F)CC3)n2)C1)N1CC[C@H](c2cnn[nH]2)C1] | 398.2 [M + H]⁺ |
| 198 | [3-[6-(2,4-Dichlorophenoxy)-3- pyridyl]azetidin-1-yl]-[(3S)-3- (1H-triazol-5-yl)pyrrolidin-1- yl]methanone | 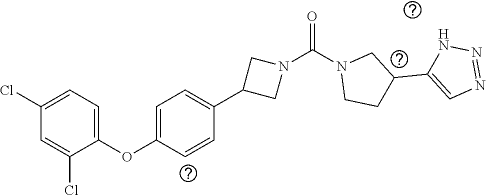 [SMILES: O=C(N1CC(c2ccc(Oc3ccc(Cl)cc3Cl)cc2)C1)N1CCC(c2cnn[nH]2)C1] | 459.2 [M + H]⁺ |
| 202 | [3-(2-Chloro-4-propylsulfonyl- phenyl)azetidin-1-yl]-[(3S)-3- (1H-triazol-5-yl)pyrrolidin-1- yl]methanone | 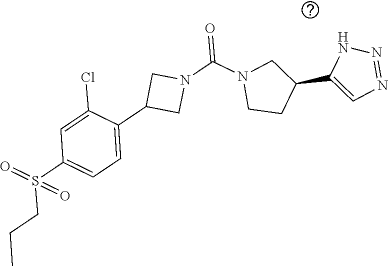 [SMILES: CCCS(=O)(=O)c1ccc(C2CN(C(=O)N3CC[C@H](c4cnn[nH]4)C3)C2)c(Cl)c1] | 438.3 [M + H]⁺ |
| 205 | 1-[3-[4-(2-Chloro-4- methylsulfonyl- phenyl)phenyl]azetidine-1- carbonyl]pyrrolidine-3- carboxamide | 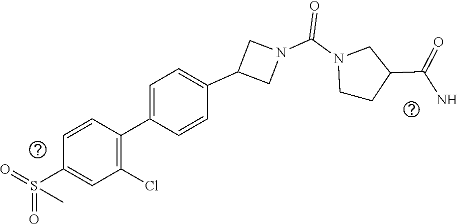 [SMILES: CS(=O)(=O)c1ccc(-c2ccc(C3CN(C(=O)N4CCC(C(N)=O)C4)C3)cc2)c(Cl)c1] | 462.3 [M + H]⁺ |
| 206 | [3-(5-tert-Butyl-2- pyridyl)azetidin-1-yl]-[(3S)-3- (1H-triazol-5-yl)pyrrolidin-1- yl]methanone | 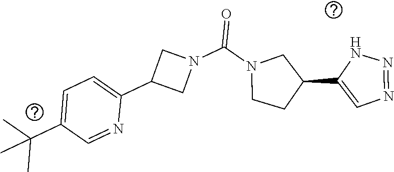 [SMILES: CC(C)(C)c1ccc(C2CN(C(=O)N3CC[C@H](c4cnn[nH]4)C3)C2)nc1] | 355.3 [M + H]⁺ |
| 207 | 1-[3-[4-(2-Chloro-4- methylsulfonyl- phenyl)phenyl]azetidine-1- carbonyl]pyrrolidine-3- carboxylic acid | 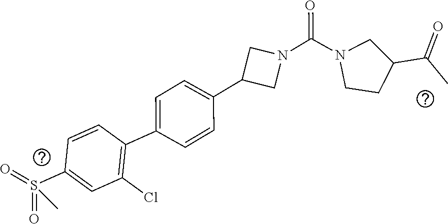 [SMILES: CC(=O)C1CCN(C(=O)N2CC(c3ccc(-c4ccc(S(C)(=O)=O)cc4Cl)cc3)C2)C1] | 463.2 [M + H]⁺ |
| 208 | 1-[3-[4-(2,4- Difluorophenyl)phenyl]azetidine- 1-carbonyl]pyrrolidine-3- carboxylic acid | 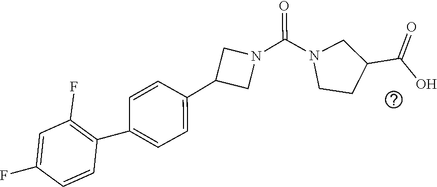 [SMILES: O=C(O)C1CCN(C(=O)N2CC(c3ccc(-c4ccc(F)cc4F)cc3)C2)C1] | 387.2 [M + H]⁺ |
| 209 | 2-[4-[1-[(3S)-3-(1H-Triazol-5- yl)pyrrolidine-1- carbonyl]azetidin-3- yl]phenyl]sulfonylacetonitrile | 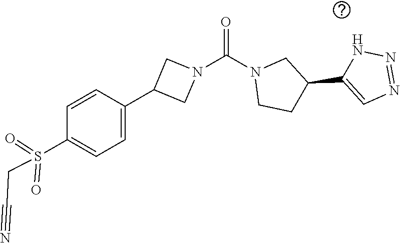 [SMILES: N#CCS(=O)(=O)c1ccc(C2CN(C(=O)N3CC[C@H](c4cnn[nH]4)C3)C2)cc1] | 401.2 [M + H]⁺ |
| 212 | [3-(4-Cyclohexyl- sulfonylphenyl)azetidin- 1-yl]-[(3S)-3-(1H-triazol-5- yl)pyrrolidin-1-yl]methanone | 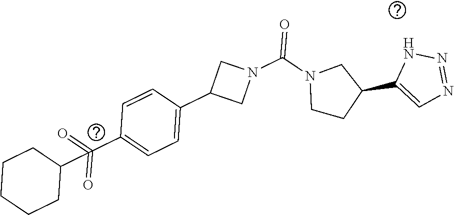 | 444.3 [M + H]⁺ |
| 213 | 1-[5-[1-[(3S)-3-(1H-Triazol-5- yl)pyrrolidine-1- carbonyl]azetidin-3-yl]-2- pyridyl]cyclobutanecarbonitrile | 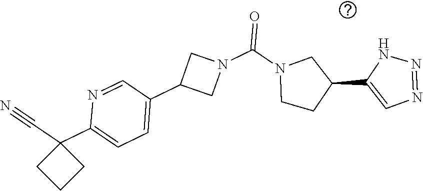 [SMILES: N#CC1(c2ccc(C3CN(C(=O)N4CC[C@H](c5cnn[nH]5)C4)C3)cn2)CCC1] | 378.2 [M + H]⁺ |
| 214 | [6-[(2,4- Difluorophenyl)methyl]-2,6- diazaspiro[3.3]heptan-2-yl]- [(3S)-3-(1H-triazol-5- yl)pyrrolidin-1-yl]methanone | 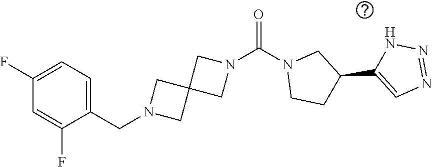 [SMILES: O=C(N1CC[C@H](c2cnn[nH]2)C1)N1CC2(CN(Cc3ccc(F)cc3F)C2)C1] | 389.3 [M + H]⁺ |
| 218 | [3-[4-(N- Phenylanilino)phenyl]azetidin-1- yl]-[(3S)-3-(1H-triazol-5- yl)pyrrolidin-1-yl]methanone | 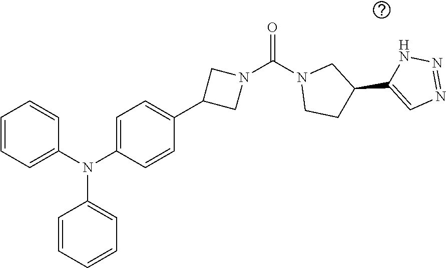 [SMILES: O=C(N1CC(c2ccc(N(c3ccccc3)c3ccccc3)cc2)C1)N1CC[C@H](c2cnn[nH]2)C1] | 465.3 [M + H]⁺ |
| 220 | [3-[6-[(5-Methoxy-2-pyridyl)- methyl-amino]-3- pyridyl]azetidin-1-yl]-[(3S)-3- (1H-triazol-5-yl)pyrrolidin-1- yl]methanone | 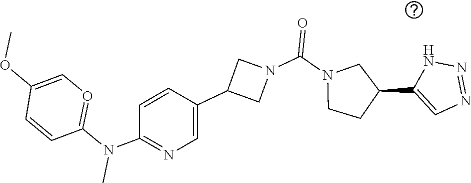 | 435.4 [M + H]⁺ |
| 221 | [3-[6-[(5-Ethyl-2-pyridyl)- methyl-amino]-3- pyridyl]azetidin-1-yl]-[(3S)-3- (1H-triazol-5-yl)pyrrolidin-1- yl]methanone | 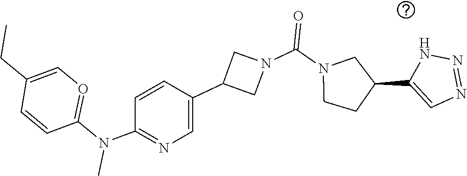 | 433.4 [M + H]⁺ |
| 222 | [3-[6-[(5-Cyclopropyl-2- pyridyl)-methyl-amino]-3- pyridyl]azetidin-1-yl]-[(3S)-3- (1H-triazol-5-yl)pyrrolidin-1- yl]methanone | 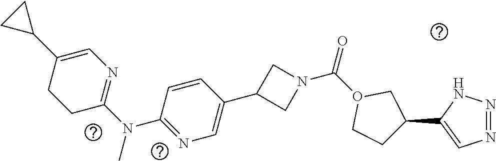 | 445.4 [M + H]⁺ |
| 223 | [3-[4-(N- Methylanilino)phenyl]azetidin- 1-yl]-[3-(1H-1,2,4-triazol-5- yl)pyrrolidin-1-yl]methanone | 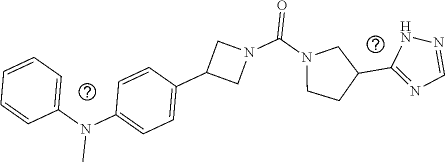 [SMILES: CN(c1ccccc1)c1ccc(C2CN(C(=O)N3CCC(c4ncn[nH]4)C3)C2)cc1] | 403.3 [M + H]⁺ |
| 224 | [3-[6-(N-Methylanilino)-3- pyridyl]azetidin-1-yl]-[(3S)-3- (1H-triazol-5-yl)pyrrolidin-1- yl]methanone | 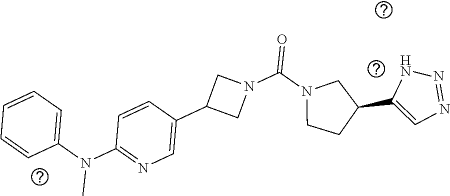 [SMILES: CN(c1ccccc1)c1ccc(C2CN(C(=O)N3CC[C@H](c4cnn[nH]4)C3)C2)cn1] | 404.3 [M + H]⁺ |
| 225 | [3-[6-(4-Isopropyl-N-methyl- anilino)-3-pyridyl]azetidin-1-yl]- [(3S)-3-(1H-triazol-5- yl)pyrrolidin-1-yl]methanone | 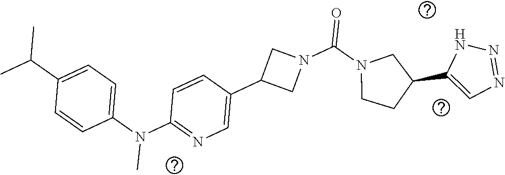 [SMILES: CC(C)c1ccc(N(C)c2ccc(C3CN(C(=O)N4CC[C@H](c5cnn[nH]5)C4)C3)cn2)cc1] | 446.3 [M + H]⁺ |
| 227 | [3-[6-[N- (Cyclopropylmethyl)anilino]-3- pyridyl]azetidin-1-yl]-[(3S)-3- (1H-triazol-5-yl)pyrrolidin-1- yl]methanone | 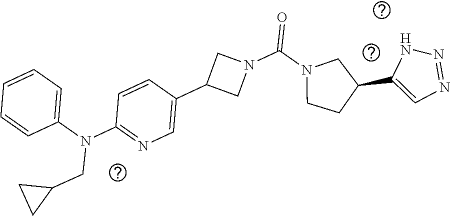 [SMILES: O=C(N1CC(c2ccc(N(CC3CC3)c3ccccc3)nc2)C1)N1CC[C@H](c2cnn[nH]2)C1] | 444.4 [M + H]⁺ |
| 230 | [3-[6-[N- (Cyclopropylmethyl)anilino]-3- pyridyl]azetidin-1-yl]-[3-(1H- tetrazol-5-yl)pyrrolidin-1- yl]methanone | 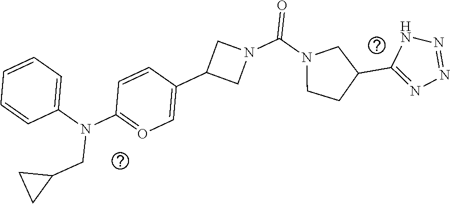 | 445.3 [M + H]⁺ |
| 232 | [2-(2-Methoxyphenyl)sulfonyl- 2,6-diazaspiro[3.3]heptan-6-yl]- [(3S)-3-(1H-triazol-5- yl)pyrrolidin-1-yl]methanone | 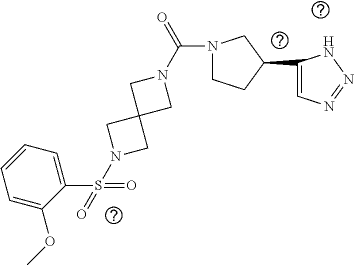 [SMILES: COc1ccccc1S(=O)(=O)N1CC2(CN(C(=O)N3CC[C@H](c4cnn[nH]4)C3)C2)C1] | 433.3 [M + H]⁺ |
| 234 | [2-(2,2- Dimethylpropylsulfonyl)-2,6- diazaspiro[3.3]heptan-6-yl]- [(3S)-3-(1H-triazol-5- yl)pyrrolidin-1-yl]methanone | 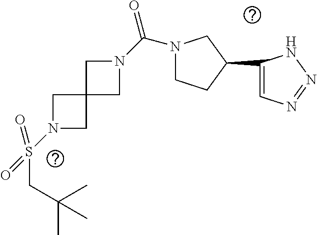 [SMILES: CC(C)(C)CS(=O)(=O)N1CC2(CN(C(=O)N3CC[C@H](c4cnn[nH]4)C3)C2)C1] | 397.3 [M + H]⁺ |
| 236 | [2-[4-Fluoro-2- (trifluoromethyl)phenyl]sulfonyl- 2,6-diazaspiro[3.3]heptan-6-yl]- [(3R)-3-(4H-1,2,4-triazol-3- yl)pyrrolidin-1-yl]methanone | 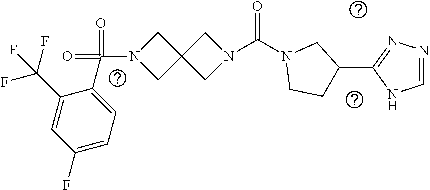 | 489.3 [M + H]⁺ |
| 237 | [2-[4-Fluoro-2- (trifluoromethyl)phenyl]sulfonyl- 2,6-diazaspiro[3.3]heptan-6-yl]- [(3S)-3-(4H-1,2,4-triazol-3- yl)pyrrolidin-1-yl]methanone | 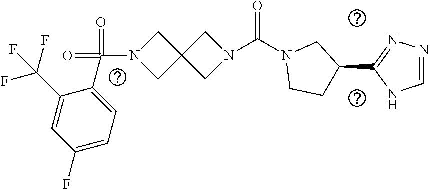 | 489.3 [M + H]⁺ |
| 238 | [3-[6-[N- (Cyclopropylmethyl)anilino]-3- pyridyl]azetidin-1-yl]-[(3S)-3- (1H-tetrazol-5-yl)pyrrolidin-1- yl]methanone | 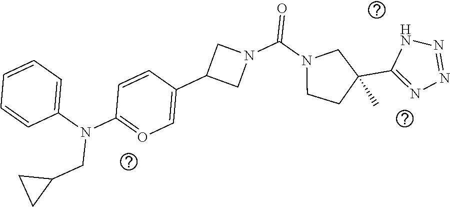 | 445.4 [M + H]⁺ |
| 239 | [3-[6-[N- (Cyclopropylmethyl)anilino]-3- pyridyl]azetidin-1-yl]-[(3R)-3- (1H-tetrazol-5-yl)pyrrolidin-1- yl]methanone | 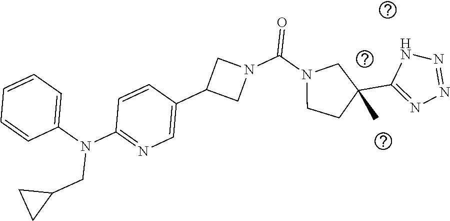 | 445.4 [M + H]⁺ |
| 240 | [3-[4-(4- Fluorophenoxy)phenyl]azetidin- 1-yl]-[3-(1H-tetrazol-5- yl)pyrrolidin-1-yl]methanone | 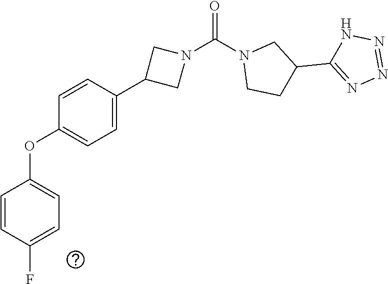 [SMILES: O=C(N1CC(c2ccc(Oc3ccc(F)cc3)cc2)C1)N1CCC(c2nnn[nH]2)C1] | 409.3 [M + H]⁺ |
| 241 | 1-[3-[6-[N- (Cyclopropylmethyl)anilino]-3- pyridyl]azetidine-1- carbonyl]pyrrolidine-3- carboxamide | 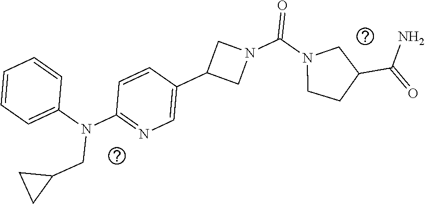 [SMILES: NC(=O)C1CCN(C(=O)N2CC(c3ccc(N(CC4CC4)c4ccccc4)nc3)C2)C1] | 420.4 [M + H]⁺ |
| 242 | [3-[4-(4- Fluorophenoxy)phenyl]azetidin- 1-yl]-[(3R)-3-(1H-tetrazol-5- yl)pyrrolidin-1-yl]methanone | 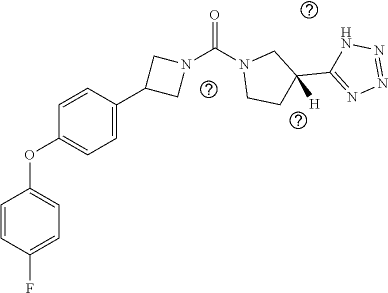 [SMILES: O=C(N1CC(c2ccc(Oc3ccc(F)cc3)cc2)C1)N1CC[C@@H](c2nnn[nH]2)C1] | 409.3 [M + H]⁺ |
| 243 | [3-[4-(4- Fluorophenoxy)phenyl]azetidin- 1-yl]-[(3S)-3-(1H-tetrazol-5- yl)pyrrolidin-1-yl]methanone | 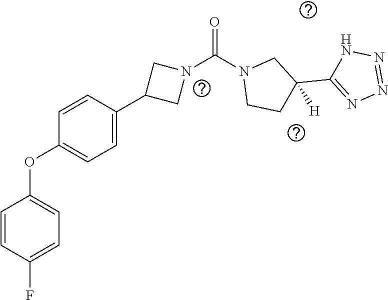 [SMILES: O=C(N1CC(c2ccc(Oc3ccc(F)cc3)cc2)C1)N1CC[C@H](c2nnn[nH]2)C1] | 409.3 [M + H]⁺ |
| 244 | [(3S)-3-(1,2,4-Triazol-4- yl)pyrrolidin-1-yl]-[3-[4-[4- (trifluoromethyl)pyrimidin-2- yl]oxyphenyl]azetidin-1- yl]methanone | 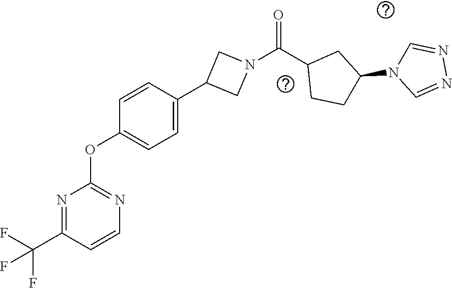 [SMILES: O=C(C1CC[C@H](n2cnnc2)C1)N1CC(c2ccc(Oc3nccc(C(F)(F)F)n3)cc2)C1] | 460.4 [M + H]⁺ |
| 245 | [3-[4-(4-Chloro-2- methylsulfonyl- phenyl)phenyl]azetidin-1-yl]-[3- (1H-tetrazol-5-yl)pyrrolidin-1- yl]methanone | 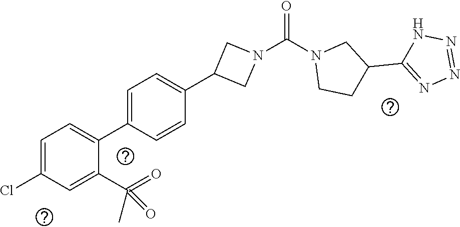 | 487.2 [M + H]⁺ |
| 246 | [(3S)-3-(1,2,4-Triazol-4- yl)pyrrolidin-1-yl]-[6-[6- (trifluoromethyl)pyrazin-2- yl]oxy-2-azaspiro[3.3]heptan-2- yl]methanone | 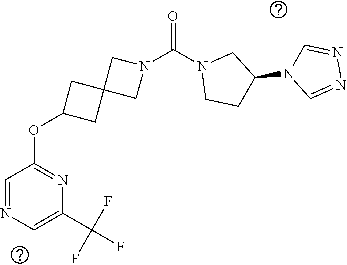 [SMILES: O=C(N1CC[C@H](n2cnnc2)C1)N1CC2(CC(Oc3cncc(C(F)(F)F)n3)C2)C1] | 424.5 [M + H]⁺ |
| 247 | [(3S)-3-(1,2,4-Triazol-4- yl)pyrrolidin-1-yl]-[6-[[1- (trifluoromethyl)cyclopropyl] methoxy]-2- azaspiro[3.3]heptan-2- yl]methanone | 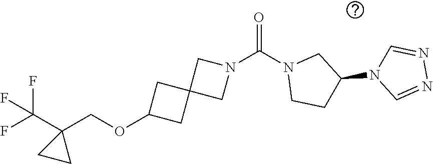 [SMILES: O=C(N1CC[C@H](n2cnnc2)C1)N1CC2(CC(OCC3(C(F)(F)F)CC3)C2)C1] | 400.4 [M + H]⁺ |
| 248 | (3S)-1-[3-[6-[N- (Cyclopropylmethyl)anilino]-3- pyridyl]azetidine-1- carbonyl]pyrrolidine-3- carboxamide | 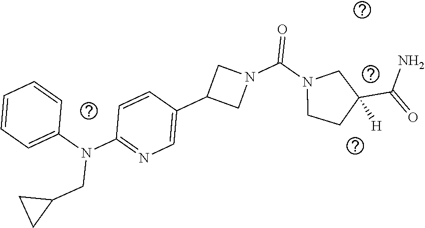 [SMILES: NC(=O)[C@H]1CCN(C(=O)N2CC(c3ccc(N(CC4CC4)c4ccccc4)nc3)C2)C1] | 420.3 [M + H]⁺ |
| 249 | (3R)-1-[3-[6-[N- (Cyclopropylmethyl)anilino]-3- pyridyl]azetidine-1- carbonyl]pyrrolidine-3- carboxamide | 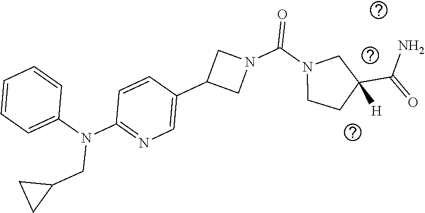 [SMILES: NC(=O)[C@@H]1CCN(C(=O)N2CC(c3ccc(N(CC4CC4)c4ccccc4)nc3)C2)C1] | 420.3 [M + H]⁺ |
| 250 | [6-[[4-Fluoro-2- (trifluoromethyl)phenyl]methyl]- 2,6-diazaspiro[3.3]heptan-2-yl]- [(3S)-3-(4H-1,2,4-triazol-3- yl)pyrrolidin-1-yl]methanone | 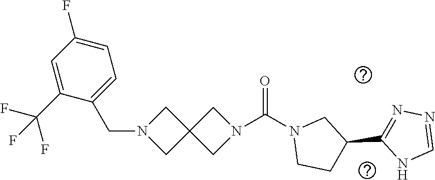 [SMILES: O=C(N1CC[C@H](c2nnc[nH]2)C1)N1CC2(CN(Cc3ccc(F)cc3C(F)(F)F)C2)C1] | 439.3 [M + H]⁺ |
| 251 | [(3S)-3-(4H-1,2,4-Triazol-3- yl)pyrrolidin-1-yl]-[3-[4-[3- (trifluoromethyl)azetidin-1- yl]phenyl]azetidin-1- yl]methanone | 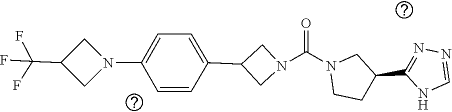 [SMILES: O=C(N1CC(c2ccc(N3CC(C(F)(F)F)C3)cc2)C1)N1CC[C@H](c2nnc[nH]2)C1] | 421.3 [M + H]⁺ |
| 252 | 1-[3-[4-(Benzene- sulfonyl)phenyl]azetidine- 1-carbonyl]pyrrolidine-3- carboxamide | 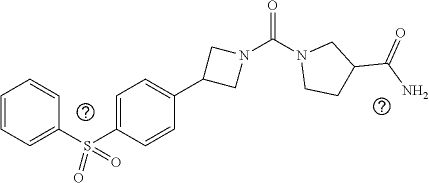 [SMILES: NC(=O)C1CCN(C(=O)N2CC(c3ccc(S(=O)(=O)c4ccccc4)cc3)C2)C1] | 414.3 [M + H]⁺ |
| 253 | [2-[[2-Fluoro-4- (trifluoromethyl)phenyl]methyl]- 2,7-diazaspiro[3.5]nonan-7-yl]- [3-(4H-1,2,4-triazol-3- yl)pyrrolidin-1-yl]methanone | 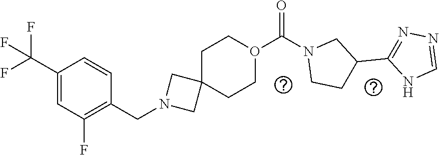 | 467.3 [M + H]⁺ |
| 255 | [2-[(2-Chloro-4-fluoro- phenyl)methyl]-2,7- diazaspiro[3.5]nonan-7-yl]-[3- (4H-1,2,4-triazol-3- yl)pyrrolidin-1-yl]methanone | 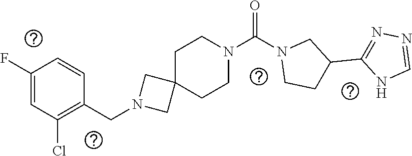 [SMILES: O=C(N1CCC2(CC1)CN(Cc1ccc(F)cc1Cl)C2)N1CCC(c2nnc[nH]2)C1] | 433.4 [M + H]⁺ |
| 256 | [3-[(2-Fluoro-4-methylsulfonyl- phenyl)methoxy]azetidin-1-yl]- [3-(4H-1,2,4-triazol-3- yl)pyrrolidin-1-yl]methanone | 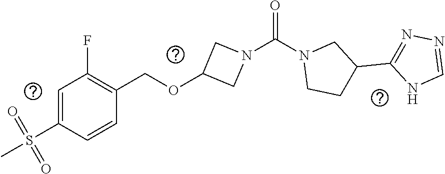 [SMILES: CS(=O)(=O)c1ccc(COC2CN(C(=O)N3CCC(c4nnc[nH]4)C3)C2)c(F)c1] | 424.4 [M + H]⁺ |
| 257 | 1-[3-[4-[1- (Trifluoromethyl)cyclopropyl] phenyl]azetidine-1- carbonyl]pyrrolidine-3- carboxamide | 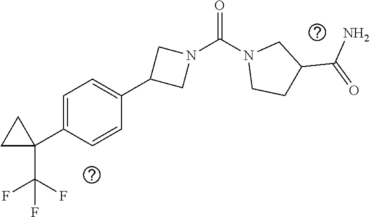 [SMILES: NC(=O)C1CCN(C(=O)N2CC(c3ccc(C4(C(F)(F)F)CC4)cc3)C2)C1] | 382.2 [M + H]⁺ |
| 258 | 1-[3-[4-[3- (Trifluoromethyl)azetidin-1- yl]phenyl]azetidine-1- carbonyl]pyrrolidine-3- carboxamide | 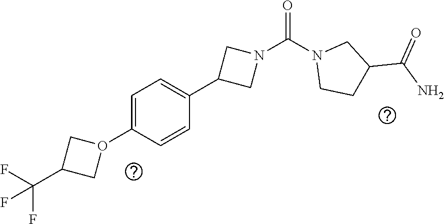 | 397.2 [M + H]⁺ |
| 259 | [3-[4-(4-Chloro-2- methylsulfonyl- phenyl)phenyl]azetidin-1-yl]- [(3R)-3-(1H-tetrazol-5- yl)pyrrolidin-1-yl]methanone | 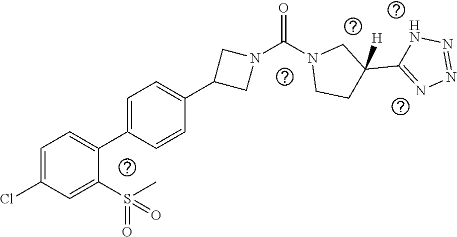 [SMILES: CS(=O)(=O)c1cc(Cl)ccc1-c1ccc(C2CN(C(=O)N3CC[C@@H](c4nnn[nH]4)C3)C2)cc1] | 487.2 [M + H]⁺ |
| 260 | [3-[4-(4-Chloro-2- methylsulfonyl- phenyl)phenyl]azetidin-1-yl]- [(3S)-3-(1H-tetrazol-5- yl)pyrrolidin-1-yl]methanone | 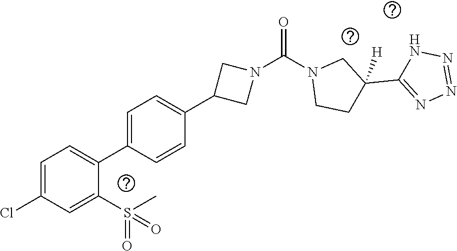 [SMILES: CS(=O)(=O)c1cc(Cl)ccc1-c1ccc(C2CN(C(=O)N3CC[C@H](c4nnn[nH]4)C3)C2)cc1] | 487.2 [M + H]⁺ |
| 262 | [6-[[2-Fluoro-4- (trifluoromethyl)phenyl]methyl]- 2,6-diazaspiro[3.3]heptan-2-yl]- [3-(4H-1,2,4-triazol-3- yl)pyrrolidin-1-yl]methanone | 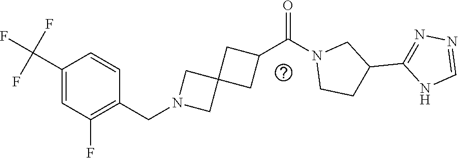 [SMILES: O=C(C1CC2(C1)CN(Cc1ccc(C(F)(F)F)cc1F)C2)N1CCC(c2nnc[nH]2)C1] | 439.3 [M + H]⁺ |
| 263 | [3-(4H-1,2,4-Triazol-3- yl)pyrrolidin-1-yl]-[3-[[6- (trifluoromethyl)-3- pyridyl]methoxy]azetidin-1- yl]methanone | 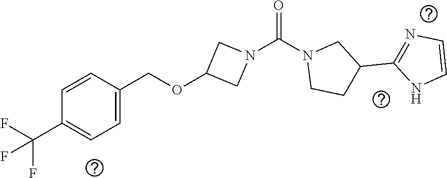 [SMILES: O=C(N1CC(OCc2ccc(C(F)(F)F)cc2)C1)N1CCC(c2ncc[nH]2)C1] | 397.4 [M + H]⁺ |
| 264 | [3-(4H-1,2,4-Triazol-3- yl)pyrrolidin-1-yl]-[3-[[5- (trifluoromethyl)-3- pyridyl]methoxy]azetidin-1- yl]methanone | 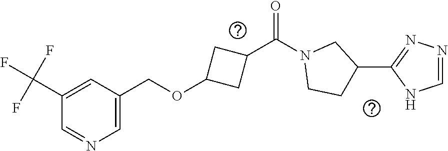 [SMILES: O=C(C1CC(OCc2cncc(C(F)(F)F)c2)C1)N1CCC(c2nnc[nH]2)C1] | 397.4 [M + H]⁺ |
| 265 | [3-(4H-1,2,4-Triazol-3- yl)pyrrolidin-1-yl]-[6-[5- (trifluoromethyl)-3-pyridyl]oxy]- 2-azaspiro[3.3]heptan-2- yl]methanone | 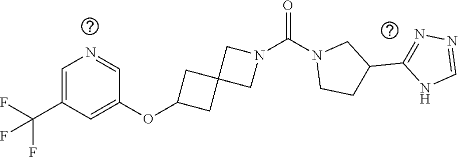 [SMILES: O=C(N1CCC(c2nnc[nH]2)C1)N1CC2(CC(Oc3cncc(C(F)(F)F)c3)C2)C1] | 423.2 [M + H]⁺ |
| 266 | [6-[(5-Chloro-3-pyridyl)oxy]-2- azaspiro[3.3]heptan-2-yl]-[3- (4H-1,2,4-triazol-3- yl)pyrrolidin-1-yl]methanone | 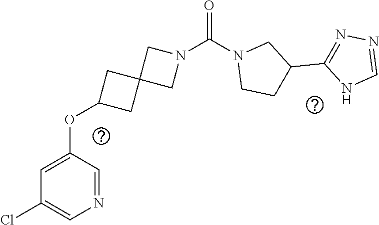 [SMILES: O=C(N1CCC(c2nnc[nH]2)C1)N1CC2(CC(Oc3cncc(Cl)c3)C2)C1] | 389.2 [M + H]⁺ |
| 267 | [6-[(6-Chloro-3-pyridyl)oxy]-2- azaspiro[3.3]heptan-2-yl]-[3- (4H-1,2,4-triazol-3- yl)pyrrolidin-1-yl]methanone | 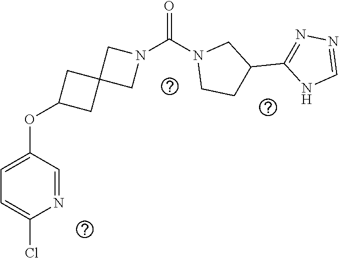 [SMILES: O=C(N1CCC(c2nnc[nH]2)C1)N1CC2(CC(Oc3ccc(Cl)nc3)C2)C1] | 389.2 [M + H]⁺ |
| 270 | (3S)-1-[3-[4-(Benzene- sulfonyl)phenyl]azetidine- 1-carbonyl]pyrrolidine-3- carboxamide | 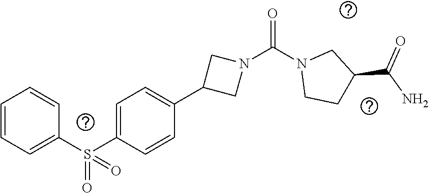 [SMILES: NC(=O)[C@H]1CCN(C(=O)N2CC(c3ccc(S(=O)(=O)c4ccccc4)cc3)C2)C1] | 414.2 [M + H]⁺ |
| 271 | (3R)-1-[3-[4-(Benzene- sulfonyl)phenyl]azetidine- 1-carbonyl]pyrrolidine-3- carboxamide | 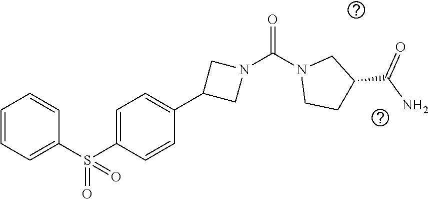 [SMILES: NC(=O)[C@@H]1CCN(C(=O)N2CC(c3ccc(S(=O)(=O)c4ccccc4)cc3)C2)C1] | 414.2 [M + H]⁺ |
| 272 | [3-[(2-Fluoro-4-methylsulfonyl- phenyl)methoxy]azetidin-1-yl]- [(3S)-3-(1H-triazol-5- yl)pyrrolidin-1-yl]methanone | 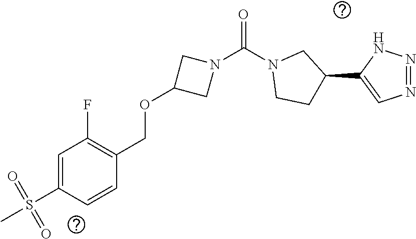 [SMILES: CS(=O)(=O)c1ccc(COC2CN(C(=O)N3CC[C@H](c4cnn[nH]4)C3)C2)c(F)c1] | 424.4 [M + H]⁺ |
| 273 | 2-Cyclopropyl-6-[1-[(3S)-3- (1,2,4-triazol-4-yl)pyrrolidine-1- carbonyl]azetidin-3-yl]oxy- benzonitrile | 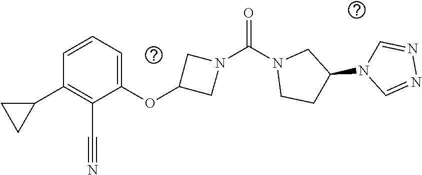 [SMILES: N#Cc1c(OC2CN(C(=O)N3CC[C@H](n4cnnc4)C3)C2)cccc1C1CC1] | 379.3 [M + H]⁺ |
| 275 | (3S)-1-[2-[2- (Trifluoromethyl)phenyl]sulfonyl- 2,6-diazaspiro[3.3]heptane-6- carbonyl]pyrrolidine-3- carboxamide | 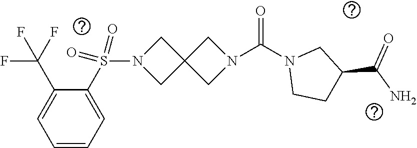 [SMILES: NC(=O)[C@H]1CCN(C(=O)N2CC3(C2)CN(S(=O)(=O)c2ccccc2C(F)(F)F)C3)C1] | 447.3 [M + H]⁺ |
| 276 | (3S)-1-[3-[[[3- (Trifluoromethyl)phenyl]sulfonyl- amino]methyl]azetidine-1- carbonyl]pyrrolidine-3- carboxamide | 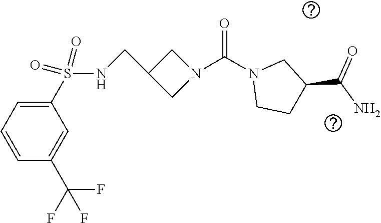 [SMILES: NC(=O)[C@H]1CCN(C(=O)N2CC(CNS(=O)(=O)c3cccc(C(F)(F)F)c3)C2)C1] | 435.3 [M + H]⁺ |
| 277 | (3S)-1-[3-[[[4- (Trifluoromethyl)phenyl]sulfonyl- amino]methyl]azetidine-1- carbonyl]pyrrolidine-3- carboxamide | 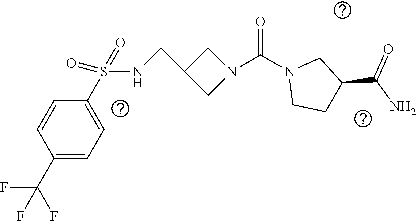 [SMILES: NC(=O)[C@H]1CCN(C(=O)N2CC(CNS(=O)(=O)c3ccc(C(F)(F)F)cc3)C2)C1] | 435.3 [M + H]⁺ |
| 278 | (3S)-1-[6-[[1- (Trifluoromethyl)cyclopropyl] methoxy]-2- azaspiro[3.3]heptane- 2-carbonyl]pyrrolidine-3- carboxamide | 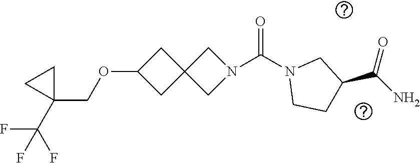 [SMILES: NC(=O)[C@H]1CCN(C(=O)N2CC3(CC(OCC4(C(F)(F)F)CC4)C3)C2)C1] | 376.3 [M + H]⁺ |
| 279 | (3S)-1-[6-[[6-(Trifluoromethyl)- 3-pyridyl]oxy]-2- azaspiro[3.3]heptane-2- carbonyl]pyrrolidine-3- carboxamide | 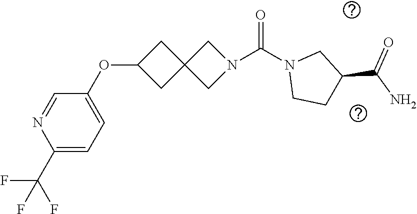 [SMILES: NC(=O)[C@H]1CCN(C(=O)N2CC3(CC(Oc4ccc(C(F)(F)F)nc4)C3)C2)C1] | 399.3 [M + H]⁺ |
| 280 | (3S)-1-[6-[[5-(Trifluoromethyl)- 2-pyridyl]methyl]-2,6- diazaspiro[3.3]heptane-2- carbonyl]pyrrolidine-3- carboxamide | 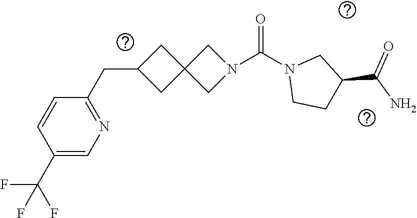 [SMILES: NC(=O)[C@H]1CCN(C(=O)N2CC3(CC(Cc4ccc(C(F)(F)F)cn4)C3)C2)C1] | 398.3 [M + H]⁺ |
| 281 | (3S)-1-[3-[[6-(Trifluoromethyl)- 3-pyridyl]methoxy]azetidine-1- carbonyl]pyrrolidine-3- carboxamide | 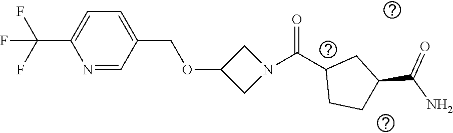 [SMILES: NC(=O)[C@H]1CCC(C(=O)N2CC(OCc3ccc(C(F)(F)F)nc3)C2)C1] | 373.2 [M + H]⁺ |
| 282 | (3S)-1-[3-[[5-(Trifluoromethyl)- 3-pyridyl]methoxy]azetidine-1- carbonyl]pyrrolidine-3- carboxamide | 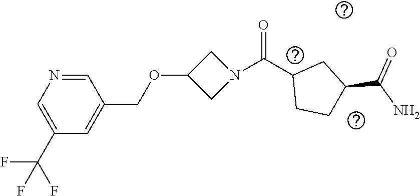 [SMILES: NC(=O)[C@H]1CCC(C(=O)N2CC(OCc3cncc(C(F)(F)F)c3)C2)C1] | 373.2 [M + H]⁺ |
| 283 | (3S)-1-[6-[[[1- (Trifluoromethyl)cyclopropyl] amino]methyl]-2- azaspiro[3.3]heptane-2- carbonyl]pyrrolidine-3- carboxamide | 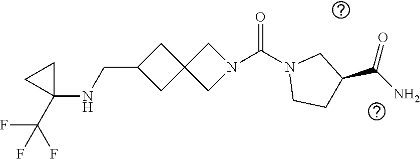 [SMILES: NC(=O)[C@H]1CCN(C(=O)N2CC3(CC(CNC4(C(F)(F)F)CC4)C3)C2)C1] | 375.3 [M + H]⁺ |
| 284 | [3-[4-(3-Chloropyridazin-4- yl)oxyphenyl]azetidin-1-yl]- [(3R)-3-(tetrazol-1-yl)pyrrolidin- 1-yl]methanone | 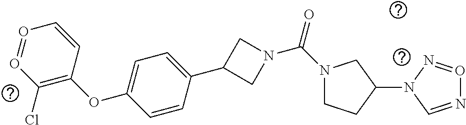 | 427.2 [M + H]⁺ |
| 285 | [3-[[2-Fluoro-4- (trifluoromethyl)phenyl]methyl- amino]azetidin-1-yl]-[(3S)-3- (1H-triazol-5-yl)pyrrolidin-1- yl]methanone | 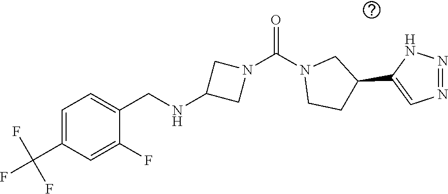 [SMILES: O=C(N1CC(NCc2ccc(C(F)(F)F)cc2F)C1)N1CC[C@H](c2cnn[nH]2)C1] | 413.4 [M + H]⁺ |
| 287 | [3-[(2-Fluoro-4-methylsulfonyl- phenyl)methoxy]azetidin-1-yl]- [(3S)-3-(1,2,4-triazol-4- yl)pyrrolidin-1-yl]methanone | 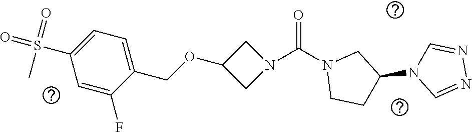 [SMILES: CS(=O)(=O)c1ccc(COC2CN(C(=O)N3CC[C@H](n4cnnc4)C3)C2)c(F)c1] | 424.4 [M + H]⁺ |
| 288 | [(3S)-3-(1,2,4-Triazol-4- yl)pyrrolidin-1-yl]-[2-[2- (trifluoromethyl)phenyl]sulfonyl- 2,6-diazaspiro[3.3]heptan-6- yl]methanone | 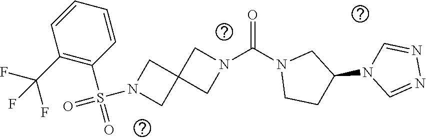 [SMILES: O=C(N1CC[C@H](n2cnnc2)C1)N1CC2(C1)CN(S(=O)(=O)c1ccccc1C(F)(F)F)C2] | 471.3 [M + H]⁺ |
| 289 | 1-[4-[1-[(3S)-3-(1,2,4-Triazol-4- yl)pyrrolidine-1- carbonyl]azetidin-3- yl]phenyl]cyclopropane- carbonitrile | 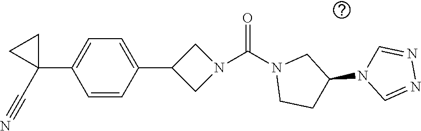 [SMILES: N#CC1(c2ccc(C3CN(C(=O)N4CC[C@H](n5cnnc5)C4)C3)cc2)CC1] | 363.3 [M + H]⁺ |
| 290 | [3-[[2-Fluoro-4- (trifluoromethyl)phenyl]methyl- amino]azetidin-1-yl]-[(3S)-3- (1,2,4-triazol-4-yl)pyrrolidin-1- yl]methanone | 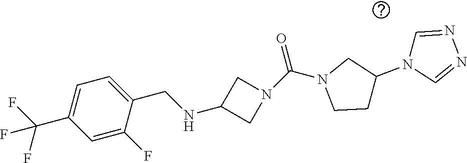 [SMILES: O=C(N1CC(NCc2ccc(C(F)(F)F)cc2F)C1)N1CCC(n2cnnc2)C1] | 413.4 [M + H]⁺ |
| 291 | N-[2-[(3S)-3-(1,2,4-Triazol-4- yl)pyrrolidine-1-carbonyl]-2- azaspiro[3.3]heptan-6-yl]-1- (trifluoromethyl)cyclopropane- carboxamide | 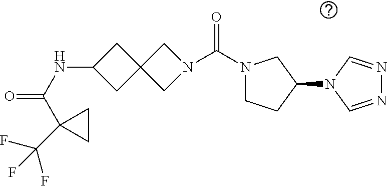 [SMILES: O=C(N1CC[C@H](n2cnnc2)C1)N1CC2(CC(NC(=O)C3(C(F)(F)F)CC3)C2)C1] | 413.4 [M + H]⁺ |
| 293 | (3S)-1-[3-[2-[1- (Trifluoromethyl)cyclopropyl] pyrimidin-5-yl]azetidine-1- carbonyl]pyrrolidine-3- carboxamide | 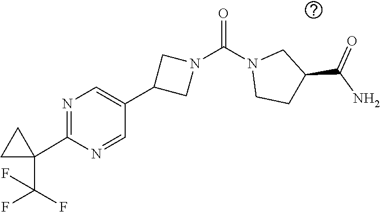 [SMILES: NC(=O)[C@H]1CCN(C(=O)N2CC(c3cnc(C4(C(F)(F)F)CC4)nc3)C2)C1] | 384.2 [M + H]⁺ |
| 294 | (3S)-1-[3-[6-[1- (Trifluoromethyl)cyclopropyl]- 3-pyridyl]azetidine-1- carbonyl]pyrrolidine-3- carboxamide | 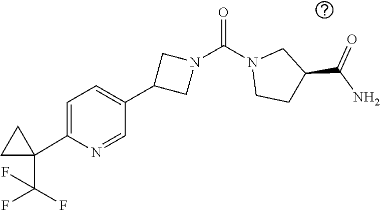 [SMILES: NC(=O)[C@H]1CCN(C(=O)N2CC(c3ccc(C4(C(F)(F)F)CC4)nc3)C2)C1] | 383.2 [M + H]⁺ |
| 295 | [3-(4H-1,2,4-Triazol-3- yl)pyrrolidin-1-yl]-[3-[[2- (trifluoromethyl)pyrimidin-5- yl]methoxy]azetidin-1- yl]methanone | 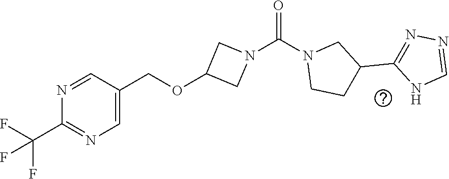 [SMILES: O=C(N1CC(OCc2cnc(C(F)(F)F)nc2)C1)N1CCC(c2nnc[nH]2)C1] | 398.2 [M + H]⁺ |
| 297 | 1-[3-[4-(4-Chloro-2- methylsulfonyl- phenyl)phenyl]azetidine-1- carbonyl]-N-ethyl-pyrrolidine-3- carboxamide | 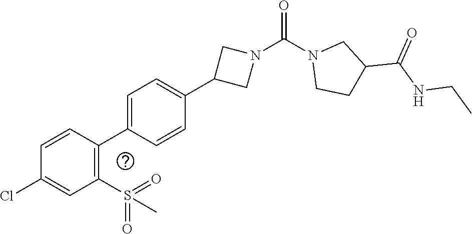 [SMILES: CCNC(=O)C1CCN(C(=O)N2CC(c3ccc(-c4ccc(Cl)cc4S(C)(=O)=O)cc3)C2)C1] | 490.3 [M + H]⁺ |
| 300 | (3S)-1-[3-[(2-Fluoro-4- methylsulfonyl- phenyl)methoxy]azetidine-1- carbonyl]pyrrolidine-3- carboxamide | 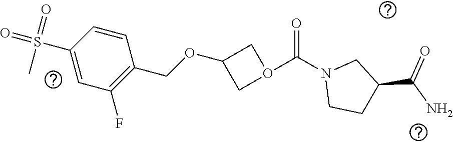 | 400.2 [M + H]⁺ |
| 301 | (3S)-1-[2-(2,4- Difluorophenyl)sulfonyl-2,6- diazaspiro[3.3]heptane-6- carbonyl]pyrrolidine-3- carboxamide | 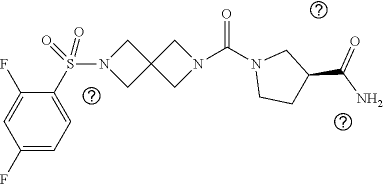 [SMILES: NC(=O)[C@H]1CCN(C(=O)N2CC3(C2)CN(S(=O)(=O)c2ccc(F)cc2F)C3)C1] | 415.2 [M + H]⁺ |
| 302 | (3S)-1-[2-[3-(Trifluoro- methyl)phenyl]sulfonyl- 2,6-diazaspiro[3.3]heptane-6- carbonyl]pyrrolidine-3- carboxamide | 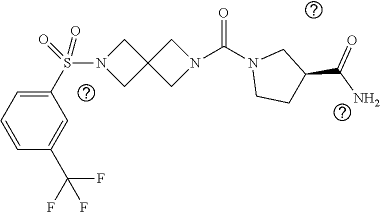 [SMILES: NC(=O)[C@H]1CCN(C(=O)N2CC3(C2)CN(S(=O)(=O)c2cccc(C(F)(F)F)c2)C3)C1] | 447.2 [M + H]⁺ |
| 303 | (3S)-1-[2-[3-(Trifluoro- methoxy)phenyl]sulfonyl- 2,6-diazaspiro[3.3]heptane- 6-carbonyl]pyrrolidine-3- carboxamide | 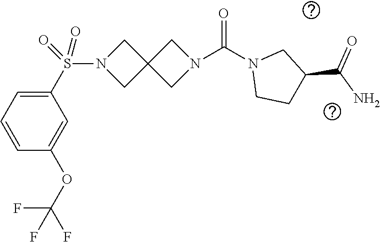 [SMILES: NC(=O)[C@H]1CCN(C(=O)N2CC3(C2)CN(S(=O)(=O)c2cccc(OC(F)(F)F)c2)C3)C1] | 463.2 [M + H]⁺ |
| 304 | rac-(3S)-1-[3-[5-[3- (Trifluoromethyl)pyrrolidin-1- yl]-2-pyridyl]azetidine-1- carbonyl]pyrrolidine-3- carboxamide | 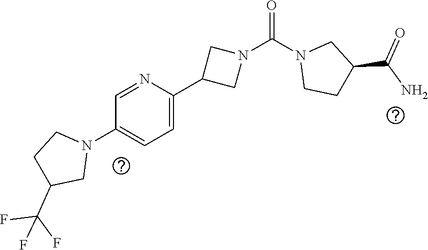 [SMILES: NC(=O)[C@H]1CCN(C(=O)N2CC(c3ccc(N4CCC(C(F)(F)F)C4)cn3)C2)C1] | 412.2 [M + H]⁺ |
| 307 | (3S)-1-[3-[4-(4-Chloro-2- methylsulfonyl- phenyl)phenyl]azetidine-1- carbonyl]-N-ethyl-pyrrolidine-3- carboxamide | 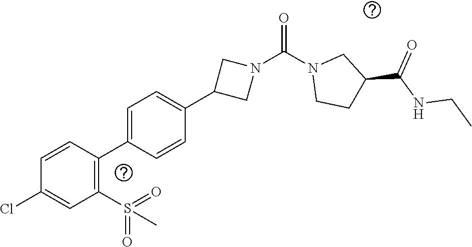 [SMILES: CCNC(=O)[C@H]1CCN(C(=O)N2CC(c3ccc(-c4ccc(Cl)cc4S(C)(=O)=O)cc3)C2)C1] | 490.2 [M + H]⁺ |
| 308 | (3R)-1-[3-[4-(4-Chloro-2- methylsulfonyl- phenyl)phenyl]azetidine-1- carbonyl]-N-ethyl-pyrrolidine-3- carboxamide | 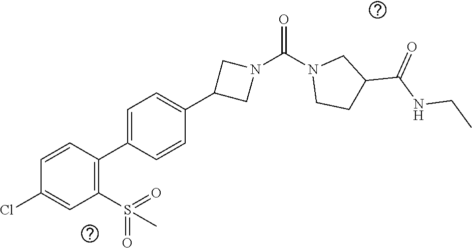 [SMILES: CCNC(=O)C1CCN(C(=O)N2CC(c3ccc(-c4ccc(Cl)cc4S(C)(=O)=O)cc3)C2)C1] | 490.2 [M + H]⁺ |
| 309 | [3-[6-(2-Chlorophenoxy)-3- pyridyl]azetidin-1-yl]-[(3R)-3- (tetrazol-1-yl)pyrrolidin-1- yl]methanone | 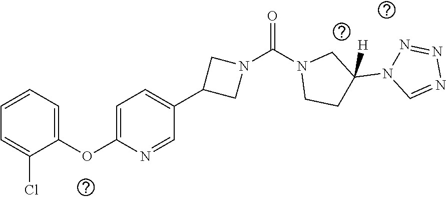 [SMILES: O=C(N1CC(c2ccc(Oc3ccccc3Cl)nc2)C1)N1CC[C@@H](n2cnnn2)C1] | 426.2 [M + H]⁺ |
| 310 | [3-[4-(4- Fluorophenoxy)phenyl]azetidin- 1-yl]-[(3S)-3-(tetrazol-1- yl)pyrrolidin-1-yl]methanone | 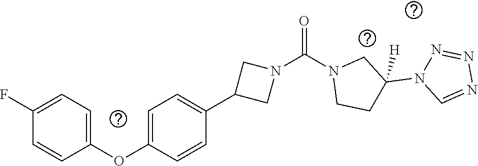 [SMILES: O=C(N1CC(c2ccc(Oc3ccc(F)cc3)cc2)C1)N1CC[C@H](n2cnnn2)C1] | 409.2 [M + H]⁺ |
| 311 | [3-[5-(2- Chlorophenoxy)pyrazin-2- yl]azetidin-1-yl]-[(3S)-3- (tetrazol-1-yl)pyrrolidin-1- yl]methanone | 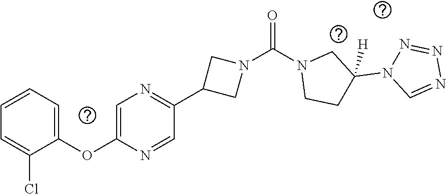 [SMILES: O=C(N1CC(c2cnc(Oc3ccccc3Cl)cn2)C1)N1CC[C@H](n2cnnn2)C1] | 427.2 [M + H]⁺ |
| 312 | [(3R)-3-(Tetrazol-1- yl)pyrrolidin-1-yl]-[6-[6- (trifluoromethyl)pyrazin-2- yl]oxy-2-azaspiro[3.3]heptan-2- yl]methanone | 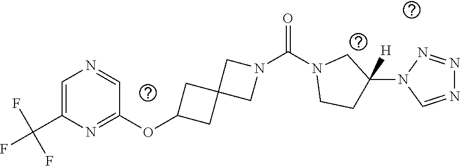 [SMILES: O=C(N1CC[C@@H](n2cnnn2)C1)N1CC2(CC(Oc3cncc(C(F)(F)F)n3)C2)C1] | 425.2 [M + H]⁺ |
| 313 | 2-Cyclopropyl-6-[1-[(3R)-3- (tetrazol-1-yl)pyrrolidine-1- carbonyl]azetidin-3-yl]oxy- benzonitrile | 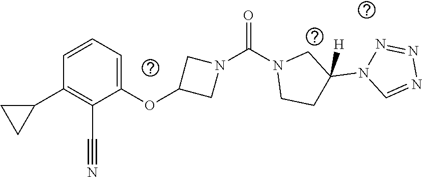 [SMILES: N#Cc1c(OC2CN(C(=O)N3CC[C@@H](n4cnnn4)C3)C2)cccc1C1CC1] | 380.4 [M + H]⁺ |
| 314 | [3-[5-(2- Chlorophenoxy)pyrazin-2- yl]azetidin-1-yl]-[(3R)-3- (tetrazol-1-yl)pyrrolidin-1- yl]methanone | 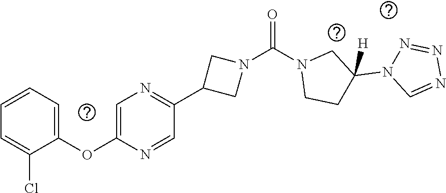 [SMILES: O=C(N1CC(c2cnc(Oc3ccccc3Cl)cn2)C1)N1CC[C@@H](n2cnnn2)C1] | 427.2 [M + H]⁺ |
| 315 | [(3S)-3-(Tetrazol-1- yl)pyrrolidin-1-yl]-[6-[6- (trifluoromethyl)pyrazin-2- yl]oxy-2-azaspiro[3.3]heptan-2- yl]methanone | 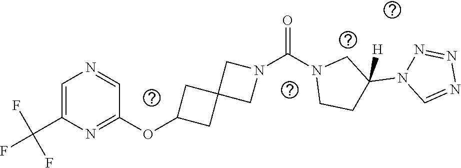 [SMILES: O=C(N1CC[C@@H](n2cnnn2)C1)N1CC2(CC(Oc3cncc(C(F)(F)F)n3)C2)C1] | 425.2 [M + H]⁺ |
| 317 | [3-[4-(4- Fluorophenoxy)phenyl]azetidin- 1-yl]-[(3R)-3-(tetrazol-1- yl)pyrrolidin-1-yl]methanone | 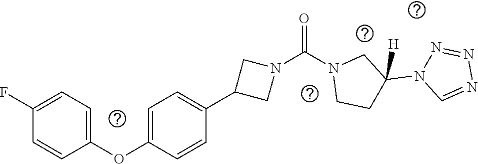 [SMILES: O=C(N1CC(c2ccc(Oc3ccc(F)cc3)cc2)C1)N1CC[C@@H](n2cnnn2)C1] | 409.2 [M + H]⁺ |
| 319 | 2-Cyclopropyl-6-[1-[(3S)-3- (tetrazol-1-yl)pyrrolidine-1- carbonyl]azetidin-3-yl]oxy- benzonitrile | 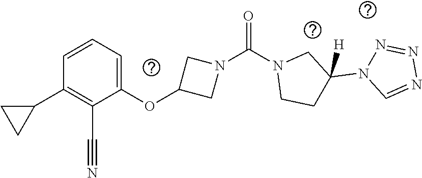 [SMILES: N#Cc1c(OC2CN(C(=O)N3CC[C@@H](n4cnnn4)C3)C2)cccc1C1CC1] | 380.4 [M + H]⁺ |
| 320 | Methyl 2,2-dimethyl-3-[3-[1- [(3R)-3-(tetrazol-1- yl)pyrrolidine-1- carbonyl]azetidin-3- yl]oxyphenyl]propanoate | 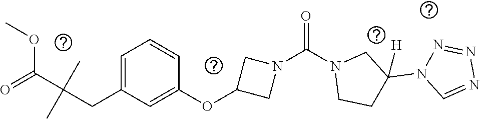 [SMILES: COC(=O)C(C)(C)Cc1cccc(OC2CN(C(=O)N3CCC(n4cnnn4)C3)C2)c1] | 429.2 [M + H]⁺ |
| 321 | [3-[6-(2-Chlorophenoxy)-3- pyridyl]azetidin-1-yl]-[(3S)-3- (tetrazol-1-yl)pyrrolidin-1- yl]methanone | 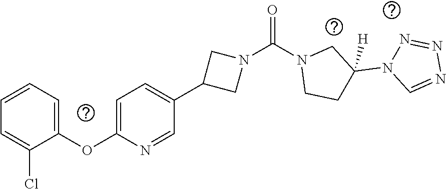 [SMILES: O=C(N1CC(c2ccc(Oc3ccccc3Cl)nc2)C1)N1CC[C@H](n2cnnn2)C1] | 426.2 [M + H]⁺ |
| 324 | 1-[4-[1-[(3R)-3-(Tetrazol-1- yl)pyrrolidine-1- carbonyl]azetidin-3- yl]phenyl]cyclopropane- carbonitrile | 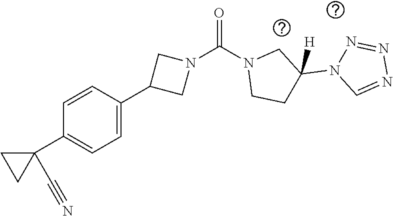 [SMILES: N#CC1(c2ccc(C3CN(C(=O)N4CC[C@@H](n5cnnn5)C4)C3)cc2)CC1] | 364.2 [M + H]⁺ |
| 325 | [3-[4-(4-Cyclopropylpyrimidin- 2-yl)oxyphenyl]azetidin-1-yl]- [(3R)-3-(tetrazol-1-yl)pyrrolidin- 1-yl]methanone | 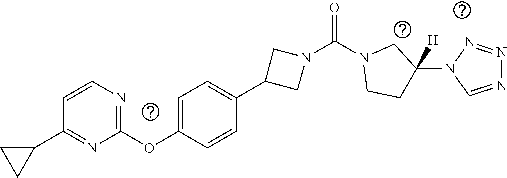 [SMILES: O=C(N1CC(c2ccc(Oc3nccc(C4CC4)n3)cc2)C1)N1CC[C@@H](n2cnnn2)C1] | 433.2 [M + H]⁺ |
| 326 | 1-[4-[1-[(3S)-3-(Tetrazol-1- yl)pyrrolidine-1- carbonyl]azetidin-3- yl]phenyl]cyclopropane- carbonitrile | 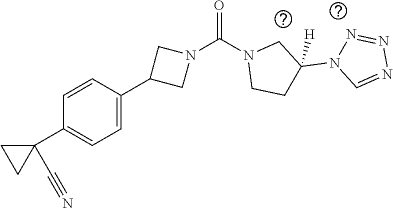 [SMILES: N#CC1(c2ccc(C3CN(C(=O)N4CC[C@H](n5cnnn5)C4)C3)cc2)CC1] | 364.2 [M + H]⁺ |
| 327 | Methyl 2,2-dimethyl-3-[3-[1- [(3S)-3-(tetrazol-1- yl)pyrrolidine-1- carbonyl]azetidin-3- yl]oxyphenyl]propanoate | 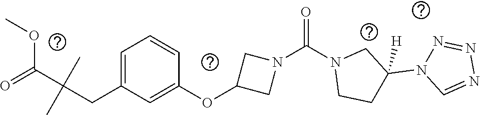 [SMILES: COC(=O)C(C)(C)Cc1cccc(OC2CN(C(=O)N3CC[C@H](n4cnnn4)C3)C2)c1] | 429.2 [M + H]⁺ |
| 328 | [3-[4-[3-(2,2- Dimethylpropyl)triazol-4- yl]phenyl]azetidin-1-yl]-[(3R)-3- (tetrazol-1-yl)pyrrolidin-1- yl]methanone | 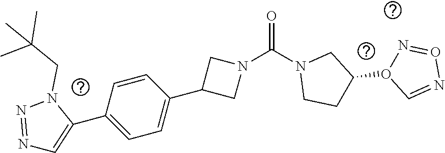 | 436.3 [M + H]⁺ |
| 336 | [6-[[2-Fluoro-4- (trifluoromethyl)phenyl]methyl- amino]-2-azaspiro[3.3]heptan-2- yl]-[3-(4H-1,2,4-triazol-3- yl)pyrrolidin-1-yl]methanone | 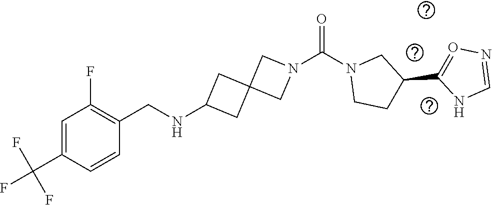 | 453.4 [M + H]⁺ |
| 337 | (3S)-1-[3-[4-[3- (Methylsulfonylmethyl)azetidin- 1-yl]phenyl]azetidine-1- carbonyl]pyrrolidine-3- carboxamide | 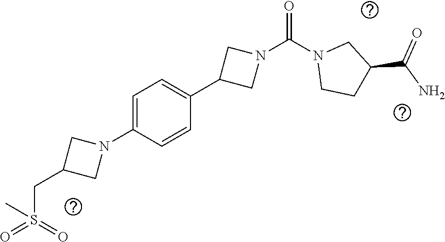 [SMILES: CS(=O)(=O)CC1CN(c2ccc(C3CN(C(=O)N4CC[C@H](C(N)=O)C4)C3)cc2)C1] | 421.2 [M + H]⁺ |
| 338 | rac-(3S)-1-[3-[5-[3- (Trifluoromethyl)pyrrolidin-1- yl]pyrazin-2-yl]azetidine-1- carbonyl]pyrrolidine-3- carboxamide | 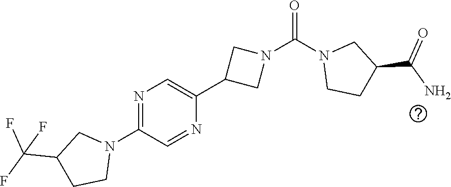 [SMILES: NC(=O)[C@H]1CCN(C(=O)N2CC(c3cnc(N4CCC(C(F)(F)F)C4)cn3)C2)C1] | 413.2 [M + H]⁺ |
| 339 | [(3S)-3-(1,2,4-Triazol-4- yl)pyrrolidin-1-yl]-[3-[4-[3- (trifluoromethyl)azetidin-1- yl]phenyl]azetidin-1- yl]methanone | 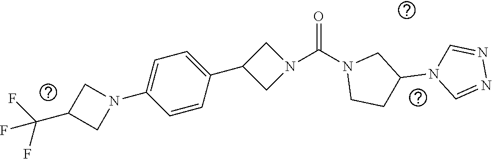 [SMILES: O=C(N1CC(c2ccc(N3CC(C(F)(F)F)C3)cc2)C1)N1CCC(n2cnnc2)C1] | 421.4 [M + H]⁺ |
| 340 | [6-[[2-Fluoro-4- (trifluoromethyl)phenyl]methyl- amino]-2-azaspiro[3.3]heptan-2- yl]-[(3S)-3-(1,2,4-triazol-4- yl)pyrrolidin-1-yl]methanone | 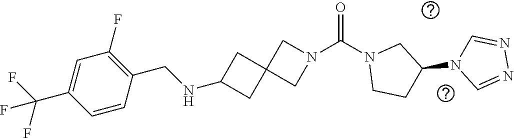 [SMILES: O=C(N1CC[C@H](n2cnnc2)C1)N1CC2(CC(NCc3ccc(C(F)(F)F)cc3F)C2)C1] | 453.5 [M + H]⁺ |
| 341 | [6-[(4,4-Difluoro-1- piperidyl)methyl]-2- azaspiro[3.3]heptan-2-yl]-[3- (4H-1,2,4-triazol-3- yl)pyrrolidin-1-yl]methanone | 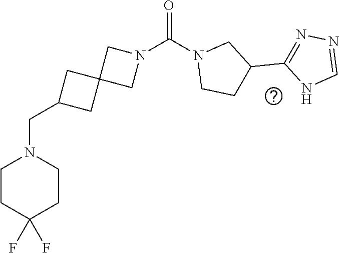 [SMILES: O=C(N1CCC(c2nnc[nH]2)C1)N1CC2(CC(CN3CCC(F)(F)CC3)C2)C1] | 395.3 [M + H]⁺ |
| 342 | [3-[(4,4- Difluorocyclohexyl) methoxy]azetidin-1-yl]-[3-(4H- 1,2,4-triazol-3-yl) pyrrolidin-1-yl]methanone | 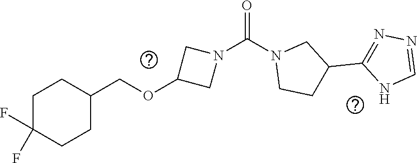 [SMILES: O=C(N1CC(OCC2CCC(F)(F)CC2)C1)N1CCC(c2nnc[nH]2)C1] | 370.4 [M + H]⁺ |
| 343 | N-[2-[(3S)-3-(1H-1,2,4-Triazol- 5-yl)pyrrolidine-1-carbonyl]-2- azaspiro[3.3]heptan-6-yl]-1- (trifluoromethyl)cyclopropane- carboxamide | 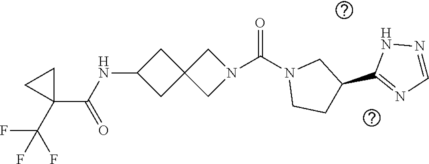 [SMILES: O=C(N1CC[C@H](c2ncn[nH]2)C1)N1CC2(CC(NC(=O)C3(C(F)(F)F)CC3)C2)C1] | 413.3 [M + H]⁺ |
| 345 | 1-[4-[1-[(3S)-3-(1H-1,2,4- Triazol-5-yl)pyrrolidine-1- carbonyl]azetidin-3- yl]phenyl]cyclopropane- carbonitrile | 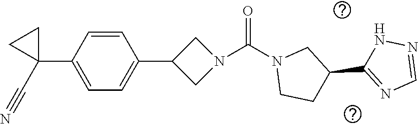 [SMILES: N#CC1(c2ccc(C3CN(C(=O)N4CC[C@H](c5ncn[nH]5)C4)C3)cc2)CC1] | 363.3 [M + H]⁺ |
| 346 | [(3S)-3-(1H-1,2,4-Triazol-5- yl)pyrrolidin-1-yl]-[3-[6-[1- (trifluoromethyl)cyclopropyl]-3- pyridyl]azetidin-1-yl]methanone | 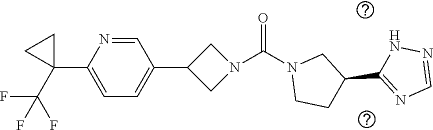 [SMILES: O=C(N1CC(c2ccc(C3(C(F)(F)F)CC3)nc2)C1)N1CC[C@H](c2ncn[nH]2)C1] | 407.3 [M + H]⁺ |
| 347 | [(3S)-3-(1H-1,2,4-Triazol-5- yl)pyrrolidin-1-yl]-[3-[2-[1- (trifluoromethyl)cyclopropyl] pyrimidin-5-yl]azetidin-1- yl]methanone | 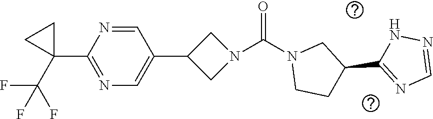 [SMILES: O=C(N1CC(c2cnc(C3(C(F)(F)F)CC3)nc2)C1)N1CC[C@H](c2ncn[nH]2)C1] | 408.3 [M + H]⁺ |
| 348 | [3-[(2-Fluoro-4-methylsulfonyl- phenyl)methoxy]azetidin-1-yl]- [(3S)-3-(1H-1,2,4-triazol-5- yl)pyrrolidin-1-yl]methanone | 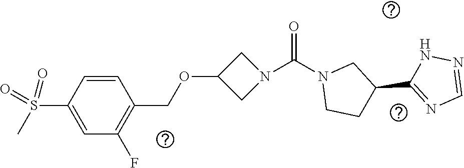 [SMILES: CS(=O)(=O)c1ccc(COC2CN(C(=O)N3CC[C@H](c4ncn[nH]4)C3)C2)c(F)c1] | 424.3 [M + H]⁺ |
| 349 | [(3S)-3-(1H-1,2,4-Triazol-5- yl)pyrrolidin-1-yl]-[6-[[6- (trifluoromethyl)-3-pyridyl]oxy]- 2-azaspiro[3.3]heptan-2- yl]methanone | 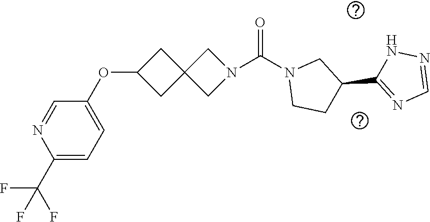 [SMILES: O=C(N1CC[C@H](c2ncn[nH]2)C1)N1CC2(CC(Oc3ccc(C(F)(F)F)nc3)C2)C1] | 423.3 [M + H]⁺ |
| 350 | [(3S)-3-(1H-1,2,4-Triazol-5- yl)pyrrolidin-1-yl]-[6-[[1- (trifluoromethyl)cyclopropyl] methoxy]-2-azaspiro[3.3]heptan- 2-yl]methanone | 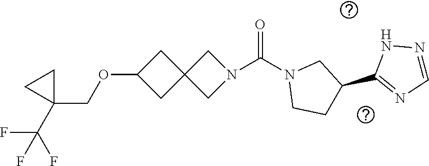 [SMILES: O=C(N1CC[C@H](c2ncn[nH]2)C1)N1CC2(CC(OCC3(C(F)(F)F)CC3)C2)C1] | 400.3 [M + H]⁺ |
| 351 | [(3S)-3-(1H-1,2,4-Triazol-5- yl)pyrrolidin-1-yl]-[2-[2- (trifluoromethyl)phenyl]sulfonyl- 2,6-diazaspiro[3.3]heptan-6- yl]methanone | 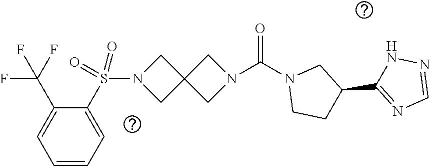 [SMILES: O=C(N1CC[C@H](c2ncn[nH]2)C1)N1CC2(C1)CN(S(=O)(=O)c1ccccc1C(F)(F)F)C2] | 471.2 [M + H]⁺ |
| 352 | [(3S)-3-(1H-1,2,4-Triazol-5- yl)pyrrolidin-1-yl]-[2-[3- (trifluoromethoxy) phenyl]sulfonyl- 2,6-diazaspiro[3.3]heptan-6- yl]methanone | 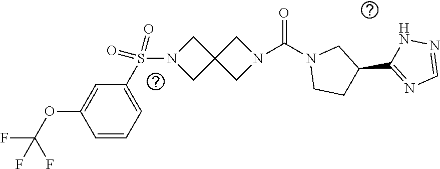 [SMILES: O=C(N1CC[C@H](c2ncn[nH]2)C1)N1CC2(C1)CN(S(=O)(=O)c1cccc(OC(F)(F)F)c1)C2] | 487.2 [M + H]⁺ |
| 353 | N-Methyl-2-[[2-[(3S)-3-(1H- 1,2,4-triazol-5-yl)pyrrolidine-1- carbonyl]-2,6- diazaspiro[3.3]heptan-6- yl]methyl]benzamide | 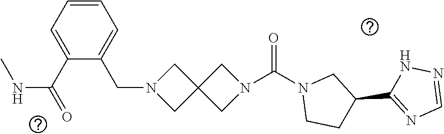 [SMILES: CNC(=O)c1ccccc1CN1CC2(C1)CN(C(=O)N1CC[C@H](c3ncn[nH]3)C1)C2] | 410.3 [M + H]⁺ |
| 354 | [(3S)-3-(1H-1,2,4-Triazol-5- yl)pyrrolidin-1-yl]-[2-[3- (trifluoromethyl)phenyl]sulfonyl- 2,6-diazaspiro[3.3]heptan-6- yl]methanone | 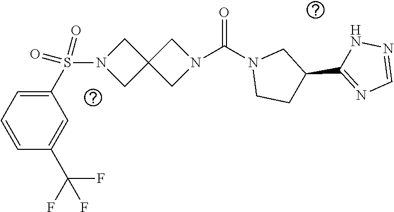 [SMILES: O=C(N1CC[C@H](c2ncn[nH]2)C1)N1CC2(C1)CN(S(=O)(=O)c1cccc(C(F)(F)F)c1)C2] | 471.2 [M + H]⁺ |
| 355 | [(3S)-3-(1,2,4-Triazol-4- yl)pyrrolidin-1-yl]-[3-[2-[1- (trifluoromethyl)cyclopropyl] pyrimidin-5-yl]azetidin-1- yl]methanone | 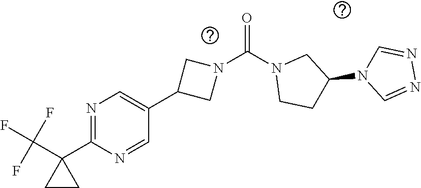 [SMILES: O=C(N1CC(c2cnc(C3(C(F)(F)F)CC3)nc2)C1)N1CC[C@H](n2cnnc2)C1] | 408.3 [M + H]⁺ |
| 356 | N-[[1-[(3S)-3-(1,2,4-Triazol-4- yl)pyrrolidine-1- carbonyl]azetidin-3-yl]methyl]- 3-(trifluoromethyl) benzenesulfonamide | 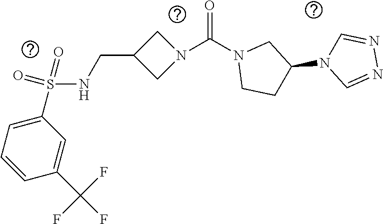 [SMILES: O=C(N1CC(CNS(=O)(=O)c2cccc(C(F)(F)F)c2)C1)N1CC[C@H](n2cnnc2)C1] | 459.3 [M + H]⁺ |
| 357 | [3-[4-(3- Dimethylphosphorylphenyl) phenyl]azetidin-1-yl]-[(3S)-3- (1,2,4-triazol-4-yl)pyrrolidin-1- yl]methanone | 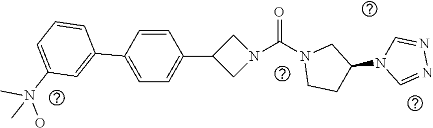 | 450.4 [M + H]⁺ |
| 358 | [(3S)-3-(1H-Triazol-5- yl)pyrrolidin-1-yl]-[3-[6- (trifluoromethyl)pyridazin-3- yl]oxyazetidin-1-yl]methanone | 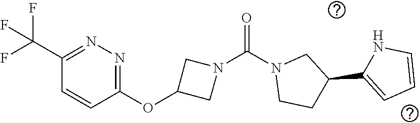 [SMILES: O=C(N1CC(Oc2ccc(C(F)(F)F)nn2)C1)N1CC[C@H](c2ccc[nH]2)C1] | 384.3 [M + H]⁺ |
| 360 | tert-Butyl N-[(3S)-1-[3-[4-(4- chloro-2-methylsulfonyl- phenyl)phenyl]azetidine-1- carbonyl]pyrrolidin-3- yl]carbamate | 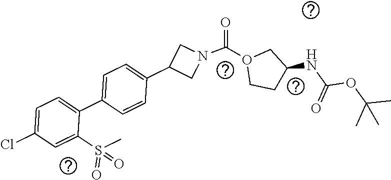 | 534.2 [M + H]⁺ |
| 362 | N-[[1-[(3S)-3-(1H-1,2,4-Triazol- 5-yl)pyrrolidine-1- carbonyl]azetidin-3-yl]methyl]- 3-(trifluoromethyl) benzenesulfonamide | 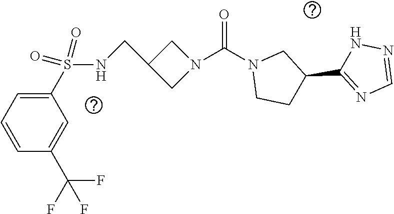 [SMILES: O=C(N1CC(CNS(=O)(=O)c2cccc(C(F)(F)F)c2)C1)N1CC[C@H](c2ncn[nH]2)C1] | 459.2 [M + H]⁺ |
| 363 | N-[[1-[(3S)-3-(1H-1,2,4-Triazol- 5-yl)pyrrolidine-1- carbonyl]azetidin-3-yl]methyl]- 4-(trifluoromethyl) benzenesulfonamide | 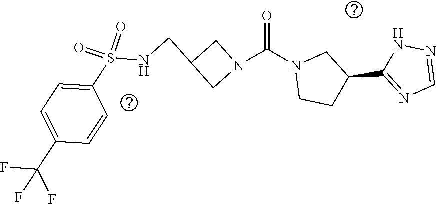 [SMILES: O=C(N1CC(CNS(=O)(=O)c2ccc(C(F)(F)F)cc2)C1)N1CC[C@H](c2ncn[nH]2)C1] | 459.2 [M + H]⁺ |
| 364 | [(3S)-3-(1H-1,2,4-Triazol-5- yl)pyrrolidin-1-yl]-[2-[4- (trifluoromethoxy) phenyl]sulfonyl- 2,6-diazaspiro[3.3]heptan-6- yl]methanone | 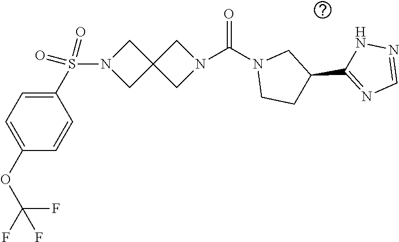 [SMILES: O=C(N1CC[C@H](c2ncn[nH]2)C1)N1CC2(C1)CN(S(=O)(=O)c1ccc(OC(F)(F)F)cc1)C2] | 487.2 [M + H]⁺ |
| 365 | [3-[5-(2,4-Difluorophenyl)-4H- 1,2,4-triazol-3-yl]azetidin-1-yl]- [(3S)-3-(1H-1,2,4-triazol-5- yl)pyrrolidin-1-yl]methanone | 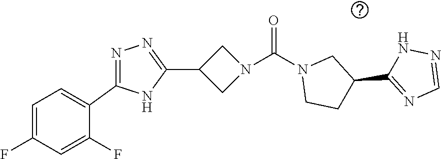 [SMILES: O=C(N1CC(c2nnc(-c3ccc(F)cc3F)[nH]2)C1)N1CC[C@H](c2ncn[nH]2)C1] | 401.2 [M + H]⁺ |
| 367 | (3S)-1-[3-[5-(2-Chloro-4- methylsulfonyl-phenyl)pyrazin- 2-yl]azetidine-1- carbonyl]pyrrolidine-3- carboxamide | 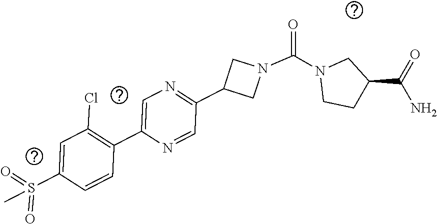 [SMILES: CS(=O)(=O)c1ccc(-c2cnc(C3CN(C(=O)N4CC[C@H](C(N)=O)C4)C3)cn2)c(Cl)c1] | 464.1 [M + H]⁺ |
| 368 | (3R)-1-[3-[[2-Fluoro-4- (trifluoromethyl)phenyl] methoxy]azetidine-1- carbonyl]pyrrolidine-3- carboxamide | 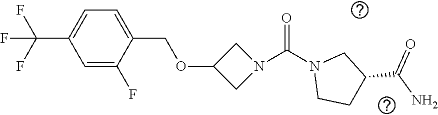 [SMILES: NC(=O)[C@@H]1CCN(C(=O)N2CC(OCc3ccc(C(F)(F)F)cc3F)C2)C1] | 390.2 [M + H]⁺ |
| 369 | (3R)-1-[6-[[6-(Trifluoromethyl)- 3-pyridyl]oxy]-2- azaspiro[3.3]heptane-2- carbonyl]pyrrolidine-3- carboxamide | 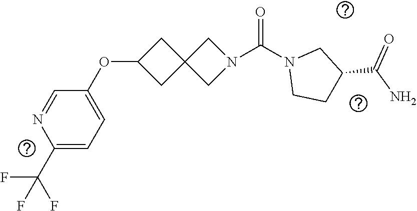 [SMILES: NC(=O)[C@@H]1CCN(C(=O)N2CC3(CC(Oc4ccc(C(F)(F)F)nc4)C3)C2)C1] | 399.2 [M + H]⁺ |
| 370 | (3S)-1-[3-[[2-Fluoro-4- (trifluoromethyl)phenyl]methyl- amino]azetidine-1- carbonyl]pyrrolidine-3- carboxamide | 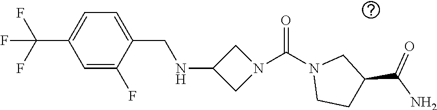 [SMILES: NC(=O)[C@H]1CCN(C(=O)N2CC(NCc3ccc(C(F)(F)F)cc3F)C2)C1] | 389.2 [M + H]⁺ |
| 372 | [3-[[2-Fluoro-4- (trifluoromethyl)phenyl]methyl- amino]azetidin-1-yl]-[(3R)-3- (1H-1,2,4-triazol-5- yl)pyrrolidin-1-yl]methanone | 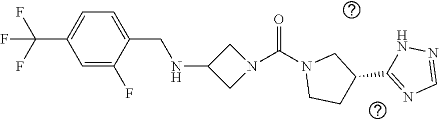 [SMILES: O=C(N1CC(NCc2ccc(C(F)(F)F)cc2F)C1)N1CC[C@@H](c2ncn[nH]2)C1] | 413.2 [M + H]⁺ |
| 373 | 2-[(3S)-3-(1H-1,2,4-Triazol-5- yl)pyrrolidine-1-carbonyl]-N- [[1-(trifluoromethyl) cyclopropyl]methyl]- 2-azaspiro[3.3]heptane-6- carboxamide | 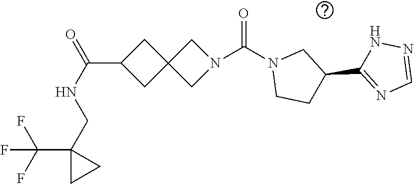 [SMILES: O=C(NCC1(C(F)(F)F)CC1)C1CC2(C1)CN(C(=O)N1CC[C@H](c3ncn[nH]3)C1)C2] | 427.3 [M + H]⁺ |
| 374 | [(3S)-3-(1,2,4-Triazol-4- yl)pyrrolidin-1-yl]-[6-[[[1- (trifluoromethyl)cyclopropyl] amino]methyl]-2- azaspiro[3.3]heptan-2- yl]methanone | 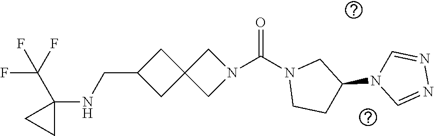 [SMILES: O=C(N1CC[C@H](n2cnnc2)C1)N1CC2(CC(CNC3(C(F)(F)F)CC3)C2)C1] | 399.4 [M + H]⁺ |
| 375 | [3-[[2-Fluoro-4- (trifluoromethyl)phenyl]methyl- amino]azetidin-1-yl]-[(3R)-3- (tetrazol-1-yl)pyrrolidin-1- yl]methanone | 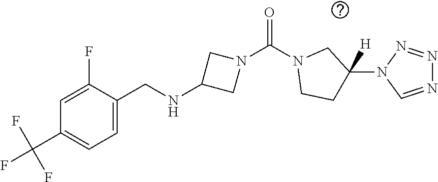 [SMILES: O=C(N1CC(NCc2ccc(C(F)(F)F)cc2F)C1)N1CC[C@@H](n2cnnn2)C1] | 414.2 [M + H]⁺ |
| 376 | [3-[4-(Benzene- sulfonyl)phenyl]azetidin- 1-yl]-[(3S)-3-(tetrazol-1- yl)pyrrolidin-1-yl]methanone | 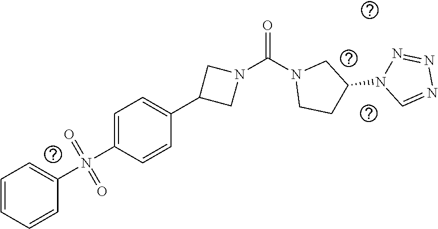 | 439.2 [M + H]⁺ |
| 378 | [3-[6-(2- Chlorophenoxy)pyridazin-3- yl]azetidin-1-yl]-[(3S)-3- (tetrazol-1-yl)pyrrolidin-1- yl]methanone | 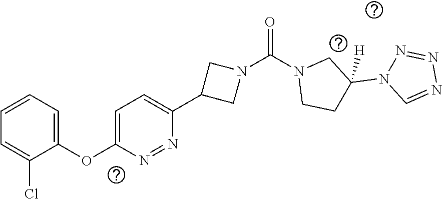 [SMILES: O=C(N1CC(c2ccc(Oc3ccccc3Cl)nn2)C1)N1CC[C@H](n2cnnn2)C1] | 427.2 [M + H]⁺ |
| 379 | [3-[[2-Fluoro-4- (trifluoromethyl)phenyl]methyl- amino]azetidin-1-yl]-[(3S)-3- (tetrazol-1-yl)pyrrolidin-1- yl]methanone | 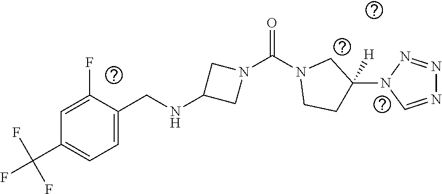 [SMILES: O=C(N1CC(NCc2ccc(C(F)(F)F)cc2F)C1)N1CC[C@H](n2cnnn2)C1] | 414.2 [M + H]⁺ |
| 380 | Methyl 2-methyl-2-[3-[1-[(3S)- 3-(tetrazol-1-yl)pyrrolidine-1- carbonyl]azetidin-3- yl]oxyphenyl]propanoate | 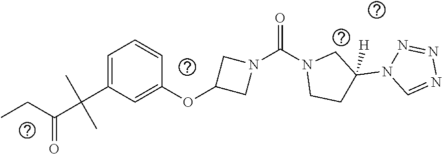 [SMILES: CCC(=O)C(C)(C)c1cccc(OC2CN(C(=O)N3CC[C@H](n4cnnn4)C3)C2)c1] | 415.2 [M + H]⁺ |
| 381 | [2-[4-Fluoro-2- (trifluoromethyl)phenyl]sulfonyl- 2,6-diazaspiro[3.3]heptan-6-yl]- [(3S)-3-(tetrazol-1-yl)pyrrolidin- 1-yl]methanone | 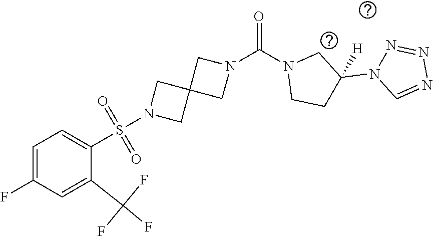 [SMILES: O=C(N1CC[C@H](n2cnnn2)C1)N1CC2(C1)CN(S(=O)(=O)c1ccc(F)cc1C(F)(F)F)C2] | 490.1 [M + H]⁺ |
| 382 | Methyl 2-methyl-2-[3-[1-[(3R)- 3-(tetrazol-1-yl)pyrrolidine-1- carbonyl]azetidin-3- yl]oxyphenyl]propanoate | 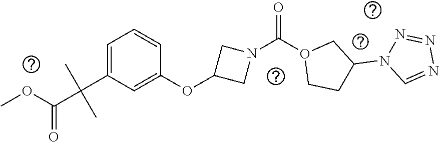 | 415.2 [M + H]⁺ |
| 383 | [(3R)-3-(Tetrazol-1- yl)pyrrolidin-1-yl]-[3-[4-[4- (trifluoromethyl)pyrimidin-2- yl]oxyphenyl]azetidin-1- yl]methanone | 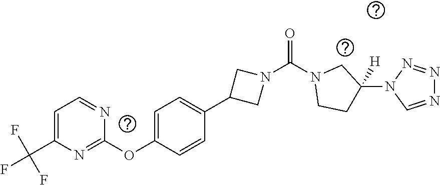 [SMILES: O=C(N1CC(c2ccc(Oc3nccc(C(F)(F)F)n3)cc2)C1)N1CC[C@H](n2cnnn2)C1] | 461.2 [M + H]⁺ |
| 384 | [2-[4-Fluoro-3- (trifluoromethyl)phenyl]sulfonyl- 2,6-diazaspiro[3.3]heptan-6-yl]- [(3S)-3-(tetrazol-1-yl)pyrrolidin- 1-yl]methanone | 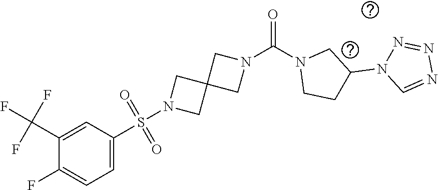 [SMILES: O=C(N1CCC(n2cnnn2)C1)N1CC2(C1)CN(S(=O)(=O)c1ccc(F)c(C(F)(F)F)c1)C2] | 490.1 [M + H]⁺ |
| 385 | [3-[(2-Fluoro-4-methylsulfonyl- phenyl)methoxy]azetidin-1-yl]- [(3S)-3-(tetrazol-1-yl)pyrrolidin- 1-yl]methanone | 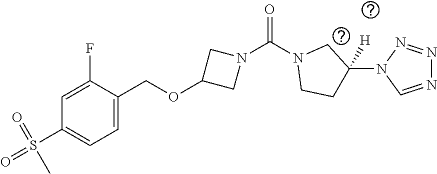 [SMILES: CS(=O)(=O)c1ccc(COC2CN(C(=O)N3CC[C@H](n4cnnn4)C3)C2)c(F)c1] | 425.2 [M + H]⁺ |
| 386 | [2-[4-Fluoro-2- (trifluoromethyl)phenyl]sulfonyl- 2,6-diazaspiro[3.3]heptan-6-yl]- [(3R)-3-(tetrazol-1-yl)pyrrolidin- 1-yl]methanone | 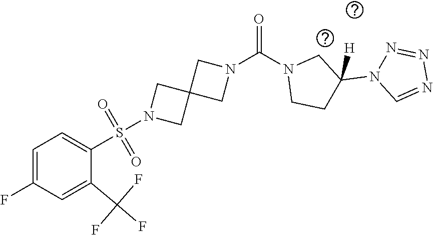 [SMILES: O=C(N1CC[C@@H](n2cnnn2)C1)N1CC2(C1)CN(S(=O)(=O)c1ccc(F)cc1C(F)(F)F)C2] | 490.2 [M + H]⁺ |
| 388 | [3-[4- (Dimethylphosphorylmethyl) phenyl]azetidin-1-yl]-[(3S)-3- (1H-triazol-5-yl)pyrrolidin-1- yl]methanone | 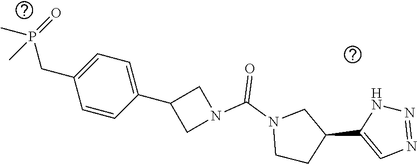 [SMILES: CP(C)(=O)Cc1ccc(C2CN(C(=O)N3CC[C@H](c4cnn[nH]4)C3)C2)cc1] | 388.3 [M + H]⁺ |
| 391 | [3-[4-(2-Chloro-4- methylsulfonyl- phenyl)phenyl]azetidin-1-yl]- [(3S)-3-hydroxypyrrolidin-1- yl]methanone | 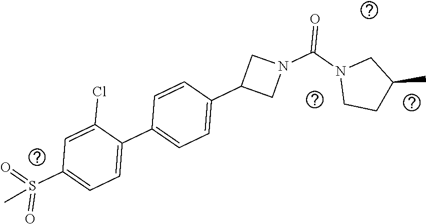 | 435.1 [M + H]⁺ |
| 395 | (3-Pyrrolidin-1- ylsulfonylpyrrolidin-1-yl)- [3-[4-[1-(trifluoromethyl) cyclopropyl]phenyl] azetidin-1-yl]methanone | 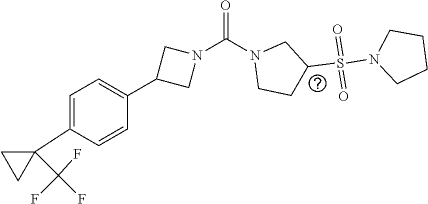 [SMILES: O=C(N1CC(c2ccc(C3(C(F)(F)F)CC3)cc2)C1)N1CCC(S(=O)(=O)N2CCCC2)C1] | 472.2 [M + H]⁺ |
| 396 | Morpholino-[3-[4-[1- (trifluoromethyl)cyclopropyl] phenyl]azetidin-1-yl]methanone | 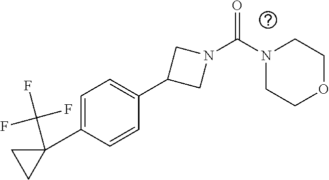 [SMILES: O=C(N1CCOCC1)N1CC(c2ccc(C3(C(F)(F)F)CC3)cc2)C1] | 355.1 [M + H]⁺ |
| 397 | [3-[5-(4-Chloro-2-fluoro- phenoxy)pyrazin-2-yl]azetidin- 1-yl]-morpholino-methanone | 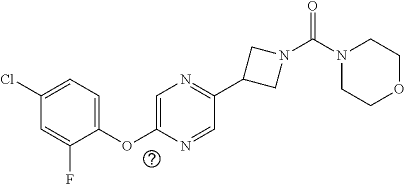 [SMILES: O=C(N1CCOCC1)N1CC(c2cnc(Oc3ccc(Cl)cc3F)cn2)C1] | 393.1 [M + H]⁺ |
| 398 | [3-[[rac-(1S,5R)-6,6-difluoro-3- bicyclo[3.1.0]hexanyl]methoxy] azetidin-1-yl]-[3-(4H-1,2,4- triazol-3-yl)pyrrolidin-1- yl]methanone | 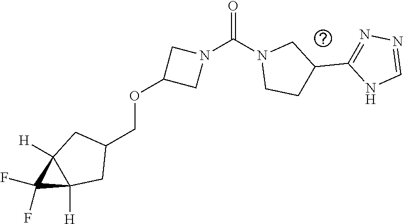 [SMILES: O=C(N1CC(OCC2C[C@@H]3[C@H](C2)C3(F)F)C1)N1CCC(c2nnc[nH]2)C1] | 368.3 [M + H]⁺ |
| 399 | [6-[[2-Fluoro-4- (trifluoromethyl)phenyl]methyl- amino]-2-azaspiro[3.3]heptan-2- yl]-[(3S)-3-(4H-1,2,4-triazol-3- yl)pyrrolidin-1-yl]methanone | 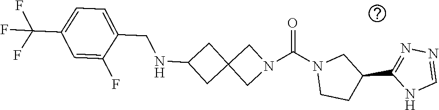 [SMILES: O=C(N1CC[C@H](c2nnc[nH]2)C1)N1CC2(CC(NCc3ccc(C(F)(F)F)cc3F)C2)C1] | 453.3 [M + H]⁺ |
| 401 | [7-[[2-Fluoro-4- (trifluoromethyl)phenyl]methyl]- 2,7-diazaspiro[3.5]nonan-2-yl]- [(3R)-3-(4H-1,2,4-triazol-3- yl)pyrrolidin-1-yl]methanone | 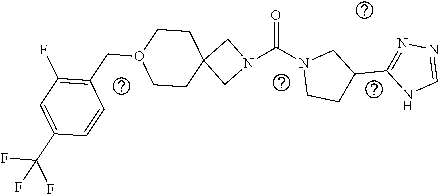 | 467.3 [M + H]⁺ |
| 403 | [7-[2-Fluoro-4- (trifluoromethyl)phenoxy]-2- azaspiro[3.5]nonan-2-yl]-[(3R)- 3-(4H-1,2,4-triazol-3- yl)pyrrolidin-1-yl]methanone | 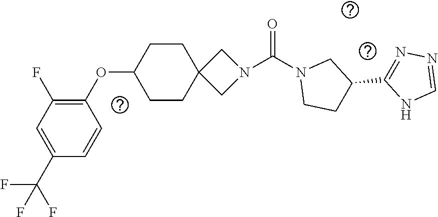 [SMILES: O=C(N1CC[C@@H](c2nnc[nH]2)C1)N1CC2(CCC(Oc3ccc(C(F)(F)F)cc3F)CC2)C1] | 468.2 [M + H]⁺ |
| 404 | (3S)-1-[3-[5-(4-Chloro-2-fluoro- phenyl)pyrimidin-2-yl]azetidine- 1-carbonyl]pyrrolidine-3- carboxamide | 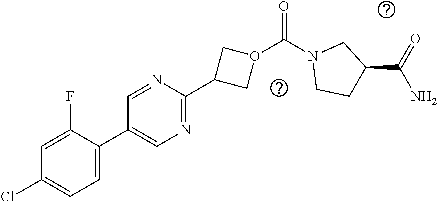 | 404.1 [M + H]⁺ |
| 408 | (3S)-1-[3-[6-(2-Chloro-4- methylsulfonyl-phenyl)-3- pyridyl]azetidine-1- carbonyl]pyrrolidine-3- carboxamide | 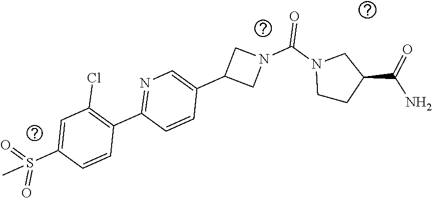 [SMILES: CS(=O)(=O)c1ccc(-c2ccc(C3CN(C(=O)N4CC[C@H](C(N)=O)C4)C3)cn2)c(Cl)c1] | 463.1 [M + H]⁺ |
| 409 | (3S)-1-[3-[6-(4-Chloro-2- methylsulfonyl-phenyl)-3- pyridyl]azetidine-1- carbonyl]pyrrolidine-3- carboxamide | 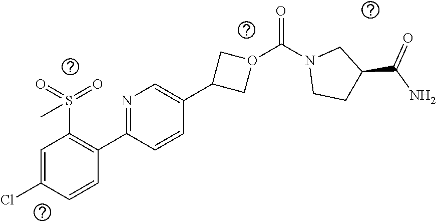 | 463.1 [M + H]⁺ |
| 410 | [3-[4-(6-Chloro-4- methylsulfonyl-3- pyridyl)phenyl]azetidin-1-yl]- [(3S)-3-hydroxypyrrolidin-1- yl]methanone | 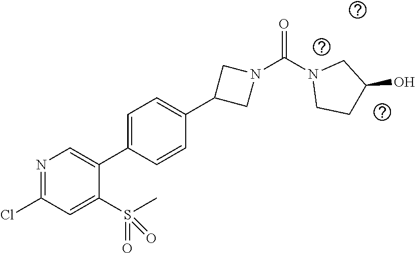 [SMILES: CS(=O)(=O)c1cc(Cl)ncc1-c1ccc(C2CN(C(=O)N3CC[C@H](O)C3)C2)cc1] | 436.1 [M + H]⁺ |
| 412 | [(3S)-3-Aminopyrrolidin-1-yl]- [3-[4-(4-chloro-2- methylsulfonyl- phenyl)phenyl]azetidin-1- yl]methanone | 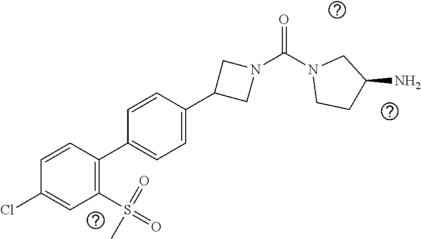 [SMILES: CS(=O)(=O)c1cc(Cl)ccc1-c1ccc(C2CN(C(=O)N3CC[C@H](N)C3)C2)cc1] | 434.1 [M + H]⁺ |
| 414 | [2-[[2-Fluoro-4- (trifluoromethyl)phenyl]methyl]- 2,7-diazaspiro[3.5]nonan-7-yl]- [(3R)-3-(4H-1,2,4-triazol-3- yl)pyrrolidin-1-yl]methanone | 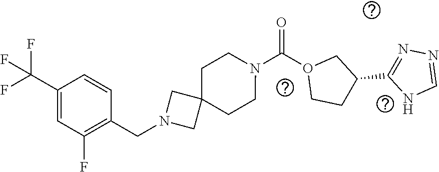 | 467.3 [M + H]⁺ |
| 416 | (3R)-1-[7-[[2-Fluoro-4- (trifluoromethyl)phenyl]methyl]- 2,7-diazaspiro[3.5]nonane-2- carbonyl]pyrrolidine-3- carboxamide | 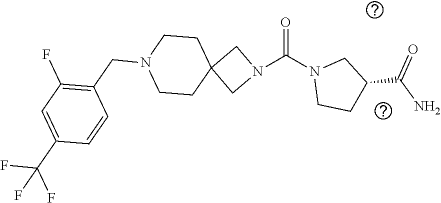 [SMILES: NC(=O)[C@@H]1CCN(C(=O)N2CC3(CCN(Cc4ccc(C(F)(F)F)cc4F)CC3)C2)C1] | 443.3 [M + H]⁺ |
| 418 | (3R)-1-[7-[2-Fluoro-4- (trifluoromethyl)phenoxy]-2- azaspiro[3.5]nonane-2- carbonyl]pyrrolidine-3- carboxamide | 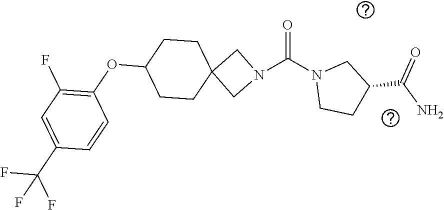 [SMILES: NC(=O)[C@@H]1CCN(C(=O)N2CC3(CCC(Oc4ccc(C(F)(F)F)cc4F)CC3)C2)C1] | 444.2 [M + H]⁺ |
| 419 | (3S)-1-[2-[[2-Fluoro-4- (trifluoromethyl)phenyl]methyl]- 2,7-diazaspiro[3.5]nonane-7- carbonyl]pyrrolidine-3- carboxamide | 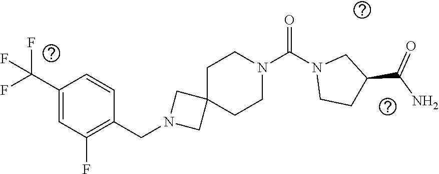 [SMILES: NC(=O)[C@H]1CCN(C(=O)N2CCC3(CC2)CN(Cc2ccc(C(F)(F)F)cc2F)C3)C1] | 443.3 [M + H]⁺ |
| 420 | (3R)-1-[2-[[2-Fluoro-4- (trifluoromethyl)phenyl]methyl]- 2,7-diazaspiro[3.5]nonane-7- carbonyl]pyrrolidine-3- carboxamide | 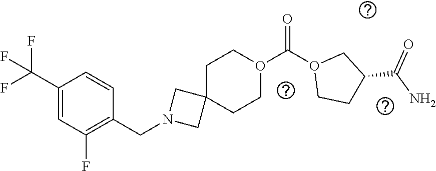 | 443.2 [M + H]⁺ |
| 421 | [7-[2-Fluoro-4- (trifluoromethyl)phenyl]sulfonyl- 2,7-diazaspiro[3.5]nonan-2-yl]- [(3S)-3-(1H-1,2,4-triazol-5- yl)pyrrolidin-1-yl]methanone | 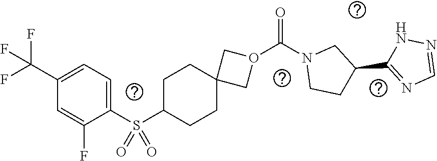 | 517.2 [M + H]⁺ |
| 422 | (3S)-1-[7-[2-Fluoro-4- (trifluoromethyl)phenyl]sulfonyl- 2,7-diazaspiro[3.5]nonane-2- carbonyl]pyrrolidine-3- carboxamide | 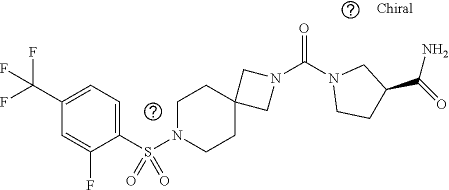 [SMILES: NC(=O)[C@H]1CCN(C(=O)N2CC3(CCN(S(=O)(=O)c4ccc(C(F)(F)F)cc4F)CC3)C2)C1] | 493.2 [M + H]⁺ |
| 423 | [6-[(3,5-Difluoro-2- pyridyl)methyl]-2- azaspiro[3.3]heptan-2-yl]-[(3S)- 3-(tetrazol-1-yl)pyrrolidin-1- yl]methanone | 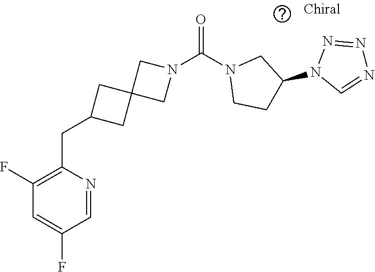 [SMILES: O=C(N1CC[C@H](n2cnnn2)C1)N1CC2(CC(Cc3ncc(F)cc3F)C2)C1] | 390.2 [M + H]⁺ |
| 425 | [6-[(5-Fluoro-3-pyridyl)methyl]- 2-azaspiro[3.3]heptan-2-yl]- [(3S)-3-(tetrazol-1-yl)pyrrolidin- 1-yl]methanone | 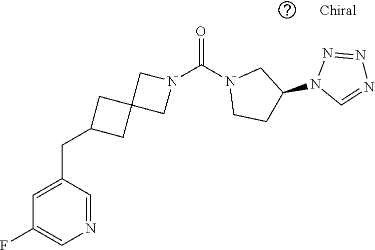 [SMILES: O=C(N1CC[C@H](n2cnnn2)C1)N1CC2(CC(Cc3cncc(F)c3)C2)C1] | 372.2 [M + H]⁺ |
| 426 | [6-[(3,5-Difluoro-2- pyridyl)methyl]-2- azaspiro[3.3]heptan-2-yl]-[(3R)- 3-(tetrazol-1-yl)pyrrolidin-1- yl]methanone | 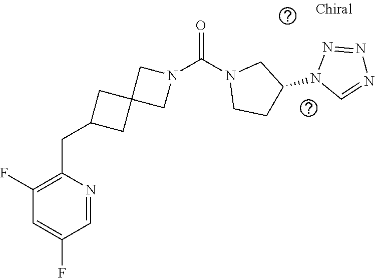 [SMILES: O=C(N1CC[C@@H](n2cnnn2)C1)N1CC2(CC(Cc3ncc(F)cc3F)C2)C1] | 390.1 [M + H]⁺ |
| 428 | [6-[(5-Fluoro-3-pyridyl)methyl]- 2-azaspiro[3.3]heptan-2-yl]- [(3R)-3-(tetrazol-1-yl)pyrrolidin- 1-yl]methanone | 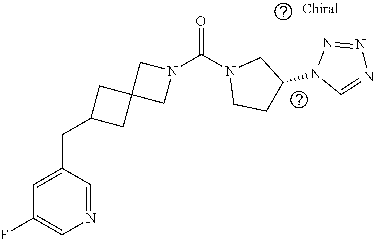 [SMILES: O=C(N1CC[C@@H](n2cnnn2)C1)N1CC2(CC(Cc3cncc(F)c3)C2)C1] | 372.1 [M + H]⁺ |
| 429 | [3-(4H-1,2,4-Triazol-3- yl)pyrrolidin-1-yl]-[7-[2- (trifluoromethyl)pyrimidin-4- yl]oxy-2-azaspiro[3.5]nonan-2- yl]methanone | 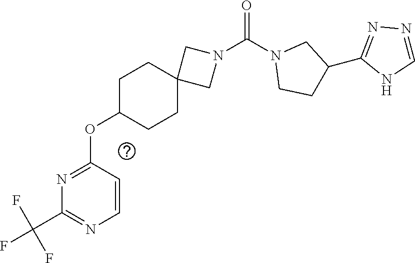 [SMILES: O=C(N1CCC(c2nnc[nH]2)C1)N1CC2(CCC(Oc3ccnc(C(F)(F)F)n3)CC2)C1] | 452.3 [M + H]⁺ |
| 430 | N-[2-[3-(4H-1,2,4-Triazol-3- yl)pyrrolidine-1-carbonyl]-2- azaspiro[3.5]nonan-7-yl]-3- (trifluoromethoxy)benzene- sulfonamide | 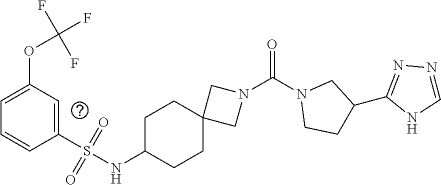 [SMILES: O=C(N1CCC(c2nnc[nH]2)C1)N1CC2(CCC(NS(=O)(=O)c3cccc(OC(F)(F)F)c3)CC2)C1] | 529.2 [M + H]⁺ |
| 431 | (3S)-1-[3-[4-(4-Chloro-2-fluoro- phenyl)-3-methylsulfonyl- phenyl]azetidine-1- carbonyl]pyrrolidine-3- carboxamide | 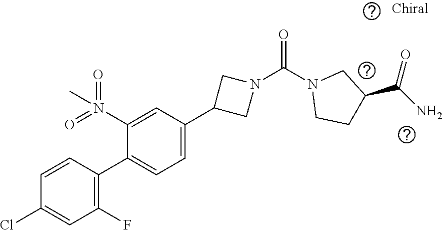 | 480.1 [M + H]⁺ |
| 432 | N-[2-[(3S)-3-(1H-1,2,4-Triazol- 5-yl)pyrrolidine-1-carbonyl]-2- azaspiro[3.3]heptan-6-yl]-2- (trifluoromethyl)benzene- sulfonamide | 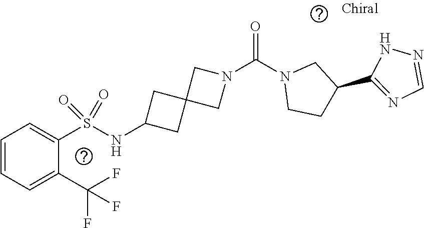 [SMILES: O=C(N1CC[C@H](c2ncn[nH]2)C1)N1CC2(CC(NS(=O)(=O)c3ccccc3C(F)(F)F)C2)C1] | 485.2 [M + H]⁺ |
| 433 | N-[2-[(3S)-3-(1H-1,2,4-Triazol- 5-yl)pyrrolidine-1-carbonyl]-2- azaspiro[3.3]heptan-6-yl]-3- (trifluoromethyl)benzene- sulfonamide | 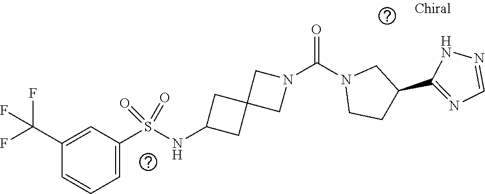 [SMILES: O=C(N1CC[C@H](c2ncn[nH]2)C1)N1CC2(CC(NS(=O)(=O)c3cccc(C(F)(F)F)c3)C2)C1] | 485.2 [M + H]⁺ |
| 434 | N-[2-[(3S)-3-(1H-1,2,4-Triazol- 5-yl)pyrrolidine-1-carbonyl]-2- azaspiro[3.3]heptan-6-yl]-4- (trifluoromethyl)benzene- sulfonamide | 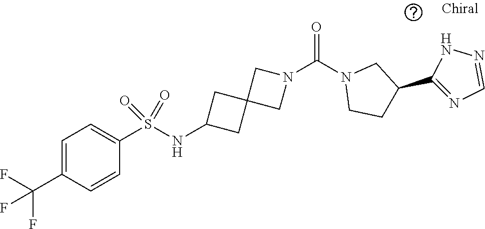 [SMILES: O=C(N1CC[C@H](c2ncn[nH]2)C1)N1CC2(CC(NS(=O)(=O)c3ccc(C(F)(F)F)cc3)C2)C1] | 485.2 [M + H]⁺ |
| 436 | [(3S)-3-(1H-1,2,4-Triazol-5- yl)pyrrolidin-1-yl]-[6-[[6- (trifluoromethyl)-3- pyridyl]methyl]-2- azaspiro[3.3]heptan-2- yl]methanone | 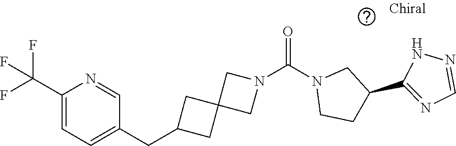 [SMILES: O=C(N1CC[C@H](c2ncn[nH]2)C1)N1CC2(CC(Cc3ccc(C(F)(F)F)nc3)C2)C1] | 421.2 [M + H]⁺ |
| 437 | [(3S)-3-(1H-1,2,4-Triazol-5- yl)pyrrolidin-1-yl]-[6-[[5- (trifluoromethyl)pyrazin-2- yl]methyl]-2- azaspiro[3.3]heptan-2- yl]methanone | 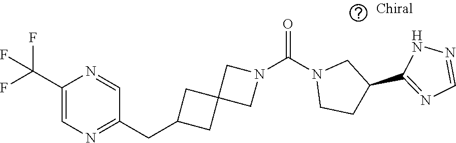 [SMILES: O=C(N1CC[C@H](c2ncn[nH]2)C1)N1CC2(CC(Cc3cnc(C(F)(F)F)cn3)C2)C1] | 422.2 [M + H]⁺ |
| 438 | (3S)-1-[6-[[2-(Trifluoro- methyl)phenyl]sulfonyl- amino]-2-azaspiro[3.3]heptane- 2-carbonyl]pyrrolidine-3- carboxamide | 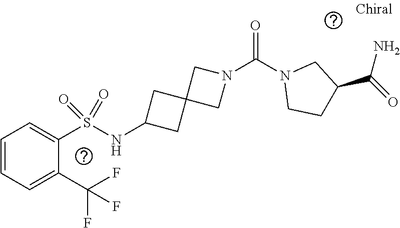 [SMILES: NC(=O)[C@H]1CCN(C(=O)N2CC3(CC(NS(=O)(=O)c4ccccc4C(F)(F)F)C3)C2)C1] | 461.2 [M + H]⁺ |
| 439 | (3S)-1-[6-[[3-(Trifluoro- methyl)phenyl]sulfonyl- amino]-2-azaspiro[3.3]heptane- 2-carbonyl]pyrrolidine-3- carboxamide | 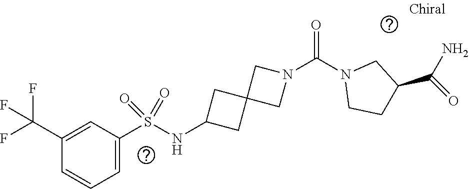 [SMILES: NC(=O)[C@H]1CCN(C(=O)N2CC3(CC(NS(=O)(=O)c4cccc(C(F)(F)F)c4)C3)C2)C1] | 461.2 [M + H]⁺ |
| 440 | (3S)-1-[6-[[4-(Trifluoro- methyl)phenyl]sulfonyl- amino]-2-azaspiro[3.3]heptane- 2-carbonyl]pyrrolidine-3- carboxamide | 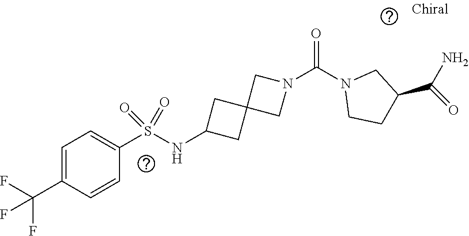 [SMILES: NC(=O)[C@H]1CCN(C(=O)N2CC3(CC(NS(=O)(=O)c4ccc(C(F)(F)F)cc4)C3)C2)C1] | 461.2 [M + H]⁺ |
| 441 | (3S)-1-[6-(2,2- Dimethylpropylsulfonylamino)- 2-azaspiro[3.3]heptane-2- carbonyl]pyrrolidine-3- carboxamide | 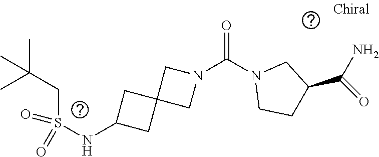 [SMILES: CC(C)(C)CS(=O)(=O)NC1CC2(C1)CN(C(=O)N1CC[C@H](C(N)=O)C1)C2] | 387.2 [M + H]⁺ |
| 442 | (3S)-1-[3-[5-(2-Chloro-4- methylsulfonyl- phenyl)pyrimidin-2-yl]azetidine- 1-carbonyl]pyrrolidine-3- carboxamide | 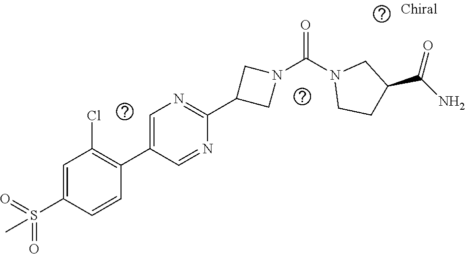 [SMILES: CS(=O)(=O)c1ccc(-c2cnc(C3CN(C(=O)N4CC[C@H](C(N)=O)C4)C3)nc2)c(Cl)c1] | 464.1 [M + H]⁺ |
| 443 | (3S)-1-[3-[2-(2-Chloro-4- methylsulfonyl- phenyl)pyrimidin-5-yl]azetidine- 1-carbonyl]pyrrolidine-3- carboxamide | 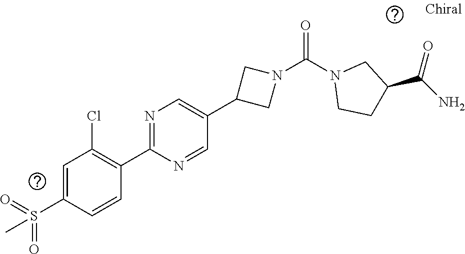 [SMILES: CS(=O)(=O)c1ccc(-c2ncc(C3CN(C(=O)N4CC[C@H](C(N)=O)C4)C3)cn2)c(Cl)c1] | 464.1 [M + H]⁺ |
| 444 | [3-[6-(1,1-Dioxo-1,4-thiazinan- 4-yl)-3-pyridyl]azetidin-1-yl]-[3- (1H-1,2,4-triazol-5- yl)pyrrolidin-1-yl]methanone | 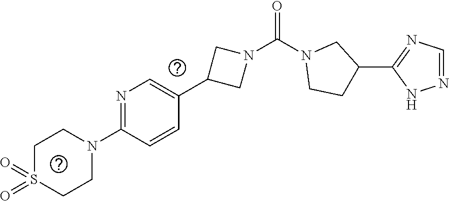 [SMILES: O=C(N1CC(c2ccc(N3CCS(=O)(=O)CC3)nc2)C1)N1CCC(c2ncn[nH]2)C1] | 432.3 [M + H]⁺ |
| 446 | [3-(4H-1,2,4-Triazol-3- yl)pyrrolidin-1-yl]-[6-[[6- (trifluoromethyl)-3- pyridyl]methyl]-2- azaspiro[3.3]heptan-2- yl]methanone | 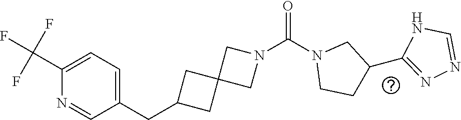 [SMILES: O=C(N1CCC(c2nnc[nH]2)C1)N1CC2(CC(Cc3ccc(C(F)(F)F)nc3)C2)C1] | 421.2 [M + H]⁺ |
| 447 | 1-[6-[[6-(Trifluoromethyl)-3- pyridyl]methyl]-2- azaspiro[3.3]heptane-2- carbonyl]pyrrolidine-3- sulfonamide | 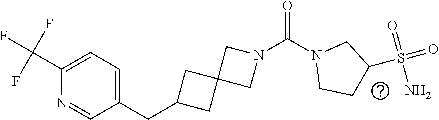 [SMILES: NS(=O)(=O)C1CCN(C(=O)N2CC3(CC(Cc4ccc(C(F)(F)F)nc4)C3)C2)C1] | 433.1 [M + H]⁺ |
| 448 | 1-[6-[[6-(Trifluoromethyl)-3- pyridyl]methyl]-2- azaspiro[3.3]heptane-2- carbonyl]piperidine-3- carboxamide | 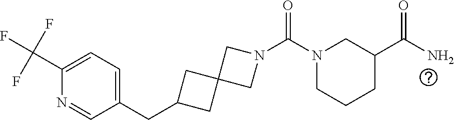 [SMILES: NC(=O)C1CCCN(C(=O)N2CC3(CC(Cc4ccc(C(F)(F)F)nc4)C3)C2)C1] | 411.2 [M + H]⁺ |
| 449 | [3-[5-(4- Chlorophenoxy)pyrazin-2- yl]azetidin-1-yl]-[(3S)-3- (tetrazol-1-yl)pyrrolidin-1- yl]methanone | 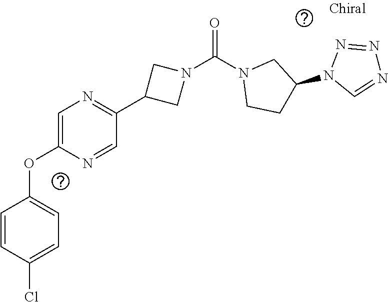 [SMILES: O=C(N1CC(c2cnc(Oc3ccc(Cl)cc3)cn2)C1)N1CC[C@H](n2cnnn2)C1] | 427.1 [M + H]⁺ |
| 450 | [(3S)-3-(Tetrazol-1- yl)pyrrolidin-1-yl]-[6-[4- (trifluoromethyl)phenyl]-2- azaspiro[3.3]heptan-2- yl]methanone | 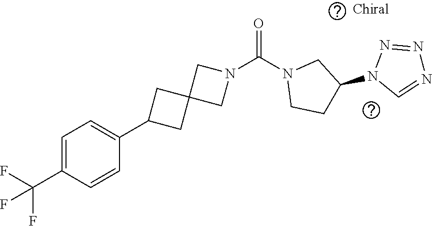 [SMILES: O=C(N1CC[C@H](n2cnnn2)C1)N1CC2(CC(c3ccc(C(F)(F)F)cc3)C2)C1] | 407.2 [M + H]⁺ |
| 451 | [(3S)-3-(Tetrazol-1- yl)pyrrolidin-1-yl]-[6-[2- (trifluoromethyl)pyrimidin-5-yl]- 2-azaspiro[3.3]heptan-2- yl]methanone | 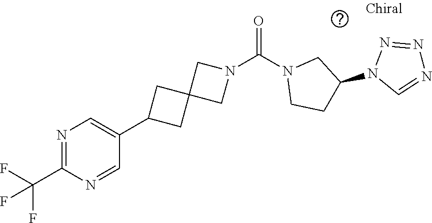 [SMILES: O=C(N1CC[C@H](n2cnnn2)C1)N1CC2(CC(c3cnc(C(F)(F)F)nc3)C2)C1] | 409.1 [M + H]⁺ |
| 452 | [3-(4H-1,2,4-Triazol-3- yl)pyrrolidin-1-yl]-[7-[6- (trifluoromethyl)pyrazin-2- yl]oxy-2-azaspiro[3.5]nonan-2- yl]methanone | 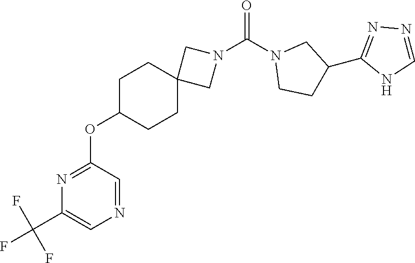 [SMILES: O=C(N1CCC(c2nnc[nH]2)C1)N1CC2(CCC(Oc3cncc(C(F)(F)F)n3)CC2)C1] | 452.3 [M + H]⁺ |
| 453 | [7-(4-Fluoro-2-methylsulfonyl- phenoxy)-2-azaspiro[3.5]nonan- 2-yl]-[3-(4H-1,2,4-triazol-3- yl)pyrrolidin-1-yl]methanone | 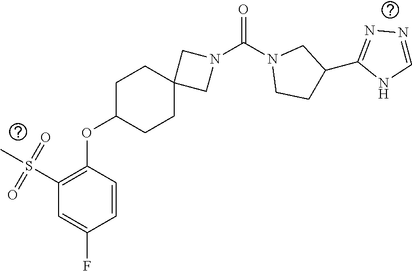 [SMILES: CS(=O)(=O)c1cc(F)ccc1OC1CCC2(CC1)CN(C(=O)N1CCC(c3nnc[nH]3)C1)C2] | 478.3 [M + H]⁺ |
| 455 | [3-(4H-1,2,4-Triazol-3- yl)pyrrolidin-1-yl]-[7-[6- (trifluoromethyl)pyridazin-3- yl]oxy-2-azaspiro[3.5]nonan-2- yl]methanone | 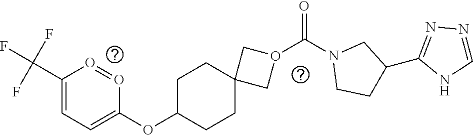 | 452.3 [M + H]⁺ |
| 456 | [3-(4H-1,2,4-Triazol-3- yl)pyrrolidin-1-yl]-[7-[5- (trifluoromethyl)pyrazin-2- yl]oxy-2-azaspiro[3.5]nonan-2- yl]methanone | 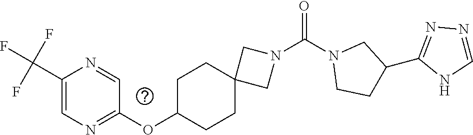 [SMILES: O=C(N1CCC(c2nnc[nH]2)C1)N1CC2(CCC(Oc3cnc(C(F)(F)F)cn3)CC2)C1] | 452.3 [M + H]⁺ |
| 457 | [(3S)-3-(1,2,4-Triazol-4- yl)pyrrolidin-1-yl]-[7-[5- (trifluoromethyl)pyrazin-2- yl]oxy-2-azaspiro[3.5]nonan-2- yl]methanone | 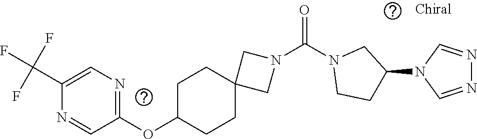 [SMILES: O=C(N1CC[C@H](n2cnnc2)C1)N1CC2(CCC(Oc3cnc(C(F)(F)F)cn3)CC2)C1] | 452.4 [M + H]⁺ |
| 461 | 1-[7-[[2-Fluoro-4- (trifluoromethyl)phenyl]methyl]- 2,7-diazaspiro[3.5]nonane-2- carbonyl]piperidine-3- carboxamide | 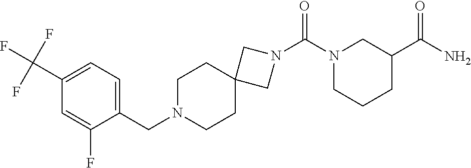 [SMILES: NC(=O)C1CCCN(C(=O)N2CC3(CCN(Cc4ccc(C(F)(F)F)cc4F)CC3)C2)C1] | 457.3 [M + H]⁺ |
| 462 | 1-[7-[[2-Fluoro-4- (trifluoromethyl)phenyl]methyl]- 2,7-diazaspiro[3.5]nonane-2- carbonyl]pyrrolidine-3- sulfonamide | 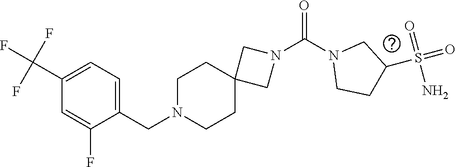 [SMILES: NS(=O)(=O)C1CCN(C(=O)N2CC3(CCN(Cc4ccc(C(F)(F)F)cc4F)CC3)C2)C1] | 479.2 [M + H]⁺ |
| 463 | [6-[(3- Methylsulfonylphenyl)methyl]- 2-azaspiro[3.3]heptan-2-yl]- [(3S)-3-(1H-1,2,4-triazol-5- yl)pyrrolidin-1-yl]methanone | 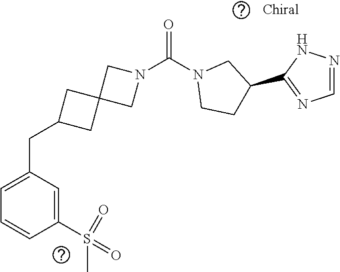 [SMILES: CS(=O)(=O)c1cccc(CC2CC3(C2)CN(C(=O)N2CC[C@H](c4ncn[nH]4)C2)C3)c1] | 430.2 [M + H]⁺ |
| 464 | [6-[(4- Methylsulfonylphenyl)methyl]- 2-azaspiro[3.3]heptan-2-yl]- [(3S)-3-(1H-1,2,4-triazol-5- yl)pyrrolidin-1-yl]methanone | 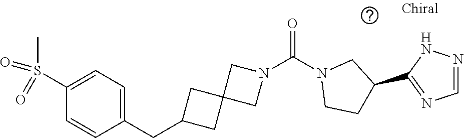 [SMILES: CS(=O)(=O)c1ccc(CC2CC3(C2)CN(C(=O)N2CC[C@H](c4ncn[nH]4)C2)C3)cc1] | 430.2 [M + H]⁺ |
| 465 | (3S)-1-[6-[(3- Methylsulfonylphenyl)methyl]- 2-azaspiro[3.3]heptane-2- carbonyl]pyrrolidine-3- carboxamide | 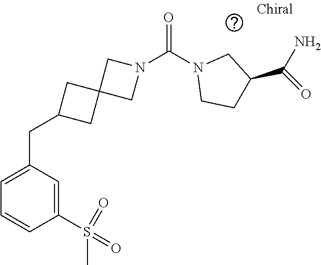 [SMILES: CS(=O)(=O)c1cccc(CC2CC3(C2)CN(C(=O)N2CC[C@H](C(N)=O)C2)C3)c1] | 406.2 [M + H]⁺ |
| 466 | (3S)-1-[6-[(4- Methylsulfonylphenyl)methyl]- 2-azaspiro[3.3]heptane-2- carbonyl]pyrrolidine-3- carboxamide | 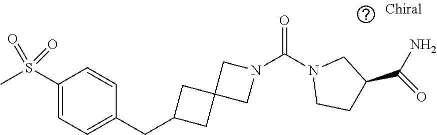 [SMILES: CS(=O)(=O)c1ccc(CC2CC3(C2)CN(C(=O)N2CC[C@H](C(N)=O)C2)C3)cc1] | 406.2 [M + H]⁺ |
| 467 | [(3S)-3-(1H-1,2,4-Triazol-5- yl)pyrrolidin-1-yl]-[3-[6-[[1- (trifluoromethyl)cyclopropyl] methylamino]-3-pyridyl]azetidin- 1-yl]methanone | 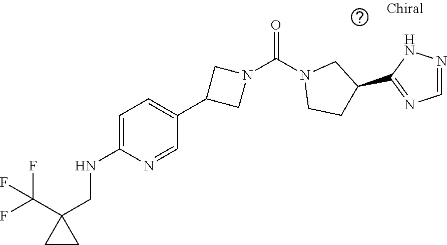 [SMILES: O=C(N1CC(c2ccc(NCC3(C(F)(F)F)CC3)nc2)C1)N1CC[C@H](c2ncn[nH]2)C1] | 436.3 [M + H]⁺ |
| 469 | 4-Fluoro-N-[1-[(3S)-3-(1H- 1,2,4-triazol-5-yl)pyrrolidine-1- carbonyl]-4- piperidyl]benzenesulfonamide | 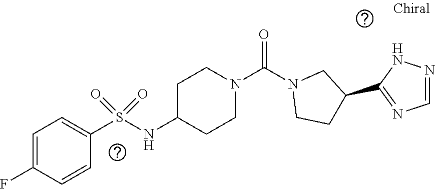 [SMILES: O=C(N1CCC(NS(=O)(=O)c2ccc(F)cc2)CC1)N1CC[C@H](c2ncn[nH]2)C1] | 423.2 [M + H]⁺ |
| 470 | N-[1-[(3S)-3-(1H-1,2,4-Triazol- 5-yl)pyrrolidine-1-carbonyl]-4- piperidyl]-4- (trifluoromethyl) benzenesulfonamide | 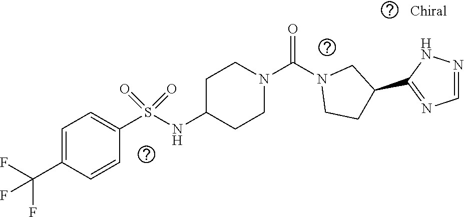 [SMILES: O=C(N1CCC(NS(=O)(=O)c2ccc(C(F)(F)F)cc2)CC1)N1CC[C@H](c2ncn[nH]2)C1] | 473.2 [M + H]⁺ |
| 471 | (3S)-1-[4-[[4- (Trifluoromethyl)phenyl] sulfonylamino] piperidine-1- carbonyl]pyrrolidine-3- carboxamide | 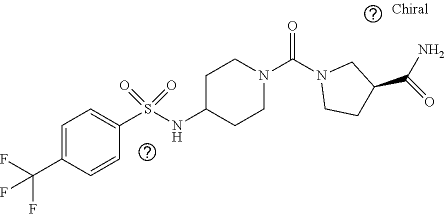 [SMILES: NC(=O)[C@H]1CCN(C(=O)N2CCC(NS(=O)(=O)c3ccc(C(F)(F)F)cc3)CC2)C1] | 449.2 [M + H]⁺ |
| 472 | 4-Chloro-N-[[1-[(3S)-3-(1H- 1,2,4-triazol-5-yl)pyrrolidine-1- carbonyl]-4- piperidyl]methyl] benzenesulfonamide | 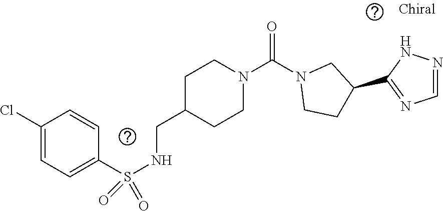 [SMILES: O=C(N1CCC(CNS(=O)(=O)c2ccc(Cl)cc2)CC1)N1CC[C@H](c2ncn[nH]2)C1] | 453.2 [M + H]⁺ |
| 473 | (3S)-1-[7-(4-Fluoro-2- methylsulfonyl-phenoxy)-2- azaspiro[3.5]nonane-2- carbonyl]pyrrolidine-3- carboxamide | 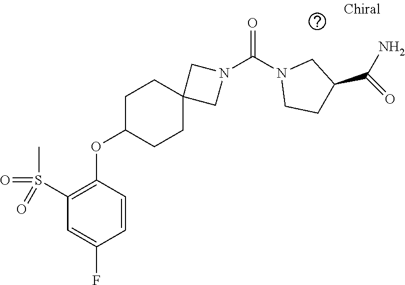 [SMILES: CS(=O)(=O)c1cc(F)ccc1OC1CCC2(CC1)CN(C(=O)N1CC[C@H](C(N)=O)C1)C2] | 454.4 [M + H]⁺ |
| 475 | (3S)-1-[7-[2- (Trifluoromethyl)pyrimidin-4- yl]oxy-2-azaspiro[3.5]nonane-2- carbonyl]pyrrolidine-3- carboxamide | 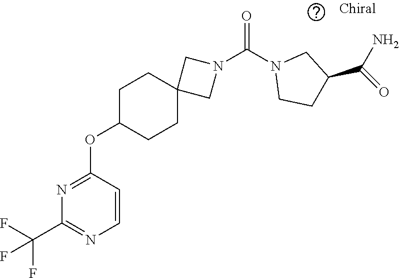 [SMILES: NC(=O)[C@H]1CCN(C(=O)N2CC3(CCC(Oc4ccnc(C(F)(F)F)n4)CC3)C2)C1] | 428.4 [M + H]⁺ |
| 476 | (3S)-1-[7-[[3- (Trifluoromethoxy)phenyl] sulfonylamino]-2- azaspiro[3.5]nonane-2- carbonyl]pyrrolidine-3- carboxamide | 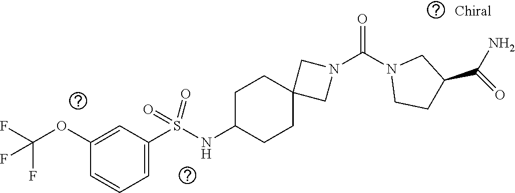 [SMILES: NC(=O)[C@H]1CCN(C(=O)N2CC3(CCC(NS(=O)(=O)c4cccc(OC(F)(F)F)c4)CC3)C2)C1] | 505.3 [M + H]⁺ |
| 477 | N-[2-[(3S)-3-(1H-1,2,4-Triazol- 5-yl)pyrrolidine-1-carbonyl]-2- azaspiro[3.5]nonan-7-yl]-3- (trifluoromethoxy)benzene- sulfonamide | 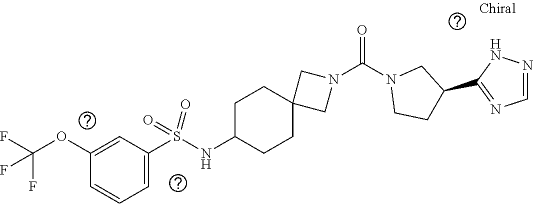 [SMILES: O=C(N1CC[C@H](c2ncn[nH]2)C1)N1CC2(CCC(NS(=O)(=O)c3cccc(OC(F)(F)F)c3)CC2)C1] | 529.4 [M + H]⁺ |
| 479 | (5R)-3-[3-[4-(4-Chloro-2- methylsulfonyl- phenyl)phenyl]azetidine-1- carbonyl]-4,5-dihydroisoxazole- 5-carboxamide | 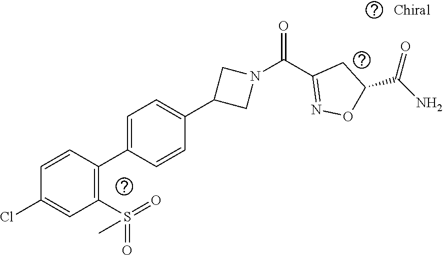 [SMILES: CS(=O)(=O)c1cc(Cl)ccc1-c1ccc(C2CN(C(=O)C3=NO[C@@H](C(N)=O)C3)C2)cc1] | 462.1 [M + H]⁺ |
| 480 | 1-[3-[4-(4-Chloro-2- methylsulfonyl- phenyl)phenyl]azetidine-1- carbonyl]azetidine-3- sulfonamide | 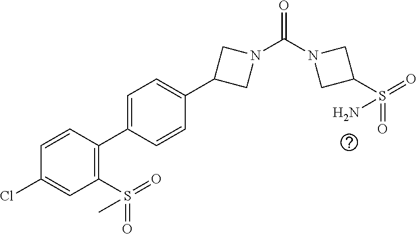 [SMILES: CS(=O)(=O)c1cc(Cl)ccc1-c1ccc(C2CN(C(=O)N3CC(S(N)(=O)=O)C3)C2)cc1] | 484.1 [M + H]⁺ |
| 481 | 1-[3-[4-(4-Chloro-2- methylsulfonyl- phenyl)phenyl]azetidine-1- carbonyl]piperidine-4- sulfonamide | 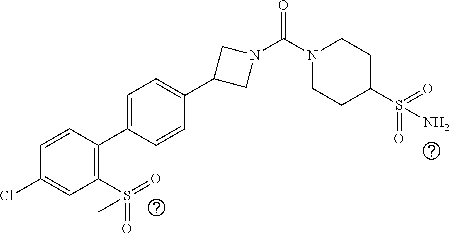 [SMILES: CS(=O)(=O)c1cc(Cl)ccc1-c1ccc(C2CN(C(=O)N3CCC(S(N)(=O)=O)CC3)C2)cc1] | 512.1 [M + H]⁺ |
| 482 | N-[[1-[(3S)-3-(1H-1,2,4-Triazol- 5-yl)pyrrolidine-1-carbonyl]-4- piperidyl]methyl]-4- (trifluoromethyl)benzene- sulfonamide | 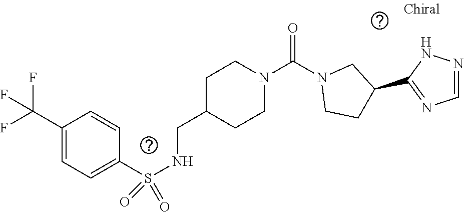 [SMILES: O=C(N1CCC(CNS(=O)(=O)c2ccc(C(F)(F)F)cc2)CC1)N1CC[C@H](c2ncn[nH]2)C1] | 487.3 [M + H]⁺ |
| 483 | (3S)-1-[4-[[[4- (Trifluoromethyl)phenyl]sulfonyl- amino]methyl]piperidine-1- carbonyl]pyrrolidine-3- carboxamide | 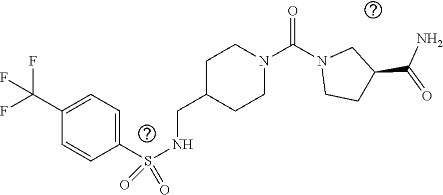 [SMILES: NC(=O)[C@H]1CCN(C(=O)N2CCC(CNS(=O)(=O)c3ccc(C(F)(F)F)cc3)CC2)C1] | 463.3 [M + H]⁺ |
| 484 | N-[[1-[(3S)-3-(1H-1,2,4-Triazol- 5-yl)pyrrolidine-1-carbonyl]-4- piperidyl]methyl]-4- (trifluoromethoxy)benzene- sulfonamide | 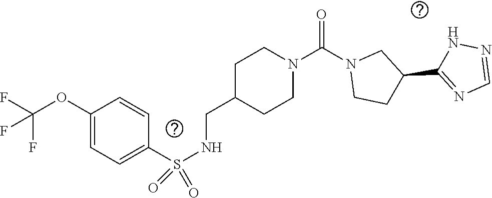 [SMILES: O=C(N1CCC(CNS(=O)(=O)c2ccc(OC(F)(F)F)cc2)CC1)N1CC[C@H](c2ncn[nH]2)C1] | 503.3 [M + H]⁺ |
| 485 | (3S)-1-[4-[[[4- (Trifluoromethoxy)phenyl] sulfonylamino] methyl]piperidine- 1-carbonyl]pyrrolidine-3- carboxamide | 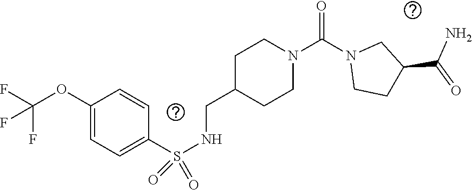 [SMILES: NC(=O)[C@H]1CCN(C(=O)N2CCC(CNS(=O)(=O)c3ccc(OC(F)(F)F)cc3)CC2)C1] | 479.3 [M + H]⁺ |
| 486 | (3S)-1-[3-[4-(6,6-Difluoro-2- azaspiro[3.3]heptan-2- yl)phenyl]azetidine-1- carbonyl]pyrrolidine-3- carboxamide | 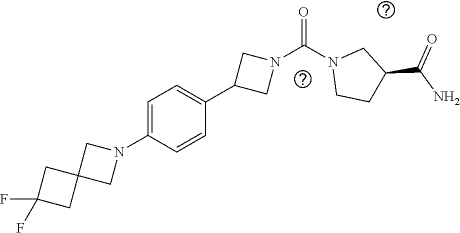 [SMILES: NC(=O)[C@H]1CCN(C(=O)N2CC(c3ccc(N4CC5(C4)CC(F)(F)C5)cc3)C2)C1] | 405.2 [M + H]⁺ |
| 487 | (3S)-1-[3-[4-[3- (Trifluoromethyl)cyclobutyl] phenyl]azetidine-1- carbonyl]pyrrolidine-3- carboxamide | 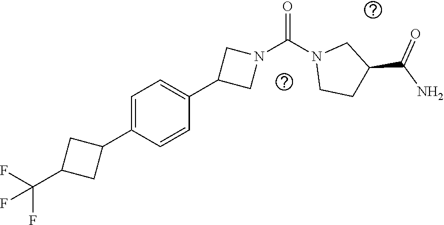 [SMILES: NC(=O)[C@H]1CCN(C(=O)N2CC(c3ccc(C4CC(C(F)(F)F)C4)cc3)C2)C1] | 396.2 [M + H]⁺ |
| 488 | (3S)-1-[3-[4-(2- Azaspiro[3.4]octan-2- yl)phenyl]azetidine-1- carbonyl]pyrrolidine-3- carboxamide | 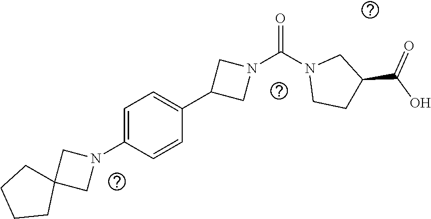 [SMILES: O=C(O)[C@H]1CCN(C(=O)N2CC(c3ccc(N4CC5(CCCC5)C4)cc3)C2)C1] | 383.2 [M + H]⁺ |
| 489 | [(3S)-3-(1H-1,2,4-Triazol-5- yl)pyrrolidin-1-yl]-[6-[[1- (trifluoromethyl)cyclopropyl] methylamino]-2- azaspiro[3.3]heptan-2- yl]methanone | 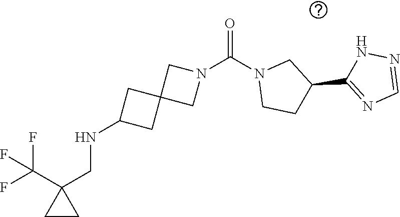 [SMILES: O=C(N1CC[C@H](c2ncn[nH]2)C1)N1CC2(CC(NCC3(C(F)(F)F)CC3)C2)C1] | 399.3 [M + H]⁺ |
| 490 | [6-[(4-Fluoro-2-methylsulfonyl- phenyl)methyl]-2- azaspiro[3.3]heptan-2-yl]-[(3S)- 3-(1H-1,2,4-triazol-5- yl)pyrrolidin-1-yl]methanone | 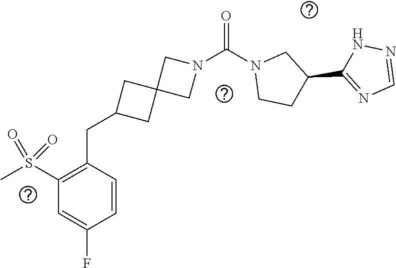 [SMILES: CS(=O)(=O)c1cc(F)ccc1CC1CC2(C1)CN(C(=O)N1CC[C@H](c3ncn[nH]3)C1)C2] | 448.2 [M + H]⁺ |
| 491 | [3-[6-(3-Hydroxy-3-methyl- azetidin-1-yl)-3-pyridyl]azetidin- 1-yl]-[(3S)-3-(1H-1,2,4-triazol- 5-yl)pyrrolidin-1-yl]methanone | 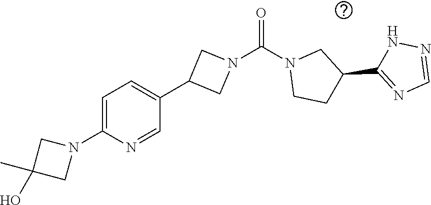 [SMILES: CC1(O)CN(c2ccc(C3CN(C(=O)N4CC[C@H](c5ncn[nH]5)C4)C3)cn2)C1] | 384.3 [M + H]⁺ |
| 492 | [3-[6-[3-Hydroxy-3- (trifluoromethyl)azetidin-1-yl]- 3-pyridyl]azetidin-1-yl]-[(3S)-3- (1H-1,2,4-triazol-5- yl)pyrrolidin-1-yl]methanone | 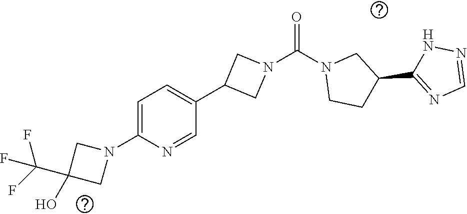 [SMILES: O=C(N1CC(c2ccc(N3CC(O)(C(F)(F)F)C3)nc2)C1)N1CC[C@H](c2ncn[nH]2)C1] | 438.2 [M + H]⁺ |
| 493 | [3-[6-[3-Hydroxy-3- (trifluoromethyl)pyrrolidin-1- yl]-3-pyridyl]azetidin-1-yl]-[rac- (3S)-3-(1H-1,2,4-triazol-5- yl)pyrrolidin-1-yl]methanone | 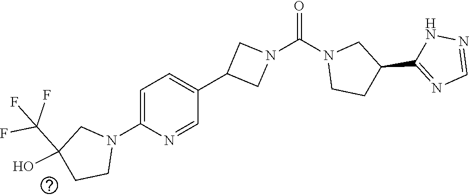 [SMILES: O=C(N1CC(c2ccc(N3CCC(O)(C(F)(F)F)C3)nc2)C1)N1CC[C@H](c2ncn[nH]2)C1] | 452.3 [M + H]⁺ |
| 494 | [3-[4-[1-(1H-Tetrazol-5- yl)cyclopropyl]phenyl]azetidin- 1-yl]-[(3S)-3-(1H-1,2,4-triazol- 5-yl)pyrrolidin-1-yl]methanone | 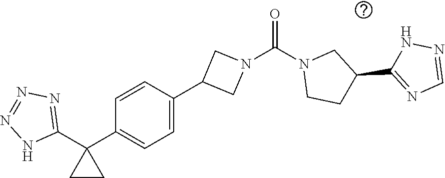 [SMILES: O=C(N1CC(c2ccc(C3(c4nnn[nH]4)CC3)cc2)C1)N1CC[C@H](c2ncn[nH]2)C1] | 406.3 [M + H]⁺ |
| 495 | [3-[4-[1-(2-tert-Butyltetrazol-5- yl)cyclopropyl]phenyl]azetidin- 1-yl]-[(3S)-3-(1H-1,2,4-triazol- 5-yl)pyrrolidin-1-yl]methanone | 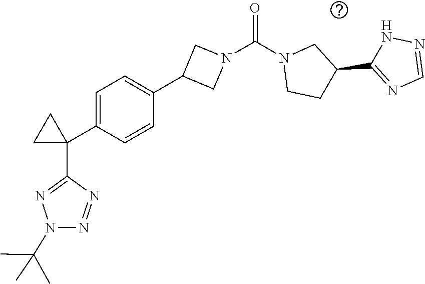 [SMILES: CC(C)(C)n1nnc(C2(c3ccc(C4CN(C(=O)N5CC[C@H](c6ncn[nH]6)C5)C4)cc3)CC2)n1] | 462.3 [M + H]⁺ |
| 496 | (3S)-1-[6-[[1- (Trifluoromethyl)cyclopropyl] methylamino]-2- azaspiro[3.3]heptane-2- carbonyl]pyrrolidine-3- carboxamide | 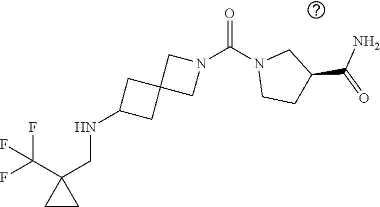 [SMILES: NC(=O)[C@H]1CCN(C(=O)N2CC3(CC(NCC4(C(F)(F)F)CC4)C3)C2)C1] | 375.2 [M + H]⁺ |
| 497 | (3S)-1-[6-[(4-Fluoro-2- methylsulfonyl-phenyl)methyl]- 2-azaspiro[3.3]heptane-2- carbonyl]pyrrolidine-3- carboxamide | 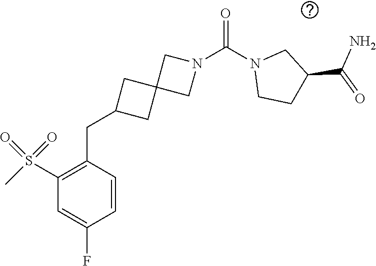 [SMILES: CS(=O)(=O)c1cc(F)ccc1CC1CC2(C1)CN(C(=O)N1CC[C@H](C(N)=O)C1)C2] | 424.3 [M + H]⁺ |
| 498 | (3S)-1-[3-[6-[3-Hydroxy-3- (trifluoromethyl)azetidin-1-yl]- 3-pyridyl]azetidine-1- carbonyl]pyrrolidine-3- carboxamide | 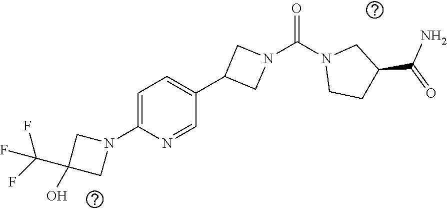 [SMILES: NC(=O)[C@H]1CCN(C(=O)N2CC(c3ccc(N4CC(O)(C(F)(F)F)C4)nc3)C2)C1] | 414.2 [M + H]⁺ |
| 499 | rac-(3S)-1-[3-[6-[3-Hydroxy-3- (trifluoromethyl)pyrrolidin-1- yl]-3-pyridyl]azetidine-1- carbonyl]pyrrolidine-3- carboxamide | 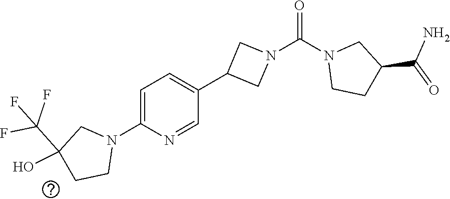 [SMILES: NC(=O)[C@H]1CCN(C(=O)N2CC(c3ccc(N4CCC(O)(C(F)(F)F)C4)nc3)C2)C1] | 428.2 [M + H]⁺ |
| 500 | (3S)-1-[3-[4-[1-(2-tert- Butyltetrazol-5- yl)cyclopropyl]phenyl]azetidine- 1-carbonyl]pyrrolidine-3- carboxamide | 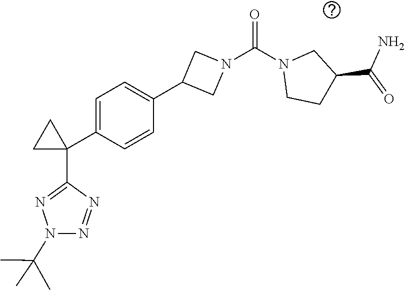 [SMILES: CC(C)(C)n1nnc(C2(c3ccc(C4CN(C(=O)N5CC[C@H](C(N)=O)C5)C4)cc3)CC2)n1] | 438.3 [M + H]⁺ |
| 501 | (3S)-1-[6-[[1- (Trifluoromethyl)cyclopropyl] amino]-2-azaspiro[3.3]heptane- 2-carbonyl]pyrrolidine-3- carboxamide | 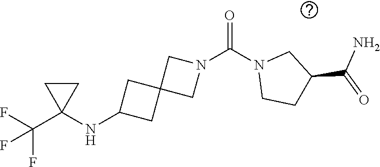 [SMILES: NC(=O)[C@H]1CCN(C(=O)N2CC3(CC(NC4(C(F)(F)F)CC4)C3)C2)C1] | 361.2 [M + H]⁺ |
| 502 | [(3S)-3-(1H-1,2,4-Triazol-5- yl)pyrrolidin-1-yl]-[6-[[1- (trifluoromethyl)cyclopropyl] amino]-2-azaspiro[3.3]heptan- 2-yl]methanone | 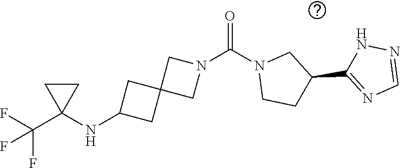 [SMILES: O=C(N1CC[C@H](c2ncn[nH]2)C1)N1CC2(CC(NC3(C(F)(F)F)CC3)C2)C1] | 385.3 [M + H]⁺ |
| 503 | (3S)-1-[3-[6-[(3R)-3-Hydroxy-3- (trifluoromethyl)pyrrolidin-1- yl]-3-pyridyl]azetidine-1- carbonyl]pyrrolidine-3- carboxamide | 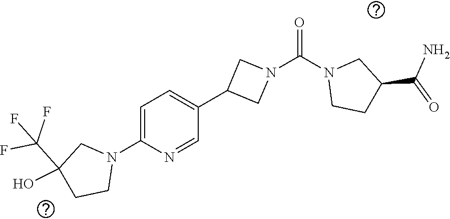 [SMILES: NC(=O)[C@H]1CCN(C(=O)N2CC(c3ccc(N4CCC(O)(C(F)(F)F)C4)nc3)C2)C1] | 428.2 [M + H]⁺ |
| 504 | (3S)-1-[3-[6-[(3S)-3-Hydroxy-3- (trifluoromethyl)pyrrolidin-1- yl]-3-pyridyl]azetidine-1- carbonyl]pyrrolidine-3- carboxamide | 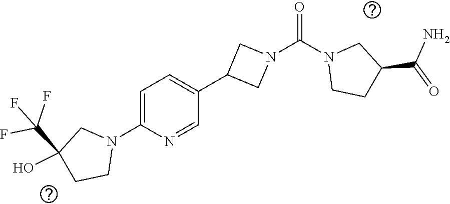 [SMILES: NC(=O)[C@H]1CCN(C(=O)N2CC(c3ccc(N4CC[C@@](O)(C(F)(F)F)C4)nc3)C2)C1] | 428.2 [M + H]⁺ |
| 505 | [3-[4-(4- Fluorophenoxy)phenyl]azetidin- 1-yl]-[(3R)-3-(tetrazol-2- yl)pyrrolidin-1-yl]methanone | 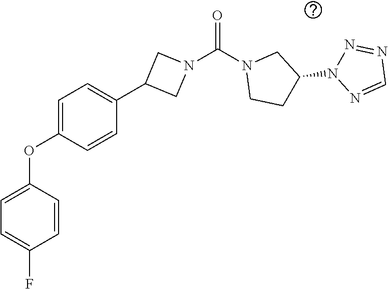 [SMILES: O=C(N1CC(c2ccc(Oc3ccc(F)cc3)cc2)C1)N1CC[C@@H](n2ncnn2)C1] | 409.2 [M + H]⁺ |
| 506 | [(3R)-3-(Tetrazol-2- yl)pyrrolidin-1-yl]-[3-[4-[1- (trifluoromethyl)cyclopropyl] phenyl]azetidin-1-yl]methanone | 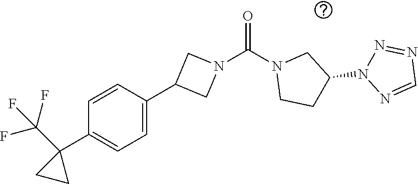 [SMILES: O=C(N1CC(c2ccc(C3(C(F)(F)F)CC3)cc2)C1)N1CC[C@@H](n2ncnn2)C1] | 407.2 [M + H]⁺ |
| 507 | [3-[6-[(3R)-3-Hydroxy-3- (trifluoromethyl)pyrrolidin-1- yl]-3-pyridyl]azetidin-1-yl]- [(3S)-3-(1H-1,2,4-triazol-5- yl)pyrrolidin-1-yl]methanone | 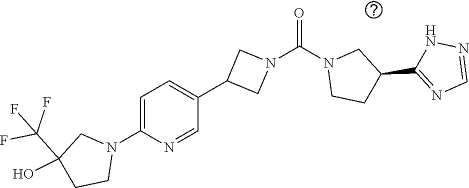 [SMILES: O=C(N1CC(c2ccc(N3CCC(O)(C(F)(F)F)C3)nc2)C1)N1CC[C@H](c2ncn[nH]2)C1] | 452.3 [M + H]⁺ |
| 508 | [3-[6-[(3S)-3-Hydroxy-3- (trifluoromethyl)pyrrolidin-1- yl]-3-pyridyl]azetidin-1-yl]- [(3S)-3-(1H-1,2,4-triazol-5- yl)pyrrolidin-1-yl]methanone | 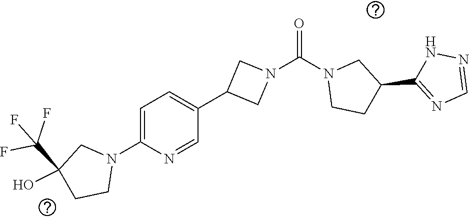 [SMILES: O=C(N1CC(c2ccc(N3CC[C@@](O)(C(F)(F)F)C3)nc2)C1)N1CC[C@H](c2ncn[nH]2)C1] | 452.3 [M + H]⁺ |
| 509 | [3-[6-(4-Chloro-2- methylsulfonyl-phenoxy)-3- pyridyl]azetidin-1-yl]-[(3S)-3- (1,2,4-triazol-4-yl)pyrrolidin-1- yl]methanone | 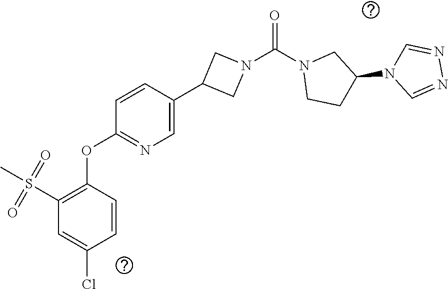 [SMILES: CS(=O)(=O)c1cc(Cl)ccc1Oc1ccc(C2CN(C(=O)N3CC[C@H](n4cnnc4)C3)C2)cn1] | 503.2 [M + H]⁺ |
| 510 | 1-[3-[4-[1- (Trifluoromethyl)cyclopropyl] phenyl]azetidine-1- carbonyl]pyrrolidine-3- sulfonamide | 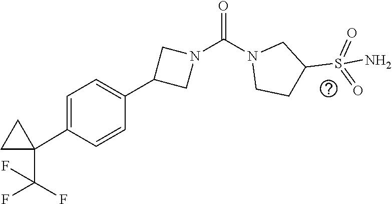 [SMILES: NS(=O)(=O)C1CCN(C(=O)N2CC(c3ccc(C4(C(F)(F)F)CC4)cc3)C2)C1] | 418.2 [M + H]⁺ |
| 511 | 1-[3-[[2-Fluoro-4- (trifluoromethyl)phenyl] methoxy]azetidine-1- carbonyl]pyrrolidine-3- sulfonamide | 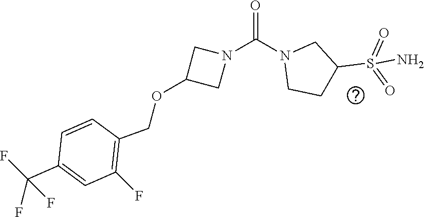 [SMILES: NS(=O)(=O)C1CCN(C(=O)N2CC(OCc3ccc(C(F)(F)F)cc3F)C2)C1] | 426.2 [M + H]⁺ |
| 512 | [(3S)-3-(1H-1,2,4-Triazol-5- yl)pyrrolidin-1-yl]-[3-[[4- (trifluoromethylsulfonyl)phenyl] methoxy]azetidin-1- yl]methanone | 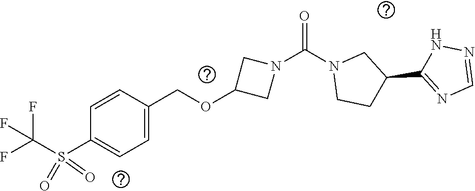 [SMILES: O=C(N1CC(OCc2ccc(S(=O)(=O)C(F)(F)F)cc2)C1)N1CC[C@H](c2ncn[nH]2)C1] | 460.2 [M + H]⁺ |
| 513 | [(3S)-3-(1H-1,2,4-Triazol-5- yl)pyrrolidin-1-yl]-[3-[[3- (trifluoromethylsulfonyl)phenyl] methoxy]azetidin-1- yl]methanone | 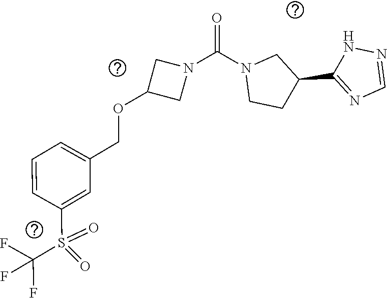 [SMILES: O=C(N1CC(OCc2cccc(S(=O)(=O)C(F)(F)F)c2)C1)N1CC[C@H](c2ncn[nH]2)C1] | 460.2 [M + H]⁺ |
| 514 | Methyl 2-[4-[1-[(3S)-3-(1H- 1,2,4-triazol-5-yl)pyrrolidine-1- carbonyl]azetidin-3- yl]phenyl]benzoate | 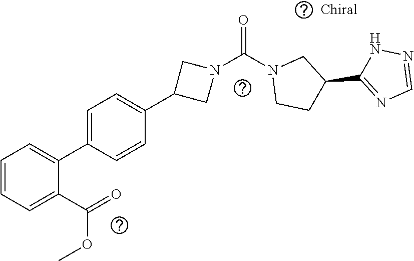 [SMILES: COC(=O)c1ccccc1-c1ccc(C2CN(C(=O)N3CC[C@H](c4ncn[nH]4)C3)C2)cc1] | 432.3 [M + H]⁺ |
| 515 | 1-[4-[1-[rac-(3S)-3-(1H-1,2,4- Triazol-5-yl)pyrrolidine-1- carbonyl]azetidin-3- yl]phenyl]piperidine-2- carboxamide | 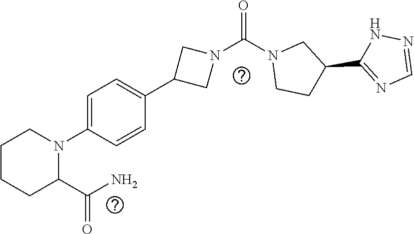 [SMILES: NC(=O)C1CCCCN1c1ccc(C2CN(C(=O)N3CC[C@H](c4ncn[nH]4)C3)C2)cc1] | 424.3 [M + H]⁺ |
| 516 | [3-[[2-Fluoro-5- (trifluoromethyl)phenyl]methyl- amino]azetidin-1-yl]-[(3S)-3- (1H-1,2,4-triazol-5-yl) pyrrolidin-1- yl]methanone | 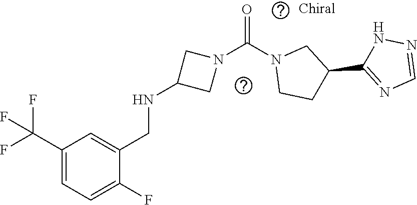 [SMILES: O=C(N1CC(NCc2cc(C(F)(F)F)ccc2F)C1)N1CC[C@H](c2ncn[nH]2)C1] | 413.3 [M + H]⁺ |
| 517 | [(3S)-3-(1H-1,2,4-Triazol-5- yl)pyrrolidin-1-yl]-[3-[[4- (trifluoromethyl)phenyl]methyl- amino]azetidin-1-yl]methanone | 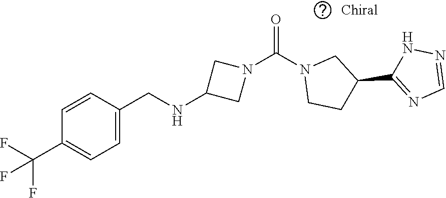 [SMILES: O=C(N1CC(NCc2ccc(C(F)(F)F)cc2)C1)N1CC[C@H](c2ncn[nH]2)C1] | 395.3 [M + H]⁺ |
| 518 | [(3S)-3-(Tetrazol-1- yl)pyrrolidin-1-yl]-[3-[2-[1- (trifluoromethyl)cyclopropyl] pyrimidin-5-yl]azetidin-1- yl]methanone | 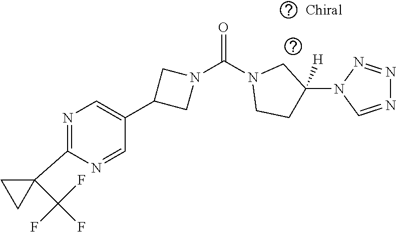 [SMILES: O=C(N1CC(c2cnc(C3(C(F)(F)F)CC3)nc2)C1)N1CC[C@H](n2cnnn2)C1] | 409.2 [M + H]⁺ |
| 519 | (3S)-1-[7-[[5- (Trifluoromethyl)pyrazin-2- yl]amino]-2- azaspiro[3.5]nonane-2- carbonyl]pyrrolidine-3- carboxamide | 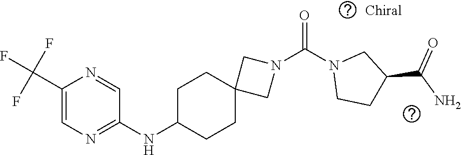 [SMILES: NC(=O)[C@H]1CCN(C(=O)N2CC3(CCC(Nc4cnc(C(F)(F)F)cn4)CC3)C2)C1] | 427.4 [M + H]⁺ |
| 520 | (3R)-1-[7-[[5- (Trifluoromethyl)pyrazin-2- yl]amino]-2- azaspiro[3.5]nonane-2- carbonyl]pyrrolidine-3- carboxamide | 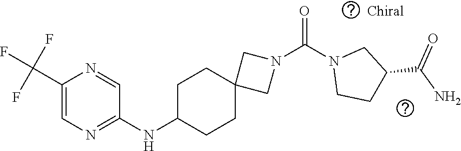 [SMILES: NC(=O)[C@@H]1CCN(C(=O)N2CC3(CCC(Nc4cnc(C(F)(F)F)cn4)CC3)C2)C1] | 427.4 [M + H]⁺ |
| 522 | [3-[4-(4- Fluorophenoxy)phenyl]azetidin- 1-yl]-[(3S)-3-(tetrazol-2- yl)pyrrolidin-1-yl]methanone | 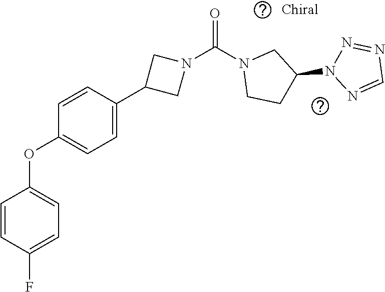 [SMILES: O=C(N1CC(c2ccc(Oc3ccc(F)cc3)cc2)C1)N1CC[C@H](n2ncnn2)C1] | 409.2 [M + H]⁺ |
| 523 | [3-[4-(4-Chloro-2- methylsulfonyl- phenyl)phenyl]azetidin-1-yl]- [(3S)-3-(tetrazol-2-yl)pyrrolidin- 1-yl]methanone | 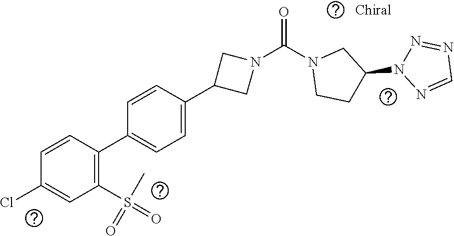 [SMILES: CS(=O)(=O)c1cc(Cl)ccc1-c1ccc(C2CN(C(=O)N3CC[C@H](n4ncnn4)C3)C2)cc1] | 487.2 [M + H]⁺ |
| 524 | [3-[4-(4-Chloro-2- methylsulfonyl- phenyl)phenyl]azetidin-1-yl]- [(3R)-3-(tetrazol-2-yl)pyrrolidin- 1-yl]methanone | 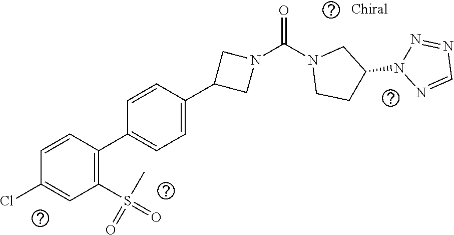 [SMILES: CS(=O)(=O)c1cc(Cl)ccc1-c1ccc(C2CN(C(=O)N3CC[C@@H](n4ncnn4)C3)C2)cc1] | 487.2 [M + H]⁺ |
| 525 | [(3S)-3-(Tetrazol-1- yl)pyrrolidin-1-yl]-[3-[4-[[1- (trifluoromethyl)cyclopropyl] methoxy]phenyl]azetidin-1- yl]methanone | 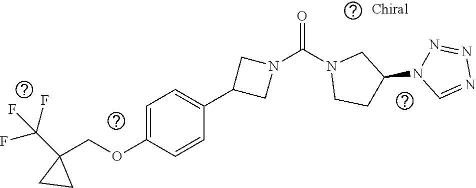 [SMILES: O=C(N1CC(c2ccc(OCC3(C(F)(F)F)CC3)cc2)C1)N1CC[C@H](n2cnnn2)C1] | 437.2 [M + H]⁺ |
| 526 | [(3S)-3-(Tetrazol-1- yl)pyrrolidin-1-yl]-[3-[4-[5- (trifluoromethyl)pyrazin-2- yl]oxyphenyl]azetidin-1- yl]methanone | 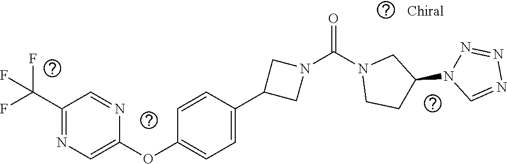 [SMILES: O=C(N1CC(c2ccc(Oc3cnc(C(F)(F)F)cn3)cc2)C1)N1CC[C@H](n2cnnn2)C1] | 461.2 [M + H]⁺ |
| 527 | [(3S)-3-(1H-1,2,4-Triazol-5- yl)pyrrolidin-1-yl]-[3-[6-[[1- (trifluoromethyl)cyclopropyl] amino]-3-pyridyl]azetidin-1- yl]methanone | 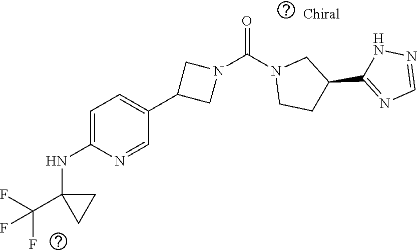 [SMILES: O=C(N1CC(c2ccc(NC3(C(F)(F)F)CC3)nc2)C1)N1CC[C@H](c2ncn[nH]2)C1] | 422.3 [M + H]⁺ |
| 528 | 4,4-Difluoro-1-[4-[1-[rac-(3S)- 3-(1H-1,2,4-triazol-5- yl)pyrrolidine-1- carbonyl]azetidin-3- yl]phenyl]piperidine-2- carboxamide | 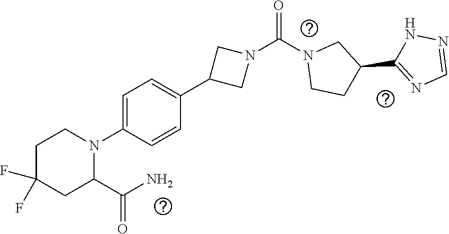 [SMILES: NC(=O)C1CC(F)(F)CCN1c1ccc(C2CN(C(=O)N3CC[C@H](c4ncn[nH]4)C3)C2)cc1] | 460.3 [M + H]⁺ |
| 530 | [3-[[5-Chloro-2- (trifluoromethyl) phenyl]methylamino] azetidin-1-yl]-[(3S)-3-(1H- 1,2,4-triazol-5-yl)pyrrolidin-1- yl]methanone | 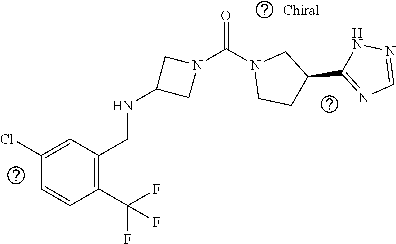 [SMILES: O=C(N1CC(NCc2cc(Cl)ccc2C(F)(F)F)C1)N1CC[C@H](c2ncn[nH]2)C1] | 429.2 [M + H]⁺ |
| 531 | [3-[[3-Chloro-5- (trifluoromethyl) phenyl]methylamino] azetidin-1-yl]-[(3S)-3-(1H- 1,2,4-triazol-5-yl)pyrrolidin-1- yl]methanone | 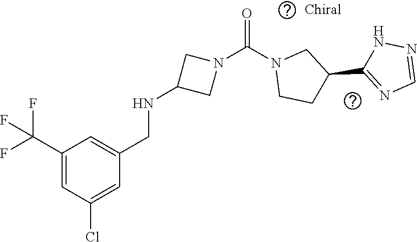 [SMILES: O=C(N1CC(NCc2cc(Cl)cc(C(F)(F)F)c2)C1)N1CC[C@H](c2ncn[nH]2)C1] | 429.2 [M + H]⁺ |
| 532 | [3-[[2-Fluoro-4-(trifluoro- methoxy)phenyl]methyl- amino]azetidin-1-yl]-[(3S)-3- (1H-1,2,4-triazol-5- yl)pyrrolidin-1-yl]methanone | 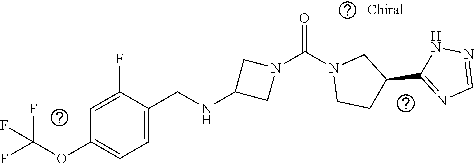 [SMILES: O=C(N1CC(NCc2ccc(OC(F)(F)F)cc2F)C1)N1CC[C@H](c2ncn[nH]2)C1] | 429.2 [M + H]⁺ |
| 533 | [3-[[3-Fluoro-4- (trifluoromethyl)phenyl]methyl- amino]azetidin-1- yl]-[(3S)-3-(1H- 1,2,4-triazol-5-yl)pyrrolidin-1- yl]methanone | 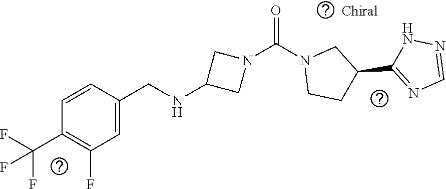 [SMILES: O=C(N1CC(NCc2ccc(C(F)(F)F)c(F)c2)C1)N1CC[C@H](c2ncn[nH]2)C1] | 413.2 [M + H]⁺ |
| 535 | 2-[4-[1-[(3S)-3-(1H-1,2,4- Triazol-5-yl)pyrrolidine-1- carbonyl]azetidin-3- yl]phenyl]benzoic acid | 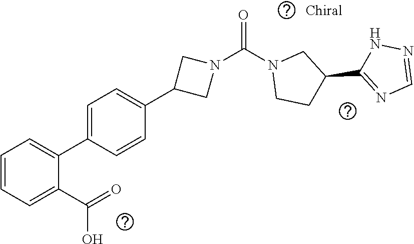 [SMILES: O=C(O)c1ccccc1-c1ccc(C2CN(C(=O)N3CC[C@H](c4ncn[nH]4)C3)C2)cc1] | 418.2 [M + H]⁺ |
| 536 | (3S)-1-[3-[4-(3,5- Dimethylpyrazol-1- yl)phenyl]azetidine-1- carbonyl]pyrrolidine-3- carboxamide | 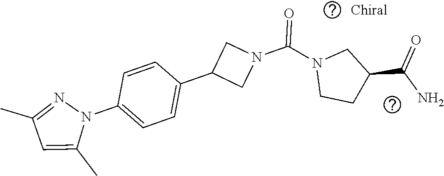 [SMILES: Cc1cc(C)n(-c2ccc(C3CN(C(=O)N4CC[C@H](C(N)=O)C4)C3)cc2)n1] | 368.2 [M + H]⁺ |
| 537 | [6-[(3- Methylsulfonylphenyl)methyl]- 2-azaspiro[3.3]heptan-2-yl]- [(3S)-3-(tetrazol-1-yl)pyrrolidin- 1-yl]methanone | 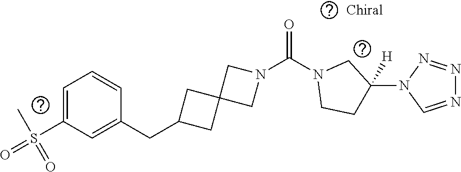 [SMILES: CS(=O)(=O)c1cccc(CC2CC3(C2)CN(C(=O)N2CC[C@H](n4cnnn4)C2)C3)c1] | 431.2 [M + H]⁺ |
| 538 | [3-[[2-Chloro-4- (trifluoromethyl)phenyl]methyl- amino]azetidin-1-yl]-[(3S)-3- (tetrazol-1-yl)pyrrolidin-1- yl]methanone | 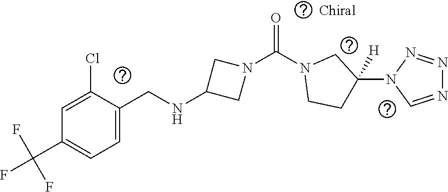 [SMILES: O=C(N1CC(NCc2ccc(C(F)(F)F)cc2Cl)C1)N1CC[C@H](n2cnnn2)C1] | 430.1 [M + H^{]+} |
| 539 | [3-[[3-Fluoro-4- (trifluoromethyl)phenyl]methyl- amino]azetidin-1-yl]-[(3S)-3- (tetrazol-1-yl)pyrrolidin-1- yl]methanone | 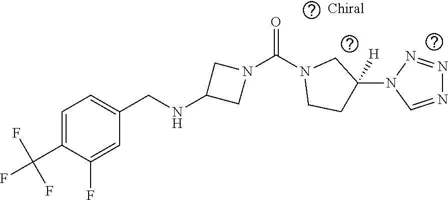 [SMILES: O=C(N1CC(NCc2ccc(C(F)(F)F)c(F)c2)C1)N1CC[C@H](n2cnnn2)C1] | 414.2 [M + H]⁺ |
| 540 | [3-[[2-Fluoro-4- (trifluoromethoxy)phenyl]methyl- amino]azetidin-1-yl]-[(3S)-3- (tetrazol-1-yl)pyrrolidin-1- yl]methanone | 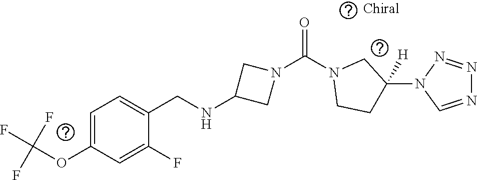 [SMILES: O=C(N1CC(NCc2ccc(OC(F)(F)F)cc2F)C1)N1CC[C@H](n2cnnn2)C1] | 430.2 [M + H]⁺ |
| 541 | [3-[[5-Chloro-2- (trifluoromethyl)phenyl]methyl- amino]azetidin-1-yl]-[(3S)-3- (tetrazol-1-yl)pyrrolidin-1- yl]methanone | 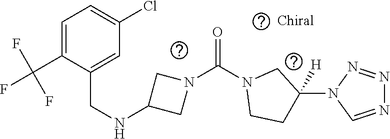 [SMILES: O=C(N1CC(NCc2cc(Cl)ccc2C(F)(F)F)C1)N1CC[C@H](n2cnnn2)C1] | 430.1 [M + H]⁺ |
| 542 | [3-[[3-Chloro-5- (trifluoromethyl)phenyl]methyl- amino]azetidin-1-yl]-[(3S)-3- (tetrazol-1-yl)pyrrolidin-1- yl]methanone | 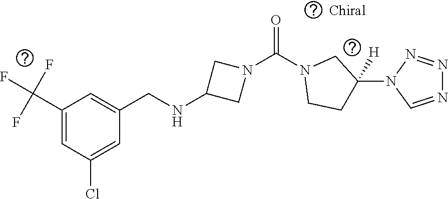 [SMILES: O=C(N1CC(NCc2cc(Cl)cc(C(F)(F)F)c2)C1)N1CC[C@H](n2cnnn2)C1] | 430.2 [M + H]⁺ |
| 543 | [(3S)-3-(Tetrazol-1- yl)pyrrolidin-1-yl]-[3-[5-[1- (trifluoromethyl)cyclopropyl]-2- pyridyl]azetidin-1-yl]methanone | 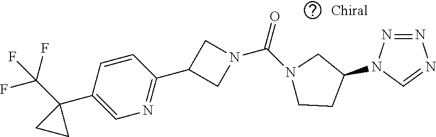 [SMILES: O=C(N1CC(c2ccc(C3(C(F)(F)F)CC3)cn2)C1)N1CC[C@H](n2cnnn2)C1] | 408.2 [M + H]⁺ |
| 545 | 1-[3-[6-(2-Azaspiro[3.4]octan-2- yl)-3-pyridyl]azetidine-1- carbonyl]pyrrolidine-3- sulfonamide | 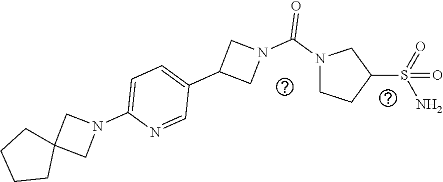 [SMILES: NS(=O)(=O)C1CCN(C(=O)N2CC(c3ccc(N4CC5(CCCC5)C4)nc3)C2)C1] | 420.2 [M + H]⁺ |
| 546 | 1-[4-[1-[(3S)-3-(1H-1,2,4- Triazol-5-yl)pyrrolidine-1- carbonyl]azetidin-3- yl]phenyl]piperidine-2- carboxamide | 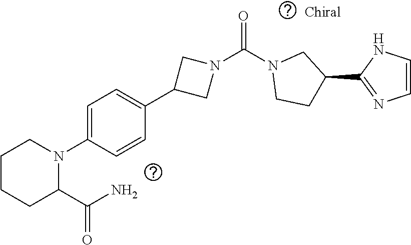 [SMILES: NC(=O)C1CCCCN1c1ccc(C2CN(C(=O)N3CC[C@H](c4ncc[nH]4)C3)C2)cc1] | 424.3 [M + H]⁺ |
| 547 | 1-[4-[1-[(3S)-3-(1H-1,2,4- Triazol-5-yl)pyrrolidine-1- carbonyl]azetidin-3- yl]phenyl]piperidine-2- carboxamide | 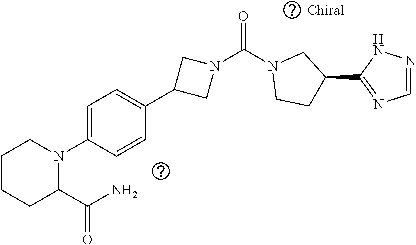 [SMILES: NC(=O)C1CCCCN1c1ccc(C2CN(C(=O)N3CC[C@H](c4ncn[nH]4)C3)C2)cc1] | 424.3 [M + H]⁺ |
| 549 | 2-[4-[1-[(3S)-3-(1H-1,2,4- Triazol-5-yl)pyrrolidine-1- carbonyl]azetidin-3- yl]phenyl]benzamide | 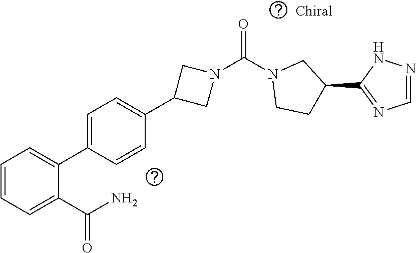 [SMILES: NC(=O)c1ccccc1-c1ccc(C2CN(C(=O)N3CC[C@H](c4ncn[nH]4)C3)C2)cc1] | 417.3 [M + H]⁺ |
| 551 | (3S)-1-[3-[[4- (Trifluoromethylsulfonyl) phenyl]methoxy]azetidine-1- carbonyl]pyrrolidine-3- carboxamide | 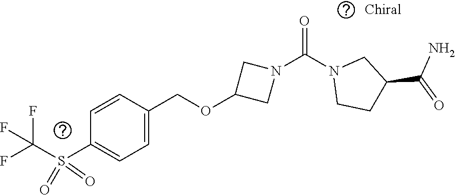 [SMILES: NC(=O)[C@H]1CCN(C(=O)N2CC(OCc3ccc(S(=O)(=O)C(F)(F)F)cc3)C2)C1] | 436.2 [M + H]⁺ |
| 552 | (3S)-1-[3-[[3- (Trifluoromethylsulfonyl) phenyl]methoxy]azetidine-1- carbonyl]pyrrolidine-3- carboxamide | 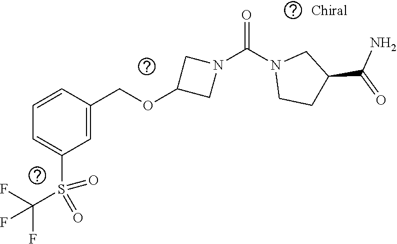 [SMILES: NC(=O)[C@H]1CCN(C(=O)N2CC(OCc3cccc(S(=O)(=O)C(F)(F)F)c3)C2)C1] | 436.2 [M + H]⁺ |
| 553 | (3S)-1-[3-[5-[6- (Trifluoromethyl)-3-pyridyl]- 1,2,4-oxadiazol-3-yl]azetidine-1- carbonyl]pyrrolidine-3- carboxamide | 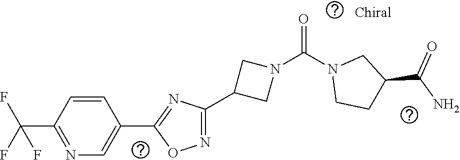 [SMILES: NC(=O)[C@H]1CCN(C(=O)N2CC(c3noc(-c4ccc(C(F)(F)F)nc4)n3)C2)C1] | 411.2 [M + H]⁺ |
| 554 | (3S)-1-[3-[5-[3- (Trifluoromethyl)-1- bicyclo[1.1.1]pentanyl]-1,2,4- oxadiazol-3-yl]azetidine-1- carbonyl]pyrrolidine-3- carboxamide | 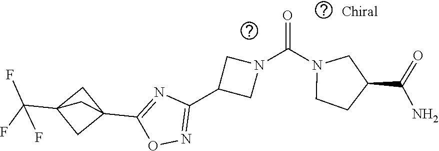 [SMILES: NC(=O)[C@H]1CCN(C(=O)N2CC(c3noc(C45CC(C(F)(F)F)(C4)C5)n3)C2)C1] | 400.2 [M + H]⁺ |
| 555 | rac-(3S)-1-[3-[6-[3- (Trifluoromethyl)pyrrolidin-1- yl]-3-pyridyl]azetidine-1- carbonyl]pyrrolidine-3- carboxamide | 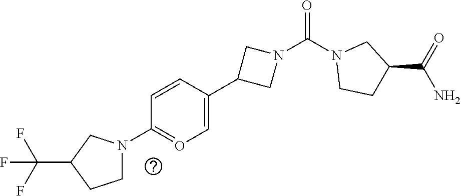 | 412.3 [M + H]⁺ |
| 556 | N-Ethyl-2-[4-[1-[(3S)-3-(1H- 1,2,4-triazol-5-yl)pyrrolidine-1- carbonyl]azetidin-3- yl]phenyl]benzamide | 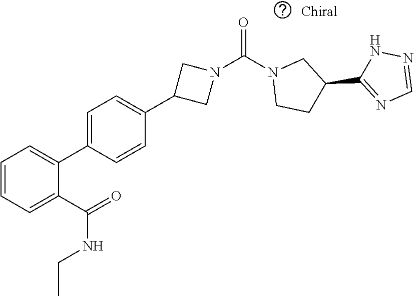 [SMILES: CCNC(=O)c1ccccc1-c1ccc(C2CN(C(=O)N3CC[C@H](c4ncn[nH]4)C3)C2)cc1] | 445.3 [M + H]⁺ |
| 557 | [3-[Cyclopropylmethyl-[[2- fluoro-4- (trifluoromethyl)phenyl]methyl] amino]azetidin-1-yl]-[(3S)-3- (tetrazol-1-yl)pyrrolidin-1- yl]methanone | 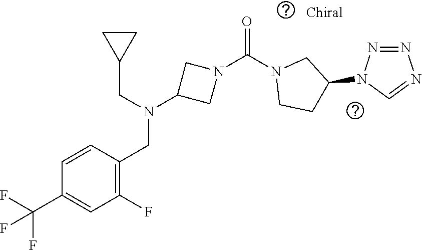 [SMILES: O=C(N1CC(N(Cc2ccc(C(F)(F)F)cc2F)CC2CC2)C1)N1CC[C@H](n2cnnn2)C1] | 468.2 [M + H]⁺ |
| 560 | 1-[3-[4-(4-Chloro-2- methylsulfonyl- phenyl)phenyl]azetidine-1- carbonyl]azetidine-3- carboxamide | 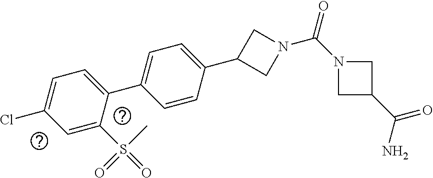 [SMILES: CS(=O)(=O)c1cc(Cl)ccc1-c1ccc(C2CN(C(=O)N3CC(C(N)=O)C3)C2)cc1] | 448.1 [M + H]⁺ |
| 561 | 1-[3-[4-[5- (Trifluoromethyl)pyrimidin-2- yl]oxyphenyl]azetidine-1- carbonyl]azetidine-3- carboxamide | 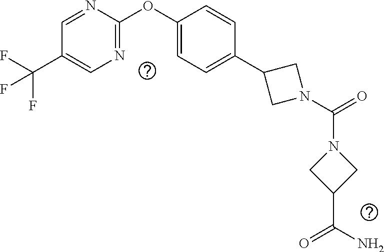 [SMILES: NC(=O)C1CN(C(=O)N2CC(c3ccc(Oc4ncc(C(F)(F)F)cn4)cc3)C2)C1] | 422.1 [M + H]⁺ |
| 562 | [3-[[2-Fluoro-4- (trifluoromethyl)phenyl]methyl- methyl-amino]azetidin-1-yl]- [(3S)-3-(tetrazol-1-yl)pyrrolidin- 1-yl]methanone | 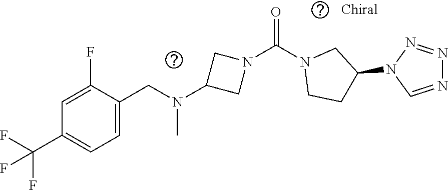 [SMILES: CN(Cc1ccc(C(F)(F)F)cc1F)C1CN(C(=O)N2CC[C@H](n3cnnn3)C2)C1] | 428.2 [M + H]⁺ |
| 563 | (3S)-1-[3-(5-Spiro[3.3]heptan-2- ylpyrazin-2-yl)azetidine-1- carbonyl]pyrrolidine-3- carboxamide | 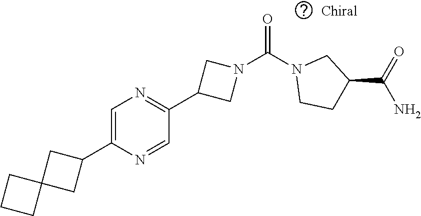 [SMILES: NC(=O)[C@H]1CCN(C(=O)N2CC(c3cnc(C4CC5(CCC5)C4)cn3)C2)C1] | 370.2 [M + H]⁺ |
| 564 | (3S)-1-[3-[4-[4-Methyl-2- (pyrrolidine-1-carbonyl) phenyl]phenyl]azetidine- 1-carbonyl]pyrrolidine-3- carboxamide | 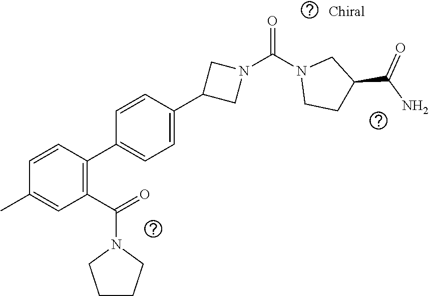 [SMILES: Cc1ccc(-c2ccc(C3CN(C(=O)N4CC[C@H](C(N)=O)C4)C3)cc2)c(C(=O)N2CCCC2)c1] | 461.3 [M + H]⁺ |
| 566 | [3-[4-(4-Chloro-2- methylsulfonyl- phenyl)phenyl]azetidin-1-yl]-[3- (methylsulfonimidoyl)pyrrolidin- 1-yl]methanone | 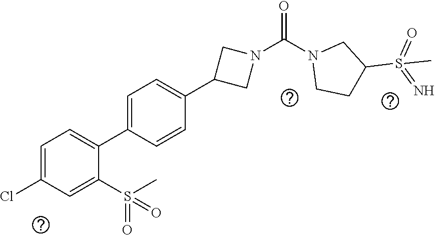 [SMILES: CS(=N)(=O)C1CCN(C(=O)N2CC(c3ccc(-c4ccc(Cl)cc4S(C)(=O)=O)cc3)C2)C1] | 496.2 [M + H]⁺ |
| 567 | [3-[[2-Fluoro-4- (trifluoromethyl)phenyl]methyl- methyl-amino]azetidin-1-yl]- [(3S)-3-(1,2,4-triazol-4- yl)pyrrolidin-1-yl]methanone | 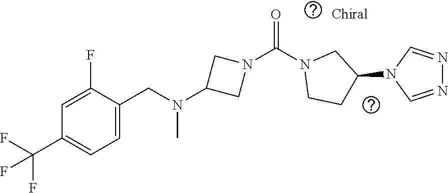 [SMILES: CN(Cc1ccc(C(F)(F)F)cc1F)C1CN(C(=O)N2CC[C@H](n3cnnc3)C2)C1] | 427.2 [M + H]⁺ |
| 568 | N-[1-[3-[4-[1- (Trifluoromethyl)cyclopropyl] phenyl]azetidine-1- carbonyl]azetidin-3- yl]methanesulfonamide | 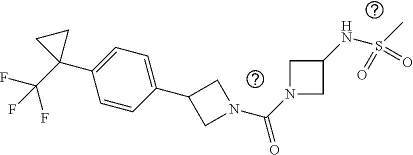 [SMILES: CS(=O)(=O)NC1CN(C(=O)N2CC(c3ccc(C4(C(F)(F)F)CC4)cc3)C2)C1] | 418.2 [M + H]⁺ |
| 569 | N-[1-[3-[4-[[1- (Trifluoromethyl)cyclopropyl] methoxy]phenyl]azetidine-1- carbonyl]azetidin-3- yl]methanesulfonamide | 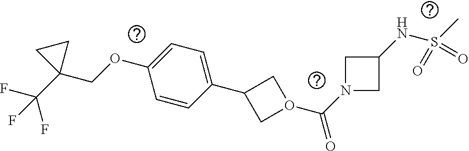 | 448.2 [M + H]⁺ |
| 570 | N-[1-[3-[4-[5- (Trifluoromethyl)pyrazin-2- yl]oxyphenyl]azetidine-1- carbonyl]azetidin-3- yl]methanesulfonamide | 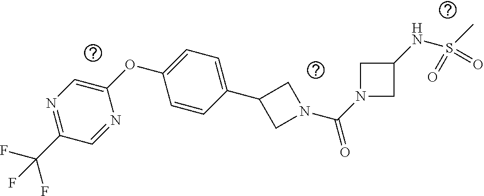 [SMILES: CS(=O)(=O)NC1CN(C(=O)N2CC(c3ccc(Oc4cnc(C(F)(F)F)cn4)cc3)C2)C1] | 472.2 [M + H]⁺ |
| 571 | N-[1-[3-[6-[3- (Trifluoromethyl)azetidin-1-yl]- 3-pyridyl]azetidine-1- carbonyl]azetidin-3- yl]methanesulfonamide | 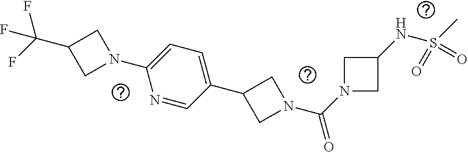 [SMILES: CS(=O)(=O)NC1CN(C(=O)N2CC(c3ccc(N4CC(C(F)(F)F)C4)nc3)C2)C1] | 434.2 [M + H]⁺ |
| 572 | (3S)-1-[3-[3-[5-(2,2- Dimethylpropyl)-1,3,4- oxadiazol-2-yl]-1- bicyclo[1.1.1]pentanyl]azetidine- 1-carbonyl]pyrrolidine-3- carboxamide | 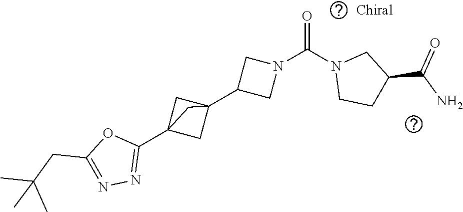 [SMILES: CC(C)(C)Cc1nnc(C23CC(C4CN(C(=O)N5CC[C@H](C(N)=O)C5)C4)(C2)C3)o1] | 402.3 [M + H]⁺ |
| 573 | [3-[3-[5-(2,2-Dimethylpropyl)- 1,3,4-oxadiazol-2-yl]-1- bicyclo[1.1.1]pentanyl]azetidin- 1-yl]-[(3S)-3-(1H-1,2,4-triazol- 5-yl)pyrrolidin-1-yl]methanone | 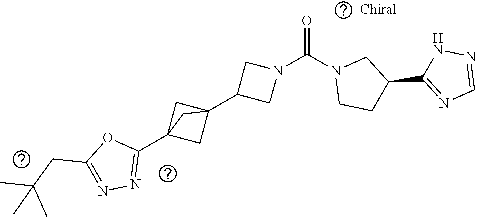 [SMILES: CC(C)(C)Cc1nnc(C23CC(C4CN(C(=O)N5CC[C@H](c6ncn[nH]6)C5)C4)(C2)C3)o1] | 426.3 [M + H]⁺ |
| 574 | (3S)-1-[3-[4-[3-(2,2- Dimethylpropyl)triazol-4- yl]phenyl]azetidine-1- carbonyl]pyrrolidine-3- carboxamide | 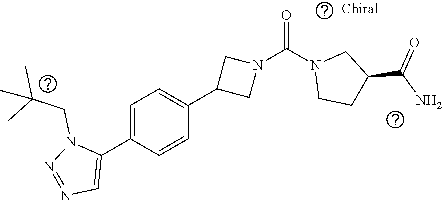 [SMILES: CC(C)(C)Cn1nncc1-c1ccc(C2CN(C(=O)N3CC[C@H](C(N)=O)C3)C2)cc1] | 411.3 [M + H]⁺ |
| 575 | rac-(3S)-1-[3-[6-(2,2-Difluoro-5- azaspiro[2.4]heptan-5-yl)-3- pyridyl]azetidine-1- carbonyl]pyrrolidine-3- carboxamide | 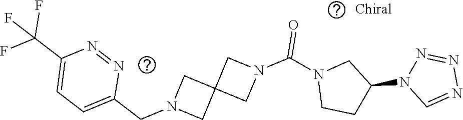 [SMILES: O=C(N1CC[C@H](n2cnnn2)C1)N1CC2(CN(Cc3ccc(C(F)(F)F)nn3)C2)C1] | 406.2 [M + H]⁺ |
| 576 | [(3S)-3-(Tetrazol-1- yl)pyrrolidin-1-yl]-[6-[[6- (trifluoromethyl)pyridazin-3- yl]methyl]-2,6- diazaspiro[3.3]heptan-2- yl]methanone | 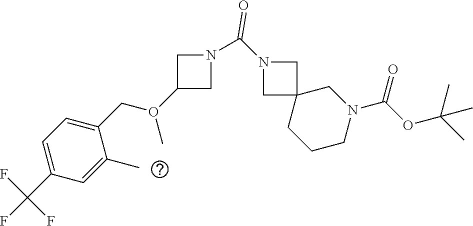 | 424.2 [M + H]⁺ |
| 577 | tert-Butyl 2-[3-[[2-fluoro-4- (trifluoromethyl)phenyl]methyl- methyl-amino]azetidine-1- carbonyl]-2,8- diazaspiro[3.5]nonane-8- carboxylate | 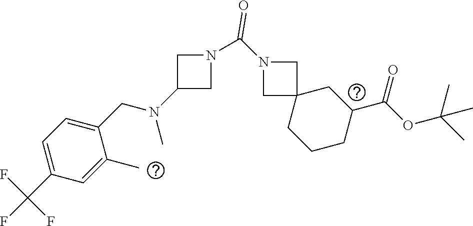 | 515.2 [M + H]⁺ |
| 578 | N-[1-[2-[[2-Fluoro-4- (trifluoromethyl)phenyl]methyl]- 2,7-diazaspiro[3.5]nonane-7- carbonyl]azetidin-3- yl]methanesulfonamide | 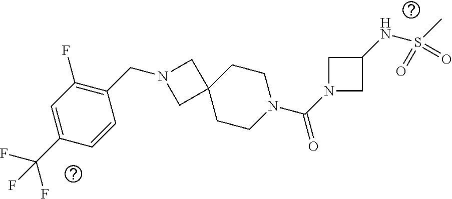 [SMILES: CS(=O)(=O)NC1CN(C(=O)N2CCC3(CC2)CN(Cc2ccc(C(F)(F)F)cc2F)C3)C1] | 479.2 [M + H]⁺ |
| 579 | (3S)-1-[3-[6-(6-Oxa-2- azaspiro[3.4]octan-2-yl)-3- pyridyl]azetidine-1- carbonyl]pyrrolidine-3- carboxamide | 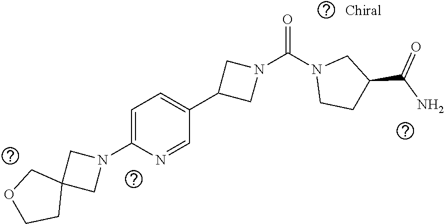 [SMILES: NC(=O)[C@H]1CCN(C(=O)N2CC(c3ccc(N4CC5(CCOC5)C4)nc3)C2)C1] | 386.2 [M + H]⁺ |
| 580 | (3S)-1-[3-[6-(5-Oxa-2- azaspiro[3.4]octan-2-yl)-3- pyridyl]azetidine-1- carbonyl]pyrrolidine-3- carboxamide | 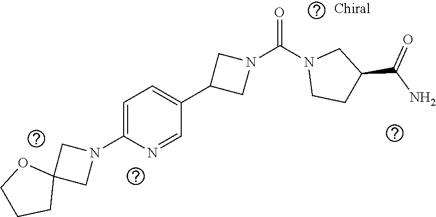 [SMILES: NC(=O)[C@H]1CCN(C(=O)N2CC(c3ccc(N4CC5(CCCO5)C4)nc3)C2)C1] | 386.2 [M + H]⁺ |
| 581 | [7-[[6-(Difluoromethoxy)-3- pyridyl]methyl]-2- azaspiro[3.5]nonan-2-yl]-[(3S)- 3-(tetrazol-1-yl)pyrrolidin-1- yl]methanone | 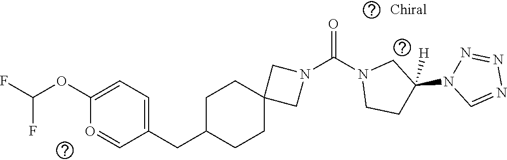 | 448.2 [M + H]⁺ |
| 582 | [6-[(2,4- Difluorophenyl)methyl]-2- azaspiro[3.3]heptan-2-yl]-[(3S)- 3-(tetrazol-1-yl)pyrrolidin-1- yl]methanone | 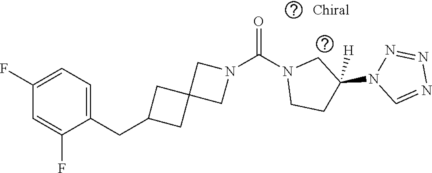 [SMILES: O=C(N1CC[C@H](n2cnnn2)C1)N1CC2(CC(Cc3ccc(F)cc3F)C2)C1] | 389.2 [M + H]⁺ |
| 584 | [6-[[4-Fluoro-2- (trifluoromethyl)phenyl]methyl]- 2-azaspiro[3.3]heptan-2-yl]- [(3S)-3-(tetrazol-1-yl)pyrrolidin- 1-yl]methanone | 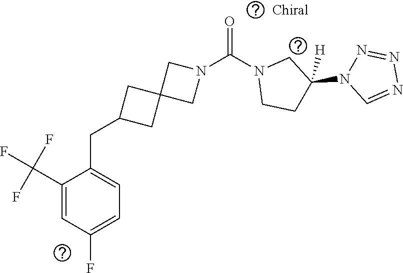 [SMILES: O=C(N1CC[C@H](n2cnnn2)C1)N1CC2(CC(Cc3ccc(F)cc3C(F)(F)F)C2)C1] | 439.2 [M + H]⁺ |
| 585 | N-[1-[3-[4-[1- (Trifluoromethyl)cyclopropyl] phenyl]azetidine-1- carbonyl]azetidin-3-yl]acetamide | 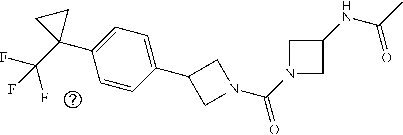 [SMILES: CC(=O)NC1CN(C(=O)N2CC(c3ccc(C4(C(F)(F)F)CC4)cc3)C2)C1] | 382.2 [M + H]⁺ |
| 586 | N-[1-[3-[[2-Fluoro-4- (trifluoromethyl)phenyl]methoxy] azetidine-1-carbonyl]azetidin-3- yl]acetamide | 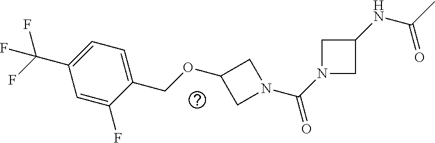 [SMILES: CC(=O)NC1CN(C(=O)N2CC(OCc3ccc(C(F)(F)F)cc3F)C2)C1] | 390.2 [M + H]⁺ |
| 587 | N-[1-[7-[[1- (Trifluoromethyl)cyclopropyl] methoxy]-2-azaspiro[3.5]nonane- 2-carbonyl]azetidin-3- yl]acetamide | 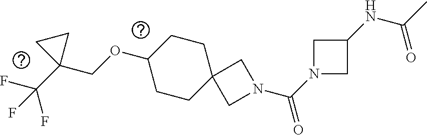 [SMILES: CC(=O)NC1CN(C(=O)N2CC3(CCC(OCC4(C(F)(F)F)CC4)CC3)C2)C1] | 404.2 [M + H]⁺ |
| 588 | N-[1-[3-[4-[5- (Trifluoromethyl)pyrazin-2- yl]oxyphenyl]azetidine-1- carbonyl]azetidin-3-yl]acetamide | 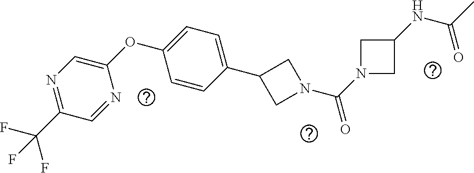 [SMILES: CC(=O)NC1CN(C(=O)N2CC(c3ccc(Oc4cnc(C(F)(F)F)cn4)cc3)C2)C1] | 436.2 [M + H]⁺ |
| 589 | (3S)-1-[3-[6-[(3R)-3- (Trifluoromethyl)pyrrolidin-1- yl]-3-pyridyl]azetidine-1- carbonyl]pyrrolidine-3- carboxamide | 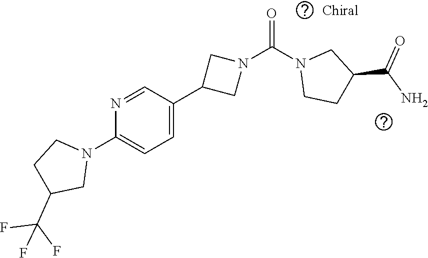 [SMILES: NC(=O)[C@H]1CCN(C(=O)N2CC(c3ccc(N4CCC(C(F)(F)F)C4)nc3)C2)C1] | 412.2 [M + H]⁺ |
| 590 | (3S)-1-[3-[6-[(3S)-3- (Trifluoromethyl)pyrrolidin-1- yl]-3-pyridyl]azetidine-1- carbonyl]pyrrolidine-3- carboxamide | 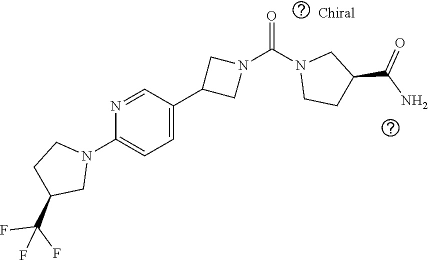 [SMILES: NC(=O)[C@H]1CCN(C(=O)N2CC(c3ccc(N4CC[C@H](C(F)(F)F)C4)nc3)C2)C1] | 412.2 [M + H]⁺ |
| 591 | (3S)-1-[3-[4-[5- (Trifluoromethyl)pyrazin-2- yl]oxyphenyl]azetidine-1- carbonyl]pyrrolidine-3- carboxamide | 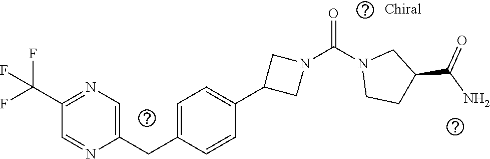 [SMILES: NC(=O)[C@H]1CCN(C(=O)N2CC(c3ccc(Cc4cnc(C(F)(F)F)cn4)cc3)C2)C1] | 436.2 [M + H]⁺ |
| 592 | (3S)-1-[3-[5-[[1- (Trifluoromethyl)cyclopropyl] methylamino]-2- pyridyl]azetidine- 1-carbonyl]pyrrolidine-3- carboxamide | 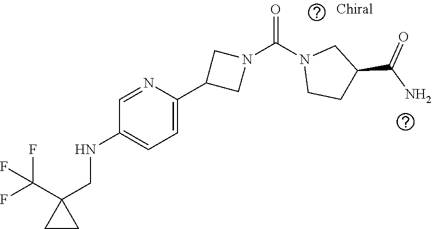 [SMILES: NC(=O)[C@H]1CCN(C(=O)N2CC(c3ccc(NCC4(C(F)(F)F)CC4)cn3)C2)C1] | 412.2 [M + H]⁺ |
| 593 | (3S)-1-[3-[5-[[1- (Trifluoromethyl)cyclopropyl] methylamino]pyrazin-2- yl]azetidine-1- carbonyl]pyrrolidine-3- carboxamide | 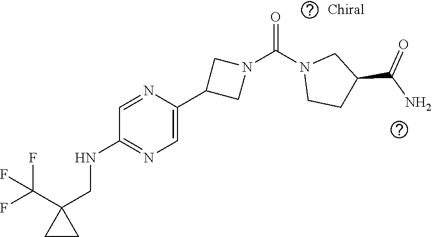 [SMILES: NC(=O)[C@H]1CCN(C(=O)N2CC(c3cnc(NCC4(C(F)(F)F)CC4)cn3)C2)C1] | 413.2 [M + H]⁺ |
| 594 | (3S)-1-[3-[6-[3- (Trifluoromethyl)azetidin-1-yl]- 3-pyridyl]azetidine-1- carbonyl]pyrrolidine-3- carboxamide | 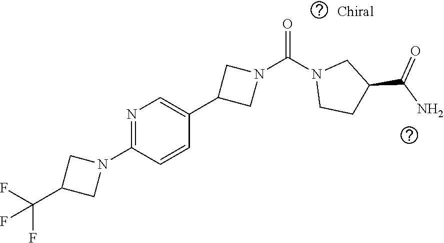 [SMILES: NC(=O)[C@H]1CCN(C(=O)N2CC(c3ccc(N4CC(C(F)(F)F)C4)nc3)C2)C1] | 398.2 [M + H]⁺ |
| 595 | (3S)-1-[3-[2-[3- (Trifluoromethyl)azetidin-1- yl]pyrimidin-5-yl]azetidine-1- carbonyl]pyrrolidine-3- carboxamide | 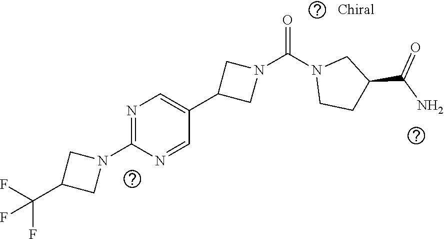 [SMILES: NC(=O)[C@H]1CCN(C(=O)N2CC(c3cnc(N4CC(C(F)(F)F)C4)nc3)C2)C1] | 399.2 [M + H]⁺ |
| 596 | (3S)-1-[7-[(3,5-Difluoro-2- pyridyl)methyl]-2- azaspiro[3.5]nonane-2- carbonyl]pyrrolidine-3- carboxamide | 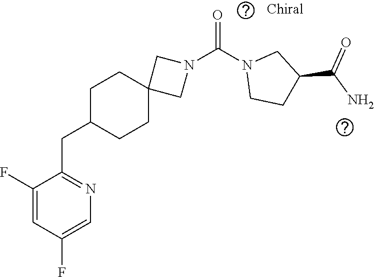 [SMILES: NC(=O)[C@H]1CCN(C(=O)N2CC3(CCC(Cc4ncc(F)cc4F)CC3)C2)C1] | 393.2 [M + H]⁺ |
| 597 | (3S)-1-[7-[(5-Chloro-2- pyridyl)methyl]-2- azaspiro[3.5]nonane-2- carbonyl]pyrrolidine-3- carboxamide | 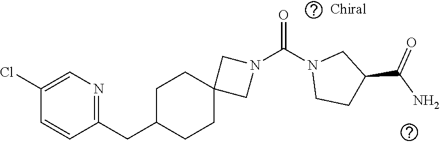 [SMILES: NC(=O)[C@H]1CCN(C(=O)N2CC3(CCC(Cc4ccc(Cl)cn4)CC3)C2)C1] | 391.2 [M + H]⁺ |
| 598 | (3S)-1-[7-[[5- (Trifluoromethyl)pyrazin-2- yl]methyl]-2- azaspiro[3.5]nonane-2- carbonyl]pyrrolidine-3- carboxamide | 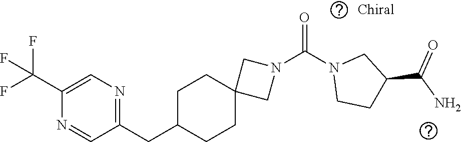 [SMILES: NC(=O)[C@H]1CCN(C(=O)N2CC3(CCC(Cc4cnc(C(F)(F)F)cn4)CC3)C2)C1] | 426.2 [M + H]⁺ |
| 599 | (3S)-1-[7-[[5-(Trifluoromethyl)- 2-pyridyl]methyl]-2- azaspiro[3.5]nonane-2- carbonyl]pyrrolidine-3- carboxamide | 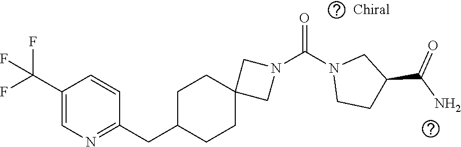 [SMILES: NC(=O)[C@H]1CCN(C(=O)N2CC3(CCC(Cc4ccc(C(F)(F)F)cn4)CC3)C2)C1] | 425.2 [M + H]⁺ |
| 600 | (3S)-1-[7-[[6- (Difluoromethoxy)-3- pyridyl]methyl]-2- azaspiro[3.5]nonane-2- carbonyl]pyrrolidine-3- carboxamide | 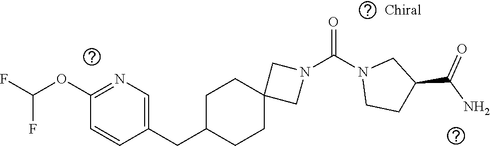 [SMILES: NC(=O)[C@H]1CCN(C(=O)N2CC3(CCC(Cc4ccc(OC(F)F)nc4)CC3)C2)C1] | 423.2 [M + H]⁺ |
| 601 | (3S)-1-[6-[[6- (Trifluoromethyl)pyridazin-3- yl]methyl]-2- azaspiro[3.3]heptane-2- carbonyl]pyrrolidine-3- carboxamide | 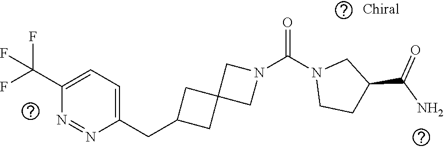 [SMILES: NC(=O)[C@H]1CCN(C(=O)N2CC3(CC(Cc4ccc(C(F)(F)F)nn4)C3)C2)C1] | 398.2 [M + H]⁺ |
| 602 | (3S)-1-[6-[[2- (Trifluoromethyl)pyrimidin-5- yl]methyl]-2- azaspiro[3.3]heptane-2- carbonyl]pyrrolidine-3- carboxamide | 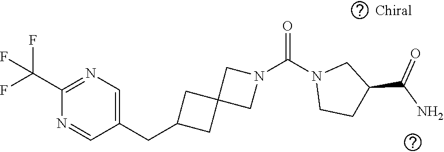 [SMILES: NC(=O)[C@H]1CCN(C(=O)N2CC3(CC(Cc4cnc(C(F)(F)F)nc4)C3)C2)C1] | 398.2 [M + H]⁺ |
| 603 | (3S)-1-[(6S)-6-[5- (Trifluoromethyl)pyrazin-2- yl]oxy-2-azaspiro[3.4]octane-2- carbonyl]pyrrolidine-3- carboxamide | 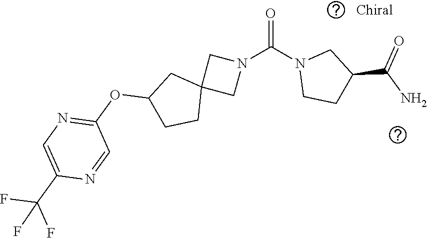 [SMILES: NC(=O)[C@H]1CCN(C(=O)N2CC3(CCC(Oc4cnc(C(F)(F)F)cn4)C3)C2)C1] | 414.2 [M + H]⁺ |
| 604 | [6-(4-Fluorophenyl)-2- azaspiro[3.3]heptan-2-yl]-[(3S)- 3-(tetrazol-1-yl)pyrrolidin-1- yl]methanone | 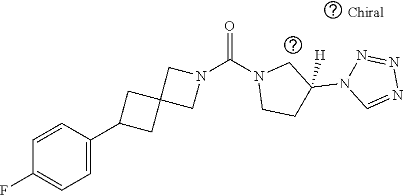 [SMILES: O=C(N1CC[C@H](n2cnnn2)C1)N1CC2(CC(c3ccc(F)cc3)C2)C1] | 357.2 [M + H]⁺ |
| 605 | [(3S)-3-(Tetrazol-1- yl)pyrrolidin-1-yl]-[6-[[4- (trifluoromethylsulfonyl)phenyl] methyl]-2,6- diazaspiro[3.3]heptan-2- yl]methanone | 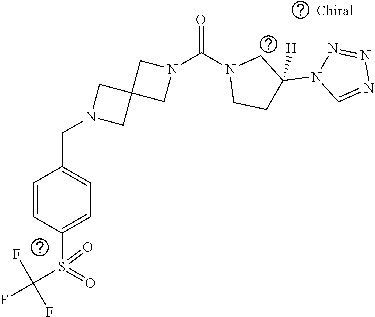 [SMILES: O=C(N1CC[C@H](n2cnnn2)C1)N1CC2(CN(Cc3ccc(S(=O)(=O)C(F)(F)F)cc3)C2)C1] | 486.2 [M + H]⁺ |
| 606 | (3S)-1-[3-[4-(5-Cyclobutyl-4H- 1,2,4-triazol-3- yl)phenyl]azetidine-1- carbonyl]pyrrolidine-3- carboxamide | 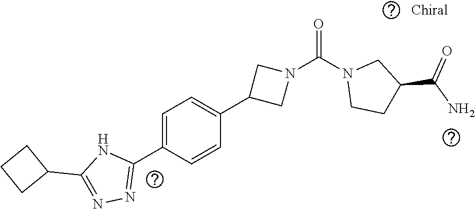 [SMILES: NC(=O)[C@H]1CCN(C(=O)N2CC(c3ccc(-c4nnc(C5CCC5)[nH]4)cc3)C2)C1] | 395.3 [M + H]⁺ |
| 607 | (3S)-1-[6-(3-Cyclopropyl-1,2,4- triazol-1-yl)-2- azaspiro[3.3]heptane-2- carbonyl]pyrrolidine-3- carboxamide | 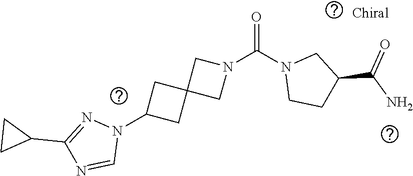 [SMILES: NC(=O)[C@H]1CCN(C(=O)N2CC3(CC(n4cnc(C5CC5)n4)C3)C2)C1] | 345.2 [M + H]⁺ |
| 608 | [6-[(2,4- Difluorophenyl)methyl]-2- azaspiro[3.3]heptan-2-yl]-[(3S)- 3-(triazol-1-yl)pyrrolidin-1- yl]methanone | 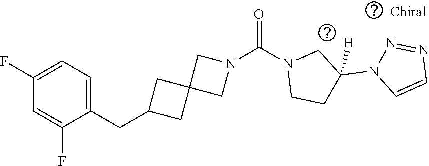 [SMILES: O=C(N1CC[C@H](n2ccnn2)C1)N1CC2(CC(Cc3ccc(F)cc3F)C2)C1] | 388.2 [M + H]⁺ |
| 609 | [3-[6-(2-Chlorophenoxy)-3- pyridyl]azetidin-1-yl]-[(3R)-3- (triazol-1-yl)pyrrolidin-1- yl]methanone | 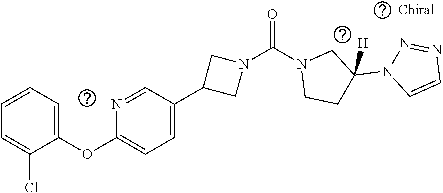 [SMILES: O=C(N1CC(c2ccc(Oc3ccccc3Cl)nc2)C1)N1CC[C@@H](n2ccnn2)C1] | 425.2 [M + H]⁺ |
| 611 | [6-[(2,4- Difluorophenyl)methyl]-2- azaspiro[3.3]heptan-2-yl]-[(3R)- 3-(triazol-1-yl)pyrrolidin-1- yl]methanone | 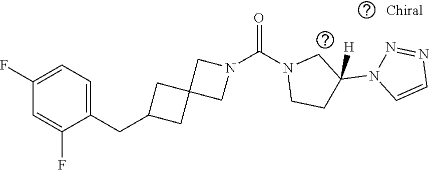 [SMILES: O=C(N1CC[C@@H](n2ccnn2)C1)N1CC2(CC(Cc3ccc(F)cc3F)C2)C1] | 388.2 [M + H]⁺ |
| 612 | [3-[4-(4-Chloro-2- methylsulfonyl- phenyl)phenyl]azetidin-1-yl]- [(3R)-3-(triazol-2-yl)pyrrolidin- 1-yl]methanone | 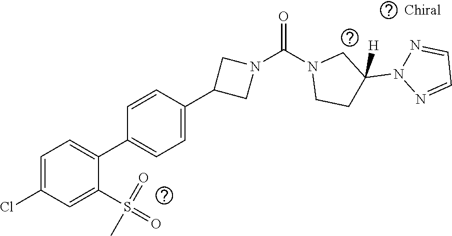 [SMILES: CS(=O)(=O)c1cc(Cl)ccc1-c1ccc(C2CN(C(=O)N3CC[C@@H](n4nccn4)C3)C2)cc1] | 486.2 [M + H]⁺ |
| 613 | [6-[(2,4- Difluorophenyl)methyl]-2- azaspiro[3.3]heptan-2-yl]-[(3R)- 3-(triazol-2-yl)pyrrolidin-1- yl]methanone | 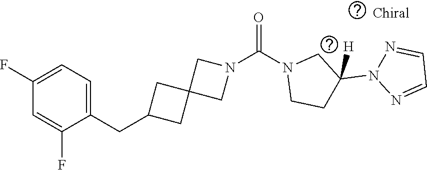 [SMILES: O=C(N1CC[C@@H](n2nccn2)C1)N1CC2(CC(Cc3ccc(F)cc3F)C2)C1] | 388.2 [M + H]⁺ |
| 614 | [3-[[2-Fluoro-4- (trifluoromethyl)phenyl]methyl- methyl-amino]azetidin-1-yl]- [(3R)-3-(triazol-2-yl)pyrrolidin- 1-yl]methanone | 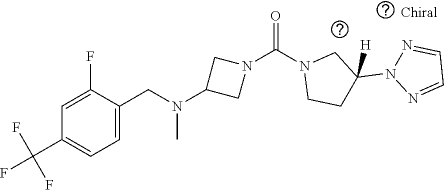 [SMILES: CN(Cc1ccc(C(F)(F)F)cc1F)C1CN(C(=O)N2CC[C@@H](n3nccn3)C2)C1] | 427.2 [M + H]⁺ |
| 615 | [3-[4-(4-Chloro-2- methylsulfonyl- phenyl)phenyl]azetidin-1-yl]- [(3S)-3-(triazol-1-yl)pyrrolidin- 1-yl]methanone | 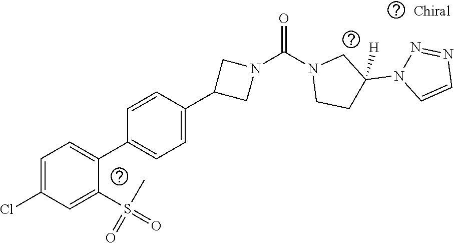 [SMILES: CS(=O)(=O)c1cc(Cl)ccc1-c1ccc(C2CN(C(=O)N3CC[C@H](n4ccnn4)C3)C2)cc1] | 486.2 [M + H]⁺ |
| 616 | [7-[[6-(Difluoromethoxy)-3- pyridyl]methyl]-2- azaspiro[3.5]nonan-2-yl]-[(3R)- 3-(triazol-1-yl)pyrrolidin-1- yl]methanone | 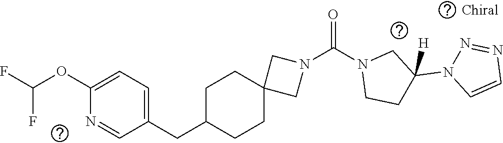 [SMILES: O=C(N1CC[C@@H](n2ccnn2)C1)N1CC2(CCC(Cc3ccc(OC(F)F)nc3)CC2)C1] | 447.3 [M + H]⁺ |
| 617 | [3-[4-(4-Chloro-2- methylsulfonyl- phenyl)phenyl]azetidin-1-yl]- [(3S)-3-(triazol-2-yl)pyrrolidin- 1-yl]methanone | 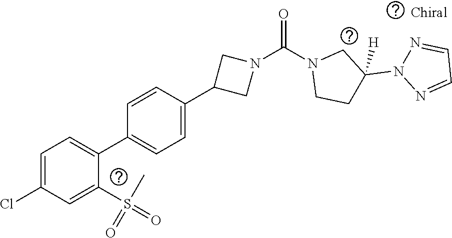 [SMILES: CS(=O)(=O)c1cc(Cl)ccc1-c1ccc(C2CN(C(=O)N3CC[C@H](n4nccn4)C3)C2)cc1] | 486.2 [M + H]⁺ |
| 618 | [3-[4-(4-Chloro-2- methylsulfonyl- phenyl)phenyl]azetidin-1-yl]-[6- (triazol-2-yl)-2- azaspiro[3.3]heptan-2- yl]methanone | 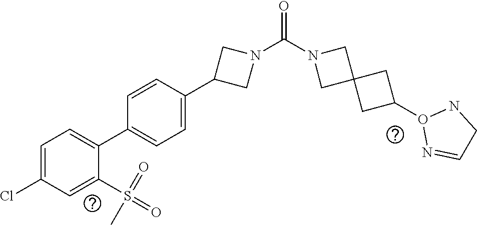 | 512.2 [M + H]⁺ |
| 619 | [3-[4-(4-Chloro-2- methylsulfonyl- phenyl)phenyl]azetidin-1-yl]-[6- (triazol-1-yl)-2- azaspiro[3.3]heptan-2- yl]methanone | 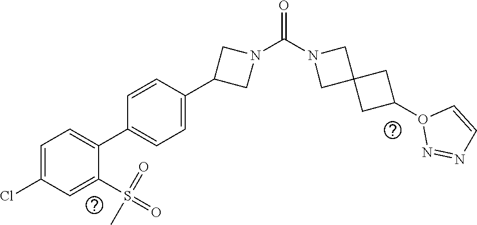 | 512.2 [M + H]⁺ |
| 620 | (3S)-1-[3-[4-(3-Cyclopropyl-5- methyl-pyrazol-1- yl)phenyl]azetidine-1- carbonyl]pyrrolidine-3- carboxamide | 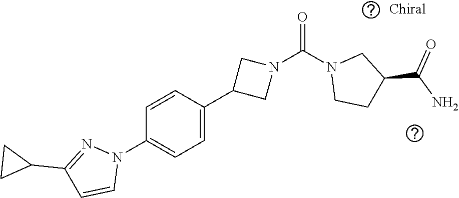 [SMILES: NC(=O)[C@H]1CCN(C(=O)N2CC(c3ccc(-n4ccc(C5CC5)n4)cc3)C2)C1] | 394.2 [M + H]⁺ |
| 622 | (3S)-1-[7-(4-Chloro-2- methylsulfonyl-phenyl)-2- azaspiro[3.5]nonane-2- carbonyl]pyrrolidine-3- carboxamide | 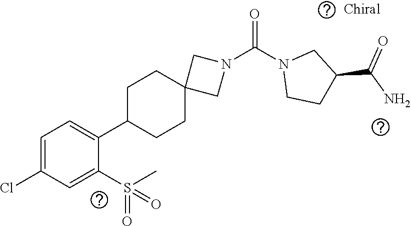 [SMILES: CS(=O)(=O)c1cc(Cl)ccc1C1CCC2(CC1)CN(C(=O)N1CC[C@H](C(N)=O)C1)C2] | 454.3 [M + H]⁺ |
| 624 | (3S)-1-[3-[4-(5-Cyclopropyl-4H- 1,2,4-triazol-3- yl)phenyl]azetidine-1- carbonyl]pyrrolidine-3- carboxamide | 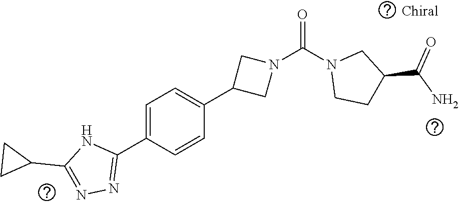 [SMILES: NC(=O)[C@H]1CCN(C(=O)N2CC(c3ccc(-c4nnc(C5CC5)[nH]4)cc3)C2)C1] | 381.3 [M + H]⁺ |
| 625 | (3S)-1-[3-[4-[5-[1- (Trifluoromethyl)cyclopropyl]- 4H-1,2,4-triazol-3- yl]phenyl]azetidine-1- carbonyl]pyrrolidine-3- carboxamide | 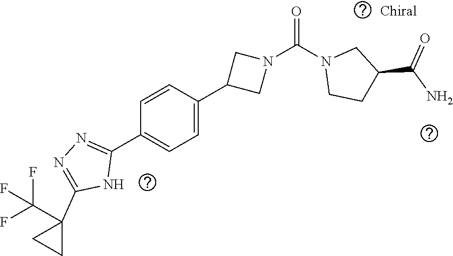 [SMILES: NC(=O)[C@H]1CCN(C(=O)N2CC(c3ccc(-c4nnc(C5(C(F)(F)F)CC5)[nH]4)cc3)C2)C1] | 449.3 [M + H]⁺ |
| 626 | (3S)-1-[6-[[5- (Trifluoromethyl)pyrimidin-2- yl]methyl]-2- azaspiro[3.3]heptane-2- carbonyl]pyrrolidine-3- carboxamide | 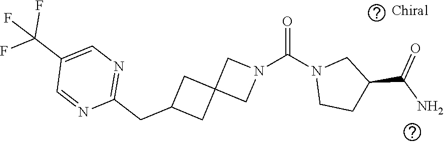 [SMILES: NC(=O)[C@H]1CCN(C(=O)N2CC3(CC(Cc4ncc(C(F)(F)F)cn4)C3)C2)C1] | 398.2 [M + H]⁺ |
| 627 | [(3S)-3-(1H-1,2,4-Triazol-5- yl)pyrrolidin-1-yl]-[6-[[5- (trifluoromethyl)pyrimidin-2- yl]methyl]-2- azaspiro[3.3]heptan-2- yl]methanone | 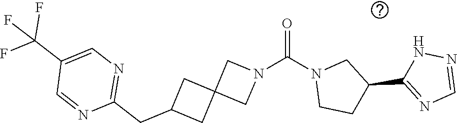 [SMILES: O=C(N1CC[C@H](c2ncn[nH]2)C1)N1CC2(CC(Cc3ncc(C(F)(F)F)cn3)C2)C1] | 422.2 [M + H]⁺ |
| 630 | rac-(3S)-1-[6-[[6- (Trifluoromethyl)-3- pyridyl]methyl]-2- azaspiro[3.4]octane-2- carbonyl]pyrrolidine-3- carboxamide | 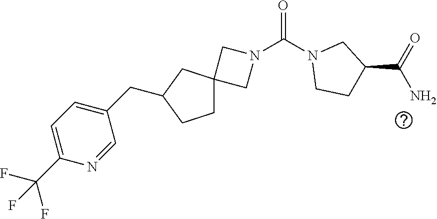 [SMILES: NC(=O)[C@H]1CCN(C(=O)N2CC3(CCC(Cc4ccc(C(F)(F)F)nc4)C3)C2)C1] | 411.3 [M + H]⁺ |
| 631 | [rac-(3S)-3-(1H-1,2,4-Triazol-5- yl)pyrrolidin-1-yl]-[6-[[6- (trifluoromethyl)-3- pyridyl]methyl]-2- azaspiro[3.4]octan-2- yl]methanone | 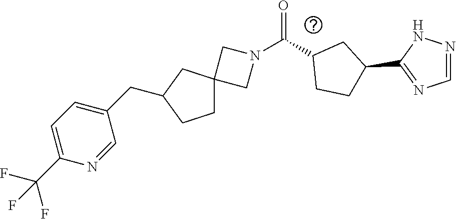 | 435.3 [M + H]⁺ |
| 632 | (3S)-1-[7-(4-Chloro-2-fluoro- phenyl)-2,7- diazaspiro[3.5]nonane-2- carbonyl]pyrrolidine-3- carboxamide | 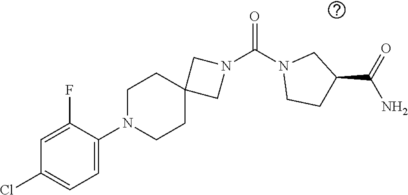 [SMILES: NC(=O)[C@H]1CCN(C(=O)N2CC3(CCN(c4ccc(Cl)cc4F)CC3)C2)C1] | 395.2 [M + H]⁺ |
| 633 | (3S)-1-[7-(5-Chloro-3-fluoro-2- pyridyl)-2,7- diazaspiro[3.5]nonane-2- carbonyl]pyrrolidine-3- carboxamide | 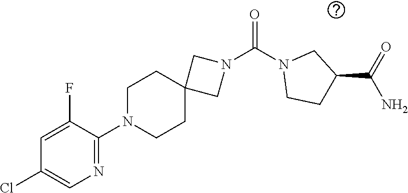 [SMILES: NC(=O)[C@H]1CCN(C(=O)N2CC3(CCN(c4ncc(Cl)cc4F)CC3)C2)C1] | 396.2 [M + H]⁺ |
| 634 | 1-[4-[1-[(3S)-3-(1H-1,2,4- Triazol-5-yl)pyrrolidine-1- carbonyl]azetidin-3-yl]phenyl]- 4-(trifluoromethyl)pyridin-2-one | 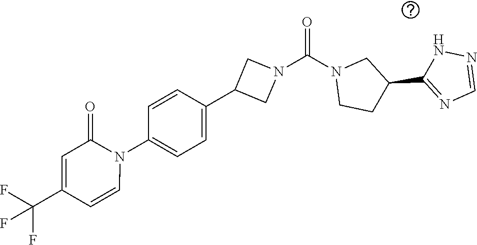 [SMILES: O=C(N1CC(c2ccc(-n3ccc(C(F)(F)F)cc3=O)cc2)C1)N1CC[C@H](c2ncn[nH]2)C1] | 459.3 [M + H]⁺ |
| 635 | [3-[4-(3-Cyclopropyl-5-methyl- pyrazol-1-yl)phenyl]azetidin-1- yl]-[(3S)-3-(1H-1,2,4-triazol-5- yl)pyrrolidin-1-yl]methanone | 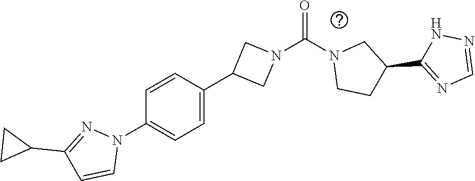 [SMILES: O=C(N1CC(c2ccc(-n3ccc(C4CC4)n3)cc2)C1)N1CC[C@H](c2ncn[nH]2)C1] | 416.4 [M − H]⁻ |
| 636 | [(3S)-3-(1H-1,2,4-Triazol-5- yl)pyrrolidin-1-yl]-[3-[6-[(3R)- 3-(trifluoromethyl)pyrrolidin-1- yl]-3-pyridyl]azetidin-1- yl]methanone | 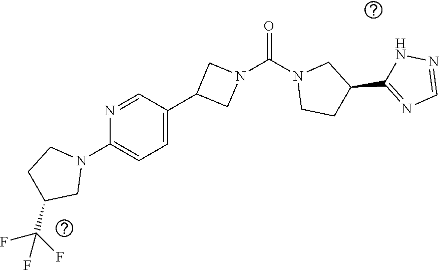 [SMILES: O=C(N1CC(c2ccc(N3CC[C@@H](C(F)(F)F)C3)nc2)C1)N1CC[C@H](c2ncn[nH]2)C1] | 436.4 [M + H]⁺ |
| 637 | [(3S)-3-(1H-1,2,4-Triazol-5- yl)pyrrolidin-1-yl]-[3-[6-[(3S)-3- (trifluoromethyl)pyrrolidin-1- yl]-3-pyridyl]azetidin-1- yl]methanone | 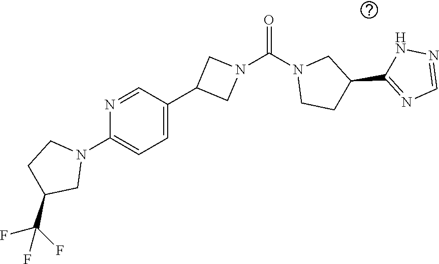 [SMILES: O=C(N1CC(c2ccc(N3CC[C@H](C(F)(F)F)C3)nc2)C1)N1CC[C@H](c2ncn[nH]2)C1] | 436.3 [M + H]⁺ |
| 638 | [(3S)-3-(1H-1,2,4-Triazol-5- yl)pyrrolidin-1-yl]-[3-[6-[3- (trifluoromethyl)azetidin-1-yl]- 3-pyridyl]azetidin-1- yl]methanone | 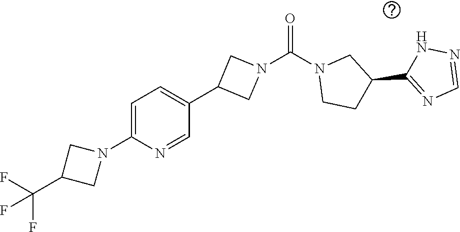 [SMILES: O=C(N1CC(c2ccc(N3CC(C(F)(F)F)C3)nc2)C1)N1CC[C@H](c2ncn[nH]2)C1] | 422.3 [M + H]⁺ |
| 639 | [(3S)-3-(1H-1,2,4-Triazol-5- yl)pyrrolidin-1-yl]-[3-[2-[3- (trifluoromethyl)azetidin-1- yl]pyrimidin-5-yl]azetidin-1- yl]methanone | 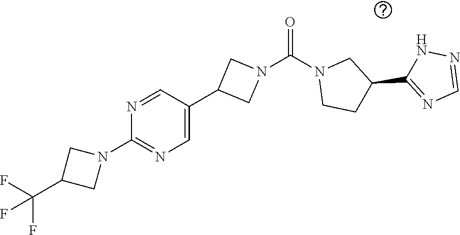 [SMILES: O=C(N1CC(c2cnc(N3CC(C(F)(F)F)C3)nc2)C1)N1CC[C@H](c2ncn[nH]2)C1] | 423.3 [M + H]⁺ |
| 640 | [3-[4-(2-Chloro-4- methylsulfonyl- phenyl)phenyl]azetidin-1-yl]- [(3S)-3-(1H-1,2,4-triazol-5- yl)pyrrolidin-1-yl]methanone | 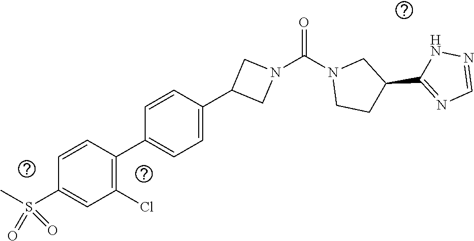 [SMILES: CS(=O)(=O)c1ccc(-c2ccc(C3CN(C(=O)N4CC[C@H](c5ncn[nH]5)C4)C3)cc2)c(Cl)c1] | 486.3 [M + H]⁺ |
| 641 | [3-[5-(4-Chloro-2- methylsulfonyl-phenyl)-2- pyridyl]azetidin-1-yl]-[(3S)-3- (1H-1,2,4-triazol-5- yl)pyrrolidin-1-yl]methanone | 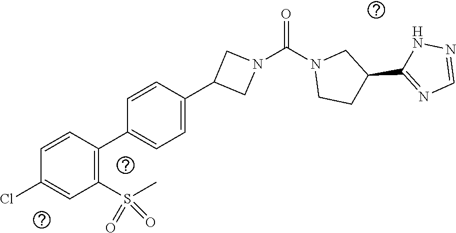 [SMILES: CS(=O)(=O)c1cc(Cl)ccc1-c1ccc(C2CN(C(=O)N3CC[C@H](c4ncn[nH]4)C3)C2)cc1] | 487.3 [M + H]⁺ |
| 642 | [7-[(5-Fluoro-2-pyridyl)methyl]- 2-azaspiro[3.5]nonan-2-yl]- [(3S)-3-(1H-1,2,4-triazol-5- yl)pyrrolidin-1-yl]methanone | 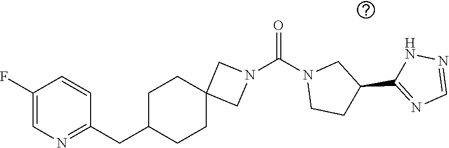 [SMILES: O=C(N1CC[C@H](c2ncn[nH]2)C1)N1CC2(CCC(Cc3ccc(F)cn3)CC2)C1] | 399.4 [M + H]⁺ |
| 643 | [(3S)-3-(1H-1,2,4-Triazol-5- yl)pyrrolidin-1-yl]-[3-[5-[[1- (trifluoromethyl)cyclopropyl] methylamino]pyrazin-2-yl] azetidin-1-yl]methanone | 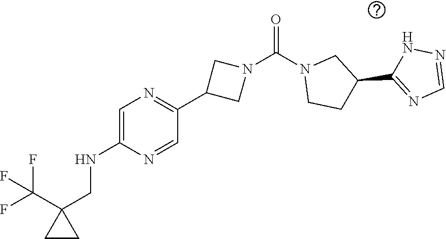 [SMILES: O=C(N1CC(c2cnc(NCC3(C(F)(F)F)CC3)cn2)C1)N1CC[C@H](c2ncn[nH]2)C1] | 437.4 [M + H]⁺ |
| 644 | [7-[(5-Chloro-2-pyridyl)methyl]- 2-azaspiro[3.5]nonan-2-yl]- [(3S)-3-(1H-1,2,4-triazol-5- yl)pyrrolidin-1-yl]methanone | 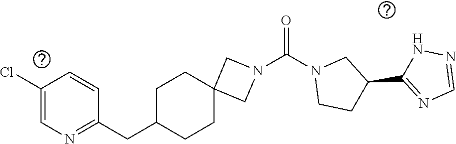 [SMILES: O=C(N1CC[C@H](c2ncn[nH]2)C1)N1CC2(CCC(Cc3ccc(Cl)cn3)CC2)C1] | 415.3 [M + H]⁺ |
| 645 | [(3S)-3-(1H-1,2,4-Triazol-5- yl)pyrrolidin-1-yl]-[7-[[5- (trifluoromethyl)pyrazin-2- yl]methyl]-2- azaspiro[3.5]nonan-2- yl]methanone | 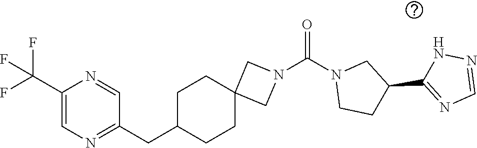 [SMILES: O=C(N1CC[C@H](c2ncn[nH]2)C1)N1CC2(CCC(Cc3cnc(C(F)(F)F)cn3)CC2)C1] | 450.4 [M + H]⁺ |
| 646 | [7-[[6-(Difluoromethoxy)-3- pyridyl]methyl]-2- azaspiro[3.5]nonan-2-yl]-[(3S)- 3-(1H-1,2,4-triazol-5- yl)pyrrolidin-1-yl]methanone | 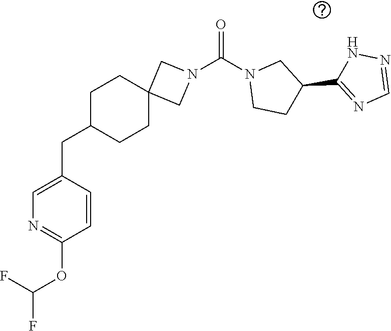 [SMILES: O=C(N1CC[C@H](c2ncn[nH]2)C1)N1CC2(CCC(Cc3ccc(OC(F)F)nc3)CC2)C1] | 447.4 [M + H]⁺ |
| 647 | [(3S)-3-(1H-1,2,4-Triazol-5- yl)pyrrolidin-1-yl]-[7-[[1- (trifluoromethyl)cyclopropyl] methoxy]-2-azaspiro[3.5]nonan- 2-yl]methanone | 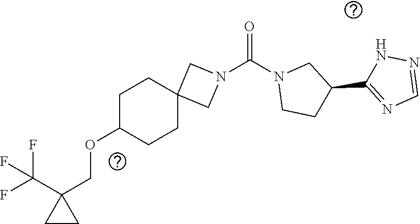 [SMILES: O=C(N1CC[C@H](c2ncn[nH]2)C1)N1CC2(CCC(OCC3(C(F)(F)F)CC3)CC2)C1] | 428.4 [M + H]⁺ |
| 648 | [(3S)-3-(1H-1,2,4-Triazol-5- yl)pyrrolidin-1-yl]-[7-[[4- (trifluoromethylsulfonyl)phenyl] methyl]-2,7- diazaspiro[3.5]nonan-2- yl]methanone | 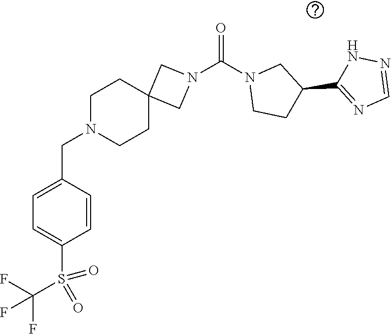 [SMILES: O=C(N1CC[C@H](c2ncn[nH]2)C1)N1CC2(CCN(Cc3ccc(S(=O)(=O)C(F)(F)F)cc3)CC2)C1] | 513.3 [M + H]⁺ |
| 649 | [(3S)-3-(1H-1,2,4-Triazol-5- yl)pyrrolidin-1-yl]-[7-[[3- (trifluoromethylsulfonyl)phenyl] methyl]-2,7- diazaspiro[3.5]nonan-2- yl]methanone | 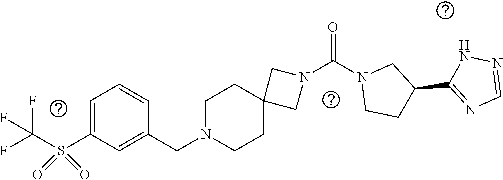 [SMILES: O=C(N1CC[C@H](c2ncn[nH]2)C1)N1CC2(CCN(Cc3cccc(S(=O)(=O)C(F)(F)F)c3)CC2)C1] | 513.3 [M + H]⁺ |
| 651 | [(3S)-3-(1H-1,2,4-Triazol-5- yl)pyrrolidin-1-yl]-[6-[[6- (trifluoromethyl)pyridazin-3- yl]methyl]-2- azaspiro[3.3]heptan-2- yl]methanone | 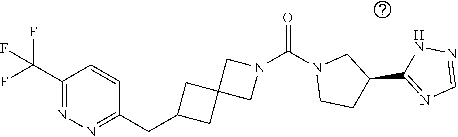 [SMILES: O=C(N1CC[C@H](c2ncn[nH]2)C1)N1CC2(CC(Cc3ccc(C(F)(F)F)nn3)C2)C1] | 422.3 [M + H]⁺ |
| 652 | [(3S)-3-(1H-1,2,4-Triazol-5- yl)pyrrolidin-1-yl]-[6-[[2- (trifluoromethyl)pyrimidin-5- yl]methyl]-2- azaspiro[3.3]heptan-2- yl]methanone | 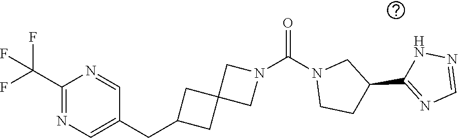 [SMILES: O=C(N1CC[C@H](c2ncn[nH]2)C1)N1CC2(CC(Cc3cnc(C(F)(F)F)nc3)C2)C1] | 422.3 [M + H]⁺ |
| 655 | [6-[[3-Fluoro-5- (trifluoromethyl)phenyl]methyl]- 2-azaspiro[3.3]heptan-2-yl]- [(3S)-3-(1H-1,2,4-triazol-5- yl)pyrrolidin-1-yl]methanone | 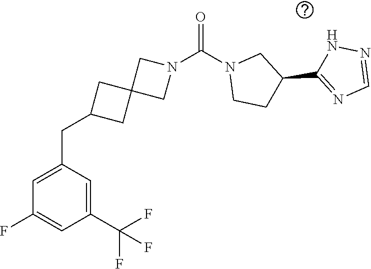 [SMILES: O=C(N1CC[C@H](c2ncn[nH]2)C1)N1CC2(CC(Cc3cc(F)cc(C(F)(F)F)c3)C2)C1] | 438.3 [M + H]⁺ |
| 656 | [(3S)-3-(1H-1,2,4-Triazol-5- yl)pyrrolidin-1-yl]-[(6S)-6-[[5- (trifluoromethyl)pyrazin-2- yl]methyl]-2-azaspiro[3.4]octan- 2-yl]methanone | 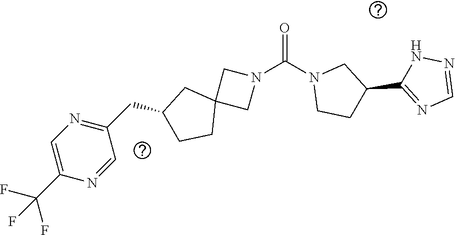 [SMILES: O=C(N1CC[C@H](c2ncn[nH]2)C1)N1CC2(CC[C@H](Cc3cnc(C(F)(F)F)cn3)C2)C1] | 436.3 [M + H]⁺ |
| 657 | [(3S)-3-(1H-1,2,4-Triazol-5- yl)pyrrolidin-1-yl]-[(6R)-6-[[5- (trifluoromethyl)pyrazin-2- yl]methyl]-2-azaspiro[3.4]octan- 2-yl]methanone | 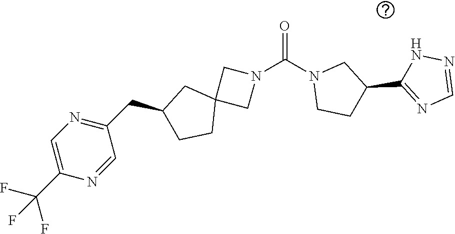 [SMILES: O=C(N1CC[C@H](c2ncn[nH]2)C1)N1CC2(CC[C@@H](Cc3cnc(C(F)(F)F)cn3)C2)C1] | 436.3 [M + H]⁺ |
| 658 | [6-[(4- Methylsulfonylphenyl)methyl]- 2-azaspiro[3.4]octan-2-yl]-[rac- (3S)-3-(1H-1,2,4-triazol-5- yl)pyrrolidin-1-yl]methanone | 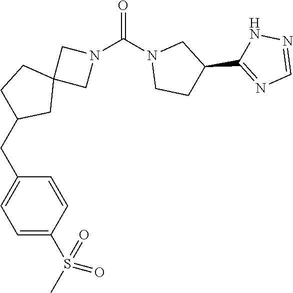 [SMILES: CS(=O)(=O)c1ccc(CC2CCC3(C2)CN(C(=O)N2CC[C@H](c4ncn[nH]4)C2)C3)cc1] | 444.4 [M + H]⁺ |
| 659 | [(3S)-3-(1H-1,2,4-Triazol-5- yl)pyrrolidin-1-yl]-[(6S)-6-[5- (trifluoromethyl)pyrazin-2- yl]oxy-2-azaspiro[3.4]octan-2- yl]methanone | 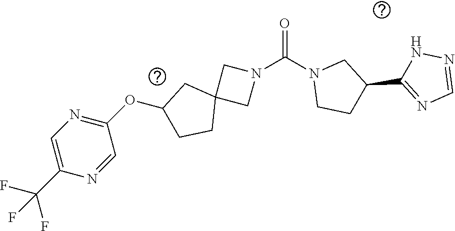 [SMILES: O=C(N1CC[C@H](c2ncn[nH]2)C1)N1CC2(CCC(Oc3cnc(C(F)(F)F)cn3)C2)C1] | 438.3 [M + H]⁺ |
| 660 | [(3S)-3-(1H-1,2,4-Triazol-5- yl)pyrrolidin-1-yl]-[3-[1-[[4- (trifluoromethyl)phenyl]methyl] azetidin-3-yl]azetidin-1- yl]methanone | 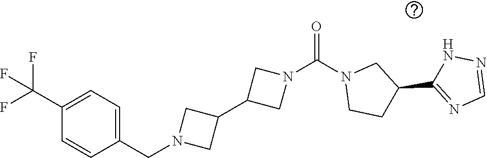 [SMILES: O=C(N1CC(C2CN(Cc3ccc(C(F)(F)F)cc3)C2)C1)N1CC[C@H](c2ncn[nH]2)C1] | 435.4 [M + H]⁺ |
| 661 | [(3S)-3-(1H-1,2,4-Triazol-5- yl)pyrrolidin-1-yl]-[6-[[5- (trifluoromethyl)-3- pyridyl]methyl]-2- azaspiro[3.3]heptan-2- yl]methanone | 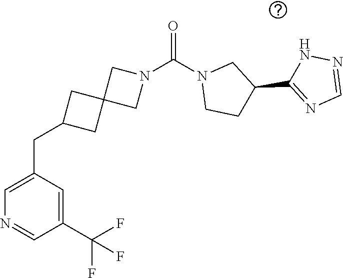 [SMILES: O=C(N1CC[C@H](c2ncn[nH]2)C1)N1CC2(CC(Cc3cncc(C(F)(F)F)c3)C2)C1] | 421.4 [M + H]⁺ |
| 662 | [(3S)-3-(1H-1,2,4-Triazol-5- yl)pyrrolidin-1-yl]-[6-[[4- (trifluoromethyl)-2- pyridyl]methyl]-2- azaspiro[3.3]heptan-2- yl]methanone | 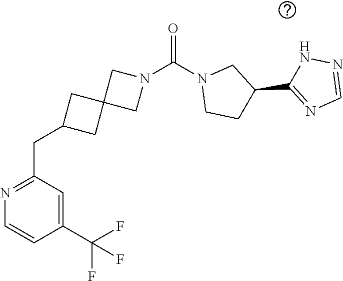 [SMILES: O=C(N1CC[C@H](c2ncn[nH]2)C1)N1CC2(CC(Cc3cc(C(F)(F)F)ccn3)C2)C1] | 421.4 [M + H]⁺ |
| 663 | [(3S)-3-(1H-1,2,4-Triazol-5- yl)pyrrolidin-1-yl]-[7-[[5- (trifluoromethyl)-2- pyridyl]methyl]-2,7- diazaspiro[3.4]octan-2- yl]methanone | 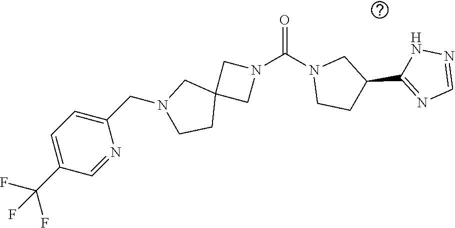 [SMILES: O=C(N1CC[C@H](c2ncn[nH]2)C1)N1CC2(CCN(Cc3ccc(C(F)(F)F)cn3)C2)C1] | 436.4 [M + H]⁺ |
| 664 | [(3S)-3-(1H-1,2,4-Triazol-5- yl)pyrrolidin-1-yl]-[3-[3-[[1- (trifluoromethyl)cyclopropyl] methylamino]-1- bicyclo[1.1.1]pentanyl]azetidin- 1-yl]methanone | 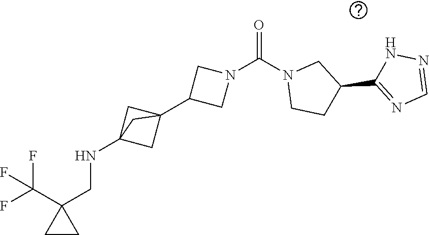 [SMILES: O=C(N1CC(C23CC(NCC4(C(F)(F)F)CC4)(C2)C3)C1)N1CC[C@H](c2ncn[nH]2)C1] | 425.4 [M + H]⁺ |
| 665 | [(3S)-3-(1H-1,2,4-Triazol-5- yl)pyrrolidin-1-yl]-[3-[3-[[[1- (trifluoromethyl)cyclopropyl] amino]methyl]-1- bicyclo[1.1.1]pentanyl]azetidin- 1-yl]methanone | 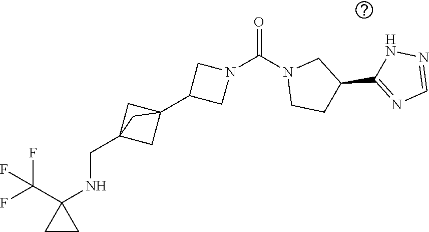 [SMILES: O=C(N1CC(C23CC(CNC4(C(F)(F)F)CC4)(C2)C3)C1)N1CC[C@H](c2ncn[nH]2)C1] | 425.4 [M + H]⁺ |
| 666 | [3-[3-(5-Cyclopropyl-1,3,4- oxadiazol-2-yl)-1- bicyclo[1.1.1]pentanyl]azetidin- 1-yl]-[(3S)-3-(1H-1,2,4-triazol- 5-yl)pyrrolidin-1-yl]methanone | 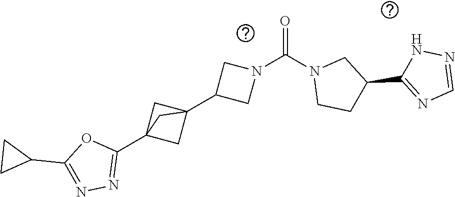 [SMILES: O=C(N1CC(C23CC(c4nnc(C5CC5)o4)(C2)C3)C1)N1CC[C@H](c2ncn[nH]2)C1] | 396.4 [M + H]⁺ |
| 667 | [(3S)-3-(1H-1,2,4-Triazol-5- yl)pyrrolidin-1-yl]-[3-[3-[5- (2,2,2-trifluoroethyl)-1,3,4- oxadiazol-2-yl]-1- bicyclo[1.1.1]pentanyl]azetidin- 1-yl]methanone | 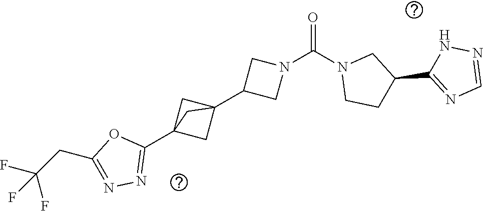 [SMILES: O=C(N1CC(C23CC(c4nnc(CC(F)(F)F)o4)(C2)C3)C1)N1CC[C@H](c2ncn[nH]2)C1] | 438.4 [M + H]⁺ |
| 668 | (4S)-1-[4-[1-[(3S)-3-(1H-1,2,4- Triazol-5-yl)pyrrolidine-1- carbonyl]azetidin-3-yl]phenyl]- 4-(trifluoromethyl)piperidin-2- one | 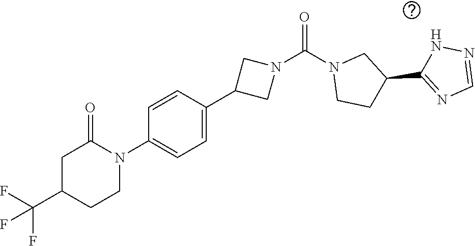 [SMILES: O=C(N1CC(c2ccc(N3CCC(C(F)(F)F)CC3=O)cc2)C1)N1CC[C@H](c2ncn[nH]2)C1] | 463.3 [M + H]⁺ |
| 669 | (4R)-1-[4-[1-[(3S)-3-(1H-1,2,4- Triazol-5-yl)pyrrolidine-1- carbonyl]azetidin-3-yl]phenyl]- 4-(trifluoromethyl)piperidin-2- one | 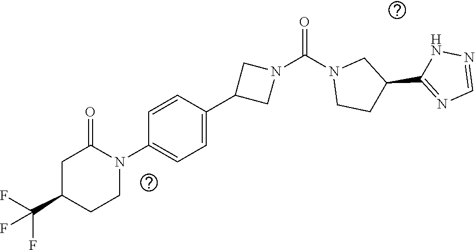 [SMILES: O=C(N1CC(c2ccc(N3CC[C@@H](C(F)(F)F)CC3=O)cc2)C1)N1CC[C@H](c2ncn[nH]2)C1] | 463.4 [M + H]⁺ |
| 672 | [3-[4-(3-tert-Butyl-5-methyl- pyrazol-1-yl)phenyl]azetidin-1- yl]-[(3S)-3-(1H-1,2,4-triazol-5- yl)pyrrolidin-1-yl]methanone | 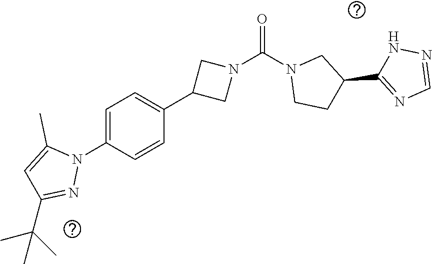 [SMILES: Cc1cc(C(C)(C)C)nn1-c1ccc(C2CN(C(=O)N3CC[C@H](c4ncn[nH]4)C3)C2)cc1] | 432.5 [M − H]⁻ |
| 674 | (3S)-1-[7-[[5-(Trifluoromethyl)- 2-pyridyl]methyl]-2,7- diazaspiro[3.4]octane-2- carbonyl]pyrrolidine-3- carboxamide | 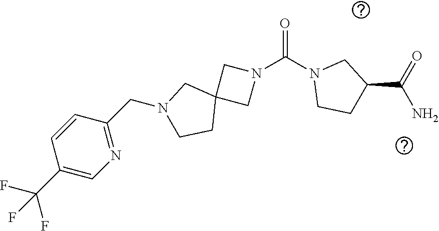 [SMILES: NC(=O)[C@H]1CCN(C(=O)N2CC3(CCN(Cc4ccc(C(F)(F)F)cn4)C3)C2)C1] | 412.3 [M + H]⁺ |
| 675 | (3S)-1-[3-[3-[5-(2,2,2- Trifluoroethyl)-1,3,4-oxadiazol- 2-yl]-1- bicyclo[1.1.1]pentanyl]azetidine- 1-carbonyl]pyrrolidine-3- carboxamide | 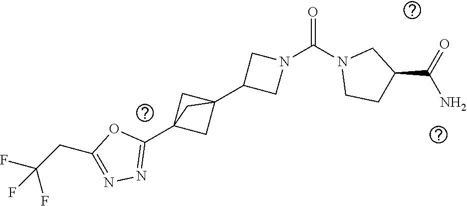 [SMILES: NC(=O)[C@H]1CCN(C(=O)N2CC(C34CC(c5nnc(CC(F)(F)F)o5)(C3)C4)C2)C1] | 414.3 [M + H]⁺ |
| 676 | [6-[[4-Methylsulfonyl-3- (trifluoromethyl)phenyl]methyl]- 2-azaspiro[3.3]heptan-2-yl]- [(3S)-3-(1H-1,2,4-triazol-5- yl)pyrrolidin-1-yl]methanone | 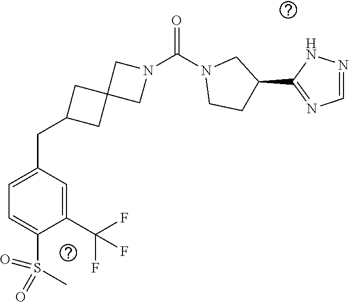 [SMILES: CS(=O)(=O)c1ccc(CC2CC3(C2)CN(C(=O)N2CC[C@H](c4ncn[nH]4)C2)C3)cc1C(F)(F)F] | 498.4 [M + H]⁺ |
| 677 | [6-[[2-Methylsulfonyl-4- (trifluoromethyl)phenyl]methyl]- 2-azaspiro[3.3]heptan-2-yl]- [(3S)-3-(1H-1,2,4-triazol-5- yl)pyrrolidin-1-yl]methanone | 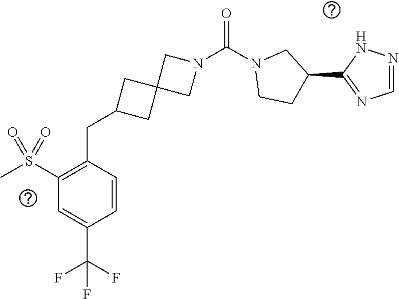 [SMILES: CS(=O)(=O)c1cc(C(F)(F)F)ccc1CC1CC2(C1)CN(C(=O)N1CC[C@H](c3ncn[nH]3)C1)C2] | 498.4 [M + H]⁺ |
| 678 | [6-[[3-Methylsulfonyl-4- (trifluoromethyl)phenyl]methyl]- 2-azaspiro[3.3]heptan-2-yl]- [(3S)-3-(1H-1,2,4-triazol-5- yl)pyrrolidin-1-yl]methanone | 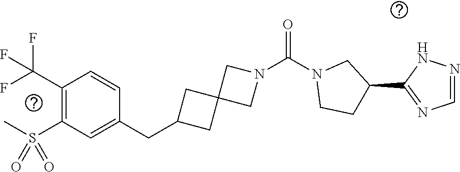 [SMILES: CS(=O)(=O)c1cc(CC2CC3(C2)CN(C(=O)N2CC[C@H](c4ncn[nH]4)C2)C3)ccc1C(F)(F)F] | 498.4 [M + H]⁺ |
| 680 | [rac-(3S)-3-(1H-1,2,4-Triazol-5- yl)pyrrolidin-1-yl]-[6-[[4- (trifluoromethylsulfonimidoyl) phenyl]methyl]-2- azaspiro[3.3]heptan-2- yl]methanone | 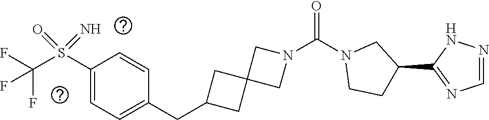 [SMILES: N=S(=O)(c1ccc(CC2CC3(C2)CN(C(=O)N2CC[C@H](c4ncn[nH]4)C2)C3)cc1)C(F)(F)F] | 483.4 [M + H]⁺ |
| 681 | [(3S)-3-(1H-1,2,4-Triazol-5- yl)pyrrolidin-1-yl]-[6-[[4- (trifluoromethyl)pyrazol-1- yl]methyl]-2- azaspiro[3.3]heptan-2- yl]methanone | 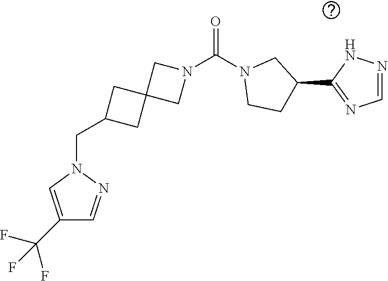 [SMILES: O=C(N1CC[C@H](c2ncn[nH]2)C1)N1CC2(CC(Cn3cc(C(F)(F)F)cn3)C2)C1] | 410.3 [M + H]⁺ |
| 682 | [(3S)-3-(1H-1,2,4-Triazol-5- yl)pyrrolidin-1-yl]-[3-[4-[5-[1- (trifluoromethyl)cyclopropyl]- 4H-1,2,4-triazol-3- yl]phenyl]azetidin-1- yl]methanone | 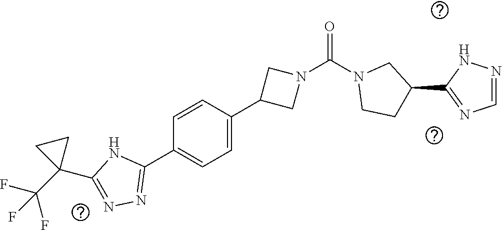 [SMILES: O=C(N1CC(c2ccc(-c3nnc(C4(C(F)(F)F)CC4)[nH]3)cc2)C1)N1CC[C@H](c2ncn[nH]2)C1] | 473.4 [M + H]⁺ |
| 683 | (3S)-1-[3-[3-[[1- (Trifluoromethyl)cyclopropyl] methylamino]-1- bicyclo[1.1.1]pentanyl]azetidine- 1-carbonyl]pyrrolidine-3- carboxamide | 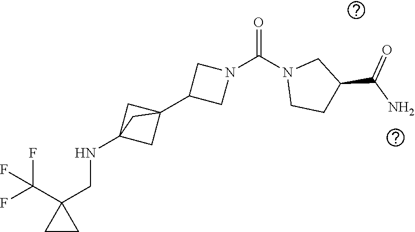 [SMILES: NC(=O)[C@H]1CCN(C(=O)N2CC(C34CC(NCC5(C(F)(F)F)CC5)(C3)C4)C2)C1] | 401.4 [M + H]⁺ |
| 684 | (3S)-1-[3-[3-[[[1- (Trifluoromethyl)cyclopropyl] amino]methyl]-1- bicyclo[1.1.1]pentanyl]azetidine- 1-carbonyl]pyrrolidine-3- carboxamide | 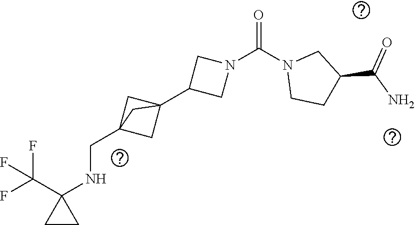 [SMILES: NC(=O)[C@H]1CCN(C(=O)N2CC(C34CC(CNC5(C(F)(F)F)CC5)(C3)C4)C2)C1] | 401.4 [M + H]⁺ |
| 685 | rac-(1S,4R,5R)-2-[3-[4-(4- Chloro-2-methylsulfonyl- phenyl)phenyl]azetidine-1- carbonyl]-2- azabicyclo[2.1.1]hexane-5- carboxamide | 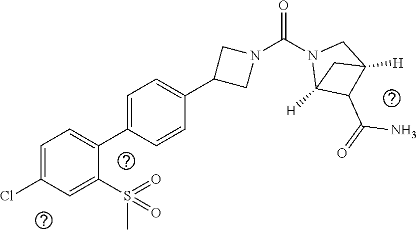 [SMILES: CS(=O)(=O)c1cc(Cl)ccc1-c1ccc(C2CN(C(=O)N3C[C@@H]4C[C@H]3C4C(N)=O)C2)cc1] | 474.3 [M + H]⁺ |
| 686 | (3S)-1-[3-[3-(5-Cyclopropyl- 1,3,4-oxadiazol-2-yl)-1- bicyclo[1.1.1]pentanyl]azetidine- 1-carbonyl]pyrrolidine-3- carboxamide | 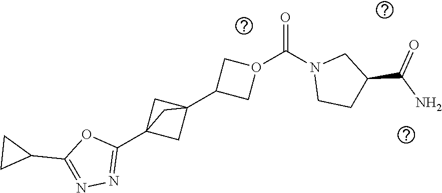 | 372.3 [M + H]⁺ |
| 687 | (3S)-1-[7-(4-Chloro-2- methylsulfonyl-phenyl)-2,7- diazaspiro[3.5]nonane-2- carbonyl]pyrrolidine-3- carboxamide | 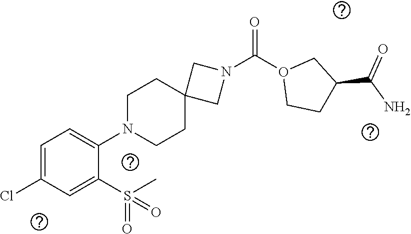 | 455.3 [M + H]⁺ |
| 688 | [6-[(5-Methylsulfonyl-2- pyridyl)methyl]-2- azaspiro[3.3]heptan-2-yl]-[(3S)- 3-(1H-1,2,4-triazol-5- yl)pyrrolidin-1-yl]methanone | 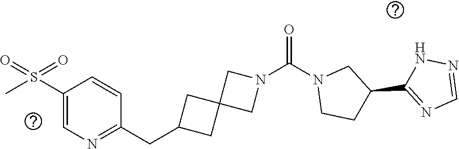 [SMILES: CS(=O)(=O)c1ccc(CC2CC3(C2)CN(C(=O)N2CC[C@H](c4ncn[nH]4)C2)C3)nc1] | 431.4 [M + H]⁺ |
| 690 | [(3S)-3-(1H-1,2,4-Triazol-5- yl)pyrrolidin-1-yl]-[6-[[3- (trifluoromethyl)-1,2,4-triazol-1- yl]methyl]-2- azaspiro[3.3]heptan-2- yl]methanone | 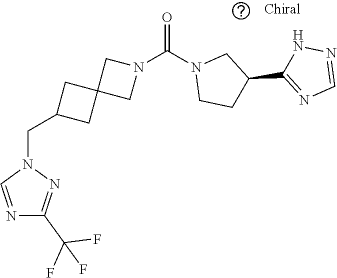 [SMILES: O=C(N1CC[C@H](c2ncn[nH]2)C1)N1CC2(CC(Cn3cnc(C(F)(F)F)n3)C2)C1] | 411.4 [M + H]⁺ |
| 691 | [(3S)-3-(1H-1,2,4-Triazol-5- yl)pyrrolidin-1-yl]-[6-[[4- (trifluoromethyl)oxazol-2- yl]methyl]-2- azaspiro[3.3]heptan-2- yl]methanone | 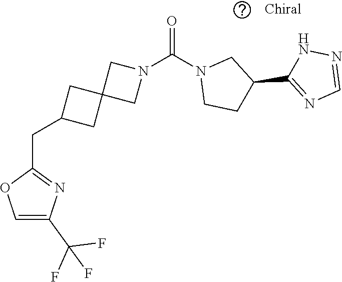 [SMILES: O=C(N1CC[C@H](c2ncn[nH]2)C1)N1CC2(CC(Cc3nc(C(F)(F)F)co3)C2)C1] | 411.4 [M + H]⁺ |
| 692 | [(3S)-3-(1H-1,2,4-Triazol-5- yl)pyrrolidin-1-yl]-[6-[[3- (trifluoromethyl)-1,2,4- thiadiazol-5-yl]methyl]-2- azaspiro[3.3]heptan-2- yl]methanone | 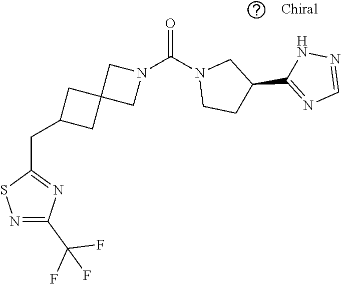 [SMILES: O=C(N1CC[C@H](c2ncn[nH]2)C1)N1CC2(CC(Cc3nc(C(F)(F)F)ns3)C2)C1] | 428.3 [M + H]⁺ |
| 693 | [(3S)-3-(1H-1,2,4-Triazol-5- yl)pyrrolidin-1-yl]-[3-[3-[5- (trifluoromethyl)pyrazin-2- yl]oxy-1- bicyclo[1.1.1]pentanyl]azetidin- 1-yl]methanone | 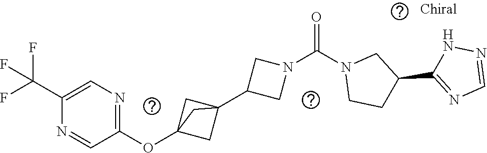 [SMILES: O=C(N1CC(C23CC(Oc4cnc(C(F)(F)F)cn4)(C2)C3)C1)N1CC[C@H](c2ncn[nH]2)C1] | 450.4 [M + H]⁺ |
| 694 | [(3S)-3-(1H-1,2,4-Triazol-5- yl)pyrrolidin-1-yl]-[6-[[4- (trifluoromethyl)imidazol-1- yl]methyl]-2- azaspiro[3.3]heptan-2- yl]methanone | 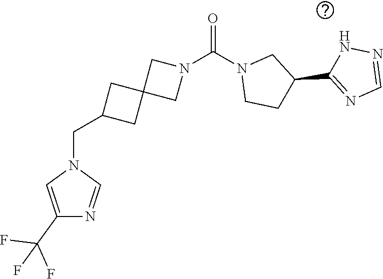 [SMILES: O=C(N1CC[C@H](c2ncn[nH]2)C1)N1CC2(CC(Cn3cnc(C(F)(F)F)c3)C2)C1] | 410.4 [M + H]⁺ |
| 696 | [3-[4-(3,5-Dimethyl-1,2,4- triazol-1-yl)phenyl]azetidin-1- yl]-[(3S)-3-(1H-1,2,4-triazol-5- yl)pyrrolidin-1-yl]methanone | 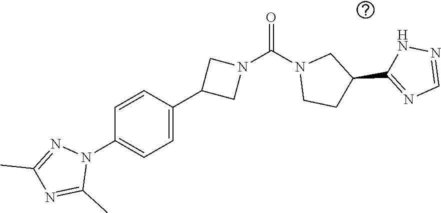 [SMILES: Cc1nc(C)n(-c2ccc(C3CN(C(=O)N4CC[C@H](c5ncn[nH]5)C4)C3)cc2)n1] | 393.4 [M + H]⁺ |
| 698 | [6-[(3-Fluoro-5-methylsulfonyl- phenyl)methyl]-2- azaspiro[3.3]heptan-2-yl]-[(3S)- 3-(4H-1,2,4-triazol-3- yl)pyrrolidin-1-yl]methanone | 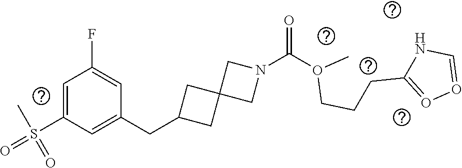 | 448.3 [M + H]⁺ |
| 699 | [6-[[4-Methylsulfonyl-3- (trifluoromethyl)phenyl]methyl]- 2-azaspiro[3.4]octan-2-yl]-[rac- (3S)-3-(4H-1,2,4-triazol-3- yl)pyrrolidin-1-yl]methanone | 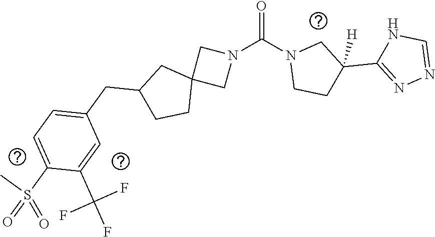 [SMILES: CS(=O)(=O)c1ccc(CC2CCC3(C2)CN(C(=O)N2CC[C@H](c4nnc[nH]4)C2)C3)cc1C(F)(F)F] | 512.3 [M + H]⁺ |
| 702 | [3-[6-(2-Chlorophenoxy)-3- pyridyl]azetidin-1-yl]-[(3S)-3- (4H-1,2,4-triazol-3- yl)pyrrolidin-1-yl]methanone | 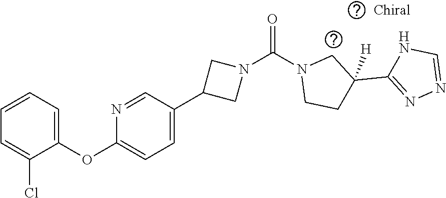 [SMILES: O=C(N1CC(c2ccc(Oc3ccccc3Cl)nc2)C1)N1CC[C@H](c2nnc[nH]2)C1] | 425.3 [M + H]⁺ |
| 703 | [6-[(3,5-Difluoro-2- pyridyl)methyl]-2- azaspiro[3.3]heptan-2-yl]-[(3S)- 3-(4H-1,2,4-triazol-3- yl)pyrrolidin-1-yl]methanone | 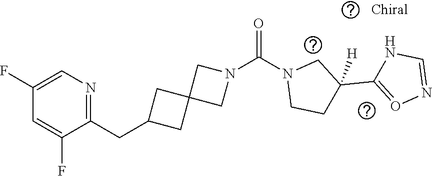 | 389.2 [M + H]⁺ |
| 704 | [7-[Methyl-[5- (trifluoromethyl)pyrazin-2- yl]amino]-2-azaspiro[3.5]nonan- 2-yl]-[(3S)-3-(4H-1,2,4-triazol- 3-yl)pyrrolidin-1-yl]methanone | 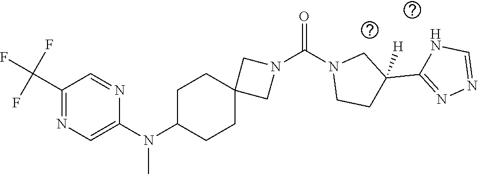 [SMILES: CN(c1cnc(C(F)(F)F)cn1)C1CCC2(CC1)CN(C(=O)N1CC[C@H](c3nnc[nH]3)C1)C2] | 465.4 [M + H]⁺ |
| 705 | [6-[2-Methylsulfonyl-4- (trifluoromethyl)phenyl]-2- azaspiro[3.3]heptan-2-yl]-[(3S)- 3-(4H-1,2,4-triazol-3- yl)pyrrolidin-1-yl]methanone | 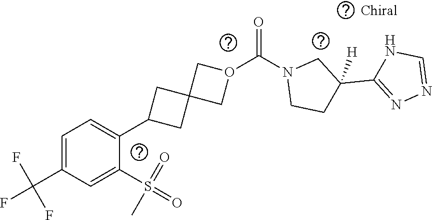 | 484.4 [M + H]⁺ |
| 706 | [(3S)-3-(4H-1,2,4-Triazol-3- yl)pyrrolidin-1-yl]-[3-[[4- (trifluoromethylsulfonyl)phenyl] methoxy]azetidin-1- yl]methanone | 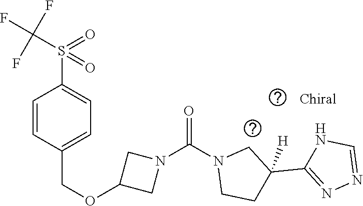 [SMILES: O=C(N1CC(OCc2ccc(S(=O)(=O)C(F)(F)F)cc2)C1)N1CC[C@H](c2nnc[nH]2)C1] | 460.4 [M + H]⁺ |
| 707 | [6-(4-Methylsulfonylphenyl)-2- azaspiro[3.3]heptan-2-yl]-[(3S)- 3-(4H-1,2,4-triazol-3- yl)pyrrolidin-1-yl]methanone | 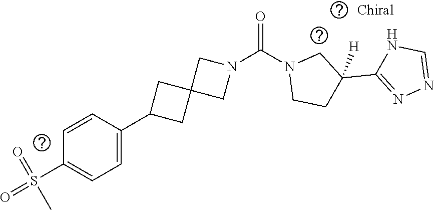 [SMILES: CS(=O)(=O)c1ccc(C2CC3(C2)CN(C(=O)N2CC[C@H](c4nnc[nH]4)C2)C3)cc1] | 416.4 [M + H]⁺ |
| 708 | [(3S)-3-(4H-1,2,4-Triazol-3- yl)pyrrolidin-1-yl]-[6-[4- (trifluoromethyl- sulfonyl)phenyl]- 2-azaspiro[3.3]heptan-2- yl]methanone | 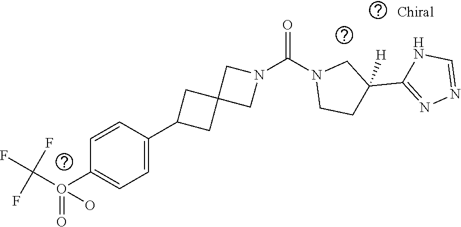 | 470.4 [M + H]⁺ |
| 709 | [(3S)-3-(4H-1,2,4-Triazol-3- yl)pyrrolidin-1-yl]-[3-[4-[2- (trifluoromethyl)pyrimidin-5- yl]oxyphenyl]azetidin-1- yl]methanone | 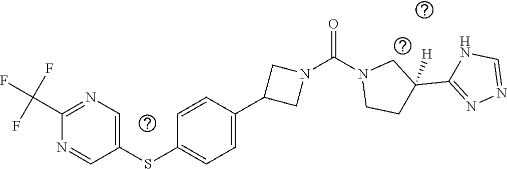 [SMILES: O=C(N1CC(c2ccc(Sc3cnc(C(F)(F)F)nc3)cc2)C1)N1CC[C@H](c2nnc[nH]2)C1] | 460.4 [M + H]⁺ |
| 710 | [(3S)-3-(4H-1,2,4-Triazol-3- yl)pyrrolidin-1-yl]-[7-[[5- (trifluoromethyl)pyrazin-2- yl]amino]-2-azaspiro[3.5]nonan- 2-yl]methanone | 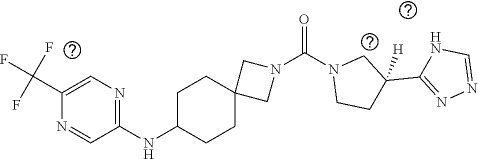 [SMILES: O=C(N1CC[C@H](c2nnc[nH]2)C1)N1CC2(CCC(Nc3cnc(C(F)(F)F)cn3)CC2)C1] | 451.3 [M + H]⁺ |
| 711 | [7-(5-Chloro-2-methylsulfonyl- phenoxy)-2-azaspiro[3.5]nonan- 2-yl]-[(3S)-3-(4H-1,2,4-triazol- 3-yl)pyrrolidin-1-yl]methanone | 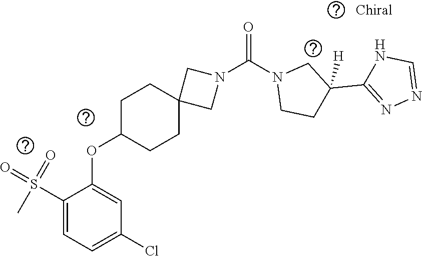 [SMILES: CS(=O)(=O)c1ccc(Cl)cc1OC1CCC2(CC1)CN(C(=O)N1CC[C@H](c3nnc[nH]3)C1)C2] | 494.3 [M + H]⁺ |
| 712 | [3-[4-(3,5-Dimethylpyrazol-1- yl)phenyl]azetidin-1-yl]-[(3S)-3- (4H-1,2,4-triazol-3- yl)pyrrolidin-1-yl]methanone | 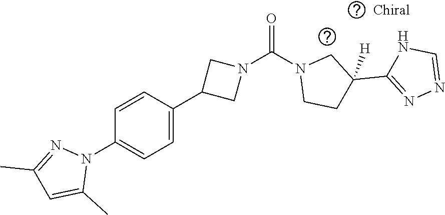 [SMILES: Cc1cc(C)n(-c2ccc(C3CN(C(=O)N4CC[C@H](c5nnc[nH]5)C4)C3)cc2)n1] | 392.3 [M + H]⁺ |
| 713 | [3-[6-(N-Methylanilino)-3- pyridyl]azetidin-1-yl]-[(3S)-3- (4H-1,2,4-triazol-3- yl)pyrrolidin-1-yl]methanone | 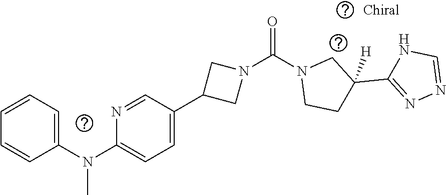 [SMILES: CN(c1ccccc1)c1ccc(C2CN(C(=O)N3CC[C@H](c4nnc[nH]4)C3)C2)cn1] | 404.3 [M + H]⁺ |
| 714 | 2-Methoxy-4-[[2-[(3S)-3-(4H- 1,2,4-triazol-3-yl)pyrrolidine-1- carbonyl]-2-azaspiro[3.3]heptan- 6-yl]methyl]benzonitrile | 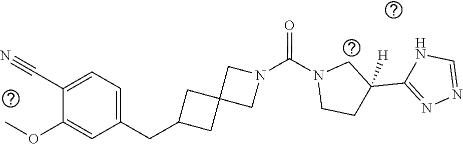 [SMILES: COc1cc(CC2CC3(C2)CN(C(=O)N2CC[C@H](c4nnc[nH]4)C2)C3)ccc1C#N] | 407.3 [M + H]⁺ |
| 716 | [3-[6-(4-Fluorophenoxy)-3- pyridyl]azetidin-1-yl]-[(3S)-3- (4H-1,2,4-triazol-3- yl)pyrrolidin-1-yl]methanone | 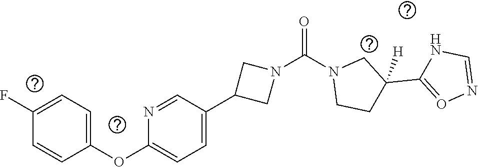 | 409.3 [M + H]⁺ |
| 717 | [3-(4-Cyclopropyl-2-fluoro- phenoxy)azetidin-1-yl]-[(3S)-3- (4H-1,2,4-triazol-3- yl)pyrrolidin-1-yl]methanone | 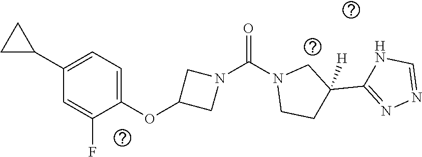 [SMILES: O=C(N1CC(Oc2ccc(C3CC3)cc2F)C1)N1CC[C@H](c2nnc[nH]2)C1] | 372.2 [M + H]⁺ |
| 718 | [3-(4-Chloro-3-cyclopropyl- phenoxy)azetidin-1-yl]-[(3S)-3- (4H-1,2,4-triazol-3- yl)pyrrolidin-1-yl]methanone | 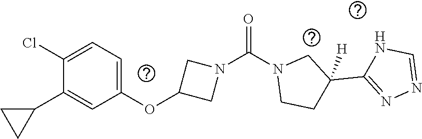 [SMILES: O=C(N1CC(Oc2ccc(Cl)c(C3CC3)c2)C1)N1CC[C@H](c2nnc[nH]2)C1] | 388.2 [M + H]⁺ |
| 719 | [3-[4-[5-[(1- Methylcyclopropyl)methyl]-4H- 1,2,4-triazol-3- yl]phenyl]azetidin-1-yl]-[(3S)-3- (4H-1,2,4-triazol-3- yl)pyrrolidin-1-yl]methanone | 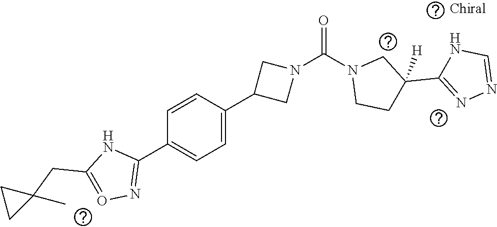 | 433.4 [M + H]⁺ |
| 720 | [(3S)-3-(1H-1,2,4-Triazol-5- yl)pyrrolidin-1-yl]-[6-[[4- (trifluoromethyl)thiazol-2- yl]methyl]-2- azaspiro[3.3]heptan-2- yl]methanone | 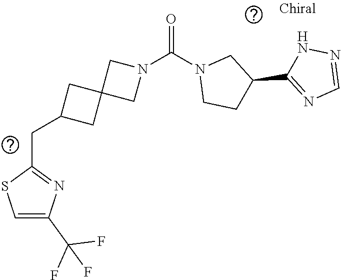 [SMILES: O=C(N1CC[C@H](c2ncn[nH]2)C1)N1CC2(CC(Cc3nc(C(F)(F)F)cs3)C2)C1] | 427.3 [M + H]⁺ |
| 721 | (3S)-1-[6-[[4- (Trifluoromethyl)pyrazol-1- yl]methyl]-2- azaspiro[3.3]heptane-2- carbonyl]pyrrolidine-3- carboxamide | 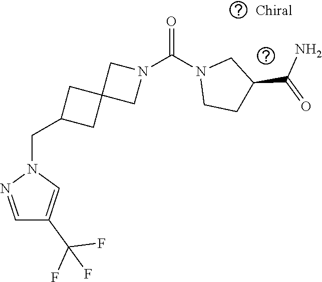 [SMILES: NC(=O)[C@H]1CCN(C(=O)N2CC3(CC(Cn4cc(C(F)(F)F)cn4)C3)C2)C1] | 386.3 [M + H]⁺ |
| 722 | (3S)-1-[6-[[4- (Trifluoromethyl)imidazol-1- yl]methyl]-2- azaspiro[3.3]heptane-2- carbonyl]pyrrolidine-3- carboxamide | 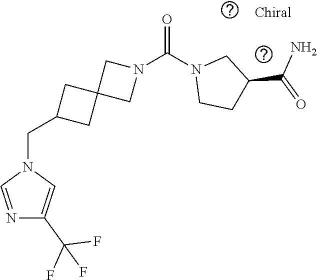 [SMILES: NC(=O)[C@H]1CCN(C(=O)N2CC3(CC(Cn4cnc(C(F)(F)F)c4)C3)C2)C1] | 386.3 [M + H]⁺ |
| 723 | (3S)-1-[6-[[3-(Trifluoromethyl)- 1,2,4-triazol-1-yl]methyl]-2- azaspiro[3.3]heptane-2- carbonyl]pyrrolidine-3- carboxamide | 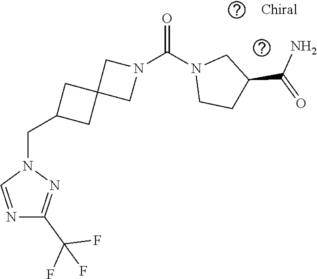 [SMILES: NC(=O)[C@H]1CCN(C(=O)N2CC3(CC(Cn4cnc(C(F)(F)F)n4)C3)C2)C1] | 387.3 [M + H]⁺ |
| 724 | (3S)-1-[6-[[4- (Trifluoromethyl)oxazol-2- yl]methyl]-2- azaspiro[3.3]heptane-2- carbonyl]pyrrolidine-3- carboxamide | 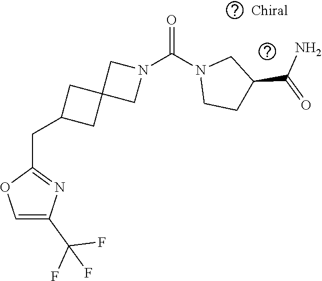 [SMILES: NC(=O)[C@H]1CCN(C(=O)N2CC3(CC(Cc4nc(C(F)(F)F)co4)C3)C2)C1] | 387.3 [M + H]⁺ |
| 725 | (3S)-1-[6-[[3-(Trifluoromethyl)- 1,2,4-thiadiazol-5-yl]methyl]-2- azaspiro[3.3]heptane-2- carbonyl]pyrrolidine-3- carboxamide | 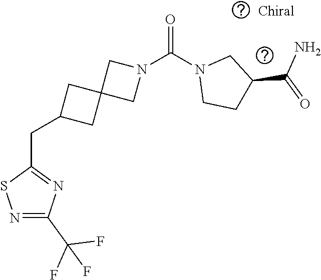 [SMILES: NC(=O)[C@H]1CCN(C(=O)N2CC3(CC(Cc4nc(C(F)(F)F)ns4)C3)C2)C1] | 404.3 [M + H]⁺ |
| 726 | (3S)-1-[6-[[4- (Trifluoromethyl)thiazol-2- yl]methyl]-2- azaspiro[3.3]heptane-2- carbonyl]pyrrolidine-3- carboxamide | 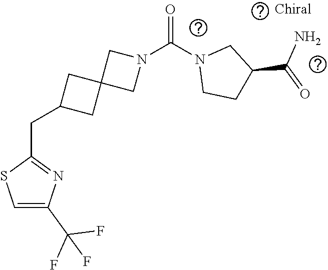 [SMILES: NC(=O)[C@H]1CCN(C(=O)N2CC3(CC(Cc4nc(C(F)(F)F)cs4)C3)C2)C1] | 403.3 [M + H]⁺ |
| 727 | [6-[(5-Fluoro-3-pyridyl)methyl]- 2-azaspiro[3.3]heptan-2-yl]- [(3S)-3-(4H-1,2,4-triazol-3- yl)pyrrolidin-1-yl]methanone | 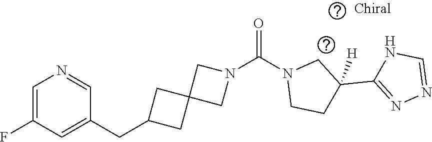 [SMILES: O=C(N1CC[C@H](c2nnc[nH]2)C1)N1CC2(CC(Cc3cncc(F)c3)C2)C1] | 371.2 [M + H]⁺ |
| 728 | [6-[(2-Fluoro-4-methylsulfonyl- phenyl)methyl]-2- azaspiro[3.3]heptan-2-yl]-[(3S)- 3-(4H-1,2,4-triazol-3- yl)pyrrolidin-1-yl]methanone | 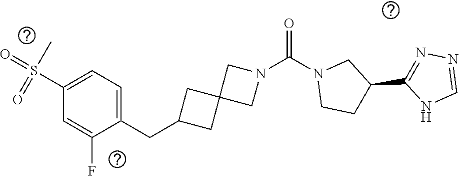 [SMILES: CS(=O)(=O)c1ccc(CC2CC3(C2)CN(C(=O)N2CC[C@H](c4nnc[nH]4)C2)C3)c(F)c1] | 448.2 [M + H]⁺ |
| 729 | [6-([1,2,4]Triazolo[1,5- a]pyridin-7-ylmethyl)-2- azaspiro[3.3]heptan-2-yl]-[(3S)- 3-(1H-1,2,4-triazol-5- yl)pyrrolidin-1-yl]methanone | 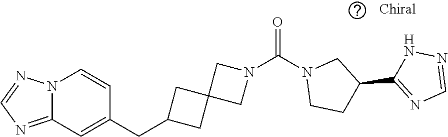 [SMILES: O=C(N1CC[C@H](c2ncn[nH]2)C1)N1CC2(CC(Cc3ccn4ncnc4c3)C2)C1] | 393.4 [M + H]⁺ |
| 730 | (3S)-1-[6-([1,2,4]Triazolo[1,5- a]pyridin-6-ylmethyl)-2- azaspiro[3.3]heptane-2- carbonyl]pyrrolidine-3- carboxamide | 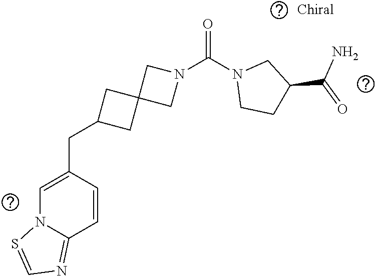 | 369.3 [M + H]⁺ |
| 731 | [6-(1H-Pyrazolo[4,3-b]pyridin- 5-ylmethyl)-2- azaspiro[3.3]heptan-2-yl]-[(3S)- 3-(1H-1,2,4-triazol-5- yl)pyrrolidin-1-yl]methanone | 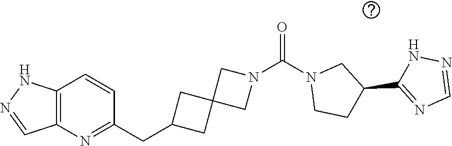 [SMILES: O=C(N1CC[C@H](c2ncn[nH]2)C1)N1CC2(CC(Cc3ccc4[nH]ncc4n3)C2)C1] | 393.3 [M + H]⁺ |
| 732 | [(3S)-3-(1H-1,2,4-Triazol-5- yl)pyrrolidin-1-yl]-[6-[[1- (trifluoromethyl)pyrazol-4- yl]methyl]-2- azaspiro[3.3]heptan-2- yl]methanone | 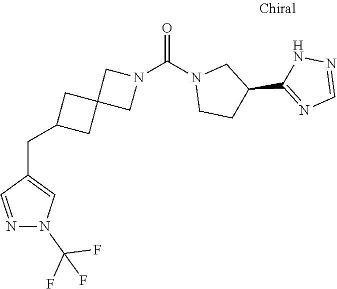 [SMILES: O=C(N1CC[C@H](c2ncn[nH]2)C1)N1CC2(CC(Cc3cnn(C(F)(F)F)c3)C2)C1] | 410.3 [M + H]⁺ |
| 733 | 3-[[2-[(3S)-3-(1H-1,2,4-Triazol- 5-yl)pyrrolidine-1-carbonyl]-2- azaspiro[3.3]heptan-6- yl]methyl]-5- (trifluoromethyl)benzonitrile | 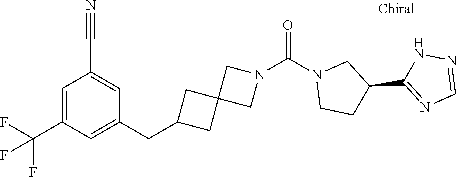 [SMILES: N#Cc1cc(CC2CC3(C2)CN(C(=O)N2CC[C@H](c4ncn[nH]4)C2)C3)cc(C(F)(F)F)c1] | 445.3 [M + H]⁺ |
| 737 | [6-[(4- Cyclopropylsulfonylphenyl) methyl]-2-azaspiro[3.3]heptan-2- yl]-[(3S)-3-(1H-1,2,4-triazol-5- yl)pyrrolidin-1-yl]methanone | 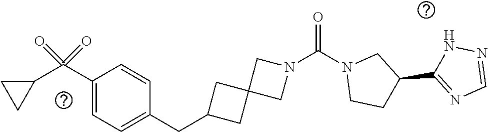 | 456.4 [M + H]⁺ |
| 738 | [(3S)-3-(1H-1,2,4-Triazol-5- yl)pyrrolidin-1-yl]-[3-[3-[[6- (trifluoromethyl)-3- pyridyl]methyl]-1- bicyclo[1.1.1]pentanyl]azetidin- 1-yl]methanone | 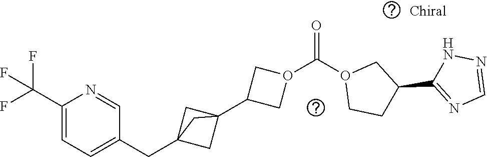 | 447.3 [M + H]⁺ |
| 739 | [6-[[2-Fluoro-4- (trifluoromethyl)phenyl]methyl]- 2-azaspiro[3.3]heptan-2-yl]-[3- (1H-1,2,4-triazol-5-yl)azetidin- 1-yl]methanone | 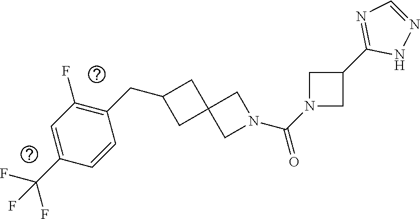 [SMILES: O=C(N1CC(c2ncn[nH]2)C1)N1CC2(CC(Cc3ccc(C(F)(F)F)cc3F)C2)C1] | 424.3 [M + H]⁺ |
| 740 | [6-[(2,4- Difluorophenyl)methyl]-2- azaspiro[3.3]heptan-2-yl]-[3- (1H-1,2,4-triazol-5-yl)azetidin- 1-yl]methanone | 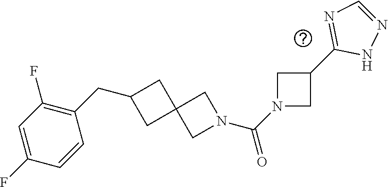 [SMILES: O=C(N1CC(c2ncn[nH]2)C1)N1CC2(CC(Cc3ccc(F)cc3F)C2)C1] | 374.3 [M + H]⁺ |
| 741 | [6-[[4-Fluoro-2- (trifluoromethyl)phenyl]methyl]- 2-azaspiro[3.3]heptan-2-yl]-[3- (1H-1,2,4-triazol-5-yl)azetidin- 1-yl]methanone | 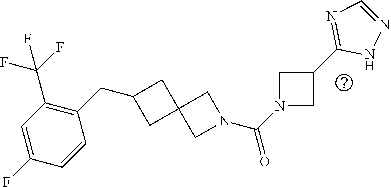 [SMILES: O=C(N1CC(c2ncn[nH]2)C1)N1CC2(CC(Cc3ccc(F)cc3C(F)(F)F)C2)C1] | 424.3 [M + H]⁺ |
| 742 | [6-[[3-Fluoro-5- (trifluoromethyl)phenyl]methyl]- 2-azaspiro[3.3]heptan-2-yl]-[3- (1H-1,2,4-triazol-5-yl)azetidin- 1-yl]methanone | 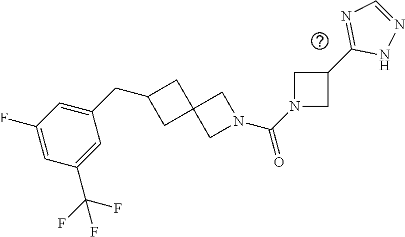 [SMILES: O=C(N1CC(c2ncn[nH]2)C1)N1CC2(CC(Cc3cc(F)cc(C(F)(F)F)c3)C2)C1] | 424.3 [M + H]⁺ |
| 743 | [6-[(3-Chloro-5-fluoro-2- pyridyl)methyl]-2- azaspiro[3.3]heptan-2-yl]-[3- (1H-1,2,4-triazol-5-yl)azetidin- 1-yl]methanone | 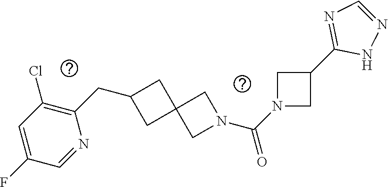 [SMILES: O=C(N1CC(c2ncn[nH]2)C1)N1CC2(CC(Cc3ncc(F)cc3Cl)C2)C1] | 391.2 [M + H]⁺ |
| 744 | [3-(1H-1,2,4-Triazol-5- yl)azetidin-1-yl]-[6-[[5- (trifluoromethoxy)-2- pyridyl]methyl]-2- azaspiro[3.3]heptan-2- yl]methanone | 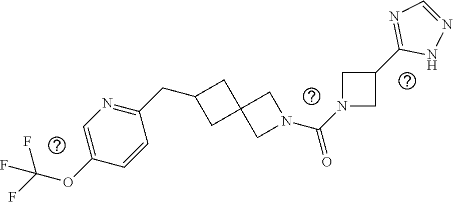 [SMILES: O=C(N1CC(c2ncn[nH]2)C1)N1CC2(CC(Cc3ccc(OC(F)(F)F)cn3)C2)C1] | 423.3 [M + H]⁺ |
| 745 | [6-[[3-Fluoro-5- (trifluoromethyl)-2- pyridyl]methyl]-2- azaspiro[3.3]heptan-2-yl]-[3- (1H-1,2,4-triazol-5-yl)azetidin- 1-yl]methanone | 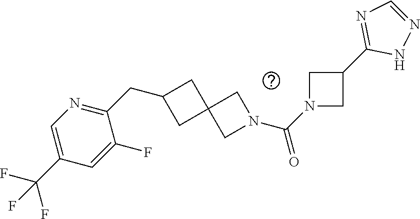 [SMILES: O=C(N1CC(c2ncn[nH]2)C1)N1CC2(CC(Cc3ncc(C(F)(F)F)cc3F)C2)C1] | 425.3 [M + H]⁺ |
| 746 | [3-(1H-1,2,4-Triazol-5- yl)azetidin-1-yl]-[7-[[5- (trifluoromethyl)-2- pyridyl]methyl]-2,7- diazaspiro[3.4]octan-2- yl]methanone | 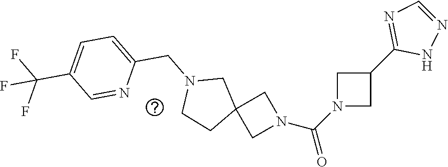 [SMILES: O=C(N1CC(c2ncn[nH]2)C1)N1CC2(CCN(Cc3ccc(C(F)(F)F)cn3)C2)C1] | 422.3 [M + H]⁺ |
| 747 | [7-[(5-Chloro-2-pyridyl)methyl]- 2-azaspiro[3.5]nonan-2-yl]-[3- (1H-1,2,4-triazol-5-yl)azetidin- 1-yl]methanone | 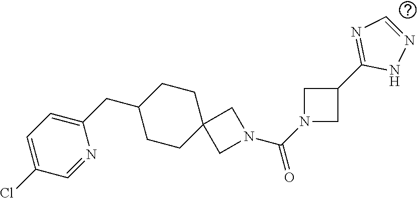 [SMILES: O=C(N1CC(c2ncn[nH]2)C1)N1CC2(CCC(Cc3ccc(Cl)cn3)CC2)C1] | 401.3 [M + H]⁺ |
| 748 | [7-[[6-(Difluoromethoxy)-3- pyridyl]methyl]-2- azaspiro[3.5]nonan-2-yl]-[3- (1H-1,2,4-triazol-5-yl)azetidin- 1-yl]methanone | 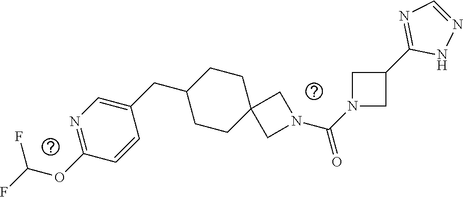 [SMILES: O=C(N1CC(c2ncn[nH]2)C1)N1CC2(CCC(Cc3ccc(OC(F)F)nc3)CC2)C1] | 433.3 [M + H]⁺ |
| 749 | [3-(1H-1,2,4-Triazol-5- yl)azetidin-1-yl]-[3-[4-[1- (trifluoromethyl)cyclopropyl] phenyl]azetidin-1-yl]methanone | 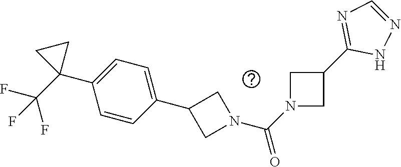 [SMILES: O=C(N1CC(c2ccc(C3(C(F)(F)F)CC3)cc2)C1)N1CC(c2ncn[nH]2)C1] | 392.3 [M + H]⁺ |
| 750 | [3-(1H-1,2,4-Triazol-5- yl)azetidin-1-yl]-[3-[4-[3- (trifluoromethyl)azetidin-1- yl]phenyl]azetidin-1- yl]methanone | 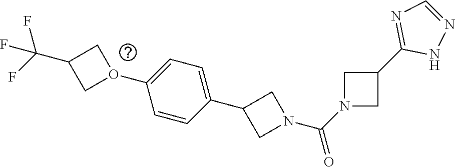 | 407.3 [M + H]⁺ |
| 751 | [3-[4-(4-Chloro-2- methylsulfonyl- phenyl)phenyl]azetidin-1-yl]-[3- (1H-1,2,4-triazol-5-yl)azetidin- 1-yl]methanone | 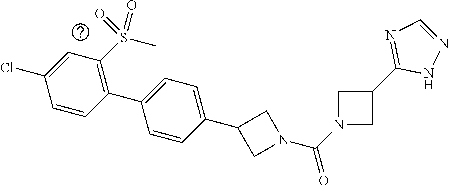 [SMILES: CS(=O)(=O)c1cc(Cl)ccc1-c1ccc(C2CN(C(=O)N3CC(c4ncn[nH]4)C3)C2)cc1] | 472.3 [M + H]⁺ |
| 752 | [3-[4-[5-[(1- Methylcyclopropyl)methyl]-4H- 1,2,4-triazol-3- yl]phenyl]azetidin-1-yl]-[(3S)-3- (1H-1,2,4-triazol-5- yl)pyrrolidin-1-yl]methanone | 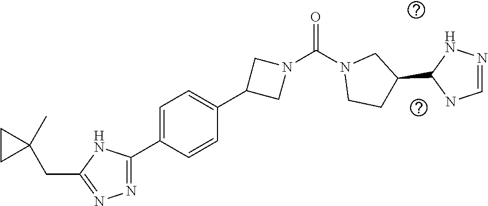 [SMILES: CC1(Cc2nnc(-c3ccc(C4CN(C(=O)N5CC[C@H](C6NC=NN6)C5)C4)cc3)[nH]2)CC1] | 433.4 [M + H]⁺ |
| 753 | [3-[4-(4-Chloro-2- methylsulfonyl- phenyl)phenyl]azetidin-1-yl]-[3- (5-cyclopropyl-1H-1,2,4-triazol- 3-yl)pyrrolidin-1-yl]methanone | 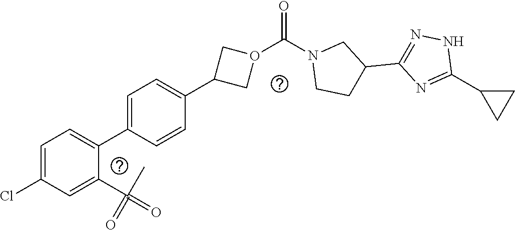 | 526.4 [M + H]⁺ |
| 755 | [4-[(2-Chloro-4-methylsulfonyl- phenyl)methoxy]-1-piperidyl]- [(3S)-3-(1H-1,2,4-triazol-5- yl)pyrrolidin-1-yl]methanone | 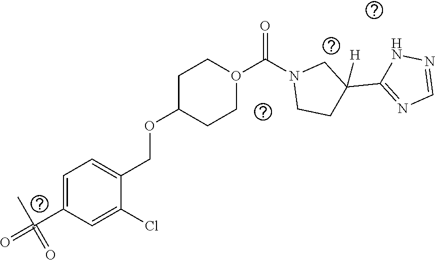 | 468.2 [M + H]⁺ |
| 756 | (3S)-1-[4-[(2-Chloro-4- methylsulfonyl- phenyl)methoxy]piperidine-1- carbonyl]pyrrolidine-3- carboxamide | 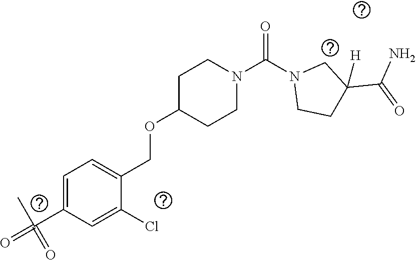 | 444.2 [M + H]⁺ |
| 757 | [4-[[4-Methylsulfonyl-3- (trifluoromethyl)phenoxy] methyl]-1-piperidyl]- [(3S)-3-(1H- 1,2,4-triazol-5-yl)pyrrolidin-1- yl]methanone | 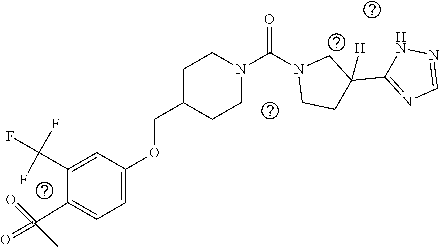 | 502.2 [M + H]⁺ |
| 758 | (3S)-1-[[4-[4-Methylsulfonyl-3- (trifluoromethyl)phenoxy] methyl]piperidine-1- carbonyl]pyrrolidine-3- carboxamide | 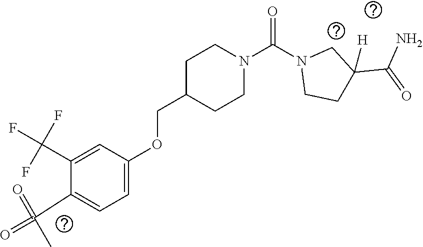 | 478.2 [M + H]⁺ |
| 759 | [3-[3-(5-Cyclopropyl-3-methyl- pyrazol-1-yl)-1- bicyclo[1.1.1]pentanyl]azetidin- 1-yl]-[(3S)-3-(1H-1,2,4-triazol- 5-yl)pyrrolidin-1-yl]methanone | 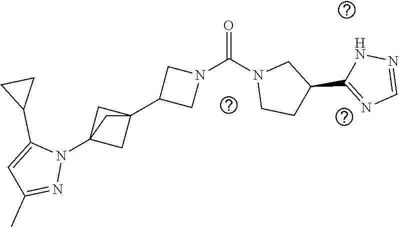 [SMILES: Cc1cc(C2CC2)n(C23CC(C4CN(C(=O)N5CC[C@H](c6ncn[nH]6)C5)C4)(C2)C3)n1] | 408.3 [M + H]⁺ |
| 760 | [3-[3-(3,5-Dimethylpyrazol-1- yl)-1- bicyclo[1.1.1]pentanyl]azetidin- 1-yl]-[(3S)-3-(1H-1,2,4-triazol- 5-yl)pyrrolidin-1-yl]methanone | 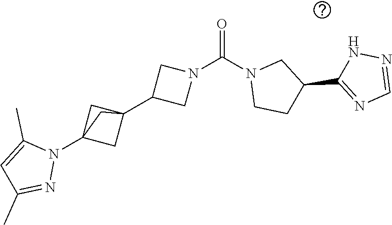 [SMILES: Cc1cc(C)n(C23CC(C4CN(C(=O)N5CC[C@H](c6ncn[nH]6)C5)C4)(C2)C3)n1] | 382.3 [M + H]⁺ |
| 762 | [4-[[2-Fluoro-4- (trifluoromethyl) phenyl]methoxy]- 1-piperidyl]-[(3S)-3-(1H-1,2,4- triazol-5-yl)pyrrolidin-1- yl]methanone | 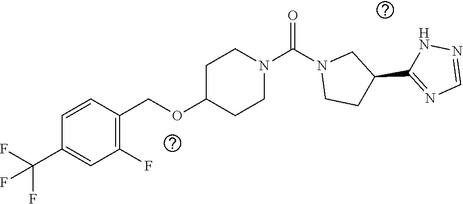 [SMILES: O=C(N1CCC(OCc2ccc(C(F)(F)F)cc2F)CC1)N1CC[C@H](c2ncn[nH]2)C1] | 442.2 [M + H]⁺ |
| 763 | (3S)-1-[4-[[2-Fluoro-4- (trifluoromethoxy)phenyl] methoxy]piperidine-1- carbonyl]pyrrolidine-3- carboxamide | 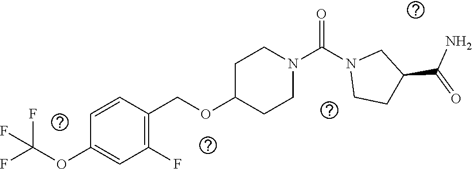 [SMILES: NC(=O)[C@H]1CCN(C(=O)N2CCC(OCc3ccc(OC(F)(F)F)cc3F)CC2)C1] | 434.2 [M + H]⁺ |
| 764 | (3S)-1-[4-[[2-Fluoro-4- (trifluoromethyl)phenyl] methoxy]piperidine-1- carbonyl]pyrrolidine-3- carboxamide | 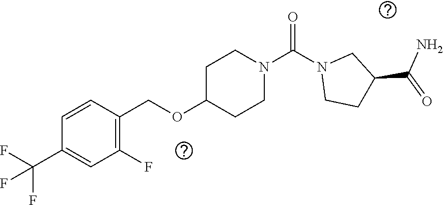 [SMILES: NC(=O)[C@H]1CCN(C(=O)N2CCC(OCc3ccc(C(F)(F)F)cc3F)CC2)C1] | 418.2 [M + H]⁺ |
| 766 | [4-[[2-Methylsulfonyl-4- (trifluoromethyl) phenoxy]methyl]- 1-piperidyl]-[(3S)-3-(1H-1,2,4- triazol-5-yl)pyrrolidin-1- yl]methanone | 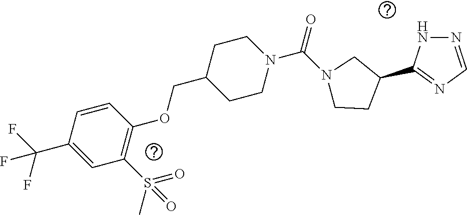 [SMILES: CS(=O)(=O)c1cc(C(F)(F)F)ccc1OCC1CCN(C(=O)N2CC[C@H](c3ncn[nH]3)C2)CC1] | 502.2 [M + H]⁺ |
| 767 | [3-[4-(4-Chloro-2- methylsulfonyl- phenyl)phenyl]azetidin-1-yl]-[4- (5-cyclopropyl-1H-1,2,4-triazol- 3-yl)piperazin-1-yl]methanone | 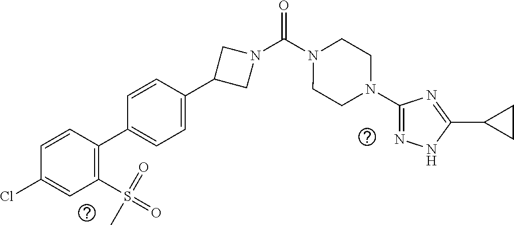 [SMILES: CS(=O)(=O)c1cc(Cl)ccc1-c1ccc(C2CN(C(=O)N3CCN(c4n[nH]c(C5CC5)n4)CC3)C2)cc1] | 541.2 [M + H]⁺ |
| 769 | [4-(5-Chlorooxazolo[4,5- b]pyridin-2-yl)piperazin-1-yl]- [3-[[2-fluoro-4- (trifluoromethoxy)phenyl] methoxy]azetidin-1- yl]methanone | 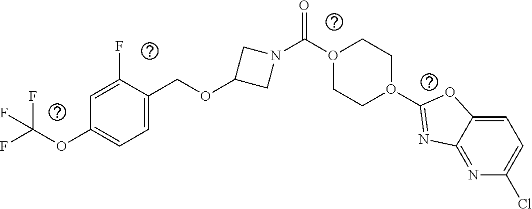 | 530.2 [M + H]⁺ |
| 770 | 4-[[2-[(3S)-3-(1H-1,2,4-Triazol- 5-yl)pyrrolidine-1-carbonyl]-2- azaspiro[3.3]heptan-6- yl]methyl]benzenesulfonamide | 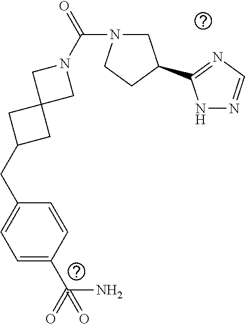 | 431.3 [M + H]⁺ |
| 773 | [(3S)-3-(4H-1,2,4-Triazol-3- yl)pyrrolidin-1-yl]-[6-[[4-[3- (trifluoromethyl)azetidin-1- yl]sulfonylphenyl]methyl]-2- azaspiro[3.3]heptan-2- yl]methanone | 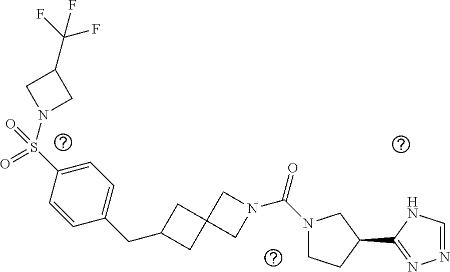 [SMILES: O=C(N1CC[C@H](c2nnc[nH]2)C1)N1CC2(CC(Cc3ccc(S(=O)(=O)N4CC(C(F)(F)F)C4)cc3)C2)C1] | 537.3 [M − H]⁻ |
| 774 | [(3S)-3-(1H-1,2,4-Triazol-5- yl)pyrrolidin-1-yl]-[(6S)-6-[[4- (trifluoromethylsulfonyl)phenyl] methyl]-2-azaspiro[3.4]octan-2- yl]methanone | 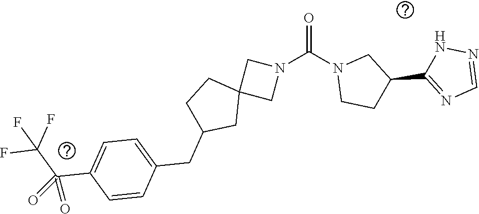 | 498.3 [M + H]⁺ |
| 775 | [(3S)-3-(1H-1,2,4-Triazol-5- yl)pyrrolidin-1-yl]-[(6R)-6-[[4- (trifluoromethylsulfonyl)phenyl] methyl]-2-azaspiro[3.4]octan-2- yl]methanone | 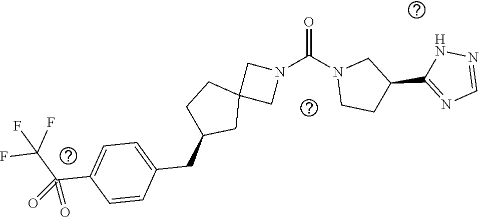 | 498.3 [M + H]⁺ |
| 777 | [(3S)-3-(1H-1,2,4-Triazol-5- yl)pyrrolidin-1-yl]-[(6R)-6-[[3- (trifluoromethylsulfonyl)phenyl] methyl]-2-azaspiro[3.4]octan-2- yl]methanone | 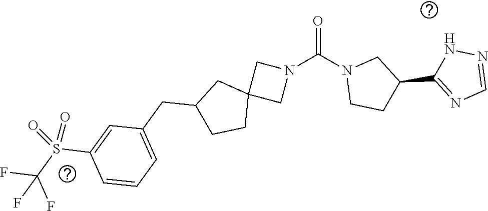 [SMILES: O=C(N1CC[C@H](c2ncn[nH]2)C1)N1CC2(CCC(Cc3cccc(S(=O)(=O)C(F)(F)F)c3)C2)C1] | 498.3 [M + H]⁺ |
| 778 | [6-[[1-Methyl-3- (trifluoromethyl)pyrazol-4- yl]methyl]-2- azaspiro[3.3]heptan-2-yl]-[(3S)- 3-(1H-1,2,4-triazol-5- yl)pyrrolidin-1-yl]methanone | 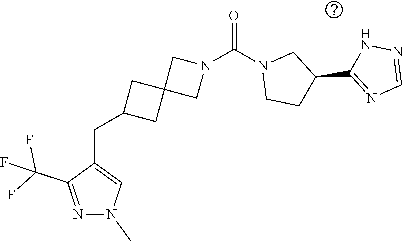 [SMILES: Cn1cc(CC2CC3(C2)CN(C(=O)N2CC[C@H](c4ncn[nH]4)C2)C3)c(C(F)(F)F)n1] | 424.3 [M + H]⁺ |
| 779 | [6-[[2-Methyl-5- (trifluoromethyl)pyrazol-3- yl]methyl]-2- azaspiro[3.3]heptan-2-yl]-[(3S)- 3-(1H-1,2,4-triazol-5- yl)pyrrolidin-1-yl]methanone | 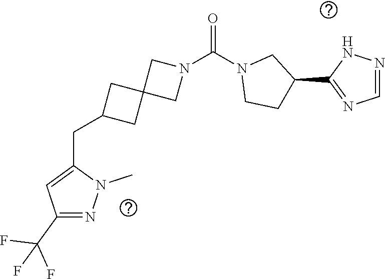 [SMILES: Cn1nc(C(F)(F)F)cc1CC1CC2(C1)CN(C(=O)N1CC[C@H](c3ncn[nH]3)C1)C2] | 424.3 [M + H]⁺ |
| 781 | [6-[[2-Methyl-4- (trifluoromethyl)pyrazol-3- yl]methyl]-2- azaspiro[3.3]heptan-2-yl]-[(3S)- 3-(1H-1,2,4-triazol-5- yl)pyrrolidin-1-yl]methanone | 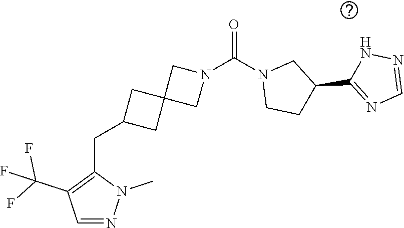 [SMILES: Cn1ncc(C(F)(F)F)c1CC1CC2(C1)CN(C(=O)N1CC[C@H](c3ncn[nH]3)C1)C2] | 424.3 [M + H]⁺ |
| 782 | [(3S)-3-(1H-1,2,4-Triazol-5- yl)pyrrolidin-1-yl]-[6-[[1-(2,2,2- trifluoroethyl)pyrazol-4- yl]methyl]-2- azaspiro[3.3]heptan-2- yl]methanone | 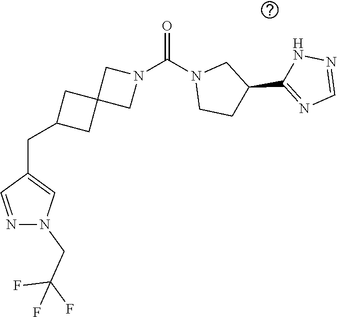 [SMILES: O=C(N1CC[C@H](c2ncn[nH]2)C1)N1CC2(CC(Cc3cnn(CC(F)(F)F)c3)C2)C1] | 424.3 [M + H]⁺ |
| 783 | 4-[[2-[(3S)-3-(1H-1,2,4-Triazol- 5-yl)pyrrolidine-1-carbonyl]-2- azaspiro[3.3]heptan-6- yl]methyl]-2- (trifluoromethoxy)benzamide | 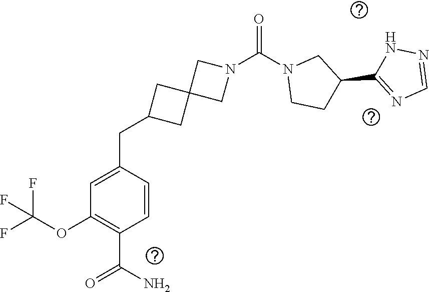 [SMILES: NC(=O)c1ccc(CC2CC3(C2)CN(C(=O)N2CC[C@H](c4ncn[nH]4)C2)C3)cc1OC(F)(F)F] | 479.2 [M + H]⁺ |
| 784 | [(3S)-3-(4H-1,2,4-Triazol-3- yl)pyrrolidin-1-yl]-[3-[5-[1- (trifluoromethyl)cyclopropyl]-2- pyridyl]azetidin-1-yl]methanone | 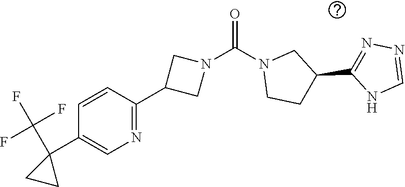 [SMILES: O=C(N1CC(c2ccc(C3(C(F)(F)F)CC3)cn2)C1)N1CC[C@H](c2nnc[nH]2)C1] | 407.1 [M + H]⁺ |
| 785 | [(3S)-3-(4H-1,2,4-Triazol-3- yl)pyrrolidin-1-yl]-[3-[[6- (trifluoromethyl)-3- pyridyl]methoxymethyl]azetidin- 1-yl]methanone | 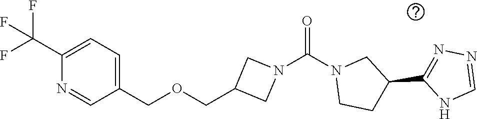 [SMILES: O=C(N1CC(COCc2ccc(C(F)(F)F)nc2)C1)N1CC[C@H](c2nnc[nH]2)C1] | 411.1 [M + H]⁺ |
| 786 | [3-[4-(1-Methyl- sulfonylcyclopropyl)phenyl] azetidin-1-yl]-[(3S)-3-(1H- 1,2,4-triazol-5-yl)pyrrolidin-1- yl]methanone | 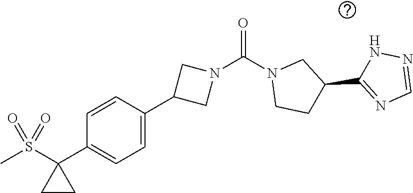 [SMILES: CS(=O)(=O)C1(c2ccc(C3CN(C(=O)N4CC[C@H](c5ncn[nH]5)C4)C3)cc2)CC1] | 416.2 [M + H]⁺ |
| 787 | [4-[(3-Chloro-5-methylsulfonyl- phenoxy)methyl]-1-piperidyl]- [(3S)-3-(1H-1,2,4-triazol-5- yl)pyrrolidin-1-yl]methanone | 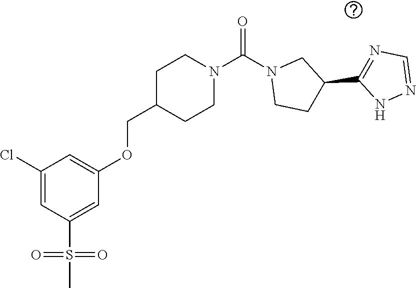 [SMILES: CS(=O)(=O)c1cc(Cl)cc(OCC2CCN(C(=O)N3CC[C@H](c4ncn[nH]4)C3)CC2)c1] | 468.2 [M + H]⁺ |
| 788 | 5-[[2-[(3S)-3-(1H-1,2,4-Triazol- 5-yl)pyrrolidine-1-carbonyl]-2- azaspiro[3.3]heptan-6- yl]methyl]-2- (trifluoromethoxy)benzamide | 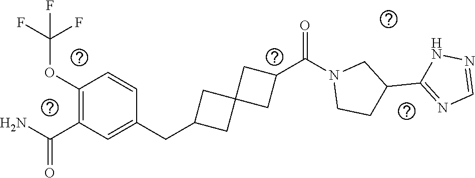 [SMILES: NC(=O)c1cc(CC2CC3(C2)CC(C(=O)N2CCC(c4ncn[nH]4)C2)C3)ccc1OC(F)(F)F] | 479.2 [M + H]⁺ |
| 789 | [rac-(3S)-3-(1H-1,2,4-Triazol-5- yl)pyrrolidin-1-yl]-[3-[4- (trifluoromethylsulfonimidoyl) phenyl]azetidin-1-yl]methanone | 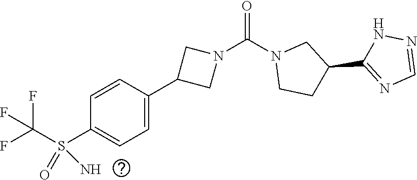 | 429.2 [M + H]⁺ |
| 791 | Methyl 4-[[2-[(3S)-3-(1H-1,2,4- triazol-5-yl)pyrrolidine-1- carbonyl]-2-azaspiro[3.3]heptan- 6-yl]methyl]-2- (trifluoromethoxy)benzoate | 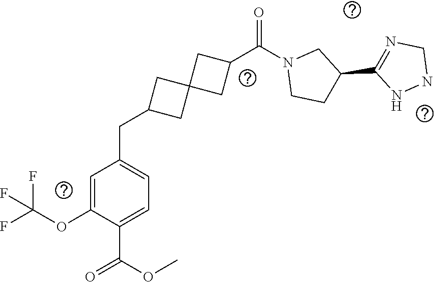 [SMILES: COC(=O)c1ccc(CC2CC3(C2)CC(C(=O)N2CC[C@H](C4=NCNN4)C2)C3)cc1OC(F)(F)F] | 492.2 [M − H]⁻ |
| 792 | [4-[[2-Methylsulfonyl-4- (trifluoromethoxy)phenoxy] methyl]-1-piperidyl]-[(3S)-3- (4H-1,2,4-triazol-3-yl)pyrrolidin- 1-yl]methanone | 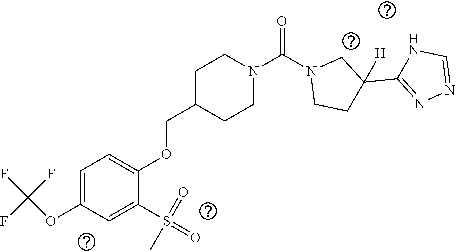 [SMILES: CS(=O)(=O)c1cc(OC(F)(F)F)ccc1OCC1CCN(C(=O)N2CCC(c3nnc[nH]3)C2)CC1] | 518.2 [M − H]⁻ |
| 793 | [(3S)-3-(4H-1,2,4-Triazol-3- yl)pyrrolidin-1-yl]-[4-[[6- (trifluoromethyl)-3- pyridyl]methoxymethyl]-1- piperidyl]methanone | 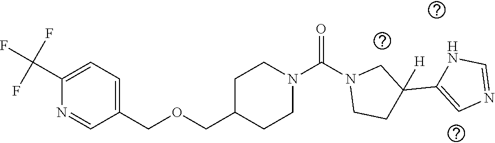 [SMILES: O=C(N1CCC(COCc2ccc(C(F)(F)F)nc2)CC1)N1CCC(c2cnc[nH]2)C1] | 439.2 [M − H]⁻ |
| 794 | [(3S)-3-(4H-1,2,4-Triazol-3- yl)pyrrolidin-1-yl]-[4-[[6- (trifluoromethyl)-3- pyridyl]oxymethyl]-1- piperidyl]methanone | 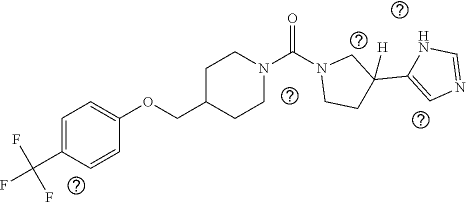 [SMILES: O=C(N1CCC(COc2ccc(C(F)(F)F)cc2)CC1)N1CCC(c2cnc[nH]2)C1] | 425.2 [M + H]⁺ |
| 795 | [(3S)-3-(4H-1,2,4-Triazol-3- yl)pyrrolidin-1-yl]-[4-[[6- (trifluoromethyl)-3- pyridyl]methoxy]-1- piperidyl]methanone | 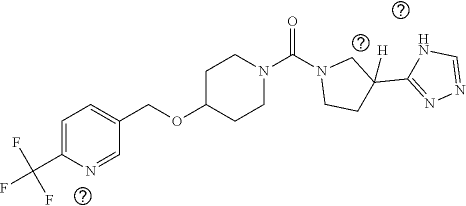 [SMILES: O=C(N1CCC(OCc2ccc(C(F)(F)F)nc2)CC1)N1CCC(c2nnc[nH]2)C1] | 425.2 [M + H]⁺ |
| 796 | [4-[[4-Fluoro-3-(trifluoro- methyl)phenoxy]methyl]- 1-piperidyl]-[(3S)-3-(4H-1,2,4- triazol-3-yl)pyrrolidin-1- yl]methanone | 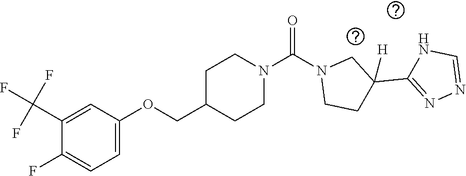 [SMILES: O=C(N1CCC(COc2ccc(F)c(C(F)(F)F)c2)CC1)N1CCC(c2nnc[nH]2)C1] | 442.2 [M + H]⁺ |
| 797 | [4-[[3-Fluoro-5-(trifluoro- methyl)phenoxy]methyl]- 1-piperidyl]-[(3S)-3-(4H-1,2,4- triazol-3-yl)pyrrolidin-1- yl]methanone | 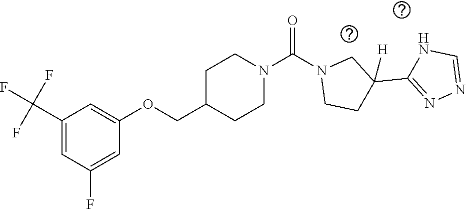 [SMILES: O=C(N1CCC(COc2cc(F)cc(C(F)(F)F)c2)CC1)N1CCC(c2nnc[nH]2)C1] | 442.2 [M + H]⁺ |
| 798 | [3-(3-Isobutyl-1H-pyrazol-5- yl)pyrrolidin-1-yl]-[6-[[5- (trifluoromethyl)pyrazin-2- yl]methyl]-2- azaspiro[3.3]heptan-2- yl]methanone | 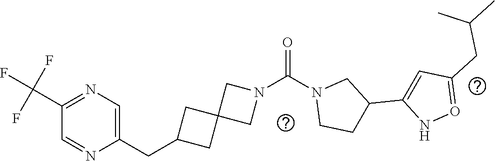 | 477.3 [M + H]⁺ |
| 799 | [(3S)-3-(4H-1,2,4-Triazol-3- yl)pyrrolidin-1-yl]-[6-[[5- (trifluoromethylsulfonyl)-3- pyridyl]methyl]-2- azaspiro[3.3]heptan-2- yl]methanone | 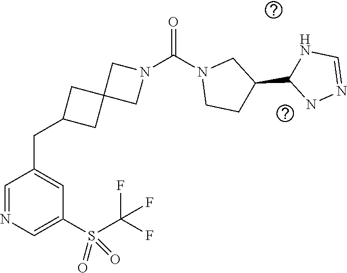 [SMILES: O=C(N1CC[C@H](C2NC=NN2)C1)N1CC2(CC(Cc3cncc(S(=O)(=O)C(F)(F)F)c3)C2)C1] | 485.2 [M + H]⁺ |
| 800 | [6-[[3-Fluoro-5- (trifluoromethylsulfonyl)phenyl] methyl]-2-azaspiro[3.3]heptan- 2-yl]-[(3S)-3-(4H-1,2,4-triazol- 3-yl)pyrrolidin-1-yl]methanone | 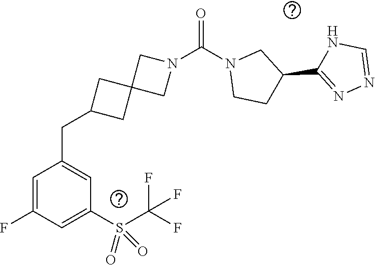 [SMILES: O=C(N1CC[C@H](c2nnc[nH]2)C1)N1CC2(CC(Cc3cc(F)cc(S(=O)(=O)C(F)(F)F)c3)C2)C1] | 502.2 [M + H]⁺ |
| 801 | N′-[2-[3-[1-[(3S)-3-(4H-1,2,4- Triazol-3-yl)pyrrolidine-1- carbonyl]azetidin-3-yl]-1- bicyclo[1.1.1]pentanyl]acetyl] cyclopropanecarbohydrazide | 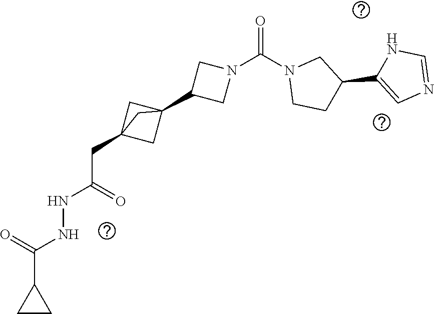 [SMILES: O=C(C[C@]12C[C@](C3CN(C(=O)N4CC[C@H](c5cnc[nH]5)C4)C3)(C1)C2)NNC(=O)C1CC1] | 428.3 [M + H]⁺ |
| 802 | [4-[[2-Methylsulfonyl-4- (trifluoromethoxy)phenyl] methoxy]-1-piperidyl]-[(3S)-3- (1H-1,2,4-triazol-5-yl)pyrrolidin- 1-yl]methanone | 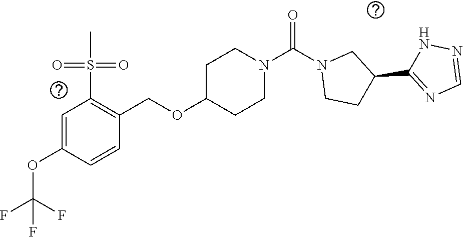 [SMILES: CS(=O)(=O)c1cc(OC(F)(F)F)ccc1COC1CCN(C(=O)N2CC[C@H](c3ncn[nH]3)C2)CC1] | 518.2 [M + H]⁺ |
| 803 | (3S)-1-[4-[[2-Methylsulfonyl-4- (trifluoromethoxy)phenyl] methoxy]piperidine-1- carbonyl]pyrrolidine-3- carboxamide | 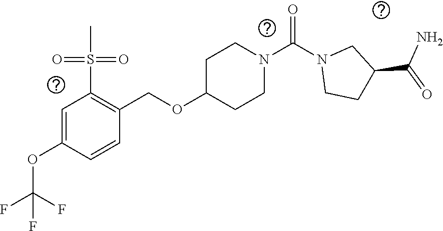 [SMILES: CS(=O)(=O)c1cc(OC(F)(F)F)ccc1COC1CCN(C(=O)N2CC[C@H](C(N)=O)C2)CC1] | 494.1 [M + H]⁺ |
| 805 | 2-Methoxy-4-[[1-[(3S)-3-(1H- 1,2,4-triazol-5-yl)pyrrolidine-1- carbonyl]-4- piperidyl]oxymethyl]benzonitrile | 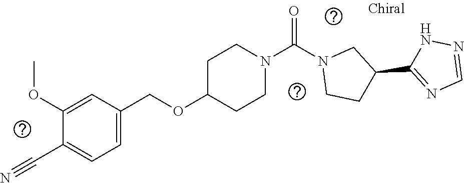 [SMILES: COc1cc(COC2CCN(C(=O)N3CC[C@H](c4ncn[nH]4)C3)CC2)ccc1C#N] | 411.2 [M + H]⁺ |
| 806 | (3S)-1-[2-(4-Chloro-2- methylsulfonyl-phenyl)-6- azaspiro[3.4]octane-6- carbonyl]pyrrolidine-3- carboxamide | 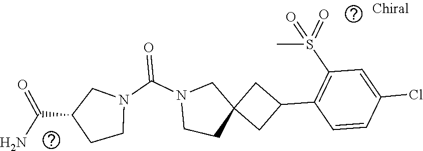 | 440.1 [M + H]⁺ |
| 807 | 2-Methoxy-4-[[1-[(3S)-3-(1H- 1,2,4-triazol-5-yl)pyrrolidine-1- carbonyl]-4- piperidyl]oxymethyl]benzamide | 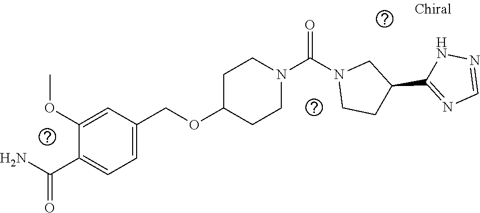 [SMILES: COc1cc(COC2CCN(C(=O)N3CC[C@H](c4ncn[nH]4)C3)CC2)ccc1C(N)=O] | 429.2 [M + H]⁺ |
| 809 | (3S)-1-[2-[(4-Chloro-2- methylsulfonyl-phenyl)methyl]- 6-azaspiro[3.4]octane-6- carbonyl]pyrrolidine-3- carboxamide | 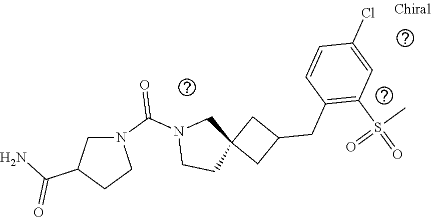 | 454.1 [M + H]⁺ |
| 810 | [(3S)-3-(1H-1,2,4-Triazol-5- yl)pyrrolidin-1-yl]-[6-[3- (trifluoromethoxy)phenyl] sulfonyl-2-azaspiro[3.3] heptan-2-yl]methanone | 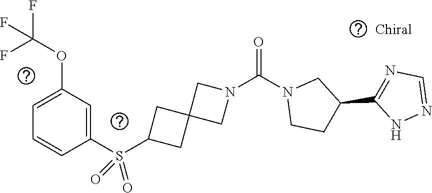 [SMILES: O=C(N1CC[C@H](c2ncn[nH]2)C1)N1CC2(CC(S(=O)(=O)c3cccc(OC(F)(F)F)c3)C2)C1] | 486.2 [M + H]⁺ |
| 811 | [(3S)-3-(1H-1,2,4-Triazol-5- yl)pyrrolidin-1-yl]-[7-[3- (trifluoromethyl)phenyl]sulfonyl- 2-azaspiro[3.5]nonan-2- yl]methanone | 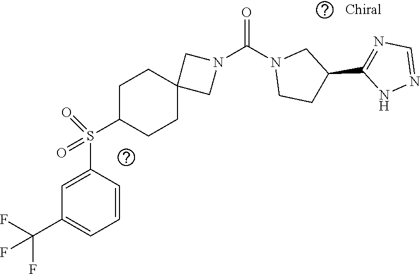 [SMILES: O=C(N1CC[C@H](c2ncn[nH]2)C1)N1CC2(CCC(S(=O)(=O)c3cccc(C(F)(F)F)c3)CC2)C1] | 498.2 [M + H]⁺ |
| 812 | [(3S)-3-(1H-1,2,4-Triazol-5- yl)pyrrolidin-1-yl]-[7-[4- (trifluoromethyl)phenyl]sulfonyl- 2-azaspiro[3.5]nonan-2- yl]methanone | 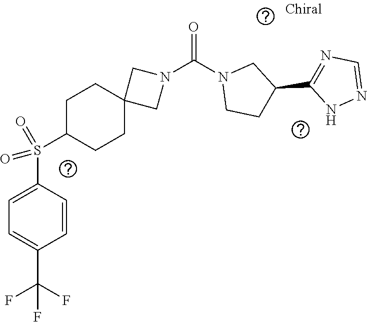 [SMILES: O=C(N1CC[C@H](c2ncn[nH]2)C1)N1CC2(CCC(S(=O)(=O)c3ccc(C(F)(F)F)cc3)CC2)C1] | 498.2 [M + H]⁺ |
| 814 | [3-[3-(4-Methylsulfonylphenyl)- 1- bicyclo[1.1.1]pentanyl]azetidin- 1-yl]-[(3S)-3-(1H-1,2,4-triazol- 5-yl)pyrrolidin-1-yl]methanone | 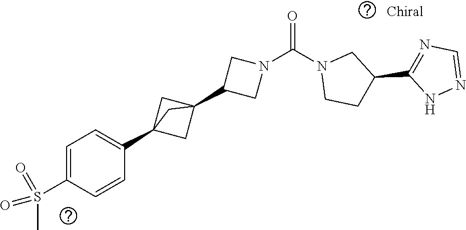 [SMILES: CS(=O)(=O)c1ccc([C@]23C[C@](C4CN(C(=O)N5CC[C@H](c6ncn[nH]6)C5)C4)(C2)C3)cc1] | 442.3 [M + H]⁺ |
| 815 | [(3S)-3-(1H-1,2,4-Triazol-5- yl)pyrrolidin-1-yl]-[6-[[4- (trifluoromethylsulfonimidoyl) phenyl]methyl]-2- azaspiro[3.3]heptan-2- yl]methanone | 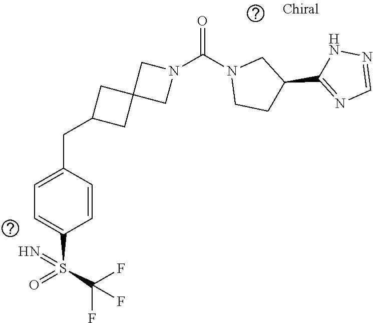 [SMILES: N=[S@](=O)(c1ccc(CC2CC3(C2)CN(C(=O)N2CC[C@H](c4ncn[nH]4)C2)C3)cc1)C(F)(F)F] | 483.2 [M + H]⁺ |
| 816 | [(3S)-3-(1H-1,2,4-Triazol-5- yl)pyrrolidin-1-yl]-[6-[[4- (trifluoromethylsulfonimidoyl) phenyl]methyl]-2- azaspiro[3.3]heptan-2- yl]methanone | 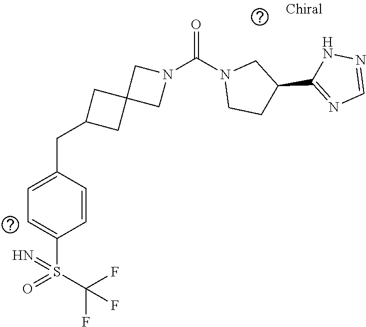 [SMILES: N=S(=O)(c1ccc(CC2CC3(C2)CN(C(=O)N2CC[C@H](c4ncn[nH]4)C2)C3)cc1)C(F)(F)F] | 483.2 [M + H]⁺ |
| 817 | [(3S)-3-(1H-1,2,4-Triazol-5- yl)pyrrolidin-1-yl]-[6-[[3- (trifluoromethylsulfonimidoyl) phenyl]methyl]-2- azaspiro[3.3]heptan-2- yl]methanone | 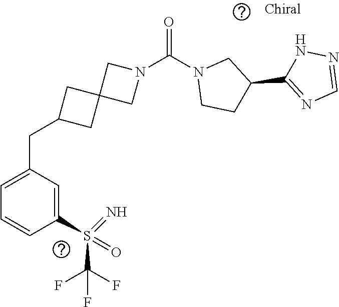 [SMILES: N=[S@@](=O)(c1cccc(CC2CC3(C2)CN(C(=O)N2CC[C@H](c4ncn[nH]4)C2)C3)c1)C(F)(F)F] | 483.2 [M + H]⁺ |
| 818 | [(3S)-3-(1H-1,2,4-Triazol-5- yl)pyrrolidin-1-yl]-[6-[[3- (trifluoromethylsulfonimidoyl) phenyl]methyl]-2- azaspiro[3.3]heptan-2- yl]methanone | 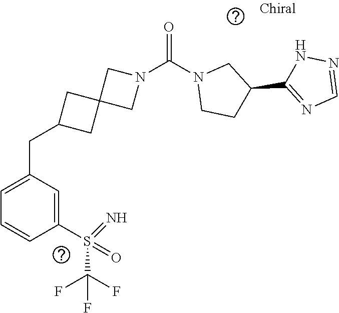 [SMILES: N=[S@@](=O)(c1cccc(CC2CC3(C2)CN(C(=O)N2CC[C@H](c4ncn[nH]4)C2)C3)c1)C(F)(F)F] | 483.2 [M + H]⁺ |
| 819 | [6-[[3-(Methylsulfonimidoyl)-5- (trifluoromethyl)phenyl]methyl]- 2-azaspiro[3.3]heptan-2-yl]-[rac- (3S)-3-(1H-1,2,4-triazol-5- yl)pyrrolidin-1-yl]methanone | 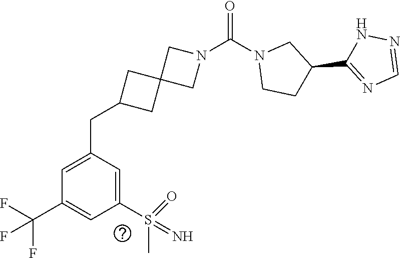 [SMILES: CS(=N)(=O)c1cc(CC2CC3(C2)CN(C(=O)N2CC[C@H](c4ncn[nH]4)C2)C3)cc(C(F)(F)F)c1] | 497.2 [M + H]⁺ |
| 820 | [3-[[2-Fluoro-4- (trifluoromethylsulfonimidoyl) phenyl]methoxy]azetidin-1-yl]- [rac-(3S)-3-(1H-1,2,4-triazol-5- yl)pyrrolidin-1-yl]methanone | 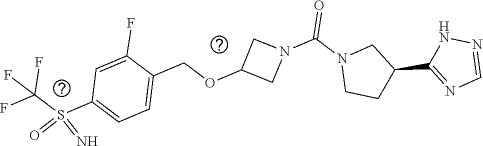 [SMILES: N=S(=O)(c1ccc(COC2CN(C(=O)N3CC[C@H](c4ncn[nH]4)C3)C2)c(F)c1)C(F)(F)F] | 477.1 [M + H]⁺ |
| 823 | [rac-(3S)-3-(1H-1,2,4-Triazol-5- yl)pyrrolidin-1-yl]-[6-[[3- (trifluoromethoxy)phenyl] sulfonimidoyl]-2-azaspiro[3.3] heptan-2-yl]methanone | 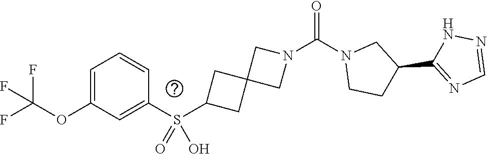 | 485.2 [M + H]⁺ |
| 824 | [6-[[3-Fluoro-5- (trifluoromethylsulfonimidoyl) phenyl]methyl]-2- azaspiro[3.3]heptan-2-yl]-[rac- (3S)-3-(1H-1,2,4-triazol-5- yl)pyrrolidin-1-yl]methanone | 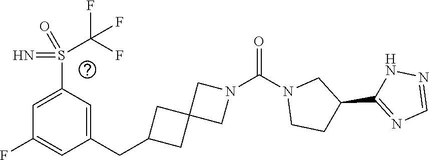 [SMILES: N=S(=O)(c1cc(F)cc(CC2CC3(C2)CN(C(=O)N2CC[C@H](c4ncn[nH]4)C2)C3)c1)C(F)(F)F] | 501.2 [M + H]⁺ |
| 825 | [rac-(3S)-3-(1H-1,2,4-Triazol-5- yl)pyrrolidin-1-yl]-[7-[[4- (trifluoromethyl)phenyl] sulfonimidoyl]-2-azaspiro[3.5] nonan-2-yl]methanone | 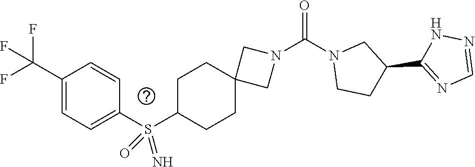 [SMILES: N=S(=O)(c1ccc(C(F)(F)F)cc1)C1CCC2(CC1)CN(C(=O)N1CC[C@H](c3ncn[nH]3)C1)C2] | 497.3 [M + H]⁺ |
| 826 | [rac-(3S)-3-(1H-1,2,4-Triazol-5- yl)pyrrolidin-1-yl]-[7-[[3- (trifluoromethyl)phenyl] sulfonimidoyl]-2-azaspiro[3.5] nonan-2-yl]methanone | 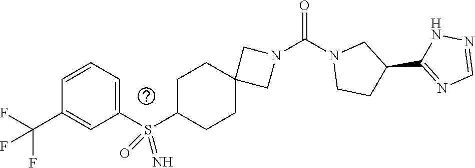 [SMILES: N=S(=O)(c1cccc(C(F)(F)F)c1)C1CCC2(CC1)CN(C(=O)N1CC[C@H](c3ncn[nH]3)C1)C2] | 497.3 [M + H]⁺ |
| 827 | [3-[4-[2- (Methylsulfonimidoyl)phenyl] phenyl]azetidin-1-yl]-[rac-(3S)- 3-(1H-1,2,4-triazol-5- yl)pyrrolidin-1-yl]methanone | 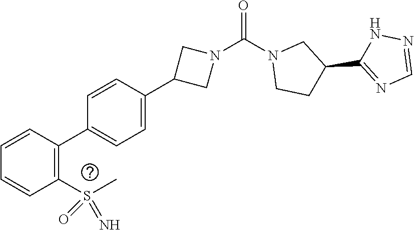 [SMILES: CS(=N)(=O)c1ccccc1-c1ccc(C2CN(C(=O)N3CC[C@H](c4ncn[nH]4)C3)C2)cc1] | 451.3 [M + H]⁺ |
| 828 | [3-[(8R)-8-Methyl-8-oxo- 8lambda6-thia-9- azatricyclo[8.4.0.02,7]tetradeca- 1(10),2(7),3,5,8,11,13-heptaen- 12-yl]azetidin-1-yl]-[(3S)-3-(1H- 1,2,4-triazol-5-yl)pyrrolidin-1- yl]methanone | 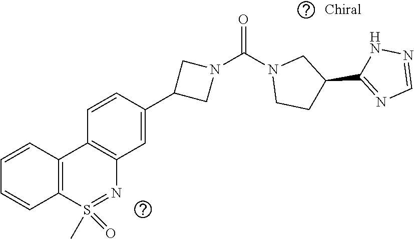 [SMILES: CS1(=O)=Nc2cc(C3CN(C(=O)N4CC[C@H](c5ncn[nH]5)C4)C3)ccc2-c2ccccc21] | 449.3 [M + H]⁺ |
| 829 | [3-[(8S)-8-Methyl-8-oxo- 8lambda6-thia-9- azatricyclo[8.4.0.02,7]tetradeca- 1(10),2(7),3,5,8,11,13-heptaen- 12-yl]azetidin-1-yl]-[(3S)-3-(1H- 1,2,4-triazol-5-yl)pyrrolidin-1- yl]methanone | 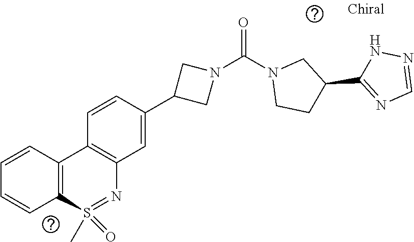 [SMILES: C[S@@]1(=O)=Nc2cc(C3CN(C(=O)N4CC[C@H](c5ncn[nH]5)C4)C3)ccc2-c2ccccc21] | 449.2 [M + H]⁺ |
| 830 | [3-[3-(4-Fluoro-2- methylsulfonyl-phenyl)-1- bicyclo[1.1.1]pentanyl]azetidin- 1-yl]-[(3S)-3-(1H-1,2,4-triazol- 5-yl)pyrrolidin-1-yl]methanone | 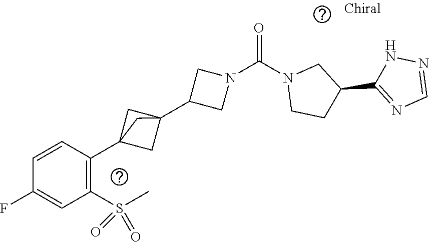 [SMILES: CS(=O)(=O)c1cc(F)ccc1C12CC(C3CN(C(=O)N4CC[C@H](c5ncn[nH]5)C4)C3)(C1)C2] | 460.2 [M + H]⁺ |
| 832 | [(3S)-3-(4H-1,2,4-Triazol-3- yl)pyrrolidin-1-yl]-[4-[[5- (trifluoromethyl)-2- pyridyl]methyl]piperazin-1- yl]methanone | 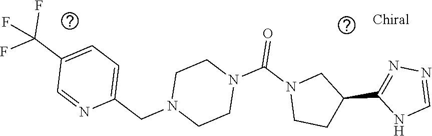 [SMILES: O=C(N1CCN(Cc2ccc(C(F)(F)F)cn2)CC1)N1CC[C@H](c2nnc[nH]2)C1] | 410.2 [M + H]⁺ |
| 833 | [4-[(2-Methyloxazol-4- yl)methyl]piperazin-1-yl]-[(3S)- 3-(1H-1,2,4-triazol-5- yl)pyrrolidin-1-yl]methanone | 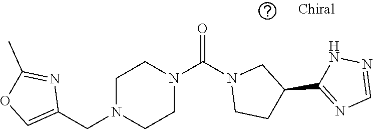 [SMILES: Cc1nc(CN2CCN(C(=O)N3CC[C@H](c4ncn[nH]4)C3)CC2)co1] | 346.1 [M + H]⁺ |
| 834 | [4-[(4-Methylthiazol-5- yl)methyl]piperazin-1-yl]-[(3S)- 3-(1H-1,2,4-triazol-5- yl)pyrrolidin-1-yl]methanone | 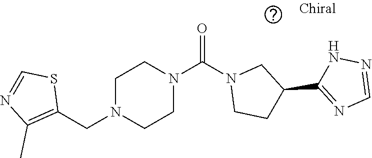 [SMILES: Cc1ncsc1CN1CCN(C(=O)N2CC[C@H](c3ncn[nH]3)C2)CC1] | 362.1 [M + H]⁺ |
| 835 | [4-[(5-Chloro-1,3-dimethyl- pyrazol-4-yl)methyl]piperazin-1- yl]-[(3S)-3-(1H-1,2,4-triazol-5- yl)pyrrolidin-1-yl]methanone | 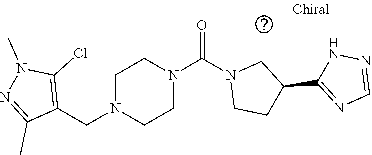 | 437.3 [M + HCOO]⁻ |
| 836 | 4-[[2-[(3S)-3-(1H-1,2,4-Triazol- 5-yl)pyrrolidine-1-carbonyl]-2- azaspiro[3.3]heptan-6-yl]oxy]-2- (trifluoromethyl)benzonitrile | 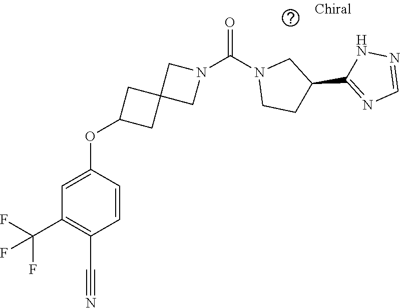 [SMILES: N#Cc1ccc(OC2CC3(C2)CN(C(=O)N2CC[C@H](c4ncn[nH]4)C2)C3)cc1C(F)(F)F] | 447.2 [M + H]⁺ |
| 837 | 5-Fluoro-2-[3-[1-[(3S)-3-(1H- 1,2,4-triazol-5-yl)pyrrolidine-1- carbonyl]azetidin-3-yl]-1- bicyclo[1.1.1]pentanyl] benzonitrile | 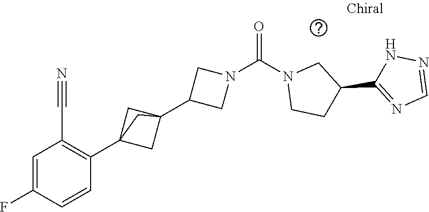 [SMILES: N#Cc1cc(F)ccc1C12CC(C3CN(C(=O)N4CC[C@H](c5ncn[nH]5)C4)C3)(C1)C2] | 407.2 [M + H]⁺ |
| 838 | [3-[3-(3-Methyl-1H-pyrazol-5- yl)-1- bicyclo[1.1.1]pentanyl]azetidin- 1-yl]-[(3S)-3-(1H-1,2,4-triazol- 5-yl)pyrrolidin-1-yl]methanone | 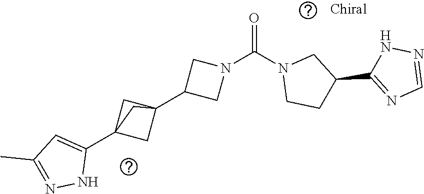 [SMILES: Cc1cc(C23CC(C4CN(C(=O)N5CC[C@H](c6ncn[nH]6)C5)C4)(C2)C3)[nH]n1] | 368.2 [M + H]⁺ |
| 839 | [6-[[1-Methyl-4- (trifluoromethyl)pyrazol-3- yl]methyl]-2- azaspiro[3.3]heptan-2-yl]-[(3S)- 3-(4H-1,2,4-triazol-3- yl)pyrrolidin-1-yl]methanone | 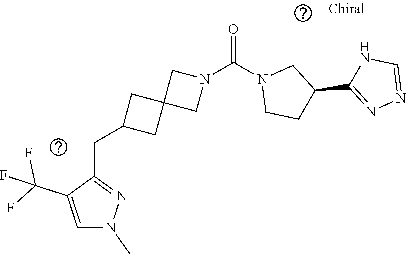 [SMILES: Cn1cc(C(F)(F)F)c(CC2CC3(C2)CN(C(=O)N2CC[C@H](c4nnc[nH]4)C2)C3)n1] | 424.2 [M + H]⁺ |
| 842 | [6-[(5-Chloro-1,3-dimethyl- pyrazol-4-yl)methyl]-2,6- diazaspiro[3.3]heptan-2-yl]- [(3S)-3-(1H-1,2,4-triazol-5- yl)pyrrolidin-1-yl]methanone | 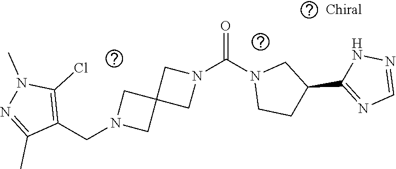 | 449.2 [M + HCOO]⁻ |
| 843 | 5-[[2-[(3S)-3-(1H-1,2,4-Triazol- 5-yl)pyrrolidine-1-carbonyl]-2- azaspiro[3.3]heptan-6-yl]oxy]-2- (trifluoromethyl)benzonitrile | 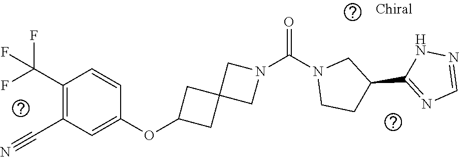 [SMILES: N#Cc1cc(OC2CC3(C2)CN(C(=O)N2CC[C@H](c4ncn[nH]4)C2)C3)ccc1C(F)(F)F] | 447.2 [M + H]⁺ |
| 844 | [6-[(5-Fluoro-2-pyridyl)oxy]-2- azaspiro[3.3]heptan-2-yl]-[(3S)- 3-(1H-1,2,4-triazol-5- yl)pyrrolidin-1-yl]methanone | 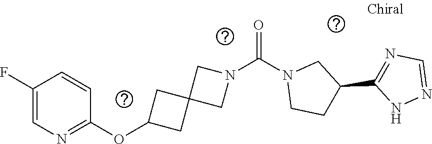 [SMILES: O=C(N1CC[C@H](c2ncn[nH]2)C1)N1CC2(CC(Oc3ccc(F)cn3)C2)C1] | 373.2 [M + H]⁺ |
| 845 | [6-[1-Methyl-3- (trifluoromethyl)pyrazol-4- yl]oxy-2-azaspiro[3.3]heptan-2- yl]-[(3S)-3-(1H-1,2,4-triazol-5- yl)pyrrolidin-1-yl]methanone | 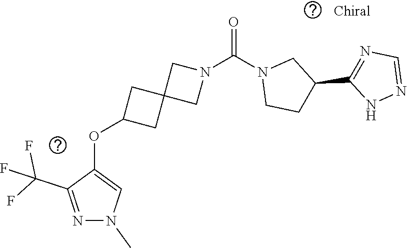 [SMILES: Cn1cc(OC2CC3(C2)CN(C(=O)N2CC[C@H](c4ncn[nH]4)C2)C3)c(C(F)(F)F)n1] | 426.2 [M + H]⁺ |
| 846 | [(3S)-3-(1H-1,2,4-Triazol-5- yl)pyrrolidin-1-yl]-[3-[[5- (trifluoromethyl)pyrazin-2- yl]amino]-1-bicyclo [1.1.1]pentanyl]methanone | 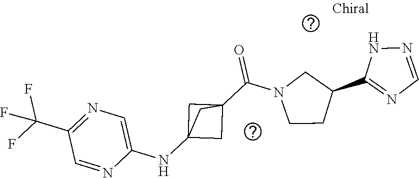 [SMILES: O=C(N1CC[C@H](c2ncn[nH]2)C1)C12CC(Nc3cnc(C(F)(F)F)cn3)(C1)C2] | 394.2 [M + H]⁺ |
| 847 | (3S)-3-(1H-1,2,4-Triazol-5- | | 485.4 |
| | yl)pyrrolidin-1-yl]-[6-[4- | | [M + H]⁺ |
| | (trifluoromethylsulfonimidoyl) | | |
| | phenoxy]-2-azaspiro[3.3]heptan- | | |
| | 2-yl]methanone | | |
| 848 | [(3S)-3-(1H-Triazol-5- | | 483.4 |
| | yl)pyrrolidin-1-yl]-[6-[[3- | | [M + H]⁺ |
| | (trifluoromethylsulfonimidoyl) | | |
| | phenyl]methyl]-2- | | |
| | azaspiro[3.3]heptan-2- | | |
| | yl]methanone | | |
| 849 | [(3S)-3-(1H-Triazol-5- | | 483.4 |
| | yl)pyrrolidin-1-yl]-[6-[[3- | | [M + H]⁺ |
| | (trifluoromethylsulfonimidoyl) | | |
| | phenyl]methyl]-2- | | |
| | azaspiro[3.3]heptan-2- | | |
| | yl]methanone | | |
| 850 | [(3S)-3-(1H-Triazol-5- | | 484.4 |
| | yl)pyrrolidin-1-yl]-[6-[[3- | | [M + H]⁺ |
| | (trifluoromethylsulfonyl)phenyl] | | |
| | methyl]-2-azaspiro[3.3]heptan- | | |
| | 2-yl]methanone | | |
| 851 | 5-[[2-[(3S)-3-(1H-Triazol-5- yl)pyrrolidine-1-carbonyl]-2- azaspiro[3.3]heptan-6- yl]methyl]-2- (trifluoromethyl)benzonitrile | 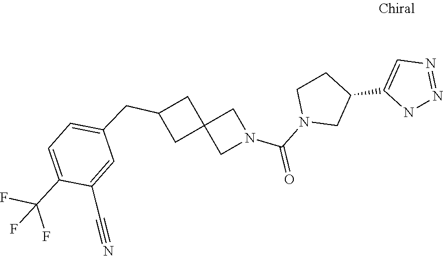 | 445.4 [M + H]⁺ |
| 852 | (3S)-1-[6-[[1-Methyl-5- (trifluoromethyl)pyrazol-3- yl]methyl]-2- azaspiro[3.3]heptane-2- carbonyl]pyrrolidine-3- carboxamide | 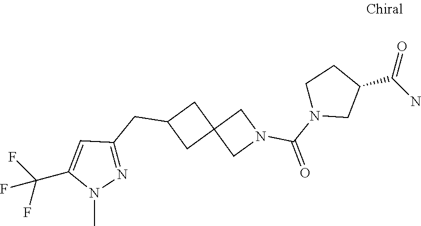 | 400.4 [M + H]⁺ |
| 853 | (3S)-1-[6-[[1-Methyl-5- (trifluoromethyl)pyrazol-4- yl]methyl]-2- azaspiro[3.3]heptane-2- carbonyl]pyrrolidine-3- carboxamide | 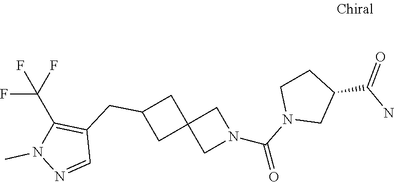 | 400.3 [M + H]⁺ |
| 854 | (3S)-1-[6-[[3-(Trifluoro- methylsulfonyl)phenyl] methyl]-2-azaspiro[3.3]heptane- 2-carbonyl]pyrrolidine-3- carboxamide | 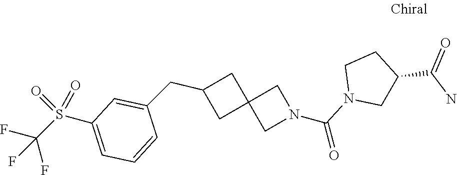 | 460.3 [M + H]⁺ |
| 855 | (3S)-1-[6-[[4-Cyano-3- (trifluoromethyl)phenyl]methyl]- 2-azaspiro[3.3]heptane-2- carbonyl]pyrrolidine-3- carboxamide | 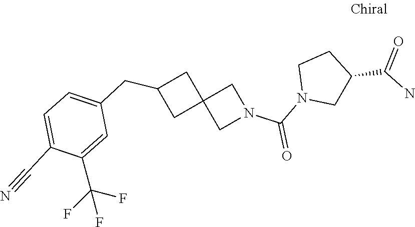 | 421.4 [M + H]⁺ |
| 856 | (3S)-1-[6-[[6- (Trifluoromethoxy)-3- pyridyl]methyl]-2- azaspiro[3.3]heptane-2- carbonyl]pyrrolidine-3- carboxamide | 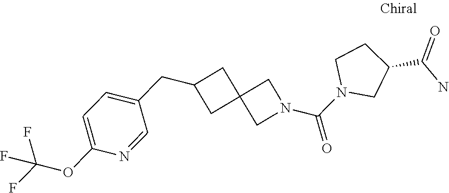 | 413.3 [M + H]⁺ |
| 857 | 5-[[2-[(3S)-3-(1H-1,2,4-Triazol- 5-yl)pyrrolidine-1-carbonyl]-2- azaspiro[3.3]heptan-6- yl]methyl]-2- (trifluoromethyl)benzonitrile | 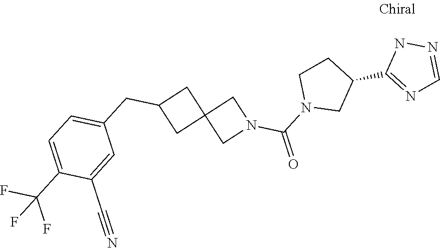 | 445.4 [M + H]⁺ |
| 858 | [(3S)-3-(1H-Triazol-5- yl)pyrrolidin-1-yl]-[6-[[4- (trifluoromethylsulfonimidoyl) phenyl]methyl]-2- azaspiro[3.3]heptan-2- yl]methanone | 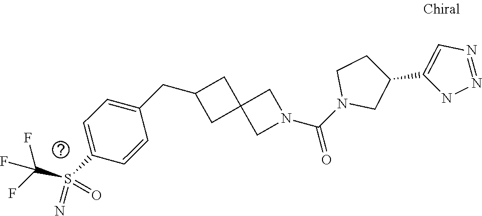 | 483.3 [M + H]⁺ |
| 859 | [(3S)-3-(1H-Triazol-5- yl)pyrrolidin-1-yl]-[6-[[4- (trifluoromethylsulfonimidoyl) phenyl]methyl]-2- azaspiro[3.3]heptan-2- yl]methanone | 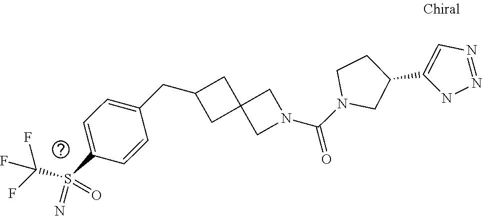 | 483.3 [M + H]⁺ |
| 860 | (3S)-1-[6-[[2-Fluoro-4- (trifluoromethylsulfonimidoyl) phenyl]methyl]-2- azaspiro[3.3]heptane-2- carbonyl]pyrrolidine-3- carboxamide | 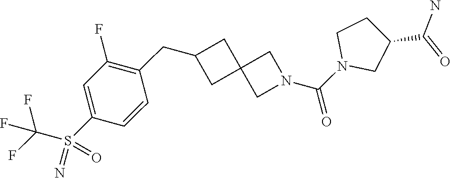 [SMILES: N=S(=O)(c1ccc(CC2CC3(C2)CN(C(=O)N2CC[C@H](C(N)=O)C2)C3)c(F)c1)C(F)(F)F] | 477.3 [M + H]⁺ |
| 861 | [6-[[2-Fluoro-4- (trifluoromethylsulfonimidoyl) phenyl]methyl]-2- azaspiro[3.3]heptan-2-yl]-[(3S)- 3-(1H-triazol-4-yl)pyrrolidin-1- yl]methanone | 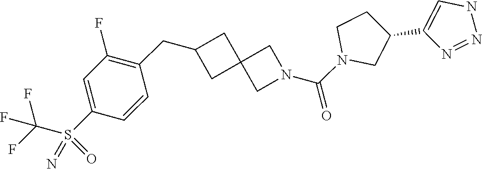 [SMILES: N=S(=O)(c1ccc(CC2CC3(C2)CN(C(=O)N2CC[C@H](c4c[nH]nn4)C2)C3)c(F)c1)C(F)(F)F] | 501.3 [M + H]⁺ |
| 862 | [(3S)-3-(1H-Triazol-4- yl)pyrrolidin-1-yl]-[6-[[4- (trifluoromethylsulfonyl)phenyl] methyl]-2-azaspiro[3.3]heptan- 2-yl]methanone | 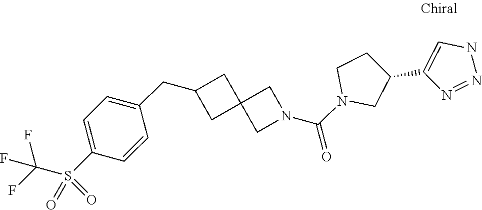 [SMILES: O=C(N1CC[C@H](c2c[nH]nn2)C1)N1CC2(CC(Cc3ccc(S(=O)(=O)C(F)(F)F)cc3)C2)C1] | 484.3 [M + H]⁺ |
| 863 | [6-[[3-Fluoro-5- (trifluoromethylsulfonyl)phenyl] methyl]-2-azaspiro[3.3]heptan- 2-yl]-[(3S)-3-(1H-triazol-4- yl)pyrrolidin-1-yl]methanone | 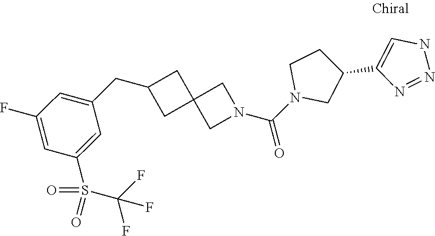 [SMILES: O=C(N1CC[C@H](c2c[nH]nn2)C1)N1CC2(CC(Cc3cc(F)cc(S(=O)(=O)C(F)(F)F)c3)C2)C1] | 502.3 [M + H]⁺ |
| 864 | [(3S)-3-(1H-Triazol-4- yl)pyrrolidin-1-yl]-[6-[[5- (trifluoromethyl)-1H-pyrazol-3- yl]methyl]-2- azaspiro[3.3]heptan-2- yl]methanone | 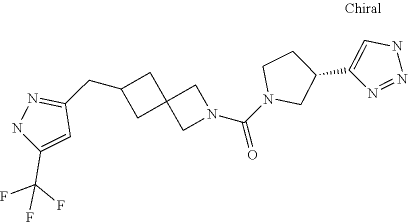 [SMILES: O=C(N1CC[C@H](c2c[nH]nn2)C1)N1CC2(CC(Cc3cc(C(F)(F)F)[nH]n3)C2)C1] | 410.3 [M + H]⁺ |
| 865 | [6-[[2-Fluoro-4- (trifluoromethylsulfonimidoyl) phenyl]methyl]-2- azaspiro[3.3]heptan-2-yl]-[(3S)- 3-(1H-1,2,4-triazol-3- yl)pyrrolidin-1-yl]methanone | 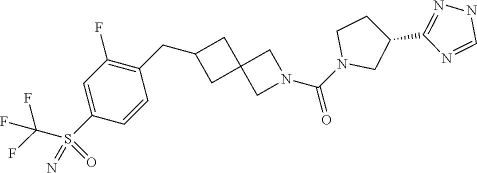 [SMILES: N=S(=O)(c1ccc(CC2CC3(C2)CN(C(=O)N2CC[C@H](c4nc[nH]n4)C2)C3)c(F)c1)C(F)(F)F] | 501.3 [M + H]⁺ |

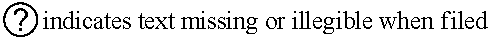

### Synthesis of Building Blocks

### BB 1

### 6-(2-Chloro-4-fluoro-phenoxy)-2-azaspiro[3.3]heptane 2,2,2-trifluoroacetic Acid

Step 1: tert-Butyl 6-(2-chloro-4-fluoro-phenoxy)-2-azaspiro[3.3]heptane-2-carboxylate

To a solution of 2-chloro-4-fluorophenol (756 mg, 562 μl, 5.16 mmol, CAS RN 1996-41-4), tert-butyl 6-hydroxy-2-azaspiro[3.3]heptane-2-carboxylate (1 g, 4.69 mmol, CAS RN 1147557-97-8) and triphenylphosphine (1.48 g, 5.63 mmol) in THF (23.4 ml) was added DIAD (1.09 ml, 5.63 mmol, CAS N 2446-83-5) dropwise at 0° C. and the reaction was stirred at rt for 18 h. Another batch of triphenylphosphine (738 mg, 2.81 mmol), followed by DIAD (547 μl, 2.81 mmol) were added and the reaction was stirred at rt for 6 h. The reaction mixture was poured into sat. aq. NaHCO₃ solution (50 mL) and EtOAc (30 mL) was added. The phases were separated and the aq. phase was extracted with EtOAc. The combined organic layers were washed with brine, dried over sodium sulfate, filtered and concentrated to give an orange oil. The crude was immobilized on Isolute and purified by column chromatography (40 gr, 0->30% EtOAc in hept) to get the desired product as a yellow solid (1.50 g, 89.3% yield). MS (ESI): m/z=286.2 [M-C4H8+H]⁺.

Step 2:6-(2-Chloro-4-fluoro-phenoxy)-2-azaspiro[3.3]heptane 2,2,2-trifluoroacetic acid

To a solution of tert-butyl 6-(2-chloro-4-fluorophenoxy)-2-azaspiro[3.3]heptane-2-carboxylate (1.5065 g, 4.41 mmol) in DCM (22 mL) was added 2,2,2-trifluoroacetic acid (2.72 ml, 35.3 mmol) and the reaction was stirred at rt for 3.5 hours. The reaction mixture was concentrated to to get the desired product as a yellow oil (2.015 g, 100% yield). MS (ESI): m/z=242.2 [M+H]⁺.

### BB 2

### 3-((2-Fluoro-4-(pentafluoro-16-sulfanyl)benzyl)oxy)azetidine 2,2,2-trifluoroacetic acid

Step 1: tert-Butyl 3-((2-fluoro-4-(pentafluoro-16-sulfanyl)benzyl)oxy)azetidine-1-carboxylate

To a solution of tert-butyl 3-hydroxyazetidine-1-carboxylate (5.5 g, 31.8 mmol, CAS RN 141699-55-0) in dry THF (4 ml) was added potassium tert-butoxide 1 M solution in THF (33.3 ml, 33.3 mmol) and the turbid reaction mixture was stirred at r.t for 15 min followed by addition of (4-(bromomethyl)-3-fluorophenyl) pentafluoro-16-sulfane (10 g, 31.8 mmol, CAS RN 1240257-17-3). The reaction mixture was then stirred at r.t for 19 hours. The crude reaction was diluted with EtOAc and extracted with water, the organic phase was collected and the aqueous phase was back-extracted with EtOAc. the combined organic phases were dried over sodium sulfate and evaporated down to dryness. The crude material was purified by flash chromatography (silica gel, 330 g, 0% to 80% EtOAc in heptane): 9.72 g, yellow oil. MS (ESI): m/z=352.1 [M-C4H8+H]⁺.

Step 2:3-((2-Fluoro-4-(pentafluoro-16-sulfanyl)benzyl)oxy)azetidine 2,2,2-trifluoroacetic acid

To a solution of tert-butyl 3-((2-fluoro-4-(pentafluoro-16-sulfanyl)benzyl)oxy)azetidine-1-carboxylate (9.72 g, 23.9 mmol) in DCM (100 ml) was added TFA (27.2 g, 18.4 ml, 239 mmol). The resultant reaction mixture was stirred at RT for 1 hour, then evaporated. The residue was treated with toluene and evaporated to get the desired product as yellow oil (10.1 g, 100%). 308.1 [M+H]⁺.

### BB 3

### (3-(1H-Pyrazol-5-yl)pyrrolidin-1-yl) (3-(4-bromophenyl)azetidin-1-yl)methanone

The product was obtained in analogy to example 1 starting from 3-(4-bromophenyl)azetidine (CAS RN 7215-01-2) and 5-(pyrrolidin-3-yl)-1H-pyrazole dihydrochloride (CAS RN 2173135-18-5) to get the title compound as a colorless gum. MS (ESI): m/z=375.2 [M+H]⁺.

### BB 4

### 6-Pyrrolidin-3-yl-1H-pyrazin-2-one Hydrobromide

To a solution of tert-butyl 3-(6-methoxypyrazin-2-yl)pyrrolidine-1-carboxylate (82 mg, 294 μmol, CAS RN 2355015-85-7) in EtOH (0.3 mL) was added aqueous 48% HBr solution (365 μl, 3.23 mmol) and the RM was heated at 90° C. for 15 hours. The RM was concentrated in vacuo to afford desired compound as a brown solid. MS (ESI): m/z=166.1 [M+H]⁺.

### BB 5

### (+) or (−)-[1-[3-[[2-Fluoro-4-(trifluoromethyl)phenyl]methoxy]azetidine-1-carbonyl]pyrrolidin-3-yl]oxazolidin-2-one and

### BB 6

### (−) or (+)-[1-[3-[[2-Fluoro-4-(trifluoromethyl)phenyl]methoxy]azetidine-1-carbonyl]pyrrolidin-3-yl]oxazolidin-2-one

The diastereomeric racemate of 4-[1-[3-[[2-fluoro-4-(trifluoromethyl)phenyl]methoxy]azetidine-1-carbonyl]pyrrolidin-3-yl]oxazolidin-2-one (example 3) was separated using HPLC Method GN to give the desired product BB5 (first eluting) as a colorless solid (21.3 mg, 4.33%). MS (ESI): m/z=432.2 [M+H]⁺ and the desired product BB6 (second eluting) as a colorless solid (59.5 mg, 12.1%). MS (ESI): m/z=432.2 [M+H]⁺.

### BB 7

### 5-Pyrrolidin-3-ylpyrrolidin-2-one 4-methylbenzenesulfonic acid [ENTITY A]

Step 1: tert-Butyl 3-[1-(benzyloxycarbonylamino)-2-methoxy-2-oxo-ethyl]pyrrolidine-1-carboxylate

To a mixture of tert-butyl 3-(1-amino-2-methoxy-2-oxoethyl)pyrrolidine-1-carboxylate (1.5 g, 5.81 mmol, CAS RN 1135916-70-9) in DCM (15 mL) and NaHCO₃ sat. solution in H2O (10 mL, 5.81 mmol) was added dropwise at RT over 80 minutes under vigorous stirring benzyl carbonochloridate (829 μL, 5.81 mmol). The two-layer mixture was stirred at RT for 20 minutes. The layers were separated. The aqueous layer was extracted twice with DCM. The organic layer was extracted once with water and once with brine, dried over MgSO₄, filtered, treated with silica gel and evaporated. The compound was purified by silica gel chromatography on a 40 g column using an MPLC (ISCO) system eluting with a gradient of n-heptane:EtOAc (100:0 to 30:70) to get the desired compound as a colorless oil (2.06 g; 88.6%). MS (ESI): m/z=293.2 [M-BOC+H]⁺.

Step 2: tert-Butyl 3-[1-(benzyloxycarbonylamino)-2-hydroxy-ethyl]pyrrolidine-1-carboxylate [ENTITY A] and tert-Butyl 3-[1-(benzyloxycarbonylamino)-2-hydroxy-ethyl]pyrrolidine-1-carboxylate [ENTITY B]

To an ice-cold solution of tert-butyl 3-[1-(benzyloxycarbonylamino)-2-methoxy-2-oxo-ethyl]pyrrolidine-1-carboxylate (1.71 g, 4.36 mmol) in THF (19.5 mL) was added dropwise LiBH4 2 M solution in THF (4.36 mL, 8.71 mmol). The clear solution was stirred at RT for 2 hours. The reaction mixture was poured on saturated aqueous sodium potassium tartrate solution and EtOAc and the layers were separated. The aqueous layer was extracted twice with EtOAc. The organic layers were washed with water, dried over MgSO₄, filtered, treated with silica gel and evaporated. The compound was purified three times by silica gel chromatography on a 80 g column using an MPLC system eluting with a gradient of n-heptane:EtOAc (100:0 to 0:100) to get the desired products [ENTITY A] (first eluting) and [ENTITY B] (second eluting). MS (ESI): m/z=265.2 [M-C4H8+H]⁺.

Step 3: tert-Butyl 3-[1-(benzyloxycarbonylamino)-2-oxo-ethyl]pyrrolidine-1-carboxylate [ENTITY A]

To an ice-cold solution of tert-butyl 3-[1-(benzyloxycarbonylamino)-2-hydroxy-ethyl]pyrrolidine-1-carboxylate (660 mg, 1.81 mmol, [ENTITY A]) in DCM (15 mL) was added DMP (1.08 g, 2.54 mmol, CAS RN 87413-09-0) and the mixture was stirred at 0° C. for 1.75 hours. The reaction mixture was diluted with DCM and aqueous 2 M Na2CO3 solution and the layers were separated. The aqueous layer was extracted twice with DCM. The organic layers were washed with 10% aqueous sodium thiosulfate solution and brine, dried over MgSO₄, filtered, treated with silica gel and evaporated. The compound was purified by silica gel chromatography on a 12 g column using an MPLC system eluting with a gradient of n-heptane:EtOAc (100:0 to 0:100) to get the desired compound as a colorless gum (0.530 g; 80.7%). MS (ESI): m/z=263.1 [M-BOC+H]⁺.

Step 4: tert-Butyl 3-[(E)-1-(benzyloxycarbonylamino)-4-ethoxy-4-oxo-but-2-enyl]pyrrolidine-1-carboxylate [ENTITY A]

To an ice-cold solution of ethyl 2-(diethoxyphosphoryl)acetate (290 μL, 1.46 mmol) in THF (5 mL) was added sodium hydride 55% in mineral oil (63.8 mg, 1.46 mmol) and the mixture was stirred at this temperature for 30 minutes. A solution of tert-butyl 3-[1-(benzyloxycarbonylamino)-2-oxo-ethyl]pyrrolidine-1-carboxylate (530 mg, 1.46 mmol) in THF (11 mL) was added dropwise. Stirring was continued at ice-bath temperature for 1.5 hours. The reaction mixture was poured on water and EtOAc and the layers were separated. The aqueous layer was extracted twice with EtOAc. The organic layers were dried over MgSO₄, filtered, treated with silica gel and evaporated. The compound was purified by silica gel chromatography on a 12 g column using an MPLC system eluting with a gradient of n-heptane:EtOAc (100:0 to 50:50) to get the desired compound as a colorless gum (0.519 g; 73.8%). MS (ESI): m/z=377.2 [M-C4H8+H]⁺.

Step 5: tert-Butyl 3-(5-oxopyrrolidin-2-yl)pyrrolidine-1-carboxylate [ENTITY A]

A mixture of tert-butyl 3-[(E)-1-(benzyloxycarbonylamino)-4-ethoxy-4-oxo-but-2-enyl]pyrrolidine-1-carboxylate (205 mg, 474 μmol) and ammonium formate (598 mg, 9.48 mmol) in MeOH (4 mL) under argon was treated with Pd—C 10% (5.04 mg, 47.4 μmol) and stirred at reflux in a sealed tube for 4.5 hours. The reaction mixture was filtered. The filtrate was evaporated. The residue was taken up in diluted aqueous NaHCO₃ solution and EtOAc and the layers were separated. The aqueous layer was extracted twice with EtOAc. The organic layers were dried over MgSO₄, filtered and evaporated to get the desired compound as a colorless solid (0.112 g; 92.9%). MS (ESI): m/z=199.1 [M-C4H8+H]⁺.

Step 6:5-Pyrrolidin-3-ylpyrrolidin-2-one 4-methylbenzenesulfonic acid [ENTITY A]

A suspension of tert-butyl 3-(5-oxopyrrolidin-2-yl)pyrrolidine-1-carboxylate (75 mg, 295 μmol) and p-toluenesulfonic acid monohydrate (67.3 mg, 354 μmol) in EtOAc (0.8 mL) was heated at reflux for 1 hour, followed by stirring overnight at RT. The mixture was homogenized in an ultrasonic filled with ice-water. The suspension was filtered. The filter cake was washed with EtOAc to get the desired compound as a light yellow solid (0.085 g; 72.4%). MS (ESI): m/z=155.1 [M+H]⁺.

### BB 8

### 5-Pyrrolidin-3-ylpyrrolidin-2-one 4-methylbenzenesulfonic acid [ENTITY B]

The product was obtained in analogy to BB7, steps 3 to 6 starting from tert-butyl 3-[1-(benzyloxycarbonylamino)-2-hydroxy-ethyl]pyrrolidine-1-carboxylate [ENTITY B] (BB7, step 2) to get the title compound as a colorless solid. MS (ESI): m/z=155.1 [M+H]⁺.

### BB 9

### 3-[4-[1-(Trifluoromethyl)cyclopropyl]phenyl]azetidine 4-methylbenzenesulfonic Acid

Step 1: tert-Butyl 3-[4-[1-(trifluoromethyl)cyclopropyl]phenyl]azetidine-1-carboxylate

To a 20 mL vial, equipped with a stir bar, was added 1-bromo-4-(1-(trifluoromethyl)cyclopropyl)benzene (561 mg, 2.12 mmol, 1.0 equiv; CAS RN 1227160-18-0), tert-butyl 3-iodoazetidine-1-carboxylate (600 mg, 2.12 mmol, 1.0 equiv; CAS RN 254454-54-1), tris(trimethylsilyl) silane (527 mg, 653 μL, 2.12 mmol, 1.0 equiv), photocatalyst bis[3,5-difluoro-2-[5-(trifluoromethyl)-2-pyridyl]phenyl]iridium (1+) 4-tert-butyl-2-(4-tert-butyl-2-pyridyl)pyridine hexafluorophosphate (23.8 mg, 21.2 μmol, 0.01 equiv;

Ir[dF(CF3)ppy]₂(dtbbpy))PF₆, CAS RN 870987-63-6) and anhydrous sodium carbonate (449 mg, 4.24 mmol, 2.0 equiv). The vial was sealed and placed under Ar before dimethoxyethane (9 mL) was added. To a separate vial was added nickel (II) chloride ethylene glycol dimethyl ether complex (4.65 mg, 21.2 μmol, 0.01 equiv; CAS RN 29046-78-4) and 4,4′-di-tert-butyl-2,2′-bipyridine (5.68 mg, 21.2 μmol, 0.01 equiv). The vial was sealed, purged with Ar, and dimethoxyethane (4 mL) was added. The precatalyst solution was sonicated for 5 min, after which 2 mL were syringed into the reaction vessel. The reaction mixture was degassed with Ar and irradiated with a blue LED lamp (420 nm) for 1 h. The reaction was quenched by exposure to air, filtered and the solvent evaporated. The crude reaction mixture was purified by silica gel chromatography using an MPLC system eluting with a gradient of n-heptane:EtOAc (100:0 to 70:30) to furnish the title compound as a colorless solid (0.51 g, 66%). MS (ESI): m/z=286.1 [M-C4H8+2H]⁺.

Step 2:3-[4-[1-(Trifluoromethyl)cyclopropyl]phenyl]azetidine 4-methylbenzenesulfonic acid

To a solution of tert-butyl 3-[4-[1-(trifluoromethyl)cyclopropyl]phenyl]azetidine-1-carboxylate (0.5 g, 1.46 mmol, 1.0 equiv) in EtOAc (5 mL) was added 4-methylbenzenesulfonic acid hydrate (0.29 g, 1.54 mmol, 1.1 equiv) and the mixture was heated at reflux for 2 h. The suspension was cooled in the fridge at 0° C. for 1 h and the filtered. The precipitate was washed with EtOAc and dried to yield the title compound as a colorless solid (0.52 g, 82%). MS (ESI): m/z=242.2 [M+H]⁺.

### BB 10

### 6-(2,4-Difluorobenzyl)-2-azaspiro[3.3]heptane 2,2,2-trifluoroacetic Acid

### Step 1:(2,4-Difluorobenzyl)triphenylphosphonium Bromide

Under argon, triphenylphosphine (2.53 g, 9.66 mmol) was dissolved in acetonitrile (25 ml) and 1-(bromomethyl)-2,4-difluorobenzene (2.00 g, 1.24 ml, 9.66 mmol, CAS RN 23915-07-3) was added. The mixture was stirred at 80° C. for 1 hour. The suspension was allowed to cool to RT. It was added 250 mL TBME and was stirred at Rt for 30 min. The solid was filtrated and washed with TBME to get the desired product as a colorless solid (4.5 g; 100%). MS (ESI): m/z=389.2 [M−Br+H]⁺.

Step 2: tert-Butyl 6-(2,4-difluorobenzylidene)-2-azaspiro[3.3]heptane-2-carboxylate

Under Argon at −78° C., (2,4-difluorobenzyl)triphenylphosphonium bromide (4.6 g, 9.8 mmol) was dissolved in dry THF (46 ml) and LHMDS 1 M in THF (19.6 ml, 19.6 mmol) was added. The reaction mixture was stirred at −78° C. for 2 hours. Then at RT, tert-butyl 6-oxo-2-azaspiro[3.3]heptane-2-carboxylate (4.14 g, 19.6 mmol, CAS RN 1181816-12-5) was added and the mixture was stirred at 85° C. overnight. After cooling down, TBME was added. The suspension was filtered. The filtrate was concentrated then purified by flash chromatography (silica gel, 100 g, 0% to 80% EtOAc in heptane). The residue was treated with toluene and evaporated to get the desired product as yellow oil (10.1 g, 100%). 308.1 [M+H]⁺.

Step 3: tert-Butyl 6-[(2,4-difluorophenyl)methyl]-2-azaspiro[3.3]heptane-2-carboxylate

To a solution of tert-butyl 6-(2,4-difluorobenzylidene)-2-azaspiro[3.3]heptane-2-carboxylate (1.66 g, 5.17 mmol) was dissolved in EtOAc (30 mL). The flask was purged and backfilled with argon (×3). Pd—C (550 mg, 517 μmol, Eq: 0.1) was added and the reaction was stirred under a hydrogen balloon for 3 hours. The reaction mixture was filtered through celite pad, washed with EtOAc and dried under vacuum to get the desired crude product (1.44 g, 86.2%). 268.2 [M-C4H8+H]⁺.

Step 4:6-(2,4-Difluorobenzyl)-2-azaspiro[3.3]heptane 2,2,2-trifluoroacetic acid

To a solution of tert-butyl 6-[(2,4-difluorophenyl)methyl]-2-azaspiro[3.3]heptane-2-carboxylate (1.44 g, 4.45 mmol) in DCM (28.8 mL) was added TFA (1.72 mL, 22.3 mmol). The resultant reaction mixture was stirred at RT for 2 hours. The mixture was evaporated to get the desired crude product as a colorless oil (1.8 g). 224.1 [M+H]⁺.

### BB 11

### 2-[2-Fluoro-4-(trifluoromethyl) phenoxy]-7-azaspiro[3.5]nonane 2,2,2-trifluoroacetic Acid

Step 1: tert-Butyl 2-[2-fluoro-4-(trifluoromethyl) phenoxy]-7-azaspiro[3.5]nonane-7-carboxylate

To a solution of tert-butyl 2-hydroxy-7-azaspiro[3.5]nonane-7-carboxylate (442 mg, 1.83 mmol, CAS RN 240401-28-9) in THF (8 mL) was added 2-fluoro-4-(trifluoromethyl) phenol (330 mg, 1.83 mmol, CAS RN 77227-78-2) and triphenylphosphine (529 mg, 2.02 mmol, Eq: 1.1). After stirring at RT for 5 minutes at RT, the solution was cooled down in an ice-bath and DEAD (351 mg, 319 μl, 2.02 mmol, Eq: 1.1) was added dropwise over 10 minutes. After stirring for 1 hour in the ice-bath the mixture was allowed to stir at RT for 5 hours. The reaction mixture was poured on water and diethylether and the layers were separated. The organic layer was washed with water, aqueous 1 M NaOH solution and brine, dried over MgSO₄, filtered and evaporated. The compound was purified by silica gel chromatography on a 25 g column using an MPLC system eluting with a gradient of n-heptane:EtOAc (100:0 to 60:40) to get the desired compound as a colorless solid (0.625 g; 83.9%). MS (ESI): m/z=348.1 [M-C4H8+H]⁺.

Step 2:2-[2-Fluoro-4-(trifluoromethyl) phenoxy]-7-azaspiro[3.5]nonane 2,2,2-trifluoroacetic Acid

To a solution of tert-butyl 2-[2-fluoro-4-(trifluoromethyl) phenoxy]-7-azaspiro[3.5]nonane-7-carboxylate (70 mg, 174 μmol) in DCM (1 mL) was added TFA (66.8 μl, 868 μmol) and the mixture was stirred at RT for 20 hours. The solution was evaporated to get the desired product as colorless solid (0.073 g, 100%). 304.2 [M+H]⁺.

### BB12

### 3-[4-(2,4-Difluorophenyl)phenyl]azetidine 4-methylbenzenesulfonic Acid

Step 1: tert-Butyl 3-[4-(2,4-difluorophenyl)phenyl]azetidine-1-carboxylate

A suspension of tert-butyl 3-(4-bromophenyl)azetidine-1-carboxylate (1.3 g, 4.16 mmol, CAS RN 1203681-52-0), (2,4-difluorophenyl) boronic acid (658 mg, 4.16 mmol, CAS RN 144025-03-6), potassium carbonate (2.88 g, 20.8 mmol), Water (1 ml) and tetrakis (triphenylphosphine)palladium (0) (241 mg, 208 μmol) in THF (10 ml) were heated via microwave radiation at 110° C. for 15 mins. The crude was product was then poured over EtOAc and water. The aqueous layer was extracted by EtOAc (×3) and the solvent removed in vacuo. The crude product was purified by flash chromatography (Hept:EE 100:0 to 50:50). Product fraction was dried in vacuo, yielding product as yellow oil (1.20; 78.7%). MS (ESI): m/z=290.2 [M−C4H8+H]⁺.

Step 2:3-[4-(2,4-Difluorophenyl)phenyl]azetidine 4-methylbenzenesulfonic Acid

The product was obtained in analogy to BB9, step 2 starting from tert-butyl 3-[4-(2,4-difluorophenyl)phenyl]azetidine-1-carboxylate to get the title compound as a colorless solid. MS (ESI): m/z=246.2 [M+H]⁺.

### BB13

### Trans-3-[[2-Fluoro-4-(trifluoromethyl)phenyl]methoxy]cyclobutanecarboxylic Acid

Step 1: trans-Methyl 3-[[2-fluoro-4-(trifluoromethyl)phenyl]methoxylcyclobutanecarboxylate

To an ice-cold solution of methyl 3-hydroxycyclobutanecarboxylate (506 mg, 3.89 mmol, CAS RN 63485-51-8) in DMF (8.33 mL) was added sodium hydride 55% in mineral oil (187 mg, 4.28 mmol) and the mixture was stirred at this temperature for 30 minutes. A solution of 1-(bromomethyl)-2-fluoro-4-(trifluoromethyl)benzene (1 g, 3.89 mmol, CAS RN 239087-07-1) in DMF (1.67 mL) was added dropwise to the mixture at 0° C. Stirring of the slurry was continued at RT for 3 hours. The reaction mixture was poured on saturated aqueous NH₄Cl solution (300 mL) and EtOAc (300 mL) and the layers were separated. The aqueous layer was extracted once with EtOAc (100 mL). The organic layers were washed twice with water, dried over MgSO₄, filtered and evaporated to get the desired product as a colorless solid. MS (ESI): m/z=307.1 [M+H]⁺. Used as is for next step.

Step 2: trans-3-[[2-Fluoro-4-(trifluoromethyl)phenyl]methoxy]cyclobutanecarboxylic Acid

To a solution of methyl 3-[[2-fluoro-4-(trifluoromethyl)phenyl]methoxy]cyclobutanecarboxylate (730 mg, 2.38 mmol) in THF (10 mL), MeOH (10 mL) and water (10 mL) was added LiOH monohydrate (500 mg, 11.9 mmol) and the mixture was stirred at RT overnight. The reaction mixture was poured then onto water and the pH was set to 2 using aqueous 1 M HCl solution. The aqueous layer was extracted twice with EtOAc. The organic layers were dried over Na2SO4, filtered and evaporated to get the desired product as a light yellow liquid (646 mg, 63.1%). MS (ESI): m/z=291.2 [M−H]⁻.

### BB 14

### [3-(1H-Pyrazol-5-yl)pyrrolidin-1-yl]-[3-(4-tetrahydropyran-4-ylphenyl)azetidin-1-yl]methanone

Step 1: tert-Butyl 3-(4-tetrahydropyran-4-ylphenyl)azetidine-1-carboxylate

The product was obtained in analogy to BB9, step 1 starting from tert-butyl 3-(4-bromophenyl)azetidine-1-carboxylate (CAS RN 1203681-52-0) and 4-bromo-tetrahydropyran (CAS N 25637-16-5) to get the title compound as a light yellow solid. MS (ESI): m/z=262.0 [M−C4H8+H]⁺.

Step 2:3-(4-Tetrahydropyran-4-ylphenyl)azetidine Hydrochloride

A solution of tert-butyl 3-(4-tetrahydropyran-4-ylphenyl)azetidine-1-carboxylate (500.0 mg, 1.58 mmol) in 4 M HCl in EtOAC (10.0 mL, 40 mmol) was stirred at RT for 4 hours. The solution was concentrated in vacuo to give the desired product as a yellow oil (399 mg, 74.8%). MS (ESI): m/z=: 218.5 [M+H]⁺.

Step 3: (4-Nitrophenyl) 3-(4-tetrahydropyran-4-ylphenyl)azetidine-1-carboxylate

A solution of 3-(4-tetrahydropyran-4-ylphenyl)azetidine hydrochloride (150.0 mg, 0.590 mmol), 4-nitrophenyl chloroformate (120.0 mg, 0.600 mmol, CAS RN 7693-46-1) and TEA (0.2 mL, 1.43 mmol) in DCM (4 mL) was stirred at RT for 3 hours. The solution was concentrated in vacuo to give a residue, which was purified by flash column (petroleum ether:EtOAc=3:1) to give the desired product as a colorless oil (170 mg, 74.4%). MS (ESI): m/z=383.3 [M+H]⁺.

Step 4: [3-(1H-Pyrazol-5-yl)pyrrolidin-1-yl]-[3-(4-tetrahydropyran-4-ylphenyl)azetidin-1-yl]methanone

The product was obtained in analogy to example 8 starting from (4-nitrophenyl) 3-(4-tetrahydropyran-4-ylphenyl)azetidine-1-carboxylate and 5-(pyrrolidin-3-yl)-1H-pyrazole hydrochloride (CAS RN 1956389-94-8) to get the title compound as a colorless oil. MS (ESI): m/z=381.1 [M+H]⁺.

If not indicated otherwise the following building blocks were synthesized in analogy to the synthesis described for BB14 using suitable building blocks, respectively.

| | | Steps (analogy to BB14 synthesis) | MS |
|---|---|---|---|
| BB | Systematic Name | Building block | m/z |
| 15 | [3-(4- | steps 3 + 4 | 355.1 |
| | Isopropoxyphenyl)azetidin-1- | 3-(4-Isopropoxyphenyl)azetidine | [M + H]⁺ |
| | yl]-[3-(1H-pyrazol-5- | hydrochloride (CAS N 180370-42- | |
| | yl)pyrrolidin-1-yl]methanone | 2) | |
| 16 | [3-(4-Propoxyphenyl)azetidin-1- | steps 3 + 4 | 355.1 |
| | yl]-[3-(1H-pyrazol-5- | 3-(4-Propoxyphenyl)azetidine | [M + H]⁺ |
| | yl)pyrrolidin-1-yl]methanone | (CAS N 1260797-70-3) | |
| 17 | [3-(1H-Pyrazol-5-yl)pyrrolidin- | steps 1-4 | 367.2 |
| | 1-yl]-[3-(4-THF-3- | 3-Bromo-THF (CAS RN 19311-37- | [M + H]⁺ |
| | ylphenyl)azetidin-1- | 6) | |
| | yl]methanone | | |
| 18 | [3-(1H-Pyrazol-5-yl)pyrrolidin- | steps 1-4 | 381.3 |
| | 1-yl]-[3-(4-tetrahydropyran-3- | 3-Bromo-tetrahydropyran (CAS RN | [M + H]⁺ |
| | ylphenyl)azetidin-1- | 13047-01-3) | |
| | yl]methanone | | |
| 22 | (4-Nitrophenyl) 3-[4-(oxetan-3- | steps 1-3 | 355.3 |
| | yl)phenyl]azetidine-1- | 4-Bromo-oxetane (CAS N 39267- | [M + H]⁺ |
| | carboxylate | 79-3) | |
| 23 | [3-(3,3-Dimethyl-2H- | steps 3 + 4 | 367.2 |
| | benzofuran-6-yl)azetidin-1-yl]- | 3-(3,3-Dimethyl-2H-benzofuran-6- | [M + H]⁺ |
| | [3-(1H-pyrazol-5-yl)pyrrolidin- | yl)azetidine (CAS RN 2275677-81- | |
| | 1-yl]methanone | 9) | |

### BB 19

### [3-[4-[(1R,5S)-8-Oxa-3-azabicyclo[3.2.1]octan-3-yl]phenyl]azetidin-1-yl]-[3-(1H-pyrazol-5-yl)pyrrolidin-1-yl]methanone

Step 1: tert-Butyl 3-[4-[(1R,5S)-8-oxa-3-azabicyclo[3.2.1]octan-3-yl]phenyl]azetidine-1-carboxylate

To a solution of X-PHOS (152.7 mg, 0.320 mmol, CAS RN 564483-18-7) and sodium tert-butoxide (307.82 mg, 3.2 mmol) in DME (20 mL) were added Pd2(dba)3 (293.31 mg, 0.320 mmol), tert-butyl 3-(4-bromophenyl)azetidine-1-carboxylate (500.0 mg, 1.6 mmol, CAS RN 1203681-52-0) and 8-oxa-3-azabicyclo[3.2.1]octane hydrochloride (239.62 mg, 1.6 mmol, CAS RN 54745-74-3) and the mixture was stirred under N₂ atmosphere at 110° C. for 12 hours. The mixture was diluted with water (20 mL) and extracted with DCM (20 mL×3). The combined organic layers were concentrated under reduced pressure. The residue was purified by silica gel chromatography (petroleum ether:EtOAc=3:1) to afford the desired product as a light yellow oil (280 mg, 49.2%). MS (ESI): m/z=345.1 [M+H]⁺

Step 2: rac-(1R,5S)-3-[4-(Azetidin-3-yl)phenyl]-8-oxa-3-azabicyclo[3.2.1]octane 2,2,2-trifluoroacetic Acid

The product was obtained in analogy to BB14, step 2 starting from tert-butyl 3-[4-[(1R,5S)-8-oxa-3-azabicyclo[3.2.1]octan-3-yl]phenyl]azetidine-1-carboxylate and TFA to get the title compound as a light yellow oil. MS (ESI): m/z=245.1 [M+H]⁺.

Step 3: (4-Nitrophenyl) 3-[4-[(1R,5S)-8-oxa-3-azabicyclo[3.2.1]octan-3-yl]phenyl]azetidine-1-carboxylate

The product was obtained in analogy to BB14, step 3 starting from rac-(1R,5S)-3-[4-(Azetidin-3-yl)phenyl]-8-oxa-3-azabicyclo[3.2.1]octane 2,2,2-trifluoroacetic acid to get the title compound as a yellow oil. Used as is for next step.

Step 4: [3-[4-[(1R,5S)-8-Oxa-3-azabicyclo[3.2.1]octan-3-yl]phenyl]azetidin-1-yl]-[3-(1H-pyrazol-5-yl)pyrrolidin-1-yl]methanone

The product was obtained in analogy to example 8 starting from (4-nitrophenyl) 3-[4-[(1R,5S)-8-oxa-3-azabicyclo[3.2.1]octan-3-yl]phenyl]azetidine-1-carboxylate and 5-(pyrrolidin-3-yl)-1H-pyrazole hydrochloride (CAS RN 1956389-94-8) to get the title compound as a colorless oil. MS (ESI): m/z=408.3 [M+H]⁺.

### BB 20

### (4-Nitrophenyl) 3-[4-(2-methylazetidin-1-yl)phenyl]azetidine-1-carboxylate

The product was obtained in analogy to BB19, steps 1 to 3 starting from tert-butyl 3-(4-bromophenyl)azetidine-1-carboxylate (CAS RN 1203681-52-0) and 2-methylazetidine hydrochloride to get the title compound as a brown solid. MS (ESI): m/z=368.3 [M-C4H8+H]⁺.

### BB 21

### (4-Nitrophenyl) 3-[4-(3-fluoropyrrolidin-1-yl)phenyl]azetidine-1-carboxylate

Step 1: tert-Butyl 3-[4-(3-hydroxypyrrolidin-1-yl)phenyl]azetidine-1-carboxylate

The product was obtained in analogy to BB19, step 1 starting from tert-butyl 3-(4-bromophenyl)azetidine-1-carboxylate (CAS RN 1203681-52-0) and pyrrolidin-3-ol to get the title compound as a light yellow solid. MS (ESI): m/z=319.4 [M+H]⁺.

Step 2: tert-Butyl 3-[4-(3-fluoropyrrolidin-1-yl)phenyl]azetidine-1-carboxylate

To a solution of tert-butyl 3-[4-(3-hydroxypyrrolidin-1-yl)phenyl]azetidine-1-carboxylate (130.0 mg, 0.410 mmol) in DCM (5 mL) was added diethylaminosulfur trifluoride (100.0 mg, 0.620 mmol). The reaction solution was stirred at 25° C. for 16 hours. The solution was poured into brine (10 mL) and extracted with DCM (10 mL). The organic layer was concentrated in vacuo to give a residue, which was purified by flash column (petroleum ether:EtOAc=10:1) to get the title compound as a colorless solid (105 mg, 80.2%). MS (ESI): m/z=321.1 [M+H]⁺.

Step 3:1-[4-(Azetidin-3-yl)phenyl]-3-fluoro-pyrrolidine 2,2,2-trifluoroacetic Acid

The product was obtained in analogy to BB14, step 2 starting from tert-butyl 3-[4-(3-fluoropyrrolidin-1-yl)phenyl]azetidine-1-carboxylate and TFA to get the title compound as a colorless oil. MS (ESI): m/z=221.1 [M+H]⁺.

Step 4: (4-Nitrophenyl) 3-[4-(3-fluoropyrrolidin-1-yl)phenyl]azetidine-1-carboxylate

The product was obtained in analogy to BB14, step 3 starting from 1-[4-(azetidin-3-yl)phenyl]-3-fluoro-pyrrolidine 2,2,2-trifluoroacetic acid to get the title compound as a brown solid. MS (ESI): m/z=386.1 [M-C4H8+H]⁺.

### BB 24

### 3-(4-((1,1,1-Trifluoro-2-methylpropan-2-yl)oxy)phenyl)azetidine 4-methylbenzenesulfonic Acid

Step 1:1-Nitro-4-((1,1,1-trifluoro-2-methylpropan-2-yl)oxy)benzene

To an ice-cold solution of 1,1,1-trifluoro-2-methylpropan-2-ol (327 mg, 2.55 mmol, 1.2 equiv; CAS RN507-52-8) in DMF (4 mL) was added sodium hydride (102 mg, 2.55 mmol, 1.2 equiv; 55% in mineral oil) and the reaction mixture stirred at RT. After 1 h, 1-fluoro-4-nitrobenzene (300 mg, 2.13 mmol, 1.0 equiv; CAS RN350-46-9) was added portionwise and stirring was continued overnight at RT. The reaction mixture was poured on half-saturated aqueous NH₄Cl solution and EtOAc and the layers were separated. The aqueous layer was extracted twice with EtOAc. The combined organic layers were washed three times with water, dried over MgSO₄, filtered, treated with silica gel and evaporated. The compound was purified by silica gel chromatography using an MPLC system eluting with a gradient of n-heptane:EtOAc (100:0 to 50:50) to get the desired compound as a colorless oil (0.42 g, 75%), which was used without further purification in the consecutive step.

Step 2:4-((1,1,1-Trifluoro-2-methylpropan-2-yl)oxy)aniline

To a solution of 1-nitro-4-((1,1,1-trifluoro-2-methylpropan-2-yl)oxy)benzene (420 mg, 1.69 mmol, 1.0 equiv) in EtOAc (2 mL) and MeOH (2 mL) was added Pd/C (39.5 mg, 37.1 μmol, 0.022 equiv; wt. 10%) and the suspension was stirred at RT for 5 h under an atmosphere of H₂ (balloon). The suspension was filtered and the filtrate evaporated to get the desired compound as a colorless solid (0.36 g, 92%). MS (ESI): m/z=220.2 [M+H]⁺.

Step 3:1-Bromo-4-((1,1,1-trifluoro-2-methylpropan-2-yl)oxy)benzene

To a solution of 4-((1,1,1-trifluoro-2-methylpropan-2-yl)oxy)aniline (360 mg, 1.64 mmol, 1.0 equiv) in acetonitrile (8 mL) was added copper (II) bromide (477 mg, 2.13 mmol, 1.3 equiv). The dark suspension was heated to 60° C. At this temperature tert-butyl nitrite (220 mg, 2.13 mmol, 1.3 equiv) was added dropwise and the mixture was stirred at 70° C. overnight. After cooling down, the dark mixture was poured on a sat. aqueous NaHCO₃ solution. The aqueous layer was extracted twice with EtOAc. The combined organic layers were dried over MgSO₄, filtered, treated with silica gel and evaporated. The compound was purified by silica gel chromatography using an MPLC system eluting with a gradient of n-heptane:EtOAc (100:0 to 50:50) to get the desired compound as a colorless oil (0.38 g, 40%). Used as is for next step.

Step 4: tert-Butyl 3-(4-((1,1,1-trifluoro-2-methylpropan-2-yl)oxy)phenyl)azetidine-1-carboxylate

The product was obtained in analogy to BB 4, step 1 starting from 1-bromo-4-((1,1,1-trifluoro-2-methylpropan-2-yl)oxy)benzene to get the desired compound as a light brown solid (0.10 g, 20%). MS (ESI): m/z=304.2 [M+2H-tBu]⁺.

Step 5:3-(4-((1,1,1-Trifluoro-2-methylpropan-2-yl)oxy)phenyl)azetidine 4-methylbenzenesulfonic Acid

To a solution of tert-butyl 3-(4-((1,1,1-trifluoro-2-methylpropan-2-yl)oxy)phenyl)azetidine-1-carboxylate (98 mg, 0.273 mmol, 1.0 equiv) in EtOAc (1 mL) was added 4-methylbenzenesulfonic acid hydrate (55 mg, 0.286 mmol, 1.1 equiv) and the mixture was heated at reflux for 1.5 h. The suspension was cooled in the fridge at 0° C. for 30 min and filtered. The precipitate was washed with EtOAc and dried to yield the title compound as a colorless solid (81 mg, 65%). MS (ESI): m/z=260.2 [M+H]⁺.

### BB25

### 5-[(3S or 3R)-Pyrrolidin-3-yl]-1H-pyrazole and

### BB26

### 5-[(3R or 3S)-Pyrrolidin-3-yl]-1H-pyrazole

5-Pyrrolidin-3-yl-1H-pyrazole hydrochloride (300.0 mg, 1.73 mmol, CAS RN 1956389-94-8) was separated by SFC Method ADH2 to give peak 1 (BB25) as a light yellow oil (80 mg, 30.38% yield) MS (ESI): m/z=138.1 [M+H]⁺ and peak 2 as a light yellow oil (BB26) (82 mg, 29.75% yield). MS (ESI): m/z=138.1 [M+H]⁺.

### BB 27

### (4-Nitrophenyl) 3-(1H-triazol-5-yl)pyrrolidine-1-carboxylate

The product was obtained in analogy to BB14, step 3 starting from 5-pyrrolidin-3-yl-1H-triazole (CAS RN 1517602-91-3) to get the title compound as a yellow gum. Used as is for next step.

### BB28

### (4-Nitrophenyl) (3S or 3R)-3-(1H-triazol-5-yl)pyrrolidine-1-carboxylate and

### BB29

### (4-Nitrophenyl) (3R or 3S)-3-(1H-triazol-5-yl)pyrrolidine-1-carboxylate

The racemate (4-nitrophenyl) 3-(1H-triazol-5-yl)pyrrolidine-1-carboxylate (BB27) was separated using SFC Method AD6 to get the desired product BB28 as yellow gum. MS (ESI): m/z=304.1 [M+H the desired product BB29 as yellow gum. MS (ESI): m/z=304.1 [M+H]⁺.

### BB28. Specific Rotation: −20.145° BB29. Specific Rotation: +23.642°

### BB30

### 4-[[2-Chloro-4-(trifluoromethyl)phenyl]methoxy]piperidine 2,2,2-trifluoroacetic Acid

Step 1: tert-Butyl 4-[[2-chloro-4-(trifluoromethyl)phenyl]methoxy]piperidine-1-carboxylate

To a solution of 1-BOC-4-hydroxypiperidine (397 mg, 1.97 mmol, CAN RN 109384-19-2) in THF (9 mL) was added t-BuOK (277 mg, 2.47 mmol) and stirred at 25° C. for 30 min. Then 1-(bromomethyl)-2-chloro-4-(trifluoromethyl)benzene (450 mg, 1.65 mmol, CAS RN 279252-26-5) was added in the mixture and stirred at 25° C. for 12 h. The mixture was poured into aq. NH4Cl (30 mL) and extracted with EtOAc (20 mL, three times), the combined organic phase was washed with brine, dried over Na2SO4, concentrated and purified by reverse flash (0.05% v/v FA condition) to give the desired product as a yellow oil (228 mg, 35.18% yield). MS (ESI): m/z=338.1 [M−C4H8+H]⁺.

Step 2:4-[[2-Chloro-4-(trifluoromethyl)phenyl]methoxy]piperidine 2,2,2-trifluoroacetic Acid

The product was obtained in analogy to BB14, step 2 starting from tert-butyl 4-[[2-chloro-4-(trifluoromethyl)phenyl]methoxy]piperidine-1-carboxylate and TFA to get the title compound as a light yellow oil. MS (ESI): m/z=294.1 [M+H]⁺.

### BB31

### 3-[[2-Fluoro-4-(trifluoromethyl)phenyl]methoxy]-8-azabicyclo[3.2.1]octane 2,2,2-trifluoroacetic Acid

Step 1: tert-Butyl 3-[[2-fluoro-4-(trifluoromethyl)phenyl]methoxy]-8-azabicyclo[3.2.1]octane-8-carboxylate

The product was obtained in analogy to BB30, step 1 starting from tert-butyl 3-hydroxy-8-azabicyclo[3.2.1]octane-8-carboxylate (CAS RN 478837-18-2) and 1-(bromomethyl)-2-chloro-4-(trifluoromethyl)benzene (CAS RN 279252-26-5) to get the title compound as yellow oil. MS (ESI): m/z=348.1 [M-C4H8+H]⁺.

Step 2:3-[[2-Fluoro-4-(trifluoromethyl)phenyl]methoxy]-8-azabicyclo[3.2.1]octane 2,2,2-trifluoroacetic Acid

The product was obtained in analogy to BB14, step 2 starting from tert-butyl 3-[[2-fluoro-4-(trifluoromethyl)phenyl]methoxy]-8-azabicyclo[3.2.1]octane-8-carboxylate and TFA to get the title compound as red oil. MS (ESI): m/z=304.1 [M+H]⁺.

### BB32

### Rac-(3aS,6aR)-5-[2-Fluoro-4-(trifluoromethyl) phenoxy]-1,2,3,3a,4,5,6,6a-octahydrocyclopenta[c]pyrrole 2,2,2-trifluoroacetic Acid

Step 1: tert-Butyl rac-(3aS,6aR)-5-[2-fluoro-4-(trifluoromethyl) phenoxy]-3,3a,4,5,6,6a-hexahydro-1H-cyclopenta[c]pyrrole-2-carboxylate

To a solution of 2-fluoro-4-(trifluoromethyl) phenol (500.0 mg, 2.78 mmol), tert-butyl 5-hydroxy-3,3a,4,5,6,6a-hexahydro-1H-cyclopenta[c]pyrrole-2-carboxylate (631.04 mg, 2.78 mmol, CAS RN 203663-25-6) and triphenylphosphine (1092.25 mg, 4.16 mmol) in toluene (15 mL) was added diethylazodicarboxylate (725.22 mg, 4.16 mmol) and the mixture was stirred at 100° C. under N₂ atmosphere for 16 h. The mixture was concentrated and purified by reverse flash (0.05% v/v FA condition) to give the desired product as off-white solid. MS (ESI): m/z=334.1 [M-C4H8+H]⁺.

Step 2: rac-(3aS,6aR)-5-[2-Fluoro-4-(trifluoromethyl) phenoxy]-1,2,3,3a,4,5,6,6a-octahydrocyclopenta[c]pyrrole 2,2,2-trifluoroacetic Acid

The product was obtained in analogy to BB14, step 2 starting from tert-butyl rac-(3aS,6aR)-5-[2-fluoro-4-(trifluoromethyl) phenoxy]-3,3a,4,5,6,6a-hexahydro-1H-cyclopenta[c]pyrrole-2-carboxylate and TFA to get the title compound as yellow oil. MS (ESI): m/z=289.9 [M+H]⁺.

### BB33

### 5-(Azetidin-3-yl)-2-(2-chlorophenoxy)pyridine 4-methylbenzenesulfonic Acid

Step 1: tert-Butyl 3-[6-(2-chlorophenoxy)-3-pyridyl]azetidine-1-carboxylate

The product was obtained in analogy to BB9, step 1 starting from tert-butyl 3-(4-bromophenyl)azetidine-1-carboxylate (CAS RN 1203681-52-0) and 5-bromo-2-(2-chlorophenoxy)pyridine (CAS N 1240670-82-9) to get the title compound as a yellow oil. MS (ESI): m/z=361.2 [M+H]⁺.

Step 2:5-(Azetidin-3-yl)-2-(2-chlorophenoxy)pyridine 4-methylbenzenesulfonic Acid

The product was obtained in analogy to BB24, step 5 starting from tert-butyl 3-[6-(2-chlorophenoxy)-3-pyridyl]azetidine-1-carboxylate to get the title compound as a colorless solid. MS (ESI): m/z=261.1 [M+H]⁺.

### BB34

### 1-[4-(Azetidin-3-yl)phenyl]-3-(2,2,2-trifluoroethoxy)azetidine 4-methylbenzenesulfonic Acid

Step 1: tert-Butyl 3-[4-[3-(2,2,2-trifluoroethoxy)azetidin-1-yl]phenyl]azetidine-1-carboxylate

To a suspension of tert-butyl 3-(4-bromophenyl)azetidine-1-carboxylate (222 mg, 711 μmol, CAS RN CAS RN 1203681-52-0) and 3-(2,2,2-trifluoroethoxy)azetidine (110 mg, 711 μmol, CAS RN 1333106-09-4) in tert.butanol (4 mL) under argon were added x-phos (30.5 mg, 64 μmol, CAS RN CAS RN 564483-18-7), tris(dibenzylideneacetone)dipalladium (0) chloroform adduct (22.1 mg, 21.3 μmol, CAS RN 052522-40-4), and cesium carbonate (927 mg, 2.84 mmol) and the mixture was heated in a microwave oven at 90° C. for 1 hour. The mixture was filtered, the filtrate was treated with silica gel and evaporated. The compound was purified by silica gel chromatography on a 12 g column using an MPLC (ISCO) system eluting with a gradient of n-heptane:EtOAc (100:0 to 50:50) to get the desired compound as a light yellow oil (0.127 g; 44.4%). MS (ESI): m/z=387.2 [M+H]⁺.

Step 2:1-[4-(Azetidin-3-yl)phenyl]-3-(2,2,2-trifluoroethoxy)azetidine 4-methylbenzenesulfonic Acid

To a solution of tert-butyl 3-[4-[3-(2,2,2-trifluoroethoxy)azetidin-1-yl]phenyl]azetidine-1-carboxylate (670 mg, 1.73 mmol) in EtOAc (5 mL) was added 4-methylbenzenesulfonic acid hydrate (363 mg, 1.91 mmol) and the mixture was stirred at reflux in a sealed tube for 2 hours. Another batch of 4-methylbenzenesulfonic acid hydrate (109 mg, 572 μmol) was added and stirring was continued for another hour at reflux. The suspension was allowed to cool down to RT, then filtered. The filter cake was washed with a small volume of EtOAc to get the desired compound as a grey solid (0.277 g; 24.6%). MS (ESI): m/z=287.2 [M+H]⁺.

### BB35

### 5-(Azetidin-3-yl)-2-[4-(trifluoromethoxy) phenoxy]pyridine 4-methylbenzenesulfonic Acid

Step 1: tert-Butyl 3-[6-[4-(trifluoromethoxy) phenoxy]-3-pyridyl]azetidine-1-1-carboxylate

The product was obtained in analogy to BB9, step 1 starting from tert-butyl 3-(4-bromophenyl)azetidine-1-carboxylate (CAS RN 1203681-52-0) and 5-bromo-2-(4-(trifluoromethoxy) phenoxy)pyridine (CAS N 909849-01-0) to get the title compound as a light yellow waxy solid. MS (ESI): m/z=411.2 [M+H]⁺.

Step 2:5-(Azetidin-3-yl)-2-[4-(trifluoromethoxy) phenoxy]pyridine 4-methylbenzenesulfonic Acid

The product was obtained in analogy to BB34, step 1 starting from tert-Butyl 3-[6-[4-(trifluoromethoxy) phenoxy]-3-pyridyl]azetidine-1-1-carboxylate to get the title compound as a colorless solid. MS (ESI): m/z=311.1 [M+H]⁺.

### BB36

### 3-(Azetidin-3-yl)-5-[1-(trifluoromethyl)cyclopropyl]-1,2,4-oxadiazole 4-methylbenzenesulfonic Acid

Step 1: tert-Butyl 3-[(Z)—N′-[1-(trifluoromethyl)cyclopropanecarbonyl]oxycarbamimidoyl]azetidine-1-carboxylate

To a solution of 1-(trifluoromethyl)cyclopropane-1-carboxylic acid (1.43 g, 9.29 mmol, CAS RN 277756-46-4), DIPEA (3.60 g, 27.87 mmol) and HATU (4.24 g, 11.15 mmol) in DCM (40 mL) was added tert-butyl 3-(N-hydroxycarbamimidoyl)azetidine-1-carboxylate (2.0 g, 9.29 mmol, CAS RN 1309207 May 3), then stirred at 20° C. for 16 h. The mixture was evaporated and purified by reverse flash (FA) to give the desired product as light brown oil (2.6 g, 79.6% yield). MS (ESI): m/z=296.3 [M-C4H8+H]⁺.

Step 2: tert-Butyl 3-[5-[1-(trifluoromethyl)cyclopropyl]-1,2,4-oxadiazol-3-yl]azetidine-1-carboxylate

To a solution of tert-butyl 3-[(Z)—N′-[1-(trifluoromethyl)cyclopropanecarbonyl]oxycarbamimidoyl]azetidine-1-carboxylate (1.5 g, 4.27 mmol) in EtOH (37.5 mL) and water (37.5 mL) was added KOAc (838.04 mg, 8.54 mmol), the mixture was stirred at 80° C. for 12 h, The mixture was concentrated and diluted with EtOAc (50 mL), washed with water and brine, dried over Na2SO4, then concentrated, the residue was purified by reverse flash (FA) to give the desired compound as light yellow oil. (1.25 g, 87.8% yield). MS (ESI): m/z=278.4 [M-C4H8+H]⁺.

Step 3:3-(Azetidin-3-yl)-5-[1-(trifluoromethyl)cyclopropyl]-1,2,4-oxadiazole 4-methylbenzenesulfonic Acid

The product was obtained in analogy to BB34, step 1 starting from tert-Butyl 3-[5-[1-(trifluoromethyl)cyclopropyl]-1,2,4-oxadiazol-3-yl]azetidine-1-carboxylate to get the title compound as a brown waxy solid. MS (ESI): m/z=234.4 [M+H]⁺.

### BB37

### 4-Methylbenzenesulfonic acid 6-[6-(trifluoromethyl)pyrazin-2-yl]oxy-2-azaspiro[3.3]heptane

Step 1: tert-Butyl 6-[6-(trifluoromethyl)pyrazin-2-yl]oxy-2-azaspiro[3.3]heptane-2-carboxylate

To a solution of tert-butyl 6-hydroxy-2-azaspiro[3.3]heptane-2-carboxylate (350 mg, 1.64 mmol, CAS RN 1147557-97-8) in DMF (5 ml) was added NaH (43.3 mg, 1.81 mmol) and the mixture was stirred for 30 minat RT followed by addition of 2-chloro-6-(trifluoromethyl)pyrazine (38.3 mg, 210 μmol, CS RN 61655-69-4). The reaction mixture was then stirred at 90° C. for 18 hours. After cooling down to RT, water (0.2 mL) was added. The mixture is transferred to a separations funnel, extracted with EtOAc (3*25 ml) water (1*25 ml) and brine (1*25 ml). The organic layers were dried over MgSO₄, filtered and evaporated. The compound was purified by silica gel chromatography on a 24 g column using an MPLC (ISCO) system eluting with a gradient of n-heptane:EtOAc (100:0 to 60:40) to get the title compound as colorless oil (304 mg, 51.65). MS (ESI): m/z=411.2 [M+H]⁺.

Step 2:4-Methylbenzenesulfonic acid 6-[6-(trifluoromethyl)pyrazin-2-yl]oxy-2-azaspiro[3.3]heptane

The product was obtained in analogy to BB34, step 1 starting from tert-butyl 6-[6-(trifluoromethyl)pyrazin-2-yl]oxy-2-azaspiro[3.3]heptane-2-carboxylate to get the title compound as a colorless solid. MS (ESI): m/z=311.1 [M+H]⁺.

### BB38

### Lithium 2-(3,3,3-trifluoropropoxy)spiro[3.3]heptane-6-carboxylate

Step 1: Methyl 2-(3,3,3-trifluoropropoxy)spiro[3.3]heptane-6-carboxylate

To a solution of 3,3,3-trifluoropropan-1-ol (1.39 mL, 15.7 mmol, CAS RN 1138480-98-4) and methyl 6-oxospiro[3.3]heptane-2-carboxylate (2.2 g, 13.08 mmol, CAS RN 1138480-98-4) in DCM (16.5 ml) were added triethylsilane (4.6 mL, 28.7 mmol) and bismuth (III) chloride (2.06 g, 6.55 mmol). Stirring was continued at RT overnight. The suspension was filtered. The filtrate was treated with silica gel and evaporated. The compound was purified by silica gel chromatography on a 24 g column using an MPLC (ISCO) system eluting with a gradient of n-heptane:EtOAc (100:0 to 75:25) to get a crude batch (570 mg). The compound was purified by silica gel chromatography on a 40 g column using an MPLC (ISCO) system eluting with a gradient of DCM:EtOAc (100:0 to 95:5) to get the desired compound as a colorless oil (0.345 mg, 13.4%). Used s is for next step.

Step 2: Lithium 2-(3,3,3-trifluoropropoxy)spiro[3.3]heptane-6-carboxylate

To a mixture of methyl 2-(3,3,3-trifluoropropoxy)spiro[3.3]heptane-6-carboxylate (222 mg, 834 μmol1) in Water (666 μL) and THF (888 μL) was added lithium hydroxide monohydrate (43.7 mg, 1.04 mmol) and the mixture was stirred in a sealed tube at RT (30° C.) for 4 hours. The clear, colorless solution was completely evaporated to get the desired compound as a colorless solid (0.206 g; 95.7%). MS (ESI): m/z=251.1 [M−H]⁻.

### BB39

### 5-(Azetidin-3-yl)-2-[3-(trifluoromethyl)pyrrolidin-1-yl]pyridine

Step 1: tert-Butyl 3-[6-[3-(trifluoromethyl)pyrrolidin-1-yl]-3-pyridyl]azetidine-1-carboxylate

The product was obtained in analogy to BB9, step 1 starting from tert-butyl 3-(4-bromophenyl)azetidine-1-carboxylate (CAS RN 1203681-52-0) and 5-bromo-2-[3-(trifluoromethyl)pyrrolidin-1-yl]pyridine (CAS RN 1852360-62-3) to get the title compound as a light yellow waxy solid. MS (ESI): m/z=372.1 [M+H]⁺.

### Step 2:5-(Azetidin-3-yl)-2-[3-(trifluoromethyl)pyrrolidin-1-yl]pyridine

To a solution of tert-Butyl 3-[6-[3-(trifluoromethyl)pyrrolidin-1-yl]-3-pyridyl]azetidine-1-carboxylate (408 mg, 1.1 mmol) in EtOAc (7 mL) tosic acid (422 mg, 1.11 mmol) was added and the mixture was stirred at reflux over night. The mixture was evaporated, the residue resolved in 3 ml MeOH and added to an amino-functionalized silica gel column (10 g). The column was washed with ACN/MeOH 1:1 as long as the filtrate is basic (approx. 50 mL). The residue is evaporated to get the title compound as a light yellow waxy solid (318 mg, 93.9%). MS (ESI): m/z=272.1 [M+H]⁺.

### BB40

### (3S)-3-[3-Chloro-5-(2,2,2-trifluoroethoxy) phenoxy]pyrrolidine 2,2,2-trifluoroacetic Acid

Step 1: tert-Butyl (3S)-3-(3-chloro-5-hydroxy-phenoxy) pyrrolidine-1-carboxylate

To a solution of 5-chlorobenzene-1,3-diol (0.38 mL, 2.42 mmol, CAS RN 52780-23-1), (R)-(−)—N—BOC-3-pyrrolidinol (408.0 mg, 2.18 mmol, CAS RN 109431-87-0) and tributylphosphine (0.9 mL, 3.63 mmol) in THF (8 mL) was added at 0° C., and then 1,1′-azodicarbonyl-dipiperidine (0.71 mL, 3.63 mmol) was added and stirred at 50° C. for 12 h. The mixture was concentrated and purified by reverse flash (0.05% v/v FA condition) to give the desired product as yellow oil (71 mg, 9.3% yield). ¹H NMR (400 MHz, CHLOROFORM-d) δ=6.58-6.40 (m, 2H), 6.29 (br d, J=19.2 Hz, 1H), 4.82 (br s, 1H), 3.69-3.40 (m, 4H), 2.27-2.06 (m, 2H), 1.51 (br s, 9H).

Step 2: tert-Butyl (3S)-3-[3-chloro-5-(2,2,2-trifluoroethoxy) phenoxy]pyrrolidine-1-carboxylate

A solution of tert-butyl (3S)-3-(3-chloro-5-hydroxy-phenoxy) pyrrolidine-1-carboxylate (70.0 mg, 0.220 mmol), 2,2,2-trifluoroethyl trifluoromethanesulfonate (62.13 mg, 0.270 mmol) and K2CO₃ (61.53 mg, 0.450 mmol) in DMF (2 mL) was stirred at 100° C. for 12 h. The mixture was diluted by H₂O (10 mL) and extracted with EtOAc (3×10 mL), the combined organic phase was washed with brine, dried over Na2SO4, concentrated to give the desired product as yellow oil (80 mg, 0.200 mmol, 90.6% yield). ¹H NMR (400 MHz, CHLOROFORM-d) δ=6.57 (br d, J=2.0 Hz, 2H), 6.38 (t, J=2.2 Hz, 1H), 4.85 (br s, 1H), 4.32 (q, J=8.0 Hz, 2H), 3.68-3.39 (m, 4H), 2.27-2.04 (m, 2H), 1.48 (s, 9H).

Step 3: (3S)-3-[3-Chloro-5-(2,2,2-trifluoroethoxy) phenoxy]pyrrolidine 2,2,2-trifluoroacetic Acid

To a solution of tert-butyl (3S)-3-[3-chloro-5-(2,2,2-trifluoroethoxy) phenoxy]pyrrolidine-1-carboxylate (80.0 mg, 0.200 mmol) in DCM (6 mL) was added trifluoroacetic acid (0.16 mL, 2.12 mmol) and the mixture was stirred at 20° C. for 2 h. The mixture was concentrated to give the desired product as yellow oil (140 mg, 98.0%). ¹H NMR (400 MHz, CHLOROFORM-d) 8-6.73-6.32 (m, 3H), 5.09 (br s, 1H), 4.41-4.27 (m, 2H), 3.62 (br s, 4H), 2.52-2.25 (m, 2H).

### BB41

### (3R)-3-[3-Chloro-5-(2,2,2-trifluoroethoxy) phenoxy]pyrrolidine 2,2,2-trifluoroacetic Acid

Step 1: tert-Butyl (3R)-3-(3-chloro-5-hydroxy-phenoxy) pyrrolidine-1-carboxylate

The product was obtained in analogy to BB40, step 1 from(S)-(−)—N—BOC-3-pyrrolidinol (408.0 mg, 2.18 mmol, CAS RN 101469-92-5) as yellow oil. MS (ESI): m/z=258.1 [M-C4H8+H]⁺.

Step 2: tert-Butyl (3R)-3-[3-chloro-5-(2,2,2-trifluoroethoxy) phenoxy]pyrrolidine-1-carboxylate

The product was obtained in analogy to BB40, step 2 from tert-butyl (3R)-3-(3-chloro-5-hydroxy-phenoxy) pyrrolidine-1-carboxylate as yellow oil. MS (ESI): m/z=340.1 [M-C4H8+H]⁺.

Step 3: (R)-3-[3-Chloro-5-(2,2,2-trifluoroethoxy) phenoxy]pyrrolidine 2,2,2-trifluoroacetic Acid

The product was obtained in analogy to BB40, step 3 from tert-butyl (3R)-3-[3-chloro-5-(2,2,2-trifluoroethoxy) phenoxy]pyrrolidine-1-carboxylate as yellow oil. MS (ESI): m/z=296.1 [M+H]⁺.

### BB42

### 8-(3-Cyclopropyl-1,2,4-oxadiazol-5-yl)-8-azabicyclo[3.2.1]octane-3-carboxylic Acid

Step 1: Methyl 8-cyano-8-azabicyclo[3.2.1]octane-3-carboxylate

To a two-layer mixture of methyl 8-azabicyclo[3.2.1]octane-3-carboxylate hydrochloride (200 mg, 972 μmol, CAS RN 179022-43-6) in DCM (4 mL) and Water (0.2 mL) was added sodium bicarbonate (245 mg, 2.92 mmol) followed by addition of cyanic bromide (124 mg, 1.17 mmol, CAS RN 506-68-3). The mixture was vigorously stirred overnight and then diluted with water and DCM and the layers were separated. The aqueous layer was extracted twice with DCM. The organic layers were washed with brine, dried and evaporated to get the desired compound as a colorless gum (0.137 g; 72.5%). MS (ESI): m/z=195.1 [M+H]⁺.

Step 2: Ethyl 8-(3-cyclopropyl-1,2,4-oxadiazol-5-yl)-8-azabicyclo[3.2.1]octane-3-carboxylate

To a solution of methyl 8-cyano-8-azabicyclo[3.2.1]octane-3-carboxylate (137 mg, 705 μmol) and (Z)—N′-hydroxycyclopropanecarboximidamide (70.6 mg, 705 μmol, CAS RN 51285-13-3) in Ethanol (3 mL) was added dropwise zinc chloride 1.9 M in 2-Me-THF (557 μL, 1.06 mmol) and the mixture was stirred at RT for 2 hours. To the colorless suspension was added hydrochloric acid conc. (704 μL, 8.46 mmol) which led to a solution. This solution was stirred overnight at 50° C. Reaction was evaporated. The residue was neutralized with saturated aqueous NaHCO₃ solution. The aqueous layer was extracted three times with EtOAc. The organic layers were dried over MgSO₄, filtered, treated with silica gel and evaporated. The compound was purified by silica gel chromatography on a 4 g column using an MPLC system eluting with a gradient of n-heptane:EtOAc (100:0 to 0:100) to get the desired compound as a colorless oil (0.065 g; 26.2%). MS (ESI): m/z=292.1 [M+H]⁺.

Step 3:8-(3-Cyclopropyl-1,2,4-oxadiazol-5-yl)-8-azabicyclo[3.2.1]octane-3-carboxylic Acid

To a solution of ethyl 8-(3-cyclopropyl-1,2,4-oxadiazol-5-yl)-8-azabicyclo[3.2.1]octane-3-carboxylate (65 mg, 0.185 mmol) in 1,4-dioxane (1 mL) and water (1 mL) was added lithium hydroxide monohydrate (9.71 mg, 0.231 mmol) and the solution was stirred at RT for 5 hours. Dioxane was removed by evaporation. To the residue was added 1 M HCl (231.46 μL, 0.231 mmol) and the mixture was stirred at RT overnight. The suspension was filtered. The filter cake was washed with water to get the desired compound as a colorless solid (0.042 g; 64.6%). MS (ESI): m/z=264.2 [M+H]⁺.

### BB43

### 2-[[1-(Difluoromethyl)cyclopropyl]methoxy]spiro[3.3]heptane-6-carboxylic Acid

Step 1: Methyl 8-cyano-8-azabicyclo[3.2.1]octane-3-carboxylate

Sodium hydride, 60% in oil (102.18 mg, 2.55 mmol) was suspended in 5 ml of DMF and then cooled to 0° C. The solution of 2-hydroxyspiro[3.3]heptane-6-carboxylic acid (190.0 mg, 1.22 mmol, CAS RN 889944-59-6) in 5 ml of DMF was added dropwise. The resulting mixture was stirred until gas evolution ceased. At the same temperature [1-(difluoromethyl)cyclopropyl]methyl 4-methylbenzenesulfonate (448.18 mg, 1.46 mmol, CAS RN 2145815-19-4) was added in 5 ml of DMF. The resulting mixture was warmed to r.t. and stirred overnight. The solution was poured into water and extracted twice with 50 ml of MTBE. Organic phases were combined, washed with water, dried over Na2SO4 and the solvent was removed under vacuum. The crude product was purified by preparative HPLC on a SunFire C18 column using a gradient of acetonitrile:water (neutral) to the desired product as a colorless solid. (16 mg, 5.05%) as white powder. MS (ESI): m/z=259 [M−H]⁻.

### BB44

### 3-[4-(4-Chloro-2-methylsulfonyl-phenyl)phenyl]azetidine 4-methylbenzenesulfonic Acid

Step 1: tert-Butyl 3-[4-(4-chloro-2-fluoro-phenyl)phenyl]azetidine-1-carboxylate

A suspension of (4-chloro-2-fluorophenyl) boronic acid (140 mg, 801 μmol, CAS RN 160591-91-3), tert-butyl 3-(4-bromophenyl)azetidine-1-carboxylate (250 mg, 801 μmol, CAS RN 1203681-52-0), potassium carbonate (553 mg, 4 mmol), Water (400 μl) and tetrakis (triphenylphosphine)palladium (0) (46.3 mg, 40 μmol) in THF (4 ml) was stirred at 80° C. for 3 hours. The reaction mixture was poured on water and EtOAc and the layers were separated. The aqueous layer was extracted twice with EtOAc. The organic layers were dried over MgSO₄, filtered, treated with silica gel and evaporated. The compound was purified by silica gel chromatography on a 12 g column using an MPLC system eluting with a gradient of n-heptane:tBME /MeOH (9/1) (100:0 to 50:50) to get the desired compound as a colorless solid (0.318 g, 98.7%). MS (ESI): m/z=306.0 [M-C4H8+H]⁺.

Step 2: tert-Butyl 3-[4-(4-chloro-2-methylsulfonyl-phenyl)phenyl]azetidine-1-carboxylate

To a solution of tert-butyl 3-[4-(4-chloro-2-fluoro-phenyl)phenyl]azetidine-1-carboxylate (310 mg, 857 μmol) in DMSO (1 mL) and DMF (2.5 mL) under N₂ was added sodium methanethiolate (63 mg, 900 μmol) and the mixture was stirred at 100° C. for 2 hours. The reaction mixture was poured on water (25 ml) and extracted with TBME (2*25 ml). The organic layers were washed once with brine (25 ml), dried over MgSO₄, filtered and evaporated. The crude is purified by silica gel chromatography on a 12 g column (heptan/ethylacetate). The intermediate product (251 mg, 75.1% yield) is dissolved in DCM (15 mL) and m-chloroperbenzoic acid (464 mg, 1.88 mmol) was added. The mixture was stirred at room temperature over night. The crude solution is absorbed with Isolute HM-N, dried and purified by silica gel chromatography on a 12 g column to get the desired compound as a colorless solid (0.142 g, 36.5%). MS (ESI): m/z=366.0 [M-C4H8+H]⁺.

Step 3:3-[4-(4-Chloro-2-methylsulfonyl-phenyl)phenyl]azetidine 4-methylbenzenesulfonic Acid

A solution of tert-butyl 3-[4-(4-chloro-2-methylsulfonyl-phenyl)phenyl]azetidine-1-carboxylate (140 mg, 332 μmol) and 4-methylbenzenesulfonic acid hydrate (64.4 mg, 338 μmol) in EtOAc (3 ml) is shaked over night at 75° C. The cold off-white suspension is filtered. The filter cake was washed with diethylether to get the desired compound as a colorless solid (125 mg; 76.3%). MS (ESI): m/z=322.1 [M+H]⁺.

### BB45

### 5-[4-(Azetidin-3-yl)phenyl]-1-(2,2-dimethylpropyl)triazole 4-methylbenzenesulfonic Acid

Step 1:5-(4-Bromophenyl)-1-(2,2-dimethylpropyl)triazole

A solution of 4′-bromoacetophenone (1940.43 mg, 9.75 mmol, CAS RN 99-90-1), 1-azido-4-nitro-benzene (1600.0 mg, 9.75 mmol, CAS RN 1516-60-5), neopentylamine (1104.64 mg, 12.67 mmol, CAS RN 5813-64-9), AcOH (175.63 mg, 2.92 mmol) and 4A MS (500.0 mg) in toluene (64 mL) was stirred at 100° C. for 16 h. The reaction mixture was filtered and the filtrate was evaporated under reduced pressure to give the residue, which was then purified with silica column chromatography (PE/EA=3/1) to give the desired compound as a yellow gum (997 mg, 34.76% yield). Proton NMR: 1H NMR (400 MHz, CDCl₃) δ=7.59 (s, 1H), 7.56 (d, J=8.3 Hz, 2H), 7.16 (d, J=8.3 Hz, 2H), 4.12 (s, 2H), 0.75 (s, 9H).

Step 2: tert-Butyl 3-[4-[3-(2,2-dimethylpropyl)triazol-4-yl]phenyl]azetidine-1-carboxylate

The product was obtained in analogy to BB9, step 1 starting from tert-butyl 3-(4-bromophenyl)azetidine-1-carboxylate (CAS RN 1203681-52-0) and 5-(4-Bromophenyl)-1-(2,2-dimethylpropyl)triazole to get the title compound as a yellow oil. MS (ESI): m/z=371.4 [M+H]⁺.

Step 3:5-[4-(Azetidin-3-yl)phenyl]-1-(2,2-dimethylpropyl)triazole 4-methylbenzenesulfonic Acid

The product was obtained in analogy to BB7, step 6 starting from tert-Butyl 3-[4-[3-(2,2-dimethylpropyl)triazol-4-yl]phenyl]azetidine-1-carboxylate to get the title compound as an off-white solid. MS (ESI): m/z=271.4 [M+H]⁺.

### BB46

### 3-(2-Chloro-4-isopropylsulfonyl-phenyl)azetidine; 4-methylbenzenesulfonic Acid

Step 1:3-(2-Chloro-4-fluoro-phenyl)azetidine-1-carboxylic Acid Tert-Butyl Ester

To a 100 mL flask equipped with a stir bar were added (IR[dF(CF3)PPY]2(DTBPY))PF6(161 mg, 0.143 mmol), tert-butyl 3-bromoazetidine-1-carboxylate (3.38 g, 14.3 mmol), 1,1,1,3,3,3-hexamethyl-2-(trimethylsilyl)trisilane (4.4 mL, 14.3 mmol), 1,1,1,3,3,3-hexamethyl-2-(trimethylsilyl)trisilane (4.4 mL, 14.3 mmol) and anhydrous sodium carbonate (3.04 g, 28.6 mmol). The vial was sealed and placed under Ar before DME (29 mL) was added. To a separate vial were added Nickel (II) chloride ethylene glycol dimethyl ether complex (15.74 mg, 0.072 mmol) and 4,4′-di-tert-butyl-2,2′-bipyridine (19.22 mg, 0.072 mmol), under Ar. This vial was sealed, purged with Ar, treated with DME (4 mL), and sonicated for 5 min, before being added to the main reaction vial. The mixture was stirred and irradiated with a 420 nm lamp for 64 h, before being filtered and evaporated. Purification by FC (SiO2; heptane/EtOAc) gave the title compound (2.02 g; 39.5%) as a light yellow oil. MS (ESI): m/z=230.1 [M-tBu+H]⁺.

Step 2:3-[2-Chloro-4-(isopropylthio)phenyl]azetidine-1-carboxylic Acid Tert-Butyl Ester

To a solution of 3-(2-chloro-4-fluoro-phenyl)azetidine-1-carboxylic acid tert-butyl ester (320 mg, 0.896 mmol) in DMSO (5 mL) was added sodium; propane-2-thiolate (87.9 mg, 0.896 mmol) and the mixture was heated to 100° C. overnight. Another batch of sodium; propane-2-thiolate (87.9 mg, 0.896 mmol) was added and stirring was continued overnight at 100° C. The reaction mixture was poured on water and EtOAc and the layers were separated. The aqueous layer was extracted with EtOAc. The organic layers were washed with water, dried over MgSO4, filtered, treated with silica and evaporated. The crude product was purified by silica gel chromatography eluting with a gradient of n-heptane:EtOAc (100:0 to 70:30) to get the desired product as a colorless gum (0.173 g; 54%). MS (ESI): m/z=286.1 [M-C4H8+H]⁺.

Step 3:3-(2-Chloro-4-isopropylsulfonyl-phenyl)azetidine-1-carboxylic Acid Tert-Butyl Ester

To a solution of 3-[2-chloro-4-(isopropylthio)phenyl]azetidine-1-carboxylic acid tert-butyl ester (173 mg, 0.481 mmol) in DCM (1.5 mL) at RT was added carefully 3-chloroperoxybenzoic acid (269 mg, 1.2 mmol) and the rapidly formed suspension was stirred at RT for 2.75 h. The suspension was filtered. The filter cake was washed with a small volume of DCM. The filtrate was washed twice with aqueous half-saturated NaHCO₃ solution. The aqueous layers were extracted twice with DCM. The organic layers were dried over MgSO₄, filtered, treated with silica gel and evaporated. The reaction mixture was poured on half-saturated aqueous NH₄Cl solution and EtOAc and the layers were separated. The aqueous layer was extracted twice with EtOAc. The organic layers were washed twice with half-saturated aqueous NH₄Cl solution, dried over MgSO₄, filtered, treated with isolute and evaporated. Purification by FC (SiO2; heptane/EtOAc) gave the title compound (0.124 g, 67%) as a colorless gum. MS (ESI): m/z=318.1 [M+H]⁺.

Step 3:3-(2-Chloro-4-isopropylsulfonyl-phenyl)azetidine; 4-methylbenzenesulfonic Acid

To a solution of 3-(2-chloro-4-isopropylsulfonyl-phenyl)azetidine-1-carboxylic acid tert-butyl ester (124 mg, 0.322 mmol) in EtOAc (1.1 mL) was added 4-methylbenzenesulfonic acid acid monohydrate (67.31 mg, 0.354 mmol) and the solution was stirred at reflux in a sealed tube for 1 h. After cooling down, the suspension was filtered. The filter cake was washed with a small volume of EtOAc to get the desired product as a colorless solid (0.131 g, 87%). MS (ESI): m/z=274.2 [M+H]⁺.

### BB47

### 3-(4-tert-Butylsulfonyl-2-chloro-phenyl)azetidine; 4-methylbenzenesulfonic Acid

Step 1: tert-Butyl 3-(4-tert-butylsulfanyl-2-chloro-phenyl)azetidine-1-carboxylate

To a solution of 3-(2-chloro-4-fluoro-phenyl)azetidine-1-carboxylic acid tert-butyl ester (0.200 g, 0.560 mmol, BB46, step 1) in DMSO (2.5 mL) was added sodium; 2-methylpropane-2-thiolate (62.81 mg, 0.560 mmol) and the mixture was heated at 100° C. for two nights. Another batch of sodium; 2-methylpropane-2-thiolate (62.81 mg, 0.560 mmol) was added and stirring was continued overnight at 100° C. The reaction mixture was poured on water and EtOAc and the layers were separated. The aqueous layer was extracted twice with EtOAc. The organic layers were washed with water, dried over MgSO₄, filtered, treated with silica gel and evaporated. The crude product was purified by silica gel chromatography on a 40 g column using an MPLC (ISCO) system eluting with a gradient of n-heptane:EtOAc (100:0 to 70:30) to get the desired product as a colorless gum (0.069 g; 31.1%). MS (ESI): m/z=300.1 [M-C4H8+H]⁺.

Step 2: tert-Butyl 3-(4-tert-butylsulfonyl-2-chloro-phenyl)azetidine-1-carboxylate

To a solution of 3-[4-(tert-butylthio)-2-chloro-phenyl]azetidine-1-carboxylic acid tert-butyl ester (76.67 mg, 0.194 mmol) in DCM (0.575 mL) at ice-bath temperature was added 3-chloroperoxybenzoic acid (108.62 mg, 0.485 mmol) and the suspension was stirred at RT for 2.75 hours. The suspension was filtered. The filter cake was washed with a small volume of DCM. The filtrate was washed twice with aqueous half-saturated NaHCO₃ solution. The aqueous layers were extracted twice with DCM. The organic layers were dried over MgSO₄, filtered, treated with silica gel and evaporated. The reaction mixture was poured on half-saturated aqueous NH₄Cl solution and EtOAc and the layers were separated. The aqueous layer was extracted twice with EtOAc. The organic layers were washed twice with half-saturated aqueous NH4Cl solution, dried over MgSO₄, filtered, treated with isolute and evaporated. The crude product was purified by silica gel chromatography on a 4 g column using an MPLC (ISCO) system eluting with a gradient of n-heptane:EtOAc (100:0 to 50:50) to get the desired product as a colorless solid (0.039 g; 47.1%). MS (ESI): m/z=332.1 [M+H]⁺.

Step 3:3-(4-tert-Butylsulfonyl-2-chloro-phenyl)azetidine; 4-methylbenzenesulfonic Acid

To a solution of 3-(4-tert-butylsulfonyl-2-chloro-phenyl)azetidine-1-carboxylic acid tert-butyl ester (39 mg, 0.091 mmol) in EtOAc (0.400 mL) was added tosylic acid monohydrate (19.14 mg, 0.101 mmol) and the solution was stirred at reflux in a sealed tube for 1 hours. After cooling down, the suspension was filtered. The filter cake was washed with a small volume of EtOAc to get the desired product as a colorless solid (0.0.36 g; 83.8%). MS (ESI): m/z=288.2 [M+H]⁺.

### BB48

### 3-(2-Chloro-4-isobutylsulfonyl-phenyl)azetidine; 4-methylbenzenesulfonic Acid

Step 1:1-Bromo-2-chloro-4-isobutylsulfonyl-benzene

To an ice-cold solution of 4-bromo-3-chloro-benzenesulfonyl chloride (500 mg, 1.72 mmol, CAS RN 874801-46-4) in THF (4.5 mL) was added hydrazine; hydrate (251.42 μL, 5.17 mmol) and the solution was stirred at RT for 2 hours. THF was evaporated and the residue was triturated with heptane (5.5 mL). The slurry was decanted and the residue was diluted in ethanol (10 mL). To the suspension were added sodium acetate anhydrous (848.7 mg, 10.35 mmol) and 1-iodo-2-methylpropane (1. mL, 8.62 mmol) and the mixture was stirred at reflux overnight. After cooling down, the reaction mixture was evaporated. The residue was partioned between water and EtOAc and the layers were separated. The aqueous layer was extracted twice with EtOAc. The organic layers were dried over MgSO₄, filtered, treated with silica gel and evaporated. The crude product was purified by silica gel chromatography on a 24 g column using an MPLC (ISCO) system eluting with a gradient of n-heptane:EtOAc (100:0 to 65:35) to get the desired product as a colorless solid (0.091 g; 16.1%). MS (ESI): m/z=313.0 [M+H]⁺.

Step 2: tert-Butyl 3-(2-chloro-4-isobutylsulfonyl-phenyl)azetidine-1-carboxylate

The product was obtained in analogy to BB9, step 1 starting from 1-bromo-2-chloro-4-isobutylsulfonyl-benzene to get the title compound as a colorless oil. MS (ESI): m/z=332.1 [M-C4H8+H]⁺.

Step 3:3-(2-Chloro-4-isobutylsulfonyl-phenyl)azetidine; 4-methylbenzenesulfonic Acid

To a solution of 3-(2-chloro-4-isobutylsulfonyl-phenyl)azetidine-1-carboxylic acid tert-butyl ester (25 mg, 0.039 mmol) in EtOAc (0.300 mL) was added tosylic acid monohydrate (11.03 mg, 0.058 mmol) and the mixture was stirred at reflux for 1.5 hours. The suspension was evaporated. Used as is for next step. MS (ESI): m/z=288.2 [M+H]⁺.

### BB49

### 3-[4-(Benzenesulfonyl)phenyl]azetidine; 4-methylbenzenesulfonic acid

Step 1: tert-Butyl 3-[4-(benzenesulfonyl)phenyl]azetidine-1-carboxylate

The product was obtained in analogy to BB9, step 1 starting from 1-bromo-2-chloro-4-isobutylsulfonyl-benzene to get the title compound as a colorless solid. MS (ESI): m/z=318.1 [M-C4H8+H]⁺.

Step 2:3-[4-(Benzenesulfonyl)phenyl]azetidine; 4-methylbenzenesulfonic Acid

To a suspension of tert-butyl 3-[4-(benzenesulfonyl)phenyl]azetidine-1-carboxylate (110 mg, 0.265 mmol) in EtOAc (1 mL) was added p-toluenesulfonic acid monohydrate (63 mg, 0.331 mmol) and the suspension was stirred at reflux for 30 minutes. After cooling down, the suspension was filtered. The filter cake was washed with a small volume of EtOAc to get the desired product as a colorless solid (0.110 g; 85.6%). MS (ESI): m/z=274.1 [M+H]⁺.

### BB50

### 3-[4-(2-Chloro-4-methylsulfonyl-phenyl)phenyl]azetidine; 4-methylbenzenesulfonic Acid

Step 1: tert-Butyl 3-[4-(2-chloro-4-methylsulfonyl-phenyl)phenyl]azetidine-1-carboxylate

A suspension of 2-(2-chloro-4-mesyl-phenyl)-4,4,5,5-tetramethyl-1,3,2-dioxaborolane (304.24 mg, 0.961 mmol, CAS RN 2377012-74-1), 3-(4-bromophenyl)azetidine-1-carboxylic acid tert-butyl ester (300 mg, 0.961 mmol), tetrakis(triphenylphosphine)palladium (0) (5.55 mg, 0.005 mmol) and K₂CO₃ (664.05 mg, 4.8 mmol) in THF (3.75 mL) and water (0.375 mL) under argon was heated in the microwave oven at 110° C. for 30 minutes. The reaction mixture was poured on water and EtOAc and the layers were separated. The aqueous layer was extracted twice with EtOAc. The organic layers were dried over MgSO4, filtered, treated with silica gel and evaporated. The crude product was purified by silica gel chromatography on a 25 g column using an MPLC (ISCO) system eluting with a gradient of n-heptane:EtOAc (100:0 to 50:50) to get the desired product as a light brown oil (0.346 g; 76.8%). MS (ESI): m/z=366.1 [M-C4H8+H]⁺.

Step 2:3-[4-(2-Chloro-4-methylsulfonyl-phenyl)phenyl]azetidine; 4-methylbenzenesulfonic Acid

To a solution of 3-[4-(2-chloro-4-mesyl-phenyl)phenyl]azetidine-1-carboxylic acid tert-butyl ester (384 mg, 0.82 mmol, 1 eq) in EtOAc (2.5 mL) was added p-toluenesulfonic acid monohydrate (195 mg, 1.03 mmol, 1.25 eq) and the suspension was stirred at reflux for 30 minutes. After cooling down, the suspension was filtered. The filter cake was washed with a small volume of EtOAc to get the desired product as a colorless solid (0.300 g; 69.6%). MS (ESI): m/z=322.1 [M+H]⁺.

### BB51

### 3-(4-tert-Butylphenyl)azetidine; 4-methylbenzenesulfonic Acid

Step 1: tert-Butyl 3-(4-tert-butylphenyl)azetidine-1-carboxylate

To a stirred suspension of (4-(tert-butyl)phenyl) boronic acid (1.26 g, 7.06 mmol) in 2-Propanol (12.5 ml) was added tert-butyl 3-iodoazetidine-1-carboxylate (1 g, 3.53 mmol) at room temperature, to give a solution. To the mixture was added rac-(1R,2R)-2-aminocyclohexan-1-ol (24.4 mg, 212 μmol), nickel (II) iodide (66.2 mg, 212 μmol) and sodium bis(trimethylsilyl)amide (3.53 mL, 7.06 mmol) under argon. The mixture was heated in a microwave oven for 30 minutes at 80° C. The reaction mixture was poured on water and EtOAc and the layers were separated. The aqueous layer was extracted twice with EtOAc. The organic layers were dried over MgSO₄, filtered, treated with silica gel and evaporated. The compound was purified by silica gel chromatography on a 40 g column using an MPLC system eluting with a gradient of n-heptane:EtOAc (100:0 to 50:50) to get the desired compound as a colorless oil (0.339 g; 31.5%). MS (ESI): m/z=234.2 [M-C4H8+H]⁺.

Step 2:3-(4-tert-Butylphenyl)azetidine; 4-methylbenzenesulfonic Acid

A suspension of tert-butyl 3-(4-(tert-butyl)phenyl)azetidine-1-carboxylate (666 mg, 2.3 mmol) and 4-methylbenzenesulfonic acid monohydrate (476 mg, 2.76 mmol) in EtOAc (7 mL) was stirred at reflux for 1 hour. The suspension was allowed to cool down to RT, cooled in an ice-bath and then filtered. The filter cake was washed with cold EtOAc to get the desired compound as a colorless solid (0.636 g; 76.5%). MS (ESI): m/z=190.2 [M+H]⁺.

### BB52

### 4-(Azetidin-3-yl)-N-methyl-N-phenyl-aniline; 4-methylbenzenesulfonic Acid

Step 1:(4-Bromophenyl)-methyl-phenyl-amine

To an ice-cold solution of (4-bromophenyl)-phenyl-amine (1000 mg, 4.03 mmol, CAS RN 54446-36-5) in THF (10 mL) was added NaH (55% in mineral oil) (211.04 mg, 4.84 mmol) and the mixture was stirred at ice-bath temperature for 45 minutes. After addition of iodomethane (352.81 μL, 5.64 mmol), the mixture was stirred at RT for 19.5 hours. The reaction mixture was poured on half-saturated aqueous NH₄Cl solution and EtOAc and the layers were separated. The aqueous layer was extracted twice with EtOAc. The organic layers were dried over MgSO₄, filtered, treated with silica gel and evaporated. The crude product was purified by silica gel chromatography on a 40 g column using an MPLC (ISCO) system eluting with a gradient of n-heptane:EtOAc (100:0 to 90:10) to get the desired product as a colorless oil (0.912 g; 86.3%). MS (ESI): m/z=262.0 [M+H]⁺.

Step 2: tert-Butyl 3-[4-(N-methylanilino)phenyl]azetidine-1-carboxylate

The product was obtained in analogy to BB9, step 1 starting from (4-bromophenyl)-methyl-phenyl-amine to get the title compound as a light yellow oil. MS (ESI): m/z=339.2 [M+H]⁺.

Step 3:4-(Azetidin-3-yl)-N-methyl-N-phenyl-aniline; 4-methylbenzenesulfonic Acid

To a solution of 3-[4-(N-methylanilino)phenyl]azetidine-1-carboxylic acid tert-butyl ester (56 mg, 0.132 mmol) in EtOAc (0.400 mL) was added p-toluenesulfonic acid monohydrate (55.4 mg, 0.291 mmol) and the brown solution was stirred at reflux for 45 minutes. The mixture was allowed to cool down to RT. The resulting brown two-layer mixture was evaporated to give the desired compound as a brown foam (0.090 g; 93.3%). MS (ESI): m/z=239.1 [M+H]⁺.

### BB53

### 3-[4-(Trifluoromethylsulfonyl)phenyl]azetidine; 4-methylbenzenesulfonic Acid

Step 2: tert-Butyl 3-[4-(trifluoromethylsulfonyl)phenyl]azetidine-1-carboxylate

The product was obtained in analogy to BB9, step 1 starting from 1-bromo-4-[(trifluoromethyl) sulfonyl]benzene (CAS RN 312-20-9) to get the title compound as a light brown oil. MS (ESI): m/z=310.1 [M-C4H8+H]⁺.

Step 3:3-[4-(Trifluoromethylsulfonyl)phenyl]azetidine; 4-methylbenzenesulfonic Acid

A solution of tert-butyl 3-[4-(trifluoromethylsulfonyl)phenyl]azetidine-1-carboxylate (104 mg, 0.270 mmol) and p-toluenesulfonic acid monohydrate (61.73 mg, 0.324 mmol) in EtOAc (0.600 mL) was stirred at reflux for 1 hours. After cooling down, the suspension was filtered. The filter cake was washed with a small volume of EtOAc to get the desired compound as a light brown solid (0.101 g; 85.3%). MS (ESI): m/z=266.1 [M+H]⁺.

### BB54

### 4-(Azetidin-3-yl)-N-(cyclopropylmethyl)-N-phenyl-aniline

Step 1:4-Bromo-N-(cyclopropylmethyl)-N-phenyl-aniline

To a solution of (4-bromophenyl)-phenyl-amine (200 mg, 0.81 mmol, CAS RN 54446-36-5) in THF (2.5 mL) were added dibutyltin dichloride (49.0 mg, 0.16 mmol) and cyclopropanecarboxaldehyde (303.8 μL, 4.0 mmol) and the reaction mixture was stirred at RT for 20 min. Phenylsilane (119.2 μL, 0.97 mmol) was added to the reaction mixture and stirring at RT continued overnight. The reaction mixture was heated to 50° C. and stirring continued overnight. The crude product was purified by silica gel chromatography using a MPLC system eluting with a gradient of n-heptane:EtOAc (100:0 to 65:35). After evaporation of the solvent, the title compound was obtained as a colorless liquid (170 mg, 68%). MS (ESI): m/z=304.1 [M+H]⁺.

Step 2: tert-Butyl 3-[4-[N-(cyclopropylmethyl)anilino]phenyl]azetidine-1-carboxylate

The title compound was obtained in analogy to BB9, Step 1 starting from 4-bromo-N-(cyclopropylmethyl)-N-phenyl-aniline (165 mg, 0.55 mmol) and tert-butyl 3-bromoazetidine-1-carboxylate (193.4 mg, 0.82 mmol, 1.5 equiv; CAS RN 1064194-10-0) as a colorless oil (0.12 g, 56%). MS (ESI): m/z=379.3 [M+H]⁺.

Step 3:4-(Azetidin-3-yl)-N-(cyclopropylmethyl)-N-phenyl-aniline

A mixture of tert-butyl 3-[4-[N-(cyclopropylmethyl)anilino]phenyl]azetidine-1-carboxylate (116 mg, 0.31 mmol, 1.0 equiv) and p-toluenesulfonic acid monohydrate (128.3 mg, 0.67 mmol, 2.2 equiv) in EtOAc (1.2 mL) was heated at reflux for 1 h and stirring continued at RT for 6 h. The solvent was removed by evaporation and the crude product purified by silica gel chromatography (aminophase Si—NH2) using a MPLC system eluting with an isocratic mixture of MeOH:ACN (1:4). After evaporation of the solvent, the title compound was obtained as a light yellow oil (44 mg, 51%). MS (ESI): m/z=279.3 [M+H]⁺.

### BB55

### 5-(Azetidin-3-yl)-2-tert-butyl-pyridine

Step 1: tert-Butyl 3-(6-tert-butyl-3-pyridyl)azetidine-1-carboxylate

The title compound was obtained in analogy to BB9, Step 1 starting from 5-bromo-2-tert-butyl-pyridine (CAS RN 39919-58-9) as a light brown oil (0.12 g, 56%). MS (ESI): m/z=291.3 [M+H]⁺.

Step 2:5-(Azetidin-3-yl)-2-tert-butyl-pyridine

A solution of tert-butyl 3-(6-tert-butyl-3-pyridyl)azetidine-1-carboxylate (395 mg, 1.36 mmol) and p-toluenesulfonic acid monohydrate (517.47 mg, 2.72 mmol) in EtOAc (4.4 mL) was stirred at reflux for 1 hour. The yellow two-layer solution was allowed to cool down to RT and then evaporated. The residue was dissolved in a small volume of MeCN/MeOH 4/1. The solution was applied to a 12 g Si—NH2 column and the product was eluted with a mixture of MeCN/MeOH 1/1. ibenzylideneacetaonhe product containing fraction was evaporated to get the desired compound as a light yellow oil (0.264 g; 96.9%). MS (ESI): m/z=191.2 [M+H]⁺.

### BB56

### N-[5-(Azetidin-3-yl)-2-pyridyl]-3-ethyl-N-methyl-pyridin-2-amine

Step 1: N-(5-Bromo-2-pyridyl)-3-ethyl-pyridin-2-amine

In a 25 mL round-bottomed flask, 2,5-dibromopyridine (500 mg, 2.11 mmol) and 3-ethylpyridin-2-amine (258 mg, 2.11 mmol) were combined with Toluene (15.0 mL) to give a light brown solution. Tris(dibenzylideneacetone)dipalladium (0) (38.7 mg, 42.2 μmol) and 1,3-bis(diphenylphosphino)propane (34.8 mg, 84.4 μmol) and sodium tert-butoxide (284 mg, 2.95 mmol) were added. The reaction mixture was heated to 70° C. and stirred for 15 h. The reaction mixture was poured into 25 mL aqueous saturated NaCl solution and extracted with EtOAc (3×50 mL). The organic layers were dried over MgSO₄ and concentrated in vacuo. The crude material was purified by flash chromatography (silica gel, 40 g, 10% to 100% EtOAc in heptane). The fractions were concentrated in vacuo. The crude material was triturated with diethyl ether (5 mL). The solid was filtered through sintered glass to get the desired product as an off-white solid (0.38 g, 64.7%). MS (ESI): m/z=278.2 [M+H]⁺.

Step 2: N-(5-Bromo-2-pyridyl)-3-ethyl-N-methyl-pyridin-2-amine

To an ice-cold solution of N-(5-bromo-2-pyridyl)-3-ethyl-pyridin-2-amine (230 mg, 0.827 mmol) in THF (2 mL) was added NaH (55% in mineral oil) (43.3 mg, 0.992 mmol) and the mixture was stirred at ice-bath temperature for 45 minutes. After addition of iodomethane (72.39 uL, 1.16 mmol), the mixture was stirred at RT for 46 hours. The reaction mixture was poured on water and EtOAc and the layers were separated. The aqueous layer was extracted with EtOAc. The organic layers were washed with water, dried over MgSO₄, filtered, treated with silica gel and evaporated. The crude product was purified by silica gel chromatography on a 12 g column using an MPLC (ISCO) system eluting with a gradient of n-heptane:EtOAc (100:0 to 50:50) to get the desired product as a light yellow oil (0.117 g; 46.5%). MS (ESI): m/z=292.1 [M+H]⁺.

Step 3: tert-Butyl 3-[6-[(3-ethyl-2-pyridyl)-methyl-amino]-3-pyridyl]azetidine-1-carboxylate

The title compound was obtained in analogy to BB9, Step 1 starting from N-(5-bromo-2-pyridyl)-3-ethyl-N-methyl-pyridin-2-amine as a light yellow oil. MS (ESI): m/z=369.3 [M+H]⁺.

Step 2: N-[5-(Azetidin-3-yl)-2-pyridyl]-3-ethyl-N-methyl-pyridin-2-amine

A solution of tert-butyl 3-[6-[(3-ethyl-2-pyridyl)-methyl-amino]-3-pyridyl]azetidine-1-carboxylate (98 mg, 0.266 mmol) and p-toluenesulfonic acid monohydrate (202.37 mg, 1.06 mmol) in EtOAc (1 mL) was heated at reflux for 1 hour. The rapidly formed two-layer solution was evaporated and the residue dissolved in a small volume of MeCN/MeOH 4/1. It was applied on a 12 g Si—NH2 column and eluted with MeCN/MeOH 9/1 to get the desired product as a light yellow oil (0.055 g, 77.1%). MS (ESI): m/z=269.3 [M+H]⁺.

### BB57

### 3-[4-(2,2,2-Trifluoro-1,1-dimethyl-ethoxy)phenyl]azetidine; 4-methylbenzenesulfonic Acid

Step 1:1-Bromo-4-((1,1,1-trifluoro-2-methylpropan-2-yl)oxy)benzene

To a solution of 4-((1,1,1-trifluoro-2-methylpropan-2-yl)oxy)aniline (3.00 g, 13.7 mmol, CAS RN 1255649-29-6) in Acetonitrile (66 mL) was added copper (II) bromide (3.36 g, 15.1 mmol). To the dark suspension was added dropwise a solution of tert-butyl nitrite (1.99 mL, 15.1 mmol) in Acetonitrile (16.66 mL) over 30 minutes and the mixture was stirred at RT overnight. It was diluted with Acetonitrile, treated with silica gel and evaporated. The compound was twice purified by silica gel chromatography on a 120 g column using an MPLC system eluting with n-heptane as an isocrate to get the desired compound as a colorless oil (1.7 g; 35.5%).

Step 2: tert-Butyl 3-[4-(2,2,2-trifluoro-1,1-dimethyl-ethoxy)phenyl]azetidine-1-carboxylate

The title compound was obtained in analogy to BB9, Step 1 starting from 1-bromo-4-((1,1,1-trifluoro-2-methylpropan-2-yl)oxy)benzene as a light brown solid. MS (ESI): m/z=304.1 [M-C4H8+H]⁺.

Step 3:3-[4-(2,2,2-Trifluoro-1,1-dimethyl-ethoxy)phenyl]azetidine; 4-methylbenzenesulfonic Acid

A solution of tert-butyl 3-(4-((1,1,1-trifluoro-2-methylpropan-2-yl)oxy)phenyl)azetidine-1-carboxylate (1.14 g, 3.17 mmol) and 4-methylbenzenesulfonic acid hydrate (634 mg, 3.33 mmol, Eq) in EtOAc (7.6 ml) was stirred at reflux for 1.5 hours. The rapidly formed suspension was stored in the fridge for 5 days, then filtered. The filter cake was washed with a small volume of ethyl acetate to get the desired compound as a colorless solid (0.905 g; 59.9%). MS (ESI): m/z=260.2 [M+H]⁺.

### BB58

### 1-[4-(Azetidin-3-yl)phenyl]-3-(2,2,2-trifluoroethoxy)azetidine; 4-methylbenzenesulfonic acid

Step 1: tert-Butyl 3-[4-[3-(2.2.2-trifhioroethoxy)azetidin-1-yl]phenyl]azetidine-1-carboxylate

To a suspension of tert-butyl 3-(4-bromophenyl)azetidine-1-carboxylate (115 mg, 0.37 mmol, 1.0 equiv; BB 6/Step 1; CAS RN 1203681-52-0) and 3-(2,2,2-trifluoroethoxy)azetidine (57.1 mg, 0.37 mmol, 1.0 equiv; CAS RN 1333106-09-4) in tert-butanol (2.2 mL) under Ar were added X-PHOS (15.8 mg, 33.2 μmol, 0.09 equiv; CAS RN 564483-18-7), tris (dibenzylideneacetone)dipalladium (0) chloroform adduct (11.4 mg, 11.1 μmol, 0.03 equiv; CAS RN 52522-40-4) and cesium carbonate (480 mg, 1.47 mmol, 4.0 equiv) and the reaction mixture was heated by microwave irradiation to 90° C. for 30 min. The reaction mixture was filtered, the filtrate treated with silica gel and concentrated. The compound was purified by silica gel chromatography using a MPLC system eluting with a gradient of n-heptane:EtOAc (100:0 to 50:50) to get the title compound as a light yellow oil (74 mg, 48%). MS (ESI): m/z=387.2 [M+H]⁺.

Step 2:1-[4-(Azetidin-3-ylphenyl]-3-(2.2.2-trifluoroethoxy)azetidine; 4-methylbenzenesulfonic Acid

To a solution of tert-butyl 3-[4-[3-(2,2,2-trifluoroethoxy)azetidin-1-yl]phenyl]azetidine-1-carboxylate (0.67 g, 1.73 mmol, 1.0 equiv) in EtOAc (5 mL) was added 4-methylbenzenesulfonic acid hydrate (0.47 g, 2.48 mmol, 1.4 equiv) and the reaction mixture was heated at reflux overnight. The reaction mixture was cooled to RT, filtered and the precipitate washed with EtOAc to afford the title compound as a grey solid (0.28 g, 25%). MS (ESI): m/z=287.2 [M+H]⁺.

### BB60

### [3-[4-(4-Chloro-2-methylsulfonyl-phenyl)phenyl]azetidin-1-yl]-(3-hydroxypyrrolidin-1-yl)methanone

A solution of 3-[4-(4-chloro-2-methylsulfonyl-phenyl)phenyl]azetidine; 4-methylbenzenesulfonic acid (70 mg, 0.139 mmol, BB44), 1,1′-Carbonyl-di-(1,2,4-triazole) (27.34 mg, 0.167 mmol) and DIEA (169.76 μL, 0.972 mmol) was stirred at RT for 2 hours. The clear solution was added to DL-3-pyrrolidinol (13.5 μL, 0.167 mmol, CAS RN 40499-83-0) and stirring was continued at 50° C. overnight. The solution was evaporated. The product was purified on a preparative HPLC (YMC-Triart C18) using a gradient acetonitrile:water+0.1% HCOOH. The product containing fractions were evaporated to get the desired compound as a white crystalline solid (0.022 g; 37.2%). MS (ESI): m/z=435.1 [M+H]⁺.

### BB61

### [3-[4-(4-Chloro-2-methylsulfonyl-phenyl)phenyl]azetidin-1-yl]-[3-(hydroxymethyl)pyrrolidin-1-yl]methanone

A solution of 3-[4-(4-chloro-2-mesyl-phenyl)phenyl]azetidine; tosylic acid (70.03 mg, 0.139 mmol, BB44), 1′-Carbonyl-di-(1,2,4-triazole) (22.8 mg, 0.139 mmol) and DIEA (117.86 μL, 0.675 mmol) was stirred at RT for 3 hours. The clear solution was added to pyrrolidin-3-ylMeOH (11.96 μL, 0.116 mmol, CAS RN 5082-74-6) and stirring was continued at 50° C. overnight. The solution was evaporated. The product was purified on a preparative HPLC (YMC-Triart C18) using a gradient acetonitrile:water+0.1% HCOOH. The product containing fractions were evaporated to get the desired compound as a colourless gum (0.018 g; 33.8%). MS (ESI): m/z=449.2 [M+H]⁺.

### BB62

### 2-(Azetidin-3-yl)-5-(2-chloro-4-methylsulfonyl-phenyl)pyridine; 4-methylbenzenesulfonic Acid

Step 1: tert-Butyl 3-(5-bromo-2-pyridyl)azetidine-1-carboxylate

To a suspension of zinc dust (346.4 mg, 5.3 mmol) in THF (8 mL) were added 1,2-dibromoethane (30.44 μL, 0.353 mmol) and chlorotrimethylsilane (44.93 μL, 0.353 mmol) and the suspension was stirred at 60° C. for 15 min. A solution of tert-butyl 3-iodoazetidine-1-carboxylate (613.5 μL, 3.53 mmol, CAS RN 254454-54-1) in N,N-dimethylacetamide (8 mL) was added. The reaction mixture was stirred an additional 15 min at 60° C. before cooling to rt. 5-bromo-2-iodopyridine (1.05 g, 3.71 mmol, CAS RN 223463-13-6), 1,1′-bis(diphenylphosphino) ferrocene-palladium(II) dichloride DCM complex (144.23 mg, 0.177 mmol, CAS RN 95464 May 4) and cuprous iodide (34.32 mg, 0.177 mmol) were added and stirring was continued at 80° C. for 2 hours. The reaction mixture was diluted with EtOAc and water and the mixture was filtered. The filtrate layers were separated. The aqueous layer was extracted twice with EtOAc. The organic layers were washed twice with water, dried over MgSO4, filtered, treated with silica gel and evaporated. The product was purified by silica gel chromatography on a 25 g column using an MPLC (ISCO) system eluting with a gradient of n-heptane:EtOAc (100:0 to 50:50) to get the desired compound as a light brown oil (0.713 g; 61.2%). MS (ESI): m/z=257.0 [M-C4H8+H]⁺.

Step 2: tert-Butyl 3-[5-(2-chloro-4-methylsulfonyl-phenyl)-2-pyridyl]azetidine-1-carboxylate

To a solution of tert-butyl 3-(5-bromo-2-pyridyl)azetidine-1-carboxylate (100 mg, 0.303 mmol) in THF (1.5 mL) and water (0.150 mL) under argon were added 2-(2-chloro-4-mesyl-phenyl)-4,4,5,5-tetramethyl-1,3,2-dioxaborolane (115.24 mg, 0.364 mmol, CAS RN 2377012-74-1), potassium carbonate (209.62 mg, 1.52 mmol) and tetrakis(triphenylphosphine)palladium (0) (17.53 mg, 0.015 mmol) and the mixture was heated in a sealed tube for 2.5 hours at 80° C. After cooling down, the reaction mixture was poured on water and EtOAc and the layers were separated. The aqueous layer was extracted twice with EtOAc. The organic layers were combined, dried over MgSO₄, filtered, treated with silica gel and evaporated. The product was purified by silica gel chromatography on a 4 g column using an MPLC (ISCO) system eluting with a gradient of n-heptane:EtOAc (100:0 to 0:100) to get the desired compound as a light brown gum (0.127 g; 94.0%). MS (ESI): m/z=367.0 [M-C4H8+H]⁺.

Step 3:2-(Azetidin-3-yl)-5-(2-chloro-4-methylsulfonyl-phenyl)pyridine; 4-methylbenzenesulfonic Acid

A solution of 3-[5-(2-chloro-4-mesyl-phenyl)-2-pyridyl]azetidine-1-carboxylic acid tert-butyl ester (127 mg, 0.285 mmol) and p-toluenesulfonic acid monohydrate (119.38 mg, 0.628 mmol) in EtOAc (1 mL) was stirred at 77° C. for 1 hour. The mixture was cooled to give a slurry. Addition of some MeOH led to a solution which was slowly evaporated to give a suspension, which was filtered. The filter cake was washed with a small volume of EtOAc to get the desired product as an off-white solid (0.160 g, 84.0%). MS (ESI): m/z=323.0 [M+H]⁺.

### BB 63

### 2-(Azetidin-3-yl)-5-(4-chloro-2-methylsulfonyl-phenyl)pyridine; 4-methylbenzenesulfonic Acid

Step 1: tert-Butyl 3-[5-(4-chloro-2-methylsulfonyl-phenyl)-2-pyridyl]azetidine-1-carboxylate

The title compound was obtained in analogy to BB 62, step 2 starting from tert-butyl 3-(5-bromo-2-pyridyl)azetidine-1-carboxylate (227.5 mg, 0.69 mmol, BB62, step 1) and 2-(4-chloro-2-methylsulfonyl-phenyl)-4,4,5,5-tetramethyl-1,3,2-dioxaborolane (230 mg, 0.69 mmol, CAS RN 13136117-75-2) as a colorless foam (221 mg, 76%). MS (ESI): m/z=367.0 [M+2H-tBu]⁺.

Step 2:2-(Azetidin-3-yl)-5-(4-chloro-2-methylsulfonyl-phenyl)pyridine; 4-methylbenzenesulfonic Acid

To a solution of 3-[5-(4-chloro-2-mesyl-phenyl)-2-pyridyl]azetidine-1-carboxylic acid tert-butyl ester (60 mg, 0.142 mmol) in EtOAc (0.4 mL) was added p-toluenesulfonic acid monohydrate (59.37 mg, 0.312 mmol) and the mixture was stirred at reflux for 1 hours. The slurry was evaporated to get the desired product as a light brown foam (0.094 g, 99.3%). MS (ESI): m/z=323.1 [M+H]⁺.

### BB65

### 2-[4-(Azetidin-3-yl)phenyl]-5-chloro-3-methylsulfonyl-pyridine; 4-methylbenzenesulfonic Acid

Step 1:2-Bromo-5-chloro-3-methylsulfonyl-pyridine

Mixture A: To ice-cold water (2.61 mL, 144.61 mmol) in a three neck flask was added dropwise below −5° C. thionyl chloride (439.8 μL, 6.03 mmol) and the solution was allowed to warm up overnight before adding copper(I) chloride (4.8 mg, 0.048 mmol). Mixture B: To ice cold 2-bromo-5-chloro-pyridin-3-amine (500 mg, 2.41 mmol) was added portionwise 12 M HCl (2.41 mL, 28.92 mmol) and the suspension was allowed to stir at RT for 15 min before again cooling down to −8° C. A solution of sodium nitrite (199.6 mg, 2.89 mmol) in water (0.8 mL) was added dropwise over 20 min between −8° C. and −10° C. The reaction mixture was stirred at approx. −10° C. for another 30 min. Mixture B was portionwise transferred to Mixture A between −8° C. and −10° C. over 10 min. The reaction mixture was allowed to stir at 0° C. for 1.5 h. TBME was added to the suspension and the layers were separated. The aqueous layer was washed twice with TBME. The organic layers were washed with a sat. aqueous NaCl solution, dried over MgSO₄, filtered and evaporated. The crude product was dissolved in THF (2.5 mL) and added to a stirred solution of sodium bicarbonate (136.7 mg, 1.63 mmol, 0.68 equiv) and sodium sulfite (188.3 mg, 1.49 mmol, 0.62 equiv) in water (4 mL) and the reaction mixture was vigorously stirred at 75° C. for 2 h. After cooling down to RT, iodomethane (752.6 mg, 331.5 μL, 5.3 mmol, 2.2 equiv) was added and stirring was continued overnight at 50° C. After cooling down the reaction mixture was diluted with EtOAc and water and the layers were separated. The aqueous layer was extracted twice with EtOAc. The organic layers were washed once with water, dried over MgSO₄, filtered, treated with silica gel and evaporated. The crude product was purified by silica gel chromatography using a MPLC system eluting with a gradient of n-heptane:EtOAc (100:0 to 50:50) to get the title compound as a light yellow solid (105 mg, 13%). MS (ESI): m/z=271.9 [M+H]⁺.

Step 2: tert-Butyl 3-[4-(5-chloro-3-methylsulfonyl-2-pyridyl)phenyl]azetidine-1-carboxylate

A mixture of 2-bromo-5-chloro-3-methylsulfonyl-pyridine (105 mg, 0.31 mmol), tert-butyl 3-[4-(4,4,5,5-tetramethyl-1,3,2-dioxaborolan-2-yl)phenyl]azetidine-1-carboxylate (116.2 mg, 0.31 mmol, CAS RN 1613259-77-0), potassium carbonate (214.6 mg, 1.55 mmol) and tetrakis (triphenylphosphine)palladium (0) (17.9 mg, 0.016 mmol) in THF (1.5 mL) and water (0.15 mL) was vigorously stirred under Ar at 80° C. for 6 h. The reaction mixture was poured on water and EtOAc and the layers were separated. The aqueous layer was extracted twice with EtOAc. The organic layers were washed with water, combined and dried over MgSO₄, filtered, treated with silica gel and evaporated. The crude compound was purified by silica gel chromatography using a MPLC system eluting with a gradient of n-heptane:EtOAc (100:0 to 50:50) to give the desired product as a light brown gum (109 mg, 83%). MS (ESI): m/z=367.1 [M+2H-tBu]⁺.

Step 3:2-[4-(Azetidin-3-yl)phenyl]-5-chloro-3-methylsulfonyl-pyridine; 4-methylbenzenesulfonic Acid

To solution of tert-butyl 3-[4-(5-chloro-3-methylsulfonyl-2-pyridyl)phenyl]azetidine-1-carboxylate (38 mg, 0.090 mmol) in EtOAc (0.350 mL) was added 4-methylbenzenesulfonic acid hydrate (18.8 mg, 0.099 mmol) and the mixture was stirred at reflux for 2 hours. The reaction was allowed to cool down to RT, then evaporated to afford the desired compound as a yellow gum (0.052 g; 99.3%), MS (ESI): m/z=323.1 [M+H]⁺.

### BB66

### 2-(Azetidin-3-yl)-5-(4-chloro-2-fluoro-phenyl)pyridine; 4-methylbenzenesulfonic Acid

Step 1: tert-Butyl 3-[5-(4-chloro-2-fluoro-phenyl)-2-pyridyl]azetidine-1-carboxylate

A mixture of (4-chloro-2-fluorophenyl) boronic acid (55.67 mg, 0.319 mmol, CAS RN 160591-91-3), 3-(5-bromo-2-pyridyl)azetidine-1-carboxylic acid tert-butyl ester (100 mg, 0.319 mmol, CAS RN 1922143-52-9), potassium carbonate (220.64 mg, 1.6 mmol) and tetrakis (triphenylphosphine)palladium (0) (18.45 mg, 0.016 mmol) in THF (1.5 mL) and Water (0.150 mL) was vigorously stirred under argon at 80° C. for 3 hours. Another batch of (4-chloro-2-fluorophenyl) boronic acid (13.92 mg, 0.080 mmol) was added and stirring was continued at 80° C. for another 1.5 hours. The reaction mixture was poured on water and EtOAc and the layers were separated. The aqueous layer was extracted twice with EtOAc. The organic layers were dried over MgSO₄, filtered, treated with silica gel and evaporated. The compound was purified by silica gel chromatography on a 4 g column using an MPLC (ISCO) system eluting with a gradient of n-heptane:EtOAc (100:0 to 50:50) to get the desired product as an off-white solid (0.095 g, 71.3%). MS (ESI): m/z=307.1 [M-C4H8+H]⁺.

Step 2:2-(Azetidin-3-yl)-5-(4-chloro-2-fluoro-phenyl)pyridine; 4-methylbenzenesulfonic Acid

A suspension of tert-butyl 3-[5-(4-chloro-2-fluoro-phenyl)-2-pyridyl]azetidine-1-carboxylate (140 mg, 0.355 mmol) and p-toluenesulfonic acid monohydrate (148.56 mg, 0.781 mmol) in EtOAc (1 mL) was stirred at reflux for 1 hour. The suspension was filtered. The filter cake was washed with EtOAc to get the desired product as a colorless solid (0.216 g, 99.2%). MS (ESI): m/z=263.1 [M+H]⁺.

### BB67

### 2-(Azetidin-3-yl)-5-(2,4-dichlorophenyl)pyridine; 4-methylbenzenesulfonic Acid

Step 1: tert-Butyl 3-[5-(2,4-dichlorophenyl)-2-pyridyl]azetidine-1-carboxylate

The title compound was obtained in analogy to BB65/Step 2 starting from tert-butyl 3-(5-bromo-2-pyridyl)azetidine-1-carboxylate (800 mg, 2.55 mmol, CAS RN 1922143-52-9) and (2,4-dichlorophenyl) boronic acid (633.7 mg, 3.32 mmol, CAS RN 68716-47-2) as a light grey solid (700 mg, 69%). MS (ESI): m/z=379.0 [M+H]⁺.

Step 2:2-(Azetidin-3-yl)-5-(2,4-dichlorophenyl)pyridine; 4-methylbenzenesulfonic Acid

The title compound was obtained in analogy to BB65/Step 3 starting tert-butyl 3-[5-(2,4-dichlorophenyl)-2-pyridyl]azetidine-1-carboxylate (550 mg, 1.45 mmol) as a white solid (870 mg, 93%). MS (ESI): m/z=279.4 [M+H]⁺.

### BB69

### 2-[4-(Azetidin-3-yl)phenyl]-5-(trifluoromethyl)pyrazine; 4-methylbenzenesulfonic Acid

Step 1: tert-Butyl 3-[4-[5-(trifluoromethyl)pyrazin-2-yl]phenyl]azetidine-1-carboxylate

The title compound was obtained in analogy to BB65/Step 2 starting from 2-chloro-5-(trifluoromethyl)pyrazine (60 mg, 0.33 mmol, CAS RN 799557-87-2) and tert-butyl 3-[4-(4,4,5,5-tetramethyl-1,3,2-dioxaborolan-2-yl)phenyl]azetidine-1-carboxylate (124 mg, 0.35 mmol, CAS RN 1613259-77-0) as a colorless solid (101 mg, 78%). MS (ESI): m/z=324.1 [M+2H-tBu]⁺.

Step 2:2-[4-(Azetidin-3-yl)phenyl]-5-(trifluoromethyl)pyrazine; 4-methylbenzenesulfonic Acid

The title compound was obtained in analogy to BB65/Step 3 starting from tert-butyl 3-[4-[5-(trifluoromethyl)pyrazin-2-yl]phenyl]azetidine-1-carboxylate (101 mg, 0.27 mmol) as a colorless solid (107 mg, 86%). MS (ESI): m/z=280.1 [M+H]⁺.

### BB70

### 5-Carbamoyl-4,5-dihydroisoxazole-3-carboxylic Acid

Step 1: Methyl 5-carbamoyl-4,5-dihydroisoxazole-3-carboxylate

To a solution of 2-nitroacetic acid methyl ester (753.88 mg, 6.33 mmol, CAS RN 2483-57-0) and acrylamide (300 mg, 4.22 mmol) in water (13 mL) was added 4.24 M NaOH 4.2 M in water (99.54 μL, 0.422 mmol) and the resulting light yellow solution was stirred in a sealed vial for 2.25 hours at 60° C. The solution was allowed to cool down to RT overnight. As no precipitation occurred, the solution was evaporated. The residue was suspended in EtOAc and filtered to get the desired product as a light yellow solid (0.216 g, 23.8%). MS (ESI): m/z=173.0 [M+H]⁺.

Step 2:5-Carbamoyl-4,5-dihydroisoxazole-3-carboxylic Acid

To a turbid solution of 5-carbamoyl-2-isoxazoline-3-carboxylic acid methyl ester (100 mg, 0.465 mmol) in water (0.700 mL) and 1,4-dioxane (0.300 mL) was added Lithium hydroxide monohydrate (21.45 mg, 0.511 mmol) and the rapidly formed solution was stirred at RT for 2 hours. To the solution was added 2 M HCl 2 M in H₂O (255.61 μL, 0.511 mmol) and the mixture was stirred at RT. Dioxane was evaporated off and eventually also some of the water until a precipitate started to form. The suspension was filtered. The filter cake was washed with a small volume of water to get the desired product as a colorless solid (0.046 g, 62.6%). MS (ESI): m/z=159.0 [M+H]⁺.

### BB71

### 2-[5-(Azetidin-3-yl)-2-pyridyl]-2-azaspiro[3.4]octane; 4-methylbenzenesulfonic Acid

Step 1: tert-Butyl 3-[6-(2-azaspiro[3.4]octan-2-yl)-3-pyridyl]azetidine-1-carboxylate

To a suspension of 3-(6-chloro-3-pyridyl)azetidine-1-carboxylic acid tert-butyl ester (569 mg, 2.12 mmol, CAS RN 870689-19-3) and 2-azaspiro[3.4]octane (235.4 mg, 2.12 mmol, CAS RN 665-41-8) in tert.butanol (12.53 mL) under argon were added x-phos (90.84 mg, 0.191 mmol, CAS RN 564483-18-7), tris(dibenzylideneacetone)dipalladium (0) chloroform adduct (65.75 mg, 0.064 mmol, CAS RN 52522-40-4), and cesium carbonate (2.76 g, 8.47 mmol) and the mixture was heated in a microwave oven at 110° C. for 0.5 hours and 0.5 hours at 100° C. The reaction was heated in the microwave for 1 hour at 90° C. The mixture was filtered, then the filtrate was treated with silica gel and evaporated. The compound was purified by silica gel chromatography on a 40 g column using an MPLC (ISCO) system eluting with a gradient of n-heptane:EtOAc (75:25 to 25:75) to get the desired compound as a yellow solid (0.150 g; 19.7%). MS (ESI): m/z=344.2 [M+H]⁺.

Step 2:2-[5-(Azetidin-3-yl)-2-pyridyl]-2-azaspiro[3.4]octane; 4-methylbenzenesulfonic Acid

The title compound was obtained in analogy to BB65/Step 3 starting from tert-butyl 3-[6-(2-azaspiro[3.4]octan-2-yl)-3-pyridyl]azetidine-1-carboxylate (150 mg, 0.437 mmol) as a light yellow solid (0.262 g; 97.1%), MS (ESI): m/z=244.2 [M+H]⁺.

### BB72

### N-[[2-Fluoro-4-(trifluoromethyl)phenyl]methyl]-N-methyl-azetidin-3-amine; 4-methylbenzenesulfonic Acid

Step 1: tert-Butyl 3-[[2-fluoro-4-(trifluoromethyl)phenyl]methyl-methyl-amino]azetidine-1-carboxylate

A solution of 2-fluoro-4-(trifluoromethyl)benzaldehyde (924.48 mg, 4.81 mmol) and 3-(methylamino)azetidine-1-carboxylic acid tert-butyl ester (0.924 g, 4.96 mmol, CAS RN 454703-20-9) in DCM (10 mL) and acetic acid (595.96 μL, 10.41 mmol) was stirred for 10 minutes at room temperature, then sodium triacetoxy borohydride (1.21 g, 5.73 mmol) was added. The mixture was stirred at RT for 16 hours. The reaction mixture was poured into NaHCO₃ sat. aq. (30 mL) and stirred for 1.5 hours. The mixture was extracted with DCM (2×30 mL), water (30 mL) and NaCl sat.aq. (30 mL). The organic layer was dried over Na2SO4 and concentrated in vacuo to obtain a colorless oil. The crude material was purified by flash chromatography (silica gel, 40 g, 0% to 100% TBME (215 nm) to get the desired compound as a colorless oil (1.07 g; 58.4%). MS (ESI): m/z=363.2 [M+H]⁺.

Step 2: N-[[2-Fluoro-4-(trifluoromethyl)phenyl]methyl]-N-methyl-azetidin-3-amine; 4-methylbenzenesulfonic Acid

The title compound was obtained in analogy to BB65/Step 3 starting from tert-butyl 3-[[2-fluoro-4-(trifluoromethyl)phenyl]methyl-methyl-amino]azetidine-1-carboxylate (1 g, 2.7 mmol) as a colorless solid (1.64 g, 99.6%), MS (ESI): m/z=263.1 [M+H]⁺.

### BB73

### 6-[[6-(Trifluoromethyl)-3-pyridyl]methyl]-2-azaspiro[3.3]heptane; 4-methylbenzenesulfonic Acid

Step 1: tert-Butyl 6-[(4,4,5,5-tetramethyl-1,3,2-dioxaborolan-2-yl)methylene]-2-azaspiro[3.3]heptane-2-carboxylate

A mixture of 2,2,6,6-tetramethylpiperidine (95.9 mL, 568 mmol) in THF (750 mL) was cooled to −30° C. under a N₂ atmosphere. n-BuLi (227 mL, 568 mmol) was added dropwise, and the reaction mixture was stirred at the same temperature for 30 min. Next, the reaction was cooled to −60° C., and a solution of 4,4,5,5-tetramethyl-2-[(4,4,5,5-tetramethyl-1,3,2-dioxaborolan-2-yl)methyl]-1,3,2-dioxaborolane (136 g, 506 mmol) in THF (750 mL) was added dropwise. After stirring for 30 min, a solution of tert-butyl 6-oxo-2-azaspiro[3.3]heptane-2-carboxylate (100 g, 473 mmol) in THF (300 mL) was added in dropwise at −60° C. The reaction mixture was allowed to slowly warm up to 25° C. and stirred at 25° C. for 12 h. The mixture was added H2O (80 mL) slowly and then purified together with an additional batch of equal size by silica gel column (PE/EA=1:0 to 3:1 gradient) to give the title compound as a white solid (220 g, approx 69% yield per batch). 1H NMR (400 MHz, CHLOROFORM-d) δ=5.21-5.16 (m, 1H), 3.99-3.89 (m, 4H), 3.13-2.90 (m, 4H), 1.46-1.41 (m, 9H), 1.26-1.20 ppm (m, 13H).

Step 2: tert-Butyl 6-[[6-(trifluoromethyl)-3-pyridyl]methylene]-2-azaspiro[3.3]heptane-2-carboxylate

To a solution of 5-bromo-2-(trifluoromethyl)pyridine (10.11 g, 44.74 mmol) and tert-butyl 6-[(4,4,5,5-tetramethyl-1,3,2-dioxaborolan-2-yl)methylene]-2-azaspiro[3.3]heptane-2-carboxylate (15.0 g, 44.74 mmol), potassium carbonate (12.37 g, 89.49 mmol) in 1,4-Dioxane (150 mL) and water (30 mL) was added 1,1′-bis(diphenylphosphino) ferrocene-palladium(II) dichloride DCM complex (3.65 g, 4.47 mmol). The mixture was degassed with N₂ for 3 times and stirred at 80° C. under N₂ atmosphere for 12 hours. The residue was purified by flash silica gel chromatography (Eluent of 0~40% EtOAc/Petroleum ether gradient @ 65 mL/min) to give a crude product. The crude product was triturated with petroleum ether at 25° C. for 30 min to give the desired product as a colorless solid (10.0 g, 63.0%). MS (ESI): m/z=355.0 [M+H]⁺.

Step 3: tert-Butyl 6-[[6-(trifluoromethyl)-3-pyridyl]methyl]-2-azaspiro[3.3]heptane-2-carboxylate

To a solution of tert-butyl 6-[[6-(trifluoromethyl)-3-pyridyl]methylene]-2-azaspiro[3.3]heptane-2-carboxylate (26.3 g, 74.22 mmol) in EtOAc (260 mL) was added wet Pd/C (8.7 g, 0.1 eq), and the mixture was stirred at 25° C. under H₂ atmosphere (balloon) for 1.5 h. The mixture was then filtered and the filtrate was concentrated to get the desired product as a colorless solid (26.0 g, 98.3%). ¹H NMR (400 MHz, CHLOROFORM-d) δ=8.50 (s, 1H), 7.60 (d, J=1.4 Hz, 2H), 3.92 (s, 2H), 3.83 (s, 2H), 2.76 (d, J=7.5 Hz, 2H), 2.43 (spt, J=7.8 Hz, 1H), 2.34-2.25 (m, 2H), 1.94-1.85 (m, 2H), 1.47-1.39 (m, 10H).

Step 4:6-[[6-(Trifluoromethyl)-3-pyridyl]methyl]-2-azaspiro[3.3]heptane; 4-methylbenzenesulfonic Acid

To a solution of tert-butyl 6-[[6-(trifluoromethyl)-3-pyridyl]methyl]-2-azaspiro[3.3]heptane-2-carboxylate (2650.0 mg 7.44 mmol), in EtOAc (15 mL) was added p-toluenesulfonic acid (2817.01 mg, 16.36 mmol) at 20° C. The mixture was stirred at 80° C. for 12 h. The reaction was concentrated under reduced pressure to give a residue. To the residue was added deionized water and it was lyophilized to give the desired product as a white solid (2941.0 mg, 65.3%). MS (ESI): m/z=257.2 [M+H]⁺.

### BB74

### 3-[4-[2-Methylsulfonyl-4-(trifluoromethyl)phenyl]phenyl]azetidine; 4-methylbenzenesulfonic Acid

Step 1: tert-Butyl 3-[4-[2-methylsulfonyl-4-(trifluoromethyl)phenyl]phenyl]azetidine-1-carboxylate

A suspension of 3-[4-(4,4,5,5-tetramethyl-1,3,2-dioxaborolan-2-yl)phenyl]azetidine-1-carboxylic acid tert-butyl ester (0.070 g, 0.185 mmol, CAS RN 1613259-77-0), 1-bromo-2-mesyl-4-(trifluoromethyl)benzene (56.1 mg, 0.185 mmol, CAS RN 1215205-98-3) and potassium carbonate (127.91 mg, 0.925 mmol) in THF (0.600 mL) and water (0.060 mL) was purged with argon. To the mixture was added palladiumtetrakis (10.69 mg, 0.009 mmol) and the mixture was stirred at 80° C. in a sealed tube overnight. The mixture was diluted with EtOAc and water and the layers were separated. The aqueous layer was extracted twice with EtOAc. The organic layers were dried over MgSO₄, filtered, treated with silica gel and evaporated. The compound was purified by silica gel chromatography on a 4 g column using an MPLC (ISCO) system eluting with a gradient of n-heptane:EtOAc (100:0 to 50:50) to get the desired product as a colorless foam (0.054 g; 64.0%). MS (ESI): m/z=400.1 [M-C4H8+H]⁺.

Step 2:3-[4-[2-Methylsulfonyl-4-(trifluoromethyl)phenyl]phenyl]azetidine; 4-methylbenzenesulfonic Acid

The title compound was obtained in analogy to BB65/Step 3 starting from tert-Butyl 3-[4-[2-methylsulfonyl-4-(trifluoromethyl)phenyl]phenyl]azetidine-1-carboxylate (0.054 g, 0.11 mmol) as a colorless solid (0.063 g, 86.6%), MS (ESI): m/z=356.1 [M+H]⁺.

### BB75

### 1-[4-(Azetidin-3-yl)phenyl]-3-(difluoromethyl)-5-methyl-pyrazole; 4-methylbenzenesulfonic Acid

Step 1:1-(4-Bromophenyl)-3-(difluoromethyl)-5-methyl-pyrazole

In a dry flask equipped with air flow, copper (II) acetate (406.99 mg, 2.24 mmol) was added to a mixture of 3-(difluoromethyl)-5-methyl-1H-pyrazole (219.3 mg, 1.66 mmol, CAS RN 936033-61-3) and 4-bromophenylboronic acid (500.06 mg, 2.49 mmol) in DCM (16.5 mL), and the reaction was stirred at RT for 2 days. The reaction was filtered and washed with DCM, then the filtrate was isolated and treated with silica gel. The compound was purified by silica gel chromatography on a 120 g column using an MPLC (ISCO) system eluting with DCM to get the desired compound as a colourless oil (0.099 g, 18.7%). MS (ESI): m/z=287.0 [M+H]⁺.

Step 2: tert-Butyl 3-[4-[3-(difluoromethyl)-5-methyl-pyrazol-1-yl]phenyl]azetidine-1-carboxylate

The title compound was obtained in analogy to BB 9/Step 1 starting from 1-(4-bromophenyl)-3-(difluoromethyl)-5-methyl-pyrazole as a colorless solid. MS (ESI): m/z=308.1 [M-C4H8+H]⁺.

### Step 3:1-[4-(Azetidin-3-yl)phenyl]-3-(difluoromethyl)-5-methyl-pyrazole; 4-methylbenzenesulfonic Acid

The title compound was obtained in analogy to BB65/Step 3 starting from tert-Butyl 3-[4-[2-methylsulfonyl-4-(trifluoromethyl)phenyl]phenyl]azetidine-1-carboxylate (0.036 g, 0.099 mmol) as a light brown solid (0.064 g, 88.5%), MS (ESI): m/z=246.2 [M+H]⁺.

### BB76

### 1-[4-(Azetidin-3-yl)phenyl]-3-(trifluoromethyl)-5-methyl-pyrazole; 4-methylbenzenesulfonic Acid

Step 1:1-(4-Bromophenyl)-3-(trifluoromethyl)-5-methyl-pyrazole

The title compound was obtained in analogy to BB 75/Step 1 starting from 5-methyl-3-(trifluoromethyl)-1H-pyrazole (CAS RN 10010-93-2) as a colorless oil. MS (ESI): m/z=305.0 [M+H]⁺.

Step 2: tert-Butyl 3-[4-[3-(trifluoromethyl)-5-methyl-pyrazol-1-yl]phenyl]azetidine-1-carboxylate

The title compound was obtained in analogy to BB 9/Step 1 starting from 1-(4-bromophenyl)-3-(trifluoromethyl)-5-methyl-pyrazole as an orange oil. MS (ESI): m/z=326.2 [M-C4H8+H]⁺.

Step 3:1-[4-(Azetidin-3-yl)phenyl]-3-(trifluoromethyl)-5-methyl-pyrazole; 4-methylbenzenesulfonic Acid

The title compound was obtained in analogy to BB65/Step 3 starting from tert-butyl 3-[4-[3-(trifluoromethyl)-5-methyl-pyrazol-1-yl]phenyl]azetidine-1-carboxylate as a colorless solid, MS (ESI): m/z=282.2 [M+H]⁺.

### BB77

### 1-[4-(Azetidin-3-yl)phenyl]-3,5-diisopropyl-pyrazole; 4-methylbenzenesulfonic Acid

Step 1:1-(4-Bromophenyl)-3,5-diisopropyl-pyrazole

The title compound was obtained in analogy to BB 75/Step 1 starting from 3,5-diisopropyl-1H-pyrazole (CAS RN 17536-00-4) as a colorless oil. MS (ESI): m/z=309.1 [M+H]⁺.

Step 2: tert-Butyl 3-[4-(3,5-diisopropylpyrazol-1-yl)phenyl]azetidine-1-carboxylate

The title compound was obtained in analogy to BB 9/Step 1 starting from 1-(4-bromophenyl)-3,5-diisopropyl-pyrazole as a light yellow oil. MS (ESI): m/z=384.3 [M+H]⁺.

Step 3:1-[4-(Azetidin-3-yl)phenyl]-3,5-diisopropyl-pyrazole; 4-methylbenzenesulfonic Acid

The title compound was obtained in analogy to BB65/Step 3 starting from tert-butyl 3-[4-(3,5-diisopropylpyrazol-1-yl)phenyl]azetidine-1-carboxylate as a light yellow foam. MS (ESI): m/z=284.3 [M+H]⁺.

### BB78

### 3-[3-(4-Chloro-2-methylsulfonyl-phenyl)-1-bicyclo[1.1.1]pentanyl]azetidine; 4-methylbenzenesulfonic Acid

Step 1: tert-Butyl 3-[3-(4-chloro-2-fluoro-phenyl)-1-bicyclo[1.1.1]pentanyl]azetidine-1-carboxylate

The title compound was obtained in analogy to BB 9/Step 1 starting from tert-butyl 3-(3-iodo-1-bicyclo[1.1.1]pentanyl)azetidine-1-carboxylate (CAS RN 2375261-02-0) and 1-bromo-4-chloro-2-fluoro-benzene as a colorless oil. MS (ESI): m/z=296.0 [M-C4H8+H]⁺.

Step 2: tert-Butyl 3-[3-(4-chloro-2-methylsulfonyl-phenyl)-1-bicyclo[1.1.1]pentanyl]azetidine-1-carboxylate

To a solution of tert-butyl 3-[3-(4-chloro-2-fluoro-phenyl)-1-bicyclo[1.1.1]pentanyl]azetidine-1-carboxylate (300 mg, 0.767 mmol) in DMSO (2.5 mL) was added NaSMe (80.68 mg, 1.15 mmol) and the mixture was stirred at 100° C. for 1 hour. The mixture was allowed to cool down to RT. The reaction mixture was poured on water and EtOAc and the layers were separated. The aqueous layer was extracted twice with EtOAc. The organic layers were washed twice with water, dried over MgSO₄, filtered and evaporated. The crude intermediate was dissolved in MeOH (1.8 mL), water (1.8 mL) and oxone (990.7 mg, 1.61 mmol) and NaHCO₃ (154.73 mg, 1.84 mmol) were added. After 2.75 hours another batch of oxone (235.88 mg, 0.384 mmol) and NaHCO3 (32.23 mg, 0.384 mmol) was added. The mixture was stirred overnight. The mixture was diluted with saturated aqueous NaHCO₃ solution and EtOAc and the layers were separated. The aqueous layer was extracted twice with EtOAc. The organic layers were washed once with water, dried over MgSO₄, filtered and evaporated to get the desired crude product. The crude product was purified by silica gel chromatography on a 12 g column using an MPLC (ISCO) system eluting with a gradient of n-heptane:EtOAc (100:0 to 0:100) to get the desired product P2 as a colorless gum (0.135 g, 42.7%). MS (ESI): m/z=356.0 [M-C4H8+H]⁺.

Step 3:3-[3-(4-Chloro-2-methylsulfonyl-phenyl)-1-bicyclo[1.1.1]pentanyl]azetidine; 4-methylbenzenesulfonic Acid

The title compound was obtained in analogy to BB65/Step 3 starting from tert-butyl 3-[3-(4-chloro-2-methylsulfonyl-phenyl)-1-bicyclo[1.1.1]pentanyl]azetidine-1-carboxylate as a colorless foam. MS (ESI): m/z=312.0 [M+H]⁺.

### BB79

### 3-[3-[2-Methylsulfonyl-4-(trifluoromethyl)phenyl]-1-bicyclo[1.1.1]pentanyl]azetidine; 4-methylbenzenesulfonic Acid

The title compound was obtained in analogy to BB 78/Step 1-3 starting from 1-bromo-2-fluoro-4-(trifluoromethyl)benzene (CAS RN 40161-54-4) as a colorless solid. MS (ESI): m/z=346.1 [M+H]⁺.

### BB80

### 4-(Azetidin-3-yl)-N-[[1-(trifluoromethyl)cyclopropyl]methyl]aniline; 4-methylbenzenesulfonic Acid

Step 1: tert-Butyl 3-[4-[[1-(trifluoromethyl)cyclopropyl]methylamino]phenyl]azetidine-1-carboxylate

To a solution of [1-(trifluoromethyl)cyclopropyl]methanamine; hydrochloride (365.56 mg, 2.08 mmol, CAS RN1783418-59-6), tert-butyl 3-(4-bromophenyl)azetidine-1-carboxylate (500.0 mg, 1.6 mmol, CAS RN 1203681-52-0) and sodium tert-butoxide (615.65 mg, 6.41 mmol) in t-Amyl-OH (2.0 mL) was added tBuXPhos-Pd-G3 (120.01 mg, 0.16 mmolCAS RN 1447963-75-8), the mixture was then stirred under N₂ atmosphere at 110° C. for 12 h. Water (50 mL) was added in the reaction mixture and the aqueous phase was extracted with EtOAc (40 mL) twice. The organic layers were combined and washed with brine and dried over Na2SO4. The organic layer was filtered and concentrated under reduced pressure evaporated to dryness. The mixture was purified by Prep-HPLC (Phenomenex luna C18 150*40 mm*15 μm, water (0.225% FA)-ACN) and lyophilized to get the desired product as a light yellow oil. (300.0 mg, 0.81 mmol, 50.57% yield). MS (ESI): m/z=315.3 [M-C4H8+H]⁺.

Step 1:4-(Azetidin-3-yl)-N-[[1-(trifluoromethyl)cyclopropyl]methyl]aniline; 4-methylbenzenesulfonic Acid

The title compound was obtained in analogy to BB65/Step 3 starting from tert-butyl 3-[4-[[1-(trifluoromethyl)cyclopropyl]methylamino]phenyl]azetidine-1-carboxylate as a light grey solid. MS (ESI): m/z=271.4 [M+H]⁺.

### BB81

### 7-[2-Fluoro-4-(trifluoromethyl) phenoxy]-2-azaspiro[3.5]nonane; 4-methylbenzenesulfonic Acid

Step 1: tert-Butyl 7-[2-fluoro-4-(trifluoromethyl) phenoxy]-2-azaspiro[3.5]nonane-2-carboxylate

To a solution of 7-hydroxy-2-azaspiro[3.5]nonane-2-carboxylic acid tert-butyl ester (670 mg, 2.78 mmol, CAS RN 1363383-18-9) and potassium t-butoxide (373.84 mg, 3.33 mmol) in DMF (5 mL) was added 1,2-difluoro-4-(trifluoromethyl)benzene (530.81 mg, 2.92 mmol, CAS RN 32137-19-2). The mixture was stirred at RT for 2 h. The reaction mixture was poured into AcOET and washed with water and brine. The organic layer was dried over Na2SO4 and concentrated in vacuo. The crude material was purified by flash chromatography (silica gel, 40 g, 0% to 80% AcOEt in heptane) to get the desired product as a colorless solid (743 mg, 63.0%) as white solid MS: 348.3 [M-C4H8+H]⁺.

Step 1:7-[2-Fluoro-4-(trifluoromethyl) phenoxy]-2-azaspiro[3.5]nonane; 4-methylbenzenesulfonic Acid

The title compound was obtained in analogy to BB65/Step 3 starting from tert-butyl 7-[2-fluoro-4-(trifluoromethyl) phenoxy]-2-azaspiro[3.5]nonane-2-carboxylate as a colorless solid. MS (ESI): m/z=304.1 [M+H]⁺.

### BB82

### 5-[4-(Azetidin-3-yl)phenyl]-2-chloro-4-methylsulfonyl-pyridine; 4-methylbenzenesulfonic Acid

Step 1:2-Chloro-5-iodo-4-methylsulfonyl-pyridine

To a suspension of 2-chloro-4-fluoro-5-iodo-pyridine (100 mg, 0.388 mmol, CAS RN 1370534-60-3) in DMSO (0.500 mL) under argon was added sodium; methanesulfinate (39.66 mg, 0.388 mmol) and the suspension was stirred at 75° C. overnight. Further sodium; methanesulfinate (19.83 mg, 0.194 mmol) was added and the reaction left to stir for 1 hour. The reaction was poured on water (10 mL) and extracted using EtOAc (10 mL). The aqueous was extracted a further 2 times with EtOAc (2×10 mL) and the combined organic layers were dried over Na2SO4 then filtered and evaporated to give the desired product as a waxy orange solid (0.109 g, 83.9%). MS (ESI): m/z=317.9 [M+H]⁺.

Step 2: tert-Butyl 3-[4-(6-chloro-4-methylsulfonyl-3-pyridyl)phenyl]azetidine-1-carboxylate

A mixture of 2-chloro-5-iodo-4-methylsulfonyl-pyridine (107.17 mg, 0.321 mmol), 3-[4-(4,4,5,5-tetramethyl-1,3,2-dioxaborolan-2-yl)phenyl]azetidine-1-carboxylic acid tert-butyl ester (120 mg, 0.321 mmol, CAS RN 1613259-77-0), potassium carbonate (221.58 mg, 1.6 mmol) and tetrakis(triphenylphosphine)palladium (0) (18.53 mg, 0.016 mmol) in THF (1.51 mL) and Water (0.151 mL) was vigorously stirred under argon at 80° C. for 16 hours. The reaction mixture was poured on water and EtOAc and the layers were separated. The aqueous layer was extracted twice with EtOAc. The organic layers were combined and dried over MgSO₄, filtered, treated with silica gel and evaporated. The compound was purified by silica gel chromatography on a 4 g column using an MPLC (ISCO) system eluting with a gradient of n-heptane:EtOAc (100:0 to 50:50) to give the desired product as a yellow gum (0.038 g, 27.2%). MS (ESI): m/z=367.1 [M-C4H8+H]⁺.

Step 3:5-[4-(Azetidin-3-yl)phenyl]-2-chloro-4-methylsulfonyl-pyridine; 4-methylbenzenesulfonic Acid

The title compound was obtained in analogy to BB65/Step 3 starting from tert-Butyl 3-[4-(6-chloro-4-methylsulfonyl-3-pyridyl)phenyl]azetidine-1-carboxylate as a yellow gum. MS (ESI): m/z=323.1 [M+H]⁺.

### BB83

### 5-(Azetidin-3-yl)-N-[[1-(trifluoromethyl)cyclopropyl]methyl]pyridin-2-amine; 4-methylbenzenesulfonic Acid

Step 1: tert-butyl 3-(6-Bromo-3-pyridyl)azetidine-1-carboxylate

The stirred mixture of tert-butyl 3-(p-tolylsulfonylhydrazone)azetidine-1-carboxylate (68.0 g, 200 mmol, CAS RN 1510865-66-3), 2-bromopyridine-5-boronic acid (53.8 g, 266 mmol) and potassium carbonate (41.5 g, 301 mmol) in dry 1,4-Dioxane (2800 mL) were refluxed for 24 h (Argon). Then obtained precipitate was filtered off and filtrate was evaporated to dryness. The obtained residue was partioned between TBME (2000 mL) and water (500 mL). The organic layer was washed with brine (100 mL), dried over Na2SO4 and evaporated in vacuum. The crude product was purified by flash chromatography to obtain the title compound (13.8 g, 21% yield) as a light yellow oil. MS (ESI): m/z=313.0 [M+H]⁺.

Step 2: tert-Butyl 3-[6-[[1-(trifluoromethyl)cyclopropyl]methylamino]-3-pyridyl]azetidine-1-carboxylate

The mixture of tert-butyl 3-(6-bromo-3-pyridyl)azetidine-1-carboxylate (7.0 g, 22.4 mmol), [1-(trifluoromethyl)cyclopropyl]methanamine; hydrochloride (5.89 g, 33.5 mmol), tris (dibenzylideneacetone)dipalladium (1.02 g, 1.12 mmol), XantPhos (1.03 g, 1.79 mmol) and sodium tert-butoxide (6.44 g, 67.1 mmol) was sealed and stirred in degassed Toluene (100 mL) at 100° C. for 24 h (Argon atmosphere). Then RM was cooled to RT and filtered through a pad of SiO2, washed with toluene (300 mL) and concentrated in vacuo. Purification by FC (SiO2; hexane/MTBE) gave the title compound (5.5 g, 63% yield) as orange crystals. MS (ESI): m/z=372.2 [M+H]⁺.

Step 3:5-(Azetidin-3-yl)-N-[[1-(trifluoromethyl)cyclopropyl]methyl]pyridin-2-amine; 4-methylbenzenesulfonic Acid

The title compound was obtained in analogy to BB65/Step 3 starting from tert-butyl 3-[6-[[1-(trifluoromethyl)cyclopropyl]methylamino]-3-pyridyl]azetidine-1-carboxylate as a light yellow solid. MS (ESI): m/z=272.2 [M+H]⁺.

### BB84

### 4-[[5-(Trifluoromethyl)pyrazin-2-yl]amino]cubane-1-carboxylic Acid

Step 1: Methyl 4-[[5-(trifluoromethyl)pyrazin-2-yl]amino]cubane-1-carboxylate

To a solution of 4-(tert-butoxycarbonylamino) cubane-1-carboxylic acid methyl ester (42.3 mg, 0.153 mmol, CAS RN 883554-71-0) in DCM (0.423 mL) was added TFA (423 μL, 5.49 mmol) and the reaction was allowed to stir for 1 h. Then, TFA and DCM were evaporated under reduced pressure (TFA azeotropically removed with 3×1 mL DCM). The residue was taken up in DMF, extra dry (1.53 mL). Then, DIEA (59.14 mg, 79.92 μL, 0.458 mmol, 3.000 eq) was added as well as 2-fluoro-5-(trifluoromethyl)pyrazine (27.78 μL, 0.229 mmol). The reaction vessel was flushed with nitrogen before being sealed and allowed to stir over night. The reaction mixture was diluted with DCM and water. The phases were separated and the aqueous layer was extracted with DCM. The combined organic layers were washed with brine, dried over Na2SO4, filtered and evaporated under reduced pressure to get the desire product as a yellow gum (52 mg, 100%). MS (ESI): m/z=324.1 [M+H]⁺.

Step 2:4-[[5-(Trifluoromethyl)pyrazin-2-yl]amino]cubane-1-carboxylic Acid

To a solution of 4-[[5-(trifluoromethyl)pyrazin-2-yl]amino]cubane-1-carboxylic acid methyl ester (52 mg, 0.153 mmol) in THF (1.31 mL) was added water (0.218 mL) and LiOH monohydrate (19.24 mg, 0.458 mmol). The mixture was sonicated and allowed to stir for 1 h. EtOAc was added and 2 M HCl. The phases were separated and the organic layer was washed with brine, dried over Na2So4, filtered and evaporated under reduced pressure to get the desired product as ay yellow solid (36.5 mg, 73.3%). MS (ESI): m/z=308.1 [M−H]⁻.

### BB85

### 4-[[2-Fluoro-4-(trifluoromethoxy)phenyl]methoxy]piperidine

Step 1: tert-Butyl 4-[[2-fluoro-4-(trifluoromethoxy)phenyl]methoxy]piperidine-1-carboxylate

To a solution of 4-hydroxypiperidine-1-carboxylic acid tert-butyl ester (1.5 g, 7.45 mmol, CAS RN 109384-19-2) in dry THF (3 mL) was added potassium tert-butoxide 1 M solution in THF (1.04 ml, 1.04 mmol) and the turbid reaction mixture was stirred at RT for 15 min followed by addition of 1-(bromomethyl)-2-chloro-4-fluorobenzene (222 mg, 994 μmol, CAS RN 1240257-47-9). The reaction mixture was then stirred at RT for 3 hours. The reaction mixture was poured on water and EtOAc and the layers were separated. The organic layer was washed twice with water and dried over MgSO₄, filtered and evaporated to give the desired product as a colorless oil (2.7 g, 73.6%). The product was used as is for next step.

### Step 2:4-[[2-Fluoro-4-(trifluoromethoxy)phenyl]methoxy]Piperidine

To a solution of tert-butyl 4-[[2-fluoro-4-(trifluoromethoxy)phenyl]methoxy]piperidine-1-carboxylate (750 mg, 1.91 mmol) in DCM (11.76 mL) and MeOH (2.35 mL), 4 M HCl 4 M in Dioxan (752.94 μL, 3.01 mmol) was added and the mixture was stirred overnight at RT. The mixture was evaporated to give the desired product as a colorless solid (0.5 g, 80.4%). The product was used as is for next step.

### BB86

### 1-[(3S)-Pyrrolidin-3-yl]tetrazole

### Step 1:1-[(3S)-1-Benzylpyrrolidin-3-yl]tetrazole

To a solution of (3S)-1-benzylpyrrolidin-3-yl amine (6.0 g, 34.0 mmol, CAS RN 224715-38-7) and triethoxymethane (33.9 mL, 204.24 mmol) in acetic acid (250 mL) was added sodium azide (13.3 g, 204.24 mmol, CAS RN 26628-22-8). The resulting light brown mixture was heated up to 100° C. and stirred for 4 h. It was cooled to RT. The volatiles were removed in vacuo until a volume of approx. 100 mL. The residue was transferred to an Erlenmeyer-flask. Under stirring ethylacetate (250 ml) and then 300 ml of NaHCO₃ (sat. aq.) were added. The mixture was transferred to a separatory funnel and the mixture was extracted with EtOAc (total: 3*250 mL), sodium bicarbonate (sat. aq. 2*200 ml) and brine (1*200 ml). The combined organics were dried over sodium sulfate, filtered, and concentrate to dryness under vacuum. The crude was purified by flash chromatography to obtain the title compound (4.34 g, 55.1% yield) as a light yellow oil. MS GC (EI+): m/z=229.1 [M+]⁺.

### Step 2:1-[(3S)-Pyrrolidin-3-yl]tetrazole

Under Argon, to a solution of 1-[(3S)-1-benzylpyrrolidin-3-yl]tetrazole (4.34 g, 18.7 mmol) in MeOH (100 mL) was added Pd/C 10% (1200 mg, 1.128 mmol) at RT. The mixture was evacuated and backfilled with hydrogen. This was made three times. The suspension was stirred under an hydrogen balloon atmosphere for 20 h. The mixture was degassed with argon and filtered. The filter cake was washed with MeOH (3*15 ml) and the solution was concentrated to afford the title compound (2.45 g, 93.8%) as colorless oil MS (ESI): m/z=140.1 [M+H]⁺.

### BB87

### 1-[(3R)-Pyrrolidin-3-yl]tetrazole

### Step 1:1-[(3R)-1-Benzylpyrrolidin-3-yl]tetrazole

The title compound was obtained in analogy to BB86 step 1, starting from (3R)-1-benzylpyrrolidin-3-yl amine (6.0 g, 34.04 mmol, CAS 114715-39-8) to afford the title compound (3810 mg, 48.33%) as orange oil MS (ESI): m/z=230.2 [M+H]⁺.

### Step 2:1-[(RS)-Pyrrolidin-3-yl]tetrazole

The title compound was obtained in analogy to BB86 step 2, starting from 1-[(3R)-1-benzylpyrrolidin-3-yl]tetrazole (3.63 g, 15.83 mmol) to afford the title compound (2.0 g, 86.2%) as a light yellow oil MS (ESI): m/z=140.1 [M+H]⁺.

### BB88

### 2-(Azetidin-3-yl)-5-(2,4-dichlorophenoxy)pyrazine; 4-methylbenzenesulfonic Acid

Step 1:2-Bromo-5-(2,4-dichlorophenoxy)pyrazine

To a solution of 2,4-dichlorophenol (652 mg, 4 mmol, CAS 120-83-2) in DMF (15 mL) were added 2,5-dibromopyrazine (970.55 mg, 4.08 mmol, CAS 23229-26-7) and K₂CO₃ (1.11 g, 8 mmol, CAS584-08-7) at RT. The yellow mixture was stirred at 110° C. for 1.5 h. The mixture was allowed to cool down to RT and filtered. The filtrate solution was dried at the high vacuum. The crude was purified by flash chromatography with heptane and EtOAc, to obtain the title compound (1220 mg, 95.3%) as white solid MS (ESI): m/z=319.0 [M+H]⁺.

Step 2:3-[5-(2,4-Dichlorophenoxy)pyrazin-2-yl]azetidine-1-carboxylic Acid Tert-Butyl Ester

The product was obtained in analogy to BB9, step 1 starting from 2-bromo-5-(2,4-dichlorophenoxy)pyrazine (800 mg, 2.5 mmol) and tert-butyl 3-bromoazetidine-1-carboxylate (885.49 mg, 3.75 mmol) to get the title compound (450 mg, 37.24%) as an off-white solid. MS (ESI): m/z=340.1 [M-C4H8+H]⁺.

Step 3:2-(Azetidin-3-yl)-5-(2,4-dichlorophenoxy)pyrazine; 4-methylbenzenesulfonic Acid

The product was obtained in analogy to BB24, step 5 starting from: 3-[5-(2,4-dichlorophenoxy)pyrazin-2-yl]azetidine-1-carboxylic acid tert-butyl ester to get the title compound as a light brown solid. MS (ESI): m/z=296.1 [M+H]⁺.

### BB89

### 6-[[5-(Trifluoromethyl)pyrazin-2-yl]methyl]-2-azaspiro[3.3]heptane; 4-methylbenzenesulfonic Acid

Step 1:6-[[5-(Trifluoromethyl)pyrazin-2-yl]methylene]-2-azaspiro[3.3]heptane-2-carboxylic Acid Tert-Butyl Ester

A solution of 6-formyl-2-azaspiro[3.3]heptane-2-carboxylic acid tert-butyl ester (300 mg, 1.33 mmol; CAS RN 1440960-67-7) and p-toluenesulfonhydrazide (280 mg, 1.5 mmol; CAS RN 1576-35-8) in 1,4-dioxane (8.88 mL) was stirred at 80° C. under Argon atmosphere for 2 h. Then the reaction mixture was allowed to cool down to RT. To the mixture were added 2-bromo-5-(trifluoromethyl)pyrazine (362.72 mg, 1.6 mmol; CAS 1196152-38-1), lithium tert-butoxide (373.1 mg, 4.66 mmol; CAS 1196152-38-1) and bis(triphenylphosphine)palladium (ii) chloride (93.47 mg, 0.133 mmol; CAS 13965-03-2). The mixture was stirred at 100° C. under Argon overnight. The cold reaction mixture was diluted with EtOAc, washed with water and brine. The organic phase was collected, dried over Na2SO4 and concentrated. The crude was purified by flash chromatography with heptane and EtOAc, to obtain the title compound (79.5 mg, 15.6%) as yellow solid MS (ESI): m/z=300.1 [M-C4H8+H]⁺.

Step 2:6-[[5-(Trifluoromethyl)pyrazin-2-yl]methyl]-2-azaspiro[3.3]heptane-2-carboxylic Acid Tert-Butyl Ester

To a solution of 6-[[5-(trifluoromethyl)pyrazin-2-yl]methylene]-2-azaspiro[3.3]heptane-2-carboxylic acid tert-butyl ester (55 mg, 0.155 mmol) in EtOAc (5 mL) was added palladium Pd/C 10% (16.47 mg, 0.016 mmol) and the mixture was stirred at RT under H₂ overnight. After rechange from hydrogen to argon the Pd/C 10% was removed by filtration, washed with MeOH and the solution was concentrated to afford the title compound (46 mg, 83.7%) as yellow oil MS (ESI): m/z=302.1 [M-C4H8+H]⁺.

Step 3:6-[[5-(Trifluoromethyl)pyrazin-2-yl]methyl]-2-azaspiro[3.3]heptane; 4-methylbenzenesulfonic Acid

The product was obtained in analogy to BB24, step 5, starting from 6-[[5-(trifluoromethyl)pyrazin-2-yl]methyl]-2-azaspiro[3.3]heptane-2-carboxylic acid tert-butyl ester to get the title compound as a light yellow waxy solid. MS (ESI): m/z=258.1 [M+H]⁺.

### BB90

### 2-[4-(Azetidin-3-yl) phenoxy]-5-(trifluoromethyl)pyrazine; 4-methylbenzenesulfonic Acid

Step 1: tert-Butyl 3-(4-hydroxyphenyl)azetidine-1-carboxylate

The title compound was obtained in analogy to BB 9/Step 1 starting from tert-butyl 3-bromoazetidine-1-carboxylate (CAS RN: 1064194-10-0) and 4-bromophenol (CAS RN: 106-41-2 as a light yellow solid. MS (ESI): m/z=194.0 [M-C4H8+H]⁺.

Step 2: tert-Butyl 3-[4-[5-(trifluoromethyl)pyrazin-2-yl]oxyphenyl]azetidine-1-carboxylate

To a solution of tert-butyl 3-(4-hydroxyphenyl)azetidine-1-carboxylate (450 mg, 1.8 mmol) and potassium carbonate (748.4 mg, 5.41 mmol) in DMSO (6.75 mL) was added 2-bromo-5-(trifluoromethyl)pyrazine (450.66 mg, 1.99 mmol). The mixture was heated to 100° C. and stirred for one hour at this temperature, before being cooled down and diluted with EtOAc. The organic layer was washed with water and brine, dried over MgSO4, filtered, and evaporated. Purification by flash chromatography (SiO₂; heptane/MTBE) gave the title compound (691 mg, 94.9% yield) as a light yellow solid. MS (ESI): m/z=340.1 [M-C4H8+H]⁺.

Step 3:2-[4-(Azetidin-3-yl) phenoxy]-5-(trifluoromethyl)pyrazine, 4-methylbenzenesulfonic Acid

The product was obtained in analogy to BB24, step 5, starting from tert-butyl 3-[4-[5-(trifluoromethyl)pyrazin-2-yl]oxyphenyl]azetidine-1-carboxylate to get the title compound as a white solid. MS (ESI): m/z=296.1 [M+H]⁺.

### BB91

### 2-[4-(Azetidin-3-yl) phenoxy]-4-cyclopropyl-pyrimidine; 4-methylbenzenesulfonic Acid

Step 1: tert-Butyl 3-[4-(4-cyclopropylpyrimidin-2-yl)oxyphenyl]azetidine-1-carboxylate

To a solution of tert-butyl 3-(4-hydroxyphenyl)azetidine-1-carboxylate (700.0 mg, 2.81 mmol) (BB90 Step 1) and Cs₂CO₃ (1829.5 mg, 5.62 mmol) in DMF (15 mL), 2-chloro-4-cyclopropyl-pyrimidine (520.89 mg, 3.37 mmol, CAS RN 954237-31-1) was added and stirred at 50° C. for 12 h. The reaction mixture was poured into water (50 mL) and extracted with EtOAc (50 mL three times), the combined organic phase was washed with brine, dried over Na2SO4, and concentrated, the residue was purified by silica gel column (PE:EtOAc=20:1~10:1) to get the title compound (840.0 mg, 81.42% yield) as a off-white solid. MS (ESI): m/z=312.4 [M-C4H8+H]⁺.

Step 2:2-[4-(Azetidin-3-yl) phenoxy]-4-cyclopropyl-pyrimidine; 4-methylbenzenesulfonic Acid

The product was obtained in analogy to BB24, step 5, starting from tert-butyl 3-[4-(4-cyclopropylpyrimidin-2-yl)oxyphenyl]azetidine-1-carboxylate to get the title compound as a off-white solid. MS (ESI): m/z=268.4 [M+H]⁺.

### BB92

### 5-(2-Azaspiro[3.3]heptan-6-ylmethyl)-2-(trifluoromethoxy)benzoic acid methyl ester; 4-methylbenzenesulfonic Acid

Step 1:6-[3-Carbomethoxy-4-(trifluoromethoxy)benzylidene]-2-azaspiro[3.3]heptane-2-carboxylic Acid Tert-Butyl Ester

In a 25 mL flask were dissolved 5-bromo-2-(trifluoromethoxy)benzoic acid methyl ester (55.9 mg, 0.187 mmol, CAS RN 773874-13-8) and 6-[(4,4,5,5-tetramethyl-1,3,2-dioxaborolan-2-yl)methylene]-2-azaspiro[3.3]heptane-2-carboxylic acid tert-butyl ester, (57 mg, 0.170 mmol, BB73 step 1) in a degassed solution of 1,4-dioxane (8.5 mL) and water (0.85 mL). The reaction mixture was degassed 5 min again before addition of 1,1′-bis(di-tert-butylphosphino) ferrocene-palladium dichloride (5.54 mg, 0.009 mmol, CAS RN 95408-45-0) followed by tripotassium phosphate (72.2 mg, 0.34 mmol, CAS RN 7778-53-2). The reaction mixture was stirred at RT for 3 hours. The cold reaction mixture was poured into EtOAc, washed with water. The aqueous layer was extracted back twice. Combined organic layers was washed with brine, dried over Na2SO₄, filtered and concentrated under reduced pressure. The resulting crude was then purified by flash chromatography (Silica gel, 4 g) using a gradient Hept/EtOAc to afford the title compound as a light brown oil. MS (ESI): m/z=372.1 [M-C4H8+H]⁺.

Step 2:6-[3-Carbomethoxy-4-(trifluoromethoxy)benzyl]-2-azaspiro[3.3]heptane-2-carboxylic Acid Tert-Butyl Ester

In a three necked flask was dissolved 6-[3-carbomethoxy-4-(trifluoromethoxy)benzylidene]-2-azaspiro[3.3]heptane-2-carboxylic acid tert-butyl ester (42 mg, 0.098 mmol) in MeOH (3.3 mL) and THF (1.6 mL). The reaction mixture was degassed with Argon during 10 min. Then, platinum (IV) oxide (4.46 mg, 0.02 mmol) was added to the reaction mixture. The argon balloon was removed and replaced by hydrogen. A hydrogen flow was used during 5 min and then the stirring reaction was kept under hydrogen atmosphere for one hour. The reaction mixture was filtrated, solvent removed under reduced pressure to afford the desired compound as a colorless oil. MS (ESI): m/z=374.1 [M-C4H8+H]⁺.

Step 3:5-(2-Azaspiro[3.3]heptan-6-ylmethyl)-2-(trifluoromethoxy)benzoic acid methyl ester; 4-methylbenzenesulfonic Acid

The product was obtained in analogy to BB24, step 5, starting from 6-[3-carbomethoxy-4-(trifluoromethoxy)benzyl]-2-azaspiro[3.3]heptane-2-carboxylic acid tert-butyl ester to get the title compound as a off-white solid. MS (ESI): m/z=330.1 [M+H]⁺.

### B93

### 7-[[2-Fluoro-4-(trifluoromethyl)phenyl]methyl]-2,7-diazaspiro[3.5]nonane; 4-methylbenzenesulfonic Acid

Step 1: tert-Butyl 7-[[2-fluoro-4-(trifluoromethyl)phenyl]methyl]-2,7-diazaspiro[3.5]nonane-2-carboxylate

To a solution of 2-fluoro-4-(trifluoromethyl)benzaldehyde (1 g, 5.21 mmol, CAS RN 89763-93-9) and 2,7-diazaspiro[3.5]nonane-2-carboxylic acid tert-butyl ester (1.18 g, 5.21 mmol, CAS RN 236406-55-6) in DCM (10 mL) was added sodium triacetoxy borohydride (1.21 g, 5.73 mmol) and acetic acid (595.96 μL, 10.41 mmol). The mixture was stirred at RT for 4 h. The reaction mixture was poured into AcOET/THF 2:1 and aqueous saturated NaHCO₃ solution and the layers were separated. The organic layer was washed with water and brine. The organic layer was dried over Na2SO4 and concentrated in vacuo. The crude material was purified by flash chromatography (silica gel, 40 g, 0% to 30% AcOEt in heptane) to afford the desired product (1.23 g, 55.78%) as white solid MS: 403.4 [M+H]⁺,

Step 2:7-[[2-Fluoro-4-(trifluoromethyl)phenyl]methyl]-2,7-diazaspiro[3.5]nonane; 4-methylbenzenesulfonic Acid

The product was obtained in analogy to BB24, step 5, starting from tert-butyl 7-[[2-fluoro-4-(trifluoromethyl)phenyl]methyl]-2,7-diazaspiro[3.5]nonane-2-carboxylate to get the title compound as a colorless solid. MS (ESI): m/z=303.1 [M+H]⁺.

### BB94

### 6-[[2-Fluoro-4-(trifluoromethyl)phenyl]methyl]-2-azaspiro[3.3]heptane; 4-methylbenzenesulfonic Acid

Step 1: tert-Butyl 6-[[2-fluoro-4-(trifluoromethyl)phenyl]methylene]-2-azaspiro[3.3]heptane-2-carboxylate

A mixture of tert-butyl 6-[(4,4,5,5-tetramethyl-1,3,2-dioxaborolan-2-yl)methylene]-2-azaspiro[3.3]heptane-2-carboxylate (1.0 g, 2.98 mmol, CAS RN 2763647-64-7), 4-bromo-3-fluorobenzotrifluoride (0.87 g, 3.58 mmol), 1,1′-bis(diphenylphosphino) ferrocene-palladium(II) dichloride DCM complex (0.49 g, 0.6 mmol) and potassium carbonate (0.82 g, 5.97 mmol) in 1,4-Dioxane (15 mL) and water (3 mL) was heated to 120° C. under argon for 16 hours. The reaction mixture was concentrated under reduced pressure. The residue was partitioned between EtOAc and water. The organic layer was washed with brine. The extract was dried over sodium sulfate, filtered through a thin layer of silica gel and evaporated to get the desired product as a dark brown oil (1.4 g, 88.4% yield). The product was used as is for next step.

Step 2: tert-Butyl 6-[[2-fluoro-4-(trifluoromethyl)phenyl]methyl]-2-azaspiro[3.3]heptane-2-carboxylate

A mixture of tert-butyl 6-[[2-fluoro-4-(trifluoromethyl)phenyl]methylene]-2-azaspiro[3.3]heptane-2-carboxylate (2.2 g, 5.92 mmol) and platinum (0.22 g, 0.11 mmol) in EtOAc (40 mL) was stirred in an autoclave for 24 h under 40 bar of hydrogen. The reaction mixture was filtered through a thin layer of silica hel, and concentrated to get the desired product as a light yellow solid (2.05 g, 5.49 mmol, 88.04% yield). Used as is for next step.

Step 3:6-[[2-Fluoro-4-(trifluoromethyl)phenyl]methyl]-2-azaspiro[3.3]heptane; 4-methylbenzenesulfonic Acid

The product was obtained in analogy to BB24, step 5, starting from tert-butyl 6-[[2-fluoro-4-(trifluoromethyl)phenyl]methyl]-2-azaspiro[3.3]heptane-2-carboxylate to get the title compound as a colorless solid. MS (ESI): m/z=274.0 [M+H]⁺.

### BB95

### N-[6-(Trifluoromethyl)-3-pyridyl]-2-azaspiro[3.5]nonan-7-amine; 4-methylbenzenesulfonic Acid

Step 1: tert-Butyl 7-[[5-(trifluoromethyl)-2-pyridyl]amino]-2-azaspiro[3.5]nonane-2-carboxylate

To a solution of 7-amino-2-azaspiro[3.5]nonane-2-carboxylic acid tert-butyl ester (0.560 g, 2.33 mmol, CAS RN 1408075-19-3) and DIEA (813.87 μL, 4.66 mmol) in DMSO (3 mL) was added 2-chloro-5-(trifluoromethyl)pyridine (444.14 mg, 2.45 mmol, CAS RN 52334-81-3). The mixture was stirred at 100° C. for 15 h. The reaction mixture was poured into AcOET and washed with water and brine. The organic layer was dried over Na2SO4 and concentrated in vacuo. The crude material was purified by flash chromatography (silica gel, 40 g, 0% to 30% AcOEt in heptane) to afford (331 mg, 35.02%) as white solid MS: 386.3 [M+H]⁺.

Step 2: N-[6-(Trifluoromethyl)-3-pyridyl]-2-azaspiro[3.5]nonan-7-amine; 4-methylbenzenesulfonic Acid

The product was obtained in analogy to BB24, step 5, starting from tert-butyl 7-[[5-(trifluoromethyl)-2-pyridyl]amino]-2-azaspiro[3.5]nonane-2-carboxylate to get the title compound as a colorless solid. MS (ESI): m/z=286.1 [M+H]⁺.

### BB96

### 3-[[2-Fluoro-4-(trifluoromethylsulfonyl)phenyl]methoxy]azetidine; 4-methylbenzenesulfonic Acid

Step 1: tert-Butyl 3-[(2-fluoro-4-iodo-phenyl)methoxy]azetidine-1-carboxylate

To a solution of tert-butyl 3-hydroxyazetidine-1-carboxylate (2.92 g, 16.83 mmol, CAS RN 141699-55-0) in THF (50 mL) were added potassium tert-butoxide (3.78 g, 33.66 mmol) and 1-(bromomethyl)-2-fluoro-4-iodo-benzene (5.3 g, 16.83 mmol CAS RN: 85510-81-2), at 25° C. under Ar. The mixture was stirred for 12 h at 30° C., before being evaporated. Purification by FC (SiO2; PE/EtOAc) and RP-HPLC gave the title compound (3.0 g, 44% yield) as a colorless oil. 1H NMR (400 MHz, CDCl3) δ=7.51 (dd, J=1.4, 8.1 Hz, 1H), 7.43 (dd, J=1.6, 9.1 Hz, 1H), 7.14 (t, J=7.8 Hz, 1H), 4.46 (s, 2H), 4.32 (tdd, J=2.0, 4.3, 6.4 Hz, 1H), 4.11-4.05 (m, 2H), 3.89-3.83 (m, 2H), 1.44 ppm (s, 9H).

Step 2: tert-Butyl 3-[[2-fluoro-4-(trifluoromethylsulfanyl)phenyl]methoxy]azetidine-1-carboxylate

To a 40 mL vial equipped with a magnetic stir bar were added trifluoromethylsulfanylsilver (769.63 mg, 3.68 mmol), tert-butyl 3-[(2-fluoro-4-iodo-phenyl)methoxy]azetidine-1-carboxylate (1.0 g, 2.46 mmol) and bpy (383.53 mg, 2.46 mmol) in ACN (10 mL), and then was added CuI (467.68 mg, 2.46 mmol) under N₂ atmosphere. The mixture stirred at 100° C. for 17 h under N₂ atmosphere. The mixture was filtered and evaporated. Purification by FC (SiO2; PE/EtOAc) gave the title compound (1.68 g, 90% yield) as a white solid. MS (ESI): m/z=282.2 [M-BOC+H]⁺.

Step 3: tert-Butyl 3-[[2-fluoro-4-(trifluoromethylsulfonyl)phenyl]methoxy]azetidine-1-carboxylate

To a solution of tert-butyl 3-[[2-fluoro-4-(trifluoromethylsulfanyl)phenyl]methoxy]azetidine-1-carboxylate (1.58 g, 4.14 mmol) in 1:1:2 1,2-dichloroethane/ACN/water (60 mL) was added sodium periodate (1.77 g, 8.29 mmol) and ruthenium (III) chloride hydrate (9.34 mg, 0.040 mmol), at 0° C. The mixture was stirred for 12 h at 30° C., before being extracted with EtOAc (3×). The combined organic layers were washed with brine (3×), dried over Na2SO4, filtered, and evaporated. Purification by FC (SiO2; PE/EtOAc) gave the title compound (1.45 g, 85% yield) as a colorless oil. 1H NMR (400 MHz, CHLOROFORM-d) δ=7.90-7.81 (m, 2H), 7.73 (dd, J=1.1, 8.4 Hz, 1H), 4.63 (s, 2H), 4.46-4.35 (m, 1H), 4.20-4.14 (m, 2H), 3.98-3.86 (m, 2H), 1.45 (s, 9H).

Step 4:3-[[2-Fluoro-4-(trifluoromethylsulfonyl)phenyl]methoxy]azetidine; 4-methylbenzenesulfonic Acid

The product was obtained in analogy to BB24, step 5, starting from tert-butyl 3-[[2-fluoro-4-(trifluoromethylsulfonyl)phenyl]methoxy]azetidine-1-carboxylate to get the title compound as a colorless solid. MS (ESI): m/z=314.1 [M+H]⁺.

### BB97

### 7-[[6-(Trifluoromethyl)-3-pyridyl]methyl]-2-azaspiro[3.5]nonane; 4-methylbenzenesulfonic Acid

Step 1:7-[(4,4,5,5-Tetramethyl-1,3,2-dioxaborolan-2-yl)methylene]-2-azaspiro[3.5]nonane-2-carboxylic Acid Tert-Butyl Ester

A solution of 4,4,5,5-tetramethyl-2-[(4,4,5,5-tetramethyl-1,3,2-dioxaborolan-2-yl)methyl]-1,3,2-dioxaborolane (2.5 g, 9.33 mmol, CAS RN 78782-17-9) in THF (30 mL) was cooled to −75° C. 2 M LDA 2 M in THF/heptane/ethylbenzene (4.88 mL, 9.76 mmol, CAS RN 4111-54-0) was added dropwise below −70° C. The RM was stirred at −75° C. for 15 min. A solution of 7-keto-2-azaspiro[3.5]nonane-2-carboxylic acid tert-butyl ester (2.03 g, 8.48 mmol, CAS RN 1363381-22-9) in THF (12 mL) was added below −70° C. The RM was allowed to warm up to RT. The RM was poured on NH4Cl sat. (150 ml). The organic layer was washed with H2O, NaCl sat., dried over Na2SO4 and concentrated in vacuo: 4.2 g yellow oil. The reaction mixture was poured into AcOET and washed with water, NaCl sat. The organic layer was dried over Na2SO4 and concentrated in vacuo. The crude material was purified by flash chromatography (silica gel, 40 g, 0% to 15% AcOEt in heptane) to afford the desired product as white solid (777 mg, 23.95%). MS (ESI): m/z=308.2 [M-C4H8+H]⁺.

Step 2:7-[[6-(Trifluoromethyl)-3-pyridyl]methylene]-2-azaspiro[3.5]nonane-2-carboxylic Acid Tert-Butyl Ester

A solution of 7-[(4,4,5,5-tetramethyl-1,3,2-dioxaborolan-2-yl)methylene]-2-azaspiro[3.5]nonane-2-carboxylic acid tert-butyl ester (767 mg, 2.01 mmol), 5-bromo-2-(trifluoromethyl)pyridine (475.92 mg, 2.11 mmol) and potassium carbonate (554.4 mg, 4.01 mmol) in 1,4-dioxane (12 mL), water (2.4 mL) was degassed with argon for 10 min. 1,1′-bis(diphenylphosphino) ferrocene-palladium (ii) dichloride DCM complex (163.79 mg, 0.201 mmol, 0.100 eq) was added. The mixture was stirred at 80° C. for 3 h. The reaction mixture was poured into AcOET and washed with water, NaCl sat. The organic layer was dried over Na2SO4 and concentrated in vacuo. The crude material was purified by flash chromatography (silica gel, 40 g, 0% to 20% AcOEt in heptane) to afford the desired product (607 mg, 75.18%) as white solid MS (ESI): m/z=327.1 [M-C4H8+H]⁺.

Step 3: tert-Butyl 7-[[6-(trifluoromethyl)-3-pyridyl]methyl]-2-azaspiro[3.5]nonane-2-carboxylate

A solution of 7-[[6-(trifluoromethyl)-3-pyridyl]methylene]-2-azaspiro[3.5]nonane-2-carboxylic acid tert-butyl ester (604 mg, 1.58 mmol) in THF (15 mL) and methanol (45 mL) was degassed with argon for 10 min. Pd/C 10% (168.08 mg, 0.158 mmol) was added. The mixture was stirred under H₂ balloon at RT for 3 h. The reaction mixture was filtered trough sartorius filter, concentrated in vacuo to afford the desired product (618 mg, 96.69%) as brown solid MS (ESI): m/z=329.1 [M-C4H8+H]⁺.

Step 4:7-[[6-(Trifluoromethyl)-3-pyridyl]methyl]-2-azaspiro[3.5]nonane; 4-methylbenzenesulfonic Acid

The product was obtained in analogy to BB24, step 5, starting from tert-butyl 7-[[6-(trifluoromethyl)-3-pyridyl]methyl]-2-azaspiro[3.5]nonane-2-carboxylate to get the title compound as a colorless solid. MS (ESI): m/z=285.2 [M+H]⁺.

### BB98

### 4-[4-(Azetidin-3-yl)phenyl]-1-methyl-3-(trifluoromethyl)pyrazole; 4-methylbenzenesulfonic Acid

Step 1: tert-Butyl 3-[4-[1-methyl-3-(trifluoromethyl)pyrazol-4-yl]phenyl]azetidine-1-carboxylate

A solution of 1-methyl-4-(4,4,5,5-tetramethyl-1,3,2-dioxaborolan-2-yl)-3-(trifluoromethyl)pyrazole (CAS RN: 1218790-53-4; 106.11 mg, 0.384 mmol), 3-(4-bromophenyl)azetidine-1-carboxylic acid tert-butyl ester (CAS RN: 1064194-10-0; 120 mg, 0.384 mmol), potassium carbonate (265.61 mg, 1.92 mmol) and tetrakis (triphenylphosphine)palladium (0) (22.21 mg, 0.019 mmol) in THF (1.81 mL) and Water (0.181 mL) was vigorously stirred under argon at 80° C. for 3 h. The reaction mixture was poured on water (5 mL) and EtOAc (5 mL) and the layers were separated. The aqueous layer was extracted twice with EtOAc (2×5 mL). The organic layers were combined and dried over MgSO4, filtered, treated with silica gel and evaporated. Purification by FC (SiO2; heptane/EtOAc) gave the title compound (0.135 g, 87.4%) as a yellow gum. MS (ESI): m/z=326.2 [M-C4H8+H]⁺.

Step 2:4-[4-(Azetidin-3-yl)phenyl]-1-methyl-3-(trifluoromethyl)pyrazole; 4-methylbenzenesulfonic Acid

The product was obtained in analogy to BB24, step 5, starting from tert-butyl 3-[4-[1-methyl-3-(trifluoromethyl)pyrazol-4-yl]phenyl]azetidine-1-carboxylate to get the title compound as a yellow solid. MS (ESI): m/z=282.2 [M+H]⁺.

### BB99

### N-[[2-Fluoro-4-(trifluoromethyl)phenyl]methyl]azetidin-3-amine; 4-methylbenzenesulfonic Acid

Step 1: tert-Butyl 3-[[2-fluoro-4-(trifluoromethyl)phenyl]methylamino]azetidine-1-carboxylate

To a solution of 2-fluoro-4-(trifluoromethyl)benzaldehyde (1 g, 5.21 mmol, CAS RN 89763-93-9) and 3-aminoazetidine-1-carboxylic acid tert-butyl ester (986.11 mg, 5.73 mmol, CAS RN 193269-78-2) in DCM (10 mL) was added sodium triacetoxy borohydride (1.21 g, 5.73 mmol) and acetic acid (595.96 μL, 10.41 mmol). The mixture was stirred at RT for 4 h. The reaction mixture was poured into AcOET/THF 2:1 and washed with NaHCO₃ sat., water and brine. The organic layer was dried over Na2SO4 and concentrated in vacuo. The crude material was purified by flash chromatography (silica gel, 40 g, 0% to 5% methanol in DCM) to get the desired product (1.24 g, 64.97%) as colorless oil MS (ESI): m/z=293.1 [M-C4H8+H]⁺.

Step 2: N-[[2-Fluoro-4-(trifluoromethyl)phenyl]methyl]azetidin-3-amine; 4-methylbenzenesulfonic Acid

The product was obtained in analogy to BB24, step 5, starting from tert-butyl 3-[[2-fluoro-4-(trifluoromethyl)phenyl]methylamino]azetidine-1-carboxylate to get the title compound as a colorless solid. MS (ESI): m/z=249.1 [M+H]⁺.

### BB100

### N-[2-Fluoro-4-(trifluoromethyl)phenyl]-2-azaspiro[3.3]heptan-6-amine; 4-methylbenzenesulfonic Acid

Step 1: tert-Butyl 6-[2-fluoro-4-(trifluoromethyl)anilino]-2-azaspiro[3.3]heptane-2-carboxylate

To a solution of 6-amino-2-azaspiro[3.3]heptane-2-carboxylic acid tert-butyl ester (1 g, 4.71 mmol, CAS RN 1211586-09-2) and DIEA (1.61 mL, 9.42 mmol) in DMSO (6 mL) was added 1,2-difluoro-4-(trifluoromethyl)benzene (943.52 mg, 5.18 mmol, CAS RN 32137-19-2). The mixture was stirred at 100° C. for 1 h. The reaction mixture was poured into AcOET and washed with water and brine. The organic layer was dried over Na2SO4 and concentrated in vacuo: 0.96 g yellow oil. The crude material was purified by flash chromatography (silica gel, 40 g, 0% to 80% AcOEt in heptane) to afford the desired product (33 mg, 1.78%) as white solid. MS (ESI): m/z=375.4 [M+H]⁺.

Step 2: N-[2-Fluoro-4-(trifluoromethyl)phenyl]-2-azaspiro[3.3]heptan-6-amine; 4-methylbenzenesulfonic Acid

The product was obtained in analogy to BB24, step 5, starting from tert-butyl 6-[2-fluoro-4-(trifluoromethyl)anilino]-2-azaspiro[3.3]heptane-2-carboxylate to get the title compound as a colorless solid. MS (ESI): m/z=275.1 [M+H]⁺.

### BB101

### 7-[5-(Trifluoromethyl)pyrimidin-2-yl]oxy-2-azaspiro[3.5]nonane; 4-methylbenzenesulfonic Acid

Step 1: tert-Butyl 7-[5-(trifluoromethyl)pyrimidin-2-yl]oxy-2-azaspiro[3.5]nonane-2-carboxylate

To a solution of 7-hydroxy-2-azaspiro[3.5]nonane-2-carboxylic acid tert-butyl ester (0.500 g, 2.07 mmol, CAS RN 1363383-18-9) and potassium tert-butoxide (278.98 mg, 2.49 mmol) in DMSO, extra dry (3 mL) was added 2-chloro-5-(trifluoromethyl)pyrimidine (378.18 mg, 2.07 mmol, CAS RN 69034-12-4). The mixture was stirred at RT for 1 h. The reaction mixture was poured into AcOET and washed with water and brine. The organic layer was dried over Na2SO4 and concentrated in vacuo. The crude material was purified by flash chromatography (silica gel, 12 g, 0% to 50% AcOEt in heptane) to afford the desired product as a white solid (198 mg, 19.7%). MS (ESI): m/z=332.1 [M-C4H8+H]⁺.

Step 2:7-[5-(Trifluoromethyl)pyrimidin-2-yl]oxy-2-azaspiro[3.5]nonane; 4-methylbenzenesulfonic Acid

The product was obtained in analogy to BB24, step 5, starting from tert-butyl 7-[5-(trifluoromethyl)pyrimidin-2-yl]oxy-2-azaspiro[3.5]nonane-2-carboxylate to get the title compound as a colorless solid. MS (ESI): m/z=288.1 [M+H]⁺.

### BB102

### 7-[5-(Trifluoromethyl)pyrazin-2-yl]oxy-2-azaspiro[3.5]nonane; 4-methylbenzenesulfonic Acid

Step 1: tert-Butyl 7-[5-(trifluoromethyl)pyrazin-2-yl]oxy-2-azaspiro[3.5]nonane-2-carboxylate

To a solution of 7-hydroxy-2-azaspiro[3.5]nonane-2-carboxylic acid tert-butyl ester (1.39 g, 5.76 mmol, CAS RN 1363383-18-9) and potassium t-butoxide (775.58 mg, 6.91 mmol) in DMF (13.9 mL) was added 2-fluoro-5-(trifluoromethyl)pyrazine (1. g, 6.05 mmol, CAS RN 1220799-65-4). The mixture was stirred at 80° C. for 15 h. The reaction mixture was poured into AcOET and washed with water and brine. The organic layer was dried over Na2SO4 and concentrated in vacuo. The crude material was purified by flash chromatography (silica gel, 50 g, 0% to 15% AcOEt in heptane) to afford the desired product as a white solid (902 mg, 38.4%). MS (ESI): m/z=332.1 [M-C4H8+H]⁺.

Step 2:7-[5-(Trifluoromethyl)pyrazin-2-yl]oxy-2-azaspiro[3.5]nonane; 4-methylbenzenesulfonic Acid

The product was obtained in analogy to BB24, step 5, starting from tert-butyl 7-[5-(trifluoromethyl)pyrazin-2-yl]oxy-2-azaspiro[3.5]nonane-2-carboxylate to get the title compound as a colorless solid. MS (ESI): m/z=288.1 [M+H]⁺.

### BB103

### 2-[[4-Fluoro-2-(trifluoromethyl)phenyl]methyl]-2,6-diazaspiro[3.3]heptane; trifluoroacetic Acid

Step 1: tert-Butyl 6-(4-fluoro-2-(trifluoromethyl)benzoyl)-2,6-diazaspiro[3.3]heptane-2-carboxylate

To a solution of tert-butyl 2,6-diazaspiro[3.3]heptane-2-carboxylate (400 mg, 2.02 mmol) in CH2Cl2 (9 mL) cooled down to 0° C. was added DIPEA (652 mg, 881 μL, 5.04 mmol) and 4-fluoro-2-(trifluoromethyl)benzoyl chloride (503 mg, 2.22 mmol). The reaction mixture was stirred at 0° C. for 10 min and at RT for 18 h. The reaction mixture was diluted with dichloromethane and extracted with aq. Na₂CO3 1 M solution, the organic phase was collected and the aqueous phase was back-extracted with dichloromethane. The combined organic phases were dried over sodium sulfate and evaporated down to dryness. The crude material was purified by flash chromatography, using an eluent mixture of heptane and EtOAc (5% to 80%) to give the title compound (569 mg). MS (ESI): m/z=389.3 [M+H]⁺.

Step 2: tert-Butyl 6-(4-fluoro-2-(trifluoromethyl)benzyl)-2,6-diazaspiro[3.3]heptane-2-carboxylate

To a solution of tert-butyl 6-(4-fluoro-2-(trifluoromethyl)benzoyl)-2,6-diazaspiro[3.3]heptane-2-carboxylate (565 mg, 1.45 mmol) in dry THF (5 mL) was slowly added borane THF complex 1.0 M (3.64 mL, 3.64 mmol) and the reaction mixture was then refluxed for 20 h. The reaction was cooled down to 0° C. followed by addition of slow addition of methanol to quench excess borane after which it was stirred at 23° C. for 15 min followed by stirring at 55° C. for 18 h. Volatiles were removed in vacuo and the crude residue was directly purified by flash chromatography using an eluent mixture of dichloromethane and methanol (0% to 10%) to yield 417 mg of the title compound. MS (ESI): m/z=375.2 [M+H]⁺.

Step 3:2-[[4-Fluoro-2-(trifluoromethyl)phenyl]methyl]-2,6-diazaspiro[3.3]heptane; trifluoroacetic Acid

To a solution of tert-butyl 6-(4-fluoro-2-(trifluoromethyl)benzyl)-2,6-diazaspiro[3.3]heptane-2-carboxylate (455 mg, 1.09 mmol) in dichloromethane (4 mL) was added TFA (843 μL, 10.9 μL) and the reaction mixture was stirred at RT for 18 h. Volatiles were removed in vacuo to yield 685 mg of the crude title compound (purity roughly 80%) which was used without further purification. MS (ESI): m/z=275.2 [M+H]⁺.

### BB104

### 4-(Azetidin-3-yl)-N-[1-(trifluoromethyl)cyclopropyl]aniline; 4-methylbenzenesulfonic Acid

Step 1: tert-Butyl 3-(4-bromophenyl)azetidine-1-carboxylate

To a stirred suspension of 4-bromophenylboronic acid (7093.71 mg, 35.32 mmol) in 2-propanol (50 mL) was added a solution of 1-BOC-3-iodoazetidine (5000.0 mg, 17.66 mmol) in 2-propanol (25 mL) at RT. To the mixture was added rac-(1R,2R)-2-aminocyclohexan-1-ol (122.04 mg, 1.06 mmol), nickel (II) iodide (331.14 mg, 1.06 mmol) and sodium bis(trimethylsilyl)amide in THF (35.32 mL, 35.32 mmol) under nitrogen. Afterwards the resulting mixture was stirred at 20° C. for 30 minutes, then the mixture was divided into 10 sealed tube and heated under microwave irradiation at 80° C. for 30 minutes. The reaction mixture was poured onto a mixture of H₂O and EtOAc and the layers were separated. The aqueous layer was back-extracted twice with EtOAc. The combined organic layers were dried over Na2SO4, filtered, and concentrated, the obtained residue was purified by prep-HPLC (0.225% v/vFA) and concentrated to give the title compound (3000.0 mg, 9.61 mmol, 54.41% yield) as light yellow oil. MS (ESI): m/z=256.1 [M-C4H8+H]⁺.

Step 2: tert-Butyl 3-[4-[[1-(trifluoromethyl)cyclopropyl]amino]phenyl]azetidine-1-carboxylate

To a solution of tert-butyl 3-(4-bromophenyl)azetidine-1-carboxylate (500.0 mg, 1.6 mmol), 1-(trifluoromethyl)cyclopropanamine hydrochloride (388.09 mg, 2.4 mmol) and sodium tert-butoxide (615.65 mg, 6.41 mmol) in t-Amyl-OH (10.0 mL) was added tBuXPhos-Pd-G3 (127.22 mg, 0.16 mmol), the mixture was then stirred under nitrogen atmosphere at 110° C. for 12 hours. The reaction mixture was poured into water (20 mL) and extracted with EtOAc (10 mL three times), the combined organic layers were washed with brine then dried over Na2SO4. The solution was concentrated and the crude product was purified by column chromatography on silica gel eluted with petroleum:EtOAc=10:1 to 5:1 then reverse flash (0.1% v/v FA) to give the title compound (100.0 mg, 0.28 mmol, 17.52% yield) as light yellow oil. MS (ESI): m/z=301.4 [M-C4H8+H]⁺.

Step 3:4-(Azetidin-3-yl)-N-[1-(trifluoromethyl)cyclopropyl]aniline; 4-methylbenzenesulfonic Acid

The product was obtained in analogy to BB24, step 5, starting from tert-butyl 3-[4-[[1-(trifluoromethyl)cyclopropyl]amino]phenyl]azetidine-1-carboxylate to get the title compound as a grey solid. MS (ESI): m/z=257.4 [M+H]⁺.

### BB105

### 7-[6-(Trifluoromethyl)pyridazin-3-yl]oxy-2-azaspiro[3.5]nonane; 4-methylbenzenesulfonic Acid

Step 1:7-[6-(Trifluoromethyl)pyridazin-3-yl]oxy-2-azaspiro[3.5]nonane-2-carboxylic acid tert-butyl Ester

To a solution of 7-hydroxy-2-azaspiro[3.5]nonane-2-carboxylic acid tert-butyl ester (CAS: 1363383-18-9) (350 mg, 1.45 mmol) and potassium t-butoxide (195 mg, 1.74 mmol) in N,N-dimethylformamide (3.5 mL) was added 3-fluoro-6-(trifluoromethyl)pyridazine (248 mg, 1.49 mmol). The mixture was stirred at 80° C. for 15 h. The reaction mixture was poured into EtOAc and washed with water and brine. The organic layer was dried over Na2SO4 and concentrated in vacuo. The crude material was purified by flash chromatography (eluting with 0% to 40% EtOAc in heptane) to afford the title compound (353 mg, 59.69%) as white solid. MS (ESI): m/z=332.1 [M-C4H8+H]⁺.

Step 2:7-[6-(Trifluoromethyl)pyridazin-3-yl]oxy-2-azaspiro[3.5]nonane; 4-methylbenzenesulfonic Acid

To a suspension of 7-[6-(trifluoromethyl)pyridazin-3-yl]oxy-2-azaspiro[3.5]nonane-2-carboxylic acid tert-butyl ester (350 mg, 0.903 mmol) in isopropyl acetate (4 mL) and was added p-toluenesulfonic acid monohydrate (258 mg, 1.36 mmol). The mixture was stirred at 100° C. for 6 h. The reaction mixture was concentrated in vacuo. Et20 was added, and the mixture filtered through sintered glass. The white solid was washed with Et20 (2×) and dried in vacuo to afford the title compound (0.420 g, 88%) as a white solid. MS (ESI): m/z=288.0 [M+H]⁺.

### BB106

### 6-[[3-Fluoro-5-(trifluoromethyl)-2-pyridyl]methyl]-2-azaspiro[3.3]heptane; 4-methylbenzenesulfonic Acid

Step 1: tert-Butyl 6-[(4,4,5,5-tetramethyl-1,3,2-dioxaborolan-2-yl)methylene]-2-azaspiro[3.3]heptane-2-carboxylate

To a solution of 2,2,6,6-tetramethylpiperidine (3.3 mL, 19.54 mmol) in THF (30 mL) was added 1.6 M BuLi (12.21 mL, 19.54 mmol) between −45° C. and −35° C. The mixture was stirred and allowed to warm up to 5° C. The reaction mixture was cooled to −74° C. A solution of 4,4,5,5-tetramethyl-2-[(4,4,5,5-tetramethyl-1,3,2-dioxaborolan-2-yl)methyl]-1,3,2-dioxaborolane (4.8 g, 17.91 mmol) in THF (15 mL) was added dropwise below −70° C. The reaction mixture was stirred at −74° C. for 30 minutes. A solution of 6-keto-2-azaspiro[3.3]heptane-2-carboxylic acid tert-butyl ester (3.44 g, 16.28 mmol) in THF (22 mL) was added dropwise below −70° C. in 45 minutes. The reaction mixture was allowed to warm up to room temperature and stirred at room temperature for 3 hours. The reaction mixture was poured on saturated aqueous NH4Cl solution (210 ml). Aqueous HCl 2 M solution was added to adjust the pH to 6. The mixture was extracted with EtOAc. The organic layer was washed with H2O, brine, dried over Na2SO4 and concentrated in vacuo to give 7.2 g of the crude title compound as a yellow solid. The crude material was purified by flash chromatography (silica gel, 80 g, 0% to 20% AcOEt in heptane) to afford the title compound (4.37 g, 76.05%) as white solid. MS (ESI): m/z=279.9 [M-C4H8+H]⁺.

Step 2: tert-Butyl 6-[[3-fluoro-5-(trifluoromethyl)-2-pyridyl]methylene]-2-azaspiro[3.3]heptane-2-carboxylate

To a solution of 2-bromo-3-fluoro-5-(trifluoromethyl)pyridine (1.46 g, 5.97 mmol) and tert-butyl 6-[(4,4,5,5-tetramethyl-1,3,2-dioxaborolan-2-yl)methylene]-2-azaspiro[3.3]heptane-2-carboxylate (2.0 g, 5.97 mmol), potassium carbonate (1648.99 mg, 11.93 mmol, 2.0 eq) in 1,4-Dioxane (20 mL) and water (4 mL) was added 1,1′-bis(diphenylphosphino) ferrocene-palladium(II) dichloride dichloromethane complex (486.8 mg, 0.6 mmol), the mixture was degassed with nitrogen and stirred at 80° C. under nitrogen atmosphere for 12 hours. The reaction mixture was concentrated under reduced pressure to give a residue. The residue was purified by flash silica gel chromatography (Eluent of 0~8% EtOAc/Petroleum ether) to give the title compound (1.8 g, 4.83 mmol, 81.03% yield) as a white solid confirmed MS (ESI): m/z=317.2 [M-C4H8+H]⁺.

Step 3: tert-Butyl 6-[[3-fluoro-5-(trifluoromethyl)-2-pyridyl]methyl]-2-azaspiro[3.3]heptane-2-carboxylate

To a solution of tert-butyl 6-[[3-fluoro-5-(trifluoromethyl)-2-pyridyl]methylene]-2-azaspiro[3.3]heptane-2-carboxylate (1.7 g, 4.57 mmol) in EtOAc (30 mL) was added Pd/C (700.0 mg) under nitrogen. The suspension was degassed under vacuum and purged with hydrogen several times. The mixture was stirred under hydrogen atmosphere at 25° C. for 2 hours. The reaction mixture was filter and the filtrated was concentrated to give the title compound (1.7 g, 4.54 mmol, 99.46% yield) as a white solid. MS (ESI): m/z=319.2 [M-C4H8+H]⁺.

Step 4: tert-Butyl 6-[[3-fluoro-5-(trifluoromethyl)-2-pyridyl]methyl]-2-azaspiro[3.3]heptane-2-carboxylate

The mixture of tert-butyl 6-[[3-fluoro-5-(trifluoromethyl)-2-pyridyl]methyl]-2-azaspiro[3.3]heptane-2-carboxylate (1.7 g, 4.54 mmol) and TsOH (1640.39 mg, 9.54 mmol) in EtOAc (20 mL) was stirred at 80° C. for 16 hours. The residue was concentrated under reduced pressure to give the title compound (2.52 g, 4.07 mmol, 89.07% yield) as yellow solid. MS (ESI): m/z=275.2 [M+H]⁺.

### BB107

### 6-[[4-Fluoro-2-(trifluoromethyl)phenyl]methyl]-2-azaspiro[3.3]heptane; 4-methylbenzenesulfonic Acid

Step 1: tert-Butyl 6-[(4,4,5,5-tetramethyl-1,3,2-dioxaborolan-2-yl)methylene]-2-azaspiro[3.3]heptane-2-carboxylate

To a solution of 2,2,6,6-tetramethylpiperidine (2.76 g, 3.3 mL, 19.54 mmol) in THF (30 mL) was added 1.6 M BuLi (12.21 mL, 19.54 mmol) between −45° C. and −35° C. The mixture was stirred and allowed to warm up to 5° C. The reaction mixture was cooled to −74° C. A solution of 4,4,5,5-tetramethyl-2-[(4,4,5,5-tetramethyl-1,3,2-dioxaborolan-2-yl)methyl]-1,3,2-dioxaborolane (4.8 g, 17.91 mmol) in THF (15 mL) was added dropwise below −70° C. The reaction mixture was stirred at −74° C. for 30 minutes. A solution of 6-keto-2-azaspiro[3.3]heptane-2-carboxylic acid tert-butyl ester (3.44 g, 16.28 mmol) in THF (22 mL) was added dropwise below −70° C. in 45 minutes. The reaction mixture was allowed to warm up to room temperature and stirred at room temperature for 3 hours. The reaction mixture was poured on saturated aqueous NH₄Cl solution (210 ml). Aqueous HCl 2 M solution was added to adjust the pH to 6. The mixture was extracted with EtOAc. The organic layer was washed with H2O, brine, dried over Na2SO4 and concentrated in vacuo to give 7.2 g of the crude title compound as a yellow solid. The crude material was purified by flash chromatography (silica gel, 80 g, 0% to 20% AcOEt in heptane) to afford the title compound (4.37 g, 76.05%) as white solid. MS (ESI): m/z=279.9 [M-C4H8+H]⁺.

Step 2: tert-Butyl 6-[[4-fluoro-2-(trifluoromethyl)phenyl]methylene]-2-azaspiro[3.3]heptane-2-carboxylate

2-bromo-5-fluorobenzotrifluoride (2.96 g, 12.17 mmol), tert-butyl 6-[(4,4,5,5-tetramethyl-1,3,2-dioxaborolan-2-yl)methylene]-2-azaspiro[3.3]heptane-2-carboxylate (3.4 g, 10.14 mmol), 1,1′-bis(diphenylphosphino) ferrocene-palladium(II) dichloride dichloromethane complex (1.66 g, 2.03 mmol) and potassium carbonate (2.8 g, 20.28 mmol) were dissolved in 1,4-Dioxane (51 mL) and water (10 mL). The reaction mixture was stirred at 120° C. under argon for 16 hours. The reaction mixture was concentrated under reduced pressure. The residue was partitioned between EtOAc and water. The organic layer was washed with brine. The extract was dried over sodium sulfate, filtered through a thin layer of silica gel and evaporated. The crude product was purified by column chromatography (120 g SiO2, hexane/MTBE (0-20-100%), flow rate=85 ml/min) to afford the title compound (2.0 g, 5.39 mmol, 50.45% yield) as light yellow solid. MS (ESI): m/z=317.0 [M-C4H8+H]⁺.

Step 3: tert-Butyl 6-[[4-fluoro-2-(trifluoromethyl)phenyl]methyl]-2-azaspiro[3.3]heptane-2-carboxylate

A mixture of tert-butyl 6-[[4-fluoro-2-(trifluoromethyl)phenyl]methylene]-2-azaspiro[3.3]heptane-2-carboxylate (2.0 g, 5.39 mmol) and platinum (0.2 g, 0.1 mmol) in EtOAc (40 mL) was stirred in an autoclave for 24 h under 40 bar of H2. Then the reaction mixture was filtered through a thin layer of silica gel, and concentrated to afford the title compound (1.8 g, 4.82 mmol, 85.04% yield) as light yellow solid. Used as is for next step.

Step 4:6-[[4-Fluoro-2-(trifluoromethyl)phenyl]methyl]-2-azaspiro[3.3]heptane; 4-methylbenzenesulfonic Acid

A mixture of p-toluenesulfonic acid (1.0 g, 5.78 mmol) and tert-butyl 6-[[4-fluoro-2-(trifluoromethyl)phenyl]methyl]-2-azaspiro[3.3]heptane-2-carboxylate (1.8 g, 4.82 mmol) in EtOAc (40 mL) was stirred at 50° C. for 24 hours. After the completion of the reaction, the reaction mixture was concentrated and 20 mL of cold Et20 was added. The crystals that formed were filtered, washed with MTBE and dried to afford the title compound (1.75 g, 3.93 mmol, 77.42% yield) as white solid. MS (ESI): m/z=274.0 [M+H]⁺.

### BB108

### [3-(2-Azaspiro[3.3]heptan-6-ylmethyl)phenyl]-imino-oxo-(trifluoromethyl)-λ²-sulfane; 4-methylbenzenesulfonic Acid

Step 1: tert-Butyl 6-[(4,4,5,5-tetramethyl-1,3,2-dioxaborolan-2-yl)methylene]-2-azaspiro[3.3]heptane-2-carboxylate

To a solution of 2,2,6,6-tetramethylpiperidine (2.76 g, 3.3 mL, 19.54 mmol) in THF (30 mL) was added 1.6 M BuLi (12.21 mL, 19.54 mmol) between −45° C. and −35° C. The mixture was stirred and allowed to warm up to 5° C. The reaction mixture was cooled to −74° C. A solution of 4,4,5,5-tetramethyl-2-[(4,4,5,5-tetramethyl-1,3,2-dioxaborolan-2-yl)methyl]-1,3,2-dioxaborolane (4.8 g, 17.91 mmol) in THF (15 mL) was added dropwise below −70° C. The reaction mixture was stirred at −74° C. for 30 minutes. A solution of 6-keto-2-azaspiro[3.3]heptane-2-carboxylic acid tert-butyl ester (3.44 g, 16.28 mmol) in THF (22 mL) was added dropwise below −70° C. in 45 minutes. The reaction mixture was allowed to warm up to room temperature and stirred at room temperature for 3 hours. The reaction mixture was poured on saturated aqueous NH₄Cl solution (210 ml). Aqueous HCl 2 M solution was added to adjust the pH to 6. The mixture was extracted with EtOAc. The organic layer was washed with H2O, brine, dried over Na2SO4 and concentrated in vacuo to give 7.2 g of the crude title compound as a yellow solid. The crude material was purified by flash chromatography (silica gel, 80 g, 0% to 20% AcOEt in heptane) to afford the title compound (4.37 g, 76.05%) as white solid. MS (ESI): m/z=279.9 [M-C4H8+H]⁺.

Step 2: tert-Butyl 6-[[3-(trifluoromethylsulfonimidoyl)phenyl]methylene]-2-azaspiro[3.3]heptane-2-carboxylate (3-bromophenyl)-imino-oxo-(trifluoromethyl)-26-sulfane (2.47 g, 8.59 mmol), tert-butyl 6-[(4,4,5,5-tetramethyl-1,3,2-dioxaborolan-2-yl)methylene]-2-azaspiro[3.3]heptane-2-carboxylate (2.4 g, 7.16 mmol), 1,1′-bis(diphenylphosphino) ferrocene-palladium(II) dichloride dichloromethane complex (1.17 g, 1.43 mmol) and potassium carbonate (1.98 g, 14.3 mmol) were dissolved in 1,4-Dioxane (40 mL) and water (8 mL). The reaction mixture was heated to 120° C. under argon for 16 h. The reaction mixture was concentrated under reduced pressure. The residue was partitioned between EtOAc and water. The organic layer was washed with brine. The extract was dried over sodium sulfate, filtered through a thin layer of silica gel and evaporated. The crude product was purified by column chromatography to afford the title compound (1 g, 2.4 mmol, 31.9% yield) as light yellow solid. MS (ESI): m/z=361.0 [M-C4H8+H]⁺.
Step 3: tert-Butyl 6-[[3-(trifluoromethylsulfonimidoyl)phenyl]methyl]-2-azaspiro[3.3]heptane-2-carboxylate

A mixture of tert-butyl 6-[[3-(trifluoromethylsulfonimidoyl)phenyl]methylene]-2-azaspiro[3.3]heptane-2-carboxylate (1.3 g, 3.12 mmol) and palladium on carbon (10%) (0.16 mL, 1.56 mmol) in EtOAc (35 mL) was stirred in an autoclave for 24 h under 30 bar of H2. Then the reaction mixture was filtered and concentrated to afford the title compound (1.3 g, 3.11 mmol, 96.5% yield) a as grey oil. MS (ESI): m/z=319.0 [M-BOC+H]⁺.

Step 4: [3-(2-Azaspiro[3.3]heptan-6-ylmethyl)phenyl]-imino-oxo-(trifluoromethyl)-26-sulfane; 4-methylbenzenesulfonic Acid

A mixture of p-toluenesulfonic acid (1.18 g, 6.83 mmol) and tert-butyl 6-[[3-(trifluoromethylsulfonimidoyl)phenyl]methyl]-2-azaspiro[3.3]heptane-2-carboxylate (1.3 g, 3.11 mmol) in EtOAc (30 mL) was stirred at 40° C. for 24 h. After the completion of the reaction, the reaction mixture was concentrated and purified by HPLC to afford the title compound (339 mg, 0.690 mmol, 15.6% yield) as brown viscous oil. MS (ESI): m/z=319.0 [M+H]⁺.

### BB109

### 2-[[3-(Azetidin-3-yl)-1-bicyclo[1.1.1]pentanyl]oxy]-5-(trifluoromethyl)pyrazine; 4-methylbenzenesulfonic Acid

Step 1: tert-Butyl 3-[3-[5-(trifluoromethyl)pyrazin-2-yl]oxy-1-bicyclo[1.1.1]pentanyl]azetidine-1-carboxylate

A solution of tert-butyl 3-(3-hydroxy-1-bicyclo[1.1.1]pentanyl)azetidine-1-carboxylate (CAS RN: 2490406-07-8; 700.0 mg, 2.93 mmol) and 2-fluoro-5-(trifluoromethyl)pyrazine (CAS RN: 69045-82-5; 534.38 mg, 3.22 mmol) in ACN (30 mL) was treated with cesium carbonate (1906.1 mg, 5.85 mmol), at 25° C. under Ar. The mixture was stirred at 80° C. for 16 h, before being filtered. The cake was washed with ACN (30 mL), and the filtrate was evaporated. The residue was diluted with H₂O (30 mL) and extracted with EtOAc (3×10 mL). The combined organic layers were washed with saturated aqueous NaCl, dried over Na2SO4, and evaporated, to give the title compound (200 mg, 42.14% yield) as a light brown solid. MS (ESI): m/z=286 [M-BOC+H]⁺.

Step 2:2-[[3-(Azetidin-3-yl)-1-bicyclo[1.1.1]pentanyl]oxy]-5-(trifluoromethyl)pyrazine; 4-methylbenzenesulfonic Acid

A solution of tert-butyl 3-[3-[5-(trifluoromethyl)pyrazin-2-yl]oxy-1-bicyclo[1.1.1]pentanyl]azetidine-1-carboxylate (200.0 mg, 0.52 mmol) in EtOAc (25 mL) was treated with PTSA (148.08 mg, 0.78 mmol), at 23° C. The mixture was stirred for 16 h at this temperature before being cooled down to 0° C. and stirred for 1 h at that temperature. The resulting precipitate was collected by filtration and washed twice with MTBE (2*50 mL), to give the title compound (190.0 mg, 76.03% yield) as a yellow solid. MS (ESI): m/z=286.2 [M+H]⁺.

### BB110

### 4-Methylbenzenesulfonic acid; (6S or 6R)-6-[[3-(trifluoromethylsulfonyl)phenyl]methyl]-2-azaspiro[3.4]octane

Step 1: tert-Butyl (6E)-6-[(4,4,5,5-tetramethyl-1,3,2-dioxaborolan-2-yl)methylene]-2-azaspiro[3.4]octane-2-carboxylate

2,2,6,6-Tetramethylpiperidine (2.34 mL, 13.85 mmol, 1.2 eq) was dissolved in THF (15 ml) and cooled to −30° C. under an argon atmosphere. butyllithium solution (2.5 M) (5.54 mL, 13.85 mmol, 1.2 eq) was added dropwise, and the reaction mixture was stirred at the same temperature for 30 min. Next, the reaction was cooled to −78° C., and a solution of 4,4,5,5-tetramethyl-2-[(4,4,5,5-tetramethyl-1,3,2-dioxaborolan-2-yl)methyl]-1,3,2-dioxaborolane (3401.96 mg, 12.7 mmol, 1.1 eq) in THF (10 ml) was added dropwise. After stirring for 30 min, a solution of the tert-butyl (6E)-6-[(4,4,5,5-tetramethyl-1,3,2-dioxaborolan-2-yl)methylene]-2-azaspiro[3.4]octane-2-carboxylate (2.7 g, 7.73 mmol) in THF (20 ml) was added dropwise at −78° C. The reaction mixture was allowed to slowly warm up to room temperature and stirred overnight. The obtained mixture was then cooled to 0° C. and saturated aqueous NH₄Cl solution (50 mL) was added dropwise. After additional stirring for 1 h, the resulting mixture was filtered. Water (50 mL) was added to the obtained filtrate, and mixture was extracted with EtOAc (250 mL). The organic phase was washed with brine (200 mL), dried with the anhydrous Na₂SO4 and concentrated under vacuum. The obtained crude product was purified by column chromatography (Interchim, 120 g SiO2, hexane/MTBE (0-15%), flow rate=85 ml/min, 5.5-9 CV) to obtain the title compound (2.7 g, 7.73 mmol, 63.63% yield) as light yellow solid. MS (ESI): m/z=294.4 [M-BOC+H]⁺.

Step 2: tert-Butyl (6E)-6-[[3-(trifluoromethylsulfonyl)phenyl]methylene]-2-azaspiro[3.4]octane-2-carboxylate

To a solution of 1-bromo-3-(trifluoromethylsulfonyl)benzene (CAS: 2728-70-3) (827.64 mg, 2.86 mmol, 1.0 eq) and tert-butyl (6E)-6-[(4,4,5,5-tetramethyl-1,3,2-dioxaborolan-2-yl)methylene]-2-azaspiro[3.4]octane-2-carboxylate (1000.0 mg, 2.86 mmol, 1.0 eq), potassium carbonate (791.4 mg, 5.73 mmol, 2.0 eq) in 1,4-Dioxane (10 mL) and water (2 mL) was added 1,1′-bis(diphenylphosphino) ferrocene-palladium(II) dichloride dichloromethane complex (233.63 mg, 0.29 mmol, 0.1 eq), the mixture was degassed with nitrogen and stirred at 80° C. under nitrogen atmosphere for 12 hours. The mixture was partitioned between EtOAc and water. The organic phase was washed with brine, dried over anhydrous Na2SO4, concentrated in vacuum to give a residue, which was purified by flash silica gel chromatography (Silica Flash Column, Eluent of 0~20% EtOAc/Petroleum ether gradient) to afford the pure title compound (1.23 g, 2.85 mmol, 99.57% yield) as yellow oil. MS (ESI): m/z=376.1, [M-C4H8+H]⁺.

Step 3: tert-Butyl 6-[[3-(trifluoromethylsulfonyl)phenyl]methyl]-2-azaspiro[3.4]octane-2-carboxylate

To a solution of tert-butyl (6E)-6-[3-(trifluoromethylsulfonyl)phenyl]methylene]-2-azaspiro[3.4]octane-2-carboxylate (1.2 g, 2.78 mmol, 1.0 eq) in EtOAc (12 mL) was added wet Pd/C (400.0 mg, 0.28 mmol, 0.1 eq) under nitrogen atmosphere, the mixture was stirred at 25° C. under H₂ atmosphere (balloon) for 2 hours. The mixture was then filtered and the filtrate was concentrated to give a crude residue. The residue was purified by flash silica gel chromatography (Silica Flash Column, Eluent of 0~20% EtOAc/Petroleum ether gradient) to give a crude product. The residue was purified by prep-HPLC (Phenomenex Luna C8 250*50 mm*10 μm, water (FA)-ACN, 20 min, 100 ml/min) to the title compound (1.2 g, 2.77 mmol, 99.54% yield) as a colorless oil. MS (ESI): m/z=378.1 [M-C4H8+H]⁺.

Step 4: tert-Butyl (6S or 6R)-6-[[3-(trifluoromethylsulfonyl)phenyl]methyl]-2-azaspiro[3.4]octane-2-carboxylate

The tert-butyl 6-[[3-(trifluoromethylsulfonyl)phenyl]methyl]-2-azaspiro[3.4]octane-2-carboxylate (1.2 g, 2.77 mmol, 1.0 eq) was further separated by SFC (condition: Column: Chiralpak IG-3 50×4.6 mm I.D., 3 μm Mobile phase: Phase A for CO2, and Phase B for EtOH (0.05% DEA); Gradient elution:EtOH (0.05% DEA) in CO2 from 5% to 10% Flow rate: 3 mL/min; Detector: PDA Column Temp: 35° C.; Back Pressure: 100 Bar) to give tert-butyl (6R or 6S)-6-[3-(trifluoromethylsulfonyl)phenyl]methyl]-2-azaspiro[3.4]octane-2-carboxylate (490.0 mg, 1.13 mmol, 40.83% yield) (ee=96.168%, Optical rotation: +10.524°) as yellow oil and tert-butyl (6S or 6R)-6-[3-(trifluoromethylsulfonyl)phenyl]methyl]-2-azaspiro[3.4]octane-2-carboxylate (ee=100%, Optical rotation: −13.342° (290.0 mg, 0.67 mmol, 24.17% yield) as yellow oil.

(The absolute configuration of the stereocenter of each enantiomer was set arbitrarily)

Step 5:4-Methylbenzenesulfonic acid; (6S)-6-[[3-(trifluoromethylsulfonyl)phenyl]methyl]-2-azaspiro[3.4]octane

The mixture of tert-butyl (6S)-6-[[3-(trifluoromethylsulfonyl)phenyl]methyl]-2-azaspiro[3.4]octane-2-carboxylate (310.0 mg, 0.72 mmol, 1.0 eq) and TsOH (147.62 mg, 0.86 mmol, 1.2 eq) in EtOAc (3 mL) was stirred at 80° C. for 16 hours. The residue was concentrated under reduced pressure to give the title compound (354.26 mg, 0.7 mmol, 97.3% yield) as white solid. MS (ESI): m/z=334.1 [M+H]⁺.

### BB111

### 4-Methylbenzenesulfonic acid; (6S)-6-[[3-(trifluoromethylsulfonyl)phenyl]methyl]-2-azaspiro[3.4]octane

Step 1: tert-Butyl 6-[(4,4,5,5-tetramethyl-1,3,2-dioxaborolan-2-yl)methylene]-2-azaspiro[3.3]heptane-2-carboxylate

To a solution of 2,2,6,6-tetramethylpiperidine (3.3 mL, 19.54 mmol) in THF (30 mL) was added 1.6 M BuLi (12.21 mL, 19.54 mmol) between −45° C. and −35° C. The mixture was stirred and allowed to warm up to 5° C. The reaction mixture was cooled to −74° C. A solution of 4,4,5,5-tetramethyl-2-[(4,4,5,5-tetramethyl-1,3,2-dioxaborolan-2-yl)methyl]-1,3,2-dioxaborolane (4.8 g, 17.91 mmol) in THF (15 mL) was added dropwise below −70° C. The reaction mixture was stirred at −74° C. for 30 minutes. A solution of 6-keto-2-azaspiro[3.3]heptane-2-carboxylic acid tert-butyl ester (3.44 g, 16.28 mmol) in THF (22 mL) was added dropwise below −70° C. in 45 minutes. The reaction mixture was allowed to warm up to room temperature and stirred at room temperature for 3 hours. The reaction mixture was poured on saturated aqueous NH4Cl solution (210 ml). Aqueous HCl 2 M solution was added to adjust the pH to 6. The mixture was extracted with EtOAc. The organic layer was washed with H₂O, brine, dried over Na2SO4 and concentrated in vacuo to give 7.2 g of the crude title compound as a yellow solid. The crude material was purified by flash chromatography (silica gel, 80 g, 0% to 20% AcOEt in heptane) to afford the title compound (4.37 g, 76.05%) as white solid. MS (ESI): m/z=279.9 [M-C4H8+H]⁺.

Step 2: tert-Butyl 6-[[1-methyl-5-(trifluoromethyl)pyrazol-4-yl]methylene]-2-azaspiro[3.3]heptane-2-carboxylate

4-Bromo-1-methyl-5-(trifluoromethyl)pyrazole (CAS: 497832-98-1) (2.05 g, 8.95 mmol), tert-butyl 6-[(4,4,5,5-tetramethyl-1,3,2-dioxaborolan-2-yl)methylene]-2-azaspiro[3.3]heptane-2-carboxylate (2.5 g, 7.46 mmol), 1,1′-bis(diphenylphosphino) ferrocene-palladium(II) dichloride dichloromethane complex (1.22 g, 1.49 mmol) and potassium carbonate (2.06 g, 14.91 mmol) were dissolved in 1,4-Dioxane (43 mL) and water (8 mL). The reaction mixture was heated to 90° C. under argon for 18 hours. The reaction mixture was concentrated under reduced pressure. The residue was partitioned between EtOAc and water. The organic layer was washed with brine. The extract was dried over sodium sulfate, filtered through a thin layer of silica gel and evaporated. The crude product was purified by FC (120 g SiO2, hexane/MtBE with MtBE from 0~70%, flow rate=85 mL/min) to afford the title compound (1.4 g, 3.92 mmol, 49.91% yield) as white solid. MS (ESI): m/z=358.0 [M+H]⁺.

Step 3: tert-Butyl 6-[[1-methyl-5-(trifluoromethyl)pyrazol-4-yl]methyl]-2-azaspiro[3.3]heptane-2-carboxylate

To the solution of tert-butyl 6-[[1-methyl-5-(trifluoromethyl)pyrazol-4-yl]methylene]-2-azaspiro[3.3]heptane-2-carboxylate (5200.0 mg, 14.55 mmol) in EtOAc (80 mL) was added Pd/C (1500.0 mg, 1.41 mmol) under nitrogen atmosphere. The mixture was purged with hydrogen for 3 times and then stirred at 20° C. for 0.5 h under hydrogen atmosphere with a ballon. The palladium catalyst was filtered off and the filtrate was dried in vacuo to give the title compound (5100.0 mg, 14.19 mmol, 97.53% yield) as a yellow oil. MS (ESI): m/z=304.1, [M-C4H8+H]⁺.

Step 4:4-Methylbenzenesulfonic acid; (6S)-6-[[3-(trifluoromethylsulfonyl)phenyl]methyl]-2-azaspiro[3.4]octane

To a solution of tert-butyl 6-[[1-methyl-5-(trifluoromethyl)pyrazol-4-yl]methyl]-2-azaspiro[3.3]heptane-2-carboxylate (1.69 g, 4.7 mmol) in EtOAc (17 mL) was added 4-methylbenzenesulfonic acid (890.73 mg, 5.17 mmol) which was then stirred at 80° C. for 12 hours. The reaction mixture was concentrated under reduced pressure to give a crude residue. The residue was mixed with 20 mL deionized water and 2 mL CAN followed by lyophilization to give the title compound (1974.0 mg, 4.58 mmol, 92.04% yield) as a light yellow solid. MS (ESI): m/z=260.0 [M+H]⁺.

### BB112

### 3-[3-(4-Fluorophenyl)-1-bicyclo[1.1.1]pentanyl]azetidine; 2,2,2-trifluoroacetic Acid

To a solution of 3-[3-(4-fluorophenyl)-1-bicyclo[1.1.1]pentanyl]azetidine-1-carboxylic acid tert-butyl ester (371 mg, 0.994 mmol, 1.000 eq) in dichloromethane (5 mL) was added TFA (1.13 g, 765.43 μL, 9.94 mmol, 10.000 eq) and the reaction mixture was stirred at room temperature for 18 hours. Volatiles were removed in vacuo to give 587 mg of the crude title compound as colorless viscous oil (purity ~55% major contaminant excess of TFA), which was used without further purification. MS (ESI): m/z=218.1 [M+H]⁺.

Step 1: tert-Butyl 3-[3-(4-fluorophenyl)-1-bicyclo[1.1.1]pentanyl]azetidine-1-carboxylate

To a flask was added 3-(3-iodo-1-bicyclo[1.1.1]pentanyl)azetidine-1-carboxylic acid tert-butyl ester (500 mg, 1.43 mmol) and Fe(acac)₃ (101.13 mg, 0.286 mmol) after which the atmosphere was switched to an argon atmosphere followed by addition of THF, extra dry (1.5 mL) and TMEDA (85.9 μL, 0.573 mmol). To the stirred reaction solution was then slowly added 0.500 M solution of 4-fluorophenylmagnesium bromide in THFe (4.58 mL, 2.29 mmol) with a syringe pump over 45 minutes after which the reaction was stirred at room temperature for 2 hours. The reaction was quenched by addition of a few drops of saturated aqueous NH₄Cl solution and stirred at room temperature for few minutes. The crude reaction solution was poured into a separating funnel containing EtOAc and saturated aqueous NH₄Cl solution for extraction. The organic phase was collected and the aqueous phase was back-extracted with EtOAc. The combined organic phases were dried over sodium sulfate and evaporated down to dryness to give 754 mg of the crude desired product as a dark orange oil. The crude material was purified by flash chromatography with a 80 g SiO2 column, eluent mixture of heptane and TBME (0% to 20%) to give 371 mg of the title product as a white solid. MS (ESI): m/z=262.2 [M-C4H8+H]⁺.

Step 2:3-[3-(4-Fluorophenyl)-1-bicyclo[1.1.1]pentanyl]azetidine; 2,2,2-trifluoroacetic Acid

To a solution of tert-butyl 3-[3-(4-fluorophenyl)-1-bicyclo[1.1.1]pentanyl]azetidine-1-carboxylate (371 mg, 0.994 mmol) in dichloromethane (5 mL) was added TFA (1.13 g, 765.43 uL, 9.94 mmol) and the reaction mixture was stirred at room temperature for 18 hours. Volatiles were removed in vacuo to give 587 mg of the crude title compound as colorless viscous oil (purity ~55% major contaminant excess of TFA), which was used without further purification. MS (ESI): m/z=218.1 [M+H]⁺.

### BB113

### 2,2,2-Trifluoroacetic acid; 6-[[4-(trifluoromethylsulfonyl)phenyl]methyl]-2-azaspiro[3.3]heptane

Step 1: tert-Butyl 6-[(4,4,5,5-tetramethyl-1,3,2-dioxaborolan-2-yl)methylene]-2-azaspiro[3.3]heptane-2-carboxylate

To a solution of 2,2,6,6-tetramethylpiperidine (3.3 mL, 19.54 mmol) in THF (30 mL) was added 1.6 M BuLi (12.21 mL, 19.54 mmol) between −45° C. and −35° C. The mixture was stirred and allowed to warm up to 5° C. The reaction mixture was cooled to −74° C. A solution of 4,4,5,5-tetramethyl-2-[(4,4,5,5-tetramethyl-1,3,2-dioxaborolan-2-yl)methyl]-1,3,2-dioxaborolane (4.8 g, 17.91 mmol) in THF (15 mL) was added dropwise below −70° C. The reaction mixture was stirred at −74° C. for 30 minutes. A solution of 6-keto-2-azaspiro[3.3]heptane-2-carboxylic acid tert-butyl ester (3.44 g, 16.28 mmol) in THF (22 mL) was added dropwise below −70° C. in 45 minutes. The reaction mixture was allowed to warm up to room temperature and stirred at room temperature for 3 hours. The reaction mixture was poured on saturated aqueous NH₄Cl solution (210 ml). Aqueous HCl 2 M solution was added to adjust the pH to 6. The mixture was extracted with EtOAc. The organic layer was washed with H₂O, brine, dried over Na2SO4 and concentrated in vacuo to give 7.2 g of the crude title compound as a yellow solid. The crude material was purified by flash chromatography (silica gel, 80 g, 0% to 20% AcOEt in heptane) to afford the title compound (4.37 g, 76.05%) as white solid. MS (ESI): m/z=279.9 [M-C4H8+H]⁺.

Step 2: tert-Butyl 6-[[4-(trifluoromethylsulfonyl)phenyl]methylene]-2-azaspiro[3.3]heptane-2-carboxylate

To a solution of 1-bromo-4-(trifluoromethylsulfonyl)benzene (CAS: 312-20-9) (500.0 mg, 1.73 mmol) in 1,4-dioxane (5 mL) and water (1 mL) was added tert-butyl 6-[(4,4,5,5-tetramethyl-1,3,2-dioxaborolan-2-yl)methylene]-2-azaspiro[3.3]heptane-2-carboxylate (695.85 mg, 2.08 mmol), K₂CO₃ (597.65 mg, 4.32 mmol) and Pd(dppf)Cl₂·CH2Cl2 (141.25 mg, 0.17 mmol) under nitrogen atmosphere. The mixture was stirred at 80° C. for 2 hours under nitrogen atmosphere. The reaction was purified by column chromatography on silica gel (eluted with petroleum ether/EtOAc=0-5%) to give the title compound (674.0 mg, 1.61 mmol, 92.41% yield) as a yellow solid. MS (ESI): m/z=362.0 [M-C4H8+H]⁺.

Step 3: tert-Butyl 6-[[4-(trifluoromethylsulfonyl)phenyl]methyl]-2-azaspiro[3.3]heptane-2-carboxylate

To a solution of tert-butyl 6-[[4-(trifluoromethylsulfonyl)phenyl]methylene]-2-azaspiro[3.3]heptane-2-carboxylate (5900.0 mg, 14.13 mmol) in EtOAc (59 mL) was added Pd/C (wet) (1997.0 mg) at 25° C. Then the reaction was stirred at 25° C. under hydrogen atmosphere with a balloon for 1 hour. The reaction mixture was filtered, the filtrate was concentrated under reduced pressure to give the tile compound (5811.0 mg, 13.85 mmol, 98.02% yield) as a yellow solid. MS (ESI): m/z=364.0, [M-C4H8+H]⁺.

Step 4:2,2,2-Trifluoroacetic acid; 6-[[4-(trifluoromethylsulfonyl)phenyl]methyl]-2-azaspiro[3.3]heptane

To a solution of tert-butyl 6-[[4-(trifluoromethylsulfonyl)phenyl]methyl]-2-azaspiro[3.3]heptane-2-carboxylate (460 mg, 1.1 mmol) in dichloromethane (5 mL) was added TFA (844.86 μL, 10.97 mmol) and the reaction mixture was stirred at room temperature for 18 hours. Volatiles were removed in vacuo to give 938 mg of the crude title product (purity ~50%, major contaminant excess of TFA) as a yellow viscous oil, which was used without further purification. MS (ESI): m/z=320.1 [M+H]⁺.

### BB114

### 2,2,2-Trifluoroacetic acid; 3-[3-[3-(trifluoromethoxy)phenyl]-1-bicyclo[1.1.1]pentanyl]azetidine

Step 1: tert-Butyl 3-[3-[3-(trifluoromethoxy)phenyl]-1-bicyclo[1.1.1]pentanyl]azetidine-1-carboxylate

To a flask was added 3-(3-iodo-1-bicyclo[1.1.1]pentanyl)azetidine-1-carboxylic acid tert-butyl ester (CAS: 2375261-02-0) (500 mg, 1.43 mmol) and Fe(acac)₃ (101.13 mg, 0.286 mmol) after which the atmosphere was switched to an argon atmosphere followed by addition of anhydrous THF (1.5 mL) and TMEDA (85.9 μL, 0.573 mmol). To the stirred reaction solution was then slowly added 0.500 M solution in THF of bromo-[3-(trifluoromethoxy)phenyl]magnesium (4.58 mL, 2.29 mmol) with a syringe pump over 60 min after which the reaction was stirred at room temperature for 1 hour. The reaction was quenched by addition of a few drops of saturated aqueous NH4Cl solution and stirred at room temperature for few minutes. The crude reaction solution was poured into a separating funnel containing EtOAc and saturated aqueous NH4Cl solution for extraction. The organic phase was collected and the aqueous phase was back-extracted with EtOAc. The combined organic phases were dried over sodium sulfate and evaporated down to dryness to give 805 mg of the crude desired product as a dark orange oil. The crude material was purified by flash chromatography with a 80 g SiO2 column, eluent mixture of heptane and TBME (0% to 20%) to give 445 mg of the desired product as a light yellow viscous oil but not pure enough. The fraction was submitted for a purification by SFC to yield 338 mg of the title compound as a colorless gum. MS (ESI): m/z=328.1 [M-C4H8+H]⁺.

Step 1:2,2,2-Trifluoroacetic acid; 3-[3-[3-(trifluoromethoxy)phenyl]-1-bicyclo[1.1.1]pentanyl]azetidine

To a solution of 3-[3-[3-(trifluoromethoxy)phenyl]-1-bicyclo[1.1.1]pentanyl]azetidine-1-carboxylic acid tert-butyl ester (335 mg, 0.874 mmol) in dichloromethane (4 mL) was added TFA (673.15 μL, 8.74 mmol) and the reaction mixture was stirred at room temperature for 18 hours. Volatiles were removed in vacuo to give 470 mg of the crude title product (purity ~73%, major contaminant excess of TFA) which was used without further purification. MS (ESI): m/z=284.1 [M+H]⁺.

### BB115

### 2,2,2-Trifluoroacetic acid; 3-[3-[4-(trifluoromethylsulfonyl)phenyl]-1-bicyclo[1.1.1]pentanyl]azetidine

Step 1: tert-Butyl 3-[3-(1,3-dioxoisoindolin-2-yl)oxycarbonyl-1-bicyclo[1.1.1]pentanyl]azetidine-1-carboxylate

To a solution of 3-(1-tert-butoxycarbonylazetidine-3-yl) bicyclo[1.1.1]pentane-1-carboxylic acid (CAS: 2227205-20-9) (5 g, 18.7 mmol) in dichloromethane (92 mL) was added 2-hydroxyisoindoline-1,3-quinone (3.36 g, 20.57 mmol), dicyclohexylcarbodiimide (4.25 g, 20.57 mmol) and DMAP (228.51 mg, 1.87 mmol) after which the reaction mixture was stirred at room temperature for 18 hours. The reaction volume was reduced in vacuo, then absorbed onto H-MN Isolute. The crude material was then directly purified by flash chromatography with a 330 g SiO2 column, eluent dichloromethane and EtOAc (5% to 15%) to give 7.28 g of the title compound as a white solid. MS (ESI): m/z=357.1 [M-C4H8+H]⁺.

Step 2: tert-Butyl 3-[3-[4-(trifluoromethylsulfonyl)phenyl]-1-bicyclo[1.1.1]pentanyl]azetidine-1-carboxylate

To a microwave vial was added 3-(3-phthalimidooxycarbonyl-1-bicyclo[1.1.1]pentanyl)azetidine-1-carboxylic acid tert-butyl ester (800 mg, 1.84 mmol), 1-bromo-4-triflyl-benzene (1.07 g, 3.69 mmol), Ni(dtbbpy)Br2 (179.44 mg, 368.54 μmol), NaHCO3 (619.14 mg, 7.37 mmol) and 2,6-dimethyl-1,4-dihydropyridine-3,5-dicarboxylic acid diethyl ester (933.47 mg, 3.69 mmol) after which the reaction vial was sealed and the atmosphere was changed to an argon atmosphere by repeating vacuum evacuation/argon refill 3 times followed by addition of anhydrous N,N-dimethylacetamide (18.41 mL). The reaction mixture was then sparged with argon for 5 minutes and then stirred in a photoreactor under 395 nm LED irradiation (stirring 1000 rpm, fan speed 6800, LED lamp intensity 50%) for 16 hours. Volatiles were removed in vacuo, the crude residue was then partitioned between MTBE and aqueous NaOH 1 M solution. The organic phase was collected and the aqueous phase was back-extracted with MTBE. The combined organic phases were dried over sodium sulfate and evaporated down to dryness to give 1.75 g of the crude desired product. The crude material was purified by flash chromatography with a 80 g SiO2 column, eluent mixture of heptane and TBME (5% to 50%) to give 436 mg of the title compound as a light yellow viscous oil. MS (ESI): m/z=376.1 [M-C4H8+H]⁺.

Step 3:2,2,2-Trifluoroacetic acid; 3-[3-[4-(trifluoromethylsulfonyl)phenyl]-1-bicyclo[1.1.1]pentanyl]azetidine

To a solution of 3-[3-(4-triflylphenyl)-1-bicyclo[1.1.1]pentanyl]azetidine-1-carboxylic acid tert-butyl ester (435 mg, 957.77 μmol) in dichloromethane (4 mL) was added TFA (737.87 μL, 9.58 mmol) and the reaction mixture was stirred at RT for 18 hours. Volatiles were removed in vacuo to yield 593 mg of the crude title compound as a light yellow viscous oil which was used without further purification (purity ~70% major contaminant excess of TFA). MS (ESI): m/z=332.0 [M+H]⁺.

### BB116

### N-[3-(Azetidin-3-yl)-1-bicyclo[1.1.1]pentanyl]-5-(trifluoromethyl)pyrazin-2-amine; 2,2,2-trifluoroacetic Acid

Step 1: tert-Butyl 3-[3-(benzyloxycarbonylamino)-1-bicyclo[1.1.1]pentanyl]azetidine-1-carboxylate

To a solution of 3-(1-tert-butoxycarbonylazetidine-3-yl) bicyclo[1.1.1]pentane-1-carboxylic acid (2.0 g, 7.48 mmol) and benzyl alcohol (1618.14 mg, 14.96 mmol) in toluene (50 mL) at ambient temperature was added triethylamine (3.13 mL, 22.45 mmol). The mixture was stirred for 5 minutes, and diphenylphosphonic azide (1.69 mL, 7.86 mmol) was added. The mixture was stirred another 15 minutes at ambient temperature, and for 16 hours at 100° C. After cooling, it was poured into ice/water (50 mL) and then extracted with MTBE. The organic layer was washed with H₂O and brine, dried over Na2SO4, filtered, and concentrated under vacuum. Crude product was purified by FC to provide the title compound (2.35 g, 6.31 mmol, 80.11% yield) as colorless viscous oil. MS (ESI): m/z=273.0 [M-BOC+H]⁺.

Step 2: tert-Butyl 3-(3-amino-1-bicyclo[1.1.1]pentanyl)azetidine-1-carboxylate

To a solution of tert-butyl 3-[3-(benzyloxycarbonylamino)-1-bicyclo[1.1.1]pentanyl]azetidine-1-carboxylate (4.15 g, 11.14 mmol) in methanol (50 mL) was added palladium on carbon (10%) (0.58 mL, 0.56 mmol). The reaction mixture was stirred 48 hours at room temperature under hydrogen atmosphere. The solids were removed by filtration and the filtrate was concentrated in vacuo, to give the title compound (2.6 g, 10.91 mmol, 93.01% yield) as a colorless oil. MS (ESI): m/z=239.2 [M+H]⁺.

Step 3: tert-Butyl 3-[3-[[5-(trifluoromethyl)pyrazin-2-yl]amino]-1-bicyclo[1.1.1]pentanyl]azetidine-1-carboxylate

To a solution of tert-butyl 3-(3-amino-1-bicyclo[1.1.1]pentanyl)azetidine-1-carboxylate (150 mg, 0.629 mmol) and DIPEA (219.64 μL, 1.26 mmol) in anhydrous DMF (3 mL) cooled down to 0° C. was added 2-fluoro-5-(trifluoromethyl)pyrazine (146.34 mg, 0.881 mmol) after which the reaction mixture was stirred at 0° C. for 10 minutes. The reaction was allowed to warm up to room temperature and stirred at room temperature for 3 hours. Volatiles were removed in vacuo to give a crude residue which was directly purified by flash chromatography with a 24 g SiO2 column, eluent mixture of heptane and EtOAc (10% to 60%) to give 196 mg of the title product as a white solid. MS (ESI): m/z=329.2 [M-C4H8+H]⁺.

Step 4: N-[3-(Azetidin-3-yl)-1-bicyclo[1.1.1]pentanyl]-5-(trifluoromethyl)pyrazin-2-amine; 2,2,2-trifluoroacetic Acid

To a solution of 3-[3-[[5-(trifluoromethyl)pyrazin-2-yl]amino]-1-bicyclo[1.1.1]pentanyl]azetidine-1-carboxylic acid tert-butyl ester (245 mg, 0.625 mmol) in dichloromethane (3 mL) was added tfa (481.2 μL, 6.25 mmol) and the reaction mixture was stirred at room temperature for 18 hours. Volatiles were removed in vacuo to give 493 mg of the crude title product as colorless viscous oil (purity ~50% major contaminant excess of TFA), which was used without further purification. MS (ESI): m/z=285.1 [M+H]⁺.

### BB117

### 4-Methylbenzenesulfonic acid; 6-[[5-(trifluoromethylsulfonyl)-2-pyridyl]methyl]-2-azaspiro[3.3]heptane

Step 1: tert-Butyl 6-[(4,4,5,5-tetramethyl-1,3,2-dioxaborolan-2-yl)methylene]-2-azaspiro[3.3]heptane-2-carboxylate

To a solution of 2,2,6,6-tetramethylpiperidine (2.76 g, 3.3 mL, 19.54 mmol) in THF (30 mL) was added 1.6 M BuLi (12.21 mL, 19.54 mmol) between −45° C. and −35° C. The mixture was stirred and allowed to warm up to 5° C. The reaction mixture was cooled to −74° C. A solution of 4,4,5,5-tetramethyl-2-[(4,4,5,5-tetramethyl-1,3,2-dioxaborolan-2-yl)methyl]-1,3,2-dioxaborolane (4.8 g, 17.91 mmol) in THF (15 mL) was added dropwise below −70° C. The reaction mixture was stirred at −74° C. for 30 minutes. A solution of 6-keto-2-azaspiro[3.3]heptane-2-carboxylic acid tert-butyl ester (3.44 g, 16.28 mmol) in THF (22 mL) was added dropwise below −70° C. in 45 minutes. The reaction mixture was allowed to warm up to room temperature and stirred at room temperature for 3 hours. The reaction mixture was poured on saturated aqueous NH₄Cl solution (210 ml). Aqueous HCl 2 M solution was added to adjust the pH to 6. The mixture was extracted with EtOAc. The organic layer was washed with H2O, brine, dried over Na2SO4 and concentrated in vacuo to give 7.2 g of the crude title compound as a yellow solid. The crude material was purified by flash chromatography (silica gel, 80 g, 0% to 20% AcOEt in heptane) to afford the title compound (4.37 g, 76.05%) as white solid. MS (ESI): m/z=279.9 [M-C4H8+H]⁺.

### Step 2:2-Chloro-5-(trifluoromethylsulfanyl)pyridine

To a solution of 2-chloro-5-iodopyridine (5.0 g, 20.88 mmol) in acetonitrile (20 mL) was add bpy (652.27 mg, 4.18 mmol), trifluoromethylthiosilver(I) (1047.14 mg, 5.01 mmol) and CuI (795.4 mg, 4.18 mmol). The mixture was stirred at 90° C. for 12 hours under nitrogen atmosphere. The reaction mixture was filtered and the filtrate was evaporated down to dryness to give the crude title compound (4.46 g, 20.88 mmol, 100.0% yield). The crude was used in the next step of the reaction directly without further purification. MS (ESI): m/z=213.9 [M+H]⁺.

### Step 3:2-Chloro-5-(trifluoromethylsulfonyl)pyridine

To a solution of 2-chloro-5-(trifluoromethylsulfanyl)pyridine (446.0 mg, 2.09 mmol) in 1,2-dichloroethane (9 mL), acetonitrile (9 mL) and water (18 mL) was cooled down to 0° C. followed by addition of sodium periodate (1339.75 mg, 6.26 mmol) and ruthenium (III) chloride hydrate (4.71 mg, 0.02 mmol) after which the mixture was stirred at 20° C. for 12 hours. The reaction mixture was added to 20 mL water and then extracted with EtOAc (40 mL×3). The combined organic phases were concentrated under vacuum to give a crude residue. The crude material was purified by chromatography on silica gel (PE:EtOAc=5:1, UV) to give the title compound (1.6 g, 6.51 mmol, 312.0% yield) as off-white solid. MS (ESI): m/z=245.9 [M+H]⁺.

Step 4: tert-Butyl 6-[[5-(trifluoromethylsulfonyl)-2-pyridyl]methylene]-2-azaspiro[3.3]heptane-2-carboxylate

To a solution of tert-butyl 6-[(4,4,5,5-tetramethyl-1,3,2-dioxaborolan-2-yl)methylene]-2-azaspiro[3.3]heptane-2-carboxylate (47.77 mg, 0.14 mmol) and 2-chloro-5-(trifluoromethylsulfonyl)pyridine (35.0 mg, 0.14 mmol), potassium carbonate (39.39 mg, 0.29 mmol) in 1,4-Dioxane (0.5 mL) and water (0.1 mL) was added 1,1′-bis(diphenylphosphino) ferrocene-palladium(II) dichloride dichloromethane complex (11.63 mg, 0.01 mmol), the mixture was stirred at 80° C. under nitrogen atmosphere for 12 hours. The reaction mixture was concentrated down to dryness and the crude residue was purified by t-HPLC (PE:EtOAc=3:1, UV) to give the title compound (50.0 mg, 0.12 mmol, 99.5% yield) as yellow solid. MS (ESI): m/z=363.0 [M-C4H8+H]⁺.

Step 5: tert-Butyl 6-[[5-(trifluoromethylsulfonyl)-2-pyridyl]methyl]-2-azaspiro[3.3]heptane-2-carboxylate

To a solution of tert-butyl 6-[[5-(trifluoromethylsulfonyl)-2-pyridyl]methylene]-2-azaspiro[3.3]heptane-2-carboxylate (7.5 g, 17.92 mmol) in EtOAc (65 mL) was added Pd/C (2.54 g, 2.39 mmol) at 20° C., under hydrogen atmosphere (15 Psi) for 4 hours. The reaction mixture was filtered and the filtrate was concentrated under reduced pressure to give crude product. The crude residue was purified by silica gel chromatography (Petroleum ether/EtOAc=70%) and the fractions were concentrated in vacuo to give the title compound (7.5 g, 17.84 mmol, 99.52% yield) as a brown solid. MS (ESI): m/z=365.1 [M-C4H8+H]⁺.

Step 6:4-Methylbenzenesulfonic acid; 6-[[5-(trifluoromethylsulfonyl)-2-pyridyl]methyl]-2-azaspiro[3.3]heptane

To a solution of tert-butyl 6-[[5-(trifluoromethylsulfonyl)-2-pyridyl]methyl]-2-azaspiro[3.3]heptane-2-carboxylate (7.5 g, 17.84 mmol) in EtOAc (75 mL) was added p-toluenesulfonic acid (7.06 g, 41.03 mmol, 2.3 eq) at 20° C. The mixture was stirred at 80° C. for 4 hours. The mixture was concentrated to remove the solvent, then deionized water was added to the obtained residue followed by lyophilized to give the title compound (10950.0 mg, 16.47 mmol, 86.81% yield) as brown gum. MS (ESI): m/z=321.1 [M+H]⁺.

### BB118

### 2,2,2-Trifluoroacetic acid; 6-[5-[[3-(trifluoromethyl)azetidin-1-yl]pyrazin-2-yl]methyl]-2-azaspiro[3.3]heptane

Step 1: tert-Butyl 6-[(4,4,5,5-tetramethyl-1,3,2-dioxaborolan-2-yl)methylene]-2-azaspiro[3.3]heptane-2-carboxylate

To a solution of 2,2,6,6-tetramethylpiperidine (3.3 mL, 19.54 mmol) in THF (30 mL) was added 1.6 M BuLi (12.21 mL, 19.54 mmol) between −45° C. and −35° C. The mixture was stirred and allowed to warm up to 5° C. The reaction mixture was cooled to −74° C. A solution of 4,4,5,5-tetramethyl-2-[(4,4,5,5-tetramethyl-1,3,2-dioxaborolan-2-yl)methyl]-1,3,2-dioxaborolane (4.8 g, 17.91 mmol) in THF (15 mL) was added dropwise below −70° C. The reaction mixture was stirred at −74° C. for 30 minutes. A solution of 6-keto-2-azaspiro[3.3]heptane-2-carboxylic acid tert-butyl ester (3.44 g, 16.28 mmol) in THF (22 mL) was added dropwise below −70° C. in 45 minutes. The reaction mixture was allowed to warm up to room temperature and stirred at room temperature for 3 hours. The reaction mixture was poured on saturated aqueous NH₄Cl solution (210 ml). Aqueous HCl 2 M solution was added to adjust the pH to 6. The mixture was extracted with EtOAc. The organic layer was washed with H₂O, brine, dried over Na2SO4 and concentrated in vacuo to give 7.2 g of the crude title compound as a yellow solid. The crude material was purified by flash chromatography (silica gel, 80 g, 0% to 20% AcOEt in heptane) to afford the title compound (4.37 g, 76.05%) as white solid. MS (ESI): m/z=279.9 [M-C4H8+H]⁺.

### Step 2:2-Bromo-5-[3-(trifluoromethyl)azetidin-1-yl]pyrazine

To a solution of 3-(trifluoromethyl)azetidine; hydrochloride (1000.0 mg, 6.15 mmol, 1.0 eq) and DIPEA (3106.55 mg, 30.76 mmol) in DMF (20 mL) was added 2,5-dibromopyrazine (1902.34 mg, 8.0 mmol), then the resulting solution was stirred at 100° C. for 2 hours. The reaction mixture was poured into saturated NaCl solution, then EtOAc was added to the reaction mixture. Then the mixture was poured into separating funnel for extraction and the organic phase was collected. Aqueous phase was back-extracted with EtOAc (three times) and the combined organic phases were dried over Na2SO4, concentrated under vacuum to give the crude product, which was then purified by column chromatography (SiO₂, eluent Petroleum ether/EtOAc) to give the title compound (1700.0 mg, 6.03 mmol, 75.39% yield) as a yellow solid. MS (ESI): m/z=281.9 [M+H]⁺.

Step 3: tert-Butyl 6-[[5-[3-(trifluoromethyl)azetidin-1-yl]pyrazin-2-yl]methylene]-2-azaspiro[3.3]heptane-2-carboxylate

To a solution of 2-bromo-5-[3-(trifluoromethyl)azetidin-1-yl]pyrazine (500.0 mg, 1.77 mmol) and tert-butyl 6-[(4,4,5,5-tetramethyl-1,3,2-dioxaborolan-2-yl)methylene]-2-azaspiro[3.3]heptane-2-carboxylate (713.15 mg, 2.13 mmol), potassium carbonate (489.99 mg, 3.55 mmol) in 1,4-Dioxane (10 mL) and water (2 mL) was added 1,1′-bis(diphenylphosphino) ferrocene-palladium(II) dichloride dichloromethane complex (144.65 mg, 0.18 mmol), the mixture was degassed with nitrogen for few minutes and stirred at 100° C. under nitrogen atmosphere for 12 hours. The reaction was purified by flash chromatography on silica gel eluting with PE/EtOAc (1:1) (TLC, PE:EtOAc=1:1, Rf=0.3) to the title compound (510.0 mg, 1.24 mmol, 70.1% yield) as a light-yellow solid. MS (ESI): m/z=355.1 [M-C4H8+H]⁺.

Step 4: tert-Butyl 6-[[5-[3-(trifluoromethyl)azetidin-1-yl]pyrazin-2-yl]methyl]-2-azaspiro[3.3]heptane-2-carboxylate

To a solution of tert-butyl 6-[5-[[3-(trifluoromethyl)azetidin-1-yl]pyrazin-2-yl]methylene]-2-azaspiro[3.3]heptane-2-carboxylate (1450.0 mg, 3.53 mmol) in EtOAc (40 mL) was added wet Pd/C (500.0 mg, 3.53 mmol) under nitrogen atmosphere. The suspension was degassed under vacuum and purged with hydrogen, the procedure was repeated 3 times. The reaction was stirred under hydrogen atmosphere (15 psi) at 25° C. for 0.3 hour. The reaction mixture was filtered and the filtrate was concentrated under reduced pressure to give the title compound (1217.0 mg, 2.95 mmol, 80.76% yield) as a light yellow solid. MS (ESI): m/z=357.1 [M-C4H8+H]⁺.

Step 5:2,2,2-Trifluoroacetic acid; 6-[[5-[3-(trifluoromethyl)azetidin-1-yl]pyrazin-2-yl]methyl]-2-azaspiro[3.3]heptane

To a solution of 6-[[5-[3-(trifluoromethyl)azetidin-1-yl]pyrazin-2-yl]methyl]-2-azaspiro[3.3]heptane-2-carboxylic acid tert-butyl ester (75.37 mg, 0.183 mmol) in dry dichloromethane (0.536 mL) was added TFA (268.23 μL, 3.48 mmol). The reaction mixture was allowed to stir for 1 hour before concentration under reduced pressure followed by azeotropic removal of excess TFA with dichloromethane (3×) to give the title compound which was used in the subsequent reaction without further purification. MS (ESI): m/z=313.2 [M+H]⁺.

### BB119

### 5-(2-Azaspiro[3.3]heptan-6-ylmethyl)-N-[[1-(trifluoromethyl)cyclopropyl]methyl]pyrazin-2-amine; 2,2,2-trifluoroacetic Acid

Step 1: tert-Butyl 6-[(4,4,5,5-tetramethyl-1,3,2-dioxaborolan-2-yl)methylene]-2-azaspiro[3.3]heptane-2-carboxylate

To a solution of 2,2,6,6-tetramethylpiperidine (2.76 g, 3.3 mL, 19.54 mmol) in THF (30 mL) was added 1.6 M BuLi (12.21 mL, 19.54 mmol) between −45° C. and −35° C. The mixture was stirred and allowed to warm up to 5° C. The reaction mixture was cooled to −74° C. A solution of 4,4,5,5-tetramethyl-2-[(4,4,5,5-tetramethyl-1,3,2-dioxaborolan-2-yl)methyl]-1,3,2-dioxaborolane (4.8 g, 17.91 mmol) in THF (15 mL) was added dropwise below −70° C. The reaction mixture was stirred at −74° C. for 30 minutes. A solution of 6-keto-2-azaspiro[3.3]heptane-2-carboxylic acid tert-butyl ester (3.44 g, 16.28 mmol) in THF (22 mL) was added dropwise below −70° C. in 45 minutes. The reaction mixture was allowed to warm up to room temperature and stirred at room temperature for 3 hours. The reaction mixture was poured on saturated aqueous NH₄Cl solution (210 ml). Aqueous HCl 2 M solution was added to adjust the pH to 6. The mixture was extracted with EtOAc. The organic layer was washed with H2O, brine, dried over Na2SO4 and concentrated in vacuo to give 7.2 g of the crude title compound as a yellow solid. The crude material was purified by flash chromatography (silica gel, 80 g, 0% to 20% AcOEt in heptane) to afford the title compound (4.37 g, 76.05%) as white solid. MS (ESI): m/z=279.9 [M+H]⁺.

Step 2:5-Chloro-N-[[1-(trifluoromethyl)cyclopropyl]methyl]pyrazin-2-amine

To a solution of 2,5-dichloropyrazine (3400.0 mg, 22.82 mmol), [1-(trifluoromethyl)cyclopropyl]methanamine; hydrochloride (2003.53 mg, 11.41 mmol) in NMP (34 mL), was add DIPEA (19.88 mL, 114.11 mmol) at 20° C. and the reaction mixture was stirred at 130° C. for 12 hours. The reaction mixture was poured into water (60 mL) and then extracted with EtOAc (15 mL*3), the organic layer was washed with brine and dried over anhydrous sodium sulfate and concentrated under vacuum to give a crude residue. The residue was purified by flash chromatography on silica gel eluting with PE/EtOAc (3:1) (TLC, PE:EtOAc=3:1, Rf=0.60) to afford the title compound (1800.0 mg, 7.15 mmol, 31.34% yield) as a yellow oil, MS (ESI): m/z=252.1 [M+H]⁺.

Step 3: tert-Butyl 6-[[5-[[1-(trifluoromethyl)cyclopropyl]methylamino]pyrazin-2-yl]methylene]-2-azaspiro[3.3]heptane-2-carboxylate

To a solution of 5-chloro-N-[[1-(trifluoromethyl)cyclopropyl]methyl]pyrazin-2-amine (2145.0 mg, 8.52 mmol) and tert-butyl 6-[(4,4,5,5-tetramethyl-1,3,2-dioxaborolan-2-yl)methylene]-2-azaspiro[3.3]heptane-2-carboxylate (2571.93 mg, 7.67 mmol), K₂CO₃ (2356.06 mg, 17.05 mmol) in 1,4-dioxane (22 mL) and water (4.4 mL) was added Pd(dppf)Cl₂·CH2Cl2 (695.57 mg, 0.85 mmol), the mixture was degassed with nitrogen for few minutes and stirred at 100° C. under nitrogen atmosphere for 12 hours. Volatiles were removed in vacuo and the crude residue and was purified by SiO2 chromatography (petroleum ether:EtOAc=3:1 to 1:1) and concentrated under reduced pressure to give the crude product. The crude product was triturated with MeOH (10 mL) at 25° C. for 15 minutes and the precipitate was filtered off, dried in vacuo to give the title compound (1900.0 mg, 4.48 mmol, 52.51% yield) as a off-white solid. MS (ESI): m/z=425 [M+H]⁺.

Step 4: tert-Butyl 6-[[5-[[1-(trifluoromethyl)cyclopropyl]methylamino]pyrazin-2-yl]methyl]-2-azaspiro[3.3]heptane-2-carboxylate

To a solution of tert-butyl 6-[[5-[[1-(trifluoromethyl)cyclopropyl]methylamino]pyrazin-2-yl]methylene]-2-azaspiro[3.3]heptane-2-carboxylate (1800.0 mg, 4.24 mmol) in EtOAc (20 mL) was added Pd/C (wet) (600.0 mg, 0.56 mmol). under a nitrogen atmosphere. The reaction mixture was then stirred at 25° C. for 0.5 hour under hydrogen atmosphere with a balloon. The reaction mixture was filtered and the filtrate was concentrated under reduced pressure to afford the title compound (1795.0 mg, 4.21 mmol, 99.25% yield) as a yellow solid. MS (ESI): m/z=371.3 [M-C4H8+H]⁺.

Step 5:5-(2-Azaspiro[3.3]heptan-6-ylmethyl)-N-[[1-(trifluoromethyl)cyclopropyl methyl]pyrazin-2-amine; 2,2,2-trifluoroacetic Acid

To a solution of 6-[5-[[1-(trifluoromethyl)cyclopropyl]methylamino]pyrazin-2-yl]methyl]-2-azaspiro[3.3]heptane-2-carboxylic acid tert-butyl ester (18.59 mg, 0.044 mmol) in dry dichloromethane (0.500 mL) was added TFA (250 μL, 3.25 mmol). The reaction mixture was allowed to stir for 1 hour before concentration under reduced pressure followed by azeotropic removal of excess TFA with dichloromethane (3×) to give the crude title compound which was used in the subsequent reaction without further purification. MS (ESI): m/z=327.1 [M+H]⁺.

### Example 866

A compound of formula (I) can be used in a manner known per se as the active ingredient for the production of tablets of the following composition:

| | Per tablet | |
|---|---|---|
| Active ingredient | 200 | mg |
| Microcrystalline cellulose | 155 | mg |
| Corn starch | 25 | mg |
| Talc | 25 | mg |
| Hydroxypropylmethylcellulose | 20 | mg |
| | 425 | mg |

### Example 867

A compound of formula (I) can be used in a manner known per se as the active ingredient for the production of capsules of the following composition:

| | Per capsule | |
|---|---|---|
| Active ingredient | 100.0 | mg |
| Corn starch | 20.0 | mg |
| Lactose | 95.0 | mg |
| Talc | 4.5 | mg |
| Magnesium stearate | 0.5 | mg |
| | 220.0 | mg |

1. A compound of formula (I) 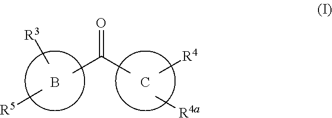
or a pharmaceutically acceptable salt thereof, wherein:
R⁵ is selected from a group 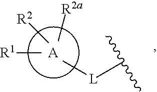
C₁₋₆-alkyl, C₁₋₆-alkoxy, halo-C₁₋₆-alkyl, halo-C₁₋₆-alkoxy, C₁₋₆-alkyl-SO₂—, C₁₋₆-alkyl-SO₂NH—;
R¹ is selected from hydrogen, halogen, SF₅, a group 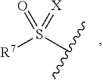
sulfamoyl, (C₁₋₆-alkyl)₂PO—C₁₋₆-alkyl-, a group 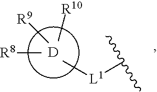
cyano, C₁₋₆-alkyl, C₁₋₆-alkoxy, halo-C₁₋₆-alkyl, halo-C₁₋₆-alkoxy, carbamoyl, oxo, C₁₋₆-alkyl-NHC(O)—, C₁₋₆-alkoxycarbonyl, and C₁₋₆-alkoxycarbonyl-C₁₋₆-alkyl-; C₃₋₁₀-cycloalkyl; and
R² is selected from hydrogen, halogen, cyano, C₁₋₆-alkyl, C₁₋₆-alkoxy, halo-C₁₋₆-alkyl, halo-C₁₋₆-alkoxy, and C₁₋₆-alkylsulfonyl; or
R¹ and R², taken together with the atom(s) to which they are attached, form a 3- to 14-membered heterocycle that is optionally substituted with one or more C₁₋₆-alkyl substituents;
R^{2a} is selected from hydrogen and halogen;
R³ is selected from hydrogen and C₁₋₆-alkyl;
R⁴ is selected from hydrogen, hydroxy, amino, 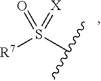
a group 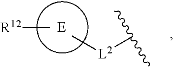
sulfamoyl, carboxy, hydroxy-C₁₋₆-alkyl, C₁₋₆-alkyl-SO₂NH—, carbamoyl, C₁₋₆-alkyl-NHC(O)—, (C₁₋₆-alkyl)₂NC(O)—, C₁₋₆-alkyl-C(O)NH—, C₁₋₆-alkoxy-C(O)NH—, and C₁₋₆-alkoxy-C(O)—;
R^{4a} is selected from hydrogen and C₁₋₆-alkyl;
R⁶ is selected from hydrogen, C₆₋₁₄-aryl and halo-C₆₋₁₄-aryl;
R⁷ is selected from C₁₋₆-alkyl, cyano-C₁₋₆-alkyl, and halo-C₁₋₆-alkyl;
R⁸ is selected from hydrogen, halogen, cyano, hydroxy, oxo, carboxy, C₁₋₆-alkoxycarbonyl, C₁₋₆-alkyl, a group 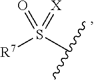
a group 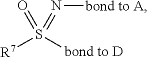
a group 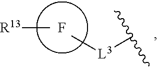
C₁₋₆-alkyl-SO—, C₁₋₆-alkyl-S—, C₁₋₆-alkyl-SO₂—C₁₋₆-alkyl-, (C₁₋₆-alkyl)₂-PO—, C₁₋₆-alkoxy, halo-C₁₋₆-alkyl, halo-C₁₋₆-alkoxy, carbamoyl, and C₁₋₆-alkyl-NHC(O)—;
R⁹ is selected from hydrogen, halogen, hydroxy, C₁₋₆-alkyl, and halo-C₁₋₆-alkyl;
R¹⁰ is selected from hydrogen and halogen;
R¹¹ is selected from hydrogen, methyl, cyclopropylmethyl, and phenyl;
R¹² is selected from hydrogen, halogen, oxo, C₁₋₆-alkyl, C₁₋₆-alkoxycarbonyl, and C₃₋₁₀-cycloalkyl;
R¹³ is selected from hydrogen, C₁₋₆-alkyl and halo-C₁₋₆-alkyl;
A is selected from C₆₋₁₄-aryl, C₃₋₁₀-cycloalkyl, 5- to 6-membered heteroaryl, and 3- to 14-membered heterocyclyl;
B is selected from azetidinyl, piperidyl, piperazinyl, 2-azaspiro[3.3]heptanyl, 2,6-diazaspiro[3.3]heptanyl, 2-azaspiro[3.4]octanyl, 6-azaspiro[3.4]octanyl, 2,7-diazaspiro[3.4]octanyl, 2,7-diazaspiro[3.5]nonanyl, 2-azaspiro[3.5]nonanyl, 7-azaspiro[3.5]nonanyl, 1,2,3,3a,4,5,6,6a-octahydrocyclopenta[c]pyrrolyl, cyclobutyl, cubanyl, spiro[3.3]heptanyl, a group 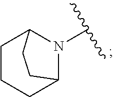 [SMILES: *N1C2CCCC1CC2]
and a group 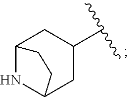 [SMILES: *C1CC2CCC(C1)N2]
C is selected from phenyl, azetidinyl, pyrrolidinyl, piperidyl, piperazinyl, morpholinyl, 4,5-dihydroisoxazolyl, 2-azabicyclo[2.1.1]hexanyl, 2-azaspiro[3.3]heptanyl, and 2,8-diazaspiro[3.5]nonanyl;
D is selected from C₆₋₁₄-aryl, C₃₋₁₀-cycloalkyl, 5-6-membered heteroaryl, and 3- to 14-membered heterocyclyl;
E is selected from 5- to 14-membered heteroaryl, 3- to 14-membered heterocyclyl, and C₃₋₁₀-cycloalkyl;
F is selected from C₃₋₁₀-cycloalkyl, 3- to 14-membered heterocyclyl, and 5- to 14-membered heteroaryl;
L is selected from a covalent bond, carbonyl, —CHR⁶—, —O—, —NR¹¹—, —NHCH₂—, —CH₂NR¹¹—, —N(CH₃)CH₂—, —OCH₂—, —CH₂O—, —CH₂OCH₂—, —CH₂CH₂—, —CF₂CH₂—, —CH₂CF₂—, —CH═CH—, —C≡C—, —SO₂—, —SO₂NH—, —SO₂NHCH₂—, —C(O)NH—, —NHC(O)—, —CH₂NHC(O)—, and 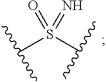 [SMILES: *S(*)(=N)=O]
L¹ is selected from a covalent bond, carbonyl, —CH₂—, —CH₂O—, —OCH₂—, —NHCH₂—, CH₂NH—, —NR¹¹—, —C(O)—NH—NH—C(O)—CH₂—, —SO₂—, and —O—;
L² is selected from a covalent bond and —SO₂—;
L³ is selected from a covalent bond, carbonyl, and —CH₂—;
X is selected from O and NH;
provided that when B is 2-azaspiro[3.3]heptanyl or 2,6-diazaspiro[3.3]heptanyl, L is not a covalent bond.

2. The compound of formula (I) according to claim 1, or a pharmaceutically acceptable salt thereof, wherein said compound of formula (I) is a compound of formula (I-I) 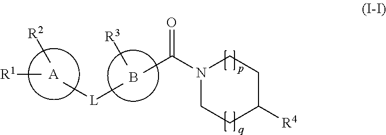
wherein R¹ to R⁴, A, B, and L are as defined in claim 1; and
p and q are each independently selected from 0 and 1.

3. The compound of formula (I) according to claim 1, or a pharmaceutically acceptable salt thereof, wherein:
R⁵ is selected from a group 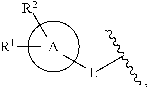
C₁₋₆-alkyl-SO₂—, C₁₋₆-alkyl-SO₂NH—, and halo-C₁₋₆-alkoxy;
R¹ is selected from hydrogen, halogen, SF₅, a group 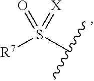
sulfamoyl, (C₁₋₆-alkyl)₂PO—C₁₋₆-alkyl-, a group 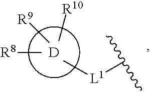
C₁₋₆-alkyl, C₁₋₆-alkoxy, halo-C₁₋₆-alkyl, halo-C₁₋₆-alkoxy, carbamoyl, oxo, C₁₋₆-alkyl-NHC(O)—, C₁₋₆-alkoxycarbonyl, and C₁₋₆-alkoxycarbonyl-C₁₋₆-alkyl-; and
R² is selected from hydrogen, halogen, cyano, C₁₋₆-alkyl, C₁₋₆-alkoxy, halo-C₁₋₆-alkyl, halo-C₁₋₆-alkoxy, and C₁₋₆-alkylsulfonyl; or
R¹ and R², taken together with the atom(s) to which they are attached, form a 3- to 14-membered heterocycle that is optionally substituted with 1-2 C₁₋₆-alkyl substituents;
R^{2a} is selected from hydrogen and halogen;
R⁶ is selected from hydrogen, C₆₋₁₄-aryl, and halo-C₆₋₁₄-aryl;
R⁷ is selected from C₁₋₆-alkyl, cyano-C₁₋₆-alkyl, and halo-C₁₋₆-alkyl;
R⁸ is selected from hydrogen, halogen, cyano, hydroxy, oxo, carboxy, C₁₋₆-alkoxycarbonyl, C₁₋₆-alkyl, a group 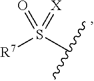
a group 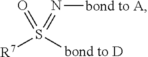
a group 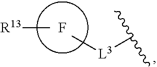
C₁₋₆-alkyl-SO—, C₁₋₆-alkyl-S—, C₁₋₆-alkyl-SO₂—C₁₋₆-alkyl-, (C₁₋₆-alkyl)₂-PO—, C₁₋₆-alkoxy, halo-C₁₋₆-alkyl, halo-C₁₋₆-alkoxy, carbamoyl, and C₁₋₆-alkyl-NHC(O)—;
R⁹ is selected from hydrogen, halogen, hydroxy, C₁₋₆-alkyl, and halo-C₁₋₆-alkyl;
R¹⁰ is selected from hydrogen and halogen;
R¹¹ is selected from hydrogen, methyl, cyclopropylmethyl, and phenyl;
R¹³ is selected from hydrogen, C₁₋₆-alkyl and halo-C₁₋₆-alkyl;
A is selected from C₆₋₁₄-aryl, C₃₋₁₀-cycloalkyl, 3- to 14-membered heterocyclyl, and 5- to 6-membered heteroaryl;
D is selected from C₆₋₁₄-aryl, C₃₋₁₀-cycloalkyl, 5-6-membered heteroaryl, and 3- to 14-membered heterocyclyl;
F is selected from C₃₋₁₀-cycloalkyl, 3- to 14-membered heterocyclyl, and 5- to 14-membered heteroaryl;
L is selected from a covalent bond, —CHR⁶—, —O—, —NR¹¹—, —NHCH₂—, —CH₂NR¹¹—, —OCH₂—, —CH₂O—, —CH₂OCH₂—, —CH₂CH₂—, —CF₂CH₂—, —CH₂CF₂—, —CH═CH—, —C≡C—, —SO₂—, —SO₂NH—, —SO₂NHCH₂—, —C(O)NH—, —NHC(O)—, —CH₂NHC(O)—, and 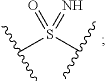 [SMILES: *S(*)(=N)=O]
L¹ is selected from a covalent bond, carbonyl, —CH₂—, —CH₂O—, —NHCH₂—, CH₂NH—, —NR¹¹—, —C(O)—NH—NH—C(O)—CH₂—, —SO₂—, and —O—;
L³ is selected from a covalent bond, carbonyl, and —CH₂—; and
X is selected from O and NH.

4. The compound of formula (I) according to claim 3, or a pharmaceutically acceptable salt thereof, wherein:
R⁵ is a group 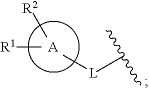
R¹ is selected from halogen, C₁₋₆-alkyl, halo-C₁₋₆-alkyl, and a group 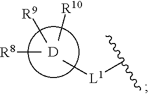
R² is selected from hydrogen, halogen, and halo-C₁₋₆-alkyl;
R^{2a} is hydrogen;
R⁸ and R⁹ are each independently selected from halogen and halo-C₁₋₆-alkyl;
R¹⁰ is hydrogen;
A is selected from C₆₋₁₄-aryl and C₃₋₁₀-cycloalkyl;
D is selected from C₆₋₁₄-aryl and C₃₋₁₀-cycloalkyl;
L is selected from a covalent bond, —O— and —CH₂O—; and
L¹ is a covalent bond.

5. The compound of formula (I) according to claim 4, or a pharmaceutically acceptable salt thereof, wherein:
R⁵ is a group 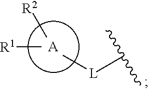
R¹ is selected from fluoro, chloro, tert-butyl, CHF₂, CF₃, and a group 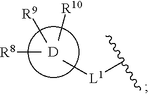
R² is selected from hydrogen, fluoro, chloro, and CF₃;
R^{2a} is hydrogen;
R⁸ and R⁹ are each independently selected from fluoro, chloro and CF₃;
R¹⁰ is hydrogen;
A is selected from phenyl and cyclopropyl;
D is selected from phenyl and cyclopropyl;
L is selected from a covalent bond, —O— and —CH₂O—; and
L¹ is a covalent bond.

6. The compound of formula (I) according to any one of claims 1 to 5, or a pharmaceutically acceptable salt thereof, wherein:
B is selected from spiro[3.3]heptan-2-yl, azetidinyl, and 7-azaspiro[3.5]nonan-7-yl; and
R³ is hydrogen.

7. The compound of formula (I) according to any one of claims 1 to 6, or a pharmaceutically acceptable salt thereof, wherein:
R⁴ is selected from a group 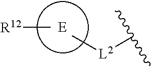
and carbamoyl;
R^{4a} is hydrogen;
R¹² is selected from hydrogen and oxo;
C is pyrrolidinyl;
E is selected from 5-membered heteroaryl and 5-membered heterocyclyl; and
L² is a covalent bond.

8. The compound of formula (I) according to claim 7, or a pharmaceutically acceptable salt thereof, wherein:
R⁴ is selected from a group 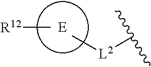
and carbamoyl;
R^{4a} is hydrogen;
R¹² is selected from hydrogen and oxo;
C is pyrrolidinyl;
E is selected from imidazolyl, pyrazolyl, triazolyl, and oxazolidinyl; and
L² is a covalent bond.

9. The compound of formula (I) according to claim 1, or a pharmaceutically acceptable salt thereof, wherein:
R⁵ is selected from a group 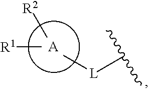
C₁₋₆-alkyl-SO₂—, C₁₋₆-alkyl-SO₂NH—, and halo-C₁₋₆-alkoxy;
R¹ is selected from hydrogen, halogen, SF₅, a group 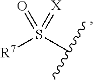
sulfamoyl, (C₁₋₆-alkyl)₂PO—C₁₋₆-alkyl-, a group 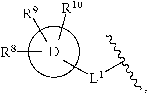
C₁₋₆-alkyl, C₁₋₆-alkoxy, halo-C₁₋₆-alkyl, halo-C₁₋₆-alkoxy, carbamoyl, oxo, C₁₋₆-alkyl-NHC(O)—, C₁₋₆-alkoxycarbonyl, and C₁₋₆-alkoxycarbonyl-C₁₋₆-alkyl-; and
R² is selected from hydrogen, halogen, cyano, C₁₋₆-alkyl, C₁₋₆-alkoxy, halo-C₁₋₆-alkyl, halo-C₁₋₆-alkoxy, and C₁₋₆-alkylsulfonyl; or
R¹ and R², taken together with the atom(s) to which they are attached, form a 3- to 14-membered heterocycle that is optionally substituted with 1-2 C₁₋₆-alkyl substituents;
R^{2a} is selected from hydrogen and halogen;
R³ is selected from hydrogen and C₁₋₆-alkyl;
R⁴ is selected from hydrogen, hydroxy, amino, a group 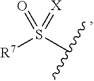
a group 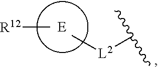
sulfamoyl, carboxy, hydroxy-C₁₋₆-alkyl, C₁₋₆-alkyl-SO₂NH—, carbamoyl, C₁₋₆-alkyl-NHC(O)—, (C₁₋₆-alkyl)₂NC(O)—, C₁₋₆-alkyl-C(O)NH—, C₁₋₆-alkoxy-C(O)NH—, and C₁₋₆-alkoxy-C(O)—;
R^{4a} is selected from hydrogen and C₁₋₆-alkyl;
R⁶ is selected from hydrogen, C₆₋₁₄-aryl, and halo-C₆₋₁₄-aryl;
R⁷ is selected from C₁₋₆-alkyl, cyano-C₁₋₆-alkyl, and halo-C₁₋₆-alkyl;
R⁸ is selected from hydrogen, halogen, cyano, hydroxy, oxo, carboxy, C₁₋₆-alkoxycarbonyl, C₁₋₆-alkyl, a group 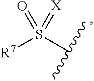
a group 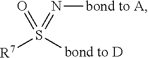
a group 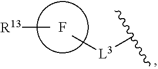
C₁₋₆-alkyl-SO—, C₁₋₆-alkyl-S—, C₁₋₆-alkyl-SO₂—C₁₋₆-alkyl-, (C₁₋₆-alkyl)₂-PO—, C₁₋₆-alkoxy, halo-C₁₋₆-alkyl, halo-C₁₋₆-alkoxy, carbamoyl, and C₁₋₆-alkyl-NHC(O)—;
R⁹ is selected from hydrogen, halogen, hydroxy, C₁₋₆-alkyl, and halo-C₁₋₆-alkyl;
R¹⁰ is selected from hydrogen and halogen;
R¹¹ is selected from hydrogen, methyl, cyclopropylmethyl, and phenyl;
R¹² is selected from hydrogen, halogen, oxo, C₁₋₆-alkyl, C₁₋₆-alkoxycarbonyl, and C₃₋₁₀-cycloalkyl;
R¹³ is selected from hydrogen, C₁₋₆-alkyl and halo-C₁₋₆-alkyl;
A is selected from C₆₋₁₄-aryl, C₃₋₁₀-cycloalkyl, 3- to 14-membered heterocyclyl, and 5- to 6-membered heteroaryl;
B is selected from azetidinyl, piperidyl, piperazinyl, 2-azaspiro[3.3]heptanyl, 2,6-diazaspiro[3.3]heptanyl, 2-azaspiro[3.4]octanyl, 6-azaspiro[3.4]octanyl, 2,7-diazaspiro[3.4]octanyl, 2,7-diazaspiro[3.5]nonanyl, 2-azaspiro[3.5]nonanyl, 7-azaspiro[3.5]nonanyl, 1,2,3,3a,4,5,6,6a-octahydrocyclopenta[c]pyrrolyl, cyclobutyl, cubanyl, spiro[3.3]heptanyl, a group 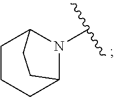 [SMILES: *N1C2CCCC1CC2]
and a group 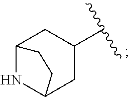 [SMILES: *C1CC2CCC(C1)N2]
C is selected from phenyl, azetidinyl, pyrrolidinyl, piperidyl, piperazinyl, morpholinyl, 4,5-dihydroisoxazolyl, 2-azabicyclo[2.1.1]hexanyl, 2-azaspiro[3.3]heptanyl, and 2,8-diazaspiro[3.5]nonanyl;
D is selected from C₆₋₁₄-aryl, C₃₋₁₀-cycloalkyl, 5-6-membered heteroaryl, and 3- to 14-membered heterocyclyl;
E is selected from 5- to 14-membered heteroaryl, 3- to 14-membered heterocyclyl, and C₃₋₁₀-cycloalkyl;
F is selected from C₃₋₁₀-cycloalkyl, 3- to 14-membered heterocyclyl, and 5- to 14-membered heteroaryl;
L is selected from a covalent bond, —CHR⁶—, —O—, —NR¹¹—, —NHCH₂—, —CH₂NR¹¹—, —OCH₂—, —CH₂O—, —CH₂OCH₂—, —CH₂CH₂—, —CF₂CH₂—, —CH₂CF₂—, —CH═CH—, —C≡C—, —SO₂—, —SO₂NH—, —SO₂NHCH₂—, —C(O)NH—, —NHC(O)—, —CH₂NHC(O)—, and 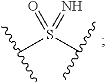 [SMILES: *S(*)(=N)=O]
L¹ is selected from a covalent bond, carbonyl, —CH₂—, —CH₂O—, —NHCH₂—, CH₂NH—, —NR¹¹—, —C(O)—NH—NH—C(O)—CH₂—, —SO₂—, and —O—;
L² is selected from a covalent bond and —SO₂—;
L³ is selected from a covalent bond, carbonyl, and —CH₂—; and
X is selected from O and NH.

10. The compound of formula (I) according to claim 9, or a pharmaceutically acceptable salt thereof, wherein:
R⁵ is a group 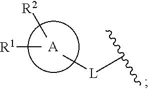
R¹ is selected from halogen, C₁₋₆-alkyl, halo-C₁₋₆-alkyl, and a group 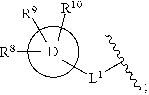
R² is selected from hydrogen, halogen, and halo-C₁₋₆-alkyl;
R^{2a} is hydrogen;
R³ is hydrogen;
R⁴ is selected from a group 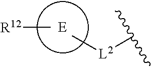
and carbamoyl;
R^{4a} is hydrogen;
R⁸ and R⁹ are each independently selected from halogen and halo-C₁₋₆-alkyl;
R¹⁰ is hydrogen;
R¹² is selected from hydrogen and oxo;
A is selected from C₆₋₁₄-aryl and C₃₋₁₀-cycloalkyl;
B is selected from spiro[3.3]heptan-2-yl, azetidinyl, and 7-azaspiro[3.5]nonan-7-yl;
C is pyrrolidinyl;
D is selected from C₆₋₁₄-aryl and C₃₋₁₀-cycloalkyl;
E is selected from 5-membered heteroaryl and 5-membered heterocyclyl;
L is selected from a covalent bond, —O— and —CH₂O—;
L¹ is a covalent bond; and
L² is a covalent bond.

11. The compound of formula (I) according to claim 10, or a pharmaceutically acceptable salt thereof, wherein:
R⁵ is a group 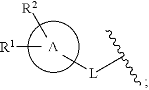
R¹ is selected from fluoro, chloro, tert-butyl, CHF₂, CF₃, and a group 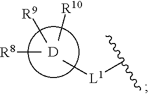
R² is selected from hydrogen, fluoro, chloro, and CF₃;
R^{2a} is hydrogen;
R³ is hydrogen;
R⁴ is selected from a group 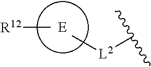
and carbamoyl;
R^{4a} is hydrogen;
R⁸ and R⁹ are each independently selected from fluoro, chloro and CF₃;
R¹⁰ is hydrogen;
R¹² is selected from hydrogen and oxo;
A is selected from phenyl and cyclopropyl;
B is selected from spiro[3.3]heptan-2-yl, azetidinyl, and 7-azaspiro[3.5]nonan-7-yl;
C is pyrrolidinyl;
D is selected from phenyl and cyclopropyl;
E is selected from imidazolyl, pyrazolyl, triazolyl, and oxazolidinyl;
L is selected from a covalent bond, —O— and —CH₂O—;
L¹ is a covalent bond; and
L² is a covalent bond.

12. A compound of formula (I) according to claim 1, or a pharmaceutically acceptable salt thereof, selected from:
[3-[[2-Fluoro-6-(trifluoromethyl)phenyl]methoxy]azetidin-1-yl]-[3-(1H-pyrazol-5-yl)pyrrolidin-1-yl]methanone;
[3-[[2-Fluoro-4-(trifluoromethyl)phenyl]methoxy]azetidin-1-yl]-[3-(1H-pyrazol-5-yl)pyrrolidin-1-yl]methanone;
4-[1-[3-[[2-Fluoro-4-(trifluoromethyl)phenyl]methoxy]azetidine-1-carbonyl]pyrrolidin-3-yl]oxazolidin-2-one;
(+)- or (−)-[3-[[2-Fluoro-4-(trifluoromethyl)phenyl]methoxy]azetidin-1-yl]-[3-(1H-pyrazol-5-yl)pyrrolidin-1-yl]methanone;
(−)- or (+)-[3-[[2-Fluoro-4-(trifluoromethyl)phenyl]methoxy]azetidin-1-yl]-[3-(1H-pyrazol-5-yl)pyrrolidin-1-yl]methanone;
[3-[[2-Fluoro-4-(trifluoromethyl)phenyl]methoxy]azetidin-1-yl]-[3-(4-methyl-1H-pyrazol-5-yl)azetidin-1-yl]methanone;
[3-[[2-Fluoro-4-(trifluoromethyl)phenyl]methoxy]azetidin-1-yl]-[3-(1H-pyrazol-5-yl)azetidin-1-yl]methanone;
[4-[(2-Chloro-4-fluoro-phenyl)methoxy]-1-piperidyl]-[3-(4-methyl-1H-pyrazol-5-yl)azetidin-1-yl]methanone;
[4-[(4-Chloro-2-fluoro-phenoxy)methyl]-1-piperidyl]-[3-(1H-pyrazol-5-yl)pyrrolidin-1-yl]methanone;
[3-[[2-Fluoro-4-(trifluoromethyl)phenyl]methoxy]azetidin-1-yl]-[3-(1H-pyrazol-5-yl)-1-piperidyl]methanone;
[6-(2-Chloro-4-fluoro-phenoxy)-2-azaspiro[3.3]heptan-2-yl]-[3-(1H-pyrazol-5-yl)pyrrolidin-1-yl]methanone;
[3-(1H-Pyrazol-5-yl)pyrrolidin-1-yl]-[[4-[[4-(trifluoromethyl)phenyl]methyl]-1-piperidyl]methanone;
[3-[2-Fluoro-4-(trifluoromethyl)phenyl]methoxy]azetidin-1-yl]-[3-(2-methylpyrazol-3-yl)pyrrolidin-1-yl]methanone;
[3-[[2-Fluoro-4-(trifluoromethyl)phenyl]methoxy]azetidin-1-yl]-[3-(1H-pyrazol-5-yl)phenyl]methanone;
[3-[[2-Fluoro-4-(pentafluoro-26-sulfanyl)phenyl]methoxy]azetidin-1-yl]-[3-(1H-pyrazol-5-yl)pyrrolidin-1-yl]methanone;
(+)- or (−)-[3-[[2-Fluoro-6-(trifluoromethyl)phenyl]methoxy]azetidin-1-yl]-[3-(1H-pyrazol-5-yl)pyrrolidin-1-yl]methanone;
(−)- or (+)-[3-[[2-Fluoro-6-(trifluoromethyl)phenyl]methoxy]azetidin-1-yl]-[3-(1H-pyrazol-5-yl)pyrrolidin-1-yl]methanone;
[3-[4-(2,4-Dichlorophenyl)phenyl]azetidin-1-yl]-[3-(1H-pyrazol-5-yl)pyrrolidin-1-yl]methanone;
[3-[[2-Fluoro-4-(trifluoromethyl)phenyl]methoxy]azetidin-1-yl]-[3-(1H-1,2,4-triazol-5-yl)pyrrolidin-1-yl]methanone;
6-[1-[3-[[2-Fluoro-4-(trifluoromethyl)phenyl]methoxy]azetidine-1-carbonyl]pyrrolidin-3-yl]-1H-pyridin-2-one;
6-[1-[3-[[2-Fluoro-4-(trifluoromethyl)phenyl]methoxy]azetidine-1-carbonyl]pyrrolidin-3-yl]-1H-pyrazin-2-one;
[3-[[2-Fluoro-4-(trifluoromethyl)phenyl]methoxy]azetidin-1-yl]-[3-(4-isopropyl-1H-pyrazol-3-yl)pyrrolidin-1-yl]methanone;
4-[1-[3-[[2-Fluoro-4-(trifluoromethyl)phenyl]methoxy]azetidine-1-carbonyl]pyrrolidin-3-yl]oxazolidin-2-one;
Ethyl 5-[1-[3-[[2-fluoro-4-(trifluoromethyl)phenyl]methoxy]azetidine-1-carbonyl]pyrrolidin-3-yl]-1H-pyrazole-4-carboxylate;
5-[1-[3-[[2-Fluoro-4-(trifluoromethyl)phenyl]methoxy]azetidine-1-carbonyl]pyrrolidin-3-yl]pyrrolidin-2-one;
[3-[[2-Fluoro-4-(trifluoromethyl)phenyl]methoxy]azetidin-1-yl]-[(3R)-3-(1H-1,2,4-triazol-5-yl)pyrrolidin-1-yl]methanone;
[3-[[2-Fluoro-4-(trifluoromethyl)phenyl]methoxy]azetidin-1-yl]-[(3S)-3-(1H-1,2,4-triazol-5-yl)pyrrolidin-1-yl]methanone;
1-[3-[[2-Fluoro-4-(trifluoromethyl)phenyl]methoxy]azetidine-1-carbonyl]pyrrolidine-3-carboxamide;
[3-[[2-Fluoro-4-(trifluoromethyl)phenyl]methoxy]azetidin-1-yl]-[3-(1H-triazol-5-yl)pyrrolidin-1-yl]methanone;
[3-(1H-1,2,4-Triazol-5-yl)pyrrolidin-1-yl]-[3-[4-[1-(trifluoromethyl)cyclopropyl]phenyl]azetidin-1-yl]methanone;
[3-(4-tert-Butylphenyl)azetidin-1-yl]-[3-(1H-1,2,4-triazol-5-yl)pyrrolidin-1-yl]methanone;
[3-[[2-Fluoro-4-(trifluoromethyl)phenyl]methoxy]azetidin-1-yl]-[3-(1,2,4-triazol-1-yl)pyrrolidin-1-yl]methanone;
[3-[[2-Fluoro-4-(trifluoromethyl)phenyl]methoxy]azetidin-1-yl]-(3-imidazol-1-ylpyrrolidin-1-yl)methanone;
[3-[[2,4-bis(Trifluoromethyl)phenyl]methoxy]azetidin-1-yl]-[3-(1H-1,2,4-triazol-5-yl)pyrrolidin-1-yl]methanone;
(+)- or (−)-[2-(2-Chloro-4-fluoro-phenoxy)-7-azaspiro[3.5]nonan-7-yl]-[(3R)-3-(1H-1,2,4-triazol-5-yl)pyrrolidin-1-yl]methanone;
(−)- or (+)-[2-(2-Chloro-4-fluoro-phenoxy)-7-azaspiro[3.5]nonan-7-yl]-[(3S)-3-(1H-1,2,4-triazol-5-yl)pyrrolidin-1-yl]methanone;
(+)- or (−)-[3-(1H-Pyrazol-5-yl)pyrrolidin-1-yl]-[3-(4-tetrahydropyran-4-ylphenyl)azetidin-1-yl]methanone;
(−)- or (+)-[3-(1H-Pyrazol-5-yl)pyrrolidin-1-yl]-[3-(4-tetrahydropyran-4-ylphenyl)azetidin-1-yl]methanone;
(+)- or (−)-[3-(4-Isopropoxyphenyl)azetidin-1-yl]-[3-(1H-pyrazol-5-yl)pyrrolidin-1-yl]methanone;
(−)- or (+)-[3-(4-Isopropoxyphenyl)azetidin-1-yl]-[(3S)-3-(1H-pyrazol-5-yl)pyrrolidin-1-yl]methanone;
(+)- or (−)-[6-[(2,4-Difluorophenyl)methyl]-2-azaspiro[3.3]heptan-2-yl]-[3-(1H-1,2,4-triazol-5-yl)pyrrolidin-1-yl]methanone;
(−)- or (+)-[6-[(2,4-Difluorophenyl)methyl]-2-azaspiro[3.3]heptan-2-yl]-[3-(1H-1,2,4-triazol-5-yl)pyrrolidin-1-yl]methanone;
[3-[[2-Fluoro-4-(trifluoromethyl)phenyl]methoxy]azetidin-1-yl]-[3-(1H-imidazol-5-yl)pyrrolidin-1-yl]methanone;
(+)- or (−)-[2-[2-Fluoro-4-(trifluoromethyl) phenoxy]-7-azaspiro[3.5]nonan-7-yl]-[3-(1H-1,2,4-triazol-5-yl)pyrrolidin-1-yl]methanone;
(−)- or (+)-[2-[2-Fluoro-4-(trifluoromethyl) phenoxy]-7-azaspiro[3.5]nonan-7-yl]-[3-(1H-1,2,4-triazol-5-yl)pyrrolidin-1-yl]methanone;
[3-[[2-Fluoro-4-(trifluoromethyl)phenyl]methoxy]azetidin-1-yl]-[3-(1H-imidazol-2-yl)pyrrolidin-1-yl]methanone;
[3-[[2-Fluoro-4-(trifluoromethyl)phenyl]methoxy]azetidin-1-yl]-[(3S)-3-(1H-triazol-5-yl)pyrrolidin-1-yl]methanone;
Sodium [3-[[2-fluoro-4-(trifluoromethyl)phenyl]methoxy]azetidin-1-yl]-[rac-(3S)-3-(1H-triazol-5-yl)pyrrolidin-1-yl]methanone;
[3-[[2-Fluoro-4-(trifluoromethyl)phenyl]methoxy]azetidin-1-yl]-[(3R)-3-(1H-triazol-5-yl)pyrrolidin-1-yl]methanone;
(+)- or (−)-[3-[[2-Fluoro-4-(trifluoromethyl)phenyl]methoxy]azetidin-1-yl]-[3-imidazol-1-ylpyrrolidin-1-yl]methanone;
(−)- or (+)-[3-[[2-Fluoro-4-(trifluoromethyl)phenyl]methoxy]azetidin-1-yl]-[3-imidazol-1-ylpyrrolidin-1-yl]methanone;
[3-[[3-Chloro-4-(trifluoromethyl)phenyl]methoxy]azetidin-1-yl]-[3-(1H-1,2,4-triazol-5-yl)pyrrolidin-1-yl]methanone;
[3-[[2-Chloro-4-(trifluoromethyl)phenyl]methoxy]azetidin-1-yl]-[3-(1H-1,2,4-triazol-5-yl)pyrrolidin-1-yl]methanone;
(+)- or (−)-[3-[2-[2-Fluoro-4-(trifluoromethyl)phenyl]ethyl]azetidin-1-yl]-[3-(1H-1,2,4-triazol-5-yl)pyrrolidin-1-yl]methanone;
(−)- or (+)-[3-[2-[2-Fluoro-4-(trifluoromethyl)phenyl]ethyl]azetidin-1-yl]-[3-(1H-1,2,4-triazol-5-yl)pyrrolidin-1-yl]methanone;
(+)- or (−)-[3-(4-Propoxyphenyl)azetidin-1-yl]-[3-(1H-pyrazol-5-yl)pyrrolidin-1-yl]methanone;
(−)- or (+)-[3-(4-Propoxyphenyl)azetidin-1-yl]-[3-(1H-pyrazol-5-yl)pyrrolidin-1-yl]methanone;
(+)- or (−)-[3-[[2,4-bis(Trifluoromethyl)phenyl]methoxy]azetidin-1-yl]-[3-(1H-1,2,4-triazol-5-yl)pyrrolidin-1-yl]methanone;
(−)- or (+)-[3-[2,4-bis(Trifluoromethyl)phenyl]methoxy]azetidin-1-yl]-[3-(1H-1,2,4-triazol-5-yl)pyrrolidin-1-yl]methanone;
[3-[[2-Fluoro-4-(trifluoromethyl)phenyl]methoxy]azetidin-1-yl]-[3-(1H-tetrazol-5-yl)pyrrolidin-1-yl]methanone;
(+)- or (−)-1-[3-[[2-Fluoro-4-(trifluoromethyl)phenyl]methoxy]azetidine-1-carbonyl]-N-methyl-pyrrolidine-3-carboxamide;
(−)- or (+)-1-[3-[[2-Fluoro-4-(trifluoromethyl)phenyl]methoxy]azetidine-1-carbonyl]-N-methyl-pyrrolidine-3-carboxamide;
(+)- or (−)-1-[3-[2-Fluoro-4-(trifluoromethyl)phenyl]methoxy]azetidine-1-carbonyl]-N,N-dimethyl-pyrrolidine-3-carboxamide;
(−)- or (+)-1-[3-[[2-Fluoro-4-(trifluoromethyl)phenyl]methoxy]azetidine-1-carbonyl]-N,N-dimethyl-pyrrolidine-3-carboxamide;
(+)- or (−)-[3-[[2-Fluoro-4-(trifluoromethyl)phenyl]methoxy]azetidin-1-yl]-[3-pyrazol-1-ylpyrrolidin-1-yl]methanone;
(−)- or (+)-[3-[[2-Fluoro-4-(trifluoromethyl)phenyl]methoxy]azetidin-1-yl]-[3-pyrazol-1-ylpyrrolidin-1-yl]methanone;
(+)- or (−)-1-[3-[4-(2,4-Difluorophenyl)phenyl]azetidine-1-carbonyl]pyrrolidine-3-carboxamide;
(−)- or (+)-1-[3-[4-(2,4-Difluorophenyl)phenyl]azetidine-1-carbonyl]pyrrolidine-3-carboxamide;
[3-(1H-Pyrazol-5-yl)pyrrolidin-1-yl]-[3-(4-THF-3-ylphenyl)azetidin-1-yl]methanone;
[3-(4-tert-Butylphenyl)azetidin-1-yl]-[3-(1H-pyrazol-5-yl)pyrrolidin-1-yl]methanone;
[3-[2-[2-fluoro-4-(trifluoromethyl)phenyl]ethyl]azetidin-1-yl]-[(3S)-3-(1H-pyrazol-5-yl)pyrrolidin-1-yl]methanone;
[3-[[2-Fluoro-4-(trifluoromethyl)phenyl]methoxy]azetidin-1-yl]-[3-(triazol-1-yl)pyrrolidin-1-yl]methanone;
[6-[(2,4-difluorophenyl)methyl]-2-azaspiro[3.3]heptan-2-yl]-[(3S)-3-(1H-pyrazol-5-yl)pyrrolidin-1-yl]methanone;
[3-(1H-Pyrazol-5-yl)pyrrolidin-1-yl]-[3-(4-tetrahydropyran-3-ylphenyl)azetidin-1-yl]methanone;
[3-[[2-Fluoro-4-(trifluoromethyl)phenyl]methoxy]azetidin-1-yl]-(3-pyrrol-1-ylpyrrolidin-1-yl)methanone;
trans-[3-[[2-Fluoro-4-(trifluoromethyl)phenyl]methoxy]cyclobutyl]-[3-(1H-triazol-5-yl)pyrrolidin-1-yl]methanone;
(+)- or (−)-[3-[4-[(1R,5S)-8-Oxa-3-azabicyclo[3.2.1]octan-3-yl]phenyl]azetidin-1-yl]-[3-(1H-pyrazol-5-yl)pyrrolidin-1-yl]methanone;
(−)- or (+)-[3-[4-[(1R,5S)-8-Oxa-3-azabicyclo[3.2.1]octan-3-yl]phenyl]azetidin-1-yl]-[3-(1H-pyrazol-5-yl)pyrrolidin-1-yl]methanone;
(−)- or (+)-[3-[4-(2,4-Difluorophenyl)phenyl]azetidin-1-yl]-[3-(1H-1,2,4-triazol-5-yl)pyrrolidin-1-yl]methanone;
(+)- or (−)-[3-[4-(2,4-Difluorophenyl)phenyl]azetidin-1-yl]-[3-(1H-1,2,4-triazol-5-yl)pyrrolidin-1-yl]methanone;
[3-[4-(2,4-Difluorophenyl)phenyl]azetidin-1-yl]-[(3S)-3-(1H-pyrazol-5-yl)pyrrolidin-1-yl]methanone;
[4-[bis(4-Fluorophenyl)methyl]-1-piperidyl]-[3-(1H-1,2,4-triazol-5-yl)pyrrolidin-1-yl]methanone;
4-[1-[3-[3-Chloro-4-(trifluoromethoxy)phenyl]azetidine-1-carbonyl]pyrrolidin-3-yl]oxazolidin-2-one;
[3-[4-(2-Methylazetidin-1-yl)phenyl]azetidin-1-yl]-[(3S or 3R)-3-(1H-pyrazol-5-yl)pyrrolidin-1-yl]methanone;
[3-[4-(2-Methylazetidin-1-yl)phenyl]azetidin-1-yl]-[(3R or 3S)-3-(1H-pyrazol-5-yl)pyrrolidin-1-yl]methanone;
[3-[4-(3-Fluoropyrrolidin-1-yl)phenyl]azetidin-1-yl]-[(3R or 3S)-3-(1H-pyrazol-5-yl)pyrrolidin-1-yl]methanone;
(+)- or (−)-[3-(1H-1,2,4-Triazol-5-yl)pyrrolidin-1-yl]-[3-[4-[1-(trifluoromethyl)cyclopropyl]phenyl]azetidin-1-yl]methanone;
(−)- or (+)-[3-(1H-1,2,4-Triazol-5-yl)pyrrolidin-1-yl]-[3-[4-[1-(trifluoromethyl)cyclopropyl]phenyl]azetidin-1-yl]methanone;
(+)- or (−)-[3-[[2-Chloro-4-(trifluoromethyl)phenyl]methoxy]azetidin-1-yl]-[3-(1H-1,2,4-triazol-5-yl)pyrrolidin-1-yl]methanone;
(−)- or (+)-[3-[[2-Chloro-4-(trifluoromethyl)phenyl]methoxy]azetidin-1-yl]-[3-(1H-1,2,4-triazol-5-yl)pyrrolidin-1-yl]methanone;
[3-[4-(3-Fluoropyrrolidin-1-yl)phenyl]azetidin-1-yl]-[(3S or 3R)-3-(1H-pyrazol-5-yl)pyrrolidin-1-yl]methanone;
4-[1-[3-[4-(2,2,2-Trifluoroethyl)phenyl]azetidine-1-carbonyl]pyrrolidin-3-yl]oxazolidin-2-one;
[3-[4-(Oxetan-3-yl)phenyl]azetidin-1-yl]-[3-(1H-pyrazol-5-yl)pyrrolidin-1-yl]methanone;
(+)- or (−)-[3-[4-(Oxetan-3-yl)phenyl]azetidin-1-yl]-[3-(1H-pyrazol-5-yl)pyrrolidin-1-yl]methanone;
(−)- or (+)-[3-[4-(Oxetan-3-yl)phenyl]azetidin-1-yl]-[3-(1H-pyrazol-5-yl)pyrrolidin-1-yl]methanone;
4-[1-[3-[4-(2,4-Difluorophenyl)phenyl]azetidine-1-carbonyl]pyrrolidin-3-yl]oxazolidin-2-one;
[3-(1H-Triazol-5-yl)pyrrolidin-1-yl]-[3-[4-[1-(trifluoromethyl)cyclopropyl]phenyl]azetidin-1-yl]methanone;
[4-[bis(4-Fluorophenyl)methyl]-1-piperidyl]-[3-(1H-triazol-5-yl)pyrrolidin-1-yl]methanone;
4-[1-[3-[4-[1-(Trifluoromethyl)cyclopropyl]phenyl]azetidine-1-carbonyl]pyrrolidin-3-yl]oxazolidin-2-one;
(+)- or (−)-[3-(1H-Triazol-5-yl)pyrrolidin-1-yl]-[3-[4-[1-(trifluoromethyl)cyclopropyl]phenyl]azetidin-1-yl]methanone;
(−)- or (+)-[3-(1H-Triazol-5-yl)pyrrolidin-1-yl]-[3-[4-[1-(trifluoromethyl)cyclopropyl]phenyl]azetidin-1-yl]methanone;
(+)- or (−)-[3-(3,3-Dimethyl-2H-benzofuran-6-yl)azetidin-1-yl]-[3-(1H-pyrazol-5-yl)pyrrolidin-1-yl]methanone;
(−)- or (+)-[3-(3,3-Dimethyl-2H-benzofuran-6-yl)azetidin-1-yl]-[3-(1H-pyrazol-5-yl)pyrrolidin-1-yl]methanone;
(+)- or (−)-[4-[bis(4-Fluorophenyl)methyl]-1-piperidyl]-[3-(1H-triazol-5-yl)pyrrolidin-1-yl]methanone;
(−)- or (+)-[4-[bis(4-Fluorophenyl)methyl]-1-piperidyl]-[3-(1H-triazol-5-yl)pyrrolidin-1-yl]methanone;
[3-[[2-Chloro-4-(trifluoromethyl)phenyl]methoxy]azetidin-1-yl]-[(3S)-3-(1H-triazol-5-yl)pyrrolidin-1-yl]methanone;
[(3S)-3-(1H-Triazol-5-yl)pyrrolidin-1-yl]-[3-[4-(2,2,2-trifluoro-1,1-dimethyl-ethoxy)phenyl]azetidin-1-yl]methanone;
[3-[[2-Fluoro-4-(pentafluoro-26-sulfanyl)phenyl]methoxy]azetidin-1-yl]-[(3S)-3-(1H-triazol-5-yl)pyrrolidin-1-yl]methanone;
[6-[(2,4-Difluorophenyl)methyl]-2-azaspiro[3.3]heptan-2-yl]-[(3S)-3-(1H-triazol-5-yl)pyrrolidin-1-yl]methanone;
(+)-[4-[(2-Chloro-4-fluoro-phenoxy)methyl]-1-piperidyl]-[(3R)-3-(1H-triazol-5-yl)pyrrolidin-1-yl]methanone;
(−)-[4-[(2-Chloro-4-fluoro-phenoxy)methyl]-1-piperidyl]-[(3S)-3-(1H-triazol-5-yl)pyrrolidin-1-yl]methanone;
(+)- or (−)-[3-(1H-1,2,4-Triazol-5-yl)pyrrolidin-1-yl]-[3-[4-(2,2,2-trifluoro-1,1-dimethyl-ethoxy)phenyl]azetidin-1-yl]methanone;
(−)- or (+)-[3-(1H-1,2,4-Triazol-5-yl)pyrrolidin-1-yl]-[3-[4-(2,2,2-trifluoro-1,1-dimethyl-ethoxy)phenyl]azetidin-1-yl]methanone;
(+)- or (−)-[4-[2-Chloro-4-(trifluoromethyl)phenyl]methoxy]-1-piperidyl]-[(3R)-3-(1H-triazol-5-yl)pyrrolidin-1-yl]methanone;
(−)- or (+)-[4-[2-Chloro-4-(trifluoromethyl)phenyl]methoxy]-1-piperidyl]-[(3S)-3-(1H-triazol-5-yl)pyrrolidin-1-yl]methanone;
[(3R or 3S)-3-(1H-Triazol-5-yl)pyrrolidin-1-yl]-[3-[[4-(trifluoromethyl)phenyl]methyl]pyrrolidin-1-yl]methanone;
[3-[[2-Fluoro-4-(trifluoromethyl)phenyl]methoxy]-8-azabicyclo[3.2.1]octan-8-yl]-[(3R or 3S)-3-(1H-triazol-5-yl)pyrrolidin-1-yl]methanone;
[3-[[2-Fluoro-4-(trifluoromethyl)phenyl]methoxy]-8-azabicyclo[3.2.1]octan-8-yl]-[(3S or 3R)-3-(1H-triazol-5-yl)pyrrolidin-1-yl]methanone;
[(3aS,6aR)-5-[2-Fluoro-4-(trifluoromethyl) phenoxy]-3,3a,4,5,6,6a-hexahydro-1H-cyclopenta[c]pyrrol-2-yl]-[(3R or 3S)-3-(1H-triazol-5-yl)pyrrolidin-1-yl]methanone;
[(3aS,6aR)-5-[2-Fluoro-4-(trifluoromethyl) phenoxy]-3,3a,4,5,6,6a-hexahydro-1H-cyclopenta[c]pyrrol-2-yl]-[(3S or 3R)-3-(1H-triazol-5-yl)pyrrolidin-1-yl]methanone;
(−)-[3-[[2-Fluoro-4-(trifluoromethyl)phenyl]methoxy]-2-methyl-azetidin-1-yl]-[(3R or 3S)-3-(1H-triazol-5-yl)pyrrolidin-1-yl]methanone;
(+)-[3-[[2-Fluoro-4-(trifluoromethyl)phenyl]methoxy]-2-methyl-azetidin-1-yl]-[(3R or 3S)-3-(1H-triazol-5-yl)pyrrolidin-1-yl]methanone;
(−)-[3-[[2-Fluoro-4-(trifluoromethyl)phenyl]methoxy]-2-methyl-azetidin-1-yl]-[(3R or 3S)-3-(1H-triazol-5-yl)pyrrolidin-1-yl]methanone;
(+)-[3-[[2-Fluoro-4-(trifluoromethyl)phenyl]methoxy]-2-methyl-azetidin-1-yl]-[(3S or 3R)-3-(1H-triazol-5-yl)pyrrolidin-1-yl]methanone;
(−)-[3-[[2-Fluoro-4-(trifluoromethyl)phenyl]methoxy]-2-methyl-azetidin-1-yl]-[(3S or 3R)-3-(1H-triazol-5-yl)pyrrolidin-1-yl]methanone;
(+)-[3-[[2-Fluoro-4-(trifluoromethyl)phenyl]methoxy]-2-methyl-azetidin-1-yl]-[(3S or 3R)-3-(1H-triazol-5-yl)pyrrolidin-1-yl]methanone;
[3-[6-(2-Chlorophenoxy)-3-pyridyl]azetidin-1-yl]-[3-(tetrazol-1-yl)pyrrolidin-1-yl]methanone;
[3-[[2-Fluoro-4-(trifluoromethyl)phenyl]methoxy]azetidin-1-yl]-[(3S)-3-(1,2,4-triazol-4-yl)pyrrolidin-1-yl]methanone;
[3-[[2-Fluoro-4-(trifluoromethyl)phenyl]methoxy]azetidin-1-yl]-[(3R)-3-(1,2,4-triazol-4-yl)pyrrolidin-1-yl]methanone;
[3-(4H-1,2,4-Triazol-3-yl)pyrrolidin-1-yl]-[3-[4-[3-(2,2,2-trifluoroethoxy)azetidin-1-yl]phenyl]azetidin-1-yl]methanone;
[6-(2-Chloro-4-fluoro-phenoxy)-2-azaspiro[3.3]heptan-2-yl]-[3-(4H-1,2,4-triazol-3-yl)pyrrolidin-1-yl]methanone;
[3-(4H-1,2,4-Triazol-3-yl)pyrrolidin-1-yl]-[3-[6-[4-(trifluoromethoxy) phenoxy]-3-pyridyl]azetidin-1-yl]methanone;
[(3S)-3-(1,2,4-Triazol-4-yl)pyrrolidin-1-yl]-[3-[5-[1-(trifluoromethyl)cyclopropyl]-1,2,4-oxadiazol-3-yl]azetidin-1-yl]methanone;
[(3S)-3-(1,2,4-Triazol-4-yl)pyrrolidin-1-yl]-[3-[4-[3-(2,2,2-trifluoroethoxy)azetidin-1-yl]phenyl]azetidin-1-yl]methanone;
[6-(2-Chloro-4-fluoro-phenoxy)-2-azaspiro[3.3]heptan-2-yl]-[(3S)-3-(1,2,4-triazol-4-yl)pyrrolidin-1-yl]methanone;
[(3R)-3-(1,2,4-Triazol-4-yl)pyrrolidin-1-yl]-[6-[6-(trifluoromethyl)pyrazin-2-yl]oxy-2-azaspiro[3.3]heptan-2-yl]methanone;
[(3R)-3-(1,2,4-Triazol-4-yl)pyrrolidin-1-yl]-[3-[6-[4-(trifluoromethoxy) phenoxy]-3-pyridyl]azetidin-1-yl]methanone;
[3-[4-(4-Fluorophenoxy)phenyl]azetidin-1-yl]-[3-(tetrazol-1-yl)pyrrolidin-1-yl]methanone;
[3-(Tetrazol-1-yl)pyrrolidin-1-yl]-[6-[6-(trifluoromethyl)pyrazin-2-yl]oxy-2-azaspiro[3.3]heptan-2-yl]methanone;
[3-(Tetrazol-1-yl)pyrrolidin-1-yl]-[3-[6-[4-(trifluoromethoxy) phenoxy]-3-pyridyl]azetidin-1-yl]methanone;
(+)- or (−)-[3-(4H-1,2,4-Triazol-3-yl)pyrrolidin-1-yl]-[3-[4-[3-(2,2,2-trifluoroethoxy)azetidin-1-yl]phenyl]azetidin-1-yl]methanone;
(−)- or (+)-[3-(4H-1,2,4-Triazol-3-yl)pyrrolidin-1-yl]-[3-[4-[3-(2,2,2-trifluoroethoxy)azetidin-1-yl]phenyl]azetidin-1-yl]methanone;
(+)- or (−)-[6-(2-Chloro-4-fluoro-phenoxy)-2-azaspiro[3.3]heptan-2-yl]-[3-(4H-1,2,4-triazol-3-yl)pyrrolidin-1-yl]methanone;
(−)- or (+)-[6-(2-Chloro-4-fluoro-phenoxy)-2-azaspiro[3.3]heptan-2-yl]-[3-(4H-1,2,4-triazol-3-yl)pyrrolidin-1-yl]methanone;
(+)- or (−)-[3-(4H-1,2,4-Triazol-3-yl)pyrrolidin-1-yl]-[3-[6-[4-(trifluoromethoxy) phenoxy]-3-pyridyl]azetidin-1-yl]methanone;
(−)- or (+)-[3-(4H-1,2,4-Triazol-3-yl)pyrrolidin-1-yl]-[3-[6-[4-(trifluoromethoxy) phenoxy]-3-pyridyl]azetidin-1-yl]methanone;
(+)- or (−)-[3-[4-(4-Fluorophenoxy)phenyl]azetidin-1-yl]-[3-(tetrazol-1-yl)pyrrolidin-1-yl]methanone;
(−)- or (+)-[3-[4-(4-Fluorophenoxy)phenyl]azetidin-1-yl]-[3-(tetrazol-1-yl)pyrrolidin-1-yl]methanone;
(+)- or (−)-[3-(Tetrazol-1-yl)pyrrolidin-1-yl]-[6-[6-(trifluoromethyl)pyrazin-2-yl]oxy-2-azaspiro[3.3]heptan-2-yl]methanone;
(−)- or (+)-[3-(Tetrazol-1-yl)pyrrolidin-1-yl]-[6-[6-(trifluoromethyl)pyrazin-2-yl]oxy-2-azaspiro[3.3]heptan-2-yl]methanone;
(+)- or (−)-[3-(Tetrazol-1-yl)pyrrolidin-1-yl]-[3-[6-[4-(trifluoromethoxy) phenoxy]-3-pyridyl]azetidin-1-yl]methanone;
(−)- or (+)-[3-(Tetrazol-1-yl)pyrrolidin-1-yl]-[3-[6-[4-(trifluoromethoxy) phenoxy]-3-pyridyl]azetidin-1-yl]methanone;
[(3R)-3-(1H-Triazol-5-yl)pyrrolidin-1-yl]-[2-(3,3,3-trifluoropropoxy)spiro[3.3]heptan-6-yl]methanone;
[(3R)-3-(1H-Triazol-5-yl)pyrrolidin-1-yl]-[3-[5-[1-(trifluoromethyl)cyclopropyl]-1,2,4-oxadiazol-3-yl]azetidin-1-yl]methanone;
[3-[4-(4-Fluorophenoxy)phenyl]azetidin-1-yl]-[(3R)-3-(1H-triazol-5-yl)pyrrolidin-1-yl]methanone;
[(3R)-3-(1H-Triazol-5-yl)pyrrolidin-1-yl]-[3-[4-[3-(2,2,2-trifluoroethoxy)azetidin-1-yl]phenyl]azetidin-1-yl]methanone;
[6-(2-Chloro-4-fluoro-phenoxy)-2-azaspiro[3.3]heptan-2-yl]-[(3R)-3-(1H-triazol-5-yl)pyrrolidin-1-yl]methanone;
[(3R)-3-(1H-Triazol-5-yl)pyrrolidin-1-yl]-[3-[6-[4-(trifluoromethoxy) phenoxy]-3-pyridyl]azetidin-1-yl]methanone;
[(3R)-3-(1H-Triazol-5-yl)pyrrolidin-1-yl]-[3-[6-[3-(trifluoromethyl)pyrrolidin-1-yl]-3-pyridyl]azetidin-1-yl]methanone;
[3-[2-(3-Chlorophenyl) ethynyl]azetidin-1-yl]-[(3R)-3-(1H-triazol-5-yl)pyrrolidin-1-yl]methanone;
[3-[2-[2-(Difluoromethyl)phenyl]ethynyl]azetidin-1-yl]-[(3R)-3-(1H-triazol-5-yl)pyrrolidin-1-yl]methanone;
[4-(3-Cyclopropyl-1,2,4-oxadiazol-5-yl) piperazin-1-yl]-[rac-(3S)-3-(1H-triazol-5-yl)pyrrolidin-1-yl]methanone;
[(3S)-3-[3-Chloro-5-(2,2,2-trifluoroethoxy) phenoxy]pyrrolidin-1-yl]-[(3S or 3R)-3-(1H-triazol-5-yl)pyrrolidin-1-yl]methanone;
[(3S)-3-[3-chloro-5-(2,2,2-trifluoroethoxy) phenoxy]pyrrolidin-1-yl]-[(3R or 3S)-3-(1H-triazol-5-yl)pyrrolidin-1-yl]methanone;
[(3R)-3-[3-Chloro-5-(2,2,2-trifluoroethoxy) phenoxy]pyrrolidin-1-yl]-[(3S or 3R)-3-(1H-triazol-5-yl)pyrrolidin-1-yl]methanone;
[(3R)-3-[3-Chloro-5-(2,2,2-trifluoroethoxy) phenoxy]pyrrolidin-1-yl]-[(3R or 3S)-3-(1H-triazol-5-yl)pyrrolidin-1-yl]methanone;
[(3S)-3-(1H-Triazol-5-yl)pyrrolidin-1-yl]-[2-(3,3,3-trifluoropropoxy)spiro[3.3]heptan-6-yl]methanone;
[2-[1-(Difluoromethyl)cyclopropyl]methoxy]spiro[3.3]heptan-6-yl]-[(3S)-3-(1H-triazol-5-yl)pyrrolidin-1-yl]methanone;
[8-(3-Cyclopropyl-1,2,4-oxadiazol-5-yl)-8-azabicyclo[3.2.1]octan-3-yl]-[(3S)-3-(1H-triazol-5-yl)pyrrolidin-1-yl]methanone;
[3-[4-(4-Chloro-2-methylsulfonyl-phenyl)phenyl]azetidin-1-yl]-[3-(triazol-1-yl)pyrrolidin-1-yl]methanone;
[3-[4-[3-(2,2-Dimethylpropyl)triazol-4-yl]phenyl]azetidin-1-yl]-[3-(triazol-1-yl)pyrrolidin-1-yl]methanone;
[3-(Triazol-1-yl)pyrrolidin-1-yl]-[3-[4-[1-(trifluoromethyl)cyclopropyl]phenyl]azetidin-1-yl]methanone;
[3-(2-Chloro-4-isopropylsulfonyl-phenyl)azetidin-1-yl]-[(3S)-3-(1H-triazol-5-yl)pyrrolidin-1-yl]methanone;
[3-(4-tert-Butylsulfonyl-2-chloro-phenyl)azetidin-1-yl]-[(3S)-3-(1H-triazol-5-yl)pyrrolidin-1-yl]methanone;
[3-[5-(2-Chlorophenoxy)pyrazin-2-yl]azetidin-1-yl]-[(3S)-3-(1H-triazol-5-yl)pyrrolidin-1-yl]methanone;
[(3S)-3-(1H-Triazol-5-yl)pyrrolidin-1-yl]-[6-[6-(trifluoromethyl)pyrazin-2-yl]oxy-2-azaspiro[3.3]heptan-2-yl]methanone;
[3-[4-(4-Chloro-2-methylsulfonyl-phenyl)phenyl]azetidin-1-yl]-[(3S)-3-(1H-triazol-5-yl)pyrrolidin-1-yl]methanone;
[(3S)-3-(1H-Triazol-5-yl)pyrrolidin-1-yl]-[3-[5-[1-(trifluoromethyl)cyclopropyl]-1,2,4-oxadiazol-3-yl]azetidin-1-yl]methanone;
[3-[6-(2,4-Dichlorophenoxy)-3-pyridyl]azetidin-1-yl]-[(3S)-3-(1H-triazol-5-yl)pyrrolidin-1-yl]methanone;
[3-[4-(4-Fluorophenoxy)phenyl]azetidin-1-yl]-[(3S)-3-(1H-triazol-5-yl)pyrrolidin-1-yl]methanone;
[3-(2-Chloro-4-isobutylsulfonyl-phenyl)azetidin-1-yl]-[(3S)-3-(1H-triazol-5-yl)pyrrolidin-1-yl]methanone;
[3-[4-(Benzenesulfonyl)phenyl]azetidin-1-yl]-[(3S)-3-(1H-triazol-5-yl)pyrrolidin-1-yl]methanone;
[3-(2-Chloro-4-propylsulfonyl-phenyl)azetidin-1-yl]-[(3S)-3-(1H-triazol-5-yl)pyrrolidin-1-yl]methanone;
[3-[4-(2-Chloro-4-methylsulfonyl-phenyl)phenyl]azetidin-1-yl]-[(3S)-3-(1H-triazol-5-yl)pyrrolidin-1-yl]methanone;
[3-(4-tert-Butylphenyl)azetidin-1-yl]-[(3S)-3-(1H-triazol-5-yl)pyrrolidin-1-yl]methanone;
1-[3-[4-(2-Chloro-4-methylsulfonyl-phenyl)phenyl]azetidine-1-carbonyl]pyrrolidine-3-carboxamide;
[3-(5-tert-Butyl-2-pyridyl)azetidin-1-yl]-[(3S)-3-(1H-triazol-5-yl)pyrrolidin-1-yl]methanone;
1-[3-[4-(2-Chloro-4-methylsulfonyl-phenyl)phenyl]azetidine-1-carbonyl]pyrrolidine-3-carboxylic acid;
1-[3-[4-(2,4-Difluorophenyl)phenyl]azetidine-1-carbonyl]pyrrolidine-3-carboxylic acid;
2-[4-[1-[(3S)-3-(1H-Triazol-5-yl)pyrrolidine-1-carbonyl]azetidin-3-yl]phenyl]sulfonylacetonitrile;
[3-[4-(N-Methylanilino)phenyl]azetidin-1-yl]-[(3S)-3-(1H-triazol-5-yl)pyrrolidin-1-yl]methanone;
[(3S)-3-(1H-Triazol-5-yl)pyrrolidin-1-yl]-[3-[4-(trifluoromethylsulfonyl)phenyl]azetidin-1-yl]methanone;
[3-(4-Cyclohexylsulfonylphenyl)azetidin-1-yl]-[(3S)-3-(1H-triazol-5-yl)pyrrolidin-1-yl]methanone;
1-[5-[1-[(3S)-3-(1H-Triazol-5-yl)pyrrolidine-1-carbonyl]azetidin-3-yl]-2-pyridyl]cyclobutanecarbonitrile;
[6-[(2,4-Difluorophenyl)methyl]-2,6-diazaspiro[3.3]heptan-2-yl]-[(3S)-3-(1H-triazol-5-yl)pyrrolidin-1-yl]methanone;
[6-[[4-Fluoro-2-(trifluoromethyl)phenyl]methyl]-2,6-diazaspiro[3.3]heptan-2-yl]-[(3S)-3-(1H-triazol-5-yl)pyrrolidin-1-yl]methanone;
[3-[4-[N-(Cyclopropylmethyl)anilino]phenyl]azetidin-1-yl]-[(3S)-3-(1H-triazol-5-yl)pyrrolidin-1-yl]methanone;
1-[3-[4-(4-Chloro-2-methylsulfonyl-phenyl)phenyl]azetidine-1-carbonyl]pyrrolidine-3-carboxamide;
[3-[4-(N-Phenylanilino)phenyl]azetidin-1-yl]-[(3S)-3-(1H-triazol-5-yl)pyrrolidin-1-yl]methanone;
[3-(6-tert-Butyl-3-pyridyl)azetidin-1-yl]-[(3S)-3-(1H-triazol-5-yl)pyrrolidin-1-yl]methanone;
[3-[6-[(5-Methoxy-2-pyridyl)-methyl-amino]-3-pyridyl]azetidin-1-yl]-[(3S)-3-(1H-triazol-5-yl)pyrrolidin-1-yl]methanone;
[3-[6-[(5-Ethyl-2-pyridyl)-methyl-amino]-3-pyridyl]azetidin-1-yl]-[(3S)-3-(1H-triazol-5-yl)pyrrolidin-1-yl]methanone;
[3-[6-[(5-Cyclopropyl-2-pyridyl)-methyl-amino]-3-pyridyl]azetidin-1-yl]-[(3S)-3-(1H-triazol-5-yl)pyrrolidin-1-yl]methanone;
[3-[4-(N-Methylanilino)phenyl]azetidin-1-yl]-[3-(1H-1,2,4-triazol-5-yl)pyrrolidin-1-yl]methanone;
[3-[6-(N-Methylanilino)-3-pyridyl]azetidin-1-yl]-[(3S)-3-(1H-triazol-5-yl)pyrrolidin-1-yl]methanone;
[3-[6-(4-Isopropyl-N-methyl-anilino)-3-pyridyl]azetidin-1-yl]-[(3S)-3-(1H-triazol-5-yl)pyrrolidin-1-yl]methanone;
[3-[6-[(3-Ethyl-2-pyridyl)-methyl-amino]-3-pyridyl]azetidin-1-yl]-[(3S)-3-(1H-triazol-5-yl)pyrrolidin-1-yl]methanone;
[3-[6-[N-(Cyclopropylmethyl)anilino]-3-pyridyl]azetidin-1-yl]-[(3S)-3-(1H-triazol-5-yl)pyrrolidin-1-yl]methanone;
(3S)-1-[3-[4-(4-Chloro-2-methylsulfonyl-phenyl)phenyl]azetidine-1-carbonyl]pyrrolidine-3-carboxamide;
(3R)-1-[3-[4-(4-Chloro-2-methylsulfonyl-phenyl)phenyl]azetidine-1-carbonyl]pyrrolidine-3-carboxamide;
[3-[6-[N-(Cyclopropylmethyl)anilino]-3-pyridyl]azetidin-1-yl]-[3-(1H-tetrazol-5-yl)pyrrolidin-1-yl]methanone;
[3-(1H-Tetrazol-5-yl)pyrrolidin-1-yl]-[3-[4-(2,2,2-trifluoro-1,1-dimethyl-ethoxy)phenyl]azetidin-1-yl]methanone;
[2-(2-Methoxyphenyl) sulfonyl-2,6-diazaspiro[3.3]heptan-6-yl]-[(3S)-3-(1H-triazol-5-yl)pyrrolidin-1-yl]methanone;
[2-[4-Fluoro-2-(trifluoromethyl)phenyl]sulfonyl-2,6-diazaspiro[3.3]heptan-6-yl]-[(3S)-3-(1H-triazol-5-yl)pyrrolidin-1-yl]methanone;
[2-(2,2-Dimethylpropylsulfonyl)-2,6-diazaspiro[3.3]heptan-6-yl]-[(3S)-3-(1H-triazol-5-yl)pyrrolidin-1-yl]methanone;
[3-[[2-Fluoro-4-(trifluoromethyl)phenyl]methoxy]azetidin-1-yl]-[(3S)-3-(4H-1,2,4-triazol-3-yl)pyrrolidin-1-yl]methanone;
[2-[4-Fluoro-2-(trifluoromethyl)phenyl]sulfonyl-2,6-diazaspiro[3.3]heptan-6-yl]-[(3R)-3-(4H-1,2,4-triazol-3-yl)pyrrolidin-1-yl]methanone;
[2-[4-Fluoro-2-(trifluoromethyl)phenyl]sulfonyl-2,6-diazaspiro[3.3]heptan-6-yl]-[(3S)-3-(4H-1,2,4-triazol-3-yl)pyrrolidin-1-yl]methanone;
[3-[6-[N-(Cyclopropylmethyl)anilino]-3-pyridyl]azetidin-1-yl]-[(3S)-3-(1H-tetrazol-5-yl)pyrrolidin-1-yl]methanone;
[3-[6-[N-(Cyclopropylmethyl)anilino]-3-pyridyl]azetidin-1-yl]-[(3R)-3-(1H-tetrazol-5-yl)pyrrolidin-1-yl]methanone;
[3-[4-(4-Fluorophenoxy)phenyl]azetidin-1-yl]-[3-(1H-tetrazol-5-yl)pyrrolidin-1-yl]methanone;
1-[3-[6-[N-(Cyclopropylmethyl)anilino]-3-pyridyl]azetidine-1-carbonyl]pyrrolidine-3-carboxamide;
[3-[4-(4-Fluorophenoxy)phenyl]azetidin-1-yl]-[(3R)-3-(1H-tetrazol-5-yl)pyrrolidin-1-yl]methanone;
[3-[4-(4-Fluorophenoxy)phenyl]azetidin-1-yl]-[(3S)-3-(1H-tetrazol-5-yl)pyrrolidin-1-yl]methanone;
[(3S)-3-(1,2,4-Triazol-4-yl)pyrrolidin-1-yl]-[3-[4-[4-(trifluoromethyl)pyrimidin-2-yl]oxyphenyl]azetidin-1-yl]methanone;
[3-[4-(4-Chloro-2-methylsulfonyl-phenyl)phenyl]azetidin-1-yl]-[3-(1H-tetrazol-5-yl)pyrrolidin-1-yl]methanone;
[(3S)-3-(1,2,4-Triazol-4-yl)pyrrolidin-1-yl]-[6-[6-(trifluoromethyl)pyrazin-2-yl]oxy-2-azaspiro[3.3]heptan-2-yl]methanone;
[(3S)-3-(1,2,4-Triazol-4-yl)pyrrolidin-1-yl]-[6-[1-(trifluoromethyl)cyclopropyl]methoxy]-2-azaspiro[3.3]heptan-2-yl]methanone;
(3S)-1-[3-[6-[N-(Cyclopropylmethyl)anilino]-3-pyridyl]azetidine-1-carbonyl]pyrrolidine-3-carboxamide;
(3R)-1-[3-[6-[N-(Cyclopropylmethyl)anilino]-3-pyridyl]azetidine-1-carbonyl]pyrrolidine-3-carboxamide;
[6-[[4-Fluoro-2-(trifluoromethyl)phenyl]methyl]-2,6-diazaspiro[3.3]heptan-2-yl]-[(3S)-3-(4H-1,2,4-triazol-3-yl)pyrrolidin-1-yl]methanone;
[(3S)-3-(4H-1,2,4-Triazol-3-yl)pyrrolidin-1-yl]-[3-[4-[3-(trifluoromethyl)azetidin-1-yl]phenyl]azetidin-1-yl]methanone;
1-[3-[4-(Benzenesulfonyl)phenyl]azetidine-1-carbonyl]pyrrolidine-3-carboxamide;
[2-[[2-Fluoro-4-(trifluoromethyl)phenyl]methyl]-2,7-diazaspiro[3.5]nonan-7-yl]-[3-(4H-1,2,4-triazol-3-yl)pyrrolidin-1-yl]methanone;
1-[3-[4-[3-(2,2,2-Trifluoroethoxy)azetidin-1-yl]phenyl]azetidine-1-carbonyl]pyrrolidine-3-carboxamide;
[2-[(2-Chloro-4-fluoro-phenyl)methyl]-2,7-diazaspiro[3.5]nonan-7-yl]-[3-(4H-1,2,4-triazol-3-yl)pyrrolidin-1-yl]methanone;
[3-[(2-Fluoro-4-methylsulfonyl-phenyl)methoxy]azetidin-1-yl]-[3-(4H-1,2,4-triazol-3-yl)pyrrolidin-1-yl]methanone;
1-[3-[4-[1-(Trifluoromethyl)cyclopropyl]phenyl]azetidine-1-carbonyl]pyrrolidine-3-carboxamide;
1-[3-[4-[3-(Trifluoromethyl)azetidin-1-yl]phenyl]azetidine-1-carbonyl]pyrrolidine-3-carboxamide;
[3-[4-(4-Chloro-2-methylsulfonyl-phenyl)phenyl]azetidin-1-yl]-[(3R)-3-(1H-tetrazol-5-yl)pyrrolidin-1-yl]methanone;
[3-[4-(4-Chloro-2-methylsulfonyl-phenyl)phenyl]azetidin-1-yl]-[(3S)-3-(1H-tetrazol-5-yl)pyrrolidin-1-yl]methanone;
[3-[4-(4-Chloro-2-methylsulfonyl-phenyl)phenyl]azetidin-1-yl]-[(3S)-3-(1,2,4-triazol-4-yl)pyrrolidin-1-yl]methanone;
[6-[[2-Fluoro-4-(trifluoromethyl)phenyl]methyl]-2,6-diazaspiro[3.3]heptan-2-yl]-[3-(4H-1,2,4-triazol-3-yl)pyrrolidin-1-yl]methanone;
[3-(4H-1,2,4-Triazol-3-yl)pyrrolidin-1-yl]-[3-[[6-(trifluoromethyl)-3-pyridyl]methoxy]azetidin-1-yl]methanone;
[3-(4H-1,2,4-Triazol-3-yl)pyrrolidin-1-yl]-[3-[5-(trifluoromethyl)-3-pyridyl]methoxy]azetidin-1-yl]methanone;
[3-(4H-1,2,4-Triazol-3-yl)pyrrolidin-1-yl]-[6-[[5-(trifluoromethyl)-3-pyridyl]oxy]-2-azaspiro[3.3]heptan-2-yl]methanone;
[6-[(5-Chloro-3-pyridyl)oxy]-2-azaspiro[3.3]heptan-2-yl]-[3-(4H-1,2,4-triazol-3-yl)pyrrolidin-1-yl]methanone;
[6-[(6-Chloro-3-pyridyl)oxy]-2-azaspiro[3.3]heptan-2-yl]-[3-(4H-1,2,4-triazol-3-yl)pyrrolidin-1-yl]methanone;
(3S)-1-[3-[4-[3-(2,2,2-Trifluoroethoxy)azetidin-1-yl]phenyl]azetidine-1-carbonyl]pyrrolidine-3-carboxamide;
(3R)-1-[3-[4-[3-(2,2,2-Trifluoroethoxy)azetidin-1-yl]phenyl]azetidine-1-carbonyl]pyrrolidine-3-carboxamide;
(3S)-1-[3-[4-(Benzenesulfonyl)phenyl]azetidine-1-carbonyl]pyrrolidine-3-carboxamide;
(3R)-1-[3-[4-(Benzenesulfonyl)phenyl]azetidine-1-carbonyl]pyrrolidine-3-carboxamide;
[3-[(2-Fluoro-4-methylsulfonyl-phenyl)methoxy]azetidin-1-yl]-[(3S)-3-(1H-triazol-5-yl)pyrrolidin-1-yl]methanone;
2-Cyclopropyl-6-[1-[(3S)-3-(1,2,4-triazol-4-yl)pyrrolidine-1-carbonyl]azetidin-3-yl]oxy-benzonitrile;
(3S)-1-[3-[[2-Fluoro-4-(trifluoromethyl)phenyl]methoxy]azetidine-1-carbonyl]pyrrolidine-3-carboxamide;
(3S)-1-[2-[2-(Trifluoromethyl)phenyl]sulfonyl-2,6-diazaspiro[3.3]heptane-6-carbonyl]pyrrolidine-3-carboxamide;
(3S)-1-[3-[[3-(Trifluoromethyl)phenyl]sulfonylamino]methyl]azetidine-1-carbonyl]pyrrolidine-3-carboxamide;
(3S)-1-[3-[[4-(Trifluoromethyl)phenyl]sulfonylamino]methyl]azetidine-1-carbonyl]pyrrolidine-3-carboxamide;
(3S)-1-[6-[1-(Trifluoromethyl)cyclopropyl]methoxy]-2-azaspiro[3.3]heptane-2-carbonyl]pyrrolidine-3-carboxamide;
(3S)-1-[6-[6-(Trifluoromethyl)-3-pyridyl]oxy]-2-azaspiro[3.3]heptane-2-carbonyl]pyrrolidine-3-carboxamide;
(3S)-1-[6-[5-(Trifluoromethyl)-2-pyridyl]methyl]-2,6-diazaspiro[3.3]heptane-2-carbonyl]pyrrolidine-3-carboxamide;
(3S)-1-[3-[6-(Trifluoromethyl)-3-pyridyl]methoxy]azetidine-1-carbonyl]pyrrolidine-3-carboxamide;
(3S)-1-[3-[5-(Trifluoromethyl)-3-pyridyl]methoxy]azetidine-1-carbonyl]pyrrolidine-3-carboxamide;
(3S)-1-[6-[[1-(Trifluoromethyl)cyclopropyl]amino]methyl]-2-azaspiro[3.3]heptane-2-carbonyl]pyrrolidine-3-carboxamide;
[3-[4-(3-Chloropyridazin-4-yl)oxyphenyl]azetidin-1-yl]-[(3R)-3-(tetrazol-1-yl)pyrrolidin-1-yl]methanone;
[3-[[2-Fluoro-4-(trifluoromethyl)phenyl]methylamino]azetidin-1-yl]-[(3S)-3-(1H-triazol-5-yl)pyrrolidin-1-yl]methanone;
[3-[[2-Fluoro-4-(trifluoromethyl)phenyl]methylamino]azetidin-1-yl]-[3-(4H-1,2,4-triazol-3-yl)pyrrolidin-1-yl]methanone;
[3-[(2-Fluoro-4-methylsulfonyl-phenyl)methoxy]azetidin-1-yl]-[(3S)-3-(1,2,4-triazol-4-yl)pyrrolidin-1-yl]methanone;
[(3S)-3-(1,2,4-Triazol-4-yl)pyrrolidin-1-yl]-[2-[2-(trifluoromethyl)phenyl]sulfonyl-2,6-diazaspiro[3.3]heptan-6-yl]methanone;
1-[4-[1-[(3S)-3-(1,2,4-Triazol-4-yl)pyrrolidine-1-carbonyl]azetidin-3-yl]phenyl]cyclopropanecarbonitrile;
[3-[[2-Fluoro-4-(trifluoromethyl)phenyl]methylamino]azetidin-1-yl]-[(3S)-3-(1,2,4-triazol-4-yl)pyrrolidin-1-yl]methanone;
N-[2-[(3S)-3-(1,2,4-Triazol-4-yl)pyrrolidine-1-carbonyl]-2-azaspiro[3.3]heptan-6-yl]-1-(trifluoromethyl)cyclopropanecarboxamide;
[(3S)-3-(1,2,4-Triazol-4-yl)pyrrolidin-1-yl]-[3-[4-[1-(trifluoromethyl)cyclopropyl]methylamino]phenyl]azetidin-1-yl]methanone;
(3S)-1-[3-[2-[1-(Trifluoromethyl)cyclopropyl]pyrimidin-5-yl]azetidine-1-carbonyl]pyrrolidine-3-carboxamide;
(3S)-1-[3-[6-[1-(Trifluoromethyl)cyclopropyl]-3-pyridyl]azetidine-1-carbonyl]pyrrolidine-3-carboxamide;
[3-(4H-1,2,4-Triazol-3-yl)pyrrolidin-1-yl]-[3-[2-(trifluoromethyl)pyrimidin-5-yl]methoxy]azetidin-1-yl]methanone;
1-[3-[4-(4-Chloro-2-methylsulfonyl-phenyl)phenyl]azetidine-1-carbonyl]-N-methyl-pyrrolidine-3-carboxamide;
1-[3-[4-(4-Chloro-2-methylsulfonyl-phenyl)phenyl]azetidine-1-carbonyl]-N-ethyl-pyrrolidine-3-carboxamide;
(3S)-1-[3-[4-[[1-(Trifluoromethyl)cyclopropyl]amino]phenyl]azetidine-1-carbonyl]pyrrolidine-3-carboxamide;
(3S)-1-[3-[4-[[1-(Trifluoromethyl)cyclopropyl]methylamino]phenyl]azetidine-1-carbonyl]pyrrolidine-3-carboxamide;
(3S)-1-[3-[(2-Fluoro-4-methylsulfonyl-phenyl)methoxy]azetidine-1-carbonyl]pyrrolidine-3-carboxamide;
(3S)-1-[2-(2,4-Difluorophenyl) sulfonyl-2,6-diazaspiro[3.3]heptane-6-carbonyl]pyrrolidine-3-carboxamide;
(3S)-1-[2-[3-(Trifluoromethyl)phenyl]sulfonyl-2,6-diazaspiro[3.3]heptane-6-carbonyl]pyrrolidine-3-carboxamide;
(3S)-1-[2-[3-(Trifluoromethoxy)phenyl]sulfonyl-2,6-diazaspiro[3.3]heptane-6-carbonyl]pyrrolidine-3-carboxamide;
rac-(3S)-1-[3-[5-[3-(Trifluoromethyl)pyrrolidin-1-yl]-2-pyridyl]azetidine-1-carbonyl]pyrrolidine-3-carboxamide;
(3S)-1-[3-[4-(4-Chloro-2-methylsulfonyl-phenyl)phenyl]azetidine-1-carbonyl]-N-methyl-pyrrolidine-3-carboxamide;
(3R)-1-[3-[4-(4-Chloro-2-methylsulfonyl-phenyl)phenyl]azetidine-1-carbonyl]-N-methyl-pyrrolidine-3-carboxamide;
(3S)-1-[3-[4-(4-Chloro-2-methylsulfonyl-phenyl)phenyl]azetidine-1-carbonyl]-N-ethyl-pyrrolidine-3-carboxamide;
(3R)-1-[3-[4-(4-Chloro-2-methylsulfonyl-phenyl)phenyl]azetidine-1-carbonyl]-N-ethyl-pyrrolidine-3-carboxamide;
[3-[6-(2-Chlorophenoxy)-3-pyridyl]azetidin-1-yl]-[(3R)-3-(tetrazol-1-yl)pyrrolidin-1-yl]methanone;
[3-[4-(4-Fluorophenoxy)phenyl]azetidin-1-yl]-[(3S)-3-(tetrazol-1-yl)pyrrolidin-1-yl]methanone;
[3-[5-(2-Chlorophenoxy)pyrazin-2-yl]azetidin-1-yl]-[(3S)-3-(tetrazol-1-yl)pyrrolidin-1-yl]methanone;
[(3R)-3-(Tetrazol-1-yl)pyrrolidin-1-yl]-[6-[6-(trifluoromethyl)pyrazin-2-yl]oxy-2-azaspiro[3.3]heptan-2-yl]methanone;
2-Cyclopropyl-6-[1-[(3R)-3-(tetrazol-1-yl)pyrrolidine-1-carbonyl]azetidin-3-yl]oxy-benzonitrile;
[3-[5-(2-Chlorophenoxy)pyrazin-2-yl]azetidin-1-yl]-[(3R)-3-(tetrazol-1-yl)pyrrolidin-1-yl]methanone;
[(3S)-3-(Tetrazol-1-yl)pyrrolidin-1-yl]-[6-[6-(trifluoromethyl)pyrazin-2-yl]oxy-2-azaspiro[3.3]heptan-2-yl]methanone;
[3-[4-[3-(2,2-Dimethylpropyl)triazol-4-yl]phenyl]azetidin-1-yl]-[(3S)-3-(tetrazol-1-yl)pyrrolidin-1-yl]methanone;
[3-[4-(4-Fluorophenoxy)phenyl]azetidin-1-yl]-[(3R)-3-(tetrazol-1-yl)pyrrolidin-1-yl]methanone;
[3-[5-(2,4-Dichlorophenoxy)pyrazin-2-yl]azetidin-1-yl]-[(3R)-3-(tetrazol-1-yl)pyrrolidin-1-yl]methanone;
2-Cyclopropyl-6-[1-[(3S)-3-(tetrazol-1-yl)pyrrolidine-1-carbonyl]azetidin-3-yl]oxy-benzonitrile;
Methyl 2,2-dimethyl-3-[3-[1-[(3R)-3-(tetrazol-1-yl)pyrrolidine-1-carbonyl]azetidin-3-yl]oxyphenyl]propanoate;
[3-[6-(2-Chlorophenoxy)-3-pyridyl]azetidin-1-yl]-[(3S)-3-(tetrazol-1-yl)pyrrolidin-1-yl]methanone;
[3-[4-(4-Cyclopropylpyrimidin-2-yl)oxyphenyl]azetidin-1-yl]-[(3S)-3-(tetrazol-1-yl)pyrrolidin-1-yl]methanone;
[3-[5-(2,4-Dichlorophenoxy)pyrazin-2-yl]azetidin-1-yl]-[(3S)-3-(tetrazol-1-yl)pyrrolidin-1-yl]methanone;
1-[4-[1-[(3R)-3-(Tetrazol-1-yl)pyrrolidine-1-carbonyl]azetidin-3-yl]phenyl]cyclopropanecarbonitrile;
[3-[4-(4-Cyclopropylpyrimidin-2-yl)oxyphenyl]azetidin-1-yl]-[(3R)-3-(tetrazol-1-yl)pyrrolidin-1-yl]methanone;
1-[4-[1-[(3S)-3-(Tetrazol-1-yl)pyrrolidine-1-carbonyl]azetidin-3-yl]phenyl]cyclopropanecarbonitrile;
Methyl 2,2-dimethyl-3-[3-[1-[(3S)-3-(tetrazol-1-yl)pyrrolidine-1-carbonyl]azetidin-3-yl]oxyphenyl]propanoate;
[3-[4-[3-(2,2-Dimethylpropyl)triazol-4-yl]phenyl]azetidin-1-yl]-[(3R)-3-(tetrazol-1-yl)pyrrolidin-1-yl]methanone;
[3-[4-(4-Chloro-2-methylsulfonyl-phenyl)phenyl]azetidin-1-yl]-[(3R)-3-hydroxypyrrolidin-1-yl]methanone;
[3-[4-(4-Chloro-2-methylsulfonyl-phenyl)phenyl]azetidin-1-yl]-[(3R)-3-(hydroxymethyl)pyrrolidin-1-yl]methanone;
[3-[4-(4-Chloro-2-methylsulfonyl-phenyl)phenyl]azetidin-1-yl]-[(3S)-3-(hydroxymethyl)pyrrolidin-1-yl]methanone;
(3S)-1-[6-[5-(Trifluoromethyl)pyrazin-2-yl]methyl]-2-azaspiro[3.3]heptane-2-carbonyl]pyrrolidine-3-carboxamide;
[7-[2-Fluoro-4-(trifluoromethyl)phenyl]methyl]-2,7-diazaspiro[3.5]nonan-2-yl]-[3-(4H-1,2,4-triazol-3-yl)pyrrolidin-1-yl]methanone;
[7-[2-Fluoro-4-(trifluoromethyl) phenoxy]-2-azaspiro[3.5]nonan-2-yl]-[3-(4H-1,2,4-triazol-3-yl)pyrrolidin-1-yl]methanone;
[6-[2-Fluoro-4-(trifluoromethyl)anilino]-2-azaspiro[3.3]heptan-2-yl]-[3-(4H-1,2,4-triazol-3-yl)pyrrolidin-1-yl]methanone;
[6-[[2-Fluoro-4-(trifluoromethyl)phenyl]methylamino]-2-azaspiro[3.3]heptan-2-yl]-[3-(4H-1,2,4-triazol-3-yl)pyrrolidin-1-yl]methanone;
(3S)-1-[3-[4-[3-(Methylsulfonylmethyl)azetidin-1-yl]phenyl]azetidine-1-carbonyl]pyrrolidine-3-carboxamide;
rac-(3S)-1-[3-[5-[3-(Trifluoromethyl)pyrrolidin-1-yl]pyrazin-2-yl]azetidine-1-carbonyl]pyrrolidine-3-carboxamide;
[(3S)-3-(1,2,4-Triazol-4-yl)pyrrolidin-1-yl]-[3-[4-[3-(trifluoromethyl)azetidin-1-yl]phenyl]azetidin-1-yl]methanone;
[6-[[2-Fluoro-4-(trifluoromethyl)phenyl]methylamino]-2-azaspiro[3.3]heptan-2-yl]-[(3S)-3-(1,2,4-triazol-4-yl)pyrrolidin-1-yl]methanone;
[6-[(4,4-Difluoro-1-piperidyl)methyl]-2-azaspiro[3.3]heptan-2-yl]-[3-(4H-1,2,4-triazol-3-yl)pyrrolidin-1-yl]methanone;
[3-[(4,4-Difluorocyclohexyl)methoxy]azetidin-1-yl]-[3-(4H-1,2,4-triazol-3-yl)pyrrolidin-1-yl]methanone;
N-[2-[(3S)-3-(1H-1,2,4-Triazol-5-yl)pyrrolidine-1-carbonyl]-2-azaspiro[3.3]heptan-6-yl]-1-(trifluoromethyl)cyclopropanecarboxamide;
[(3S)-3-(1H-1,2,4-Triazol-5-yl)pyrrolidin-1-yl]-[3-[4-[[1-(trifluoromethyl)cyclopropyl]amino]phenyl]azetidin-1-yl]methanone;
1-[4-[1-[(3S)-3-(1H-1,2,4-Triazol-5-yl)pyrrolidine-1-carbonyl]azetidin-3-yl]phenyl]cyclopropanecarbonitrile;
[(3S)-3-(1H-1,2,4-Triazol-5-yl)pyrrolidin-1-yl]-[3-[6-[1-(trifluoromethyl)cyclopropyl]-3-pyridyl]azetidin-1-yl]methanone;
[(3S)-3-(1H-1,2,4-Triazol-5-yl)pyrrolidin-1-yl]-[3-[2-[1-(trifluoromethyl)cyclopropyl]pyrimidin-5-yl]azetidin-1-yl]methanone;
[3-[(2-Fluoro-4-methylsulfonyl-phenyl)methoxy]azetidin-1-yl]-[(3S)-3-(1H-1,2,4-triazol-5-yl)pyrrolidin-1-yl]methanone;
[(3S)-3-(1H-1,2,4-Triazol-5-yl)pyrrolidin-1-yl]-[6-[6-(trifluoromethyl)-3-pyridyl]oxy]-2-azaspiro[3.3]heptan-2-yl]methanone;
[(3S)-3-(1H-1,2,4-Triazol-5-yl)pyrrolidin-1-yl]-[6-[1-(trifluoromethyl)cyclopropyl]methoxy]-2-azaspiro[3.3]heptan-2-yl]methanone;
[(3S)-3-(1H-1,2,4-Triazol-5-yl)pyrrolidin-1-yl]-[2-[2-(trifluoromethyl)phenyl]sulfonyl-2,6-diazaspiro[3.3]heptan-6-yl]methanone;
[(3S)-3-(1H-1,2,4-Triazol-5-yl)pyrrolidin-1-yl]-[2-[3-(trifluoromethoxy)phenyl]sulfonyl-2,6-diazaspiro[3.3]heptan-6-yl]methanone;
N-Methyl-2-[[2-[(3S)-3-(1H-1,2,4-triazol-5-yl)pyrrolidine-1-carbonyl]-2,6-diazaspiro[3.3]heptan-6-yl]methyl]benzamide;
[(3S)-3-(1H-1,2,4-Triazol-5-yl)pyrrolidin-1-yl]-[2-[3-(trifluoromethyl)phenyl]sulfonyl-2,6-diazaspiro[3.3]heptan-6-yl]methanone;
[(3S)-3-(1,2,4-Triazol-4-yl)pyrrolidin-1-yl]-[3-[2-[1-(trifluoromethyl)cyclopropyl]pyrimidin-5-yl]azetidin-1-yl]methanone;
N-[1-[(3S)-3-(1,2,4-Triazol-4-yl)pyrrolidine-1-carbonyl]azetidin-3-yl]methyl]-3-(trifluoromethyl)benzenesulfonamide;
[3-[4-(3-Dimethylphosphorylphenyl)phenyl]azetidin-1-yl]-[(3S)-3-(1,2,4-triazol-4-yl)pyrrolidin-1-yl]methanone;
[(3S)-3-(1H-Triazol-5-yl)pyrrolidin-1-yl]-[3-[6-(trifluoromethyl)pyridazin-3-yl]oxyazetidin-1-yl]methanone;
1-[3-[4-(4-Chloro-2-methylsulfonyl-phenyl)phenyl]azetidine-1-carbonyl]pyrrolidine-3-sulfonamide;
tert-Butyl N-[(3S)-1-[3-[4-(4-chloro-2-methylsulfonyl-phenyl)phenyl]azetidine-1-carbonyl]pyrrolidin-3-yl]carbamate;
(3S)-1-[3-[5-(2-Chloro-4-methylsulfonyl-phenyl)-2-pyridyl]azetidine-1-carbonyl]pyrrolidine-3-carboxamide;
N-[1-[(3S)-3-(1H-1,2,4-Triazol-5-yl)pyrrolidine-1-carbonyl]azetidin-3-yl]methyl]-3-(trifluoromethyl)benzenesulfonamide;
N-[1-[(3S)-3-(1H-1,2,4-Triazol-5-yl)pyrrolidine-1-carbonyl]azetidin-3-yl]methyl]-4-(trifluoromethyl)benzenesulfonamide;
[(3S)-3-(1H-1,2,4-Triazol-5-yl)pyrrolidin-1-yl]-[2-[4-(trifluoromethoxy)phenyl]sulfonyl-2,6-diazaspiro[3.3]heptan-6-yl]methanone;
[3-[5-(2,4-Difluorophenyl)-4H-1,2,4-triazol-3-yl]azetidin-1-yl]-[(3S)-3-(1H-1,2,4-triazol-5-yl)pyrrolidin-1-yl]methanone;
(3S)-1-[3-[5-(4-Chloro-2-methylsulfonyl-phenyl)-2-pyridyl]azetidine-1-carbonyl]pyrrolidine-3-carboxamide;
(3S)-1-[3-[5-(2-Chloro-4-methylsulfonyl-phenyl)pyrazin-2-yl]azetidine-1-carbonyl]pyrrolidine-3-carboxamide;
(3R)-1-[3-[[2-Fluoro-4-(trifluoromethyl)phenyl]methoxy]azetidine-1-carbonyl]pyrrolidine-3-carboxamide;
(3R)-1-[6-[[6-(Trifluoromethyl)-3-pyridyl]oxy]-2-azaspiro[3.3]heptane-2-carbonyl]pyrrolidine-3-carboxamide;
(3S)-1-[3-[[2-Fluoro-4-(trifluoromethyl)phenyl]methylamino]azetidine-1-carbonyl]pyrrolidine-3-carboxamide;
[3-[[2-Fluoro-4-(trifluoromethyl)phenyl]methylamino]azetidin-1-yl]-[(3S)-3-(1H-1,2,4-triazol-5-yl)pyrrolidin-1-yl]methanone;
[3-[[2-Fluoro-4-(trifluoromethyl)phenyl]methylamino]azetidin-1-yl]-[(3R)-3-(1H-1,2,4-triazol-5-yl)pyrrolidin-1-yl]methanone;
2-[(3S)-3-(1H-1,2,4-Triazol-5-yl)pyrrolidine-1-carbonyl]-N-[[1-(trifluoromethyl)cyclopropyl]methyl]-2-azaspiro[3.3]heptane-6-carboxamide;
[(3S)-3-(1,2,4-Triazol-4-yl)pyrrolidin-1-yl]-[6-[[[1-(trifluoromethyl)cyclopropyl]amino]methyl]-2-azaspiro[3.3]heptan-2-yl]methanone;
[3-[[2-Fluoro-4-(trifluoromethyl)phenyl]methylamino]azetidin-1-yl]-[(3R)-3-(tetrazol-1-yl)pyrrolidin-1-yl]methanone;
[3-[4-(Benzenesulfonyl)phenyl]azetidin-1-yl]-[(3S)-3-(tetrazol-1-yl)pyrrolidin-1-yl]methanone;
[(3S)-3-(Tetrazol-1-yl)pyrrolidin-1-yl]-[3-[4-[4-(trifluoromethyl)pyrimidin-2-yl]oxyphenyl]azetidin-1-yl]methanone;
[3-[6-(2-Chlorophenoxy)pyridazin-3-yl]azetidin-1-yl]-[(3S)-3-(tetrazol-1-yl)pyrrolidin-1-yl]methanone;
[3-[[2-Fluoro-4-(trifluoromethyl)phenyl]methylamino]azetidin-1-yl]-[(3S)-3-(tetrazol-1-yl)pyrrolidin-1-yl]methanone;
Methyl 2-methyl-2-[3-[1-[(3S)-3-(tetrazol-1-yl)pyrrolidine-1-carbonyl]azetidin-3-yl]oxyphenyl]propanoate;
[2-[4-Fluoro-2-(trifluoromethyl)phenyl]sulfonyl-2,6-diazaspiro[3.3]heptan-6-yl]-[(3S)-3-(tetrazol-1-yl)pyrrolidin-1-yl]methanone;
Methyl 2-methyl-2-[3-[1-[(3R)-3-(tetrazol-1-yl)pyrrolidine-1-carbonyl]azetidin-3-yl]oxyphenyl]propanoate;
[(3R)-3-(Tetrazol-1-yl)pyrrolidin-1-yl]-[3-[4-[4-(trifluoromethyl)pyrimidin-2-yl]oxyphenyl]azetidin-1-yl]methanone;
[2-[4-Fluoro-3-(trifluoromethyl)phenyl]sulfonyl-2,6-diazaspiro[3.3]heptan-6-yl]-[(3S)-3-(tetrazol-1-yl)pyrrolidin-1-yl]methanone;
[3-[(2-Fluoro-4-methylsulfonyl-phenyl)methoxy]azetidin-1-yl]-[(3S)-3-(tetrazol-1-yl)pyrrolidin-1-yl]methanone;
[2-[4-Fluoro-2-(trifluoromethyl)phenyl]sulfonyl-2,6-diazaspiro[3.3]heptan-6-yl]-[(3R)-3-(tetrazol-1-yl)pyrrolidin-1-yl]methanone;
[3-[4-(4-Chloro-2-methylsulfonyl-phenyl)phenyl]azetidin-1-yl]-[(3S)-3-(tetrazol-1-yl)pyrrolidin-1-yl]methanone;
[3-[4-(Dimethylphosphorylmethyl)phenyl]azetidin-1-yl]-[(3S)-3-(1H-triazol-5-yl)pyrrolidin-1-yl]methanone;
[7-[2-Fluoro-4-(trifluoromethyl) phenoxy]-2-azaspiro[3.5]nonan-2-yl]-[(3S)-3-(1,2,4-triazol-4-yl)pyrrolidin-1-yl]methanone;
(3S)-1-[3-[4-(2-Chloro-4-methylsulfonyl-phenyl)phenyl]azetidine-1-carbonyl]pyrrolidine-3-carboxamide;
[3-[4-(2-Chloro-4-methylsulfonyl-phenyl)phenyl]azetidin-1-yl]-[(3S)-3-hydroxypyrrolidin-1-yl]methanone;
[3-[4-(4-Chloro-2-methylsulfonyl-phenyl)phenyl]azetidin-1-yl]-[(3S)-3-hydroxypyrrolidin-1-yl]methanone;
(3S)-1-[3-[4-(6-Chloro-4-methylsulfonyl-3-pyridyl)phenyl]azetidine-1-carbonyl]pyrrolidine-3-carboxamide;
[3-[4-(4-Chloro-2-methylsulfonyl-phenyl)phenyl]azetidin-1-yl]-[(3S)-3-(1H-1,2,4-triazol-5-yl)pyrrolidin-1-yl]methanone;
(3-Pyrrolidin-1-ylsulfonylpyrrolidin-1-yl)-[3-[4-[1-(trifluoromethyl)cyclopropyl]phenyl]azetidin-1-yl]methanone;
Morpholino-[3-[4-[1-(trifluoromethyl)cyclopropyl]phenyl]azetidin-1-yl]methanone;
[3-[5-(4-Chloro-2-fluoro-phenoxy)pyrazin-2-yl]azetidin-1-yl]-morpholino-methanone;
[3-[[rac-(1S,5R)-6,6-difluoro-3-bicyclo[3.1.0]hexanyl]methoxy]azetidin-1-yl]-[3-(4H-1,2,4-triazol-3-yl)pyrrolidin-1-yl]methanone;
[6-[[2-Fluoro-4-(trifluoromethyl)phenyl]methylamino]-2-azaspiro[3.3]heptan-2-yl]-[(3S)-3-(4H-1,2,4-triazol-3-yl)pyrrolidin-1-yl]methanone;
[7-[[2-Fluoro-4-(trifluoromethyl)phenyl]methyl]-2,7-diazaspiro[3.5]nonan-2-yl]-[(3S)-3-(4H-1,2,4-triazol-3-yl)pyrrolidin-1-yl]methanone;
[7-[[2-Fluoro-4-(trifluoromethyl)phenyl]methyl]-2,7-diazaspiro[3.5]nonan-2-yl]-[(3R)-3-(4H-1,2,4-triazol-3-yl)pyrrolidin-1-yl]methanone;
[7-[2-Fluoro-4-(trifluoromethyl) phenoxy]-2-azaspiro[3.5]nonan-2-yl]-[(3S)-3-(4H-1,2,4-triazol-3-yl)pyrrolidin-1-yl]methanone;
[7-[2-Fluoro-4-(trifluoromethyl) phenoxy]-2-azaspiro[3.5]nonan-2-yl]-[(3R)-3-(4H-1,2,4-triazol-3-yl)pyrrolidin-1-yl]methanone;
(3S)-1-[3-[5-(4-Chloro-2-fluoro-phenyl)pyrimidin-2-yl]azetidine-1-carbonyl]pyrrolidine-3-carboxamide;
(3S)-1-[3-[5-(4-Chloro-2-fluoro-phenyl)-2-pyridyl]azetidine-1-carbonyl]pyrrolidine-3-carboxamide;
(3S)-1-[3-[5-(2,4-Dichlorophenyl)-2-pyridyl]azetidine-1-carbonyl]pyrrolidine-3-carboxamide;
(3S)-1-[3-[4-(5-Chloro-3-methylsulfonyl-2-pyridyl)phenyl]azetidine-1-carbonyl]pyrrolidine-3-carboxamide;
(3S)-1-[3-[6-(2-Chloro-4-methylsulfonyl-phenyl)-3-pyridyl]azetidine-1-carbonyl]pyrrolidine-3-carboxamide;
(3S)-1-[3-[6-(4-Chloro-2-methylsulfonyl-phenyl)-3-pyridyl]azetidine-1-carbonyl]pyrrolidine-3-carboxamide;
[3-[4-(6-Chloro-4-methylsulfonyl-3-pyridyl)phenyl]azetidin-1-yl]-[(3S)-3-hydroxypyrrolidin-1-yl]methanone;
(3S)-1-[3-[4-[5-(Trifluoromethyl)pyrazin-2-yl]phenyl]azetidine-1-carbonyl]pyrrolidine-3-carboxamide;
[(3S)-3-Aminopyrrolidin-1-yl]-[3-[4-(4-chloro-2-methylsulfonyl-phenyl)phenyl]azetidin-1-yl]methanone;
[2-[[2-Fluoro-4-(trifluoromethyl)phenyl]methyl]-2,7-diazaspiro[3.5]nonan-7-yl]-[(3S)-3-(4H-1,2,4-triazol-3-yl)pyrrolidin-1-yl]methanone;
[2-[[2-Fluoro-4-(trifluoromethyl)phenyl]methyl]-2,7-diazaspiro[3.5]nonan-7-yl]-[(3R)-3-(4H-1,2,4-triazol-3-yl)pyrrolidin-1-yl]methanone;
(3S)-1-[7-[[2-Fluoro-4-(trifluoromethyl)phenyl]methyl]-2,7-diazaspiro[3.5]nonane-2-carbonyl]pyrrolidine-3-carboxamide;
(3R)-1-[7-[[2-Fluoro-4-(trifluoromethyl)phenyl]methyl]-2,7-diazaspiro[3.5]nonane-2-carbonyl]pyrrolidine-3-carboxamide;
(3S)-1-[7-[2-Fluoro-4-(trifluoromethyl) phenoxy]-2-azaspiro[3.5]nonane-2-carbonyl]pyrrolidine-3-carboxamide;
(3R)-1-[7-[2-Fluoro-4-(trifluoromethyl) phenoxy]-2-azaspiro[3.5]nonane-2-carbonyl]pyrrolidine-3-carboxamide;
(3S)-1-[2-[2-Fluoro-4-(trifluoromethyl)phenyl]methyl]-2,7-diazaspiro[3.5]nonane-7-carbonyl]pyrrolidine-3-carboxamide;
(3R)-1-[2-[2-Fluoro-4-(trifluoromethyl)phenyl]methyl]-2,7-diazaspiro[3.5]nonane-7-carbonyl]pyrrolidine-3-carboxamide;
[7-[2-Fluoro-4-(trifluoromethyl)phenyl]sulfonyl-2,7-diazaspiro[3.5]nonan-2-yl]-[(3S)-3-(1H-1,2,4-trizol-5-yl)pyrrolidin-1-yl]methanon;
(3S)-1-[7-[2-Fluoro-4-(trifluoromethyl)phenyl]sulfonyl-2,7-diazaspiro[3.5]nonane-2-carbonyl]pyrrolidine-3-carboxamide;
[6-[(3,5-Difluoro-2-pyridyl)methyl]-2-azaspiro[3.3]heptan-2-yl]-[(3S)-3-(tetrazol-1-yl)pyrrolidin-1-yl]methanone;
[(3S)-3-(Tetrazol-1-yl)pyrrolidin-1-yl]-[6-[6-(trifluoromethyl)-3-pyridyl]methyl]-2-azaspiro[3.3]heptan-2-yl]methanone;
[6-[(5-Fluoro-3-pyridyl)methyl]-2-azaspiro[3.3]heptan-2-yl]-[(3S)-3-(tetrazol-1-yl)pyrrolidin-1-yl]methanone;
[6-[(3,5-Difluoro-2-pyridyl)methyl]-2-azaspiro[3.3]heptan-2-yl]-[(3R)-3-(tetrazol-1-yl)pyrrolidin-1-yl]methanone;
[(3R)-3-(Tetrazol-1-yl)pyrrolidin-1-yl]-[6-[6-(trifluoromethyl)-3-pyridyl]methyl]-2-azaspiro[3.3]heptan-2-yl]methanone;
[6-[(5-Fluoro-3-pyridyl)methyl]-2-azaspiro[3.3]heptan-2-yl]-[(3R)-3-(tetrazol-1-yl)pyrrolidin-1-yl]methanone;
[3-(4H-1,2,4-Triazol-3-yl)pyrrolidin-1-yl]-[7-[2-(trifluoromethyl)pyrimidin-4-yl]oxy-2-azaspiro[3.5]nonan-2-yl]methanone;
N-[2-[3-(4H-1,2,4-Triazol-3-yl)pyrrolidine-1-carbonyl]-2-azaspiro[3.5]nonan-7-yl]-3-(trifluoromethoxy)benzenesulfonamide;
(3S)-1-[3-[4-(4-Chloro-2-fluoro-phenyl)-3-methylsulfonyl-phenyl]azetidine-1-carbonyl]pyrrolidine-3-carboxamide;
N-[2-[(3S)-3-(1H-1,2,4-Triazol-5-yl)pyrrolidine-1-carbonyl]-2-azaspiro[3.3]heptan-6-yl]-2-(trifluoromethyl)benzenesulfonamide;
N-[2-[(3S)-3-(1H-1,2,4-Triazol-5-yl)pyrrolidine-1-carbonyl]-2-azaspiro[3.3]heptan-6-yl]-3-(trifluoromethyl)benzenesulfonamide;
N-[2-[(3S)-3-(1H-1,2,4-Triazol-5-yl)pyrrolidine-1-carbonyl]-2-azaspiro[3.3]heptan-6-yl]-4-(trifluoromethyl)benzenesulfonamide;
[(3S)-3-(1H-1,2,4-Triazol-5-yl)pyrrolidin-1-yl]-[6-[5-(trifluoromethyl)-2-pyridyl]methyl]-2-azaspiro[3.3]heptan-2-yl]methanone;
[(3S)-3-(1H-1,2,4-Triazol-5-yl)pyrrolidin-1-yl]-[6-[6-(trifluoromethyl)-3-pyridyl]methyl]-2-azaspiro[3.3]heptan-2-yl]methanone;
[(3S)-3-(1H-1,2,4-Triazol-5-yl)pyrrolidin-1-yl]-[6-[5-(trifluoromethyl)pyrazin-2-yl]methyl]-2-azaspiro[3.3]heptan-2-yl]methanone;
(3S)-1-[6-[2-(Trifluoromethyl)phenyl]sulfonylamino]-2-azaspiro[3.3]heptane-2-carbonyl]pyrrolidine-3-carboxamide;
(3S)-1-[6-[3-(Trifluoromethyl)phenyl]sulfonylamino]-2-azaspiro[3.3]heptane-2-carbonyl]pyrrolidine-3-carboxamide;
(3S)-1-[6-[4-(Trifluoromethyl)phenyl]sulfonylamino]-2-azaspiro[3.3]heptane-2-carbonyl]pyrrolidine-3-carboxamide;
(3S)-1-[6-(2,2-Dimethylpropylsulfonylamino)-2-azaspiro[3.3]heptane-2-carbonyl]pyrrolidine-3-carboxamide;
(3S)-1-[3-[5-(2-Chloro-4-methylsulfonyl-phenyl)pyrimidin-2-yl]azetidine-1-carbonyl]pyrrolidine-3-carboxamide;
(3S)-1-[3-[2-(2-Chloro-4-methylsulfonyl-phenyl)pyrimidin-5-yl]azetidine-1-carbonyl]pyrrolidine-3-carboxamide;
[3-[6-(1,1-Dioxo-1,4-thiazinan-4-yl)-3-pyridyl]azetidin-1-yl]-[3-(1H-1,2,4-triazol-5-yl)pyrrolidin-1-yl]methanone;
[3-[5-(4-Chloro-2-methylsulfonyl-phenyl)-2-pyridyl]azetidin-1-yl]-[(3R)-3-hydroxypyrrolidin-1-yl]methanone;
[3-(4H-1,2,4-Triazol-3-yl)pyrrolidin-1-yl]-[6-[6-(trifluoromethyl)-3-pyridyl]methyl]-2-azaspiro[3.3]heptan-2-yl]methanone;
1-[6-[[6-(Trifluoromethyl)-3-pyridyl]methyl]-2-azaspiro[3.3]heptane-2-carbonyl]pyrrolidine-3-sulfonamide;
1-[6-[6-(Trifluoromethyl)-3-pyridyl]methyl]-2-azaspiro[3.3]heptane-2-carbonyl]piperidine-3-carboxamide;
[3-[5-(4-Chlorophenoxy)pyrazin-2-yl]azetidin-1-yl]-[(3S)-3-(tetrazol-1-yl)pyrrolidin-1-yl]methanone;
[(3S)-3-(Tetrazol-1-yl)pyrrolidin-1-yl]-[6-[4-(trifluoromethyl)phenyl]-2-azaspiro[3.3]heptan-2-yl]methanone;
[(3S)-3-(Tetrazol-1-yl)pyrrolidin-1-yl]-[6-[2-(trifluoromethyl)pyrimidin-5-yl]-2-azaspiro[3.3]heptan-2-yl]methanone;
[3-(4H-1,2,4-Triazol-3-yl)pyrrolidin-1-yl]-[7-[6-(trifluoromethyl)pyrazin-2-yl]oxy-2-azaspiro[3.5]nonan-2-yl]methanone;
[7-(4-Fluoro-2-methylsulfonyl-phenoxy)-2-azaspiro[3.5]nonan-2-yl]-[3-(4H-1,2,4-triazol-3-yl)pyrrolidin-1-yl]methanone;
[(3S)-3-(1,2,4-Triazol-4-yl)pyrrolidin-1-yl]-[3-[6-[1-(trifluoromethyl)cyclopropyl]methylamino]-3-pyridyl]azetidin-1-yl]methanone;
[3-(4H-1,2,4-Triazol-3-yl)pyrrolidin-1-yl]-[7-[6-(trifluoromethyl)pyridazin-3-yl]oxy-2-azaspiro[3.5]nonan-2-yl]methanone;
[3-(4H-1,2,4-Triazol-3-yl)pyrrolidin-1-yl]-[7-[5-(trifluoromethyl)pyrazin-2-yl]oxy-2-azaspiro[3.5]nonan-2-yl]methanone;
[(3S)-3-(1,2,4-Triazol-4-yl)pyrrolidin-1-yl]-[7-[5-(trifluoromethyl)pyrazin-2-yl]oxy-2-azaspiro[3.5]nonan-2-yl]methanone;
(3R)-1-[3-[4-(4-Chloro-2-methylsulfonyl-phenyl)phenyl]azetidine-1-carbonyl]pyrrolidine-3-sulfonamide;
(3S)-1-[3-[4-(4-Chloro-2-methylsulfonyl-phenyl)phenyl]azetidine-1-carbonyl]pyrrolidine-3-sulfonamide;
3-[3-[4-(4-Chloro-2-methylsulfonyl-phenyl)phenyl]azetidine-1-carbonyl]-4,5-dihydroisoxazole-5-carboxamide;
1-[7-[[2-Fluoro-4-(trifluoromethyl)phenyl]methyl]-2,7-diazaspiro[3.5]nonane-2-carbonyl]piperidine-3-carboxamide;
1-[7-[[2-Fluoro-4-(trifluoromethyl)phenyl]methyl]-2,7-diazaspiro[3.5]nonane-2-carbonyl]pyrrolidine-3-sulfonamide;
[6-[(3-Methylsulfonylphenyl)methyl]-2-azaspiro[3.3]heptan-2-yl]-[(3S)-3-(1H-1,2,4-triazol-5-yl)pyrrolidin-1-yl]methanone;
[6-[(4-Methylsulfonylphenyl)methyl]-2-azaspiro[3.3]heptan-2-yl]-[(3S)-3-(1H-1,2,4-triazol-5-yl)pyrrolidin-1-yl]methanone;
(3S)-1-[6-[(3-Methylsulfonylphenyl)methyl]-2-azaspiro[3.3]heptane-2-carbonyl]pyrrolidine-3-carboxamide;
(3S)-1-[6-[(4-Methylsulfonylphenyl)methyl]-2-azaspiro[3.3]heptane-2-carbonyl]pyrrolidine-3-carboxamide;
[(3S)-3-(1H-1,2,4-Triazol-5-yl)pyrrolidin-1-yl]-[3-[6-[[1-(trifluoromethyl)cyclopropyl]methylamino]-3-pyridyl]azetidin-1-yl]methanone;
(3S)-1-[3-[6-[1-(Trifluoromethyl)cyclopropyl]methylamino]-3-pyridyl]azetidine-1-carbonyl]pyrrolidine-3-carboxamide;
4-Fluoro-N-[1-[(3S)-3-(1H-1,2,4-triazol-5-yl)pyrrolidine-1-carbonyl]-4-piperidyl]benzenesulfonamide;
N-[1-[(3S)-3-(1H-1,2,4-Triazol-5-yl)pyrrolidine-1-carbonyl]-4-piperidyl]-4-(trifluoromethyl)benzenesulfonamide;
(3S)-1-[4-[4-(Trifluoromethyl)phenyl]sulfonylamino]piperidine-1-carbonyl]pyrrolidine-3-carboxamide;
4-Chloro-N-[1-[(3S)-3-(1H-1,2,4-triazol-5-yl)pyrrolidine-1-carbonyl]-4-piperidyl]methyl]benzenesulfonamide;
(3S)-1-[7-(4-Fluoro-2-methylsulfonyl-phenoxy)-2-azaspiro[3.5]nonane-2-carbonyl]pyrrolidine-3-carboxamide;
(3S)-1-[7-[6-(Trifluoromethyl)pyridazin-3-yl]oxy-2-azaspiro[3.5]nonane-2-carbonyl]pyrrolidine-3-carboxamide;
(3S)-1-[7-[2-(Trifluoromethyl)pyrimidin-4-yl]oxy-2-azaspiro[3.5]nonane-2-carbonyl]pyrrolidine-3-carboxamide;
(3S)-1-[7-[3-(Trifluoromethoxy)phenyl]sulfonylamino]-2-azaspiro[3.5]nonane-2-carbonyl]pyrrolidine-3-carboxamide;
N-[2-[(3S)-3-(1H-1,2,4-Triazol-5-yl)pyrrolidine-1-carbonyl]-2-azaspiro[3.5]nonan-7-yl]-3-(trifluoromethoxy)benzenesulfonamide;
(5S)-3-[3-[4-(4-Chloro-2-methylsulfonyl-phenyl)phenyl]azetidine-1-carbonyl]-4,5-dihydroisoxazole-5-carboxamide;
(5R)-3-[3-[4-(4-Chloro-2-methylsulfonyl-phenyl)phenyl]azetidine-1-carbonyl]-4,5-dihydroisoxazole-5-carboxamide;
1-[3-[4-(4-Chloro-2-methylsulfonyl-phenyl)phenyl]azetidine-1-carbonyl]azetidine-3-sulfonamide;
1-[3-[4-(4-Chloro-2-methylsulfonyl-phenyl)phenyl]azetidine-1-carbonyl]piperidine-4-sulfonamide;
N-[[1-[(3S)-3-(1H-1,2,4-Triazol-5-yl)pyrrolidine-1-carbonyl]-4-piperidyl]methyl]-4-(trifluoromethyl)benzenesulfonamide;
(3S)-1-[4-[[[4-(Trifluoromethyl)phenyl]sulfonylamino]methyl]piperidine-1-carbonyl]pyrrolidine-3-carboxamide;
N-[1-[(3S)-3-(1H-1,2,4-Triazol-5-yl)pyrrolidine-1-carbonyl]-4-piperidyl]methyl]-4-(trifluoromethoxy)benzenesulfonamide;
(3S)-1-[4-[[[4-(Trifluoromethoxy)phenyl]sulfonylamino]methyl]piperidine-1-carbonyl]pyrrolidine-3-carboxamide;
(3S)-1-[3-[4-(6,6-Difluoro-2-azaspiro[3.3]heptan-2-yl)phenyl]azetidine-1-carbonyl]pyrrolidine-3-carboxamide;
(3S)-1-[3-[4-[3-(Trifluoromethyl)cyclobutyl]phenyl]azetidine-1-carbonyl]pyrrolidine-3-carboxamide;
(3S)-1-[3-[4-(2-Azaspiro[3.4]octan-2-yl)phenyl]azetidine-1-carbonyl]pyrrolidine-3-carboxamide;
[(3S)-3-(1H-1,2,4-Triazol-5-yl)pyrrolidin-1-yl]-[6-[[1-(trifluoromethyl)cyclopropyl]methylamino]-2-azaspiro[3.3]heptan-2-yl]methanone;
[6-[(4-Fluoro-2-methylsulfonyl-phenyl)methyl]-2-azaspiro[3.3]heptan-2-yl]-[(3S)-3-(1H-1,2,4-triazol-5-yl)pyrrolidin-1-yl]methanone;
[3-[6-(3-Hydroxy-3-methyl-azetidin-1-yl)-3-pyridyl]azetidin-1-yl]-[(3S)-3-(1H-1,2,4-triazol-5-yl)pyrrolidin-1-yl]methanone;
[3-[6-[3-Hydroxy-3-(trifluoromethyl)azetidin-1-yl]-3-pyridyl]azetidin-1-yl]-[(3S)-3-(1H-1,2,4-triazol-5-yl)pyrrolidin-1-yl]methanone;
[3-[6-[3-Hydroxy-3-(trifluoromethyl)pyrrolidin-1-yl]-3-pyridyl]azetidin-1-yl]-[rac-(3S)-3-(1H-1,2,4-triazol-5-yl)pyrrolidin-1-yl]methanone;
[3-[4-[1-(1H-Tetrazol-5-yl)cyclopropyl]phenyl]azetidin-1-yl]-[(3S)-3-(1H-1,2,4-triazol-5-yl)pyrrolidin-1-yl]methanone;
[3-[4-[1-(2-tert-Butyltetrazol-5-yl)cyclopropyl]phenyl]azetidin-1-yl]-[(3S)-3-(1H-1,2,4-triazol-5-yl)pyrrolidin-1-yl]methanone;
(3S)-1-[6-[1-(Trifluoromethyl)cyclopropyl]methylamino]-2-azaspiro[3.3]heptane-2-carbonyl]pyrrolidine-3-carboxamide;
(3S)-1-[6-[(4-Fluoro-2-methylsulfonyl-phenyl)methyl]-2-azaspiro[3.3]heptane-2-carbonyl]pyrrolidine-3-carboxamide;
(3S)-1-[3-[6-[3-Hydroxy-3-(trifluoromethyl)azetidin-1-yl]-3-pyridyl]azetidine-1-carbonyl]pyrrolidine-3-carboxamide;
rac-(3S)-1-[3-[6-[3-Hydroxy-3-(trifluoromethyl)pyrrolidin-1-yl]-3-pyridyl]azetidine-1-carbonyl]pyrrolidine-3-carboxamide;
(3S)-1-[3-[4-[1-(2-tert-Butyltetrazol-5-yl)cyclopropyl]phenyl]azetidine-1-carbonyl]pyrrolidine-3-carboxamide;
(3S)-1-[6-[[1-(Trifluoromethyl)cyclopropyl]amino]-2-azaspiro[3.3]heptane-2-carbonyl]pyrrolidine-3-carboxamide;
[(3S)-3-(1H-1,2,4-Triazol-5-yl)pyrrolidin-1-yl]-[6-[1-(trifluoromethyl)cyclopropyl]amino]-2-azaspiro[3.3]heptan-2-yl]methanone;
(3S)-1-[3-[6-[(3R)-3-Hydroxy-3-(trifluoromethyl)pyrrolidin-1-yl]-3-pyridyl]azetidine-1-carbonyl]pyrrolidine-3-carboxamide;
(3S)-1-[3-[6-[(3S)-3-Hydroxy-3-(trifluoromethyl)pyrrolidin-1-yl]-3-pyridyl]azetidine-1-carbonyl]pyrrolidine-3-carboxamide;
[3-[4-(4-Fluorophenoxy)phenyl]azetidin-1-yl]-[(3R)-3-(tetrazol-2-yl)pyrrolidin-1-yl]methanone;
[(3R)-3-(Tetrazol-2-yl)pyrrolidin-1-yl]-[3-[4-[1-(trifluoromethyl)cyclopropyl]phenyl]azetidin-1-yl]methanone;
[3-[6-[(3R)-3-Hydroxy-3-(trifluoromethyl)pyrrolidin-1-yl]-3-pyridyl]azetidin-1-yl]-[(3S)-3-(1H-1,2,4-triazol-5-yl)pyrrolidin-1-yl]methanone;
[3-[6-[(3S)-3-Hydroxy-3-(trifluoromethyl)pyrrolidin-1-yl]-3-pyridyl]azetidin-1-yl]-[(3S)-3-(1H-1,2,4-triazol-5-yl)pyrrolidin-1-yl]methanone;
[3-[6-(4-Chloro-2-methylsulfonyl-phenoxy)-3-pyridyl]azetidin-1-yl]-[(3S)-3-(1,2,4-triazol-4-yl)pyrrolidin-1-yl]methanone;
1-[3-[4-[1-(Trifluoromethyl)cyclopropyl]phenyl]azetidine-1-carbonyl]pyrrolidine-3-sulfonamide;
1-[3-[[2-Fluoro-4-(trifluoromethyl)phenyl]methoxy]azetidine-1-carbonyl]pyrrolidine-3-sulfonamide;
[(3S)-3-(1H-1,2,4-Triazol-5-yl)pyrrolidin-1-yl]-[3-[[4-(trifluoromethylsulfonyl)phenyl]methoxy]azetidin-1-yl]methanone;
[(3S)-3-(1H-1,2,4-Triazol-5-yl)pyrrolidin-1-yl]-[3-[[3-(trifluoromethylsulfonyl)phenyl]methoxy]azetidin-1-yl]methanone;
Methyl 2-[4-[1-[(3S)-3-(1H-1,2,4-triazol-5-yl)pyrrolidine-1-carbonyl]azetidin-3-yl]phenyl]benzoate;
1-[4-[1-[rac-(3S)-3-(1H-1,2,4-Triazol-5-yl)pyrrolidine-1-carbonyl]azetidin-3-yl]phenyl]piperidine-2-carboxamide;
[3-[[2-Fluoro-5-(trifluoromethyl)phenyl]methylamino]azetidin-1-yl]-[(3S)-3-(1H-1,2,4-triazol-5-yl)pyrrolidin-1-yl]methanone;
[(3S)-3-(1H-1,2,4-Triazol-5-yl)pyrrolidin-1-yl]-[3-[[4-(trifluoromethyl)phenyl]methylamino]azetidin-1-yl]methanone;
[(3S)-3-(Tetrazol-1-yl)pyrrolidin-1-yl]-[3-[2-[1-(trifluoromethyl)cyclopropyl]pyrimidin-5-yl]azetidin-1-yl]methanone;
(3S)-1-[7-[5-(Trifluoromethyl)pyrazin-2-yl]amino]-2-azaspiro[3.5]nonane-2-carbonyl]pyrrolidine-3-carboxamide;
(3R)-1-[7-[5-(Trifluoromethyl)pyrazin-2-yl]amino]-2-azaspiro[3.5]nonane-2-carbonyl]pyrrolidine-3-carboxamide;
(3S)-1-[7-[5-(Trifluoromethyl)pyrimidin-2-yl]oxy-2-azaspiro[3.5]nonane-2-carbonyl]pyrrolidine-3-carboxamide;
[3-[4-(4-Fluorophenoxy)phenyl]azetidin-1-yl]-[(3S)-3-(tetrazol-2-yl)pyrrolidin-1-yl]methanone;
[3-[4-(4-Chloro-2-methylsulfonyl-phenyl)phenyl]azetidin-1-yl]-[(3S)-3-(tetrazol-2-yl)pyrrolidin-1-yl]methanone;
[3-[4-(4-Chloro-2-methylsulfonyl-phenyl)phenyl]azetidin-1-yl]-[(3R)-3-(tetrazol-2-yl)pyrrolidin-1-yl]methanone;
[(3S)-3-(Tetrazol-1-yl)pyrrolidin-1-yl]-[3-[4-[1-(trifluoromethyl)cyclopropyl]methoxy]phenyl]azetidin-1-yl]methanone;
[(3S)-3-(Tetrazol-1-yl)pyrrolidin-1-yl]-[3-[4-[5-(trifluoromethyl)pyrazin-2-yl]oxyphenyl]azetidin-1-yl]methanone;
[(3S)-3-(1H-1,2,4-Triazol-5-yl)pyrrolidin-1-yl]-[3-[6-[[1-(trifluoromethyl)cyclopropyl]amino]-3-pyridyl]azetidin-1-yl]methanone;
4,4-Difluoro-1-[4-[1-[rac-(3S)-3-(1H-1,2,4-triazol-5-yl)pyrrolidine-1-carbonyl]azetidin-3-yl]phenyl]piperidine-2-carboxamide;
(3S)-1-[7-[5-(Trifluoromethyl)pyrazin-2-yl]oxy-2-azaspiro[3.5]nonane-2-carbonyl]pyrrolidine-3-carboxamide;
[3-[[5-Chloro-2-(trifluoromethyl)phenyl]methylamino]azetidin-1-yl]-[(3S)-3-(1H-1,2,4-triazol-5-yl)pyrrolidin-1-yl]methanone;
[3-[[3-Chloro-5-(trifluoromethyl)phenyl]methylamino]azetidin-1-yl]-[(3S)-3-(1H-1,2,4-triazol-5-yl)pyrrolidin-1-yl]methanone;
[3-[[2-Fluoro-4-(trifluoromethoxy)phenyl]methylamino]azetidin-1-yl]-[(3S)-3-(1H-1,2,4-triazol-5-yl)pyrrolidin-1-yl]methanone;
[3-[[3-Fluoro-4-(trifluoromethyl)phenyl]methylamino]azetidin-1-yl]-[(3S)-3-(1H-1,2,4-triazol-5-yl)pyrrolidin-1-yl]methanone;
[3-[[2-Chloro-4-(trifluoromethyl)phenyl]methylamino]azetidin-1-yl]-[(3S)-3-(1H-1,2,4-triazol-5-yl)pyrrolidin-1-yl]methanone;
2-[4-[1-[(3S)-3-(1H-1,2,4-Triazol-5-yl)pyrrolidine-1-carbonyl]azetidin-3-yl]phenyl]benzoic acid;
(3S)-1-[3-[4-(3,5-Dimethylpyrazol-1-yl)phenyl]azetidine-1-carbonyl]pyrrolidine-3-carboxamide;
[6-[(3-Methylsulfonylphenyl)methyl]-2-azaspiro[3.3]heptan-2-yl]-[(3S)-3-(tetrazol-1-yl)pyrrolidin-1-yl]methanone;
[3-[[2-Chloro-4-(trifluoromethyl)phenyl]methylamino]azetidin-1-yl]-[(3S)-3-(tetrazol-1-yl)pyrrolidin-1-yl]methanone;
[3-[[3-Fluoro-4-(trifluoromethyl)phenyl]methylamino]azetidin-1-yl]-[(3S)-3-(tetrazol-1-yl)pyrrolidin-1-yl]methanone;
[3-[[2-Fluoro-4-(trifluoromethoxy)phenyl]methylamino]azetidin-1-yl]-[(3S)-3-(tetrazol-1-yl)pyrrolidin-1-yl]methanone;
[3-[[5-Chloro-2-(trifluoromethyl)phenyl]methylamino]azetidin-1-yl]-[(3S)-3-(tetrazol-1-yl)pyrrolidin-1-yl]methanone;
[3-[[3-Chloro-5-(trifluoromethyl)phenyl]methylamino]azetidin-1-yl]-[(3S)-3-(tetrazol-1-yl)pyrrolidin-1-yl]methanone;
[(3S)-3-(Tetrazol-1-yl)pyrrolidin-1-yl]-[3-[5-[1-(trifluoromethyl)cyclopropyl]-2-pyridyl]azetidin-1-yl]methanone;
(3S)-1-[3-[6-(2-Azaspiro[3.4]octan-2-yl)-3-pyridyl]azetidine-1-carbonyl]pyrrolidine-3-carboxamide;
1-[3-[6-(2-Azaspiro[3.4]octan-2-yl)-3-pyridyl]azetidine-1-carbonyl]pyrrolidine-3-sulfonamide;
1-[4-[1-[(3S)-3-(1H-1,2,4-Triazol-5-yl)pyrrolidine-1-carbonyl]azetidin-3-yl]phenyl]piperidine-2-carboxamide;
(3S)-1-[7-[[6-(Trifluoromethyl)-3-pyridyl]methyl]-2-azaspiro[3.5]nonane-2-carbonyl]pyrrolidine-3-carboxamide;
2-[4-[1-[(3S)-3-(1H-1,2,4-Triazol-5-yl)pyrrolidine-1-carbonyl]azetidin-3-yl]phenyl]benzamide;
(3S)-1-[3-[2-Fluoro-4-(trifluoromethyl)phenyl]methyl-methyl-amino]azetidine-1-carbonyl]pyrrolidine-3-carboxamide;
(3S)-1-[3-[4-(Trifluoromethylsulfonyl)phenyl]methoxy]azetidine-1-carbonyl]pyrrolidine-3-carboxamide;
(3S)-1-[3-[3-(Trifluoromethylsulfonyl)phenyl]methoxy]azetidine-1-carbonyl]pyrrolidine-3-carboxamide;
(3S)-1-[3-[5-[6-(Trifluoromethyl)-3-pyridyl]-1,2,4-oxadiazol-3-yl]azetidine-1-carbonyl]pyrrolidine-3-carboxamide;
(3S)-1-[3-[5-[3-(Trifluoromethyl)-1-bicyclo[1.1.1]pentanyl]-1,2,4-oxadiazol-3-yl]azetidine-1-carbonyl]pyrrolidine-3-carboxamide;
rac-(3S)-1-[3-[6-[3-(Trifluoromethyl)pyrrolidin-1-yl]-3-pyridyl]azetidine-1-carbonyl]pyrrolidine-3-carboxamide;
N-Ethyl-2-[4-[1-[(3S)-3-(1H-1,2,4-triazol-5-yl)pyrrolidine-1-carbonyl]azetidin-3-yl]phenyl]benzamide;
[3-[Cyclopropylmethyl-[[2-fluoro-4-(trifluoromethyl)phenyl]methyl]amino]azetidin-1-yl]-[(3S)-3-(tetrazol-1-yl)pyrrolidin-1-yl]methanone;
[3-[4-(4-Chloro-2-methylsulfonyl-phenyl)phenyl]azetidin-1-yl]-(3-hydroxyazetidin-1-yl)methanone;
[3-[4-(4-Chloro-2-methylsulfonyl-phenyl)phenyl]azetidin-1-yl]-(3-hydroxy-3-methyl-azetidin-1-yl)methanone;
1-[3-[4-(4-Chloro-2-methylsulfonyl-phenyl)phenyl]azetidine-1-carbonyl]azetidine-3-carboxamide;
1-[3-[4-[5-(Trifluoromethyl)pyrimidin-2-yl]oxyphenyl]azetidine-1-carbonyl]azetidine-3-carboxamide;
[3-[2-Fluoro-4-(trifluoromethyl)phenyl]methyl-methyl-amino]azetidin-1-yl]-[(3S)-3-(tetrazol-1-yl)pyrrolidin-1-yl]methanone;
(3S)-1-[3-(5-Spiro[3.3]heptan-2-ylpyrazin-2-yl)azetidine-1-carbonyl]pyrrolidine-3-carboxamide;
(3S)-1-[3-[4-[4-Methyl-2-(pyrrolidine-1-carbonyl)phenyl]phenyl]azetidine-1-carbonyl]pyrrolidine-3-carboxamide;
(3S)-1-[6-[[6-(Trifluoromethyl)-3-pyridyl]methyl]-2-azaspiro[3.3]heptane-2-carbonyl]pyrrolidine-3-carboxamide;
[3-[4-(4-Chloro-2-methylsulfonyl-phenyl)phenyl]azetidin-1-yl]-[3-(methylsulfonimidoyl)pyrrolidin-1-yl]methanone;
[3-[[2-Fluoro-4-(trifluoromethyl)phenyl]methyl-methyl-amino]azetidin-1-yl]-[(3S)-3-(1,2,4-triazol-4-yl)pyrrolidin-1-yl]methanone;
N-[1-[3-[4-[1-(Trifluoromethyl)cyclopropyl]phenyl]azetidine-1-carbonyl]azetidin-3-yl]methanesulfonamide;
N-[1-[3-[4-[[1-(Trifluoromethyl)cyclopropyl]methoxy]phenyl]azetidine-1-carbonyl]azetidin-3-yl]methanesulfonamide;
N-[1-[3-[4-[5-(Trifluoromethyl)pyrazin-2-yl]oxyphenyl]azetidine-1-carbonyl]azetidin-3-yl]methanesulfonamide;
N-[1-[3-[6-[3-(Trifluoromethyl)azetidin-1-yl]-3-pyridyl]azetidine-1-carbonyl]azetidin-3-yl]methanesulfonamide;
(3S)-1-[3-[3-[5-(2,2-Dimethylpropyl)-1,3,4-oxadiazol-2-yl]-1-bicyclo[1.1.1]pentanyl]azetidine-1-carbonyl]pyrrolidine-3-carboxamide;
[3-[3-[5-(2,2-Dimethylpropyl)-1,3,4-oxadiazol-2-yl]-1-bicyclo[1.1.1]pentanyl]azetidin-1-yl]-[(3S)-3-(1H-1,2,4-triazol-5-yl)pyrrolidin-1-yl]methanone;
(3S)-1-[3-[4-[3-(2,2-Dimethylpropyl)triazol-4-yl]phenyl]azetidine-1-carbonyl]pyrrolidine-3-carboxamide;
rac-(3S)-1-[3-[6-(2,2-Difluoro-5-azaspiro[2.4]heptan-5-yl)-3-pyridyl]azetidine-1-carbonyl]pyrrolidine-3-carboxamide;
[(3S)-3-(Tetrazol-1-yl)pyrrolidin-1-yl]-[6-[[6-(trifluoromethyl)pyridazin-3-yl]methyl]-2,6-diazaspiro[3.3]heptan-2-yl]methanone;
tert-Butyl 2-[3-[[2-fluoro-4-(trifluoromethyl)phenyl]methyl-methyl-amino]azetidine-1-carbonyl]-2,8-diazaspiro[3.5]nonane-8-carboxylate;
N-[1-[2-[[2-Fluoro-4-(trifluoromethyl)phenyl]methyl]-2,7-diazaspiro[3.5]nonane-7-carbonyl]azetidin-3-yl]methanesulfonamide;
(3S)-1-[3-[6-(6-Oxa-2-azaspiro[3.4]octan-2-yl)-3-pyridyl]azetidine-1-carbonyl]pyrrolidine-3-carboxamide;
(3S)-1-[3-[6-(5-Oxa-2-azaspiro[3.4]octan-2-yl)-3-pyridyl]azetidine-1-carbonyl]pyrrolidine-3-carboxamide;
[7-[6-(Difluoromethoxy)-3-pyridyl]methyl]-2-azaspiro[3.5]nonan-2-yl]-[(3S)-3-(tetrazol-1-yl)pyrrolidin-1-yl]methanone;
[6-[(2,4-Difluorophenyl)methyl]-2-azaspiro[3.3]heptan-2-yl]-[(3S)-3-(tetrazol-1-yl)pyrrolidin-1-yl]methanone;
[6-[[2-Fluoro-4-(trifluoromethyl)phenyl]methyl]-2-azaspiro[3.3]heptan-2-yl]-[(3S)-3-(tetrazol-1-yl)pyrrolidin-1-yl]methanone;
[6-[[4-Fluoro-2-(trifluoromethyl)phenyl]methyl]-2-azaspiro[3.3]heptan-2-yl]-[(3S)-3-(tetrazol-1-yl)pyrrolidin-1-yl]methanone;
N-[1-[3-[4-[1-(Trifluoromethyl)cyclopropyl]phenyl]azetidine-1-carbonyl]azetidin-3-yl]acetamide;
N-[1-[3-[[2-Fluoro-4-(trifluoromethyl)phenyl]methoxy]azetidine-1-carbonyl]azetidin-3-yl]acetamide;
N-[1-[7-[1-(Trifluoromethyl)cyclopropyl]methoxy]-2-azaspiro[3.5]nonane-2-carbonyl]azetidin-3-yl]acetamide;
N-[1-[3-[4-[5-(Trifluoromethyl)pyrazin-2-yl]oxyphenyl]azetidine-1-carbonyl]azetidin-3-yl]acetamide;
(3S)-1-[3-[6-[(3R)-3-(Trifluoromethyl)pyrrolidin-1-yl]-3-pyridyl]azetidine-1-carbonyl]pyrrolidine-3-carboxamide;
(3S)-1-[3-[6-[(3S)-3-(Trifluoromethyl)pyrrolidin-1-yl]-3-pyridyl]azetidine-1-carbonyl]pyrrolidine-3-carboxamide;
(3S)-1-[3-[4-[5-(Trifluoromethyl)pyrazin-2-yl]oxyphenyl]azetidine-1-carbonyl]pyrrolidine-3-carboxamide;
(3S)-1-[3-[5-[1-(Trifluoromethyl)cyclopropyl]methylamino]-2-pyridyl]azetidine-1-carbonyl]pyrrolidine-3-carboxamide;
(3S)-1-[3-[5-[1-(Trifluoromethyl)cyclopropyl]methylamino]pyrazin-2-yl]azetidine-1-carbonyl]pyrrolidine-3-carboxamide;
(3S)-1-[3-[6-[3-(Trifluoromethyl)azetidin-1-yl]-3-pyridyl]azetidine-1-carbonyl]pyrrolidine-3-carboxamide;
(3S)-1-[3-[2-[3-(Trifluoromethyl)azetidin-1-yl]pyrimidin-5-yl]azetidine-1-carbonyl]pyrrolidine-3-carboxamide;
(3S)-1-[7-[(3,5-Difluoro-2-pyridyl)methyl]-2-azaspiro[3.5]nonane-2-carbonyl]pyrrolidine-3-carboxamide;
(3S)-1-[7-[(5-Chloro-2-pyridyl)methyl]-2-azaspiro[3.5]nonane-2-carbonyl]pyrrolidine-3-carboxamide;
(3S)-1-[7-[5-(Trifluoromethyl)pyrazin-2-yl]methyl]-2-azaspiro[3.5]nonane-2-carbonyl]pyrrolidine-3-carboxamide;
(3S)-1-[7-[5-(Trifluoromethyl)-2-pyridyl]methyl]-2-azaspiro[3.5]nonane-2-carbonyl]pyrrolidine-3-carboxamide;
(3S)-1-[7-[6-(Difluoromethoxy)-3-pyridyl]methyl]-2-azaspiro[3.5]nonane-2-carbonyl]pyrrolidine-3-carboxamide;
(3S)-1-[6-[6-(Trifluoromethyl)pyridazin-3-yl]methyl]-2-azaspiro[3.3]heptane-2-carbonyl]pyrrolidine-3-carboxamide;
(3S)-1-[6-[2-(Trifluoromethyl)pyrimidin-5-yl]methyl]-2-azaspiro[3.3]heptane-2-carbonyl]pyrrolidine-3-carboxamide;
(3S)-1-[(6S)-6-[5-(Trifluoromethyl)pyrazin-2-yl]oxy-2-azaspiro[3.4]octane-2-carbonyl]pyrrolidine-3-carboxamide;
[6-(4-Fluorophenyl)-2-azaspiro[3.3]heptan-2-yl]-[(3S)-3-(tetrazol-1-yl)pyrrolidin-1-yl]methanone;
[(3S)-3-(Tetrazol-1-yl)pyrrolidin-1-yl]-[6-[[4-(trifluoromethylsulfonyl)phenyl]methyl]-2,6-diazaspiro[3.3]heptan-2-yl]methanone;
(3S)-1-[3-[4-(5-Cyclobutyl-4H-1,2,4-triazol-3-yl)phenyl]azetidine-1-carbonyl]pyrrolidine-3-carboxamide;
(3S)-1-[6-(3-Cyclopropyl-1,2,4-triazol-1-yl)-2-azaspiro[3.3]heptane-2-carbonyl]pyrrolidine-3-carboxamide;
[6-[(2,4-Difluorophenyl)methyl]-2-azaspiro[3.3]heptan-2-yl]-[(3S)-3-(triazol-1-yl)pyrrolidin-1-yl]methanone;
[3-[6-(2-Chlorophenoxy)-3-pyridyl]azetidin-1-yl]-[(3R)-3-(triazol-1-yl)pyrrolidin-1-yl]methanone;
[3-[4-(4-Chloro-2-methylsulfonyl-phenyl)phenyl]azetidin-1-yl]-[(3R)-3-(triazol-1-yl)pyrrolidin-1-yl]methanone;
[6-[(2,4-Difluorophenyl)methyl]-2-azaspiro[3.3]heptan-2-yl]-[(3R)-3-(triazol-1-yl)pyrrolidin-1-yl]methanone;
[3-[4-(4-Chloro-2-methylsulfonyl-phenyl)phenyl]azetidin-1-yl]-[(3R)-3-(triazol-2-yl)pyrrolidin-1-yl]methanone;
[6-[(2,4-Difluorophenyl)methyl]-2-azaspiro[3.3]heptan-2-yl]-[(3R)-3-(triazol-2-yl)pyrrolidin-1-yl]methanone;
[3-[[2-Fluoro-4-(trifluoromethyl)phenyl]methyl-methyl-amino]azetidin-1-yl]-[(3R)-3-(triazol-2-yl)pyrrolidin-1-yl]methanone;
[3-[4-(4-Chloro-2-methylsulfonyl-phenyl)phenyl]azetidin-1-yl]-[(3S)-3-(triazol-1-yl)pyrrolidin-1-yl]methanone;
[7-[[6-(Difluoromethoxy)-3-pyridyl]methyl]-2-azaspiro[3.5]nonan-2-yl]-[(3R)-3-(triazol-1-yl)pyrrolidin-1-yl]methanone;
[3-[4-(4-Chloro-2-methylsulfonyl-phenyl)phenyl]azetidin-1-yl]-[(3S)-3-(triazol-2-yl)pyrrolidin-1-yl]methanone;
[3-[4-(4-Chloro-2-methylsulfonyl-phenyl)phenyl]azetidin-1-yl]-[6-(triazol-2-yl)-2-azaspiro[3.3]heptan-2-yl]methanone;
[3-[4-(4-Chloro-2-methylsulfonyl-phenyl)phenyl]azetidin-1-yl]-[6-(triazol-1-yl)-2-azaspiro[3.3]heptan-2-yl]methanone;
(3S)-1-[3-[4-(3-Cyclopropyl-5-methyl-pyrazol-1-yl)phenyl]azetidine-1-carbonyl]pyrrolidine-3-carboxamide;
(3S)-1-[3-[4-[2-Methylsulfonyl-4-(trifluoromethyl)phenyl]phenyl]azetidine-1-carbonyl]pyrrolidine-3-carboxamide;
(3S)-1-[7-(4-Chloro-2-methylsulfonyl-phenyl)-2-azaspiro[3.5]nonane-2-carbonyl]pyrrolidine-3-carboxamide;
[3-[[2-Fluoro-4-(trifluoromethyl)phenyl]methoxy]azetidin-1-yl]-[(3S)-3-(1H-1,2,4-triazol-5-yl)pyrrolidin-1-yl]methanone;
(3S)-1-[3-[4-(5-Cyclopropyl-4H-1,2,4-triazol-3-yl)phenyl]azetidine-1-carbonyl]pyrrolidine-3-carboxamide;
(3S)-1-[3-[4-[5-[1-(Trifluoromethyl)cyclopropyl]-4H-1,2,4-triazol-3-yl]phenyl]azetidine-1-carbonyl]pyrrolidine-3-carboxamide;
(3S)-1-[6-[[5-(Trifluoromethyl)pyrimidin-2-yl]methyl]-2-azaspiro[3.3]heptane-2-carbonyl]pyrrolidine-3-carboxamide;
[(3S)-3-(1H-1,2,4-Triazol-5-yl)pyrrolidin-1-yl]-[6-[5-(trifluoromethyl)pyrimidin-2-yl]methyl]-2-azaspiro[3.3]heptan-2-yl]methanone;
(3S)-1-[6-[[5-(Trifluoromethoxy)-2-pyridyl]methyl]-2-azaspiro[3.3]heptane-2-carbonyl]pyrrolidine-3-carboxamide;
[(3S)-3-(1H-1,2,4-Triazol-5-yl)pyrrolidin-1-yl]-[6-[5-(trifluoromethoxy)-2-pyridyl]methyl]-2-azaspiro[3.3]heptan-2-yl]methanone;
rac-(3S)-1-[6-[6-(Trifluoromethyl)-3-pyridyl]methyl]-2-azaspiro[3.4]octane-2-carbonyl]pyrrolidine-3-carboxamide;
[rac-(3S)-3-(1H-1,2,4-Triazol-5-yl)pyrrolidin-1-yl]-[6-[6-(trifluoromethyl)-3-pyridyl]methyl]-2-azaspiro[3.4]octan-2-yl]methanone;
(3S)-1-[7-(4-Chloro-2-fluoro-phenyl)-2,7-diazaspiro[3.5]nonane-2-carbonyl]pyrrolidine-3-carboxamide;
(3S)-1-[7-(5-Chloro-3-fluoro-2-pyridyl)-2,7-diazaspiro[3.5]nonane-2-carbonyl]pyrrolidine-3-carboxamide;
1-[4-[1-[(3S)-3-(1H-1,2,4-Triazol-5-yl)pyrrolidine-1-carbonyl]azetidin-3-yl]phenyl]-4-(trifluoromethyl)pyridin-2-one;
[3-[4-(3-Cyclopropyl-5-methyl-pyrazol-1-yl)phenyl]azetidin-1-yl]-[(3S)-3-(1H-1,2,4-triazol-5-yl)pyrrolidin-1-yl]methanone;
[(3S)-3-(1H-1,2,4-Triazol-5-yl)pyrrolidin-1-yl]-[3-[6-[(3R)-3-(trifluoromethyl)pyrrolidin-1-yl]-3-pyridyl]azetidin-1-yl]methanone;
[(3S)-3-(1H-1,2,4-Triazol-5-yl)pyrrolidin-1-yl]-[3-[6-[(3S)-3-(trifluoromethyl)pyrrolidin-1-yl]-3-pyridyl]azetidin-1-yl]methanone;
[(3S)-3-(1H-1,2,4-Triazol-5-yl)pyrrolidin-1-yl]-[3-[6-[3-(trifluoromethyl)azetidin-1-yl]-3-pyridyl]azetidin-1-yl]methanone;
[(3S)-3-(1H-1,2,4-Triazol-5-yl)pyrrolidin-1-yl]-[3-[2-[3-(trifluoromethyl)azetidin-1-yl]pyrimidin-5-yl]azetidin-1-yl]methanone;
[3-[4-(2-Chloro-4-methylsulfonyl-phenyl)phenyl]azetidin-1-yl]-[(3S)-3-(1H-1,2,4-triazol-5-yl)pyrrolidin-1-yl]methanone;
[3-[5-(4-Chloro-2-methylsulfonyl-phenyl)-2-pyridyl]azetidin-1-yl]-[(3S)-3-(1H-1,2,4-triazol-5-yl)pyrrolidin-1-yl]methanone;
[7-[(5-Fluoro-2-pyridyl)methyl]-2-azaspiro[3.5]nonan-2-yl]-[(3S)-3-(1H-1,2,4-triazol-5-yl)pyrrolidin-1-yl]methanone;
[(3S)-3-(1H-1,2,4-Triazol-5-yl)pyrrolidin-1-yl]-[3-[5-[[1-(trifluoromethyl)cyclopropyl]methylamino]pyrazin-2-yl]azetidin-1-yl]methanone;
[7-[(5-Chloro-2-pyridyl)methyl]-2-azaspiro[3.5]nonan-2-yl]-[(3S)-3-(1H-1,2,4-triazol-5-yl)pyrrolidin-1-yl]methanone;
[(3S)-3-(1H-1,2,4-Triazol-5-yl)pyrrolidin-1-yl]-[7-[5-(trifluoromethyl)pyrazin-2-yl]methyl]-2-azaspiro[3.5]nonan-2-yl]methanone;
[7-[6-(Difluoromethoxy)-3-pyridyl]methyl]-2-azaspiro[3.5]nonan-2-yl]-[(3S)-3-(1H-1,2,4-triazol-5-yl)pyrrolidin-1-yl]methanone;
[(3S)-3-(1H-1,2,4-Triazol-5-yl)pyrrolidin-1-yl]-[7-[1-(trifluoromethyl)cyclopropyl]methoxy]-2-azaspiro[3.5]nonan-2-yl]methanone;
[(3S)-3-(1H-1,2,4-Triazol-5-yl)pyrrolidin-1-yl]-[7-[[4-(trifluoromethylsulfonyl)phenyl]methyl]-2,7-diazaspiro[3.5]nonan-2-yl]methanone;
[(3S)-3-(1H-1,2,4-Triazol-5-yl)pyrrolidin-1-yl]-[7-[[3-(trifluoromethylsulfonyl)phenyl]methyl]-2,7-diazaspiro[3.5]nonan-2-yl]methanone;
[6-[[3-Fluoro-5-(trifluoromethyl)-2-pyridyl]methyl]-2-azaspiro[3.3]heptan-2-yl]-[(3S)-3-(1H-1,2,4-triazol-5-yl)pyrrolidin-1-yl]methanone;
[(3S)-3-(1H-1,2,4-Triazol-5-yl)pyrrolidin-1-yl]-[6-[[6-(trifluoromethyl)pyridazin-3-yl]methyl]-2-azaspiro[3.3]heptan-2-yl]methanone;
[(3S)-3-(1H-1,2,4-Triazol-5-yl)pyrrolidin-1-yl]-[6-[2-(trifluoromethyl)pyrimidin-5-yl]methyl]-2-azaspiro[3.3]heptan-2-yl]methanone;
[6-[4-Fluoro-2-(trifluoromethyl)phenyl]methyl]-2-azaspiro[3.3]heptan-2-yl]-[(3S)-3-(1H-1,2,4-triazol-5-yl)pyrrolidin-1-yl]methanone;
[6-[[2-Fluoro-4-(trifluoromethyl)phenyl]methyl]-2-azaspiro[3.3]heptan-2-yl]-[(3S)-3-(1H-1,2,4-triazol-5-yl)pyrrolidin-1-yl]methanone;
[6-[3-Fluoro-5-(trifluoromethyl)phenyl]methyl]-2-azaspiro[3.3]heptan-2-yl]-[(3S)-3-(1H-1,2,4-triazol-5-yl)pyrrolidin-1-yl]methanone;
[(3S)-3-(1H-1,2,4-Triazol-5-yl)pyrrolidin-1-yl]-[(6S)-6-[[5-(trifluoromethyl)pyrazin-2-yl]methyl]-2-azaspiro[3.4]octan-2-yl]methanone;
[(3S)-3-(1H-1,2,4-Triazol-5-yl)pyrrolidin-1-yl]-[(6R)-6-[[5-(trifluoromethyl)pyrazin-2-yl]methyl]-2-azaspiro[3.4]octan-2-yl]methanone;
[6-[(4-Methylsulfonylphenyl)methyl]-2-azaspiro[3.4]octan-2-yl]-[rac-(3S)-3-(1H-1,2,4-triazol-5-yl)pyrrolidin-1-yl]methanone;
[(3S)-3-(1H-1,2,4-Triazol-5-yl)pyrrolidin-1-yl]-[(6S)-6-[5-(trifluoromethyl)pyrazin-2-yl]oxy-2-azaspiro[3.4]octan-2-yl]methanone;
[(3S)-3-(1H-1,2,4-Triazol-5-yl)pyrrolidin-1-yl]-[3-[1-[[4-(trifluoromethyl)phenyl]methyl]azetidin-3-yl]azetidin-1-yl]methanone;
[(3S)-3-(1H-1,2,4-Triazol-5-yl)pyrrolidin-1-yl]-[6-[[5-(trifluoromethyl)-3-pyridyl]methyl]-2-azaspiro[3.3]heptan-2-yl]methanone;
[(3S)-3-(1H-1,2,4-Triazol-5-yl)pyrrolidin-1-yl]-[6-[[4-(trifluoromethyl)-2-pyridyl]methyl]-2-azaspiro[3.3]heptan-2-yl]methanone;
[(3S)-3-(1H-1,2,4-Triazol-5-yl)pyrrolidin-1-yl]-[7-[[5-(trifluoromethyl)-2-pyridyl]methyl]-2,7-diazaspiro[3.4]octan-2-yl]methanone;
[(3S)-3-(1H-1,2,4-Triazol-5-yl)pyrrolidin-1-yl]-[3-[3-[[1-(trifluoromethyl)cyclopropyl]methylamino]-1-bicyclo[1.1.1]pentanyl]azetidin-1-yl]methanone;
[(3S)-3-(1H-1,2,4-Triazol-5-yl)pyrrolidin-1-yl]-[3-[3-[1-(trifluoromethyl)cyclopropyl]amino]methyl]-1-bicyclo[1.1.1]pentanyl]azetidin-1-yl]methanone;
[3-[3-(5-Cyclopropyl-1,3,4-oxadiazol-2-yl)-1-bicyclo[1.1.1]pentanyl]azetidin-1-yl]-[(3S)-3-(1H-1,2,4-triazol-5-yl)pyrrolidin-1-yl]methanone;
[(3S)-3-(1H-1,2,4-Triazol-5-yl)pyrrolidin-1-yl]-[3-[3-[5-(2,2,2-trifluoroethyl)-1,3,4-oxadiazol-2-yl]-1-bicyclo[1.1.1]pentanyl]azetidin-1-yl]methanone;
(4S)-1-[4-[1-[(3S)-3-(1H-1,2,4-Triazol-5-yl)pyrrolidine-1-carbonyl]azetidin-3-yl]phenyl]-4-(trifluoromethyl) piperidin-2-one;
(4R)-1-[4-[1-[(3S)-3-(1H-1,2,4-Triazol-5-yl)pyrrolidine-1-carbonyl]azetidin-3-yl]phenyl]-4-(trifluoromethyl) piperidin-2-one;
[3-[4-[3-(Difluoromethyl)-5-methyl-pyrazol-1-yl]phenyl]azetidin-1-yl]-[(3S)-3-(1H-1,2,4-triazol-5-yl)pyrrolidin-1-yl]methanone;
[3-[4-[5-Methyl-3-(trifluoromethyl)pyrazol-1-yl]phenyl]azetidin-1-yl]-[(3S)-3-(1H-1,2,4-triazol-5-yl)pyrrolidin-1-yl]methanone;
[3-[4-(3-tert-Butyl-5-methyl-pyrazol-1-yl)phenyl]azetidin-1-yl]-[(3S)-3-(1H-1,2,4-triazol-5-yl)pyrrolidin-1-yl]methanone;
(3S)-1-[6-[3-Fluoro-5-(trifluoromethyl)-2-pyridyl]methyl]-2-azaspiro[3.3]heptane-2-carbonyl]pyrrolidine-3-carboxamide;
(3S)-1-[7-[5-(Trifluoromethyl)-2-pyridyl]methyl]-2,7-diazaspiro[3.4]octane-2-carbonyl]pyrrolidine-3-carboxamide;
(3S)-1-[3-[3-[5-(2,2,2-Trifluoroethyl)-1,3,4-oxadiazol-2-yl]-1-bicyclo[1.1.1]pentanyl]azetidine-1-carbonyl]pyrrolidine-3-carboxamide;
[6-[[4-Methylsulfonyl-3-(trifluoromethyl)phenyl]methyl]-2-azaspiro[3.3]heptan-2-yl]-[(3S)-3-(1H-1,2,4-triazol-5-yl)pyrrolidin-1-yl]methanone;
[6-[[2-Methylsulfonyl-4-(trifluoromethyl)phenyl]methyl]-2-azaspiro[3.3]heptan-2-yl]-[(3S)-3-(1H-1,2,4-triazol-5-yl)pyrrolidin-1-yl]methanone;
[6-[[3-Methylsulfonyl-4-(trifluoromethyl)phenyl]methyl]-2-azaspiro[3.3]heptan-2-yl]-[(3S)-3-(1H-1,2,4-triazol-5-yl)pyrrolidin-1-yl]methanone;
[rac-(3S)-3-(1H-1,2,4-Triazol-5-yl)pyrrolidin-1-yl]-[6-[[3-(trifluoromethylsulfonimidoyl)phenyl]methyl]-2-azaspiro[3.3]heptan-2-yl]methanone;
[rac-(3S)-3-(1H-1,2,4-Triazol-5-yl)pyrrolidin-1-yl]-[6-[[4-(trifluoromethylsulfonimidoyl)phenyl]methyl]-2-azaspiro[3.3]heptan-2-yl]methanone;
[(3S)-3-(1H-1,2,4-Triazol-5-yl)pyrrolidin-1-yl]-[6-[4-(trifluoromethyl)pyrazol-1-yl]methyl]-2-azaspiro[3.3]heptan-2-yl]methanone;
[(3S)-3-(1H-1,2,4-Triazol-5-yl)pyrrolidin-1-yl]-[3-[4-[5-[1-(trifluoromethyl)cyclopropyl]-4H-1,2,4-triazol-3-yl]phenyl]azetidin-1-yl]methanone;
(3S)-1-[3-[3-[[1-(Trifluoromethyl)cyclopropyl]methylamino]-1-bicyclo[1.1.1]pentanyl]azetidine-1-carbonyl]pyrrolidine-3-carboxamide;
(3S)-1-[3-[3-[[1-(Trifluoromethyl)cyclopropyl]amino]methyl]-1-bicyclo[1.1.1]pentanyl]azetidine-1-carbonyl]pyrrolidine-3-carboxamide;
rac-(1S,4R,5R)-2-[3-[4-(4-Chloro-2-methylsulfonyl-phenyl)phenyl]azetidine-1-carbonyl]-2-azabicyclo[2.1.1]hexane-5-carboxamide;
(3S)-1-[3-[3-(5-Cyclopropyl-1,3,4-oxadiazol-2-yl)-1-bicyclo[1.1.1]pentanyl]azetidine-1-carbonyl]pyrrolidine-3-carboxamide;
(3S)-1-[7-(4-Chloro-2-methylsulfonyl-phenyl)-2,7-diazaspiro[3.5]nonane-2-carbonyl]pyrrolidine-3-carboxamide;
[6-[(5-Methylsulfonyl-2-pyridyl)methyl]-2-azaspiro[3.3]heptan-2-yl]-[(3S)-3-(1H-1,2,4-triazol-5-yl)pyrrolidin-1-yl]methanone;
[3-[4-[3-(2,2-Dimethylpropyl)triazol-4-yl]phenyl]azetidin-1-yl]-[(3S)-3-(1H-1,2,4-triazol-5-yl)pyrrolidin-1-yl]methanone;
[(3S)-3-(1H-1,2,4-Triazol-5-yl)pyrrolidin-1-yl]-[6-[[3-(trifluoromethyl)-1,2,4-triazol-1-yl]methyl]-2-azaspiro[3.3]heptan-2-yl]methanone;
[(3S)-3-(1H-1,2,4-Triazol-5-yl)pyrrolidin-1-yl]-[6-[4-(trifluoromethyl) oxazol-2-yl]methyl]-2-azaspiro[3.3]heptan-2-yl]methanone;
[(3S)-3-(1H-1,2,4-Triazol-5-yl)pyrrolidin-1-yl]-[6-[[3-(trifluoromethyl)-1,2,4-thiadiazol-5-yl]methyl]-2-azaspiro[3.3]heptan-2-yl]methanone;
[(3S)-3-(1H-1,2,4-Triazol-5-yl)pyrrolidin-1-yl]-[3-[3-[5-(trifluoromethyl)pyrazin-2-yl]oxy-1-bicyclo[1.1.1]pentanyl]azetidin-1-yl]methanone;
[(3S)-3-(1H-1,2,4-Triazol-5-yl)pyrrolidin-1-yl]-[6-[[4-(trifluoromethyl) imidazol-1-yl]methyl]-2-azaspiro[3.3]heptan-2-yl]methanone;
[3-[4-(3,5-Diisopropylpyrazol-1-yl)phenyl]azetidin-1-yl]-[(3S)-3-(1H-1,2,4-triazol-5-yl)pyrrolidin-1-yl]methanone;
[3-[4-(3,5-Dimethyl-1,2,4-triazol-1-yl)phenyl]azetidin-1-yl]-[(3S)-3-(1H-1,2,4-triazol-5-yl)pyrrolidin-1-yl]methanone;
[(3S)-3-(4H-1,2,4-Triazol-3-yl)pyrrolidin-1-yl]-[7-[5-(trifluoromethyl)-2-pyridyl]amino]-2-azaspiro[3.5]nonan-2-yl]methanone;
[6-[(3-Fluoro-5-methylsulfonyl-phenyl)methyl]-2-azaspiro[3.3]heptan-2-yl]-[(3S)-3-(4H-1,2,4-triazol-3-yl)pyrrolidin-1-yl]methanone;
[6-[[4-Methylsulfonyl-3-(trifluoromethyl)phenyl]methyl]-2-azaspiro[3.4]octan-2-yl]-[rac-(3S)-3-(4H-1,2,4-triazol-3-yl)pyrrolidin-1-yl]methanone;
[3-[[2-Fluoro-4-(trifluoromethylsulfonyl)phenyl]methoxy]azetidin-1-yl]-[(3S)-3-(4H-1,2,4-triazol-3-yl)pyrrolidin-1-yl]methanone;
[(3S)-3-(4H-1,2,4-Triazol-3-yl)pyrrolidin-1-yl]-[7-[6-(trifluoromethyl)-3-pyridyl]methyl]-2-azaspiro[3.5]nonan-2-yl]methanone;
[3-[6-(2-Chlorophenoxy)-3-pyridyl]azetidin-1-yl]-[(3S)-3-(4H-1,2,4-triazol-3-yl)pyrrolidin-1-yl]methanone;
[6-[(3,5-Difluoro-2-pyridyl)methyl]-2-azaspiro[3.3]heptan-2-yl]-[(3S)-3-(4H-1,2,4-triazol-3-yl)pyrrolidin-1-yl]methanone;
[7-[Methyl-[5-(trifluoromethyl)pyrazin-2-yl]amino]-2-azaspiro[3.5]nonan-2-yl]-[(3S)-3-(4H-1,2,4-triazol-3-yl)pyrrolidin-1-yl]methanone;
[6-[2-Methylsulfonyl-4-(trifluoromethyl)phenyl]-2-azaspiro[3.3]heptan-2-yl]-[(3S)-3-(4H-1,2,4-triazol-3-yl)pyrrolidin-1-yl]methanone;
[(3S)-3-(4H-1,2,4-Triazol-3-yl)pyrrolidin-1-yl]-[3-[[4-(trifluoromethylsulfonyl)phenyl]methoxy]azetidin-1-yl]methanone;
[6-(4-Methylsulfonylphenyl)-2-azaspiro[3.3]heptan-2-yl]-[(3S)-3-(4H-1,2,4-triazol-3-yl)pyrrolidin-1-yl]methanone;
[(3S)-3-(4H-1,2,4-Triazol-3-yl)pyrrolidin-1-yl]-[6-[4-(trifluoromethylsulfonyl)phenyl]-2-azaspiro[3.3]heptan-2-yl]methanone;
[(3S)-3-(4H-1,2,4-Triazol-3-yl)pyrrolidin-1-yl]-[3-[4-[2-(trifluoromethyl)pyrimidin-5-yl]oxyphenyl]azetidin-1-yl]methanone;
[(3S)-3-(4H-1,2,4-Triazol-3-yl)pyrrolidin-1-yl]-[7-[[5-(trifluoromethyl)pyrazin-2-yl]amino]-2-azaspiro[3.5]nonan-2-yl]methanone;
[7-(5-Chloro-2-methylsulfonyl-phenoxy)-2-azaspiro[3.5]nonan-2-yl]-[(3S)-3-(4H-1,2,4-triazol-3-yl)pyrrolidin-1-yl]methanone;
[3-[4-(3,5-Dimethylpyrazol-1-yl)phenyl]azetidin-1-yl]-[(3S)-3-(4H-1,2,4-triazol-3-yl)pyrrolidin-1-yl]methanone;
[3-[6-(N-Methylanilino)-3-pyridyl]azetidin-1-yl]-[(3S)-3-(4H-1,2,4-triazol-3-yl)pyrrolidin-1-yl]methanone;
2-Methoxy-4-[[2-[(3S)-3-(4H-1,2,4-triazol-3-yl)pyrrolidine-1-carbonyl]-2-azaspiro[3.3]heptan-6-yl]methyl]benzonitrile;
[3-[4-[1-Methyl-3-(trifluoromethyl)pyrazol-4-yl]phenyl]azetidin-1-yl]-[(3S)-3-(4H-1,2,4-triazol-3-yl)pyrrolidin-1-yl]methanone;
[3-[6-(4-Fluorophenoxy)-3-pyridyl]azetidin-1-yl]-[(3S)-3-(4H-1,2,4-triazol-3-yl)pyrrolidin-1-yl]methanone;
[3-(4-Cyclopropyl-2-fluoro-phenoxy)azetidin-1-yl]-[(3S)-3-(4H-1,2,4-triazol-3-yl)pyrrolidin-1-yl]methanone;
[3-(4-Chloro-3-cyclopropyl-phenoxy)azetidin-1-yl]-[(3S)-3-(4H-1,2,4-triazol-3-yl)pyrrolidin-1-yl]methanone;
[3-[4-[5-[(1-Methylcyclopropyl)methyl]-4H-1,2,4-triazol-3-yl]phenyl]azetidin-1-yl]-[(3S)-3-(4H-1,2,4-triazol-3-yl)pyrrolidin-1-yl]methanone;
[(3S)-3-(1H-1,2,4-Triazol-5-yl)pyrrolidin-1-yl]-[6-[[4-(trifluoromethyl) thiazol-2-yl]methyl]-2-azaspiro[3.3]heptan-2-yl]methanone;
(3S)-1-[6-[4-(Trifluoromethyl)pyrazol-1-yl]methyl]-2-azaspiro[3.3]heptane-2-carbonyl]pyrrolidine-3-carboxamide;
(3S)-1-[6-[4-(Trifluoromethyl) imidazol-1-yl]methyl]-2-azaspiro[3.3]heptane-2-carbonyl]pyrrolidine-3-carboxamide;
(3S)-1-[6-[3-(Trifluoromethyl)-1,2,4-triazol-1-yl]methyl]-2-azaspiro[3.3]heptane-2-carbonyl]pyrrolidine-3-carboxamide;
(3S)-1-[6-[[4-(Trifluoromethyl) oxazol-2-yl]methyl]-2-azaspiro[3.3]heptane-2-carbonyl]pyrrolidine-3-carboxamide;
(3S)-1-[6-[3-(Trifluoromethyl)-1,2,4-thiadiazol-5-yl]methyl]-2-azaspiro[3.3]heptane-2-carbonyl]pyrrolidine-3-carboxamide;
(3S)-1-[6-[4-(Trifluoromethyl) thiazol-2-yl]methyl]-2-azaspiro[3.3]heptane-2-carbonyl]pyrrolidine-3-carboxamide;
[6-[(5-Fluoro-3-pyridyl)methyl]-2-azaspiro[3.3]heptan-2-yl]-[(3S)-3-(4H-1,2,4-triazol-3-yl)pyrrolidin-1-yl]methanone;
[6-[(2-Fluoro-4-methylsulfonyl-phenyl)methyl]-2-azaspiro[3.3]heptan-2-yl]-[(3S)-3-(4H-1,2,4-triazol-3-yl)pyrrolidin-1-yl]methanone;
[6-([1,2,4]Triazolo[1,5-a]pyridin-7-ylmethyl)-2-azaspiro[3.3]heptan-2-yl]-[(3S)-3-(1H-1,2,4-triazol-5-yl)pyrrolidin-1-yl]methanone;
(3S)-1-[6-([1,2,4]Triazolo[1,5-a]pyridin-6-ylmethyl)-2-azaspiro[3.3]heptane-2-carbonyl]pyrrolidine-3-carboxamide;
[6-(1H-Pyrazolo[4,3-b]pyridin-5-ylmethyl)-2-azaspiro[3.3]heptan-2-yl]-[(3S)-3-(1H-1,2,4-triazol-5-yl)pyrrolidin-1-yl]methanone;
[(3S)-3-(1H-1,2,4-Triazol-5-yl)pyrrolidin-1-yl]-[6-[1-(trifluoromethyl)pyrazol-4-yl]methyl]-2-azaspiro[3.3]heptan-2-yl]methanone;
3-[[2-[(3S)-3-(1H-1,2,4-Triazol-5-yl)pyrrolidine-1-carbonyl]-2-azaspiro[3.3]heptan-6-yl]methyl]-5-(trifluoromethyl)benzonitrile;
4-[[2-[(3S)-3-(1H-1,2,4-Triazol-5-yl)pyrrolidine-1-carbonyl]-2-azaspiro[3.3]heptan-6-yl]methyl]-2-(trifluoromethyl)benzonitrile;
[(3S)-3-(1H-1,2,4-Triazol-5-yl)pyrrolidin-1-yl]-[6-[6-(trifluoromethoxy)-3-pyridyl]methyl]-2-azaspiro[3.3]heptan-2-yl]methanone;
[(3S)-3-(1H-1,2,4-Triazol-5-yl)pyrrolidin-1-yl]-[6-[[3-(trifluoromethylsulfonyl)phenyl]methyl]-2-azaspiro[3.3]heptan-2-yl]methanone;
[6-[(4-Cyclopropylsulfonylphenyl)methyl]-2-azaspiro[3.3]heptan-2-yl]-[(3S)-3-(1H-1,2,4-triazol-5-yl)pyrrolidin-1-yl]methanone;
[(3S)-3-(1H-1,2,4-Triazol-5-yl)pyrrolidin-1-yl]-[3-[3-[[6-(trifluoromethyl)-3-pyridyl]methyl]-1-bicyclo[1.1.1]pentanyl]azetidin-1-yl]methanone;
[6-[[2-Fluoro-4-(trifluoromethyl)phenyl]methyl]-2-azaspiro[3.3]heptan-2-yl]-[3-(1H-1,2,4-triazol-5-yl)azetidin-1-yl]methanone;
[6-[(2,4-Difluorophenyl)methyl]-2-azaspiro[3.3]heptan-2-yl]-[3-(1H-1,2,4-triazol-5-yl)azetidin-1-yl]methanone;
[6-[4-Fluoro-2-(trifluoromethyl)phenyl]methyl]-2-azaspiro[3.3]heptan-2-yl]-[3-(1H-1,2,4-triazol-5-yl)azetidin-1-yl]methanone;
[6-[[3-Fluoro-5-(trifluoromethyl)phenyl]methyl]-2-azaspiro[3.3]heptan-2-yl]-[3-(1H-1,2,4-triazol-5-yl)azetidin-1-yl]methanone;
[6-[(3-Chloro-5-fluoro-2-pyridyl)methyl]-2-azaspiro[3.3]heptan-2-yl]-[3-(1H-1,2,4-triazol-5-yl)azetidin-1-yl]methanone;
[3-(1H-1,2,4-Triazol-5-yl)azetidin-1-yl]-[6-[5-(trifluoromethoxy)-2-pyridyl]methyl]-2-azaspiro[3.3]heptan-2-yl]methanone;
[6-[[3-Fluoro-5-(trifluoromethyl)-2-pyridyl]methyl]-2-azaspiro[3.3]heptan-2-yl]-[3-(1H-1,2,4-triazol-5-yl)azetidin-1-yl]methanone;
[3-(1H-1,2,4-Triazol-5-yl)azetidin-1-yl]-[7-[5-(trifluoromethyl)-2-pyridyl]methyl]-2,7-diazaspiro[3.4]octan-2-yl]methanone;
[7-[(5-Chloro-2-pyridyl)methyl]-2-azaspiro[3.5]nonan-2-yl]-[3-(1H-1,2,4-triazol-5-yl)azetidin-1-yl]methanone;
[7-[6-(Difluoromethoxy)-3-pyridyl]methyl]-2-azaspiro[3.5]nonan-2-yl]-[3-(1H-1,2,4-triazol-5-yl)azetidin-1-yl]methanone;
[3-(1H-1,2,4-Triazol-5-yl)azetidin-1-yl]-[3-[4-[1-(trifluoromethyl)cyclopropyl]phenyl]azetidin-1-yl]methanone;
[3-(1H-1,2,4-Triazol-5-yl)azetidin-1-yl]-[3-[4-[3-(trifluoromethyl)azetidin-1-yl]phenyl]azetidin-1-yl]methanone;
[3-[4-(4-Chloro-2-methylsulfonyl-phenyl)phenyl]azetidin-1-yl]-[3-(1H-1,2,4-triazol-5-yl)azetidin-1-yl]methanone;
[3-[4-[5-[(1-Methylcyclopropyl)methyl]-4H-1,2,4-triazol-3-yl]phenyl]azetidin-1-yl]-[(3S)-3-(1H-1,2,4-triazol-5-yl)pyrrolidin-1-yl]methanon;
[3-[4-(4-Chloro-2-methylsulfonyl-phenyl)phenyl]azetidin-1-yl]-[3-(5-cyclopropyl-1H-1,2,4-triazol-3-yl)pyrrolidin-1-yl]methanone;
(3S)-1-[3-[4-[5-Methyl-3-(trifluoromethyl)pyrazol-1-yl]phenyl]azetidine-1-carbonyl]pyrrolidine-3-carboxamide;
[4-[(2-Chloro-4-methylsulfonyl-phenyl)methoxy]-1-piperidyl]-[(3S)-3-(1H-1,2,4-triazol-5-yl)pyrrolidin-1-yl]methanone;
(3S)-1-[4-[(2-Chloro-4-methylsulfonyl-phenyl)methoxy]piperidine-1-carbonyl]pyrrolidine-3-carboxamide;
[4-[[4-Methylsulfonyl-3-(trifluoromethyl) phenoxy]methyl]-1-piperidyl]-[(3S)-3-(1H-1,2,4-triazol-5-yl)pyrrolidin-1-yl]methanone;
(3S)-1-[4-[[4-Methylsulfonyl-3-(trifluoromethyl) phenoxy]methyl]piperidine-1-carbonyl]pyrrolidine-3-carboxamide;
[3-[3-(5-Cyclopropyl-3-methyl-pyrazol-1-yl)-1-bicyclo[1.1.1]pentanyl]azetidin-1-yl]-[(3S)-3-(1H-1,2,4-triazol-5-yl)pyrrolidin-1-yl]methanone;
[3-[3-(3,5-Dimethylpyrazol-1-yl)-1-bicyclo[1.1.1]pentanyl]azetidin-1-yl]-[(3S)-3-(1H-1,2,4-triazol-5-yl)pyrrolidin-1-yl]methanone;
[4-[[2-Fluoro-4-(trifluoromethoxy)phenyl]methoxy]-1-piperidyl]-[(3S)-3-(1H-1,2,4-triazol-5-yl)pyrrolidin-1-yl]methanone;
[4-[[2-Fluoro-4-(trifluoromethyl)phenyl]methoxy]-1-piperidyl]-[(3S)-3-(1H-1,2,4-triazol-5-yl)pyrrolidin-1-yl]methanone;
(3S)-1-[4-[[2-Fluoro-4-(trifluoromethoxy)phenyl]methoxy]piperidine-1-carbonyl]pyrrolidine-3-carboxamide;
(3S)-1-[4-[2-Fluoro-4-(trifluoromethyl)phenyl]methoxy]piperidine-1-carbonyl]pyrrolidine-3-carboxamide;
Methyl 5-[2-[(3S)-3-(1H-1,2,4-triazol-5-yl)pyrrolidine-1-carbonyl]-2-azaspiro[3.3]heptan-6-yl]methyl]-2-(trifluoromethoxy)benzoate;
[4-[[2-Methylsulfonyl-4-(trifluoromethyl) phenoxy]methyl]-1-piperidyl]-[(3S)-3-(1H-1,2,4-triazol-5-yl)pyrrolidin-1-yl]methanone;
[3-[4-(4-Chloro-2-methylsulfonyl-phenyl)phenyl]azetidin-1-yl]-[4-(5-cyclopropyl-1H-1,2,4-triazol-3-yl) piperazin-1-yl]methanone;
[(3S)-3-(1H-1,2,4-Triazol-5-yl)pyrrolidin-1-yl]-[6-[5-(trifluoromethylsulfonyl)-2-pyridyl]methyl]-2-azaspiro[3.3]heptan-2-yl]methanone;
[4-(5-Chlorooxazolo[4,5-b]pyridin-2-yl) piperazin-1-yl]-[3-[[2-fluoro-4-(trifluoromethoxy)phenyl]methoxy]azetidin-1-yl]methanone;
4-[2-[(3S)-3-(1H-1,2,4-Triazol-5-yl)pyrrolidine-1-carbonyl]-2-azaspiro[3.3]heptan-6-yl]methyl]benzenesulfonamide;
[(3S)-3-(1H-1,2,4-Triazol-5-yl)pyrrolidin-1-yl]-[6-[[5-[3-(trifluoromethyl)azetidin-1-yl]pyrazin-2-yl]methyl]-2-azaspiro[3.3]heptan-2-yl]methanone;
[(3S)-3-(4H-1,2,4-Triazol-3-yl)pyrrolidin-1-yl]-[6-[5-[1-(trifluoromethyl)cyclopropyl]methylamino]pyrazin-2-yl]methyl]-2-azaspiro[3.3]heptan-2-yl]methanone;
[(3S)-3-(4H-1,2,4-Triazol-3-yl)pyrrolidin-1-yl]-[6-[[4-[3-(trifluoromethyl)azetidin-1-yl]sulfonylphenyl]methyl]-2-azaspiro[3.3]heptan-2-yl]methanone;
[(3S)-3-(1H-1,2,4-Triazol-5-yl)pyrrolidin-1-yl]-[(6S)-6-[[4-(trifluoromethylsulfonyl)phenyl]methyl]-2-azaspiro[3.4]octan-2-yl]methanone;
[(3S)-3-(1H-1,2,4-Triazol-5-yl)pyrrolidin-1-yl]-[(6R)-6-[[4-(trifluoromethylsulfonyl)phenyl]methyl]-2-azaspiro[3.4]octan-2-yl]methanone;
[(3S)-3-(1H-1,2,4-Triazol-5-yl)pyrrolidin-1-yl]-[(6S)-6-[[3-(trifluoromethylsulfonyl)phenyl]methyl]-2-azaspiro[3.4]octan-2-yl]methanone;
[(3S)-3-(1H-1,2,4-Triazol-5-yl)pyrrolidin-1-yl]-[(6R)-6-[3-(trifluoromethylsulfonyl)phenyl]methyl]-2-azaspiro[3.4]octan-2-yl]methanone;
[6-[1-Methyl-3-(trifluoromethyl)pyrazol-4-yl]methyl]-2-azaspiro[3.3]heptan-2-yl]-[(3S)-3-(1H-1,2,4-triazol-5-yl)pyrrolidin-1-yl]methanone;
[6-[[2-Methyl-5-(trifluoromethyl)pyrazol-3-yl]methyl]-2-azaspiro[3.3]heptan-2-yl]-[(3S)-3-(1H-1,2,4-triazol-5-yl)pyrrolidin-1-yl]methanone;
[6-[[1-Methyl-5-(trifluoromethyl)pyrazol-4-yl]methyl]-2-azaspiro[3.3]heptan-2-yl]-[(3S)-3-(1H-1,2,4-triazol-5-yl)pyrrolidin-1-yl]methanone;
[6-[[2-Methyl-4-(trifluoromethyl)pyrazol-3-yl]methyl]-2-azaspiro[3.3]heptan-2-yl]-[(3S)-3-(1H-1,2,4-triazol-5-yl)pyrrolidin-1-yl]methanone;
[(3S)-3-(1H-1,2,4-Triazol-5-yl)pyrrolidin-1-yl]-[6-[[1-(2,2,2-trifluoroethyl)pyrazol-4-yl]methyl]-2-azaspiro[3.3]heptan-2-yl]methanone;
4-[2-[(3S)-3-(1H-1,2,4-Triazol-5-yl)pyrrolidine-1-carbonyl]-2-azaspiro[3.3]heptan-6-yl]methyl]-2-(trifluoromethoxy)benzamide;
[(3S)-3-(4H-1,2,4-Triazol-3-yl)pyrrolidin-1-yl]-[3-[5-[1-(trifluoromethyl)cyclopropyl]-2-pyridyl]azetidin-1-yl]methanone;
[(3S)-3-(4H-1,2,4-Triazol-3-yl)pyrrolidin-1-yl]-[3-[[6-(trifluoromethyl)-3-pyridyl]methoxymethyl]azetidin-1-yl]methanone;
[3-[4-(1-Methylsulfonylcyclopropyl)phenyl]azetidin-1-yl]-[(3S)-3-(1H-1,2,4-triazol-5-yl)pyrrolidin-1-yl]methanone;
[4-[(3-Chloro-5-methylsulfonyl-phenoxy)methyl]-1-piperidyl]-[(3S)-3-(1H-1,2,4-triazol-5-yl)pyrrolidin-1-yl]methanone;
5-[2-[(3S)-3-(1H-1,2,4-Triazol-5-yl)pyrrolidine-1-carbonyl]-2-azaspiro[3.3]heptan-6-yl]methyl]-2-(trifluoromethoxy)benzamide;
[rac-(3S)-3-(1H-1,2,4-Triazol-5-yl)pyrrolidin-1-yl]-[3-[4-(trifluoromethylsulfonimidoyl)phenyl]azetidin-1-yl]methanone;
[(3S)-3-(1H-1,2,4-Triazol-5-yl)pyrrolidin-1-yl]-[3-[3-[[5-(trifluoromethyl)pyrazin-2-yl]amino]-1-bicyclo[1.1.1]pentanyl]azetidin-1-yl]methanone;
Methyl 4-[[2-[(3S)-3-(1H-1,2,4-triazol-5-yl)pyrrolidine-1-carbonyl]-2-azaspiro[3.3]heptan-6-yl]methyl]-2-(trifluoromethoxy)benzoate;
[4-[[2-Methylsulfonyl-4-(trifluoromethoxy) phenoxy]methyl]-1-piperidyl]-[(3S)-3-(4H-1,2,4-triazol-3-yl)pyrrolidin-1-yl]methanone;
[(3S)-3-(4H-1,2,4-Triazol-3-yl)pyrrolidin-1-yl]-[4-[[6-(trifluoromethyl)-3-pyridyl]methoxymethyl]-1-piperidyl]methanone;
[(3S)-3-(4H-1,2,4-Triazol-3-yl)pyrrolidin-1-yl]-[4-[6-(trifluoromethyl)-3-pyridyl]oxymethyl]-1-piperidyl]methanone;
[(3S)-3-(4H-1,2,4-Triazol-3-yl)pyrrolidin-1-yl]-[4-[6-(trifluoromethyl)-3-pyridyl]methoxy]-1-piperidyl]methanone;
[4-[4-Fluoro-3-(trifluoromethyl) phenoxy]methyl]-1-piperidyl]-[(3S)-3-(4H-1,2,4-triazol-3-yl)pyrrolidin-1-yl]methanone;
[4-[[3-Fluoro-5-(trifluoromethyl) phenoxy]methyl]-1-piperidyl]-[(3S)-3-(4H-1,2,4-triazol-3-yl)pyrrolidin-1-yl]methanone;
[3-(3-Isobutyl-1H-pyrazol-5-yl)pyrrolidin-1-yl]-[6-[5-(trifluoromethyl)pyrazin-2-yl]methyl]-2-azaspiro[3.3]heptan-2-yl]methanone;
[(3S)-3-(4H-1,2,4-Triazol-3-yl)pyrrolidin-1-yl]-[6-[5-(trifluoromethylsulfonyl)-3-pyridyl]methyl]-2-azaspiro[3.3]heptan-2-yl]methanone;
[6-[[3-Fluoro-5-(trifluoromethylsulfonyl)phenyl]methyl]-2-azaspiro[3.3]heptan-2-yl]-[(3S)-3-(4H-1,2,4-triazol-3-yl)pyrrolidin-1-yl]methanone;
N′-[2-[3-[1-[(3S)-3-(4H-1,2,4-Triazol-3-yl)pyrrolidine-1-carbonyl]azetidin-3-yl]-1-bicyclo[1.1.1]pentanyl]acetyl]cyclopropanecarbohydrazide;
[4-[2-Methylsulfonyl-4-(trifluoromethoxy)phenyl]methoxy]-1-piperidyl]-[(3S)-3-(1H-1,2,4-triazol-5-yl)pyrrolidin-1-yl]methanone;
(3S)-1-[4-[2-Methylsulfonyl-4-(trifluoromethoxy)phenyl]methoxy]piperidine-1-carbonyl]pyrrolidine-3-carboxamide;
(3S)-1-[3-[3-(4-Chloro-2-methylsulfonyl-phenyl)-1-bicyclo[1.1.1]pentanyl]azetidine-1-carbonyl]pyrrolidine-3-carboxamide;
2-Methoxy-4-[[1-[(3S)-3-(1H-1,2,4-triazol-5-yl)pyrrolidine-1-carbonyl]-4-piperidyl]oxymethyl]benzonitrile;
(3S)-1-[2-(4-Chloro-2-methylsulfonyl-phenyl)-6-azaspiro[3.4]octane-6-carbonyl]pyrrolidine-3-carboxamide;
2-Methoxy-4-[[1-[(3S)-3-(1H-1,2,4-triazol-5-yl)pyrrolidine-1-carbonyl]-4-piperidyl]oxymethyl]benzamide;
(3S)-1-[3-[3-[2-Methylsulfonyl-4-(trifluoromethyl)phenyl]-1-bicyclo[1.1.1]pentanyl]azetidine-1-carbonyl]pyrrolidine-3-carboxamide;
(3S)-1-[2-[(4-Chloro-2-methylsulfonyl-phenyl)methyl]-6-azaspiro[3.4]octane-6-carbonyl]pyrrolidine-3-carboxamide;
[(3S)-3-(1H-1,2,4-Triazol-5-yl)pyrrolidin-1-yl]-[6-[3-(trifluoromethoxy)phenyl]sulfonyl-2-azaspiro[3.3]heptan-2-yl]methanone;
[(3S)-3-(1H-1,2,4-Triazol-5-yl)pyrrolidin-1-yl]-[7-[3-(trifluoromethyl)phenyl]sulfonyl-2-azaspiro[3.5]nonan-2-yl]methanone;
[(3S)-3-(1H-1,2,4-Triazol-5-yl)pyrrolidin-1-yl]-[7-[4-(trifluoromethyl)phenyl]sulfonyl-2-azaspiro[3.5]nonan-2-yl]methanone;
[3-[3-(4-Fluorophenyl)-1-bicyclo[1.1.1]pentanyl]azetidin-1-yl]-[(3S)-3-(1H-1,2,4-triazol-5-yl)pyrrolidin-1-yl]methanone;
[3-[3-(4-Methylsulfonylphenyl)-1-bicyclo[1.1.1]pentanyl]azetidin-1-yl]-[(3S)-3-(1H-1,2,4-triazol-5-yl)pyrrolidin-1-yl]methanone;
[(3S)-3-(1H-1,2,4-Triazol-5-yl)pyrrolidin-1-yl]-[6-[[4-(trifluoromethylsulfonimidoyl)phenyl]methyl]-2-azaspiro[3.3]heptan-2-yl]methanone;
[(3S)-3-(1H-1,2,4-Triazol-5-yl)pyrrolidin-1-yl]-[6-[[3-(trifluoromethylsulfonimidoyl)phenyl]methyl]-2-azaspiro[3.3]heptan-2-yl]methanone;
[6-[[3-(Methylsulfonimidoyl)-5-(trifluoromethyl)phenyl]methyl]-2-azaspiro[3.3]heptan-2-yl]-[rac-(3S)-3-(1H-1,2,4-triazol-5-yl)pyrrolidin-1-yl]methanone;
[3-[[2-Fluoro-4-(trifluoromethylsulfonimidoyl)phenyl]methoxy]azetidin-1-yl]-[rac-(3S)-3-(1H-1,2,4-triazol-5-yl)pyrrolidin-1-yl]methanone;
[(3S)-3-(1H-1,2,4-Triazol-5-yl)pyrrolidin-1-yl]-[4-[[5-(trifluoromethyl)pyrazin-2-yl]amino]cuban-1-yl]methanone;
[(3S)-3-(1H-1,2,4-Triazol-5-yl)pyrrolidin-1-yl]-[6-[[4-(trifluoromethylsulfonyl)phenyl]methyl]-2-azaspiro[3.3]heptan-2-yl]methanone;
[rac-(3S)-3-(1H-1,2,4-Triazol-5-yl)pyrrolidin-1-yl]-[6-[[3-(trifluoromethoxy)phenyl]sulfonimidoyl]-2-azaspiro[3.3]heptan-2-yl]methanone;
[6-[3-Fluoro-5-(trifluoromethylsulfonimidoyl)phenyl]methyl]-2-azaspiro[3.3]heptan-2-yl]-[rac-(3S)-3-(1H-1,2,4-triazol-5-yl)pyrrolidin-1-yl]methanone;
[rac-(3S)-3-(1H-1,2,4-Triazol-5-yl)pyrrolidin-1-yl]-[7-[4-(trifluoromethyl)phenyl]sulfonimidoyl]-2-azaspiro[3.5]nonan-2-yl]methanone;
[rac-(3S)-3-(1H-1,2,4-Triazol-5-yl)pyrrolidin-1-yl]-[7-[[3-(trifluoromethyl)phenyl]sulfonimidoyl]-2-azaspiro[3.5]nonan-2-yl]methanone;
[3-[4-[2-(Methylsulfonimidoyl)phenyl]phenyl]azetidin-1-yl]-[rac-(3S)-3-(1H-1,2,4-triazol-5-yl)pyrrolidin-1-yl]methanone;
[3-[(8R)-8-Methyl-8-oxo-8lambda6-thia-9-azatricyclo[8.4.0.02,7]tetradeca-1(10),2 (7),3,5,8,11,13-heptaen-12-yl]azetidin-1-yl]-[(3S)-3-(1H-1,2,4-triazol-5-yl)pyrrolidin-1-yl]methanone;
[3-[(8S)-8-Methyl-8-oxo-8lambda6-thia-9-azatricyclo[8.4.0.02,7]tetradeca-1(10),2 (7), 3,5,8,11,13-heptaen-12-yl]azetidin-1-yl]-[(3S)-3-(1H-1,2,4-triazol-5-yl)pyrrolidin-1-yl]methanone;
[3-[3-(4-Fluoro-2-methylsulfonyl-phenyl)-1-bicyclo[1.1.1]pentanyl]azetidin-1-yl]-[(3S)-3-(1H-1,2,4-triazol-5-yl)pyrrolidin-1-yl]methanone;
[(3S)-3-(1H-1,2,4-Triazol-5-yl)pyrrolidin-1-yl]-[3-[3-[3-(trifluoromethoxy)phenyl]-1-bicyclo[1.1.1]pentanyl]azetidin-1-yl]methanone;
[(3S)-3-(4H-1,2,4-Triazol-3-yl)pyrrolidin-1-yl]-[4-[[5-(trifluoromethyl)-2-pyridyl]methyl]piperazin-1-yl]methanone;
[4-[(2-Methyloxazol-4-yl)methyl]piperazin-1-yl]-[(3S)-3-(1H-1,2,4-triazol-5-yl)pyrrolidin-1-yl]methanone;
[4-[(4-Methylthiazol-5-yl)methyl]piperazin-1-yl]-[(3S)-3-(1H-1,2,4-triazol-5-yl)pyrrolidin-1-yl]methanone;
[4-[(5-Chloro-1,3-dimethyl-pyrazol-4-yl)methyl]piperazin-1-yl]-[(3S)-3-(1H-1,2,4-triazol-5-yl)pyrrolidin-1-yl]methanone;
4-[2-[(3S)-3-(1H-1,2,4-Triazol-5-yl)pyrrolidine-1-carbonyl]-2-azaspiro[3.3]heptan-6-yl]oxy]-2-(trifluoromethyl)benzonitrile;
5-Fluoro-2-[3-[1-[(3S)-3-(1H-1,2,4-triazol-5-yl)pyrrolidine-1-carbonyl]azetidin-3-yl]-1-bicyclo[1.1.1]pentanyl]benzonitrile;
[3-[3-(3-Methyl-1H-pyrazol-5-yl)-1-bicyclo[1.1.1]pentanyl]azetidin-1-yl]-[(3S)-3-(1H-1,2,4-triazol-5-yl)pyrrolidin-1-yl]methanone;
[6-[1-Methyl-4-(trifluoromethyl)pyrazol-3-yl]methyl]-2-azaspiro[3.3]heptan-2-yl]-[(3S)-3-(4H-1,2,4-triazol-3-yl)pyrrolidin-1-yl]methanone;
[(3S)-3-(1H-1,2,4-Triazol-5-yl)pyrrolidin-1-yl]-[3-[3-[4-(trifluoromethylsulfonyl)phenyl]-1-bicyclo[1.1.1]pentanyl]azetidin-1-yl]methanone;
[3-[3-(4-Chloro-2-methylsulfonyl-phenyl)-1-bicyclo[1.1.1]pentanyl]azetidin-1-yl]-[(3S)-3-(1H-1,2,4-triazol-5-yl)pyrrolidin-1-yl]methanone;
[6-[(5-Chloro-1,3-dimethyl-pyrazol-4-yl)methyl]-2,6-diazaspiro[3.3]heptan-2-yl]-[(3S)-3-(1H-1,2,4-triazol-5-yl)pyrrolidin-1-yl]methanone;
5-[2-[(3S)-3-(1H-1,2,4-Triazol-5-yl)pyrrolidine-1-carbonyl]-2-azaspiro[3.3]heptan-6-yl]oxy]-2-(trifluoromethyl)benzonitrile;
[6-[(5-Fluoro-2-pyridyl)oxy]-2-azaspiro[3.3]heptan-2-yl]-[(3S)-3-(1H-1,2,4-triazol-5-yl)pyrrolidin-1-yl]methanone;
[6-[1-Methyl-3-(trifluoromethyl)pyrazol-4-yl]oxy-2-azaspiro[3.3]heptan-2-yl]-[(3S)-3-(1H-1,2,4-triazol-5-yl)pyrrolidin-1-yl]methanone;
[(3S)-3-(1H-1,2,4-Triazol-5-yl)pyrrolidin-1-yl]-[3-[[5-(trifluoromethyl)pyrazin-2-yl]amino]-1-bicyclo[1.1.1]pentanyl]methanone;
(3S)-3-(1H-1,2,4-triazol-5-yl)pyrrolidin-1-yl]-[6-[4-(trifluoromethylsulfonimidoyl) phenoxy]-2-azaspiro[3.3]heptan-2-yl]methanone;
[(3S)-3-(1H-triazol-5-yl)pyrrolidin-1-yl]-[6-[[3-(trifluoromethylsulfonimidoyl)phenyl]methyl]-2-azaspiro[3.3]heptan-2-yl]methanone;
[(3S)-3-(1H-triazol-5-yl)pyrrolidin-1-yl]-[6-[[3-(trifluoromethylsulfonimidoyl)phenyl]methyl]-2-azaspiro[3.3]heptan-2-yl]methanone;
[(3S)-3-(1H-triazol-5-yl)pyrrolidin-1-yl]-[6-[[3-(trifluoromethylsulfonyl)phenyl]methyl]-2-azaspiro[3.3]heptan-2-yl]methanone;
5-[2-[(3S)-3-(1H-triazol-5-yl)pyrrolidine-1-carbonyl]-2-azaspiro[3.3]heptan-6-yl]methyl]-2-(trifluoromethyl)benzonitrile;
(3S)-1-[6-[1-methyl-5-(trifluoromethyl)pyrazol-3-yl]methyl]-2-azaspiro[3.3]heptane-2-carbonyl]pyrrolidine-3-carboxamide;
(3S)-1-[6-[1-methyl-5-(trifluoromethyl)pyrazol-4-yl]methyl]-2-azaspiro[3.3]heptane-2-carbonyl]pyrrolidine-3-carboxamide;
(3S)-1-[6-[3-(trifluoromethylsulfonyl)phenyl]methyl]-2-azaspiro[3.3]heptane-2-carbonyl]pyrrolidine-3-carboxamide;
(3S)-1-[6-[[4-cyano-3-(trifluoromethyl)phenyl]methyl]-2-azaspiro[3.3]heptane-2-carbonyl]pyrrolidine-3-carboxamide;
(3S)-1-[6-[6-(trifluoromethoxy)-3-pyridyl]methyl]-2-azaspiro[3.3]heptane-2-carbonyl]pyrrolidine-3-carboxamide;
5-[2-[(3S)-3-(1H-1,2,4-triazol-5-yl)pyrrolidine-1-carbonyl]-2-azaspiro[3.3]heptan-6-yl]methyl]-2-(trifluoromethyl)benzonitrile;
[(3S)-3-(1H-triazol-5-yl)pyrrolidin-1-yl]-[6-[4-(trifluoromethylsulfonimidoyl)phenyl]methyl]-2-azaspiro[3.3]heptan-2-yl]methanone;
[(3S)-3-(1H-triazol-5-yl)pyrrolidin-1-yl]-[6-[[4-(trifluoromethylsulfonimidoyl)phenyl]methyl]-2-azaspiro[3.3]heptan-2-yl]methanone;
rac-(3S)-1-[6-[[2-fluoro-4-(trifluoromethylsulfonimidoyl)phenyl]methyl]-2-azaspiro[3.3]heptane-2-carbonyl]pyrrolidine-3-carboxamide;
[6-[[2-fluoro-4-(trifluoromethylsulfonimidoyl)phenyl]methyl]-2-azaspiro[3.3]heptan-2-yl]-[rac-(3S)-3-(1H-triazol-4-yl)pyrrolidin-1-yl]methanone;
[(3S)-3-(1H-triazol-4-yl)pyrrolidin-1-yl]-[6-[4-(trifluoromethylsulfonyl)phenyl]methyl]-2-azaspiro[3.3]heptan-2-yl]methanone;
[6-[[3-fluoro-5-(trifluoromethylsulfonyl)phenyl]methyl]-2-azaspiro[3.3]heptan-2-yl]-[(3S)-3-(1H-triazol-4-yl)pyrrolidin-1-yl]methanone;
[(3S)-3-(1H-triazol-4-yl)pyrrolidin-1-yl]-[6-[[5-(trifluoromethyl)-1H-pyrazol-3-yl]methyl]-2-azaspiro[3.3]heptan-2-yl]methanone; and
[6-[[2-fluoro-4-(trifluoromethylsulfonimidoyl)phenyl]methyl]-2-azaspiro[3.3]heptan-2-yl]-[rac-(3S)-3-(1H-1,2,4-triazol-3-yl)pyrrolidin-1-yl]methanone.

13. A compound of formula (I) according to claim 12, or a pharmaceutically acceptable salt thereof, selected from:
[3-[[2-Fluoro-4-(trifluoromethyl)phenyl]methoxy]azetidin-1-yl]-[3-(1H-1,2,4-triazol-5-yl)pyrrolidin-1-yl]methanone;
[3-[[2-Fluoro-4-(trifluoromethyl)phenyl]methoxy]azetidin-1-yl]-[(3S)-3-(1H-1,2,4-triazol-5-yl)pyrrolidin-1-yl]methanone;
1-[3-[[2-Fluoro-4-(trifluoromethyl)phenyl]methoxy]azetidine-1-carbonyl]pyrrolidine-3-carboxamide;
[3-[2-Fluoro-4-(trifluoromethyl)phenyl]methoxy]azetidin-1-yl]-[3-(1H-triazol-5-yl)pyrrolidin-1-yl]methanone;
(+)- or (−)-[2-(2-Chloro-4-fluoro-phenoxy)-7-azaspiro[3.5]nonan-7-yl]-[(3R)-3-(1H-1,2,4-triazol-5-yl)pyrrolidin-1-yl]methanone;
[3-[[2-Fluoro-4-(trifluoromethyl)phenyl]methoxy]azetidin-1-yl]-[3-(1H-imidazol-5-yl)pyrrolidin-1-yl]methanone;
[3-[[2-Fluoro-4-(trifluoromethyl)phenyl]methoxy]azetidin-1-yl]-[(3S)-3-(1H-triazol-5-yl)pyrrolidin-1-yl]methanone;
[3-[[2-Fluoro-4-(trifluoromethyl)phenyl]methoxy]azetidin-1-yl]-[(3R)-3-(1H-triazol-5-yl)pyrrolidin-1-yl]methanone;
[3-[[3-Chloro-4-(trifluoromethyl)phenyl]methoxy]azetidin-1-yl]-[3-(1H-1,2,4-triazol-5-yl)pyrrolidin-1-yl]methanone;
[3-[[2-Chloro-4-(trifluoromethyl)phenyl]methoxy]azetidin-1-yl]-[3-(1H-1,2,4-triazol-5-yl)pyrrolidin-1-yl]methanone;
(−)- or (+)-[3-[2,4-bis(Trifluoromethyl)phenyl]methoxy]azetidin-1-yl]-[3-(1H-1,2,4-triazol-5-yl)pyrrolidin-1-yl]methanone;
(+)- or (−)-1-[3-[4-(2,4-Difluorophenyl)phenyl]azetidine-1-carbonyl]pyrrolidine-3-carboxamide;
(+)- or (−)-[3-(1H-Triazol-5-yl)pyrrolidin-1-yl]-[3-[4-[1-(trifluoromethyl)cyclopropyl]phenyl]azetidin-1-yl]methanone;
(−)- or (+)-[3-(1H-Triazol-5-yl)pyrrolidin-1-yl]-[3-[4-[1-(trifluoromethyl)cyclopropyl]phenyl]azetidin-1-yl]methanone; and
4-[1-[3-[4-[1-(Trifluoromethyl)cyclopropyl]phenyl]azetidine-1-carbonyl]pyrrolidin-3-yl]oxazolidin-2-one.

14. A process of manufacturing a compound of formula (IA) or (IB) according to any one of claims 1 to 13, comprising:
(a) reacting an amine of formula 2, wherein p, q, and R⁴ are as defined in any one of claims 1 to 13, 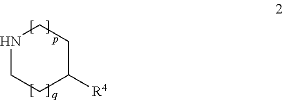
with a carboxylic acid 3a, wherein L, A, B, and R¹ to R³ are as defined in any one of claims 1 to 13 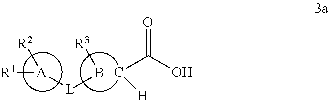
in the presence of a coupling reagent selected from CDI, DCC, HATU, HBTU, HOBT, TBTU, T3P and Mukaiyama reagent, and optionally in the presence of a base selected from TEA, DIPEA (Huenig's base) and DMAP, to form said compound of formula (IB), wherein A, B, L, p, q, and R¹ to R⁴ are as defined in any one of claims 1 to 13 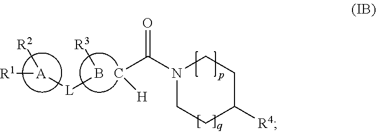
or
(b) reacting an amine of formula 2, wherein p, q, and R⁴ are as defined in any one of claims 1 to 13, 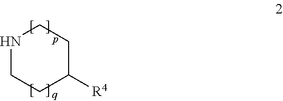
with a carboxylic acid chloride 3b, wherein L, A, B, and R¹ to R³ are as defined in any one of claims 1 to 13 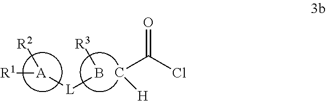
in the presence of a base selected from TEA, Huenig's base, pyridine, DMAP and lithium bis(trimethylsilyl)amide, to form said compound of formula (IB), wherein A, B, L, p, q, and R¹ to R⁴ are as defined in any one of claims 1 to 13; or
(c) reacting a first amine of formula 1, wherein A, B, L, and R¹ to R³ are as defined in any one of claims 1 to 13, 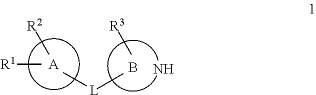
with a second amine 2, wherein X, Y, and Z are as defined in any one of claims 1 to 13 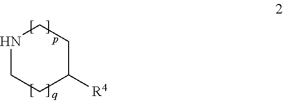
in the presence of sodium bicarbonate, and a urea forming reagent selected from bis(trichloromethyl) carbonate, phosgene, trichloromethyl chloroformate, (4-nitrophenyl) carbonate and 1,1′-carbonyldiimidazole,
to form said compound of formula (IA), wherein A, B, L, p, q, and R¹ to R⁴ are as defined in any one of claims 1 to 13

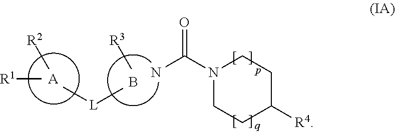

15. A compound of formula (I) according to any one of claims 1 to 13, or a pharmaceutically acceptable salt thereof, when manufactured according to the process of claim 14.

16. A compound of formula (I) according to any one of claims 1 to 13 and 15, or a pharmaceutically acceptable salt thereof, for use as therapeutically active substance.

17. A pharmaceutical composition comprising a compound of formula (I) according to any one of claims 1 to 13 and 15, or a pharmaceutically acceptable salt thereof, and a therapeutically inert carrier.

18. The use of a compound of formula (I) according to any one of claims 1 to 13 and 15, or a pharmaceutically acceptable salt thereof, or of a pharmaceutical composition according to claim 17 for the treatment or prophylaxis of neuroinflammation, neurodegenerative diseases, pain, cancer, mental disorders and/or inflammatory bowel disease in a mammal.

19. The use of a compound of formula (I) according to any one of claims 1 to 13 and 15, or a pharmaceutically acceptable salt thereof, or of a pharmaceutical composition according to claim 17 for the treatment or prophylaxis of multiple sclerosis, Alzheimer's disease, Parkinson's disease, amyotrophic lateral sclerosis, traumatic brain injury, neurotoxicity, stroke, epilepsy, anxiety, migraine, depression, hepatocellular carcinoma, colon carcinogenesis, ovarian cancer, neuropathic pain, chemotherapy induced neuropathy, acute pain, chronic pain, spasticity associated with pain, abdominal pain, abdominal pain associated with irritable bowel syndrome and/or visceral pain in a mammal.

20. A compound of formula (I) according to any one of claims 1 to 13 and 15, or a pharmaceutically acceptable salt thereof, or a pharmaceutical composition according to claim 17 for use in the treatment or prophylaxis of neuroinflammation, neurodegenerative diseases, pain, cancer, mental disorders and/or inflammatory bowel disease in a mammal.

21. A compound of formula (I) according to any one of claims 1 to 13 and 15, or a pharmaceutically acceptable salt thereof, or a pharmaceutical composition according to claim 17 for use in the treatment or prophylaxis of multiple sclerosis, Alzheimer's disease, Parkinson's disease, amyotrophic lateral sclerosis, traumatic brain injury, neurotoxicity, stroke, epilepsy, anxiety, migraine, depression, hepatocellular carcinoma, colon carcinogenesis, ovarian cancer, neuropathic pain, chemotherapy induced neuropathy, acute pain, chronic pain, spasticity associated with pain, abdominal pain, abdominal pain associated with irritable bowel syndrome and/or visceral pain in a mammal.

22. The use of a compound of formula (I) according to any one of claims 1 to 13 and 15, or a pharmaceutically acceptable salt thereof, for the preparation of a medicament for the treatment or prophylaxis of neuroinflammation, neurodegenerative diseases, pain, cancer, mental disorders and/or inflammatory bowel disease in a mammal.

23. The use of a compound of formula (I) according to any one of claims 1 to 13 and 15, or a pharmaceutically acceptable salt thereof, for the preparation of a medicament for the treatment or prophylaxis of multiple sclerosis, Alzheimer's disease, Parkinson's disease, amyotrophic lateral sclerosis, traumatic brain injury, neurotoxicity, stroke, epilepsy, anxiety, migraine, depression, hepatocellular carcinoma, colon carcinogenesis, ovarian cancer, neuropathic pain, chemotherapy induced neuropathy, acute pain, chronic pain, spasticity associated with pain, abdominal pain, abdominal pain associated with irritable bowel syndrome and/or visceral pain in a mammal.

24. A method,
wherein the method is for the treatment or prophylaxis of neuroinflammation, neurodegenerative diseases, pain, cancer, mental disorders, or inflammatory bowel disease in a mammal,
the method comprising administering an effective amount of a compound of formula (I) according to any one of claims 1 to 13 and 15, or a pharmaceutically acceptable salt thereof, or of a pharmaceutical composition according to claim 17, to the mammal.

25. A method,
wherein the method is for the treatment or prophylaxis of multiple sclerosis, Alzheimer's disease, Parkinson's disease, amyotrophic lateral sclerosis, traumatic brain injury, neurotoxicity, stroke, epilepsy, anxiety, migraine, depression, hepatocellular carcinoma, colon carcinogenesis, ovarian cancer, neuropathic pain, chemotherapy induced neuropathy, acute pain, chronic pain, spasticity associated with pain, abdominal pain, abdominal pain associated with irritable bowel syndrome, or visceral pain in a mammal,
the method comprising administering an effective amount of a compound of formula (I) according to any one of claims 1 to 13 and 15, or a pharmaceutically acceptable salt thereof, or of a pharmaceutical composition according to claim 17, to the mammal.

26. The invention as described hereinbefore.
